(19) United States
(12) Patent Application Publication
(10) Pub. No.: US 2023/0250156 A1
(43) Pub. Date: Aug. 10, 2023

(54) MULTIMERS FOR VIRAL STRAIN EVOLUTION

(71) Applicant: Quadrucept Bio Limited, London (GB)

(72) Inventors: Hanif ALI, London (GB); Jasper CLUBE, London (GB)

(21) Appl. No.: 17/909,621

(22) PCT Filed: Mar. 15, 2021
(86) PCT No.: PCT/EP2021/056576

§ 371 Date: Sep. 6, 2022

(30) Foreign Application Priority Data
Mar. 22, 2020 (GB) 2004153.9
Apr. 5, 2020 (GB) 2005008.4
May 16, 2020 (GB) 2007280.7
May 17, 2020 (GB) 2007286.4
Jun. 28, 2020 (GB) 2009847.1
Nov. 15, 2020 (GB) 2017966.9
Jan. 31, 2021 (GB) 2101331.3
Feb. 14, 2021 (GB) 2102057.3

(51) Int. Cl. C07K 16/10; G01N 33/569; A61K 9/00; A61K 39/42; A61K 45/06; A61P 31/14; C07K 14/725; A61K 38/17; C07K 14/55; A61K 38/20; C07K 16/28; A61K 39/395; C07K 16/24

(52) U.S. Cl. CPC C07K 16/10; A61K 9/0078; A61K 38/1774; A61K 38/2013; A61K 39/42; A61K 39/3955; A61K 45/06; A61P 31/14; C07K 14/55; C07K 14/7051; C07K 16/244; C07K 16/2827; C07K 16/2875; C07K 16/2878; C07K 16/2887; G01N 33/56983; A61K 2039/505; C07K 2317/569

(57) ABSTRACT

The invention relates to multimers such as multimers comprising 4 copies of a binding site or peptide; tetramers of polypeptides; and tetramers, octamers, dodecamers and hexadecamers of epitopes or effector domains, such as antigen binding sites (eg, antibody or TCR binding sites that specifically bind to antigen or pMHC, or variable domains thereof) or peptides such as incretin, insulin or hormone peptides. The invention also relates to methods and uses to expand antigen specificity of binding sites, as well as vaccines, methods of vaccination and assay methods and reagents.

### TECHNICAL FIELD

The invention relates to multimers, methods and uses to expand antigen specificity of binding sites, as well as vaccines, methods of vaccination and assay methods and reagents.

The invention also relates to multimers such as dimers or tetramers of polypeptides; and tetramers or higher-order multimers (eg, octamers, dodecamers and hexadecamers) of epitopes or effector domains, such as antigen binding sites (eg, antibody or TCR binding sites that specifically bind to antigen or pMHC, or variable domains thereof) or peptides such as incretin, insulin or hormone peptides.

### BACKGROUND

Multimers of effector domains have recognized utility in medical and non-medical applications for combining and multiplying the activity and presence of effector domains, eg, to provide for higher avidity of antigen binding (for effector domains that are antibody or TCR binding domains, for example) or for enhancing biological or binding activity, such as for providing bi- or multi-specific targeting or interaction with target ligands in vivo or in vitro.

Multimerisation domains which cause self-assembly of protein monomers into multimers are known in the art. Examples include domains found in transcription factors such as p53, p63 and p73, as well as domains found in ion channels such as TRP cation channels. The transcription factor p53 can be divided into different functional domains: an N-terminal transactivation domain, a proline-rich domain, a DNA-binding domain, a tetramerisation domain and a C-terminal regulatory region. The tetramerisation domain of human p53 extends from residues 325 to 356, and has a 4-helical bundle fold (Jeffrey et al., Science (New York, N.Y.) 1995, 267(5203): 1498-1502). The TRPM tetramerisation domain is a short anti-parallel coiled-coil tetramerisation domain of the transient receptor potential cation channel subfamily M member proteins 1-8. It is held together by extensive core packing and interstrand polar interactions (Fujiwara et al., Journal of Molecular Biology 2008, 383(4):854-870). Transient receptor potential (TRP) channels comprise a large family of tetrameric cation-selective ion channels that respond to diverse forms of sensory input. Another example is the potassium channel BTB domain. This domain can be found at the N terminus of voltage-gated potassium channel proteins, where represents a cytoplasmic tetramerisation domain (T1) involved in assembly of alpha-subunits into functional tetrameric channels (Bixby et al., Nature Structural Biology 1999, 6(1):38-43). This domain can also be found in proteins that are not potassium channels, like KCTD1 (potassium channel tetramerisation domain-containing protein 1; Ding et al., DNA and Cell Biology 2008, 27(5):257-265).

Multimeric antibody fragments have been produced using a variety of multimerisation techniques, including biotin, dHLX, ZIP and BAD domains, as well as p53 (Thie et al., Nature Boitech., 2009:26, 314-321). Biotin, which is efficient in production, is a bacterial protein which induces immune reactions in humans.

Human p53 (UniProtKB - P04637 (P53_HUMAN)) acts as a tumor suppressor in many tumor types, inducing growth arrest or apoptosis depending on the physiological circumstances and cell type. It is involved in cell cycle regulation as a trans-activator that acts to negatively regulate cell division by controlling a set of genes required for this process. Human p53 is found in increased amounts in a wide variety of transformed cells. It is frequently mutated or inactivated in about 60% of cancers. Human p53 defects are found in Barrett metaplasia a condition in which the normally stratified squamous epithelium of the lower esophagus is replaced by a metaplastic columnar epithelium. The condition develops as a complication in approximately 10% of patients with chronic gastroesophageal reflux disease and predisposes to the development of esophageal adenocarcinoma.

Nine isoforms of p53 naturally occur and are expressed in a wide range of normal tissues but in a tissue-dependent manner. Isoform 2 is expressed in most normal tissues but is not detected in brain, lung, prostate, muscle, fetal brain, spinal cord and fetal liver. Isoform 3 is expressed in most normal tissues but is not detected in lung, spleen, testis, fetal brain, spinal cord and fetal liver. Isoform 7 is expressed in most normal tissues but is not detected in prostate, uterus, skeletal muscle and breast. Isoform 8 is detected only in colon, bone marrow, testis, fetal brain and intestine. Isoform 9 is expressed in most normal tissues but is not detected in brain, heart, lung, fetal liver, salivary gland, breast or intestine.

### SUMMARY OF THE INVENTION

The invention provides; A protein multimer comprising 4 copies of a binding site, wherein the binding site is capable of binding to a virus spike protein of a coronavirus.

There is also provided:-
a pharmaceutical composition (eg, an inhalable pharmaceutical composition) comprising such a multimer;
an inhalation device (optionally a nebuliser or inhaler) comprising the composition;
a mixture of at least 2 different multimers wherein a first of said multimers comprises 4 copies of an antigen binding site that is capable of binding to a first spike antigen and a second of said multimers comprises 4 copies of an antigen binding site that is capable of binding to a second spike antigen, and the antigens are different;
a method of expanding the antigen binding specificity of a binding site, wherein the binding site binds or neutralises (eg, when administered to humans) a first antigen, but not a second antigen when the binding site is comprised in monovalent form by a protein that specifically binds to the first antigen, the method comprising providing a plurality of copies of a polypeptide, and multimerising at least 4 of the polypeptides to produce a multimer comprising at least 4 copies of the polypeptide, wherein the polypeptide comprises one, two or more copies of the binding site, whereby binding sites of the multimer are capable of binding the first and second antigens;
a multimer obtainable by said method of expanding wherein the multimer is for targeting a virus whose antigens evolve through mutation during viral infection of a human subject, optionally for treating a coronavirus infection;
a method of binding multiple copies of an antigen, the method comprising combining the copies with the multimer or composition, wherein the copies are bound by the multimer, and optionally the method comprising isolating the multimer bound to the antigen copies;
a method of detecting the presence of anti-first antigen antibodies in a bodily fluid sample of a human or animal, the method comprising carrying out an ELISA assay;
a method for detecting the presence of an antigen in a sample, the method comprising combining the sample with the multimer, allowing antigen in the sample to bind multimers to form antigen/multimer complexes and detecting antigen/multimer complexes;
a method of expanding a utility of an antigen binding site, the method comprising producing the multimer, wherein the multimer comprises at least 4 copies of the binding site;
a method for the treatment or prevention of a disease or condition in a human or animal subject), the method comprising administering to the subject a plurality of the multimers;
a multimer that is capable of binding to different forms of a virus spike protein for treating, preventing or reducing in a human or animal infection by a virus comprising a first form of spike protein, and for treating, preventing or reducing infection by a virus comprising a second form of the spike protein;
a multimer that is capable of binding to different forms of a virus spike protein for treating or preventing or reducing a seasonal viral infection in a human or animal; and
a medicament for administration to a human or animal subject for treating or preventing a seasonal virus, wherein the medicament comprises a plurality of the multimers, wherein the medicament comprises a pharmaceutically acceptable diluent, carrier or excipient.

The invention provides: A polypeptide comprising an antibody Fc region, wherein the Fc region comprises an antibody CH2 and an antibody CH3; and a self-associating multimerisation domain (SAM); wherein the CH2 comprises an antibody hinge sequence and is devoid of a core hinge region. Advantageously, the Fc does not directly pair with another Fc, which is useful for producing multimers by multimerization using SAM domains. For example, a benefit may be aiding desired multimer formation and/or enhancing multimer purity formed by such multimerization.

The invention also provides: A multimer of a plurality of antibody Fc regions, wherein each Fc is comprised by a respective polypeptide and is unpaired with another Fc region; optionally wherein the multimer is for medical use.

The invention also provides:-

### In a First Configuration

A protein multimer of at least first, second, third and fourth copies of an effector domain (eg, a protein domain or a peptide), wherein the multimer is multimerised by first, second, third and fourth self-associating tetramerisation domains (TDs) which are associated together, wherein each tetramerisation domain is comprised by a respective engineered polypeptide comprising one or more copies of said protein domain or peptide.

### In a Second Configuration

An isolated tetramer, octamer, dodecamer, hexadecamer or 20-mer of a TCR binding site, insulin peptide, incretin peptide or peptide hormone; or a plurality of said tetramers or octamers.

An isolated tetramer, octamer, dodecamer, hexadecamer or 20-mer of an antibody binding site or an antibody variable domain (eg, a single variable domain); or a plurality of said tetramers or octamers.

In an example the tetramer or octamer is soluble in aqueous solution (eg, aqueous eukaryotic cell culture medium). In an example the tetramer or octamer is expressible in a eukaryotic cell. Exemplification is provided below.

### In a Third Configuration

A tetramer, octamer, dodecamer, hexadecamer or 20-mer (eg, a tetramer or octamer) of
(a) TCR V domains or TCR binding sites, wherein the tetramer or octamer is soluble in aqueous solution (eg, an aqueous eukaryotic cell growth medium or buffer);
(b) antibody single variable domains, wherein the tetramer or octamer is soluble in aqueous solution (eg, an aqueous eukaryotic cell growth medium or buffer);
(c) TCR V domains or TCR binding sites, wherein the tetramer or octamer is capable of being intracellularly and/or extracellularly expressed by HEK293 cells; or
(d) antibody variable domains (eg, antibody single variable domains), wherein the tetramer or octamer is capable of being intracellularly and/or extracellularly expressed by HEK293 cells.

### In a Fourth Configuration

An engineered polypeitide or monomer of a multimer, tetramer, octamer, dodecamer, hexadecamer or 20-mer (eg, a tetramer or octamer) of the invention.

### In a Fifth Configuration

An engineered (and optionally isolated) engineered polypeptide (P1) which comprises (in N- to C-terminal direction):-
(a) TCR V1-TCR C1 - antibody CH1 (eg, IgG CH1) - optional linker - TD, wherein
   (i) V1 is a Vα and C1 is a Cα;
   (ii) V1 is a Vβ and C1 is a Cβ;
   (iii) V1 is a Vγ and C1 is a Cy; or
   (iv) V1 is a Vδ and C1 is a Cδ;
   or
(b) TCR V1 - antibody CH1 (eg, IgG CH1) - optional linker - TD, wherein
   (i) V1 is a Vα;
   (ii) V1 is a Vβ;
   (iii) V1 is a Vy; or
   (iv) V1 is a Vδ;
   or
(c) antibody V1 - antibody CH1 (eg, IgG CH1) - optional linker - TD, wherein
   (i) V1 is a VH; or
   (ii) V1 is a VL (eg, a Vλ, or a Vκ);
   or
(d) antibody V1 - optional antibody CH1 (eg, IgG CH1) - antibody Fc (eg, an IgG Fc) - optional linker - TD, wherein
   (i) V1 is a VH; or
   (ii) V1 is a VL (eg, a Vλ or a Vκ);
   or
(e) antibody V1 - antibody CL (eg, a Cλ or a Cκ) - optional linker - TD, wherein
   (i) V1 is a VH; or
   (ii) V1 is a VL (eg, a Vλ or a Vκ);
   or
(f) TCR V1 -TCR C1 - optional linker - TD, wherein
   (i) V1 is a Vα and C1 is a Cα;
   (ii) V1 is a Vβ and C1 is a Cβ;
   (iii) V1 is a Vγ and C1 is a Cy; or
   (iv) V1 is a Vδ and C1 is a Cδ.

### In a Sixth Configuration

A nucleic acid encoding an engineered polypeptide or monomer of the invention, optionally wherein the nucleic acid is comprised by an expression vector for expressing the polypeptide.

### In a Seventh Configuration

Use of a nucleic acid or vector of the invention in a method of manufacture of protein multimers for producing intracellularly expressed and/or secreted multimers, wherein the method comprises expressing the multimers in and/or secreting the multimers from eukaryotic cells comprising the nucleic acid or vector.

### In an Eighth Configuration

A method producing
(a) TCR V domain multimers, the method comprising the soluble and/or intracellular expression of TCR V-TD (eg, NHR2 TD or TCR V- p53 TD) fusion proteins expressed in eukaryotic cells, the method optionally comprising isolating a plurality of said multimers;
(b) antibody V domain multimers, the method comprising the soluble and/or intracellular expression of antibody V (eg, a single variable domain)-TD (eg, V-NHR2 TD or V- p53 TD) fusion proteins expressed in eukaryotic cells, the method optionally comprising isolating a plurality of said multimers;
(c) incretin peptide (eg, GLP-1, GIP or insulin) multimers, the method comprising the soluble and/or intracellular expression of incretin peptide-TD (eg, incretin peptide-NHR2 TD or incretin peptide-p53 TD) fusion proteins expressed in eukaryotic cells, such as HEK293T cells; the method optionally comprising isolating a plurality of said multimers; or
(d) peptide hormone multimers, the method comprising the soluble and/or intracellular expression of peptide hormone-TD (eg, peptide hormone-NHR2 TD or peptide hormone- p53 TD) fusion proteins expressed in eukaryotic cells, such as HEK293T cells; the method optionally comprising isolating a plurality of said multimers.

### In a Ninth Configuration

Use of a nucleic acid or vector of the invention in a method of manufacture of protein multimers for producing glycosylated multimers in eukaryotic cells comprising the nucleic acid or vector.

### In a Tenth Configuration

Use of self-associating tetramerisation domains (TD) (eg, NHR2 TD, p53 TD, p63 TD or p73 TD or a homologue or orthologue thereof) in a method of the manufacture of a tetramer of polypeptides, for producing a higher yield of tetramers versus monomer and/or dimer polypeptides.

### In an Eleventh Configuration

Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).

### In a Twelfth Configuration

Use of self-associating tetramerisation domains (TD) (eg, NHR2 TD, p53 TD, p63 TD or p73 TD or a homologue or orthologue thereof) in a method of the manufacture of a tetramer of a polypeptide, for producing a plurality of tetramers that are not in mixture with monomers, dimers or trimers.

### In a Thirteenth Configuration

A eukaryotic host cell comprising the nucleic acid or vector for intracellular and/or secreted expression of the multimer, tetramer, octamer, dodecamer, hexadecamer or 20-mer (eg, tetramer, octamer), engineered polypeptide or monomer of the invention.

### In a Fourteenth Configuration

Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).

### In a Fifteenth Configuration

A multivalent heterodimeric soluble T cell receptor capable of binding pMHC complex comprising:
(i) TCR extracellular domains;
(ii) immunoglobulin constant domains; and
(iii) an NHR2 multimerisation domain of ETO.

### In a Sixteenth Configuration

A multimeric immunoglobulin, comprising
(i) immunoglobulin variable domains; and
(ii) an NHR2 multimerisation domain of ETO.

### In a Seventeenth Configuration

method for assembling a soluble, multimeric polypeptide, comprising:
(a) providing a monomer of the said multimeric polypeptide, fused to an NHR2 domain of ETO;
(b) causing multiple copies of said monomer to associate, thereby obtaining a multimeric, soluble polypeptide.

### In an Eighteenth Configuration

A mixture comprising (i) a cell line (eg, a eukaryotic, mammalian cell line, eg, a HEK293, CHO or Cos cell line) encoding a polypeptide of the invention; and (ii) tetramers of the invention.

### In a Ninteenth Configuration

A method for enhancing the yield of tetramers of an protein effector domain (eg, an antibody variable domain or binding site), the method comprising expressing from a cell line (eg, a mammalian cell, CHO, HEK293 or Cos cell line) tetramers of a polypeptide, wherein the polypeptide is a polypeptide of the invention and comprises one or more effector domains; and optionally isolating said expressed tetramers.

### In a Twentieth Configuration

A polypeptide comprising (in N- to C-terminal direction; or in C- to N-terminal direction)
(i) An immunoglobulin superfamily domain;
(ii) An optional linker; and
(iii) A self-associating multimerisation domain (SAM) (optionally a self-associating tetramerisation domain (TD)).

### In a Twenty-First Configuration

A method of expanding the antigen binding specificity of a binding site, wherein the binding site binds a first antigen, but not a second antigen (eg, when administered to humans) when the binding site is comprised in monovalent or bivalent form by a protein that specifically binds to the first antigen, the method comprising providing a plurality of copies of a polypeptide of the invention, and multimerising at least 4 of the polypeptides to produce a multimer comprising at least 4 copies of the polypeptide, wherein the polypeptide comprises one, two or more copies of the binding site, whereby binding sites of the multimer are capable of binding the first and second antigens.

### In a Twenty-Second Configuration

Use of a polyepeptide of the invention in a method of manufacturing a multimer for expanding the antigen binding specificity of a binding site, wherein the binding site binds a first antigen, but not a second antigen (eg, when administered to humans) when the binding site is comprised in monovalent or bivalent form by a protein that specifically binds to the first antigen, wherein the method comprises providing a plurality of copies of a polypeptide of the invention, and multimerising at least 4 of the polypeptides to produce a multimer comprising at least 4 copies of the polypeptide, wherein the polypeptide comprises one, two or more copies of the binding site, whereby binding sites of the multimer are capable of binding the first and second antigens.

In an embodiment, the polypeptide comprises aspects useful for treating or preventing a viral infection or cancer wherein the polypeptide comprises
A: one or more epitope binding sites, optionally wherein the binding site binds to (i) a SARS-Cov-2 antigen (eg, a SARS-Cov-1 antigen and a SARS-Cov-2 antigen); (ii) BCMA (B-cell maturation antigen) and TACI (transmembrane activator and calcium modulator and cyclophilin ligand interactor); (iii) first and second Coronovirus antigens; (iv) first and second HIV antigens; (v) first and second P falciparum antigens; (vi) first and second Salmonella antigens; (vii) a TMPRSS protein (eg, a TMPRSS2 antigen); or (viii) a ACE2 antigen; or
B: one, two or more copies of an ACE2 peptide (eg, an ACE2 extracellular domain) and/or a TMPRSS2 peptide (eg, a TMPRSS protein extracellular domain); or
C: a first binding site that binds to a Coronavirus antigen (eg, a SARS-Covantigen, preferably a SARS-Cov-1 or -2 antigen) and one or more copies of an ACE2 peptide (eg, an ACE2 extracellular domain) and/or a TMPRSS2 peptide (eg, a TMPRSS protein extracellular domain); or
D: a first binding site that binds to a ACE2 extracellular domain; and one or more copies of an ACE2 peptide (eg, an ACE2 extracellular domain) and/or a TMPRSS2 peptide (eg, a TMPRSS protein extracellular domain); or
E: a first binding site that binds to a TMPRSS2 peptide extracellular domain; and one or more copies of an ACE2 peptide (eg, an ACE2 extracellular domain) and/or a TMPRSS2 peptide (eg, a TMPRSS protein extracellular domain).

The invention also provides:
A protein multimer comprising more than 2 copies of a binding site, wherein the binding site is capable of binding to a first antigen, optionally wherein the multimer is capable of binding to the first antigen and a second antigen, wherein the antigens are different.
For example, the multimer comprises from 4 to 32 (eg, from 4 to 24, or from 4 to 20, or from 4 to 16) copies of the binding site, ie, this means that the multimer does not comprise any more or less than said number. In an embodiment, the multimer comprises, 4, 5, 6, 7, 8, 9, 10, 11, 12, 13, 14, 15, 16, 17, 18, 19, 20, 21, 22, 23, 24, 25, 26, 27, 28, 29, 30, 31 or 32 copies of the binding site.
For example, the multimer contains from 4 to 32 (eg, from 4 to 24, or from 4 to 20, or from 4 to 16) copies of the binding site, ie, this means that the multimer does not contain any more or less than said number. In an embodiment, the multimer contains, 4, 5, 6, 7, 8, 9, 10, 11, 12, 13, 14, 15, 16, 17, 18, 19, 20, 21, 22, 23, 24, 25, 26, 27, 28, 29, 30, 31 or 32 copies of the binding site.
In an embodiment, a control protein multimer comprising 1 or 2 (but no more than 1 or 2 respectively) of said binding sites is not capable of binding to the first antigen; or is capable of binding to the first antigen, but not to the second antigen. Binding may be determined by an ELISA assay, such as by determining OD₄₅₀, for example in an ELISA assay described herein.
An aspect provides: A protein multimer comprising more than 2 copies of a binding site, wherein the binding site is capable of binding to a virus spike protein of a first virus, optionally wherein the multimer is capable of binding to the first and a second virus, wherein the viruses are different. This is exemplified herein for 2 different viruses.
An aspect provides:-A method for detecting the presence of an antigen in a sample, the method comprising combining the sample with a multimer of the invention, allowing antigen in the sample to bind multimers to form antigen/multimer complexes and detecting antigen/multimer complexes.
A method of expanding a utility of an antigen (eg, a protein) binding site, the method comprising producing a multimer of the invention, wherein the multimer comprises a plurality of copies (eg, at least 4 or 8 copies) of the binding site.

The invention also provides a pharmaceutical composition, cosmetic, foodstuff, beverage, cleaning product, detergent comprising the multimer(s), tetramer, octamer, dodecamer, hexadecamer or 20-mer (eg, tetramer(s) or octamer(s)) of the invention.

A multimer herein is, eg, a dimer, trimer, tetramer, octamer, dodecamer, hexadecamer or 20-mer.

As demonstrated in Example 22, dodecamer and hexadecamer multimers surprisingly display a very high functional affinity for antigen binding due to the increasing avidity effect. The functional affinity for these going from 8 to 12 binding sites (compare Tables 15 and 16) or from 8 to 16 binding sites is much more than additive; a synergistic increase is seen as a result of enhanced avidity. Thus, in one embodiment, a multimer which is 12-valent for an antigen (ie, a dodecamer as described herein) is preferred; in another embodiment a multimer which is 16-valent for an antigen (ie, hexadecamer as described herein) is preferred.

Thus, a further configuration of the invention provides:
A protein multimer comprising or containing 4 copies of a peptide or an antigen binding site, (optionally wherein the antigen is a virus spike protein of a first virus, optionally wherein the multimer is capable of binding to the first and a second virus, wherein the viruses are different).
A protein multimer comprising or containing 8 copies of a peptide or an antigen binding site, (optionally wherein the antigen is a virus spike protein of a first virus, optionally wherein the multimer is capable of binding to the first and a second virus, wherein the viruses are different).
A protein multimer comprising or containing 12 copies of a peptide or an antigen binding site, (optionally wherein the antigen is a virus spike protein of a first virus, optionally wherein the multimer is capable of binding to the first and a second virus, wherein the viruses are different).
A protein multimer comprising or containing 16 copies of a peptide or an antigen binding site, (optionally wherein the antigen is a virus spike protein of a first virus, optionally wherein the multimer is capable of binding to the first and a second virus, wherein the viruses are different).
A protein multimer comprising or containing 20 copies of a peptide or an antigen binding site, (optionally wherein the antigen is a virus spike protein of a first virus, optionally wherein the multimer is capable of binding to the first and a second virus, wherein the viruses are different).
A protein multimer comprising or containing 24 copies of a peptide or an antigen binding site, (optionally wherein the antigen is a virus spike protein of a first virus, optionally wherein the multimer is capable of binding to the first and a second virus, wherein the viruses are different).

### In a Twenty-Third Configuration

As demonstrated in Example 32, multimers comprising 4 copies of an antigen binding site of REGN10987, REGN10933 or CB6 surprisingly display much improved neutralization potency of the Quad formats over the parental IgG format (FIG. 61-J), with a a 600-fold improvement in neutralization potency over the parental mAb being surprisingly achieved. Thus, the present configuration provides:
A protein multimer comprising 4 copies of an antigen binding site of an antibody, wherein the antibody is selected from REGN10987, REGN10933 and CB6.
A protein multimer comprising 4 copies of an antigen binding site of an antibody, wherein the multimer comprises a dimer of an antibody or a fragment thereof (eg, a Fab), wherein the antibody is selected from REGN10987, REGN10933 and CB6.
A protein multimer comprising a dimer of an antibody or a fragment thereof (eg, a Fab), wherein the antibody is selected from REGN10987, REGN10933 and CB6.
Advantageously, the multimer may comprise mammalian cell glycosylation. The multimer may, for example, comprise 8, 12, 16, 20 or 24 copies of said binding site in certain aspects of the configuration.

### In a Twenty-Fourth Configuration

As demonstrated in Example 33, multimers comprising 4 copies of antigen binding domain Nb-112 (a VHH domain comprising the amino acid sequence of SEQ ID NO: 288) surprisingly display much improved neutralization potency of the Quad formats over the parental VHH format (FIGS. 62D & 62E), with a substantial improvement in neutralization potency over the parental VHH being surprisingly achieved. As demonstrated in Example 33, such multimers also surprisingly show substantially higher binding to protein A, which aids purification. Thus, the present configuration provides:
A protein multimer comprising 4 (and optionally no more than 4) copies of Nb-112 (SEQ ID NO: 288).
A protein multimer comprising 4 (and optionally no more than 4) copies of an antibody variable domain comprising the amino acid sequence SEQ ID NO: 288.
A protein multimer comprising 8 (and optionally no more than 8) copies of Nb-112 (SEQ ID NO: 288).
A protein multimer comprising 8 (and optionally no more than 8) copies of an antibody variable domain comprising the amino acid sequence SEQ ID NO: 288.
A protein multimer comprising 12 (and optionally no more than 12) copies of Nb-112 (SEQ ID NO: 288).
A protein multimer comprising 12 (and optionally no more than 12) copies of an antibody variable domain comprising the amino acid sequence SEQ ID NO: 288.
A protein multimer comprising 16 (and optionally no more than 16) copies of Nb-112 (SEQ ID NO: 288).
A protein multimer comprising 16 (and optionally no more than 16) copies of an antibody variable domain comprising the amino acid sequence SEQ ID NO: 288.
A protein multimer comprising 20 (and optionally no more than 20) copies of Nb-112 (SEQ ID NO: 288).
A protein multimer comprising 20 (and optionally no more than 20) copies of an antibody variable domain comprising the amino acid sequence SEQ ID NO: 288.
A protein multimer comprising 24 (and optionally no more than 24) copies of Nb-112 (SEQ ID NO: 288).
A protein multimer comprising 24 (and optionally no more than 24) copies of an antibody variable domain comprising the amino acid sequence SEQ ID NO: 288.
A protein multimer comprising 28 (and optionally no more than 28) copies of Nb-112 (SEQ ID NO: 288).
A protein multimer comprising 28 (and optionally no more than 28) copies of an antibody variable domain comprising the amino acid sequence SEQ ID NO: 288.
Advantageously, the multimer may comprise mammalian cell glycosylation.

### In a Twenty-Fifth Configuration

A method of purifying a multimer of the invention from a composition comprising the multimer, the method comprising contacting the composition with an antigen (eg, a supergantigen) and binding the multimer to the antigen, and optionally isolating antigen/multimer complexes.

The multimer may be obtained from the complexes.

Preferably, the multimer comprises at least 4 copies (eg, 4, 8, 12, 16, 20, 24 or 28 copies) of a VH3 family domain and the superantigen is Protein A. Preferably, the multimer comprises at least 4 copies (eg, 4, 8, 12, 16, 20, 24 or 28 copies) of a Vκ domain and the superantigen is Protein L.

### In a Twenty-Sixth Configuration

The polypeptide described herein may, for example, comprise binding domain QB-GB or a binding domain (eg, an antibody single variable domain) that competes with QB-GB for binding to SARS-CoV-2 spike in an in vitro competition assay.

The polypeptide described herein may, for example, comprise binding domain QB-GB or a binding domain (eg, an antibody single variable domain) that binds to the same SARS-CoV-2 spike epitope (or an overlapping epitope) as QB-GB.

The polypeptide described herein may, for example, comprise a binding domain that binds to the inner face of the RBD (receptor-binding domain) of SARS-CoV-2 spike.

The polypeptide described herein may, for example, comprise a binding domain that binds to the inner face of the RBD (receptor-binding domain) of SARS-CoV-2 spike that is in the up state.

Similarly, the multimer herein may comprise copies of such a binding domain. The multimer described herein may, for example, bind to the inner face of the RBD (receptor-binding domain) of SARS-CoV-2 spike. The multimer described herein may, for example, bind to the inner face of the RBD (receptor-binding domain) of SARS-CoV-2 spike that is in the up state.

As explained in Example 37 Quad multimers that have such features have been found to be highly advantageous and may be more resistant to receptor-driven selection pressure associated with SARS-Cov-2 mutation.

The invention also provides polypeptide dimers, as well as tetramers of dimers.

### BRIEF DESCRIPTION OF THE DRAWINGS

FIG. 1: A schematic drawing representing the stepwise self-assembly of a tetravalent heterodimeric soluble TCR protein complex via a monomer and homodimer, which is aided by NHR2 tetramerisation domain.

FIG. 2: A schematic drawing representing the stepwise self-assembly of an octavalent heterodimeric soluble TCR protein complex via a monomer² and homodimer², which is aided by NHR2 tetramerisation domain and immunoglobulin hinge domain.

FIG. 3: A schematic drawing of the domain arrangements in the α and β chain used for expressing and assembling ts-NY-ESO-1 TCR.

FIG. 4: A schematic drawing of the domain arrangements in the α and β chain used for expressing and assembling os-NY-ESO-1 TCR.

FIG. 5: Amino acid sequence of the α and β chain of the ts-NY-ESO-1 TCR protein complex. Amino acid sequences of alternate domains are underlined.

FIG. 6: Amino acid sequence of the α and β chain of the os-NY-ESO-1 TCR protein complex. Amino acid sequences of alternate domains are underlined.

FIG. 7: A schematic drawing of the domain arrangements in the α and β chain used for expressing and assembling ts-NY-ESO-1 TCR-IL2 fusion protein complex.

FIG. 8: A schematic drawing of the domain arrangements in the α and β chain used for expressing and assembling os-NY-ESO-1 TCR-IL2 fusion protein complex.

FIG. 9: Amino acid sequence of the α and β chain of the ts-NY-ESO-1 TCR-IL2 fusion protein complex. Amino acid sequences of alternate domains are underlined.

FIG. 10: Amino acid sequence of the α and β chain of the os-NY-ESO-1 TCR-IL2 fusion protein complex. Amino acid sequences of alternate domains are underlined.

FIG. 11A: A schematic drawing representing the stepwise self-assembly of a tetravalent single domain antibody (dAb) complex via a monomer and homodimer, which is aided by NHR2 tetramerisation domain.

FIG. 11B: A schematic drawing of the domain arrangements for assembly of tetravalent dAbs, including linker and NHR2 domains.

FIG. 12A: A schematic drawing representing the stepwise self-assembly of a tetravalent Fab complex via a monomer and homodimer, which is aided by NHR2 tetramerisation domain.

FIG. 12B: A schematic drawing of the domain arrangements for assembly of tetravalent Fabs, including linker and NHR2 domains in the heavy chain, and light chain variable and constant domains.

FIG. 13A: A schematic drawing representing the stepwise self-assembly of an octavalent Fab complex via a monomer and homodimer, which is aided by NHR2 tetramerisation domain and an antibody hinge region linked to CH1 domain.

FIG. 13B: A schematic drawing of the domain arrangements for assembly of octavalent Fabs, including hinge, linker and NHR2 domains in the heavy chain, and light chain variable and constant domains.

FIG. 14: is a schematic of Quad 16 and Quad 17.

FIG. 15: shows (A) Quad 16 and (B) Quad 17 monomer sequences and configuration.

FIG. 16: shows analysis of secreted proteins using anti-Ig Western Blot: (A) a PAGE gel under SDS denatured conditions - 16=Quad16; 17=Quad17; and (B) a PAGE gel under native (ie, non-denatured) conditions - 16=Quad16; 17=Quad17.

FIG. 17: Western blots prepared from denaturing SDS-PAGE gel probed with anti-human IgG HRP detection antibody (A) Protein samples from Quads 3 and 4 were prepared from whole cell extracts and loaded in lanes 1 and 2 respectively. The expected Mw for Quads 3 and 4 are 46.1 and 46.4 kDa respectively. (B) Protein samples from Quads 12 and 13 were prepared from whole cell extracts and loaded in lanes 1 and 2 respectively. The expected Mw for Quads 12 and 13 are 47.8 and 48.1 kDa respectively.

FIG. 18: Western blots prepared from denaturing SDS-PAGE gel probed with anti-human IgG HRP detection antibody (A) Protein samples from Quads 3 and 4 were prepared by concentrating cell supernatant and loaded in lanes 1 and 2 respectively. The expected Mw for Quads 3 and 4 are 46.1 and 46.4 kDa respectively. (B) Protein samples from Quads 12 and 13 were prepared by concentrating cell supernatant and loaded in lanes 1 and 2 respectively. The expected Mw for Quads 12 and 13 are 47.8 and 48.1 kDa respectively.

FIG. 19: Western blots prepared from denaturing SDS-PAGE gel probed with anti-HIS HRP detection antibody (A) Protein samples from Quads 14, 15, 18 and 19 were prepared from whole cell extracts and loaded in lanes 1-4, respectively. The expected Mw for Quads 14, 15, 18 and 19 are 22.0, 22.3, 37.4 and 37.7 kDa respectively. (B) Protein samples from Quads 23, 24, 26 and 27 were prepared from whole cell extracts and loaded in lanes 1-4, respectively. The expected Mw for Quads 23, 24, 26 and 27 are 32.1, 32.4, 33.7 and 34.0 kDa respectively. (C) Protein samples from Quads 34, and 38 were prepared from whole cell extracts and loaded in lanes 1-2, respectively. The expected Mw for Quads 34, and 38 are 25.5 and 25.4 kDa respectively. (D) Protein samples from Quads 40, 42, 44 and 46 were prepared from whole cell extracts and loaded in lanes 1-4, respectively. The expected Mw for Quads 40, 42, 44 and 46 are 25.4, 37.6, 25.5 and 38.0 kDa respectively. Lane U contains concentrated serum prepared from untransfected HEK293T cells (negative control) and C is a His-tagged protein used as a positive control for the anti-His HRP detection antibody. Serum anti-His background band is highlighted by a black arrow, which can be consistently detected in all for blots.

FIG. 20: Western blot prepared from denaturing SDS-PAGE gel (A) and probed with anti-human IgG HRP detection antibody. Protein samples from Quads 14 and 15 were prepared from whole cell extracts and loaded in lanes 1 and 2, respectively. The expected Mw for Quads 14 and 15 are 22.0 and 22.3 kDa respectively. (B) Western blot prepared from Native PAGE gels and probed with anti-human IgG HRP detection antibody. Lanes 1 and 2 contains protein samples from Quads 14 and 15 prepared from whole cell extract.

FIG. 21: Quad polyeptides fused to leader and tag sequences. Where linker is present, the linker is G4S (only 1 G4S). * denotes TCR constant domains with introduced cysteine residue allowing S-S bond formation between TCR alpha and beta chain. Human IgG1 hinge was used. All C regions are human. The TCR V domains are specific for NY-ESO-1. GFP=green fluorescent protein.

FIG. 22: Schematic representations of the multimeric structure of Quad formats A - AC. The description and the monomeric building block from which the tetravalent Quad molecules are assembled from are described in Table 8.

FIG. 23: SDS-PAGE analysis of monospecific tetravalent dAb Quad 57 protein purified from culture supernatant. Quad protein migrated according to its expected MW as indicated by the arrow with no visible impurities.

FIG. 24: SDS-PAGE analysis of bispecific tetravalent dAb Quad 54 protein purified from culture supernatant (A). Quad protein migrated according to its expected MW as indicated by the arrow with no visible impurities (B) Direct binding ELISA using serially diluted Quad 54 protein with a fixed concentration of recombinant TNFa protein coated on plate. Quad 54 binds TNFa protein in a dose-dependent manner.

FIG. 25: SDS-PAGE analysis of monospecific tetravalent scFv Quads. Quads 51 and 63 proteins purified from culture supernatant and analysed by SDS-PAGE (A). Quad proteins migrated according to their expected MW as indicated by the arrows with no visible impurities (B) Direct binding ELISA using serially diluted Quad 51 and 63 proteins with a fixed concentration of recombinant TNFa protein coated on plate. Both Quads 51 and 63 bind TNFa protein with similar binding strength in a dose-dependent manner. (C) SDS-PAGE analysis of W51ScFv monovalent anti-TNFa control protein. (D) Western blot analysis of TNFa-mediated Caspase-3 signaling in the presence of Quad 51, Humira (Hum) and W51 ScFv. Culture medium (CM) alone or with actinomycin D (AD) were used as a negative control. The detection antibody used for each blot is indicated next to each blots. The Western blots detected by anti-Tubulin represents internal loading control. (E) SDS-PAGE analysis of Quad 53 Tet protein purified from culture supernatant. The Quad protein migrated according to its expected MW as indicated by the arrow. (F). Direct binding ELISA using serially diluted Quad 53 Tet protein with a fixed concentration of recombinant CD20 protein coated on plate. Quad 53 Tet binds CD20 protein in a dose-dependent manner.

FIG. 26: SDS-PAGE analysis of monospecific octavalent scFv Quad 53 protein purified from culture supernatant (A). Quad protein migrated according to its expected MW as indicated by the arrow with no visible impurities (B) Direct binding ELISA using serially diluted Quad 53 Oct protein with a fixed concentration of recombinant CD20 protein coated on plate. Quad 53 Oct binds CD20 protein in a dose-dependent manner. (C) Monovalent, tetravalent and octavalent version of Quad 53 analysed by SDS-PAGE. (D) Direct binding ELISA comparing binding strength of monovalent, tetravalent and octavalent version of Quad 53 to recombinant CD20. An increasing in binding strength can be seen with increasing valency of Quad 53.

FIG. 27: SDS-PAGE analysis of bispecific tetravalent scFv Quad 55 protein purified from culture supernatant (A). Quad protein migrated according to its expected MW as indicated by the arrow with no visible impurities (B) Direct binding ELISA using serially diluted Quad 55 protein with a fixed concentration of recombinant TNFa protein coated on plate. Quad 55 binds TNFa protein in a dose-dependent manner.

FIG. 28: SDS-PAGE analysis of bispecific tetravalent Quads. Bispecific scFv x dAb Quad 56 protein purified from culture supernatant and analysed by SDS-PAGE (A). Quad protein migrated according to its expected MW as indicated by the arrow with no visible impurities (B) Direct binding ELISA using serially diluted Quad 56 protein with a fixed concentration of recombinant TNFa protein coated on plate. Quad 56 binds TNFa protein in a dose-dependent manner. (C) Direct binding ELISA comparing binding strength of three different bispecific tetravalent Quad formats (Quad 54: dAb x dAb, Quad 55: scFv x scFv and Quad 56: ScFv x dAb) to recombinant CD20. All three bispecific tetravalent Quad formats bind CD20 with similar binding strength and in a dose-dependent manner.

FIG. 29: SDS-PAGE analysis of monospecific tetravalent monomeric Ig scFv Quad 64 version 1 protein purified from culture supernatant (A). “Mononomeric Ig” refers to a multimer of a polypeptide of the invention that comprises an Fc, wherein CH2 comprises a hinge sequence but lacks a core hinge region; this advantageously prevents Fc regions from multimerizing together so that the multimerization is instead brought about by the SAM (eg, TD) domains of polypeptides in the multimer. Quad protein migrated according to its expected MW as indicated by the arrow with no visible impurities (B) Direct binding ELISA using serially diluted Quad 64 protein with a fixed concentration of recombinant CD20 protein coated on plate. Quad 64 binds CD20 protein in a dose-dependent manner.

FIG. 30: SDS-PAGE analysis of monospecific tetravalent monomeric Ig scFv Quad 65 version 2 protein purified from culture supernatant (A). Quad protein migrated according to its expected MW as indicated by the arrow with no visible impurities (B) Direct binding ELISA using serially diluted Quad 65 protein with a fixed concentration of recombinant CD20 protein coated on plate. Quad 65 binds CD20 protein in a dose-dependent manner.

FIG. 31: Schematic representation of Quad 68 and Quad 69 (A & B). The specificity of dAbs for PD-L1 and 4-1BB is indicated by arrows. (C & D) SDS-PAGE analysis of Quad 68 and Quad 69 protein purified from culture supernatant. Quad proteins migrated according to their expected MW as indicated by the arrows with no visible impurities.

FIG. 32: Alignment of p53 tetramerisation domain (TD) from different species. Sequence variations from human TD are highlighted in bold and underlined.

FIG. 33. Schematic representation of the molecular (A) and structural (B & C) arrangements of tetravalent and octavalent anti-TNF alpha dAb monomeric Ig Quads. Purified Quad proteins were analysed by SDS-PAGE (D). The tetravalent and octavalent Quad proteins migrated according to the expected molecular weight as indicated with no visible impurities. The core hinge region was removed in these formats and this is indicated in the figures with either a * or as CH2′. The Q92 chain contains a His-tag located at the C-terminus, which is not shown in the figure.

FIG. 34. SDS-PAGE analysis of octavalent bispecific anti-PDL1/4-1BB dAb monomeric Ig Quad (A) and octavalent monospecific anti-PDL1 dAb monomeric Ig Quad (B) proteins purified from culture supernatant. Quad proteins migrated according to their expected molecular weight as indicated by the arrow with no visible impurities.

FIG. 35. SDS-PAGE analysis of avelumab Fab monomeric Ig Quad (A) and Humira Fab monomeric Ig Quad (B) proteins purified from culture supernatant. Quad proteins migrated according to their expected molecular weight as indicated by the arrow with no visible impurities. Humira (adalimumab) Fab monomeric Ig Quad was further analysed by SEC where the fully assembled tetrameric protein eluted as a single peak at the expected molecular weight (315.8 kDa) with no visible detection of the dimeric or monomeric form.

FIG. 36. Schematic representation of the structural arrangements of aflibercept monomeric Ig Quad (Q96). The * denotes the hinge region is divoid of the core hinge region. Q96 Quad protein was analysed by SDS-PAGE where a single protein band at the expected molecular weight can be seen (B). Binding profile of Q96 to VEGF-A was analysed by ELISA binding assay where a dose-dependent binding can be seen.

FIG. 37. Schematic representation of the molecular arrangements of monovalent, tetravalent and octavalent anti-TNF alpha dAb Quads (A). All constructs contain a His-tag located at the C-terminus, which is not shown in the figure. Purified Quad proteins were analysed by SDS-PAGE where a single protein band at the expected molecular weight for monovalent, tetravalent and octavalent versions (Lanes 1-3 respectively) can be seen (B). TNF alpha binding assay using Q88 monovalent, tetravalent and octavalent Quad proteins (C) in addition to Q92 and Q92+Q93 (D) Quad proteins were performed by ELISA where a dose-dependent binding can be seen. The respective TNF alpha neutralization potency of the Quad proteins were analysed using WEHI cell-based bioassay (E & F, respectively). Enhancement in TNF alpha neutralization potency can be seen in Quads with increasing number of anti-TNF alpha dAb binding domains.

FIG. 38. Schematic representation of the molecular (A) and structural (B) arrangements of dodeca and hexadeca anti-TNF alpha dAb multimers (we alternatively call multimers, Quads). The light chain constant region is denoted by CL, which could comprise of either kappa constant region or lambda constant region. The Q142 construct contains a His-tag located at the C-terminus, which is not shown in the figure. Purified Quad proteins were analysed by SDS-PAGE (C). Dodeca valent anti-TNF alpha dAb Quads with either lambda (lane 1) or kappa (lane 2) constant region migrated on the SDS-PAGE gel according to the expected molecular weight. Hexadeca valent anti-TNF alpha dAb Quads with either lambda (lane 3) or kappa (lane 4) constant region also migrated on the SDS-PAGE gel according to the expected molecular weight. The TNF alpha neutralization potency of the dodeca and hexadeca anti-TNF alpha dAb Quad proteins with either kappa (D) or lambda (E) light chain constant region where analysed using WEHI cell-based bioassay. Potency enhancement with increasing anti-TNF alpha dAb binding domains can be seen.

FIG. 39. Schematic structural representation of dodeca-valent trispecific (A) and hexadeca-valent tetraspecific (B) multimers. For each format, the schematic structure of the monomeric building block is also shown. The regions within these molecules containing optional flexible linkers are indicated with arrows. The CL domain could be either a kappa or lambda C region. The dodeca-valent trispecific Quad contains three different dAbs labeled 1-3, which can bind either three different antigens on three different cells or bind three antigens on the same cell or three different epitopes on the same antigen. This format represents a 4 + 4 + 4 trispecific dodeca-valent Quad. The hexadeca-valent tetraspecific Quad contains four different dAbs labeled 1-4, which can bind either four different antigens on four different cells or bind four antigens on the same cell or four epitopes on the same antigen. This format represents a 4 + 4 + 4 + 4 tetraspecific hexadeca-valent Quad.

FIG. 40. Schematic structural representation of tetravalent non Ig-like Humira Fab-TD Quad (A). The schematic structure of the monomeric building block is also shown. The regions within this molecule containing optional flexible linkers are indicated with arrows (eg, each linker is a (G₄S linker as described herein). The * denotes absence of the core hinge region (ie, the presence of a lower hinge sequence and optionally also an upper hinge sequence). Purified Humira Fab-TD protein was analysed by SDS-PAGE where a single protein band at the expected molecular weight can be seen (B). TNFα binding assay using Humira Fab-TD and Humira Fab monovalent control proteins were performed by ELISA where a dose-dependent binding can be seen (C). The respective TNFα neutralization potency of the Quad proteins were analysed using WEHI cell-based bioassay (D). Enhancement in TNFα neutralization potency can be seen in Humira Fab-TD compared to Humira Fab monovalent control.

FIG. 41. Schematic structural representation of octavalent Fabs as non Ig-like Quad A) or as Ig-like Quad (B). For each format, the schematic structure of the monomeric building block is also shown. The regions within these molecules containing optional flexible linkers are indicated with arrows (eg, each linker is a (G₄S linker as described herein).

FIG. 42. Schematics of Quad formats based on binding sites for anti-Target X (a cell-surface receptor) and gel showing expression of Quad constructs.

FIG. 43. Multimers of the invention surprisingly expand antigen specificity for predetermined binding site: Results of binding assays for Target X and a second cell-surface receptor (Target Y); tetravalent Quad constructs comprise 4 anti-X binding dAb sites and these constructs surprisingly also could bind to Y; constructs comprising one or two anti-X binding sites could only by target-X.

FIG. 44. Multimers of the invention surprisingly expand antigen specificity for predetermined binding site:Results of binding assays for Target X and a second cell-surface receptor (Target Y); octavalent Quad constructs comprise 8 anti-X binding dAb sites and these constructs surprisingly also could bind to Y; a construct comprising one anti-X binding site could only by target-X.

FIG. 45. Schematic representation of SARS-CoV-2 diagnostic and serological tests set-up using multivalent reagents. Schematic representation of the multimeric reagents shown in FIGS. 45A-E utilizing p53-TD and non-multimeric reagents used to develop SARS-CoV-2 diagnostic and serological tests (FIGS. 45F-G). Detection of viral antigens using a pair of non-competing standard IgG antibodies. The first antibody is used to coat a solid surface and capture the viral antigen. The second antibody is used to specifically detect the captured antigen. Assay signal is generated through a secondary antibody conjugated with horseradish peroxidase (HRP) such as a anti-His HRP. (FIGS. 45H-I) Viral antigen captured using multimeric ACE2 and the detection of the captured viral antigen is through a standard antibody as described in FIGS. 45F-G or by using multimeric antibodies (FIGS. 45J-K). A pair of non-competing multimeric antibodies used to capture and detect viral antigen (FIGS. 45L-M). Standard serological assay using IgG antibody such as a anti-IgG to capture immunoglobulin in test samples. Viral specific antibodies are detected with the addition of viral protein and the detection signal is generated through a secondary antibody conjugated with HRP such as anti-His HRP (FIG. 45N). A multimeric protein such as Protein G either in a tetravalent (FIG. 45O) or octavalent (FIG. 45P) format is used to capture antibodies in test samples and the viral specific antibodies are detected with the additional of viral antigen in the experimental mix as described in FIG. 45N. Antibodies CR3022 and CR3014 is used as an example of non-competing antibodies against SARS-CoV-2 spike protein. The viral detection or serological test could utilize any non-competing antibodies or protein molecules.

FIG. 46. CR3022 is a derivative of a human antibody isolated from a patient who recovered from SARS-CoV-1 infection. This antibody is known to bind the receptor binding domain (RBD) of CoV-1 strongly. It also cross-reacts with the RBD of CoV-2 strain but with significantly lower binding affinity as reflected in the above data. However Quad version of CR3022 massively improves the cross-reactive binding to RBD of CoV-2 and well as improving binding to the RBD domain CoV-1 strain. This makes the mulimter version of the CR3022 binding site a favorable candidate for developing as a novel SARS-CoV-2 neutralizing biologic compared to Ig antibodies. The mulimter version of the CR3022 binding site comprised 4 copies of the VH/VL antigen binding site of CR3022, as opposed to the 2 binding sites on the CR3022 Ig.

FIG. 47. Spike glycoprotein is naturally assembled into a trimer. It can be produced recombinantly as a trimer or only the RBD domain can be produced as a monomer for use in in vitro assays. The spike protein binds ACE2 receptor as the initial step in the infection process. Thus, ACE2 can be potentially used as a decoy receptor to neutralize coronavirus. In this experiment, an octavalent ACE2 Ig-TD Quad (ie, an example of a multimer comprising more than 2 binding sites; this example had 8 ACE2 extracellular proteins) was generated and used to capture the two recombinant forms of the spike protein for comparision. Strikingly, ACE2 Ig-TD Quad binds the trimeric form, which is more analogous to the native form with significantly enhanced binding strength than the RBD domain alone suggesting ACE2 Ig-TD is a good candidate for developing as a super neutralizer of SARS-CoV-2.

FIG. 48. As shown in the previous FIG. 47, ACE2 Ig-TD can bind spike trimer with higher binding affinity than the RDB domain monomer. The captured recombinant spike proteins were used here to capture seroconverted human antibodies in serum (i.e. The fluid and solute component of blood, which does not play a role in clotting, as the skilled addressee knows). The ACE2 Ig-TD captured trimer can detect seroconverted SARS-CoV-2 antibodies in serum with higher sensitivity than ACE2 Ig-TD captured RBD domain monomer. It is therefore anticipated that ACE2 Ig-TD can not only be used as a decoy to potently neutralize SARS-CoV-2 for therapeutic or prophylactic medical use, but it can alternatively be used in sensitive serological assays for detecting SARS-CoV-2 specific seroconverted antibodies.

FIG. 49. Schematic of Monomeric Polypeptide Chain Building Blocks useful to produce the multimeric structure of Quad formats.

FIGS. 50A-52C. Assay formats, reagents (and polypeptides of the invention useful for such assay reagents).

FIGS. 53A-D. Schematic representations of different Quad formats based on the Fab fragment of an IgG antibody. The monomeric building block of each Quad is shown with regions where optional linkers could be included are indicated. The fully assembled tetramer is also schematically represented in each example. The * denotes hinge region devoid of core hinge allowing for the production of monomeric Ig antibody formats.

FIGS. 54A-E. Schematic representation of ACE2 based Quads with varying valency from monovalent to octavelency either with or with Fc. In each format, the monomeric building block is shown with regions where optional linker could be included. The fully assembled tetramer is also schematically represented in each example. The * denotes hinge region devoid of core hinge allowing for the production of monomeric Ig formats.

FIGS. 55A-L. A schematic representations of Quad formats using VHH single domain antibodies as the binding domain. These formats can also be generated using other binding domains such as scFv’s, peptides and other protein domains and fragments. In each example, the monomeric building block is shown with regions where optional linker could be included. The fully assembled tetramer is also schematically represented in each example. The * denotes hinge region devoid of core hinge allowing for the production of monomeric Ig formats.

FIGS. 56A-J. Analysis of CR3022, CR3022-based Quads and ACE2 Ig-TD Quad for binding and neutralization of spike protein. (A-D) Schematic representation of the Quad formats from monomeric building blocks. Optional linker location and presence or absence of hinge region are indicated (E-F) Indirect binding ELISA of CR3022 and CR3022-based Quads to CoV-1 and CoV-2 RBD. (G) Competitive ELISA analyzing the ability of CR3022 Quads to inhibit ACE2:spike trimer interaction. (H). Analysis of ACE2 Ig-TD Quad binding to SARS CoV-2 full length spike trimer or CoV-2 RBD. (I) Competitive ELISA comparing ACE2:CoV-1 RBD and ACE2:CoV-2 RBD interaction inhibition using ACE2 Ig-TD. (J). Competitive ELISA comparing the neutralization potency for inhibiting ACE2:CoV-2 RBD interaction using CR3022 based Quads and ACE2 Ig-TD.

FIG. 57. Production and characterization of binding and neutralization potency of SARS CoV-2 reactive GB, BG and FE VHH-based Quads. (A-I) Schematic representation of the different GB Quad formats and (J) confirmation of the protein produced as analysed by SDS-PAGE. (K) Indirect binding ELISA showing binding of the different GB Quad proteins to SARS CoV-2 RBD and (L-N) their functional activity to neutralize ACE2:CoV-2 RBD interaction as analysed by competitive ELISA. (O-P) Reformating of BG and FE nanobodies into monomeric Ig Quad format schematically represented and (Q) their expression confirmed by SDS-PAGE. (R) Analysis of BG and FE Quad binding to CoV-2 RBD and (S) their functional activity to inhibit ACE2:CoV-2 RBD interaction as performed in a competitive ELISA.

FIG. 58. Competitive sandwich ELISA confirming extremely high neutralization potency of Q185 and Q186/Q182 as compared to potent neutralizing SARS-CoV-2 antibodies reported by Hansen et al (Hansen, J et al. Studies in humanized mice and convalescent humans yield a SARS-CoV-2 antibody cocktail. Science 15 Jun. 2020). A summary table comparing the apparent IC₅₀ of Q185 and Q186/Q182 and the lead neutralizing antibodies isolated by Hansen et al., are shown.

FIG. 59: Construction and analysis of multivalent monospecific and bispecific tandem VHH Quads. The tandem VHH multivalent Quads are schematically represented (A-F). The location of optional linker(s) and the hinge region status are shown for each Quad format as well as the dimeric intermediate structure for those Quads with an intact core hinge region. SDS-PAGE analysis of the tandem VHH multivalent Quads using either non-reduced or reduced proteins (G). In vitro neutralization potency curves as a dose response of SARS-CoV-2 RBD interaction with ACE2 (H) and a summary of the IC₅₀ values and potency comparisons of the different tandem VHH multivalent Quad molecules vs clinical stage mAbs (I).

FIG. 60: Construction and analysis of multivalent monospecific and bispecific tandem VHH Quads with increase valances ranging from 12-24. The tandem VHH multivalent Quads are schematically represented (A-L). The location of optional linker(s) and the hinge region status are shown for each Quad format as well as the dimeric intermediate structure for those Quads with an intact core hinge region. SDS-PAGE analysis of the tandem VHH multivalent Quads using either non-reduced (M) or reduced (N) proteins. The content of the lanes in the SDS-PAGE in figures M & N are as follows: 1. Q203/Q205, 2. Q204/Q206, 3. Q177D, 4. Q177E, 5. Q185D, 6. Q185E, 7. Q209/Q182, 8. Q209/Q205, 9. Q210/Q205, 10. Q211/Q206, 11. Q212/Q205 and 12. Q213/Q206. In vitro neutralization potency curves of a selected panel of tandem VHH Quads compared to REGN10987 and a previously constructed multivalent anti-SARS-CoV-2 Quad (Q185B).

FIG. 61: Construction and analysis of clinical stage mAbs reformatted into three different multivalent Quad formats as schematically represented (A - C). The expression of the newly formatted Quads for the three different clinical stage mAbs were analysed by SDS-PAGE either as non-reduced or reduced proteins (D - F) and their neutralization potential was compared to the parental IgG format as analysed by in vitro competitive ELISA (G - I). The potency enhancement as fold improvement of the Quad formats compared to the parental mAbs and the percentage neutralization of SARS-CoV-2 Spike interaction with ACE2 is summarized as IC₅₀ values (J).

FIG. 62: Production and analysis of multivalent Nb-112 Quads. Schematic representation of Nb-112 based Quads in a (A) tetravalent and a (B) octavalent configuration. The native VHH Nb-112 nanobody is reformatted with the inclusion of the TD domain to form the monomeric building block from which the tetrameric Quad protein is assembled as a stable protein. The location of the optional flexible linker(s) are shown with an arrow. SDS-PAGE analysis of Q231-Q233 and protein yields from Protein A and Ni-NTA affinity purification is shown (C). Competitive ELISA showing dose-dependent inhibition of ACE2 interaction with SARS-CoV-2 RBD at 2 nM (D) or 150 pM (E) fixed concentration. The IC₅₀ values and percentage neutralization at the highest concentration is shown in the table in panels D-2 and E-2.

FIG. 63: Production and analysis of multivalent Nb-112 Quads with an intact hinge region. Schematic representation of Nb-112 based Quads with intact hinge region in a (A) tetravalent and a (B) octavalent configuration. The native VHH Nb-112 nanobody is reformatted with the inclusion of the TD domain to form the monomeric building block from which the Quad protein is assembled through a dimeric intermediate into a stable tetrameric protein. SDS-PAGE analysis of Q231-Q233, Q246 and Q247 proteins is shown (C). Competitive ELISA showing dose-dependent inhibition of ACE2 interaction with SARS-CoV-2 RBD at fixed 150 pM is shown in panel D-1 and the table in panels D-2 shows their IC₅₀ values.All polypeptide schematics and amino acid sequences herein are written N- to C-terminal. All nucleotide sequences herein are written 5′ to 3′.

### DETAILED DESCRIPTION

The invention relates to multimers such as tetramers of polypeptides and tetramers, octamers, dodecamers, hexadecamers or 20-mesr (eg, tetramers and octamers) of epitopes or effector domains (such as antigen binding sites (eg, antibody or TCR binding sites that specifically bind to antigen or pMHC, or variable domains thereof)) or peptides such as incretin, insulin or hormone peptides. In embodiments, multimers of the invention are usefully producible in eurkaryotic systems and can be secreted from eukaryotic cells in soluble form, which is useful for various industrial applications, such as producing pharmaceuticals, diagnostics, as imaging agents, detergents etc. Higher order multimers, such as tetramers or octamers of effector domains or peptides are useful for enhancing antigen or pMHC binding avidity. This may be useful for producing an efficacious medicine or for enhancing the sensitivity of a diagnostic reagent comprising the multimer, such as tetramer or octamer. An additional or alternative benefit is enhanced half-life in vivo when the multimers of the invention are administered to a human or animal subject, eg, for treating or preventing a disease or condition in the subject. Usefully, the invention can also provide for multi-specific (eg, bi- or tri-specific) multivalent binding proteins. Specificity may related to specificity of antigen or pMHC binding. By using a single engineered polypeptide comprising binding domains or peptides, the invention in certain examples usefully provides a means for producing multivalent (eg, bi-specific) proteins at high purity. Use of a single species of engineered polypeptide monomer avoids the problem of mixed products seen when 2 or more different polypeptide species are used to produce multi- (eg, bi-) specific or multivalent proteins.

The invention also relates to methods and uses to expand antigen specificity of binding sites, as well as vaccines, methods of vaccination and assay methods and reagents.

The invention provides the following Clauses, Aspects, Paragraphs and Concepts (which are not intended to represent “Claims”; Claims are presented towards the end of this disclosure after the Examples and Tables). Any Clause herein can be combined with any Aspect or Concept herein. Any Aspect herein can be combined with any Concept herein.

### Aspects

The following Aspects are not to be interpreted as Claims. The Claims start after the Examples section.

1. A protein multimer of at least first, second, third and fourth copies of an effector domain (eg, a protein domain) or a peptide, wherein the multimer is multimerised by first, second, third and fourth self-associating tetramerisation domains (TDs) which are associated together, wherein each tetramerisation domain is comprised by a respective engineered polypeptide comprising one or more copies of said protein domain or peptide.

In an example, each TD is a TD of any one of proteins 1 to 119 listed in Table 2. In an example, each TD is a p53 TD or a homologue or orthologue thereof. In an example, each TD is a NHR2 TD or a homologue or orthologue thereof. In an example, each TD is a p63 TD or a homologue or orthologue thereof. In an example, each TD is a p73 TD or a homologue or orthologue thereof. In an example, each TD is not a NHR2 TD. In an example, each TD is not a p53 TD. In an example, each TD is not a p63 TD. In an example, each TD is not a p73 TD. In an example, each TD is not a p53, 63 or 73 TD. In an example, each TD is not a NHR2, p53, 63 or 73 TD.

By being “associated together”, the TDs in Aspect 1 multimerise first, second, third and fourth copies of the engineered polypeptide to provide a multimer protein, for example, a multimer that can be expressed intracellulary in a eukaryotic or mammalian cell (eg, a HEK293 cell) and/or which can be extracellularly secreted from a eukaryotic or mammalian cell (eg, a HEK293 cell) and/or which is soluble in an aqueous medium (eg, a eukaryotic or mammalian cell (eg, a HEK293 cell) culture medium). Examples are NHR TD, p53 TD, p63 TD and p73 TD (eg, human NHR TD, p53 TD, p63 TD and p73 TD) or an orthologue or homologue thereof.

In an example, the TD is not a p53 TD (or homologue or orthologue thereof), eg, it is not a human p53 TD (or homologue or orthologue thereof). In an example, the TD is a NHR2 TD or a homologue or orthologue thereof, but excluding a p53 TD or a homologue or orthologue thereof. In an example, the TD is a human NHR2 TD or a homologue or orthologue thereof, but excluding a human p53 TD or a homologue or orthologue thereof. In an example, the TD is human NHR2. In an example, the amino acid sequence of the TD is at least 80, 85, 90, 95, 96, 97, 98 or 99% identical to the sequence of human NHR2. In an example, the domain or peptide is not naturally comprised by a polypeptide that also comprise a NHR2 TD.

In an example, all of the domains of the polypeptide are human.

The engineered polypeptide may comprise one or more copies of said domain or peptide N-terminal to a copy of said TD. Additionally or alternatively, the engineered polypeptide may comprise one or more copies of said domain or peptide C- terminal to a copy of said TD. In an example, the engineered polypeptide comprises a first said domain or peptide and a TD, wherein the first domain or peptide is spaced by at least 10, 20, 30, 40, 50, 60, 70, 80, 90, 100, 200, 300, 400, 500, 600, 700, 800, 900 or 1000 contiguous amino acids from the TD, wherein there is no further said domain or peptide between the first domain or peptide and the TD.

In an example, the multimer (eg, tetramer of said engineered polypeptide) comprises 4 (but no more than 4) TDs (eg, identical TDs) and 4, 8, 12 or 16 (but no more than said 4, 8, 12 or 16 respectively) copies of said domain or peptide. In an example, each TD and each said domain or peptide is human.

In an example, the multimer, tetramer, octamer, dodecamer, hexadecamer or 20-mer (eg, tetramer or octamer) comprises first, second, third and fourth identical copies of an engineered polypeptide, the polypeptide comprising a TD and one (but no more than one), two (but no more than two), or more copies of the said protein domain or peptide. For example, a tetramer of the epitope or effector domain has 4 identical copies of the polypeptide comprising a TD and each polypeptide has 1 such epitope or effector domain. For example, an octamer of the epitope or effector domain has 4 identical copies of the polypeptide comprising a TD and each polypeptide has 2 such epitope or effector domain. For example, a dodecamer of the epitope or effector domain has 4 identical copies of the polypeptide comprising a TD and each polypeptide has 3 such epitope or effector domain. For example, a hexadecamer of the epitope or effector domain has 4 identical copies of the polypeptide comprising a TD and each polypeptide has 4 such epitope or effector domain. For example, a 20-mer of the epitope or effector domain has 4 identical copies of the polypeptide comprising a TD and each polypeptide has 5 such epitope or effector domain. Generally, for example, a X-mer of the epitope or effector domain has 4 identical copies of the polypeptide comprising a TD and each polypeptide has X/4 such epitope or effector domain, where X= any multiple of 4, eg, 4, 8, 12, 16, 20, 24, 28 or 32.

In some embodiments, by requiring just one type of engineered polypeptide to form the multimer, eg, tetramer or octamer, of the invention, the invention advantageously provides a format that can be readily isolated in pure (or highly pure, ie >90, 95, 96, 97, 98 or 99% purity) format, as well as a method for producing such a format in pure (or highly pure) form. Purity is indicated by the multimer of the invention not being in mixture in a composition with any other multimer or polypeptide monomer, or wherein the multimer of the invention comprises >90, 95, 96, 97, 98 or 99% of species in a composition comprising the multimer of the invention and other multimers and/or polypeptide monomers which comprise the engineered polyeptide. Thus, mixtures of different types of polypeptide in these embodiments are avoided or minimised. This advantageously also provides, therefore, plurality of multimers (eg, a plurality of tetramers or octamers or dodecamers or hexadecamers) that comprise only one (and no more than one) type of engineered polypeptide, wherein the multimers are monospecific (but multivalent) for antigen binding, or alternatively bi- or multi-specific for antigen binding. Thus, the invention provides a plurality of multimers (eg, a plurality of tetramers or octamers or dodecamers or hexadecamers, each polypeptide being at least tetra-valent for antigen binding and (i) bi-specific (ie, capable of specifically binding to 2 different antigens) or (ii) mono-specific and at least tetravalent for antigen binding. Herein, where antigen binding is mentioned this can instead be pMHC binding when the domain is a TCR V domain. Advantageously, the plurality is in pure form (ie, not mixed with multimers (eg, tetramers or octamers or dodecamers or hexadecamers) that comprise more than one type of polypeptide monomer. In an example, the multimer comprises at least 2 different types of antigen binding site. In an example, the multimer is bi-specific, tri-specific or tetra-specific. In an example, the multimer has an antigen binding site or pMHC binding site valency of 4, 6, 8, 10 or 12, preferably 4 or 8.

In an example, a peptide MHC (pMHC) is a class I or class II pMHC.

By the term “specifically binds,” as used herein, eg, with respect to a domain, antibody or binding site, is meant a domain, antibody or binding site which recognises a specific antigen (or pMHC) with a binding affinity of 1 mM or less as determined by SPR

Target binding ability, specificity and affinity (KD (also termed Kd), K_{off} and/or Kₒₙ) can be determined by any routine method in the art, eg, by surface plasmon resonance (SPR). The term “KD”, as used herein, is intended to refer to the equilibrium dissociation constant of a particular binding site/ligand, receptor/ligand or antibody/antigen interaction. In one embodiment, the surface plasmon resonance (SPR) is carried out at 25° C. In another embodiment, the SPR is carried out at 37° C. In one embodiment, the SPR is carried out at physiological pH, such as about pH7 or at pH7.6 (eg, using Hepes buffered saline at pH7.6 (also referred to as HBS-EP)). In one embodiment, the SPR is carried out at a physiological salt level, eg, 150 mM NaCl. In one embodiment, the SPR is carried out at a detergent level of no greater than 0.05% by volume, eg, in the presence of P20 (polysorbate 20; eg, Tween-20™) at 0.05% and EDTA at 3 mM. In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022). In an example, the affinity (eg, of a VH/VL binding site) is determined using SPR by using any standard SPR apparatus, such as by Biacore™ or using the ProteOn XPR36™ (Bio-Rad®). The binding data can be fitted to 1:1 model inherent using standard techniques, eg, using a model inherent to the ProteOn XPR36™ analysis software.

In an example, a multimer, tetramer or octamer or dodecamer or hexadecamer or 20-mer of the invention is an isolated multimer, tetramer or octamer or dodecamer or hexadecamer or 20-mer. In an example, a multimer, tetramer or octamer of the invention consists of copies of said engineered polypeptide. Optionally the multimer, tetramer or octamer or dodecamer or hexadecamer or 20-mer of the invention comprises 4 or 8 or 12 or 16 or 20 but not more than 4 or 8 or 12 or 16 or 20 copies respectively of the engineered polypeptide.

By “engineered” is meant that the polypeptide is not naturally-occurring, for example the protein domain or peptide is not naturally comprised by a polypeptide that also comprises said TD.

Each said protein domain or peptide may be a biologically active domain or peptide (eg, biologically active in humans or animals), such as a domain that specifically binds to an antigen or peptide-MHC (pMHC), or wherein the domain is comprised by an antigen or pMHC binding site. In an alternative, the domain or peptide is a carbohydrate, glucose or sugar-regulating agent, such as an incretin or an insulin peptide. In an alternative, the domain or peptide is an inhibitor or an enzyme or an inhibitor of a biological function or pathway in humans or animals. In an alternative, the domain or peptide is an iron-regulating agent. Thus, in an example, each protein domain or peptide is selected from an antigen or pMHC binding domain or peptide; a hormone; a carbohydrate, glucose or sugar-regulating agent; an iron-regulating agent; and an enzyme inhibitor.
2. The multimer of any preceding Aspect, wherein the multimer is a tetramer, octamer, 12-mer, 16-mer or 20-mer (eg, a tetramer, octamer, 12-mer or 16-mer) of said domain or peptide.
3. The multimer of any Aspect 1 or 2, comprising a tetramer, octamer, 12-mer, 16-mer or 20-mer (eg, a tetramer, octamer, 12-mer or 16-mer) of an immunoglobulin superfamily binding site (eg, an antibody or TCR binding site, such as a scFv or scTCR).

The immunoglobulin superfamily (IgSF) is a large protein superfamily of cell surface and soluble proteins that are involved in the recognition, binding, or adhesion processes of cells. Molecules are categorized as members of this superfamily based on shared structural features with immunoglobulins (also known as antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system.

T-cell receptor (TCR) domains can be Vα (eg. paired with a Vβ), Vβ (eg. paired with a Vα), Vγ (eg, paired with a V8) or Vδ (eg, paired with a Vy).

4. The multimer of Aspect 3, wherein the binding site comprises a first variable domain paired with a second variable domain.

In a first example, the first and second variable domains are comprised by the engineered polypeptide. In another example, the first domain is comprised by the engineered polypeptide and the second domain is comprised a by a further polypeptide that is different from the engineered polypeptide (and optionally comprises a TD or is devoid of a TD).

In the alternative, the domains are constant region domains. In an alternative, the domains are FcAbs. In an alternative, the domains are non-Ig antigen binding sites or comprises by a non-Ig antigen binding site, eg, an affibody.

### Antigen Binding Sites & Effector Domains

In an example, the or each antigen binding site (or effector domain) is selected from the group consisting of an antibody variable domain (eg, a VL or a VH, an antibody single variable domain (domain antibody or dAb), a camelid VHH antibody single variable domain, a shark immunoglobulin single variable domain (NA V), a Nanobody™ or a camelised VH single variable domain); a T-cell receptor binding domain; an immunoglobulin superfamily domain; an agnathan variable lymphocyte receptor (J Immunol; 2010 Aug 1;185(3):1367-74; “Alternative adaptive immunity in jawless vertebrates; Herrin BR & Cooper M D.); a fibronectin domain (eg, an Adnectin™); an scFv; an (scFv)₂; an sc-diabody; an scFab; a centyrin and an antigen binding site derived from a scaffold selected from CTLA-4 (Evibody™); a lipocalin domain; Protein A such as Z-domain of Protein A (eg, an Affibody™ or SpA); an A-domain (eg, an Avimer™ or Maxibody™); a heat shock protein (such as and epitope binding domain derived from GroEI and GroES); a transferrin domain (eg, a trans-body); ankyrin repeat protein (eg, a DARPin™); peptide aptamer; C-type lectin domain (eg, Tetranectin™); human γ- crystallin or human ubiquitin (an affilin); a PDZ domain; scorpion toxin; and a kunitz type domain of a human protease inhibitor.

Further sources of antigen binding sites are variable domains and VH/VL pairs of antibodies disclosed in WO2007024715 at page 40, line 23 to page 43, line 23. This specific disclosure is incorporated herein by reference as though explicitly written herein to provide basis for epitope binding moieties for use in the present invention and for possible inclusion in claims herein.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain

The phrase “immunoglobulin single variable domain” or “antibody single variable domain” refers to an antibody variable domain (VH, VHH, VL) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid VHH immunoglobulin single variable domains. Camelid VHH sre immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are still considered to be “domain antibodies” according to the invention. As used herein “VH includes camelid VHH domains. NA V are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. 44, 656-665 (2006) and US20050043519A. CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001). Lipocalins are a family of extracellular proteins which transport small hydrophobic molecules such as steroids, bilins, retinoids and lipids. They have a rigid β-sheet secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), US7250297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1. Avimers™ are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556 - 1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins™) are derived from ankyrin which is a family of proteins that mediate attachment of integral membrane proteins to the cytoskeleton. A single ankyrin repeat is a 33 residue motif consisting of two a-helices and a β-turn. They can be engineered to bind different target antigens by randomising residues in the first a-helix and a β-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. Fibronectin is a scaffold which can be engineered to bind to antigen. Adnectins™ consist of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435- 444 (2005), US20080139791, WO2005056764 and US6818418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges -examples of microproteins include KalataBI and conotoxin and knottins. The microproteins have a loop which can be engineered to include upto 25 amino acids without affecting the overall fold of the microprotein. For further details of engineered knottin domains, see WO2008098796. Other epitope binding moieties and domains include proteins which have been used as a scaffold to engineer different target antigen binding properties include human γ-crystallin and human ubiquitin (affilins), kunitz type domains of human protease inhibitors, PDZ- domains of the Ras-binding protein AF-6, scorpion toxins (charybdotoxin), C-type lectin domain (tetranectins) are reviewed in Chapter 7 - Non-Antibody Scaffolds from Handbook of Therapeutic Antibodies (2007, edited by Stefan Dubel) and Protein Science 15: 14-27 (2006).

In an example, the or each antigen binding site (or effector domain) comprises a non-Ig scaffoled, eg, is selected from the group consisting of Affibodies, Affilins, Anticalins, Atrimers, Avimers, Bicycle Peptides, Cys-knots, DARpins, Fibronectin type III, Fyomers, Kunitz Domain, OBodies, Aptamers, Adnectins, Armadillo Repeat Domain, Beta-Hairpin mimetics and Lipocalins.
5. The multimer of any preceding Aspect, wherein each polypeptide comprises first and second copies of said protein domain or peptide, wherein the polypeptide comprises in (N- to C-terminal direction) (i) a first of said copies - TD - the second of said copies; (ii) TD - and the first and second copies; or (iii) said first and second copies - TD.
6. The multimer of any preceding Aspect, wherein the TDs are NHR2 TDs and the domain or peptide is not a NHR2 domain or peptide; or wherein the TDs are p53 TDs and the domain or peptide is not a p53 domain or peptide.
7. The multimer of any preceding Aspect, wherein the engineered polypeptide comprises one or more copies of a second type of protein domain or peptide, wherein the second type of protein domain or peptide is different from the first protein domain or peptide.

For example, the polypeptide comprises in N-terminal direction (i) P1- TD -P2; or (ii) TD -P1-P2, wherein P1=a copy of a domain or peptide of the first type (ie, the type of domain or peptide of the multimer of Aspect 1); and P2=a copy of a domain or peptide of said second type.
8. The multimer of any preceding Aspect, wherein the domains are immunoglobulin superfamily domains.
9. The multimer of any preceding Aspect, wherein the domain or peptide is an antibody variable or constant domain (eg, an antibody single variable domain), a TCR variable or constant domain, an incretin, an insulin peptide, or a hormone peptide.
10. The multimer of any preceding Aspect, wherein the multimer comprises first, second, third and fourth identical copies of a said engineered polypeptide, the polypeptide comprising a TD and one (but no more than one), two (but no more than two) or more copies of the said protein domain or peptide.
11. The multimer of any preceding Aspect, wherein the engineered polypeptide comprises an antibody or TCR variable domain (V1) and a NHR2 TD.
12. The multimer of Aspect 11, wherein the polypeptide comprises (in N- to C-terminal direction) (i) V1-an optional linker-NHR2 TD; (ii) V1-an optional linker-NHR2 TD-optional linker-V2; or (iii) V1-an optional linker-V2 - optional linker - NHR2 TD, wherein V1 and V2 are TCR variable domains and are the same or different, or wherein V1 and V2 are antibody variable domains and are the same or different.
13. The multimer of Aspect 12, wherein V1and V2 are antibody single variable domains.
14. The multimer of aspect 11, wherein each engineered polypeptide comprises (in N- to C-terminal direction) V1-an optional linker-NHR2 TD, wherein V1 is an antibody or TCR variable domain and each engineered polypeptide is paired with a respective second engineered polypeptide that comprises V2, wherein V2 is a an antibody or TCR variable domain respectively that pairs with V1 to form an antigen or pMHC binding site, and optionally one polypeptide comprises an antibody Fc, or comprises antibody CH1 and the other polypeptide comprises an antibody CL that pairs with the CH1.
15. The multimer of any preceding Aspect, wherein the TD comprises (i) an amino acid sequence identical to SEQ ID NO: 10 or 126 or at least 80% identical thereto; or (ii) an amino acid sequence identical to SEQ ID NO: 120 or 123 or at least 80% identical thereto.
16. The multimer of any preceding Aspect, wherein the multimer comprises a tetramer, octamer, 12-mer, 16-mer or 20-mer (eg, a tetramer, octamer, 12-mer or 16-mer; or a tetramer or octamer) of an antigen binding site of an antibody selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Keytruda™, Opdivo™, Gazyva™ and Obinutuzumab. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

For example, a said protein domain of the engineered polypeptide is a V domain (a VH or VL) of an antibody binding site of an antibody selected from said group, wherein the multimer comprises a further V domain (a VL or VH respectively) that pairs with the V domain of the engineered polypeptide to form the antigen binding site of the selected antibody. Advantageously, therefore, the invention provides tetramer, octamer, 12-mer, 16-mer or 20-mer (eg, a tetramer, octamer, 12-mer or 16-mer; or tetramer or octamer)of a binding site of said selected antibody, which beneficially may have improved affinity, avidity and/or efficacy for binding its cognate antigen or for treating or preventing a disease or condition in a human or animal wherein the multimer is administered thereto to bind the cognate antigen in vivo.

For example, the multimer, tetramer, octamer, 12-mer, 16-mer or 20-mer (eg, a tetramer, octamer, 12-mer or 16-mer; or tetramer or octamer) comprises 4 (or said X/4 as described above) copies of an antigen binding site of an antibody, wherein the antibody is adalimumab, sarilumab, dupilumab, bevacizumab (eg, AVASTIN™), cetuximab (eg, ERBITUX™), tocilizumab (eg, ACTEMRA™) or trastuzumab (HERCEPTIN™). In an alternative the antibody is an anti-CD38 antibody, an anti-TNFa antibody, an anti-TNFR antibody, an anti-IL-4Ra antibody, an anti-IL-6R antibody, an anti-IL-6 antibody, an anti-VEGF antibody, an anti-EGFR antibody, an anti-PD-1 antibody, an anti-PD-L1 antibody, an anti-CTLA4 antibody, an anti-PCSK9 antibody, an anti-CD3 antibody, an anti-CD20 antibody, an anti-CD138 antibody, an anti-IL-1 antibody. In an alternative the antibody is selected from the antibodies disclosed in WO2007024715 at page 40, line 23 to page 43, line 23, the disclosure of which is incorporated herein by reference.

A binding site herein may, for example, be a ligand (eg, cytokine or growth factor, eg, VEGF or EGFR) binding site of a receptor (eg, KDR or Flt). A binding site herein may, for example, be a binding site of Eyelea™, Avastin™ or Lucentis™, eg, for ocular or oncological medical use in a human or animal. When the ligand or antigen is VEGF, the mutlimer, tetramer or octamer may be for treatment or prevention of a caner or ocular condition (eg, wet or dry AMD or diabetic retinopathy) or as an inhibitor of neovascularisation in a human or animal subject.

17. An isolated tetramer, octamer, dodecamer, hexadecamer or 20-mer of a TCR binding site, insulin peptide, incretin peptide or peptide hormone; or a plurality of said tetramers, octamers, dodecamers, hexadecamers or 20-mers.

Several important peptide hormones are secreted from the pituitary gland. The anterior pituitary secretes three hormones: prolactin, which acts on the mammary gland; adrenocorticotropic hormone (ACTH), which acts on the adrenal cortex to regulate the secretion of glucocorticoids; and growth hormone, which acts on bone, muscle, and the liver. The posterior pituitary gland secretes antidiuretic hormone, also called vasopressin, and oxytocin. Peptide hormones are produced by many different organs and tissues, however, including the heart (atrial-natriuretic peptide (ANP) or atrial natriuretic factor (ANF)) and pancreas (glucagon, insulin and somatostatin), the gastrointestinal tract (cholecystokinin, gastrin), and adipose tissue stores (leptin). In an example, the peptide hormone of the invention is selected from prolactin, ACTH, growth hormone (somatotropin), vasopressin, oxytocin, glucagon, insulin, somatostatin, cholecystokinin, gastrin and leptin (eg, selected from human prolactin, ACTH, growth hormone, vasopressin, oxytocin, glucagon, insulin, somatostatin, cholecystokinin, gastrin and leptin).

In an example, the incretin is a GLP-1, GIP or exendin-4 peptide.

The invention provides, in embodiments, the following engineered multimers:-
An isolated tetramer, octamer, dodecamer, hexadecamer or 20-mer of an incretin.
An isolated tetramer, octamer, dodecamer, hexadecamer or 20-mer of an insulin peptide.
An isolated tetramer, octamer, dodecamer, hexadecamer or 20-mer of a GLP-1 (glucagon-like peptide-1 (GLP-1) peptide.
An isolated tetramer, octamer, dodecamer, hexadecamer or 20-mer of a GIP (glucose-dependent insulinotropic polypeptide) peptide.
An isolated tetramer, octamer, dodecamer, hexadecamer or 20-mer of an exendin (eg, exendin-4) peptide.
An isolated tetramer, octamer, dodecamer, hexadecamer or 20-mer of a peptide hormone.
An isolated tetramer, octamer, dodecamer, hexadecamer or 20-mer of a prolactin or prolactin peptide.
An isolated tetramer, octamer, dodecamer, hexadecamer or 20-mer of a ACTH or ACTH peptide.
An isolated tetramer, octamer, dodecamer, hexadecamer or 20-mer of a growth hormone or growth hormone peptide.
An isolated tetramer, octamer, dodecamer, hexadecamer or 20-mer of a vasopressin or vasopressin peptide.
An isolated tetramer, octamer, dodecamer, hexadecamer or 20-mer of an oxytocin or oxytocin peptide.
An isolated tetramer, octamer, dodecamer, hexadecamer or 20-mer of a glucagon or glucagon peptide.
An isolated tetramer, octamer, dodecamer, hexadecamer or 20-mer of a insulin or insulin peptide.
An isolated tetramer, octamer, dodecamer, hexadecamer or 20-mer of a somatostatin or somatostatin peptide.
An isolated tetramer, octamer, dodecamer, hexadecamer or 20-mer of a cholecystokinin or cholecystokinin peptide.
An isolated tetramer, octamer, dodecamer, hexadecamer or 20-mer of a gastrin or gastrin peptide.
An isolated tetramer, octamer, dodecamer, hexadecamer or 20-mer of a leptin or leptin peptide.
An isolated tetramer, octamer, dodecamer, hexadecamer or 20-mer of an antibody binding site (eg, a scFv or Fab).
An isolated tetramer, octamer, dodecamer, hexadecamer or 20-mer of a TCR binding site (eg, a scTCR).
An isolated tetramer, octamer, dodecamer, hexadecamer or 20-mer of a TCR Vα/Vβ binding site.
An isolated tetramer, octamer, dodecamer, hexadecamer or 20-mer of a TCR Vγ/Vδ binding site.
An isolated tetramer, octamer, dodecamer, hexadecamer or 20-mer of an antibody single variable domain binding site.
An isolated tetramer, octamer, dodecamer, hexadecamer or 20-mer of an FcAb binding site.

In an example of any of these tetramer, octamer, dodecamer, hexadecamer or 20-mers, the domain or peptide is human. In an example of any of these tetramer, octamer, dodecamer, hexadecamer or 20-mers, the tetramer, octamer, dodecamer, hexadecamer or 20-mer comprises a NHR2 TD (eg, a human NHR2). In an example of any of these tetramer, octamer, dodecamer, hexadecamer or 20-mers, the tetramer, octamer, dodecamer, hexadecamer or 20-mer comprises a p53 TD (eg, a human p53 TD). In an example of any of these tetramer, octamer, dodecamer, hexadecamer or 20-mers, the tetramer, octamer, dodecamer, hexadecamer or 20-mer comprises a p63 TD (eg, a human p63 TD). In an example of any of these tetramer, octamer, dodecamer, hexadecamer or 20-mers, the tetramer, octamer, dodecamer, hexadecamer or 20-mer comprises a p73 TD (eg, a human p73 TD). In an example of any of these tetramer, octamer, dodecamer, hexadecamer or 20-mers, the tetramer, octamer, dodecamer, hexadecamer or 20-mer comprises a tetramer of TDs (eg, human NHR2 TDs), whereby the domains or peptides form a multimer of 4 or 8 domains or peptides.

In an example, the plurality is pure, eg, is not in mixture with multimers of said binding site or peptide wherein the multimers comprise more than one type of polypeptide monomer.

18. The multimer, tetramer, octamer, dodecamer, hexadecamer or 20-mer of any preceding Aspect, wherein the mulitmer, tetramer, octamer, dodecamer, hexadecamer or 20-mer is
(a) soluble in aqueous solution (eg, an aqueous eukaryotic cell growth medium or buffer);
(b) secretable from a eukaryotic cell; and/or
(c) an expression product of a eukaryotic cell.

In an example the multimer, tetramer, octamer, dodecamer, hexadecamer or 20-mer is secretable from a HEK293T (or other eukaryotic, mammalian, CHO or Cos) cell in stable form as indicated by a single band at the molecular weight expected for said multimer, tetramer, octamer, dodecamer, hexadecamer or 20-mer on a PAGE gel using a sample of supernatant from such cells and detected using Western Blot.

19. A tetramer, octamer, dodecamer, hexadecamer or 20-mer (eg, atetramer or octamer) of
(a) TCR V domains or TCR binding sites, wherein the tetramer, octamer, dodecamer, hexadecamer or 20-mer is soluble in aqueous solution (eg, an aqueous eukaryotic cell growth medium or buffer);
(b) antibody single variable domains, wherein the tetramer, octamer, dodecamer, hexadecamer or 20-mer is soluble in aqueous solution (eg, an aqueous eukaryotic cell growth medium or buffer);
(c) TCR V domains or TCR binding sites, wherein the tetramer, octamer, dodecamer, hexadecamer or 20-mer is capable of being intracellularly and/or extracellularly expressed by HEK293 cells; or
(d) antibody variable domains (eg, antibody single variable domains), wherein the tetramer, octamer, dodecamer, hexadecamer or 20-mer is capable of being intracellularly and/or extracellularly expressed by HEK293 cells.

An example of the medium is SFMII growth medium supplemented with L-glutamine (eg, complete SFMII growth medium supplemented with 4 mM L-glutamine). In an example, the medium is serum-free HEK293 cell culture medium. In an example, the medium is serum-free CHO cell culture medium.

For example, a cell herein is a human cell, eg, a HEK293 cell (such as a HEK293T cell).
20. The multimer, tetramer, octamer, dodecamer, hexadecamer or 20-mer of any preceding Aspect, wherein the tetramer, octamer, dodecamer, hexadecamer or 20-mer is bi-specific for antigen or pMHC binding.
21. The multimer, tetramer, octamer, dodecamer, hexadecamer or 20-mer of any preceding Aspect, wherein the domains are identical.
22. The multimer, tetramer, octamer, dodecamer, hexadecamer or 20-mer of any preceding Aspect, wherein the multimer, tetramer, octamer, dodecamer, hexadecamer or 20-mer comprises eukaryotic cell glycosylation.

For example, the glycosylation is CHO cell glycosylation. For example, the glycosylation is HEK (eg, HEK293, such as HEK293T) cell glycosylation. For example, the glycosylation is Cos cell glycosylation. For example, the glycosylation is Picchia cell glycosylation. For example, the glycosylation is Sacchaaromyces cell glycosylation.
23. The multimer, tetramer, octamer, dodecamer, hexadecamer or 20-mer of Aspect 22, wherein the cell is a HEK293 cell.
24. A plurality of multimers, tetramer, octamer, dodecamer, hexadecamer or 20-mer of any preceding Aspect.
25. A pharmaceutical composition comprising the multimer(s), tetramer(s), octamer(s), dodecamer(s), hexadecamer(s) or 20-mer(s) of any preceding Aspect and a pharmaceutically acceptable carrier, diluent or excipient.
26. A cosmetic, foodstuff, beverage, cleaning product, detergent comprising the multimer(s), tetramer(s), octamer(s), dodecamer(s), hexadecamer(s) or 20-mer(s) of any one of Aspects 1 to 24.
27. A said engineered (and optionally isolated) polypeptide or a monomer (optionally isolated) of a multimer, tetramer, octamer, dodecamer, hexadecamer or 20-mer of any preceding Aspect.

The monomer is an engineered polypeptide as disclosed herein, comprising a said protein domain or peptide and further comprising a TD.

Optionally, the engineered polypeptide comprises (in N- to C-terminal direction) a variable domain (V1) - a constant domain (C) (eg, a CH1 or Fc) - optional linker - TD.

28. An engineered (and optionally isolated) engineered polypeptide (P1) which comprises (in N-to C-terminal direction):-
(a) TCR V1 -TCR C1 - antibody C (eg, CH, CH1 (such as IgG CH1) or CL (such as Cλ or a Cκ)) - optional linker - TD, wherein
   (i) V1 is a Vα and C1 is a Cα;
   (ii) V1 is a Vβ and C1 is a Cβ;
   (iii) V1 is a Vγ and C1 is a Cy; or
   (iv) V1 is a Vδ and C1 is a Cδ; or
(b) TCR V1 - antibody C (eg, CH, CH1 (such as IgG CH1) or CL (such as Cλ or a Cκ)) -optional linker - TD, wherein
   (i) V1 is a Vα;
   (ii) V1 is a Vβ;
   (iii) V1 is a Vy; or
   (iv) V1 is a Vδ;
   or
(c) antibody V1 - antibody C (eg, CH, CH1 (such as IgG CH1) or CL (such as Cλ or a Cκ)) -optional linker - TD, wherein
   (i) V1 is a VH; or
   (ii) V1 is a VL (eg, a Vλ, or a Vκ);
   or
(d) antibody V1 - optional antibody C (eg, CH, CH1 (such as IgG CH1) or CL (such as Cλ or a Cκ)) - antibody Fc (eg, an IgG Fc) - optional linker - TD, wherein
   (i) V1 is a VH; or
   (ii) V1 is a VL (eg, a Vλ, or a Vκ);
   or
(e) antibody V1 - antibody CL (eg, a Cλ or a Cκ) - optional linker - TD, wherein
   (i) V1 is a VH; or
   (ii) V1 is a VL (eg, a Vλ or a Vκ);
   or
(f) TCR V1 -TCR C1 - optional linker - TD, wherein
   (i) V1 is a Vα and C1 is a Cα;
   (ii) V1 is a Vβ and C1 is a Cβ;
   (iii) V1 is a Vγ and C1 is a Cy; or
   (iv) V1 is a Vδ and C1 is a Cδ.

In (a) or (b), in an example, the TCR V is comprised by an single chain TCR binding site (scTCR) that specifically binds to a pMHC, wherein the binding site comprises TCR V-linker -TCRV. In an example, the engineered polypeptide comprises (in N- to C-terminal direction) (i) V1 -linker - V - optional C - optional linker - TD, or (ii) Va - linker - V1 - optional C - optional linker -TD, wherein Va is a TCR V domain and C is an antibody C domain (eg, a CH1 or CL) or a TCR C.

Preferably, the antibody C is CH1 (eg, IgG CH1).

In an example the multimer, tetramer, octamer, dodecamer, hexadecamer or 20-mer has a size of no more than 155 kDa, eg, wherein said protein domain is an antibody variable domain comprising a CDR3 of at least 16, 17, 18, 19, 20, 21 or 22 amino acids, such as a Camelid CDR3 or bovine CDR3.

In an example, the multimer, tetramer, octamer, dodecamer, hexadecamer or 20-mer comprises TCR binding sites and antibody binding sites. For example, each polypeptide comprises a TCR V (eg, comprised by a scTCR that specifically binds a pMHC) and an antibody V (eg, comprised by a scFv or paired with a second V domain comprised by a said second polypeptide to form a V/V paired binding site that specifically binds to an antigen). In an example, the pMHC comprises a RAS peptide. In an example the antigen is selected from the group consisting of PD-1, PD-L1 or any other antigen disclosed herein. For example, the antigen is PD-1 and the pMHC comprises a RAS peptide.

29. The polypeptide of Aspect 28, wherein the engineered polypeptide P1 is paired with a further polypeptide (P2), wherein P2 comprises (in N- to C-terminal direction):-
(g) TCR V2 -TCR C2 - antibody CL (eg, a Cλ or a Cκ), wherein P1 is according to (a) recited in Aspect 28 and
   (i) V2 is a Vα and C2 is a Cα when P1 is according to (a)(ii);
   (ii) V2 is a Vβ and C2 is a Cβ when P1 is according to (a)(i);
   (iii) V2 is a Vγ and C2 is a Cγ when P1 is according to (a)(iv); or
   (iv) V2 is a Vδ and C2 is a Cδ when P1 is according to (a)(iii);
   or
(h) TCR V2 - antibody CL (eg, a Cλ or a Cκ), wherein P1 is according to (b) recited in Aspect 28 and
   (i) V2 is a Vα when P1 is according to (b)(ii);
   (ii) V2 is a Vβ when P1 is according to (b)(i);
   (iii) V2 is a Vγ when P1 is according to (b)(iv); or
   (iv) V2 is a Vδ when P1 is according to (b)(iiii);
   or
(i) Antibody V2 - CL (eg, a Cλ or a Cκ), wherein P1 is according to (c) recited in Aspect 28 and
   (i) V2 is a VH when P1 is according to (c)(ii); or
   (ii) V2 is a VL (eg, a Vλ or a Vκ) when P1 is according to (c)(i);
   or
(j) Antibody V2 - optional CL (eg, a Cλ or a Cκ), wherein P1 is according to (d) recited in Aspect 28 and
   (i) V2 is a VH when P1 is according to (d)(ii); or
   (ii) V2 is a VL (eg, a Vλ or a Vκ) when P1 is according to (d)(i);
   or
(k) Antibody V2 - CH1 (eg, IgG CH1), wherein P1 is according to (e) recited in Aspect 28 and
   (i) V2 is a VH when P1 is according to (e)(ii); or
   (ii) V2 is a VL (eg, a Vλ or a Vκ) when P1 is according to (e)(i);
   or
(1) TCR V2 -TCR C2, wherein Pis according to (f) recited in Aspect 28 and
   (i) V2 is a Vα and C2 is a Cα when P1 is according to (f)(ii);
   (ii) V2 is a Vβ and C2 is a Cβ when P1 is according to (f)(i);
   (iii) V2 is a Vγ and C2 is a Cγ when P1 is according to (f)(iii); or
   (iv) V2 is a Vδ and C2 is a Cδ when P1 is according to (f)(iv).

Optionally, V1 and V2 form a paired variable domain binding site that is capable of specifically binding to an antigen or pMHC. In an example, V1 and V2 are variable domains of an antibody, eg, selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Keytruda™, Opdivo™, Gazyva™ and Obinutuzumab. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

In one embodiment, the antibody is Avastin.

In one embodiment, the antibody is Actemra.

In one embodiment, the antibody is Erbitux.

In one embodiment, the antibody is Lucentis.

In one embodiment, the antibody is sarilumab.

In one embodiment, the antibody is dupilumab.

In one embodiment, the antibody is alirocumab.

In one embodiment, the antibody is bococizumab.

In one embodiment, the antibody is evolocumab.

In one embodiment, the antibody is pembrolizumab.

In one embodiment, the antibody is nivolumab.

In one embodiment, the antibody is ipilimumab.

In one embodiment, the antibody is remicade.

In one embodiment, the antibody is golimumab.

In one embodiment, the antibody is ofatumumab.

In one embodiment, the antibody is Benlysta.

In one embodiment, the antibody is Campath.

In one embodiment, the antibody is rituximab.

In one embodiment, the antibody is Herceptin.

In one embodiment, the antibody is durvalumab.

In one embodiment, the antibody is daratumumab.

In an example, any binding domain herein (eg, a dAb or scFv or Fab) or V1 is capable (itself when a single variable domain, or when paired with V2) of specifically binding to an antigen selected from the group consisting of ABCF1; ACVR1; ACVR1B; ACVR2; ACVR2B; ACVRL1; ADORA2A; Aggrecan; AGR2; AICDA; AWI; AIG1; AKAP1; AKAP2; AIYIH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLRl (MDR15); BlyS; BM Pl; BMP2; BMP3B (GDFIO); BMP4; BMP6; BM P8; BMPRIA; BMPRIB; BM PR2; BPAG1 (plectin); BRCA1; CI9orflO (IL27w); C3; C4A; C5; C5R1; CANT1; CASP1; CASP4; CAV1; CCBP2 (D6 / JAB61); CCL1 (1-309); CCL11 (eotaxin); CCL13 (MCP-4); CCL15 (MIP-id); CCL16 (HCC-4); CCL17 (TARC); CCL18 (PARC); CCL19 (M IP-3b); CCL2 (MCP-1); MCAF; CCL20 (MIP-3a); CCL21 (MIP-2); SLC; exodus-2; CCL22 (MDC / STC-1); CCL23 (M PIF-1); CCL24 (MPIF-2 I eotaxin-2); CCL25 (TECK); CCL26 (eotaxin-3); CCL27 (CTACK /ILC) ; CCL28; CCL3 (MIP-la); CCL4 (M IP-lb); CCL5 (RANTES); CCL7 (MCP-3); CCL8 (mcp-2); CCNA1; CCNA2; CCND1; CCNE1; CCNE2; CCR1 (CKR1 / HM145); CCR2 (mcp-1RB / RA);CCR3 (CKR3 / CMKBR3); CCR4; CCR5 (CM KBR5 / ChemR13); CCR6 (CMKBR6 / CKR-L3 / STRL22 / DRY6); CCR7 (CKR7 / EBI1); CCR8 (CM KBR8 / TER1 / CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p2IWapl/Cipl); CDKN1B (p27Kipl); CDKNIC; CDKN2A (pl6INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYDi) ; CX3CR1 (V28); CXCL1 (GRO1); CXCLIO (IP-10); CXCL11 (1-TAC / IP-9); CXCL12 (SDF1); CXCL13; CXCL14; CXCL16; CXCL2 (GR02); CXCL3 (GR03); CXCL5 (ENA-78 I LIX); CXCL6 (GCP-2); CXCL9 (MIG); CXCR3 (GPR9/CKR-L2); CXCR4; CXCR6 (TYMSTRISTRL33 I Bonzo); CYB5; CYC1; CYSLTR1; DAB2IP; DES; DKFZp451J0118; DNCL1; DPP4; E2F1; ECGF1; EDG1; EFNAI; EFNA3; EFNB2; EGF; EGFR; ELAC2; ENG; EN01; EN02; EN03; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FU12584; FU25530; FLRT1 (fibronectin); FLT1; FOS; FOSL1 (FRA-I); FY (DARC); GABRP (GABAa); GAGEB1; GAGEC1; GALNAC4S-65T; GATA3; GDF5; GFI1; GGT1; GM-CSF; GNAS1; GNRH1; GPR2 (CCRIO); GPR31; GPR44; GPR81 (FKSG80); GRCCIO (CIO); GRP; GSN (Gelsolin); GSTP1; HAVCR2; HDAC4; EDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; 1D2; IFN-a; IFNA1; IFNA2; IFNA4; IFNA5; IFNA6; IFNA7; IFNB1; IFNgamma; TFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; 1L13; IL13RA1; IL13RA2; 1L14; 1L15; IL15RA; IL16; 1L17; IL17B; IL17C; IL17R; 1L18; IL18BP; IL18R1; IL18RAP; 1L19; ILIA; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1;IL1RL2 IL1RN; 1L2; 1L20; IL20RA; IL21R; 1L22; 1L22R; 1L22RA2; 1L23; 1L24; 1L25; 1L26; 1L27; 1L28A; 1L28B; 1L29; IL2RA; IL2RB; IL2RG; 1L3; 1L30; IL3RA; 1L4; IL4R; 1L5; IL5RA; 1L6; IL6R; IL6ST (glycoprotein 130); 1L7; TL7R; 1L8; IL8RA; IL8RB; IL8RB; 1L9; IL9R; ILK; INHA; INHBA; INSL3; INSL4; IRAKI; IRAK2; ITGA1; ITGA2; 1TGA3; ITGA6 (a6 integrin); ITGAV; ITGB3; ITGB4 (b 4 integrin); JAG1; JAK1; JAK3; JUN; K6HF; KAI1; KDR; MTLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; M IB1; midkine; M IF; M IP-2; MK167 (Ki-67); MMP2; M MP9; MS4A1; MSMB; MT3 (metallothionectin-ifi); MTSS 1; M UC 1 (mucin); MYC; MYD88; NCK2; neurocan; NFKB 1; NFKB2; NGFB (NGF); NGFR; NgR-Lingo; NgR-Nogo66 (Nogo); NgR-p75; NgR-Troy; NM E1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p2IRac2); RARB; RGS1; RGS13; RGS3; RNF110 (ZNF144); ROB02; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINIA3; SERPINB5 (maspin); SERPINE1 (PAT-i); SERPINF1; SHBG; SLA2; SLC2A2; SLC33A1; SLC43A1; SLIT2; SPP1; SPRRIB (Spri); ST6GAL1; STAB1; STAT6; STEAP; STEAP2; TB4R2; TBX21; TCPIO; TDGF1; TEK; TGFA; TGFB1; TGFB1I1; TGFB2; TGFB3; TGFBI; TGFBR1; TGFBR2; TGFBR3; TH1L; THBS1(thrombospondin-1); THBS2; THBS4; THPO; TIE (Tie-i); T]MP3; tissue factor; TLRIO; TLR2; TLR3; TLR4; TLR5; TLR6; TLR7; TLR8; TLR9; TNF; TNF-a (also referred to herein as TNF alpha or TNFα); TNFAIP2 (B94); TNFAIP3; TNFRSF1 1A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF1 1 (TRANCE); TNFSF12 (AP03L); TNFSF13 (April); TNFSF13B; TNFSF14 (HVEM-L); TNFSF1 5 (VEGI); TNFSF1 8; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM 1; TPM2; TRADD; TRAF1; TRAF2; TRAF3; TRAF4; TRAF5; TRAF6; TREM 1; TREM2; TRPC6; TSLP; TWEAK; VEGF; VEGFB; VEGFC; versican; VHL C5; VLA-4; XCL1 (lymphotactin); XCL2 (SCM-lb); XCR1 (GPR5 / CCXCR1); YY1; and ZFPM2.

For example in any configuration of the invention, the multimer, octamer, dodecamer, hexadecamer or 20-mer specifically binds to first and second (eg, for an octamer, dodecamer, hexadecamer or 20-mer); optionally, first, second and third (eg, for a dodecamer, hexadecamer or 20-mer); or optionally, first, second, third and fourth (eg, for a hexadecamer or 20-mer); or optionally, first, second, third, fourth and fifth (eg, for a 20-mer) epitopes or antigens, each of which is selected from the group consisting of EpCAM and CD3; CD19 and CD3; VEGF and VEGFR2; VEGF and EGFR; CD138 and CD20; CD138 and CD40; CD20 and CD3; CD38 and CD138; CD38 and CD20; CD38 and CD40; CD40 and CD20; CD19 and CD20; CD-8 and IL-6; PDL-1 and CTLA-4; CTLA-4 and BTN02; CSPGs and RGM A; IGF1 and IGF2; IGF1 and/or 2 and Erb2B; IL-12 and IL-18; IL-12 and TWEAK; IL-13 and ADAM8; IL-13 and CL25; IL-13 and IL-lbeta; IL-13 and IL-25; IL-13 and IL-4; IL-13 and IL-5; IL-13 and IL-9; IL-13 and LHR agonist; IL-13 and MDC; IL-13 and MIF; IL-13 and PED2; IL-13 and SPRR2a; IL-13 and SPRR2b; IL-13 and TARC; IL-13 and TGF-beta; IL-1 alpha and IL-1 beta; MAG and RGM A; NgR and RGM A; NogoA and RGM A; OMGp and RGM A; RGM A and RGM B; Te38 and TNF alpha; TNF alpha and IL-12; TNF alpha and IL-12p40; TNF alpha and IL-13; TNF alpha and IL-15; TNF alpha and IL-17; TNF alpha and IL-18; TNF alpha and IL-1 beta; TNF alpha and IL-23; TNF alpha and M IF; TNF alpha and PEG2; TNF alpha and PGE4; TNF alpha and VEGF; and VEGFR and EGFR; TNF alpha and RANK ligand; TNF alpha and Blys; TNF alpha and GP130; TNF alpha and CD-22; and TNF alpha and CTLA-4

For example, the first epitope or antigen is selected from the group consisting of CD3; CD16; CD32; CD64; and CD89; and the second epitope or antigen is selected from the group consisting of EGFR; VEGF; IGF-1R; Her2; c-Met (aka HGF); HER3; CEA; CD33; CD79a; CD19; PSA; EpCAM; CD66; CD30; HAS; PSMA; GD2; ANG2; IL-4; IL-13; VEGFR2; and VEGFR3.

In an example, any binding domain herein (eg, a dAb or scFv or Fab) or V1 is capable (itself when a single variable domain, or when paired with V2) of specifically binding to an antigen selected from the group consisting of human IL-1A, IL-1β, IL-1RN, IL-6, BLys, APRIL, activin A, TNF alpha, a BMP, BMP2, BMP7, BMP9, BMP10, GDF8, GDF11, RANKL, TRAIL, VEGFA, VEGFB or PGF; optionally the multimer comprises a cytokine amino acid sequence (eg, C-terminal to a TD), such as IL-2 or an IL2-peptide; and the multimer, octamer, dodecamer, hexadecamer or 20-mer is for treating or preventing a cancer in a human subject. In an example the said effector or protein domain is capable of binding to such an antigen; optionally the multimer comprises a cytokine amino acid sequence (eg, C-terminal to a TD), such as IL-2 or an IL2-peptide; and the multimer, tetramer, octamer, dodecamer, hexadecamer or 20-mer is for treating or preventing a cancer in a human subject.

30. A multimer (eg, a dimer, trimer, tetramer, octamer, dodecamer, hexadecamer or 20-mer) of P1 as defined in Aspect 28; or of P1 paired with P2 as defined in Aspect 29; or a plurality of said multimers, optionally wherein the multimer is according to any one of aspects 1 to 24.

Preferably the multimer is a tetramer of the engineered polypeptide and/or effector domain. In one example, the plurality of tetramers are not in mixture with monomers, dimers or trimers of the polypeptide,

In one example the multimer, eg, tetramer, is a capable of specifically binding to two different pMHC.

31. A nucleic acid encoding an engineered polypeptide or monomer of any one of Aspects 27 to 29, optionally wherein the nucleic acid is comprised by an expression vector for expressing the polypeptide.

In an example, the nucleic acid is a DNA, optionally operably connected to or comprising a promoter for expression of the polypeptide or monomer. In another example the nucleic acid is a RNA (eg, mRNA).
32. A eukaryotic host cell comprising the nucleic acid or vector of Aspect 31 for intracellular and/or secreted expression of the multimer, tetramer, octamer, dodecamer, hexadecamer or 20-mer, engineered polypeptide or monomer of any one of Aspects 1 to 24.
33. Use of a nucleic acid or vector according to aspect 31 in a method of manufacture of protein multimers for producing intracellularly expressed and/or secreted multimers, wherein the method comprises expressing the multimers in and/or secreting the multimers from eukaryotic cells comprising the nucleic acid or vector.
34. Use of a nucleic acid or vector according to aspect 31 in a method of manufacture of protein multimers for producing glycosylated multimers in eukaryotic cells comprising the nucleic acid or vector.

Mammalian glycosylation of the invention is useful for producing medicines comprising or consisting of the multimers, tetramer, octamer, dodecamer, hexadecamer or 20-merof the invention for medical treatment or prevention of a disease or condition in a mammal, eg, a human. The invention thus provides such a method of use as well as the multimer, tetramer, octamer, dodecamer, hexadecamer or 20-merof the invention for this purpose. Similarly, intracellular and/or secreted expression in one or more host cells (or cell lines thereof) that are mammalian according to the invention is useful for producing such medicines. Particularly useful is such expression in HEK293, CHO or Cos cells as these are commonly used for production of medicaments.

In an embodiment, the invention comprises a detergent or personal healthcare product comprising a multimer, tetramer, octamer, dodecamer, hexadecamer or 20-merof the invention. In an embodiment, the invention comprises a foodstuff or beverage comprising a multimer, tetramer, octamer, dodecamer, hexadecamer or 20-merof the invention.

In an example, the multimer, monomer, dimer, trimer, tetramer, octamer, dodecamer, hexadecamer or 20-mer, polypeptide, composition, mixture, use or method of the present invention is for an industrial or domestic use, or is used in a method for such use. For example, it is for or used in agriculture, oil or petroleum industry, food or drink industry, clothing industry, packaging industry, electronics industry, computer industry, environmental industry, chemical industry, aeorspace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.
35. A mixture comprising (i) a eukaryotic cell line encoding an engineered polypeptide according to any one of Aspects 27 to 29; and (ii) multimers, tetramers, octamers, dodecamers, hexadecamers or 20-mersas defined in any one of Aspects 1 to 24.
36. The mixture of Aspect 35, wherein the cell line is in a medium comprising secretion products of the cells, wherein the secretion products comprise said multimers, tetramers, octamers, dodecamers, hexadecamers or 20-mers.
37. The multimer, tetramer, octamer, dodecamer, hexadecamer or 20-merof any one of aspects 1 to 24 for medical use.
38. A method producing
   (a) TCR V domain multimers, the method comprising the soluble and/or intracellular expression of TCR V-NHR2 TD or TCR V- p53 TD fusion proteins expressed in eukaryotic cells, the method optionally comprising isolating a plurality of said multimers;
   (b) antibody V domain multimers, the method comprising the soluble and/or intracellular expression of antibody V (eg, a single variable domain)-NHR2 TD or V- p53 TD fusion proteins expressed in eukaryotic cells, the method optionally comprising isolating a plurality of said multimers;
   (c) incretin peptide (eg, GLP-1, GIP or insulin) multimers, the method comprising the soluble and/or intracellular expression of incretin peptide-NHR2 TD or incretin peptide-p53 TD fusion proteins expressed in eukaryotic cells, such as HEK293T cells; the method optionally comprising isolating a plurality of said multimers; or
   (d) peptide hormone multimers, the method comprising the soluble and/or intracellular expression of peptide hormone-NHR2 TD or peptide hormone- p53 TD fusion proteins expressed in eukaryotic cells, such as HEK293T cells; the method optionally comprising isolating a plurality of said multimers.
39. Use of self-associating tetramerisation domains (TD) (eg, NHR2 TD, p53 TD, p63 TD or p73 TD or a homologue or orthologue thereof) in a method of the manufacture of a tetramer of polypeptides, for producing a higher yield of tetramers versus monomer and/or dimer polypeptides.
40. Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).
41. Use of self-associating tetramerisation domains (TD) (eg, NHR2 TD, p53 TD, p63 TD or p73 TD or a homologue or orthologue thereof) in a method of the manufacture of a tetramer of a polypeptide, for producing a plurality of tetramers that are not in mixture with monomers, dimers or trimers.
42. Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).
43. The use of any one of Aspects 39 to 42, wherein the yield of tetramers is at least 10, 20, 30, 40 or 50x the yield of monomers and/or dimers.
44. The use of any one of Aspects 39 to 43, wherein the ratio of tetramers produced : monomers and/or dimers produced in the method is at least 90:10 (eg, at least 95:5 or 98:2, or 99:1).
45. The use of any one of Aspects 39 to 44, wherein each monomer has a size of no more than 40, 35, 30, 25 or 20 kDa.
46. The use of any one of Aspects 39 to 45, wherein each tetramer has a size of no more than 200, 160, 155 or 150 kDa.
47. The use of any one of Aspects 39 to 46, wherein the method comprises expressing the tetramers from a eukaryotic cell line.
48. A multivalent heterodimeric soluble T cell receptor capable of binding pMHC complex comprising:
   (i) TCR extracellular domains;
   (ii) immunoglobulin constant domains; and
   (iii) an NHR2 multimerisation domain of ETO.
49. A multimeric immunoglobulin, comprising
   (i) immunoglobulin variable domains; and
   (ii) an NHR2 multimerisation domain of ETO.
50. A method for assembling a soluble, multimeric polypeptide, comprising:
   (a) providing a monomer of the said multimeric polypeptide, fused to an NHR2 domain of ETO;
   (b) causing multiple copies of said monomer to associate, thereby obtaining a multimeric, soluble polypeptide.

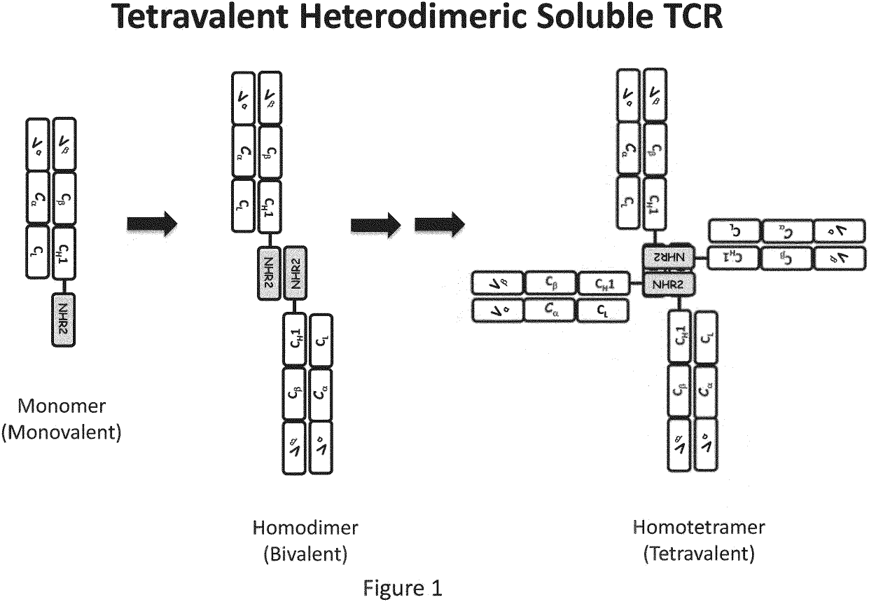

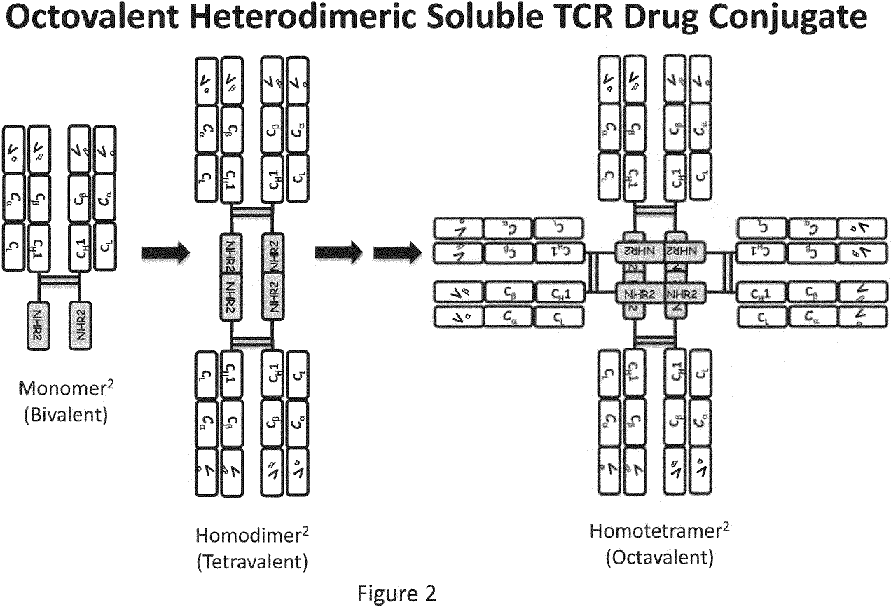

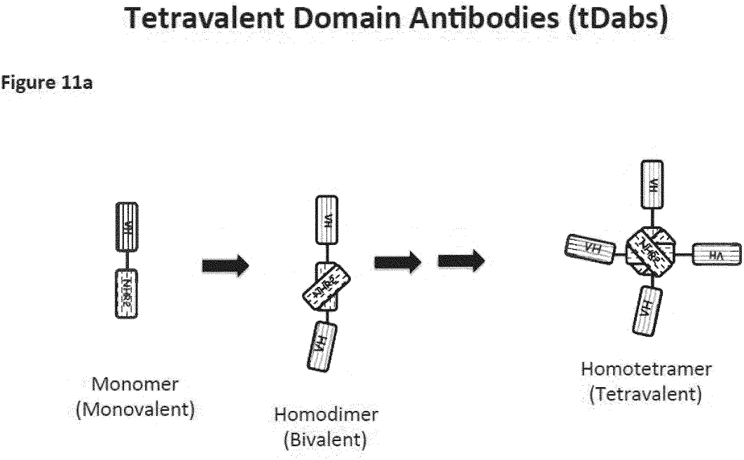

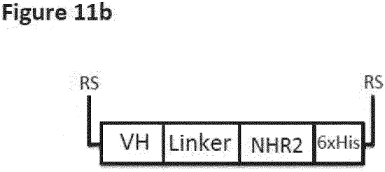

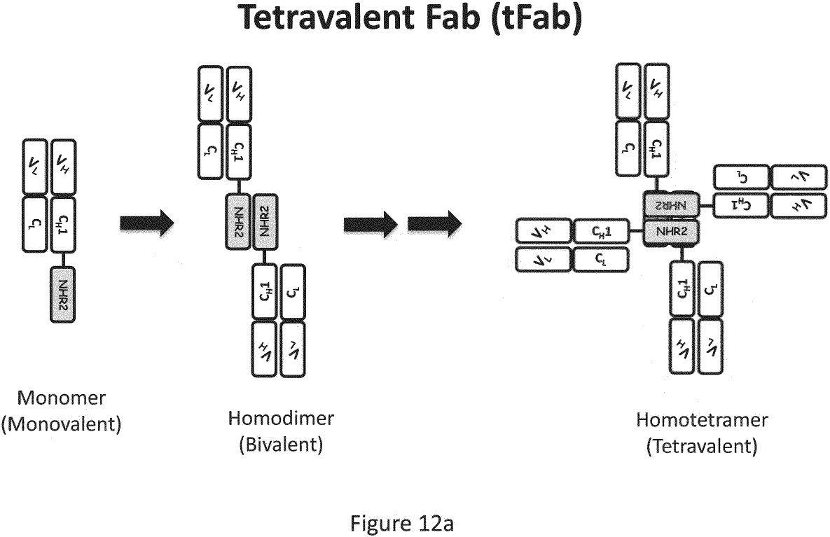

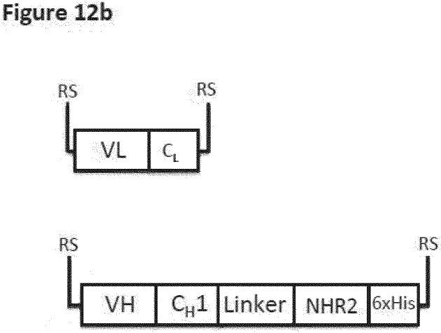

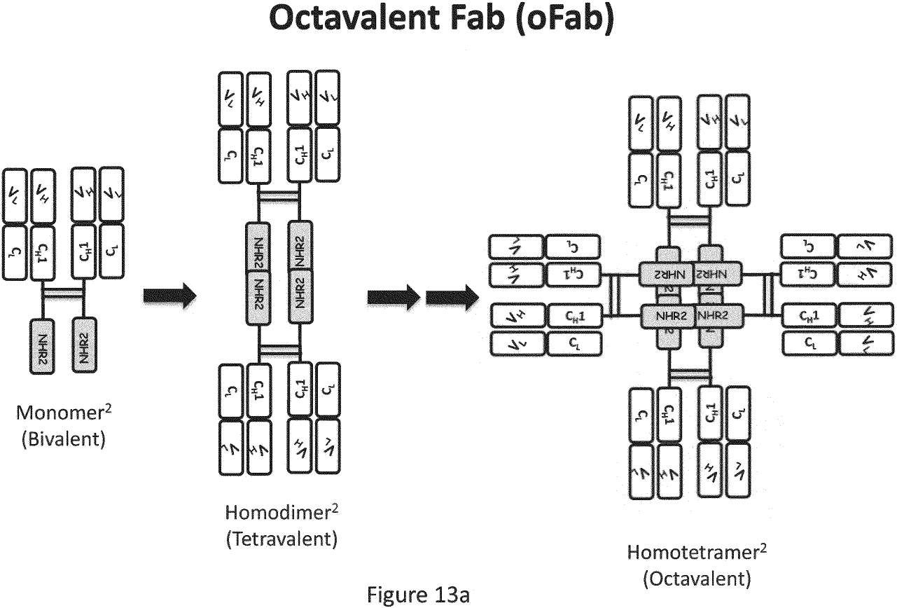

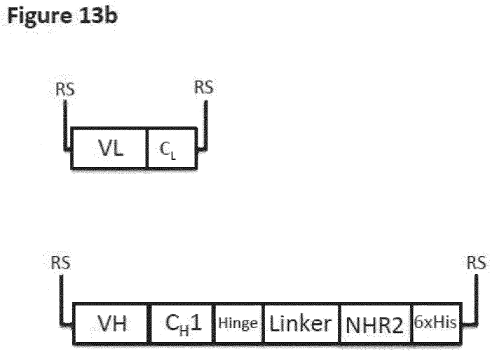

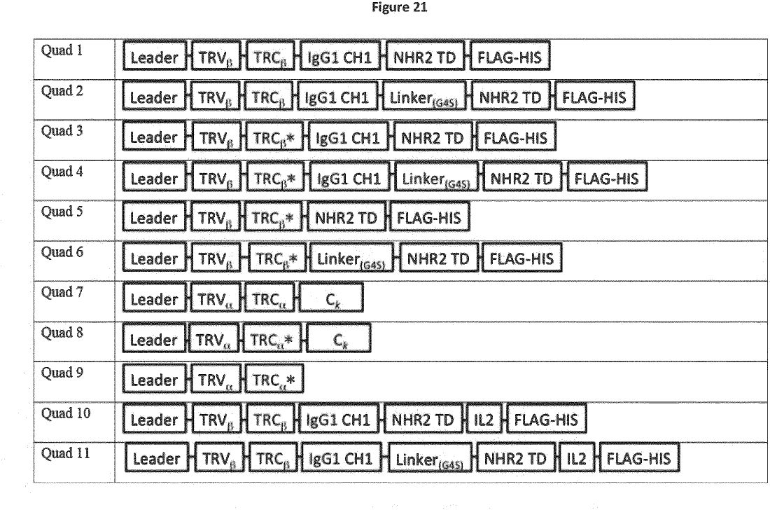

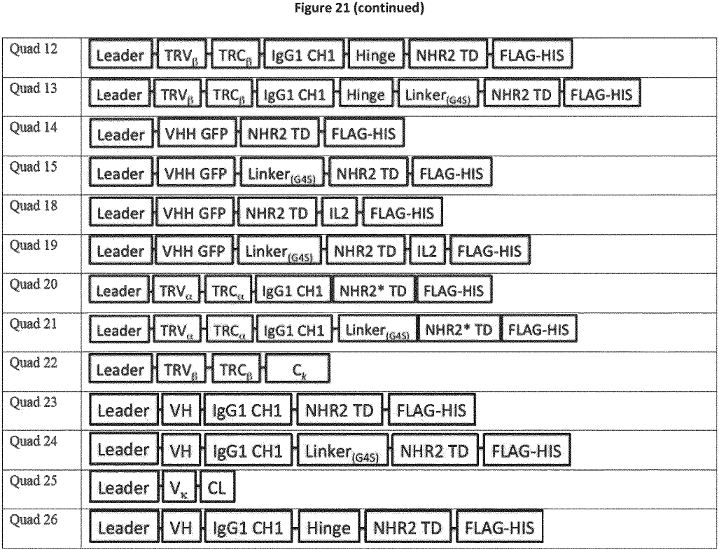

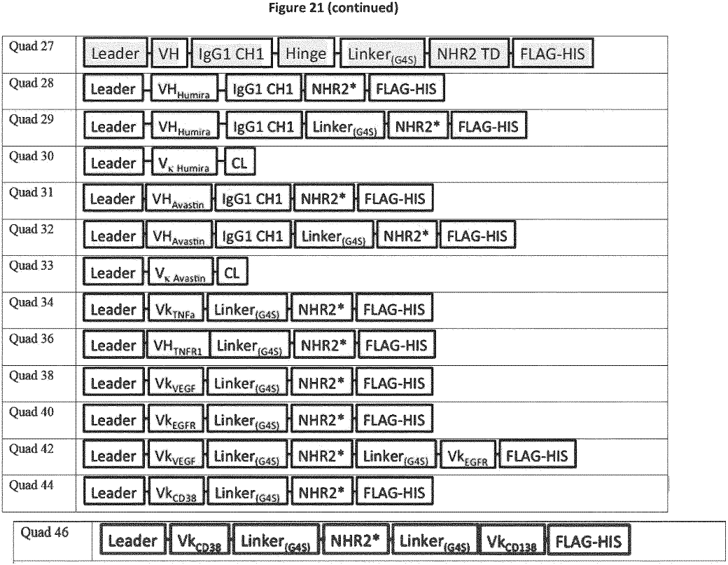

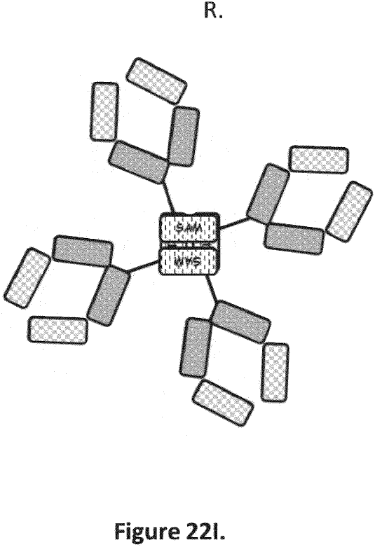

The invention further provides
(i) A monomer as shown in FIG. 1;
(ii) A homodimer as shown in FIG. 1;
(iii) A homotetramer as shown in FIG. 1;
(iv) A monomer² as shown in FIG. 2;
(v) A homodimer² as shown in FIG. 2;
(vi) A homotetramer² as shown in FIG. 2;
(vii) A monomer as shown in FIG. 11a;
(viii) A homodimer as shown in FIG. 11a;
(ix) A homotetramer as shown in FIG. 11a;
(x) A monomer as shown in FIG. 12a;
(xi) A homodimer as shown in FIG. 12a;
(xii) A homotetramer as shown in FIG. 12a;
(xiii) A monomer² as shown in FIG. 13a;
(xiv) A homodimer² as shown in FIG. 13a;
(xv) A homotetramer² as shown in FIG. 13a; or
(xvi) A multimeric protein comprising any one of (i) to (xv) (eg, any one of Quads 3, 4, 12, 13, 14, 15, 16 and 17) or a multimer of any protein shown in FIG. 21 (excluding any leader or tag);
(xvii) A plurality of multimers of (xvi); or
(xviii) A pharmaceutical composition comprising any one of (i) to (xvii) and a pharmaceutically acceptable carrier, diluent or excipient.

The invention also provides
(i) A tetravalent or octavalent antibody V molecule;
(ii) A tetravalent or octavalent antibody Fab molecule;
(iii) A tetravalent or octavalent antibody dAb molecule;
(iv) A tetravalent or octavalent antibody scFv molecule;
(v) A tetravalent or octavalent antibody TCR V molecule; or
(vi) A tetravalent or octavalent antibody scFv molecule;
Wherein the molecule is
(a) soluble in aqueous solution (eg, a solution or cell culture medium disclosed herein) and/or;
(b) capable of being intracellularly and/or extracellularly expressed by HEK293 cells.

The invention provides a claim multimer (eg, tetramer) of NHR2 or p53 (or another TD disclosed herein) fused at its N- and/or C-terminus to an amino acid sequence (eg, a peptide, protein domain or protein) that is not an NHR2 sequence. For example, sequence is selected from a TCR (eg, TCRα, TCRβ, Cα or Cβ), cytokine (eg, interleukin, eg, IL-2, IL-12, IL-12 and IFN), antibody fragments (eg, scFv, dAb or Fab) and a antibody domain (eg, V or C domain, eg, VH, VL, V_{K}, Vλ, CH, CH1, CH2, CH3, hinge, C_{K} or Cλ domain). Optionally, the multimer is the molecule is
a) soluble in aqueous solution (eg, a solution or cell culture medium disclosed herein) and/or;
b) capable of being intracellularly and/or extracellularly expressed by HEK293 cells.

The invention provides:-
(i) Use of NHR2 or p53 (or another TD disclosed herein) for the manufacture of a polypeptide for soluble expression of a multimer of the polypeptide from a cell, eg, a eukaryotic cell, eg, a mammalian, HEK293, CHO or Cos cell.
(ii) Use of NHR2 or p53 (or another TD disclosed herein) for the manufacture of a polypeptide for intracellular expression of a multimer of the polypeptide in a cell, eg, a eukaryotic cell, eg, a mammalian, HEK293, CHO or Cos cell.
(iii) A cell comprising an intracelllular expression product, wherein the product comprises a multimer of a polypeptide comprising NHR2 or p53 (or another TD disclosed herein) fused at its N- and/or C-terminus to an amino acid sequence (eg, a peptide, protein domain or protein) that is not an NHR2 sequence.
(iv) Use of NHR2 as a promiscuous tetramerisation domain for tetramerising peptides, protein domains, polypeptides or proteins in tha manufacture of multimers that are intracellularly and/or solubly expressed from host cell.

Optionally, the amino acid is an amino acid sequence of a human peptide, protein domain or protein,eg, a TCR (eg, TCRα, TCRβ, Cα or Cβ), cytokine (eg, interleukin, eg, IL-2, IL-12, IL-12 and IFN), antibody fragments (eg, scFv, dAb or Fab), or an antibody domain (eg, V or C domain, eg, VH, VL, V_{K}, Vλ, CH, CH1, CH2, CH3, hnige, C_{K} or Cλ domain).

Optionally, the or each polypeptide comprises a polypeptide selected from the group consisting of Quad 1-46 (ie, a polypeptide as shown in FIG. 21 but excluding any leader or tag sequence). Optionally, the invention provides a multimer (eg, a dimer, trimer, tetramer, pentamer, hexamer, septamer or octamer, preferably a tetramer or octamer) of a polypeptide selected from the group consisting of such Quad 1-46 (ie, 2, 3, 4, 5, 6, 7 or 8 copies of such a polypeptide), eg, for medical or diagnostic use, eg, medical use for treating or preventing a disease or condition in a human or animal (eg, a human).

Optionally, the or each polypeptide comprises a polypeptide (excluding any leader or tag sequence) that is encoded by a nucleotide sequence selected from the group consisting of SEQ ID NOs: 13-50. Optionally, the or each polypeptide comprises a polypeptide (excluding any leader or tag sequence) that comprises an amino acid sequence selected from the group consisting of SEQ ID NOs: 83-115. Optionally, the invention provides a multimer (eg, a dimer, trimer, tetramer, pentamer, hexamer, septamer or octamer, preferably a tetramer or octamer) of such a polypeptide, eg, for medical or diagnostic use, eg, medical use for treating or preventing a disease or condition in a human or animal (eg, a human).

In an example, the TD is a TD comprised by any one of SEQ ID NOs: 1-9. In an example, the TD is a TD comprising SEQ ID NO: 10 or 126. In an example, the TD is encoded by SEQ ID NO: 124 or 125. In an example, the amino acid sequence of each TD is SEQ ID NO: 10 or 126 or is at least 80, 85, 90, 95, 96m 97, 98 or 99% identical to SEQ ID NO: 10 or 126.

In an example, the TD is a TD comprising SEQ ID NO: 120 or 123. In an example, the TD is encoded by SEQ ID NO: 116 or 119. In an example, the amino acid sequence of each TD is SEQ ID NO: 120 or 123 or is at least 80, 85, 90, 95, 96m 97, 98 or 99% identical to the SEQ ID NO: 120 or 123.

Optionally, the domain or peptide comprised by the engineered polypeptide or monomer comprises an amino acid selected from SEQ ID NOs: 51-82.

### High Purity Tetramers

As exemplified herein, the invention in one configuration is based on the surprising realization that tetramerisation domains (TD), eg, p53 tetramerisation domain (p53 TD), can be used to preferentially produce tetramers of effector domains over the production of lower-order structures such as dimers and monomers. This is particularly useful for secretion of tetramers is desirable yields from mammalian expression cell lines, such as CHO, HEK293 and Cos cell lines. The invention is also particularly useful for the production of tetramers no more than 200, 160, 155 or 150 kDa in size.

Thus, the invention provides the following Concepts:-

### Concepts

The following Concepts are not to be interpreted as Claims. The Claims start after the Examples section.

1. Use of a tetramerisation domain (TD) (eg, p53 tetramerisation domain (p53 TD) or NHR2 TD) or a homologue or orthologue thereof in a method of the manufacture of a tetramer of polypeptides, for producing a higher yield of tetramers versus monomer and/or dimer polypeptides.

The monomers and dimers comprise one or two copies of the TD, homologue or orthologue respectively

In an example, the TD, orthologue or homologue is a human domain.

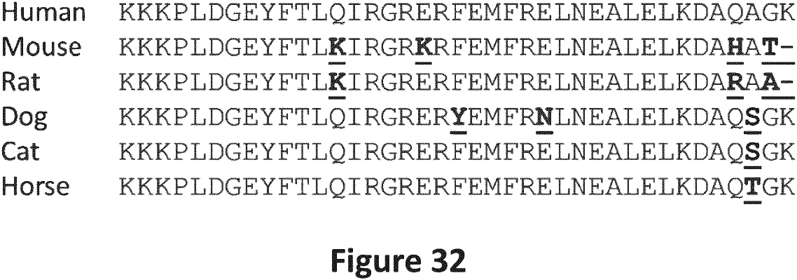

Herein, the TD is a human TD or a homologue, eg, a TD selected from any of the p53 TD sequences disclosed in UniProt (www.uniprot.org), for example the p53 TD is a TD disclosed in Table 13. In an example, the homologue is a p53TD of a non-human animal species, eg, a mouse, rat, horse cat or dog p53TD. See FIG. 32, which shows the high level of conservation between p53 TDs of different species, which supports the use of non-human p53 TDs as an alternative to human p53 TDs. In an example, the homologue is a p53TD of a non-human mammalian species. In an example, the homologue is identical to human p53 TD with the exception of up to 10, 9, 8, 7, 6, 5, 4, 3, 2 or 1 amino acid change(s).

In an example, the yield of tetramers is higher than the yield of monomers; In an example, the yield of tetramers is higher than the yield of dimers; In an example, the yield of tetramers is higher than the yield of trimers; In an example, the yield of tetramers is higher than the yield of monomers and dimers; In an example, the yield of tetramers is higher than the yield of monomers and trimers; In an example, the yield of tetramers is higher than the yield of monomers, dimers and trimers

For example, the TD is the TD of p53 isoform 1. In an example, the TD comprises or consists of an amino acid sequence that is identical to positions 325 to 356 (or 319-360; or 321-359) of human p53 (eg, isoform 1). Optionally, the TD, orthologue or homologue comprises or consists of an amino acid sequence that is at least 80, 85, 90, 95, 96, 97, 98 or 99% identical to SEQ ID NO: 10, 126, 11 or 12. For example the sequence is identical to said selected sequence. Optionally, the TD, orthologue or homologue comprises or consists of an amino acid sequence that is at least 80, 85, 90, 95, 96, 97, 98 or 99% identical to SEQ ID NO: 120, 121, 122 or 123. For example the sequence is identical to said selected sequence.
2. The use of Concept 1, wherein first, second, third and fourth copies of an identical TD or homologue or orthologue thereof is used.
3. The use of any preceding Concept, wherein the TD is a NHR2, p53, p63 or p73 tetramerisation domain.
For example, the TD is a p53 TD. In an example, the TD is an orthologue or homologue of a p53 TD, eg, a human p53 TD.
4. The use of any preceding Concept, wherein the yield of tetramers is at least 10x the yield of monomers and/or dimers.

Optionally, the yield is at least 2× 3×, 4×, 5×, 6×, 7×, 8×, 9×, or 10× the yield of monomers and/or dimers. Optionally, the ratio of tetramers produced : monomers and/or dimers is at least 90:10, eg, at least 95:5; or 96:4; or 97:3; or 98:2; or 99:1. Optionally only tetramers are produced.

In an embodiment, each domain comprised by each monomer, dimer or tetramer is a human domain; and optionally the monomer, dimer or tetramer does not comprise non-human amino acid sequences or linkers.
5. The use of any preceding Concept, wherein the ratio of tetramers produced : monomers and/or dimers produced in the method is at least 90:10 (ie, 9× the amount of monomers; 9× the amount of dimers; or 9× the amount of the combination of monomers and dimers).
6. The use of Concept 4 or 5, wherein the yield or ratio is determinable or determined by obtaining a sample of the product of the tetramer manufacture method, using a protein separation technique on the sample to resolve tetramers, monomers and dimers and comparing the amount of tetramers with the amount of monomers and dimers.

Amounts of tetramers, monomers, dimers and trimers can be determined, for example, using Western Blot analysis of a gel described herein, eg, a native gel, ie, a gel not under denatured conditions, such as in the absence of SDS.
7. The use of Concept 4 or 5, wherein the yield or ratio is determinable or determined by
   (a) Obtaining a sample of the product of the tetramer manufacture method;
   (b) Carrying out polyacrylamide gel electrophoresis (PAGE) under non-reducing conditions to resolve the sample into a band corresponding to said tetramers and a band corresponding to said monomers and/or a band corresponding to said dimers; and
   (c) Comparing the tetramer band with the monomer and/or dimer band(s) to determine said yield or ratio, eg, by comparing the relative band intensities and/or band sizes.
8. The use of Concept 4 or 5, wherein the yield or ratio is determinable or determined by
   (d) Obtaining a sample of the product of the tetramer manufacture method;
   (e) Carrying out polyacrylamide gel electrophoresis (PAGE) under non-reducing conditions to resolve the sample into a band corresponding to said tetramers, eg, wherein the gel is under non-denatured conditions (eg, in the absence of sodium dodecylsuphate (SDS);
   (f) Determining that there is no band corresponding to said monomers and/or no band corresponding to said dimers.
9. The use of Concept 7 or 8, comprising
   (g) Obtaining a second sample of the product of the tetramer manufacture method;
   (h) Carrying out polyacrylamide gel electrophoresis (PAGE) under reducing conditions to resolve the second sample into a band corresponding to said monomers and/or a band corresponding to said dimers, eg, wherein the gel is under non-denatured conditions (eg, in the absence of sodium dodecylsuphate (SDS); and
   (i) Comparing the gel produced by step (h) with the gel of step (b) or (e) to determine the position of monomer and/or dimer band(s) in the gel of step (b) or where such gels would be expected in the gel of step (e).
10. The use of any preceding Concept, wherein each monomer has a size of no more than 40 kDa.

For example, the monomer has a size of no more than 35, 30, 25, 24, 23, 22, 21 or 20 kDa

11. The use of any preceding Concept, wherein each tetramer has a size of no more than 150 kDa.

For example, the tetramer has a size of no more than 80, 90, 100, 110, 120, 130 or 140 kDa.

12. The use of any preceding Concept, wherein the method comprises expressing the tetramers from a mammalian cell line, eg, a HEK293, CHO or Cos cell line.

For example, the cell line is a HEK293 (eg, HEK293T) cell line. In the alternative, the cell line is a yeast (eg, Saccharomyces or Pichia, eg, P pastoris) or bacterial cell line.

13. The use of any preceding Concept, wherein the method comprises secreting the tetramers from a mammalian cell line, eg, a HEK293, CHO or Cos cell line.

Thus, advantageously in an example, the use or tetramer is for expression from a mammalian cell line (eg, a HEK293, CHO or Cos cell line) or a eukaryotic cell line. This is useful for large-scale manufacture of the products, eg, tetramers, of the invention.

For example, the cell line is a HEK293 (eg, HEK293T) cell line. In the alternative, the cell line is a yeast (eg, Saccharomyces or Pichia, eg, P pastoris) or bacterial cell line.
14. The use of any preceding Concept, wherein each polypeptide or monomer comprises a said TD, homologue or orthologue and one or more protein effector domains, such as one or more antibody domains, eg, one or more antibody domains forming an antigen binding site.
15. The use of Concept 14, wherein the polypeptide comprises one or more of
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
16. The use of any preceding Concept, wherein each polypeptide or monomer comprises a said TD, homologue or orthologue and one or more incretin, insulin, GLP-1 or Exendin-4 domains.
17. The use of any preceding Concept, wherein each polypeptide or monomer comprises a said TD, homologue or orthologue; and first and second antigen binding sites.
18. The use of Concept 17, wherein each binding site is provided by
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
19. The use of Concept 18, wherein each binding site is provided by an antibody single variable domain.
20. The use of any one of Concepts 14 to 18, wherein the TD, homologue or orthologue is directly fused to said further domain(s).
21. The use of Concept 20, wherein each monomer or polypeptide comprises the TD, homologue or orthologue fused directly or via a peptide linker to the C-terminal of a said further domain.
22. A tetramer of polypeptides, wherein each polypeptide comprises
   (i) a tetramerisation domain (TD) (eg, a p53 TD or a NHR2 TD) or a homologue or orthologue thereof;
   (ii) one or more protein effector domains; and
   (iii) optionally a linker linking (i) to (ii) (eg, linking the C-terminus of (ii) to the N-terminus of (i));
   wherein optionally each tetramer has a size of no more than 150 or 200 kDa.

For example, the tetramer has a size of no more than 80, 90, 100, 110, 120, 130 or 140 kDa. In an example, any multimer, dimer, trimer, tetramer, octamer, dodecamer, hexadecamer or 20-merherein has a size of at least 60 or 80 kDa; this may be useful for example to increase half -life in a human or animal subject administered with the multimer, dimer, trimer, tetramer, octamer, dodecamer, hexadecamer or 20-mer (eg, to treat or prevent a disease or condition in the subject). Sizes in these ranges may be above the renal filtration size.

In an alternative, the invention provides a monomer, dimer, octamer, dodecamer, hexadecamer or 20-merinstead of a tetramer.
23. The tetramer of Concept 22, wherein each polypeptide comprises one or more of
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
24. The tetramer of Concept 22 or 23, wherein each polypeptide comprises a said TD, homologue or orthologue and one or more incretin, insulin, GLP-1 or Exendin-4 domains.
25. The tetramer of Concept 22 or 23, wherein each polypeptide comprises a said TD, homologue or orthologue; and first and second antigen binding sites.
26. The tetramer of Concept 25, wherein each binding site is provided by (i) an antibody single variable domain (dAb or VHH or Nanobody™) that is capable of specifically binding an antigen;
(ii) an scFv that is capable of binding an antigen or an scTCR that is capable of binding pMHC;
(iii) a Fab that is capable of binding an antigen; or
(iv) a TCR variable domain or pMHC binding site.
27. The tetramer of Concept 26, wherein each binding site is provided by an antibody single variable domain.
28. The tetramer of any one of Concepts 22 to 27, wherein the TD, homologue or orthologue is directly fused to said effector domain(s).
29. The tetramer of any one of Concepts 22 to 27, wherein each polypeptide comprises the TD, homologue or orthologue fused directly or via a peptide linker to the C-terminal of a said effector domain.

In an embodiment, each polypeptide comprises only 2 (ie, only a first and a second, but not a third) effector domains or only 2 dAbs, VHH, scFvs, scTCRs, Fabs or antigen binding sites.

30. A pharmaceutical composition comprising a tetramer of any one of Concepts 22 to 29 and a pharmaceutically acceptable carrier, diluent or excipient.

Optionally the composition is comprised by a sterile medical container or device, eg, a syringe, vial, inhaler or injection device.
31. A cosmetic, foodstuff, beverage, cleaning product, detergent comprising a tetramer of any one of Concepts 22 to 29.
32. A mixture comprising a cell line (eg, a mammalian cell line, eg, a HEK293, CHO or Cos cell line) encoding a polypeptide as recited in any preceding Concept; and tetramers as defined in any preceding Concept.

Optionally, the mixture is comprised by a sterile container.
33. The mixture of Concept 32, wherein the cell line is in a medium comprising secretion products of the cells, wherein the secretion products comprise said tetramers.
34. The mixture of Concept 33, wherein the secretion products do not comprise monomers and/or dimers as defined in any one of Concepts 1 to 31.
35. The mixture of Concept 33, wherein the secretion products comprise said tetramers in an amount of at least 10× the amount of monomers and/or dimers.
36. The mixture of Concept 33, wherein the secretion products comprise said tetramers in a ratio of tetramers : monomers and/or dimers of at least 90:10.
37. A method for enhancing the yield of tetramers of an protein effector domain (eg, an antibody variable domain or binding site), the method comprising expressing from a cell line (eg, a mammalian cell, CHO, HEK293 or Cos cell line) tetramers of a polypeptide, wherein the polypeptide is as defined in any preceding Concept and comprises one or more effector domains; and optionally isolating said expressed tetramers.

The homologue, orthologue or equivalent has multimerisation or tetramerisation function.

Homologue: A gene, nucleotide or protein sequence related to a second gene, nucleotide or protein sequence by descent from a common ancestral DNA or protein sequence. The term, homologue, may apply to the relationship between genes separated by the event of or to the relationship between genes separated by the event of genetic duplication.

Orthologue: Orthologues are genes, nucleotide or protein sequences in different species that evolved from a common ancestral gene, nucleotide or protein sequence by speciation. Normally, orthologues retain the same function in the course of evolution.

In an example, the TD, orthologue or homologue is a TD of any one of proteins 1 to 119 listed in Table 2. In an example, the orthologue or homologue is an orthologue or homologue of a TD of any one of proteins 1 to 119 listed in Table 2. In an embodiment, instead of the use of a p53 tetramerisation domain (p53-TD) or a homologue or orthologue thereof, all aspects of the invention herein instead can be read to relate to the use or inclusion in a polypeptide, monomer, dimer, trimer or tetramer of aTD of any one of proteins 1 to 119 listed in Table 2 or a homologue or orthologue thereof. The TD may be a NHR2 (eg, a human NHR2) TD or an orthologue or homologue thereof. The TD may be a p63 (eg, a human p63) TD or an orthologue or homologue thereof. The TD may be a p73 (eg, a human p73) TD or an orthologue or homologue thereof. This may have one or more advantages as follows:-
secretion of tetramers from mammalian or other eukaryotic cells, eg, a mammalian cell disclosed herein such as CHO, HEK293 or Cos;
enhanced yield of secreted tetramers versus monomers;
enhanced yield of secreted tetramers versus dimers;
enhanced yield of secreted tetramers versus trimers;
enhanced yield of secreted tetramers versus monomers and dimers combined;
enhanced yield of secreted tetramers versus monomers, dimers and trimers combined;
enhanced affinity or avidity of antigen binding in tetramers comprising antigen binding sites;
enhanced tetramer production and/or expression, wherein the tetramer is no more than 200 or no more than 160 or 150 kDa in size.
In an embodiment, each polypeptide or monomer comprises one or more VH, VL or VH/VL binding sites of an antibody selected from ReoPro™; Abciximab; Rituxan™; Rituximab; Zenapax™; Daclizumab; Simulect™; Basiliximab; Synagis™; Palivizumab; Remicade™; Infliximab; Herceptin™; Trastuzumab; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Gazyva™ and Obinutuzumab. In an alternative, (eg, for treating or preventing a cancer in a human) each polypeptide or monomer comprise one or more VH, VL or VH/VL binding sites of an antibody selected from ipilimumab (or YERVOY™), tremelimumab, nivolumab (or OPDIVO™), pembrolizumab (or KEYTRUDA™), pidilizumab, BMS-936559, durvalumab and atezolizumab. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

In an example, the tetramer comprises 4 copies of the antigen binding site of a first antibody selected from the group consisting of ipilimumab (or YERVOY™), tremelimumab, nivolumab (or OPDIVO™), pembrolizumab (or KEYTRUDA™), pidilizumab, BMS-936559, durvalumab and atezolizumab and optionally 4 copies of the antigen binding site of a second antibody selected from said group, wherein the first and second antibodies are different. For example, the first antibody is ipilimumab (or YERVOY™) and optionally the second antibody is nivolumab (or OPDIVO™) or pembrolizumab (or KEYTRUDA™). This is useful for treating or preventing a cancer in a human.

In an example, the tetramer comprises 4 copies of the antigen binding site of Avastin. In an example, the tetramer comprises 4 copies of the antigen binding site of Humira. In an example, the tetramer comprises 4 copies of the antigen binding site of Erbitux. In an example, the tetramer comprises 4 copies of the antigen binding site of Actemra™. In an example, the tetramer comprises 4 copies of the antigen binding site of sarilumab. In an example, the tetramer comprises 4 copies of the antigen binding site of dupilumab. In an example, the tetramer comprises 4 copies of the antigen binding site of alirocumab or evolocumab. In an example, the tetramer comprises 4 copies of the antigen binding site of In an example, the tetramer comprises 4 copies of the antigen binding site of Remicade. In an example, the tetramer comprises 4 copies of the antigen binding site of Lucentis. In an example, the tetramer comprises 4 copies of the antigen binding site of Eylea™. Such tetramers are useful for administering to a human to treat or prevent a cancer. Such tetramers are useful for administering to a human to treat or prevent an ocular condition (eg, wet AMD or diabetic retinopathy, eg, when the binding site is an Avastin, Lucentis or Eylea site). Such tetramers are useful for administering to a human to treat or prevent angiogenesis.

In an example, the tetramer comprises 4 copies of insulin. In an example, the tetramer comprises 4 copies of GLP-1. In an example, the tetramer comprises 4 copies of GIP. In an example, the tetramer comprises 4 copies of Exendin-4. In an example, the tetramer comprises 4 copies of insulin and 4 copies of GLP-1. In an example, the tetramer comprises 4 copies of insulin and 4 copies of GIP. In an example, the tetramer comprises 4 copies of insulin and 4 copies of Exendin-4. In an example, the tetramer comprises 4 copies of GLP-1 and 4 copies of Exendin-4. Such tetramers are useful for administering to a human to treat or prevent diabetes (eg, Type II diabetes) or obesity.

### Example Antigens

The polypeptide, multimer may bind to one or more antigens or epitopes, or each of the binding sites herein (eg, dAb or scFv binding sites) herein may bind to an antigen or epitope. In an example, an (or each) antigen herein is selected from the following list. In an example, an (or each) epitope herein is an epitope of an antigen selected from the following list.

Activin type-II receptor; Activin type-IIB receptor; ADAM11; ADAM12; ADAM15; ADAM17; ADAM18; ADAM19; ADAM1A; ADAM1B; ADAM2; ADAM20; ADAM21; ADAM22; ADAM23; ADAM24P; ADAM28; ADAM29; ADAM30; ADAM32; ADAM33; ADAM3A; ADAM3B; ADAM5; ADAM6; ADAM7; ADAM8; ADAM9; ADORA2A; AKT; ALK; alpa-4 integrin; alpha synuclein; anthrax protective antigen; BACE1; BCMA; beta amyloid; BRAF; BTLA; BTNL2; CCR4; CCR5; CD126; CD151; CD16; CD160; CD19; CD20; CD22; CD226; CD244; CD27; CD274 (PDL1); CD276; CD28; CD3; CD30; CD300A; CD300C; CD300E; CD300LB; CD300LF; CD33; CD38; CD3; CD3 epsilon; CD3 delta; CD3 gamma; CD40; CD40L; CD47; CD48; CD5; CD52; CD59; CD6; CD70; CD72; CD73; CD80; CD81; CD84; CD86; CD96; CDK4/6; CEA; CEACAM1; CEACAM3; CGRP receptor; CLEC12A; CLEC1B; CLEC4A; CLEC5A; CLEC7A; Clostridium difficile toxin ; cMET; Complement C5 factor; Complement factor D; CSF1R; CSF2RA; CTAG1B; CTLA4; CXCL12; CXCR2; CXCR4; DR4; DR5; EDA; EDA2R; EGFR; EGFRvIII; EMR1; ENTPD1; EpCAM; Factor IX; Factor X; Factor VII; FAP; FAS; FCAR; FCER1G; FCER2 ; TFR2; 4-1BB; FCGR2A; FCGR2B; FCGR3A; FCGR3B; FCRL1; FCRL3; FCRL4; FCRL6; FGRF1/2/3; FLT3; GAL; GEM; GITR; GITRL; GM-CSF; GM-CSF receptor; GP IIb IIIa; gpNMB; TIM3; HDAC1; HER-2; HER3; HFE; HHLA2; Histone H1 modulator; HLA-C; HLA-G; HMGB1; HMMR; HVEM; ICAM-1; ICOS; ICOSL; IDO1; IFNG; IL-1 beta; IL-12; IL-13; IL-2; IL-22R; IL-23; IL-23a; IL-24; IL-2R; IL-34; IL-8; IL10; IL11; IL13; IL17A; IL17D; IL22; IL2RA; IL36G; IL4; IL4a; IL5; IL6; Immunoglobulin E; Immunoglobulin E; Immunoglobulin G; INHBA; INHBB; Interferon type I receptor; INF-a-2a/2b; INF-b-1a/1b; ITGA2B; ITGB3; KIR; KIR2DL1; KIR2DL2; KIR2DL3; KIR2DL4; KIR2DL5A; KIR3DL1; KIR3DL3 ;KIR3DS1; KIT; KLRC1; KLRC2; KLRF1; KLRG1 ;KLRK1 ;KRAS; LAG3; LAIR1; LAIR2; LFA-1; LIGHT; LILRA1; LILRA2; LILRA3; LILRA4; LILRA5; LILRA6; LILRB1; LILRB2; LILRB3; LILRB4; LILRB5 ;LILRP1; LILRP2; LTA; LTBR; LY9; MadCam; MAGE-C1; MAGE-C2; MARCO; MEK-½; MIA3; MIC; MICA; MICB; MMP9; MS4A1; MS4A2; mTOR; MUC1; MUCIN-1; Nav1.7; Nav1.8; NCR1; NCR2; NCR3; NGF; NGFR; NT5E; NY-ESO-1; OX40; OX40L; p53; PARP; PCSK9; PD-1; PDCD1LG2; PDCD6; PDGF receptor alpha; PECAM1; PI3K delta; PILRA; PPP1R1B; PSMA; PTPN6; PVR; PVRL2; PVRL3; RANKL; Respiratory syncytial virus protein; SIGLEC10; SIGLEC12; SIGLEC15; SIGLEC5; SIGLEC6; SIGLEC7; SIGLEC9; SIRPA; SIRPB1; SLAMF1; SLAMF6; SLAMF7; SLAMF8; SNCA; SOD1; STAT3; STING; SURVIVIN; TARM1; Tau; TDP43; TfR1; TGF-b; TGM2; TIGIT; TIM-3; TLR-4; TLR03; TMEM30a; TMIGD2; TNFa; TNFRSF10A; TNFRSF10B; TNFRSF10C; TNFRSF10D; TNFRSF11A; TNFRSF11B; TNFRSF12A; TNFRSF13B; TNFRSF13C; TNFRSF14; TNFRSF17; TNFRSF18; TNFRSF19; TNFRSF21; TNFRSF4; TNFRSF6B; TNFRSF8; TNFRSF9; TNFSF10; TNFSF11; TNFSF12; TNFSF13; TNFSF13B; TNFSF14; TNFSF15; TNFSF18; TNFSF4; TNFSF8; TNFSF9; Transmembrane glycoprotein NMB modulator; TREML1; TREML2; TSLP; VEGF; VEGF-2R; VEGF1; VEGFA; VEGFL; VEGFR; Viral envelope glycoprotein; Viral protein haemagglutinin; VISTA; VSIG4; VSTM1; VTCN1; and WEE-1. In an example, an antigen herein is a PCSK9, eg, human PCSK9; optionally the multimer has 4, 8, 12 or 16 copies an anti- PCSK9 binding site (eg, a dAbs).

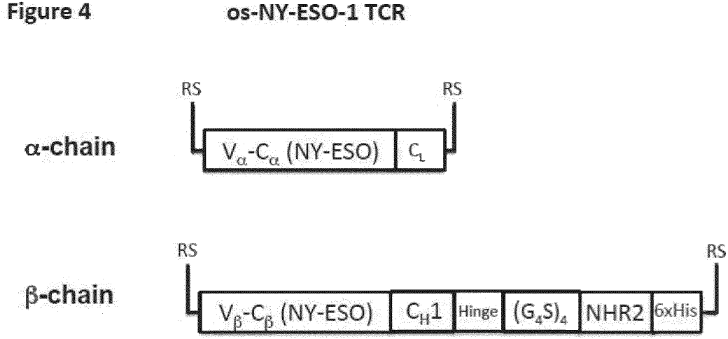

An example antigen is a toxin, such as a snake venom toxin, eg, wherein a multimer of the invention is administered (such as systemically or by IV injection) to a human or animal subject and the antigen binding sites comprised by the multimer specifically bind to the toxin in the subject. Preferably, each binding site or domain of the multimer is a dAb (eg, a Nanobody™). For example, each snake venom toxin antigen binding site of the multimer of the invention is a C33 single domain VH as disclosed in FIG. 4 of PLoS One. 2013 Jul 22;8(7):e69495. doi: 10.1371/journal.pone.0069495; “In vivo neutralization of α-cobratoxin with high-affinity llama single-domain antibodies (VHHs) and a VHH-Fc antibody”, Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C15 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C7 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C13 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C19 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C34 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C31 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C20 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C2 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C29 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C42 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. For example, each snake venom toxin antigen binding site of the multimer of the invention is a C43 single domain VH as disclosed in FIG. 4 of Richard et al, the amino acid of which as discloed in said FIG. 4 is incorporated herein in its entirety by reference for possible use in the present invention as a binding site or domain or dAb or Nanobody or VHH or VH. An example of a snake venom toxin is 3FTx, dendrotoxin or PLA2 toxin. Optionally, the toxin is an alpha-neurotoxin, eg, from Cobra.

Another example of a toxin is a blood toxin, eg, wherein a multimer of the invention is administered (such as systemically or by IV injection) to a human or animal subject and the antigen binding sites comprised by the multimer specifically bind to the toxin in the blood of the subject. These examples are useful for sequestering the toxin or for reducing the toxic effect of the toxin to the subject or to promote excretion or metabolism of the toxin.

In an example, the antigen is a viral antigen, each a capsid protein or carbohydrate (eg, a sugar). In an example, a multimer of the invention binds to a virus or virus antigen, eg, a virus selected from Table 19 wherein the virus comprises a surface antigen that is bound by the multimer; or the multimer of the invention binds to a cell or virus antigen, eg, selected from an antigen disclosed in Table 20. Binding to the virus may, for example, reduce or inhibit attachment of the virus to its host cell or infection of the cell by the virus. For example, the invention provides a method of treating or preventing (eg, reducing the risk of) a viral or cell infection in a human or animal or plant subject (eg, in a human subject), the method comprising administering a multimer of the invention to the subject wherein the multimer binds to a surface antigen of the virus, thereby inhibiting the virus from attaching to a host cell; inhibiting infection of a host cell by the virus and/or sequestering the virus in the subject (eg, to mark the bound virus for clearance from the systemic circulation or a tissue of the subject). In an alternative, For example, the invention provides a method of treating or preventing (eg, reducing the risk of) a bacterial or archaeal cell infection in a human or animal or plant subject (eg, in a human subject), the method comprising administering a multimer of the invention to the subject wherein the multimer binds to a surface antigen of the cell, thereby inhibiting infection of the subject by the cell and/or sequestering the cell in the subject (eg, to mark the bound cell for clearance from the systemic circulation or a tissue of the subject). In an alternative, For example, the invention provides a method of treating or preventing (eg, reducing the risk of) a cancer in a human or animal subject (eg, in a human subject), the method comprising administering a multimer of the invention to the subject wherein the multimer binds to a surface antigen of a tumour cell, thereby sequestering the cell in the subject (eg, to mark the bound cell for clearance from the systemic circulation or a tissue of the subject) or marking the cell for targeting by the immune sytem or another therapy (eg, immune checkpoint therapy or CAR-T therapy) administered to the subject.

In an example, the antigen is selected from CXCR2, CXCR4, GM-CSF, ICAM-1, IFN-g, IL-1, IL-10, IL-12, IL-1R1, IL-1R2, IL-1Ra, IL-1β, IL-4, IL-6, IL-8, MIF, TGF-β, TNF-α, TNFR1, TNFR2 and VCAM-1. Targeting one or more of these antigens may be useful for treating or preventing sepsis in a subject. Thus, in an example the multimer of the invention comprises one or more antigen binding sites (eg, each one provided by a dAb), wherein the multimer is for use in a method of treating or preventing sepsis in a human or animal subject, wherein the multimer is administered to the subject (eg, systemically or intravenously). Optionally, the multimer is monospecific, bispecific, trispecific or tetraspecific for antigen binding. For example, the multimer is bispecific, trispecific or tetraspecific for an antigen selected from CXCR2, CXCR4, GM-CSF, ICAM-1, IFN-g, IL-1, IL-10, IL-12, IL-1R1, IL-1R2, IL-1Ra, IL-1β, IL-4, IL-6, IL-8, MIF, TGF-β, TNF-α, TNFR1, TNFR2 and VCAM-1. There is also provided a pharmaceutical composition comprising such a multimer and a pharmaceutically acceptable diluent, carrier or excipient. There is also provided a method of treating or preventing sepsis in a human or animal subject, the method comprising administering the multimer to the subject, eg, systemically or intravenously.

### Diseases and Conditions

The polypeptide monomer or multimer (eg, dimer, trimer, tetramer or octamer) of the invention can be used in a method for administration to a human or animal subject to treat or prevent a disease or condition in the subject.

Optionally, the disease or condition is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(1) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn’s disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren’s syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy; (aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

### Neurodegenerative or Cns Diseases or Conditions for Treatment Or Prevention

In an example, the neurodegenerative or CNS disease or condition is selected from the group consisting of Alzheimer disease, geriopsychosis, Down syndrome, Parkinson’s disease, Creutzfeldt-jakob disease, diabetic neuropathy, Parkinson syndrome, Huntington’s disease, Machado-Joseph disease, amyotrophic lateral sclerosis, diabetic neuropathy, and Creutzfeldt Creutzfeldt- Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer’s disease) is treated, prevented or progression thereof is reduced. In an embodiment the method causes an increase of IFN-gamma in the CNS system (eg, in the brain and/or CSF) of the subject. In an example, the method restores nerve fibre and//or reduces the progression of nerve fibre damage. In an example, the method restores nerve myelin and//or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatement and/or prevention of CNS and neurodegenerative diseases and conditions, eg, agents such as immune checkpoint inhibitors, eg, anti-PD-1, anti-PD-L1, anti-TIM3 or other antibodies disclosed therein).

### Cancers for Treatment or Prevention

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin’s disease, non-Hodgkin’s lymphoma (indolent and high grade forms), multiple myeloma, Waldenstrom’s macroglobulinemia, heavy chain disease, myelodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing’s tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms’ tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

### Autoimmune Diseases for Treatment or Prevention

Acute Disseminated Encephalomyelitis (ADEM)
Acute necrotizing hemorrhagic leukoencephalitis
Addison’s disease
Agammaglobulinemia
Alopecia areata
Amyloidosis
Ankylosing spondylitis
Anti-GBM/Anti-TBM nephritis
Antiphospholipid syndrome (APS)
Autoimmune angioedema
Autoimmune aplastic anemia
Autoimmune dysautonomia
Autoimmune hepatitis
Autoimmune hyperlipidemia
Autoimmune immunodeficiency
Autoimmune inner ear disease (AIED)
Autoimmune myocarditis
Autoimmune oophoritis
Autoimmune pancreatitis
Autoimmune retinopathy
Autoimmune thrombocytopenic purpura (ATP)
Autoimmune thyroid disease
Autoimmune urticaria
Axonal & neuronal neuropathies
Balo disease
Behcet’s disease
Bullous pemphigoid
Cardiomyopathy
Castleman disease
Celiac disease
Chagas disease
Chronic fatigue syndrome
Chronic inflammatory demyelinating polyneuropathy (CIDP)
Chronic recurrent multifocal ostomyelitis (CRMO)
Churg-Strauss syndrome
Cicatricial pemphigoid/benign mucosal pemphigoid
Crohn’s disease
Cogans syndrome
Cold agglutinin disease
Congenital heart block
Coxsackie myocarditis
CREST disease
Essential mixed cryoglobulinemia
Demyelinating neuropathies
Dermatitis herpetiformis
Dermatomyositis
Devic’s disease (neuromyelitis optica)
Discoid lupus
Dressler’s syndrome
Endometriosis
Eosinophilic esophagitis
Eosinophilic fasciitis
Erythema nodosum
Experimental allergic encephalomyelitis
Evans syndrome
Fibromyalgia
Fibrosing alveolitis
Giant cell arteritis (temporal arteritis)
Giant cell myocarditis
Glomerulonephritis
Goodpasture’s syndrome
Granulomatosis with Polyangiitis (GPA) (formerly called Wegener’s Granulomatosis)
Graves’ disease
Guillain-Barre syndrome
Hashimoto’s encephalitis
Hashimoto’s thyroiditis
Hemolytic anemia
Henoch-Schonlein purpura
Herpes gestationis
Hypogammaglobulinemia
Idiopathic thrombocytopenic purpura (ITP)
IgA nephropathy
IgG4-related sclerosing disease
Immunoregulatory lipoproteins
Inclusion body myositis
Interstitial cystitis
Juvenile arthritis
Juvenile diabetes (Type 1 diabetes)
Juvenile myositis
Kawasaki syndrome
Lambert-Eaton syndrome
Leukocytoclastic vasculitis
Lichen planus
Lichen sclerosus
Ligneous conjunctivitis
Linear IgA disease (LAD)
Lupus (SLE)
Lyme disease, chronic
Meniere’s disease
Microscopic polyangiitis
Mixed connective tissue disease (MCTD)
Mooren’s ulcer
Mucha-Habermann disease
Multiple sclerosis
Myasthenia gravis
Myositis
Narcolepsy
Neuromyelitis optica (Devic’s)
Neutropenia
Ocular cicatricial pemphigoid
Optic neuritis
Palindromic rheumatism
PANDAS (Pediatric Autoimmune Neuropsychiatric Disorders Associated with Streptococcus)
Paraneoplastic cerebellar degeneration
Paroxysmal nocturnal hemoglobinuria (PNH)
Parry Romberg syndrome
Parsonnage-Turner syndrome
Pars planitis (peripheral uveitis)
Pemphigus
Peripheral neuropathy
Perivenous encephalomyelitis
Pernicious anemia
POEMS syndrome
Polyarteritis nodosa
Type I, II, & III autoimmune polyglandular syndromes
Polymyalgia rheumatica
Polymyositis
Postmyocardial infarction syndrome
Postpericardiotomy syndrome
Progesterone dermatitis
Primary biliary cirrhosis
Primary sclerosing cholangitis
Psoriasis
Psoriatic arthritis
Idiopathic pulmonary fibrosis
Pyoderma gangrenosum
Pure red cell aplasia
Raynauds phenomenon
Reactive Arthritis
Reflex sympathetic dystrophy
Reiter’s syndrome
Relapsing polychondritis
Restless legs syndrome
Retroperitoneal fibrosis
Rheumatic fever
Rheumatoid arthritis
Sarcoidosis
Schmidt syndrome
Scleritis
Scleroderma
Sjogren’s syndrome
Sperm & testicular autoimmunity
Stiff person syndrome
Subacute bacterial endocarditis (SBE)
Susac’s syndrome
Sympathetic ophthalmia
Takayasu’s arteritis
Temporal arteritis/Giant cell arteritis
Thrombocytopenic purpura (TTP)
Tolosa-Hunt syndrome
Transverse myelitis
Type 1 diabetes
Ulcerative colitis
Undifferentiated connective tissue disease (UCTD)
Uveitis
Vasculitis
Vesiculobullous dermatosis
Vitiligo
Wegener’s granulomatosis (now termed Granulomatosis with Polyangiitis (GPA).

### Inflammatory Diseases for Treatment or Prevention

Alzheimer’s
ankylosing spondylitis
arthritis (osteoarthritis, rheumatoid arthritis (RA), psoriatic arthritis)
asthma
atherosclerosis
Crohn’s disease
colitis
dermatitis
diverticulitis
fibromyalgia
hepatitis
irritable bowel syndrome (IBS)
systemic lupus erythematous (SLE)
nephritis
Parkinson’s disease
ulcerative colitis.

### Multivalent Soluble TCR

The present configuration relates to a multivalent soluble TCR protein. In one aspect, the invention relates to tetravalent and octavalent soluble TCR analogues. The TCR proteins of the invention are capable of self-assembly from monomers and are entirely of human origin. The proteins are multimers which comprise an ETO NHR2 multimerisation domain. The invention also relates to methods of constructing multimeric soluble TCRs, and methods of using such proteins.

Attempts to exploit alternative soluble TCR formats as therapeutic molecules have lagged far behind compared to the plethora of antibody formats. This is largely due to TCR, a heterodimeric transmembrane protein having the intrinsic problem of solubility once the extracellular TCR α/β chains are dissociated from their transmembrane and cytoplasmic domain. Secondly the intrinsic low affinity and avidity of these molecules for their cognate ligand at the target site has to a large degree hampered their development as a therapeutic molecule.

In order to overcome these drawbacks, the present configuration of the invention provides a TCR protein which is both multivalent and soluble. Multivalency increases the avidity of the TCR for cognate pMHC, and solubility allows the TCR to be used outside of a transmembrane environment. Accordingly, in a first aspect there is provided a multivalent heterodimeric soluble T cell receptor capable of binding pMHC complex comprising:
(i) TCR extracellular domains;
(ii) (ii) immunoglobulin constant domains; and
(iii) (iii) an NHR2 multimerisation domain of ETO.

The use of Ig constant domains provides the TCR extracellular domains with stability and solubility; multimerisation via the NHR2 domains provides multivalency and increased avidity. Advantageously, all of the domains are of human origin or conform to human protein sequences.

Using the Ig constant domain to stabilise and render soluble the TCR avoids the use of non-native disulphide bonds. Advantageously, therefore, the TCR of the invention does not comprise a non-native disulphide bond.

In one embodiment, said complex comprises a heavy chain and a light chain, and each light chain comprises a TCR Vα domain and an immunoglobulin C_{α} domain, and each heavy chain comprises a TCR Vβ domain and an immunoglobulin C_{H}1 domain.

In one embodiment, each light chain additionally comprises a TCR Cα domain, and each heavy chain additionally comprises a TCR Cβ domain.

In embodiments, the TCR and immunoglobulin domains can be separated by a flexible linker.

The NHR2 multimerisation domain is advantageously attached to the C-terminus of an immunoglobulin domain. Thus, each dimer of heavy and light chains will be attached to one multimerisation domain, so that the heavy chain-light chain dimers associate into multivalent oligomers.

In embodiments, the multimerisation domain and the immunoglobulin domain are separated by a flexible linker. In certain embodiments, this allows the multimerisation domain to multimerise without hindrance from the immunoglobulin domain(s).

In embodiments, the TCR protein may further comprise an immunoglobulin hinge domain. Hinge domains allow dimerization of heavy chain-light chain dimers; this allows further multimerisation of the TCR proteins. For example, a multimerisation domain which forms polypeptide tetramers can, using an immunoglobulin hinge domain, form multimers up to polypeptide octamers. Likewise, a dimerising multimerisation domain can form tetramers in the presence of a hinge domain.

In embodiments, the TCR protein of the invention is tetravalent.

In embodiments, the TCR protein of the invention is octavalent

The present invention provides a soluble TCR where it is stably assembled in a tetravalent heterodimeric format using the nervy homology region 2 (NHR2) domain found in the ETO family protein in humans (Liu et al. 2006). The NHR2 domain is found naturally to form homotetramer, which is formed from pairing of two NHR2 homodimers. NHR2 linked operably to the extracellular TCRα or TCRβ chain will preferentially form tetravalent heterodimeric soluble TCR protein molecules sequentially self-assembled from a monomer followed by a homodimer (FIG. 1).

TCR proteins assembling into octamers can be created using the NHR2 domain, by employing immunoglobulin hinge domains.

In a further aspect, the TCR proteins of the invention can be coupled to biologically active polypeptides/effector molecules. Examples of such polypeptides can include immunologically active moieties such as cytokines, binding proteins such as antibodies or targeted polypeptides, and the like.

The invention further relates to methods for making tetravalent and octavalent heterodimeric soluble TCR, the DNA vectors encoding the proteins used for transfecting host cells of interests and the use of these novel highly sensitive multivalent soluble TCR protein molecules. Applications for use could include but not limited to, therapeutics, diagnostics and drug discovery.

In a further aspect, the invention provides a method for constructing multivalent immunoglobulin molecules in an efficient manner, without employing non-human construct components.

Accordingly, there is provided a multimeric immunoglobulin comprising
(i) immunoglobulin variable domains; and
(ii) an NHR2 multimerisation domain of ETO.

The immunoglobulin variable domains are preferably antibody variable domains. Such domains are fused to the ETO NHR2 multimerisation domain, which provides means for forming tetramers of the immunoglobulin variable domains.

The ETO NHR2 domain is more efficient than p53 and similar multimerisation domains in the production of immunoglobulin multimers, and permits the production of multimeric immunoglobulin molecules without the use of non-human components in the construct.

Also provided is a method for producing a multimeric immunoglobulin, comprising expressing immunoglobulin variable domains in fusion with an NHR2 domain of ETO, and allowing the variable domains to assemble into multimers.

Preferably, the immunoglobulin variable domains are attached to one or more immunoglobulin constant domains.

Advantageously, the immunoglobulin domains are antibody domains. For example, the variable domains can be V_{H} and V_{L} antibody domains. For example, the constant domains are antibody CH1 domains.

In one embodiment, the multimeric immunoglobulin molecules according to the invention, both TCR and non-TCR immunoglobulins, are produced for screening by phage display or another display technology. For example, therefore, the multivalent immunoglobulins are produced as fusions with a phage coat protein. For each immunoglobulin produced fused to a coat protein, other immunoglobulin molecules are produced without a coat protein, such that they can assemble on the phage surface as a result of NHR2 multimerisation.

The present configuration of the invention as detailed above relates to the nucleic acid sequences and methods for producing novel multivalent, for example tetravalent and octavalent, soluble proteins. In one aspect in particular the soluble protein is a TCR assembled into a tetravalent heterodimeric format that can bind four pMHC with high sensitivity, affinity and specificity. The soluble tetravalent heterodimeric TCR is a unique protein molecule composed from either the entire or in part the extracellular TCR α/β chains. The extracellular TCR α/β chains are linked to immunoglobulin C_{H}1 and C_{L} (either C_{K} or Cλ) domains. This linkage allows stable formation of heterodimeric TCR α/β. In the context of soluble tetravalent TCR the unique feature is the NHR2 homotetramer domain of the ETO family of proteins, which is operably linked to the C-terminus of C_{H}1 or the C-terminus of C_{L}. Linkage of the NHR2 domain to the heterodimeric α/βTCR in this manner allows it to self-assemble into a tetravalent format inside cells and be subsequently secreted into the supernatant as a soluble protein.

### TCR Extracellular Domains

TCR extracellular domains are composed of variable and constant regions. These domains are present in T-cell receptors in the same way as they are present in antibodies and other immunoglobulin domains. The TCR repertoire has extensive diversity created by the same gene rearrangement mechanisms used in antibody heavy and light chain genes (Tonegawa, S. (1988) Biosci. Rep. 8:3-26). Most of the diversity is generated at the junctions of variable (V) and joining (J) (or diversity, D) regions that encode the complementarity determining region 3 (CDR3) of the α and β chains (Davis and Bjorkman (1988) Nature 334:395-402). Databases of TCR genes are available, such as the IMGT LIGM database, and methods for cloning TCRs are known in the art - for example, see Bentley and Mariuzza (1996) Ann. Rev. Immunol. 14:563-590; Moysey et al., Anal Biochem. 2004 Mar 15;326(2):284-6; Walchli, et al. (2011) A Practical Approach to T-Cell Receptor Cloning and Expression. PLoS ONE 6(11): e27930.

### Immunoglobulin Variable Domains

Antibody variable domains are known in the art and available from a wide variety of sources. Databases of sequences of antibody variable domains exist, such as IMGT and Kabat, and variable domains can be produced by cloning and expression of natural sequences, or synthesis of artificial nucleic acids according to established techniques.

Methods for the construction of bacteriophage antibody display libraries and lambda phage expression libraries are well known in the art (McCafferty et al. (1990) Nature, 348: 552; Kang et al. (1991) Proc. Natl. Acad. Sci. USA., 88: 4363; Clackson et al. (1991) Nature, 352: 624; Lowman et al. (1991) Biochemistry, 30: 10832; Burton et al. (1991) Proc. Natl. Acad Sci USA., 88: 10134; Hoogenboom et al. (1991) Nucleic Acids Res., 19: 4133; Chang et al. (1991) J Immunol., 147: 3610; Breitling et al. (1991) Gene, 104: 147; Marks et al. (1991) supra; Barbas et al. (1992) supra; Hawkins and Winter (1992) J Immunol., 22: 867; Marks et al., 1992, J Bioi. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) Nature, 352: 624; Marks et al. (1991) J Mol. Bioi., 222: 581; Chiswell et al. (1992) Trends Biotech., 10: 80; Marks et al. (1992) J Bioi. Chem., 267). Various embodiments of scFv libraries displayed on bacteriophage coat proteins have been described. Refinements of phage display approaches are also known, for example as described in W096/06213 and W092/01047 (Medical Research Council et al.) and W097/08320 (Morphosys), which are incorporated herein by reference.

Such techniques can be adapted for the production of multimeric immunoglobulins by the fusion of NHR2 multimerisation domains to the antibody variable domains

### Immunoglobulin Constant Domains

An immunoglobulin constant domain, as referred to herein, is preferably an antibody constant domain. Constant domains do vary in sequence between antibody subtypes; preferably, the constant domains are IgG constant domains. Preferably, the constant domains are CH1 constant domains. Antibody constant domains are well known in the art and available from a number of sources and databases, including the IMGT and Kabat databases.

The fusion of antibody constant domains to immunoglobulin variable domains is also known in the art, for example in the construction of engineered Fab antibody fragments.

### Linkers

Flexible linkers can be used to connect TCR variable domain - Ig constant domain to the NHR2 multimerisation domain. This allows the TCR domains and the multimerisation domain to function without steric hindrance from each other or other molecules in the multimeric complex. Suitable linkers comprise, for example, glycine repeats, glycine-alanine repeats, Gly(4)Ser linkers, or flexible polypeptide linkers as set forth in Reddy Chichili et al., 2012 Protein Science 22:153-167.

### Immunoglobulin Hinge Domain

The Ig Hinge domain, herein preferably an antibody hinge domain, is the domain which links antibody constant regions in a natural antibody. This domain therefore provides for natural dimerization of molecules which include an antibody constant domain. It is present, for example, in a F(ab)2 antibody fragment, as well as whole antibodies such as IgG. This region comprises two natural interchain disulphide bonds, which connect the two CH1 constant domains together.

The multimerisation domain, in one embodiment, may be attached to the Ig constant domain or to the hinge domain. If a hinge domain is present, the multimerisation domain will form a TRC octamer, comprising four dimers of TCR variable-Ig Constant domains joined at a hinge region. Without the hinge region, the multimerisation domain will lead to the formation of a tetramer. Preferably, the multimerisation domain is attached to the C-terminal end of the constant domain or the hinge region.

### Biologically Active Molecule

One or more biologically active molecules or effector molecules (EM) can be attached to the multimer, eg, multimeric TCR proteins, of the present invention. Such molecules may be, for example, antibodies, especially antibodies which may assist in immune recognition and functioning of the TCR, such as anti-CD3 antibodies or antibody fragments.

In some aspects, the biologically active molecule can be a cytotoxic drug, toxin or a biologically active molecule such as a cytokine, as described in more detail below. Examples of biologically active molecules include chemokines such as MIP-1b, cytokines such as IL-2, growth factors such as GM-CSF or G-CSF, toxins such as ricin, cytotoxic agents, such as doxorubicin or taxanes, labels including radioactive and fluorescent labels, and the like. For examples of biologically active molecules conjugatable to TCRs, see US20110071919.

In other aspects, the biologically active molecule is, for example, selected from the group consisting of: a group capable of binding to a molecule which extends the half-life of the polypeptide ligand in vivo, and a molecule which extends the half-life of the polypeptide ligand in vivo. Such a molecule can be, for instance, HSA or a cell matrix protein, and the group capable of binding to a molecule which extends the half-life of the TCR molecule in vivo is an antibody or antibody fragment specific for HSA or a cell matrix protein.

In one embodiment, the biologically active molecule is a binding molecule, for example an antibody fragment. 2, 3, 4, 5 or more antibody fragments may be joined together using suitable linkers. The specificities of any two or more of these antibody fragments may be the same or different; if they are the same, a multivalent binding structure will be formed, which has increased avidity for the target compared to univalent antibody fragments.

The biologically active molecule can moreover be an effector group, for example an antibody Fc region.

Attachments to the N or C terminus may be made prior to assembly of the TCR molecule or engineered polypeptide into multimers, or afterwards. Thus, the TCR fusion with an Ig Constant domain may be produced (synthetically, or by expression of nucleic acid) with an N or C terminal biologically active molecule already in place. In certain aspects, however, the addition to the N or C terminus takes place after the TCR fusion has been produced. For example, Fluorenylmethyloxycarbonyl chloride can be used to introduce the Fmoc protective group at the N-terminus of the TCR fusion. Fmoc binds to serum albumins including HSA with high affinity, and Fmoc-Trp or FMOC-Lys bind with an increased affinity. The peptide can be synthesised with the Fmoc protecting group left on, and then coupled with the scaffold through the cysteines. An alternative is the palmitoyl moiety which also binds HSA and has, for example been used in Liraglutide to extend the half-life of this GLP-1 analogue.

Alternatively, the TCR fusinon can be modified at the N-terminus, for example with the amine- and sulfhydryl-reactive linker N-e-maleimidocaproyloxy)succinimide ester (EMCS). Via this linker the TCR can be linked to other polypeptides, for example an antibody Fc fragment.

### The NHR2 Domain

AML1/ETO is the fusion protein resulting from the t(8;21) found in acute myeloid leukemia (AML) of the M2 subtype. AML1/ETO contains the N-terminal 177 amino acids of RUNX1 fused in frame with most (575 aa) of ETO. The nervy homology domain 2 of ETO is responsible for many of the biological activities associated with AML1/ETO, including oligomerisation and protein-protein interactions. This domain is characterised in detail in Liu et al (2006). See Genbank accession number NG_023272.2.

In one aspect of the present invention, the protein assembled into a soluble multivalent format is a TCR composed of either in part or all of the extracellular domains of the TCR α and β chains. The TCR α and β chains are stabilized by immunoglobulin C_{H}1 and C_{L} domains and could be arranged in the following configurations:
1. Vα-C_{L} and VβC_{H}1
2. Vα-C_{H}1 and Vβ-C_{L}
3. VαCα-C_{L} and VβCβ-C_{H}1
4. VαCαC_{H}1 and VβCβC_{L}

In one aspect of this invention, the extracellular TCR domains are linked to immunoglobulin C_{H}1 and C_{L} domains via an optional peptide linker (L) to promote protein flexibility and facilitate optimal protein folding.
1. Vα-(L)-C_{L} and Vβ-(L)-C_{H}1
2. Vα-(L)-C_{H}1 and Vβ-(L)-C_{L}
3. VαCα-(L)-C_{L} and VβCβ-(L)-C_{H}1
4. VαCα-(L)-C_{H}1 and VβCβ-(L)-C_{L}

In another aspect of this invention, a tetramerisation domain (TD) such as NHR2 homotetramer domain is linked to the C-terminus of either the immunoglobulin C_{H}1 or C_{L} domain, which is linked to the extracellular TCR α and β chain. The NHR2 domain could be optionally linked to C_{H}1 or C_{L} domain via a peptide linker. The resulting tetravalent heterodimeric TCR protein could be arranged in the following configurations where (L) is an optional peptide linker:
1. Vα-(L)-C_{L} and Vβ-(L)-C_{H}1-(L)-TD
2. Vα-(L)-C_{H}1 -(L)-TD and Vβ-(L)-C_{L}
3. VαCα-(L)-C_{L} and VβCβ-(L)-C_{H}1-(L)-TD
4. VαCα-(L)-C_{H}1-(L)-TD and VβCβ-(L)-C_{L}
5. Vα-(L)-C_{L}-(L)-TD and Vβ-(L)-C_{H}1
6. Vα-(L)-C_{H}1 and Vβ-(L)-C_{L}-(L)-TD
7. VαCα-(L)-C_{L}-(L)-TD and VβCβ-(L)-C_{H}1
8. VαCα-(L)-C_{H}1and VβCβ-(L)-C_{L}-(L)-TD

The sensitivity of the soluble TCR for its cognate pMHC can be enhanced by increasing the avidity effect. This is achieved by increasing the number of antigen binding sites, facilitated by the tetramerisation domain. This in turn also increases the molecular weight of the protein molecule compared to a monovalent soluble TCR and thus extends serum retention in circulation. Increasing the serum half-life also enhances the likelihood of these molecules interacting with their cognate target antigens.

The tetravalent heterodimeric soluble TCR protein molecule is capable of binding simultaneously to one, two, three or four pMHC displayed on a single cell or bind simultaneously to one, two, three or four different cells displaying its cognate pMHC.

TCR α and β chain sequences used in this invention could be from a known TCR specific for a particular pMHC or identified de novo by screening using techniques known in the art, such as phage display. Furthermore, TCR sequences are not limited to α and β chain in this invention but can also incorporate TCRδ and γ or ε chain and sequence variations thereof either directly cloned from human T cells or identified by directed evolution using recombinant DNA technology.

In another aspect to this invention, the tetravalent heterodimeric soluble TCR protein molecules are preferentially produced in mammalian cells for optimal production of soluble, stable and correctly folded protein molecules.

Multimer (eg, tetramer or octamer), or multivalent TCR according to the present invention may be expressed in cells, such as mammalian cells, using any suitable vector system. The pTT5 expression vector is one example of an expression system is used to express multivalent soluble TCR. The pTT5 expression system allows for high-level transient production of recombinant proteins in suspension-adapted HEK293 EBNA cells (Zhang et al. 2009). It contains origin of replication (oriP) that is recognized by the viral protein Epstein-Barr Nuclear Antigen 1 (EBNA-1), which together with the host cell replication factor mediates episomal replication of the DNA plasmid allowing enhanced expression of recombinant protein. Other suitable vector system for mammalian cell expression known in the art and commercially available can be used with this invention.

The tetravalent heterodimeric soluble TCR protein molecules or other multimers can be produced by transiently expressing genes from an expression vector.

In another embodiment, tetravalent heterodimeric soluble TCR protein molecules or other multimers can be produced from an engineered stable cell line. Cell lines can be engineered to produce the protein molecule using genome-engineering techniques known in the art where the gene(s) encoding for the protein molecule is integrated into the genome of the host cells either as a single copy or multiple copies. The site of DNA integration can be a defined location within the host genome or randomly integrated to yield maximum expression of the desired protein molecule. Genome engineering techniques could include but not limited to, homologous recombination, transposon mediated gene transfer such as PiggyBac transposon system, site specific recombinases including recombinase-mediated cassette exchange, endonuclease mediated gene targeting such as CRISPR/Cas9, TALENs, Zinc-finger nuclease, meganuclease and virus mediated gene transfer such as Lentivirus.

Also, in another aspect to the invention, the tetravalent heterodimeric soluble TCR protein molecule or other multimer is produced by overexpression in the cytoplasm of E. coli as inclusion bodies and refolded in vitro after purification by affinity chromatography to produce functional protein molecules capable of correctly binding to its cognate pMHC or antigen.

In another aspect to the invention, expression of the tetravalent heterodimeric soluble TCR protein molecule or other multimer is not limited to mammalian or bacterial cells but can also be expressed and produced in insect cells, plant cells and lower eukaryotic cells such as yeast cells.

In another aspect to this invention, the heterodimeric soluble TCR molecule or other multimer is produced as an octavalent protein complex, eg, having up to eight binding sites for its cognate pMHC (FIG. 2). The multiple antigen binding sites allow this molecule to bind up to eight pMHC displayed on one cell or bind pMHC displayed on up to eight different cells thus creating a highly sensitive soluble TCR.

The heterodimeric soluble TCR portion of the molecule is made into a bivalent molecule by fusing the immunoglobulin hinge domain to the C-terminus of either the C_{H}1 or C_{L} domain, which is linked itself either to TCR α or β chain. The hinge domain allows for the connection of two heavy chains giving a structure similar to IgG. To the C-terminus of the hinge domain, a tetramerisation domain such as NHR2 is linked via an optional peptide linker. By joining immunoglobulin hinge to C- and N-terminus of Ig CH1 or CL domain and NHR2 domain respectively, it allows for the assembly of two NHR2 monomers referred to as monomer². In this conformation we predict the two NHR2 domains will most likely not form a homodimer by an antiparallel association due to structural constraints unless a long flexible linker is provided between the hinge and NHR2 domain. Linkage of the tetramerisation and the hinge domain to the to the heterodimeric soluble TCR via immunoglobulin C_{H}1 or C_{L} domain allows for the stepwise self-assembly of an octavalent soluble TCR formed through a NHR2 homotetramer². The self-assembly of the octavalent soluble TCR is via NHR2 monomer² and homodimer² intermediate protein complexes (FIG. 2). The resulting octavalent heterodimeric soluble TCR protein molecule will have superior sensitivity for its cognate pMHC thus giving it a distinctive advantage of identifying unknown antigen or pMHC without having to affinity mature the TCR for its pMHC ligand much beyond affinities seen naturally. In particular it would be useful for identifying pMHC recognized by uncharacterized tumour-specific T cells and T cells involved in other diseases such as autoimmune diseases. A number of different configurations of the octavalent heterodimeric soluble TCR protein molecules can be produced. Some examples are shown below.
1. Vα-(L)-C_{L} and Vβ-(L)-C_{H}1-Hinge-(L)-TD
2. Vα-(L)-C_{H}1-Hinge-(L)-TD and Vβ-(L)-C_{L}
3. Vα-Cα-(L)-C_{L} and Vβ-Cβ-(L)-C_{H}1-Hinge-(L)-TD
4. VαCα(L)-C_{H}1-Hinge-(L)-TD and VβCβ-(L)-C_{L}
5. Vα-(L)-C_{L}-(L)-TD and Vβ-(L)-C_{H}1-Hinge
6. Vα-(L)-C_{H}1-Hinge and Vβ-(L)-C_{L}-(L)-TD
7. Vα-Cα-(L)-C_{L}-(L)-TD and Vβ-Cβ(L)-C_{H}1-Hinge
8. Vα-Cα-(L)-C_{H}1-Hinge and Vβ-Cβ-(L)-C_{L}-(L)-TD

In another aspect to this invention, the self-assembled multivalent protein preferentially tetravalent and octavalent heterodimeric soluble TCR are fused or conjugated to biologically active agent/effector molecule thus allowing these molecules to be guided to the desired cell population such as cancers cells and exert their therapeutic effect specifically. The tumour targeting ability of monoclonal antibodies to guide an effector molecule such as a cytotoxic drug, toxins or a biologically active molecule such as cytokines is well established (Perez et al. 2014; Young et al. 2014). In a similar manner the multivalent soluble TCR molecules outlined in this invention can also be fused with effector proteins and polypeptide or conjugated to cytotoxic agents. Examples of effector protein molecules suitable for use as a fusion protein with the multivalent protein complexes outlined in this invention include but are not limited to, IFNα, IFNβ, IFNγ, IL-2, IL-11, IL-13, granulocyte colony-stimulating factor [G-CSF], granulocyte-macrophage colony-stimulating factor [GM-CSF], and tumor necrosis factor [TNF]α, IL-7, IL-10, IL-12, IL-15, IL-21, CD40L, and TRAIL, the costimulatory ligand is B7.1 or B7.2, the chemokines DC-CK1, SDF-1, fractalkine, lyphotactin, IP-10, Mig, MCAF, M1P-1α, MIP-⅓, IL-8, NAP-2, PF-4, and RANTES or an active fragment thereof. Examples of toxic agent suitable for use as a fusion protein or conjugated to the multivalent protein complexes described in this invention include but not limited to, toxins such as diphtheria toxin, ricin, Pseudomonas exotoxin, cytotoxic drugs such as auristatin, maytansines, calicheamicin, anthracyclines, duocarmycins, pyrrolobenzodiazepines. The cytotoxic drug can be conjugated by a select linker, which is either non-cleavable or cleavable by protease or is acid-labile.

To eliminate heterogeneity and improve conjugate stability the cytotoxic drug can be conjugated in a site-specific manner. By engineering specific cysteine residues or using enzymatic conjugation through glycotransferases and transglutaminases can achieve this (Panowski et al. 2014).

In another aspect of the invention, the multivalent protein complex is covalently linked to molecules allowing detection, such as fluorescent, radioactive or electron transfer agents.

In another aspect of the invention, an effector molecule (EM) is fused to the multivalent protein complex via the C-terminus of the tetramerisation domain such as NHR2 via an optional peptide linker. Fusion via the NHR2 domain can be arranged to produce multivalent protein complexes in a number of different configurations. Examples of some of the protein configurations that can be produced using the tetravalent heterodimeric soluble TCR is shown below:
1. Vα-(L)-C_{L} and Vβ-(L)-C_{H}1-(L)-TD-(L)-EM
2. Vα-(L)-C_{H}1-(L)-TD-(L)-EM and Vβ-(L)-C_{L}
3. Vα-Cα-(L)-C_{L} and Vβ-Cβ-(L)-C_{H}1-(L)-TD-(L)-EM
4. Vα-Cα-(L)-C_{H}1-(L)-TD-(L)-EM and Vβ-Cβ-(L)-C_{L}
5. Vα- (L)-C_{L}-(L)-TD-(L)-EM and Vβ-(L)-C_{H}1
6. Vα-(L)-C_{H}1 and Vβ-(L)-C_{L}-(L)-TD-(L)-EM
7. Vα-Cα-(L)-C_{L}-(L)-TD-(L)-EM and Vβ-Cβ-(L)-C_{H}1
8. Vα-Cα-(L)-C_{H}1 and Vβ-Cβ-(L)-C_{L}-(L)-TD-(L)-EM

In another aspect of the invention, the effector molecule (EM) is fused to the multivalent protein complex at the C-terminus of either the immunoglobulin CH1 or CL1 domain via an optional peptide linker. Fusion of the EM via the immunoglobulin domain can be arranged to produce multivalent protein complexes in a number of different configurations. Examples of some of the protein configurations that can be produced using the tetravalent heterodimeric soluble TCR is shown below:
9. Vα-(L)-C_{L}-(L)-EM and Vβ-(L)-C_{H}1-(L)-TD
10. Vα-(L)-C_{H}1-(L)-TD and Vβ-(L)-C_{L}-(L)-EM
11. Vα-Cα-(L)-C_{L}-(L)-EM and Vαβ-Cβ-(L)-C_{H}1-(L)-TD
12. Vα-Cα-(L)-C_{H}1-(L)-TD and Vβ-Cβ-(L)-C_{L}-(L)-EM
13. Vα-(L)-C_{L}-(L)-TD and Vβ-(L)-C_{H}1-(L)-EM
14. Vα-(L)-C_{H}1-(L)-EM and Vβ-(L)-C_{L}-(L)-TD
15. Vα-Cα-(L)-C_{L}-(L)-TD and Vβ-Cβ-(L)-C_{H}1-(L)-EM
16. Vα-Cα-(L)-C_{H}1-(L)-EM and Vβ-Cβ-(L)-C_{L}-(L)-TD

In another aspect of the invention, effector molecules (EM) are fused to the multivalent protein complex at the C-terminus of either the immunoglobulin CH1 or CL1 domain and also the C-terminus of the tetramerisation domain (e.g. NHR2) via an optional peptide linkers. This approach allows for the fusion of two effector molecules to be fused per TCR heterodimer complex. Fusion of the EM via the immunoglobulin domain and the tetramerisation domain can be arranged to produce multivalent protein complexes in a number of different configurations. Examples of some of the protein configurations that can be produced using the tetravalent heterodimeric soluble TCR is shown below:
17. Vα-(L)-C_{L}-(L)-EM and Vβ-(L)-C_{H}1-(L)-TD-(L)-EM
18. Vα-(L)-C_{H}1-(L)-TD-(L)-EM and Vβ-(L)-C_{L}-(L)-EM
19. Vα-Cα-(L)-C_{L}-(L)-EM and VβCβ-(L)-C_{H}1-(L)-TD-(L)-EM
20. Vα-Cα-(L)-C_{H}1-(L)-TD-(L)-EM and Vβ-Cβ-(L)-C_{L}-(L)-EM
21. Vα-(L)-C_{L}-(L)-TD-(L)-EM and Vβ-(L)-C_{H}1-(L)-EM
22. Vα-(L)-C_{H}1-(L)-EM and Vβ-(L)-C_{L}-(L-)TD-(L)-EM
23. Vα-Cα-(L)-C_{L}-(L)-TD-(L)-EM and Vβ-Cβ-(L)-C_{H}1-(L)-EM
24. Vα-(L)-C_{H}1-(L)-EM and Vβ-Cβ-(L)-C_{L}-(L)-TD-(L)-EM

In another aspect of the invention, the multivalent protein complex is fused to a protein tag to facilitate purification. Purification tags are known in the art and they include, without being limited to, the following tags: His, GST, TEV, MBP, Strep, FLAG.

### Non-TCR Multimers

The present invention provides a unique method for assembling proteins in a soluble multivalent format with potential to bind multiple interacting domains or antigens. The protein can be a monomer, homodimer, heterodimer or oligomer preferentially involved either directly or indirectly in the immune system, or having the potential to regulate immune responses. Examples include, but not limited to, TCR, peptide MHC class I and class II, antibodies or antigen-binding portions thereof and binding proteins having alternative non-antibody protein scaffolds.

In another aspect of the invention, the interacting domains or antigens could be any cell surface expressed or secreted proteins, peptide-associated with MHC Class I or II or any proteins associated with pathogens including viral and bacterial proteins.

Non-TCR multimers may be multimers of antibodies or antibody fragments, such as dAbs of Fabs. Examples of dAbs and Fabs in accordance with the invention include the following:

Examples of multivalent dAbs
25. VH-(L)-NHR2
26. VL(λ or κ)-(L)-NHR2
27. VH-(L)-NHR2-(L)-EM
28. VL(λ or κ)-(L)-NHR2-(L)-EM
29. VH-CH1-(L)-NHR2
30. VL(λ or κ)-CL-(L)-NHR2
31. VH-CH1-(L)-NHR2-(L)-EM
32. VL(λ or κ)-CL-(L)-NHR2-(L)-EM

Examples of multivalent Fabs
33. VH-CH1-(L)-NHR2 and VL(λ or κ)-CL
34. VL(λ or κ)-CL-(L)-NHR2 and VH-CH1
35. VH-CH1-Hinge-(L)-NHR2 and VL(λ or κ)-CL
36. VL(λ or κ)-CL-Hinge-(L)-NHR2 and VH-CH1
37. VH-CH1-(L)-NHR2-(L)-EM and VL(λ or κ)-CL
38. VL(λ or κ)-CL-(L)-NHR2-(L)-EM and VH-CH1
39. VH-CH1-Hinge-(L)-NHR2-(L)-EM and VL(λ or κ)-CL
40. VL(λ or κ)-CL-Hinge-(L)-NHR2-(L)-EM and VH-CH1
41. VH-CH1-(L)-NHR2 and VL(λ or κ)-CL-(L)-EM
42. VL(λ or κ)-CL-(L)-NHR2 and VH-CH1-(L)-EM
43. VH-CH1-Hinge-(L)-NHR2 and VL(λ or κ)-CL-(L)-EM 44. VL(λ or κ)-CL-Hinge-(L)-NHR2 and VH-CH1-(L)-EM

In the examples above, (L) denotes an optional peptide linker, whilst EM denotes a biologically active agent or effector molecule such as toxins, drugs or cytokines, and including binding molecules such as antibodies, Fabs and ScFv.

The variable light chain can be either Vλ or Vκ.

In one aspect of the invention, the assembled tetramerized protein molecule in one example could be a human pMHC for the application in drug discovery using animal drug discovery platforms (e.g. mice, rats, rabbits, chicken). In such a context, the tetramerisation domain is preferentially expressed and produced from genes originating from the animal species it is intended for. One example of such drug discovery applications would be the use of the tetramerized human pMHC as an antigen for immunization in rats for example. Once rats are immunized with pMHC the immune response is directed specifically towards the human pMHC and not the tetramerisation domain of the protein complex.

Multivalent antibodies can be produced, for example using single domain antibody sequences, fused to the NHR2 multimerisation domain.

In a related aspect to the invention, the tetravalent protein can be a peptide used as a probe for molecular imaging of tumour antigens. The multivalent binding of such a probe will have distinctive advantage over monovalent molecular probes as it will have enhanced affinity, avidity and retention time in vivo and this in turn will enhance in vivo tumour targeting.

The multimerisation domain is the NHR2 domain set forth above. Preferably, polypeptides are stabilized and/or rendered soluble by the use of Ig constant domains fused to the polypeptides, such that the fusions provide tetramers of polypeptides. Ig hinge domains can be used to provide octamers.

### Uses of Multimers

Multimeric TCR proteins according to the invention are useful in any application in which soluble TCR proteins are indicated. Particular advantages of the TCR proteins of the invention include increased avidity for the selected target, and/or the ability to bind a plurality of targets.

Thus, in one aspect, the multivalent heterodimeric soluble TCR protein molecules of the invention can be used for selectively inhibiting immune responses, for example suppression of an autoimmune response. The multivalent, for example tetravalent, nature of these soluble protein molecules gives it exquisite sensitivity and binding affinity to compete antigen-specific interactions between T cells and antigen presenting cells. This kind of neutralization effect can be therapeutically beneficial in autoimmune diseases such as rheumatoid arthritis, systemic lupus erythematosus, psoriasis, inflammatory bowel diseases, graves disease, vasculitis and type 1 diabetes.

Similarly, the tetravalent heterodimeric soluble TCR protein molecules can be used to prevent tissue transplant rejection by selectively suppressing T cell recognition of specific transplantation antigen and self antigens binding to target molecule and thus inhibiting cell-to-cell interaction.

In another aspect of the invention, the tetravalent heterodimeric soluble TCR protein molecules can be used in clinical studies such as toxicity, infectious disease studies, neurological studies, behavior and cognition studies, reproduction, genetics and xenotransplantation studies.

The tetravalent heterodimeric soluble TCR protein molecules with enhanced sensitivity for cognate pMHC can be used for the purpose of diagnostics using biological samples obtained directly from human patients. The enhanced sensitivity of the tetravalent heterodimeric soluble TCRs allows detection of potential disease-associated peptides displayed on MHC, which are naturally found to be expressed at low density. These molecules can also be used for patient stratification for enrolling patient onto relevant clinical trials.

In another aspect of the invention, octavalent heterodimeric soluble TCR protein molecules can be used in pharmaceutical preparations for the treatment of various diseases.

In another related aspect to this invention, octavalent heterodimeric soluble TCR protein molecules can be used as a probe for tumour molecular imaging or prepared as a therapeutic protein.

Optionally, the polypeptide (first polypeptide) comprises or consists of a polypeptide disclosed in Table 8. In an example the invention provides a multimer (eg, a dimer, trimer or tetramer, preferably a tetramer) of such a polypeptide. In an example, in Table 8, the multimerization domain (SAM) is a p53 domain (eg, a human p53 domain). In an example, in Table 8, the multimerization domain (SAM) is an orthologue or homologue of a p53 domain (eg, a human p53 domain).

Optionally, the invention provides a polypeptide (eg, said polypeptide or said first polypeptide), wherein the polypeptide comprises or consists of (in N- to C-terminal direction*);
A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;
H. A VH, a CH1, a CH2 a CH3 and a SAM;
I. A VL, a CL, a CH2, a CH3 and a SAM;
J. A dAb; a SAM, a CH2 and a CH3;
K. A scFv; a SAM, a CH2 and a CH3;
L. A VH, a CH1, a SAM, a CH2 and a CH3;
M. A VL, a CL, a SAM, a CH2 and a CH3;
N. A first dAb, a second dAb and a SAM;
O. A VH, a CH1 and a SAM;
P. A VL, a CL and a SAM;
Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.

*In an alternative the components are written in the C- to N-terminal direction.

In an embodiment, polypeptide H, L, O or Q is associated with a second polypeptide, wherein the second polypeptide comprises (in N- to C-terminal direction) VL and CL, wherein the CL is associated with the CH1 of the first polypeptide.

In an embodiment, polypeptide I, M, P or R is associated with a second polypeptide, wherein the second polypeptide comprises (in N- to C-terminal direction) VH and CH1, wherein the CH1 is associated with the CL of the first polypeptide.

In an example, the polypeptide is encoded by a nucleotide sequence disclosed in Table 9. In an example, the polypeptide comprises or consists of an amino acid sequence disclosed in Table 10.

In an example (i) the polypeptide comprises (in N- to C-terminal direction);
A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;
H. A VH, a CH1, a CH2 a CH3 and a SAM;
I. A VL, a CL, a CH2, a CH3 and a SAM;
J. A dAb; a SAM, a CH2 and a CH3;
K. A scFv; a SAM, a CH2 and a CH3;
L. A VH, a CH1, a SAM, a CH2 and a CH3;
M. A VL, a CL, a SAM, a CH2 and a CH3;
N. A first dAb, a second dAb and a SAM;
O. A VH, a CH1 and a SAM;
P. A VL, a CL and a SAM;
Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.
Or (ii) the polypeptide comprises (in C- to N-terminal direction);
A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;
H. A VH, a CH1, a CH2 a CH3 and a SAM;
I. A VL, a CL, a CH2, a CH3 and a SAM;
J. A dAb; a SAM, a CH2 and a CH3;
K. A scFv; a SAM, a CH2 and a CH3;
L. A VH, a CH1, a SAM, a CH2 and a CH3;
M. A VL, a CL, a SAM, a CH2 and a CH3;
N. A first dAb, a second dAb and a SAM;
O. A VH, a CH1 and a SAM;
P. A VL, a CL and a SAM;
Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.

Optionally, the SAM is a tetramerisation domain, eg, a p53 TD.

In an example the first, second, third (when present) and fourth (when present) dAbs have the same antigen binding specificity. In an example the first, second, third (when present) and fourth (when present) dAbs have the same different binding specificity.

In an example the first and second scFvs have the same antigen binding specificity. In an example the first and second scFvs have the same different antigen binding specificity.

In an example the first dAb, second dAb and first scFv have the same antigen binding specificity. In an example the first dAb, second dAb and first scFv have the same different antigen binding specificity.

Herein, where a dAb is provided in the polypeptide, in an alternative there may be provided instead any different type of antigen binding domain, such as a scFv or Fab or non-Ig binding domain (eg, an affibody, avimer or fibronectin domain).

Herein, where a scFv is provided in the polypeptide, in an alternative there may be provided instead any different type of antigen binding domain, such as a dAb or Fab or non-Ig binding domain (eg, an affibody, avimer or fibronectin domain).

Herein, where a Fab is provided in the polypeptide, in an alternative there may be provided instead any different type of antigen binding domain, such as a scFv or dAb or non-Ig binding domain (eg, an affibody, avimer or fibronectin domain).

Each antigen may be any antigen disclosed herein.

In an example, the CH1 (when present), CH2 and CH3 are a human Ig CH1, a CH2 a CH3, eg, a IgG1 CH1, CH2 and CH3. In an example, the CH2 comprises a CH2 domain, the CH3 comprises a CH3 domain and the CH2 comprises a hinge amino acid sequence. In this example, the CH2 comprises (in N- to C-terminal direction) the hinge amino acid sequence and the CH2 domain. In an example the hinge amino acid sequence (i) is a complete hinge; (ii) is a hinge amino acid sequence that is non-functional to dimerise the polypeptide with another such polypeptide; (iii) a hinge amino acid sequence devoid of a hinge core comprising the amino acid motif CXXC (and optionally also devoid of an upper hinge amino acid sequence); or (iv) an upper hinge fused to a lower hinge, but devoid of a hinge core comprising the amino acid motif CXXC; or (v) a lower hinge, but devoid of a hinge core comprising the amino acid motif CXXC (and optionally also devoid of an upper hinge amino acid sequence). Examples of upper, core and lower hinge sequences are disclosed in Table 12. In an example, the CH2 is devoid of a functional hinge region, ie, wherein the hinge region is non-functional to dimerise the polypeptide with another such polypeptide. In an example, the CH2 is devoid of a hinge region. In an example, the CH2 is devoid of a complete hinge region sequence. In an example, the CH2 is devoid of a core hinge region sequence.

In an example, the CH2 comprises (in N- to C- terminal direction) an optional upper hinge region, a lower hinge region and a CH2 domain and wherein the CH2 (and the polypeptide) is devoid of a core hinge region that is functional to dimerise the polypeptide with another said polypeptide.

In an example, the CH2 comprises in N- to C- terminal direction) an optional upper hinge region, a lower hinge region and a CH2 domain and the wherein the CH2 (and the polypeptide) is devoid of a core hinge region amino acid sequence CXXC, wherein X is any amino acid (optionally wherein each amino acid X is selected from a P, R and S).

In an example, the CH2 comprises in N- to C- terminal direction) an amino acid selected from SEQ ID NOs: 163-178 and a CH2 domain and the wherein the CH2 (and the polypeptide) is devoid of a core hinge region amino acid sequence CXXC, wherein X is any amino acid (optionally wherein each amino acid X is selected from a P, R and S).

In an embodiment, the core hinge region amino acid sequence is selected from SEQ ID Nos: 180-182. In an embodiment, the CH2 (an the polyeptide) is devoid of amino acid sequences SEQ ID NOs: 183-187.

In an embodiment, the CH2 domain is a human IgG1 CH2 domain and the core hinge region amino acid sequence is SEQ ID NO: 180. Optionally, any CH1 and CH3 present in the polypeptide are human IgG1 CH1 and CH3 respectively. Optionally, the CH2 (and the polypeptide) is devoid of a core hinge region amino acid sequence CPPC (SEQ ID NO: 180). Optionally, the CH2 (and the polypeptide) is devoid of upper hinge region amino acid sequence EPKSCDKTHT (SEQ ID NO: 183) and core hinge region amino acid sequence CPPC (SEQ ID NO: 180).

In an embodiment, the CH2 domain is a human IgG2 CH2 domain and the core hinge region amino acid sequence is SEQ ID NO: 180. Optionally, any CH1 and CH3 present in the polypeptide are human IgG2 CH1 and CH3 respectively. Optionally, the CH2 (and the polypeptide) is devoid of upper hinge region amino acid sequence ERKCCVE (SEQ ID NO: 184) and core hinge region amino acid sequence CPPC (SEQ ID NO: 180).

In an embodiment, the CH2 domain is a human IgG3 CH2 domain and the core hinge region amino acid sequence is SEQ ID NO: 181. Optionally, any CH1 and CH3 present in the polypeptide are human IgG3 CH1 and CH3 respectively. Optionally, the CH2 (and the polypeptide) is devoid of upper hinge region amino acid sequence ELKTPLGDTTHT (SEQ ID NO: 185) and core hinge region amino acid sequence CPRC (SEQ ID NO: 181). Optionally, alternatively the CH2 (and the polypeptide) is devoid of upper hinge region amino acid sequence EPKSCDTPPP (SEQ ID NO: 186) and core hinge region amino acid sequence CPRC (SEQ ID NO: 181).

In an embodiment, the CH2 domain is a human IgG4 CH2 domain and the core hinge region amino acid sequence is SEQ ID NO: 182. Optionally, any CH1 and CH3 present in the polypeptide are human IgG4 CH1 and CH3 respectively. Optionally, the CH2 (and the polypeptide) is devoid of upper hinge region amino acid sequence ESKYGPP (SEQ ID NO: 187) and core hinge region amino acid sequence CPSC (SEQ ID NO: 182).

In an example, the CH2 of a polypeptide herein is devoid of a core hinge (and optionally also an upper hinge) amino acid sequence. In an example, the CH2 of a polypeptide herein is devoid of a core hinge CXXC amino acid sequence, wherein X is any amino acid, preferably P, R or S, most preferably P. In an example, the CH2 comprises an APELLGGPSV amino acid sequence, or an PAPELLGGPSV amino acid sequence. In an example, the CH2 comprises an APPVAGPSV amino acid sequence, or an PAPPVAGPSV amino acid sequence. In an example, the CH2 comprises an APEFLGGPSV amino acid sequence, or an PAPEFLGGPSV amino acid sequence.

In an example, the CH2 and CH3 of a polypeptide herein are human IgG1 CH2 and CH3 domains, wherein the CH2 is devoid of a core hinge (and optionally also an upper hinge) amino acid sequence, eg, wherein the CH2 is devoid of a CPPC sequence. In an example, the CH2 comprises an APELLGGPSV amino acid sequence, or an EPKSCDKTHT[P]APELLGGPSV amino acid sequence, wherein the bracketed P is optional.

In an example, the CH2 and CH3 of a polypeptide herein are human IgG2 CH2 and CH3 domains, wherein the CH2 is devoid of a core hinge (and optionally also an upper hinge) amino acid sequence, eg, wherein the CH2 is devoid of a CPPC sequence. In an example, the CH2 comprises an APPVAGPSV amino acid sequence, or an ERKCCVE[P]APPVAGPSV amino acid sequence, wherein the bracketed P is optional.

In an example, the CH2 and CH3 of a polypeptide herein are human IgG3 CH2 and CH3 domains, wherein the CH2 is devoid of a core hinge (and optionally also an upper hinge) amino acid sequence, eg, wherein the CH2 is devoid of a CPRC sequence. In an example, the CH2 comprises an APELLGGPSV amino acid sequence, or an ELKTPLGDTTHT[P]APELLGGPSV amino acid sequence, wherein the bracketed P is optional. In an example, the CH2 comprises an EPKSCDTPPP[P]APELLGGPSV amino acid sequence, wherein the bracketed P is optional.

In an example, the CH2 and CH3 of a polypeptide herein are human IgG4 CH2 and CH3 domains, wherein the CH2 is devoid of a core hinge (and optionally also an upper hinge) amino acid sequence, eg, wherein the CH2 is devoid of a CPSC sequence. In an example, the CH2 comprises an APEFLGGPSV amino acid sequence, or an ESKYGPP[P]APEFLGGPSV amino acid sequence, wherein the bracketed P is optional.

When the polypeptide comprises a V-CH1, a CH2 may also be present, but in this case optionally lacking the core hinge region (or at least a sequence selected from CXXC as disclosed herein and SEQ ID Nos: 180-182) and optionally lacking the upper and/or the lower hinge region to prevent F(ab′)2 formation.

### Aspects

By way of example the invention provides the following Aspects, some of which have been exemplified herein. The following Aspects are not to be interpreted as Claims. The Claims start after the Examples section.

1. A polypeptide comprising (in N- to C-terminal direction; or in C- to N-terminal direction)
(a) An immunoglobulin superfamily domain;
(b) An optional linker; and
(c) A self-associating multimerisation domain (SAM) (optionally a self-associating tetramerisation domain (TD)).

In an example, each linker is a peptide linker comprising (or comprising up to, or consisting of) 40, 30, 25, 20, 19, 18, 17, 16, 15 14, 13, 12, 11, 10, 9, 8, 7, 6, 5, 4 or amino acids.

In an alternative, the domain of (a) is a non-Ig domain or comprises a non-Ig scaffold.

In an alternative herein, instead of using a TD or copies of a TD, in an embodiment any other self-associating multimerization domain (SAM) may be used. In an example, the SAM (eg, TD) is a human, dog, cat, horse, monkey (eg, cynomolgus monkey), rodent (eg, mouse or rat), rabbit, bird (eg, chicken) or fish SAM (or TD).

Optionally, the domain of (a) is capable of specifically binding to an antigen selected from PD-L1, PD-1, 4-1BB, CTLA-4, 4-1BB, CD28, TNF alpha, IL17 (eg, IL17A), CD38, VEGF-A, EGFR, IL-6, IL-4, IL-6R, IL-4R (eg, IL-4Ra), OX40, OX40L, TIM-3, CD20, GITR, VISTA, ICOS, Death Receptor 5 (DR5), LAG-3, CD40, CD40L, CD27, HVEM, KRAS, haemagglutinin, transferrin receptor 1, amyloid beta, BACE1, Tau, TDP43, SOD1, Alpha Synculein and CD3.

In an example, the antigen is a peptide-MHC.

In some embodiments (eg, some embodiments of Aspect 16 below or 17 below), the polypeptide comprises at least two binding moieties, eg, two dAbs, two scFvs, or a dAb and a scFv. In an example, these binding moieties bind to the same antigen (eg, an antigen disclosed herein or in the immediately preceding paragraph herein). In another example, the moieties bind to different antigens (eg, an antigen disclosed herein or in the immediately preceding paragraph herein). For example, in Aspect 16B, D, E or F the variable domains or scFvs are capable of specifically binding to the same or different antigens selected from TNF alpha, CD38, IL17a, CD20, PD-1, PD-L1, CTLA-4 and 4-1BB. For example, one of the moieties binds to TNF alpha and the other binds to IL17a; one of the moieties binds to PD-1 and the other binds to 4-1BB; or one of the moieties binds to PD-L1 and the other binds to 4-1BB; one of the moieties binds to PD-1 and the other binds to CTLA-4; or one of the moieties binds to PD-L1 and the other binds to CTLA-4.

2. The polypeptide of any preceding Aspect, wherein the domain of (a) is an antibody variable domain.

For example, a variable domain herein is a VH (eg, comprised by a scFv or a Fab polypeptide chain). In another example, it is a VHH (eg, comprised by a scFv or a Fab polypeptide chain). In another example, it is a humanised VH, humanised VHH or a human VH (eg, comprised by a scFv or a Fab polypeptide chain). In another example, it is a VL (eg, comprised by a scFv or a Fab polypeptide chain). In another example, it is a Vκ. In another example, it is a Vλ.

In another example, the domain of (a) is a TCR variable domain (eg, a TCRα, TCRβ, TCRγ or TCRδ).

In an example the immunoglobulin superfamily domain is an antibody single variable domain (dAb).

3. The polypeptide of any preceding Aspect, wherein the domain of (a) is selected from an antibody single variable domain, a VH and a VL; or wherein the domain is comprised by an scFv.

In an example, a single variable domain herein is a human or humanised dAb or nanobody; or is a camelid VHH domain.

In an example, the domain of (a) is comprised by a single-chain TCR (scTCR).
4. The polypeptide of any preceding Aspect, wherein (a) is joined directly to (c); or wherein (b) is joined directly to (a) and (c).
5. The polypeptide of any preceding Aspect, comprising (in N- to C-terminal direction) the SAM, (d) an optional second linker and (e) a second immunoglobulin superfamily domain.
6. The polypeptide of Aspect 5, wherein the second domain is selected from an antibody single variable domain, a VH and a VL; or wherein the domain is comprised by an scFv.

In an example, the single variable domain is a human or humanised dAb or nanobody; or is a camelid VHH domain.
7. The polypeptide of Aspect 5, wherein the second domain is an antibody single variable domain or an antibody constant domain.
8. The polypeptide of Aspect 7, wherein the constant domain is comprised by an antibody Fc region.
9. The polypeptide of any one of Aspects 5 to 8, wherein (c) is joined directly to (e); or wherein (d) is joined directly to (c) and (e).
10. The polypeptide of any preceding Aspect, wherein the TD is a p53, p63 or p73 TD or a homologue or orthologue thereof; or wherein the TD is a NHR2 TD or a homologue or orthologue thereof.

Optionally, the TD herein is a TD of a protein disclosed in Table 2.
11. The polypeptide of any preceding Aspect, wherein the TD comprises an amino acid sequence that is at least 80% identical to SEQ ID NO: 10 or 126.
12. The polypeptide of any preceding Aspect, comprising (f) an antibody variable domain, an antibody constant region or an antibody Fc region between (a) and (c).
13. The polypeptide of Aspect 12, wherein (f) comprises (i) an antibody CH1 constant domain; or (ii) an antibody Fc region (ie, comprising a CH2-CH3).
14. The polypeptide of Aspect 13, wherein the polypeptide is associated with a second polypeptide, wherein (iii) the second polypeptide comprises an antibody CL constant domain that is paired with the CH1 domain of (i); or (iv) the second polypeptide comprises a second antibody Fc region that is paired with the Fc region of (ii).
15. The polypeptide of Aspect 12, wherein the variable domain of (f) is an antibody single variable domain.
16. The polypeptide of any preceding Aspect, wherein the polypeptide (first polypeptide) comprises or consists of (in N- to C-terminal direction);
   A. A first antibody single variable domain (dAb), an optional linker and said SAM;
   B. A first antibody single variable domain, an optional linker, said SAM and a second antibody single variable domain;
   C. A first scFv, an optional linker and said SAM;
   D. A first scFv, an optional linker, said SAM and a second scFv;
   E. A first antibody single variable domain, an optional linker, said SAM and a first scFv;
   F. A first scFv, an optional linker, said SAM and a first antibody single variable domain;
   G. A first antibody variable domain, an optional first linker, a first antibody constant domain, a second optional linker and said SAM;
   H. Said SAM, an optional linker and a first antibody single variable domain;
   I. Said SAM, an optional linker and a first scFv;
   J. Said SAM, an optional linker, a first antibody constant domain, a second optional linker and a first antibody variable domain; or
   K. Said SAM, an optional linker, a first antibody variable domain, a second optional linker and a first antibody constant domain.

Optionally, each variable domain is a VH or a VL (eg, a Vκ or a Vλ).

Optionally, each domain of the polypeptide herein is a human domain. Optionally, each domain of the polypeptide herein is a human or humanised domain.

17. The polypeptide of any of Aspects 1 to 15, wherein
(i) the polypeptide comprises (in N- to C-terminal direction);
   A. A dAb and a self-associating multimersiation domain (SAM)
   B. A first dAb, a SAM and a second dAb
   C. A first scFv and a SAM;
   D. A first scFv, a SAM and a second scFv;
   E. A first scFv, a SAM and a first dAb;
   F. A first dAb, a CH2, a CH3 and a SAM;
   G. A first scFv, a CH2, a CH3 and a SAM;
   H. A VH, a CH1, a CH2 a CH3 and a SAM;
   I. A VL, a CL, a CH2, a CH3 and a SAM;
   J. A dAb; a SAM, a CH2 and a CH3;
   K. A scFv; a SAM, a CH2 and a CH3;
   L. A VH, a CH1, a SAM, a CH2 and a CH3;
   M. A VL, a CL, a SAM, a CH2 and a CH3;
   N. A first dAb, a second dAb and a SAM;
   O. A VH, a CH1 and a SAM;
   P. A VL, a CL and a SAM;
   Q. A VH, a CH1, a SAM and a first dAb;
   R. A VL, a CL, a SAM and a first dAb;
   S. A first dAb, a second dAb, a SAM and a third dAb;
   T. A first dAb, a second dAb, a SAM and a first scFv;
   U. A first dAb, a second dAb, a SAM, a third dAb and a fourth dAb;
   V. A first dAb, a CH2, a CH3, a SAM and a second dAb;
   W. A first dAb, a CH2, a CH3, a SAM and a first scFv;
   X. A first dAb, a second dAb, a CH2, a CH3 and a SAM;
   Y. A first dAb, a second dAb, a CH2, a CH3, a SAM and a third dAb;
   Z. A first dAb, a second dAb, a CH2, a CH3, a SAM and a first scFv; or
   AA. A first dAb, a second dAb, a CH2, a CH3, a SAM, a third dAb and a fourth dAb;
   Or
(ii) the polypeptide comprises (in C- to N-terminal direction);
   A. A dAb and a self-associating multimersiation domain (SAM)
   B. A first dAb, a SAM and a second dAb
   C. A first scFv and a SAM;
   D. A first scFv, a SAM and a second scFv;
   E. A first scFv, a SAM and a first dAb;
   F. A first dAb, a CH2, a CH3 and a SAM;
   G. A first scFv, a CH2, a CH3 and a SAM;
   H. A VH, a CH1, a CH2 a CH3 and a SAM;
   I. A VL, a CL, a CH2, a CH3 and a SAM;
   J. A dAb; a SAM, a CH2 and a CH3;
   K. A scFv; a SAM, a CH2 and a CH3;
   L. A VH, a CH1, a SAM, a CH2 and a CH3;
   M. A VL, a CL, a SAM, a CH2 and a CH3;
   N. A first dAb, a second dAb and a SAM;
   O. A VH, a CH1 and a SAM;
   P. A VL, a CL and a SAM;
   Q. A VH, a CH1, a SAM and a first dAb;
   R. A VL, a CL, a SAM and a first dAb;
   S. A first dAb, a second dAb, a SAM and a third dAb;
   T. A first dAb, a second dAb, a SAM and a first scFv;
   U. A first dAb, a second dAb, a SAM, a third dAb and a fourth dAb;
   V. A first dAb, a CH2, a CH3, a SAM and a second dAb;
   W. A first dAb, a CH2, a CH3, a SAM and a first scFv;
   X. A first dAb, a second dAb, a CH2, a CH3 and a SAM;
   Y. A first dAb, a second dAb, a CH2, a CH3, a SAM and a third dAb;
   Z. A first dAb, a second dAb, a CH2, a CH3, a SAM and a first scFv; or
   AA. A first dAb, a second dAb, a CH2, a CH3, a SAM, a third dAb and a fourth dAb.

Thus, in some embodiments the polypeptide comprises an antibody Fc region, wherein the Fc comprises the CH2 and CH3 domains.
18. The polypeptide of Aspect 16B, 16D, (i) 17B, (i) 17D, (i) 17N, (i) 17S, (i) 17T, (i) 17U, (i) 17X, (i) 17Y, (i) 17Z, (i) 17AA, (ii) 17B, (ii) 17D, (ii) 17N, (ii) 17S, (ii) 17T, (ii) 17U, (ii) 17X, (ii) 17Y, (ii) 17Z or (ii) 17AA wherein the single variable domains (dAbs) are identical; or wherein the scFvs are identical.
19. The polypeptide of Aspect 16B, 16D, (i) 17B, (i) 17D, (i) 17N, (i) 17S, (i) 17T, (i) 17U, (i) 17X, (i) 17Y, (i) 17Z, (i) 17AA, (ii) 17B, (ii) 17D, (ii) 17N, (ii) 17S, (ii) 17T, (ii) 17U, (ii) 17X, (ii) 17Y, (ii) 17Z or (ii) 17AA wherein the single variable domains are different; or wherein the scFvs are different.
20. The polypeptide of Aspect 16G, 16J, 16K, (i) 17H, (i) 17I, (i) 17L, (i) 17M, (i) 170, (i) 17P, (i) 17Q, (i) 17R, (ii) 17H, (ii) 17I, (ii) 17L, (ii) 17M, (ii) 170, (ii) 17P, (ii) 17Q or (ii) 17R wherein (i) the first variable domain is a VH domain and the first constant domain is a CH1 domain, and optionally the polypeptide is associated with a second polypeptide, wherein the second polypeptide comprises an antibody CL constant domain that is paired with the CH1 domain; (ii) the first variable domain is a VH domain and the first constant domain is a CL domain, and optionally the polypeptide is associated with a second polypeptide, wherein the second polypeptide comprises an antibody CH1 constant domain that is paired with the CL domain; (iii) the first variable domain is a VL domain and the first constant domain is a CH1 domain, and optionally the polypeptide is associated with a second polypeptide, wherein the second polypeptide comprises an antibody CL constant domain that is paired with the CH1 domain; or (iv) the first variable domain is a VL domain and the first constant domain is a CL domain, and optionally the polypeptide is associated with a second polypeptide, wherein the second polypeptide comprises an antibody CH1 constant domain that is paired with the CL domain.
21. The polypeptide of any one of Aspects 16 to 20, comprising an antibody Fc region or a further antibody single variable domain between (v) the first variable domain or scFv and (vi) the SAM, wherein the Fc comprises a CH2 and a CH3.

This further variable domain may be different from the first single variable domain or may have a target binding specificity that is different from the target binding specificity of the first single variable domain or scFv.
22. The polypeptide of any one of Aspects 16 to 21, comprising an antibody Fc region or an antibody single variable domain between (vii) the SAM and (viii) the most C-terminal variable domain, wherein the Fc comprises a CH2 and a CH3.
23. The polypeptide of any one of Aspects 16 to 22, comprising in N- to C-terminal direction an antibody Fc region and the most N-terminal variable domain or scFv, wherein the Fc comprises a CH2 and a CH3.
24. The polypeptide of any one of Aspects 16 to 23, comprising in N- to C-terminal direction the SAM and an antibody Fc region, wherein the Fc comprises a CH2 and a CH3.
25. The polypeptide of any one of Aspects 17 to 24, wherein the CH2 is devoid of an amino acid sequence CXXC or an amino acid sequence selected from SEQ ID NOs: 180-182; and optionally is devoid of amino acid sequences SEQ ID NOs: 183-187.

In an example, the CH2 is a CH2′ disclosed herein. Optionally, the CH2 comprises (in N- to C- terminal direction) an optional upper hinge region, a lower hinge region and a CH2 domain and wherein the CH2 (and the polypeptide) is devoid of a core hinge region that is functional to dimerise the polypeptide with another said polypeptide. Optionally, the CH2 comprises in N- to C- terminal direction) an optional upper hinge region, a lower hinge region and a CH2 domain and the wherein the CH2 (and the polypeptide) is devoid of a core hinge region amino acid sequence CXXC, wherein X is any amino acid (optionally wherein each amino acid X is selected from a P, R and S). Optionally, the CH2 comprises in N- to C- terminal direction) an amino acid selected from SEQ ID NOs: 163-178 and a CH2 domain and the wherein the CH2 (and the polypeptide) is devoid of a core hinge region amino acid sequence CXXC, wherein X is any amino acid (optionally wherein each amino acid X is selected from a P, R and S). In an embodiment, the core hinge region amino acid sequence is selected from SEQ ID Nos: 180-182. In an embodiment, the CH2 (an the polyeptide) is devoid of amino acid sequences SEQ ID NOs: 183-187. In an embodiment, the CH2 domain is a human IgG1 CH2 domain and the core hinge region amino acid sequence is SEQ ID NO: 180. Optionally, any CH1 and CH3 present in the polypeptide are human IgG1 CH1 and CH3 respectively. Optionally, the CH2 (and the polypeptide) is devoid of a core hinge region amino acid sequence CPPC (SEQ ID NO: 180). Optionally, the CH2 (and the polypeptide) is devoid of upper hinge region amino acid sequence EPKSCDKTHT (SEQ ID NO: 183) and core hinge region amino acid sequence CPPC (SEQ ID NO: 180). In an embodiment, the CH2 domain is a human IgG2 CH2 domain and the core hinge region amino acid sequence is SEQ ID NO: 180. Optionally, any CH1 and CH3 present in the polypeptide are human IgG2 CH1 and CH3 respectively. Optionally, the CH2 (and the polypeptide) is devoid of upper hinge region amino acid sequence ERKCCVE (SEQ ID NO: 184) and core hinge region amino acid sequence CPPC (SEQ ID NO: 180). In an embodiment, the CH2 domain is a human IgG3 CH2 domain and the core hinge region amino acid sequence is SEQ ID NO: 181. Optionally, any CH1 and CH3 present in the polypeptide are human IgG3 CH1 and CH3 respectively. Optionally, the CH2 (and the polypeptide) is devoid of upper hinge region amino acid sequence ELKTPLGDTTHT (SEQ ID NO: 185) and core hinge region amino acid sequence CPRC (SEQ ID NO: 181). Optionally, alternatively the CH2 (and the polypeptide) is devoid of upper hinge region amino acid sequence EPKSCDTPPP (SEQ ID NO: 186) and core hinge region amino acid sequence CPRC (SEQ ID NO: 181). In an embodiment, the CH2 domain is a human IgG4 CH2 domain and the core hinge region amino acid sequence is SEQ ID NO: 182. Optionally, any CH1 and CH3 present in the polypeptide are human IgG4 CH1 and CH3 respectively. Optionally, the CH2 (and the polypeptide) is devoid of upper hinge region amino acid sequence ESKYGPP (SEQ ID NO: 187) and core hinge region amino acid sequence CPSC (SEQ ID NO: 182).

26. The polypeptide of any preceding Aspect, wherein the first or each linker is a (G₄S)ₙ linker, wherein n=1, 2, 3, 4, 5, 6, 7, 8, 9 or 10.

In an example, n is 3. In an example, n is 4. In an example, n is 5.
27. The polypeptide of any preceding Aspect, wherein each domain and SAM is a human domain and SAM respectively.
28. The polypeptide of any preceding Aspect, wherein each variable domain or scFv is capable of binding to an antigen.

In an example, the binding antagonises the antigen. In another example, the binding agonises the antigen.

29. The polypeptide of any preceding Aspect, wherein the polypeptide comprises binding specificity for more than one antigen, optionally 2, 3 or 4 different antigens.

For example, the polypeptide comprises at least one anti-CTLA-4 binding domain (eg, dAb or scFv) and at least one anti-4-1BB binding domain. For example, the polypeptide comprises at least one anti-CTLA-4 binding domain (eg, dAb or scFv) and at least one anti-PD-L1 binding domain. For example, the polypeptide comprises at least one anti-CTLA-4 binding domain (eg, dAb or scFv) and at least one anti-PD-1 binding domain. For example, the polypeptide comprises at least one anti-TNF alpha binding domain (eg, dAb or scFv) and at least one anti-IL-17A binding domain.

For example, the polypeptide comprises a first antigen binding domain (eg, a said VH, VL, VHH, dAb, scFv or Fab variable region) that is N-terminal of the SAM and a second antigen binding domain (eg, a said VH, VL, VHH, dAb, scFv or Fab variable region) that is C-terminal of the SAM. In an example, the polypeptide comprises a third antigen binding domain (eg, a said VH, VL, VHH, dAb, scFv or Fab variable region) that is N-terminal of the SAM (eg, and also N-terminal of the first domain; or between the first domain and the SAM); and optionally the polypeptide a fourth antigen binding domain (eg, a said VH, VL, VHH, dAb, scFv or Fab variable region) that is C-terminal of the SAM (eg, and also C-terminal of the second domain; or between the second domain and the SAM).

In an embodiment, the first domain is capable of specifically binding to an immune checkpoint or T-cell co-stimulatory antigen (eg, selected from OX40, GITR, VISTA, CD40, CD28, LAG3 and TIM-3) and the second binding site is capable of specifically binding to an immune checkpoint or T-cell co-stimulatory antigen (eg, selected from OX40, GITR, VISTA, CD40, CD28, LAG3 and TIM-3). In an example, the domains have the same antigen binding specificity. In an example, the domains have the same epitope binding specificity. In an example, the domains have different antigen binding specificity. In an example, the domains have different epitope binding specificity on the same antigen. In an example, the domains bind TNF alpha. In an example, the domains bind CD20. In an example, the domains bind PD-1. In an example, the domains bind PD-L1. In an example, the domains bind CTLA-4.

In an embodiment, the first domain is capable of specifically binding to 4-1BB, PD-1 or PD-L1 and the second binding site is capable of specifically binding to CTLA-4. In an embodiment, the second domain is capable of specifically binding to 4-1BB, PD-1 or PD-L1 and the first binding site is capable of specifically binding to CTLA-4. In an example, the first domain is capable of specifically binding to 4-1BB, the second binding site is capable of specifically binding to CTLA-4, the third binding site is capable of specifically binding to an immune checkpoint or T-cell co-stimulatory antigen (eg, selected from OX40, GITR, VISTA, CD40, CD28, LAG3 and TIM-3) and the fourth binding site is capable of specifically binding to an immune checkpoint or T-cell co-stimulatory antigen (eg, selected from OX40, GITR, VISTA, CD40, CD28, LAG3 and TIM-3). In an example, the first domain is capable of specifically binding to PD-1, the second binding site is capable of specifically binding to CTLA-4, the third binding site is capable of specifically binding to an immune checkpoint or T-cell co-stimulatory antigen (eg, selected from OX40, GITR, VISTA, CD40, CD28, LAG3 and TIM-3) and the fourth binding site is capable of specifically binding to an immune checkpoint or T-cell co-stimulatory antigen (eg, selected from OX40, GITR, VISTA, CD40, CD28, LAG3 and TIM-3). In an example, the first domain is capable of specifically binding to PD-L1, the second binding site is capable of specifically binding to CTLA-4, the third binding site is capable of specifically binding to CTLA-4, PD-L1, CD3 or CD28 and the fourth binding site is capable of specifically binding to an immune checkpoint or T-cell co-stimulatory antigen (eg, selected from OX40, GITR, VISTA, CD40, CD28, LAG3 and TIM-3).

In an embodiment, the first domain is capable of specifically binding to TNF alpha and the second binding site is capable of specifically binding to IL-17 (eg, IL-17A). In an embodiment, the second domain is capable of specifically binding to TNF alpha and the first binding site is capable of specifically binding to IL-17 (eg, IL-17A).

In an example, the polypeptide comprises a cytokine, eg, an IL-2, IL-15 or IL-21. In an example, the cytokine is a truncated cytokine, eg, a truncated IL-2, IL-15 or IL-21. In an example, the cytokine is C-terminal of the SAM (eg, C-terminal of the C-terminal most antigen binding domain). In an example, the cytokine is N-terminal of the SAM (eg, N-terminal of the N-terminal most antigen binding domain). In an embodiment of these examples, the first domain is capable of specifically binding to an immune checkpoint or T-cell co-stimulatory antigen (eg, selected from OX40, GITR, VISTA, CD40, CD28, LAG3 and TIM-3). In an embodiment of these examples, the first domain is capable of specifically binding to 4-1BB, PD-1, PD-L1 or CTLA-4. In an embodiment of these examples, the second domain is capable of specifically binding to an immune checkpoint or T-cell co-stimulatory antigen (eg, selected from OX40, GITR, VISTA, CD40, CD28, LAG3 and TIM-3). In an embodiment of these examples, the second domain is capable of specifically binding to 4-1BB, PD-1, PD-L1 or CTLA-4. In an embodiment of these examples, the third domain is capable of specifically binding to an immune checkpoint or T-cell co-stimulatory antigen (eg, selected from OX40, GITR, VISTA, CD40, CD28, LAG3 and TIM-3). In an embodiment of these examples, the third domain is capable of specifically binding to 4-1BB, PD-1, PD-L1 or CTLA-4. In an embodiment of these examples, the fourth domain is capable of specifically binding to an immune checkpoint or T-cell co-stimulatory antigen (eg, selected from OX40, GITR, VISTA, CD40, CD28, LAG3 and TIM-3). In an embodiment of these examples, the fourth domain is capable of specifically binding to 4-1BB, PD-1, PD-L1 or CTLA-4.

30. A multimer (optionally a tetramer) of a polypeptide according to any preceding Aspect.

In an example, the multimer is a polypeptide dimer.

In an example, the multimer is a polypeptide trimer.

In an example, the multimer is a polypeptide tetramer.
31. The polypeptide or multimer of any preceding Aspect, comprising eukaryotic cell glycosylation.
32. The polypeptide or multimer of Aspect 31, wherein the cell is a HEK293, CHO or Cos cell.
33. The polypeptide or multimer of any preceding Aspect for medical use.
34. A pharmaceutical composition comprising the polypeptide or multimer of any preceding Aspect.
35. A nucleic acid encoding a polypeptide of any one of Aspects 1 to 29 and 31 to 33.
36. A eukaryotic cell or vector comprising the nucleic acid of Aspect 35.
37. A method of binding multiple copies of an antigen, the method comprising combining the copies with a multimer of any one of Aspects 30 to 33, wherein the copies are bound by polypeptides of the multimer, and optionally the method comprising isolating the multimer bound to the antigen copies.
38. The method of Aspect 37 wherein the method is a diagnostic method for detecting the presence of a substance in a sample, wherein the substance comprises the antigen, the method comprising providing the sample (eg, a bodily fluid, food, food ingredient, beverage, beverage ingredient, soil or forensic sample), mixing the sample with multimers according to any one of Aspects 30 to 33 and detecting the binding of multimers to the antigen in the sample.

In an example the bodily fluid is a blood, saliva, semen or urine sample.

In an example, the method is for pregnancy testing or diagnosing a disease or condition in a subject from which the sample has been previously obtained.
39. A method of treating or reducing the risk of a disease or condition in a human or animal subject, the method comprising administering the composition of Aspect 34 to the subject, wherein multimers comprised by the composition specifically bind to a target antigen in the subject, wherein said binding mediates the treatment or reduction in risk.
40. The method of Aspect 39, wherein the antigen is an immune checkpoint or T-cell co-stimulatory antigen (eg, PD-L1, PD-1 or CTLA4); or wherein the antigen is TNF alpha or IL-17A.
41. The method of Aspect 39, wherein the antigen mediates the disease or condition in the subject; and optionally wherein the binding antagonises the antigen.

In another embodiment, the binding agonises the antigen.
42. A composition comprising a plurality of polypeptides according to any one of Aspects 1 to 29 and 31 to 33, wherein at least 90% of the polypeptides are comprised by tetramers of said polypeptides.

Optionally, at least 91, 92, 93, 94, 95, 96, 97, 98 or 99% of the polypeptides are comprised by tetramers of said polypeptides.
43. The composition of Aspect 42, wherein at least 98% of the polypeptides are comprised by tetramers of said polypeptides.
44. The composition of Aspect 42 or 43, wherein the remaining (ie, the balance to 100% of polypeptide) polypeptides are selected from one or more of polypeptide monomers, dimers and trimers.
45. A method of producing a composition (optionally a composition according to any one of Aspects 42 to 44) comprising a plurality of polypeptides according to any one of Aspects 1 to 29 and 31 to 33, the method comprising providing eukaryotic host cells according to Aspect 34, culturing the host cells, and allowing expression and secretion from the cells of tetramers of the polypeptides, and optionally isolating or purifying the tetramers.
Any of these Aspects is combinable with any other disclosure herein, eg, any of the Clauses or Paragraphs.

### Polypeptides & Multimers Comprising Fc

The invention also provides polypeptides and multimers comprising antibody Fc region(s). This is useful, for example, to harness FcRn recycling when administered to a subject, such as a human or animal, which may contribute to a desirable half-life in vivo. Fc regions are also useful for providing Fc effector functions. For example, an IgG1 Fc may be useful when the multimer is used to treat a cancer or where cell killing is desired, eg, by ADCC.

To this end, the invention provides the following:

A polypeptide comprising an antibody Fc region, wherein the Fc region comprises an antibody CH2 and an antibody CH3; and a self-associating multimerisation domain (SAM); wherein the CH2 comprises an antibody hinge sequence and is devoid of a core hinge region.

In an embodiment, the polypeptide comprises an epitope binding site, eg, an antibody VH single variable domain or an antibody VH/VL pair that binds to an epitope. Additionally or alternatively, the polypeptide comprises an epitope which is cognate to an antibody. This is useful, for example, as the polypeptide can form a multimer that binds copies of the antibodies, such as when the multimer is contacted with a sample comprising the antibodies (eg, for medical use as disclosed herein). In this way, for example, the multimer can be used in a method of diagnosis or testing to determine the presence and/or quantity (or relative amount) of the antibody in the sample. As the multimer provides multiple copies of the epitope (at least one for each polypeptide comprised by the multimer), this can be useful to bind many copies of the antibody, which may be present in relatively small amounts in the sample, thereby having the effect of enhancing the chances of detecting (or amplifying) a positive signal denoting presence of the antibody. Thus, assay sensitivity may be enhanced so that relatively rare antibodies in samples can be detected.

Optionally, the CH2 is devoid of (i) a core hinge CXXC amino acid sequence, wherein X is any amino acid or wherein each amino acid X is selected from a P, R and S; and/or (ii) an upper hinge amino acid sequence. For the CH2 is devoid of (i) a core hinge CXXC amino acid sequence, wherein X is any amino acid or wherein each amino acid X is selected from a P, R and S and the Fc does not directly pair with another Fc.

Optionally, the CXXC sequence is selected from SEQ ID NOs: 180-182; or the CH2 is devoid of amino acid sequences SEQ ID NOs: 183-187.

Optionally, the CH2 comprises
A. amino acid sequence APELLGGPSV (SEQ ID NO: 163), or PAPELLGGPSV (SEQ ID NO: 164);
B. amino acid sequence APPVAGPSV (SEQ ID NO: 165), or PAPPVAGPSV (SEQ ID NO: 166);
C. amino acid sequence APEFLGGPSV (SEQ ID NO: 175), or PAPEFLGGPSV (SEQ ID NO: 176);
D. amino acid sequence EPKSCDKTHT[P]APELLGGPSV (SEQ ID NO: 167 or 168), wherein the bracketed P is optional;
E. amino acid sequence ERKCCVE[P]APPVAGPSV (SEQ ID NO: 169 or 170), wherein the bracketed P is optional;
F. amino acid sequence ELKTPLGDTTHT[P]APELLGGPSV (SEQ ID NO: 171 or 172), wherein the bracketed P is optional;
G. amino acid sequence EPKSCDTPPP[P]APELLGGPSV (SEQ ID NO: 173 or 174), wherein the bracketed P is optional; or
H. amino acid sequence ESKYGPP[P]APEFLGGPSV (SEQ ID NO: 177 or 178), wherein the bracketed P is optional.

Optionally, the CH2 comprises (in N- to C- terminal direction) an optional upper hinge region, a lower hinge region and a CH2 domain and wherein the CH2 (and the polypeptide) is devoid of a core hinge region that is functional to dimerise the polypeptide with another said polypeptide. Optionally, the CH2 comprises in N- to C- terminal direction) an optional upper hinge region, a lower hinge region and a CH2 domain and the wherein the CH2 (and the polypeptide) is devoid of a core hinge region amino acid sequence CXXC, wherein X is any amino acid (optionally wherein each amino acid X is selected from a P, R and S). Optionally, the CH2 comprises in N- to C- terminal direction) an amino acid selected from SEQ ID NOs: 163-178 and a CH2 domain and the wherein the CH2 (and the polypeptide) is devoid of a core hinge region amino acid sequence CXXC, wherein X is any amino acid (optionally wherein each amino acid X is selected from a P, R and S). In an embodiment, the core hinge region amino acid sequence is selected from SEQ ID Nos: 180-182. In an embodiment, the CH2 (an the polyeptide) is devoid of amino acid sequences SEQ ID NOs: 183-187. In an embodiment, the CH2 domain is a human IgG1 CH2 domain and the core hinge region amino acid sequence is SEQ ID NO: 180. Optionally, any CH1 and CH3 present in the polypeptide are human IgG1 CH1 and CH3 respectively. Optionally, the CH2 (and the polypeptide) is devoid of a core hinge region amino acid sequence CPPC (SEQ ID NO: 180). Optionally, the CH2 (and the polypeptide) is devoid of upper hinge region amino acid sequence EPKSCDKTHT (SEQ ID NO: 183) and core hinge region amino acid sequence CPPC (SEQ ID NO: 180). In an embodiment, the CH2 domain is a human IgG2 CH2 domain and the core hinge region amino acid sequence is SEQ ID NO: 180. Optionally, any CH1 and CH3 present in the polypeptide are human IgG2 CH1 and CH3 respectively. Optionally, the CH2 (and the polypeptide) is devoid of upper hinge region amino acid sequence ERKCCVE (SEQ ID NO: 184) and core hinge region amino acid sequence CPPC (SEQ ID NO: 180). In an embodiment, the CH2 domain is a human IgG3 CH2 domain and the core hinge region amino acid sequence is SEQ ID NO: 181. Optionally, any CH1 and CH3 present in the polypeptide are human IgG3 CH1 and CH3 respectively. Optionally, the CH2 (and the polypeptide) is devoid of upper hinge region amino acid sequence ELKTPLGDTTHT (SEQ ID NO: 185) and core hinge region amino acid sequence CPRC (SEQ ID NO: 181). Optionally, alternatively the CH2 (and the polypeptide) is devoid of upper hinge region amino acid sequence EPKSCDTPPP (SEQ ID NO: 186) and core hinge region amino acid sequence CPRC (SEQ ID NO: 181). In an embodiment, the CH2 domain is a human IgG4 CH2 domain and the core hinge region amino acid sequence is SEQ ID NO: 182. Optionally, any CH1 and CH3 present in the polypeptide are human IgG4 CH1 and CH3 respectively. Optionally, the CH2 (and the polypeptide) is devoid of upper hinge region amino acid sequence ESKYGPP (SEQ ID NO: 187) and core hinge region amino acid sequence CPSC (SEQ ID NO: 182).

Optionally, the polypeptide comprises an antibody CH1-hinge sequence devoid of core region-CH2-CH3.

Optionally, the CH2 and CH3 comprise
A. human IgG1 CH2 and CH3 domains;
B. human IgG2 CH2 and CH3 domains;
C. human IgG3 CH2 and CH3 domains; or
D. human IgG4 CH2 and CH3 domains.

Optionally, the CH2 and CH3 comprise
(a) human IgG1 CH2 and CH3 domains and the hinge sequence and core hinge region is a human IgG1 hinge sequence and hinge region;
(b) human IgG2 CH2 and CH3 domains and the hinge sequence and core hinge region is a human IgG2 hinge sequence and hinge region;
(c) human IgG3 CH2 and CH3 domains and the hinge sequence and core hinge region is a human IgG31 hinge sequence and hinge region; or
(d) human IgG4 CH2 and CH3 domains and the hinge sequence and core hinge region is a human IgG4 hinge sequence and hinge region

Optionally,
A. the CH2 domain comprises a human IgG1 CH2 domain and the core hinge region amino acid sequence is SEQ ID NO: 180, optionally wherein the CH2 is devoid of upper hinge region amino acid sequence EPKSCDKTHT (SEQ ID NO: 183);
B. the CH2 domain comprises a human IgG2 CH2 domain and the core hinge region amino acid sequence is SEQ ID NO: 180, optionally wherein the CH2 is devoid of upper hinge region amino acid sequence ERKCCVE (SEQ ID NO: 184);
C. the CH2 domain comprises a human IgG3 CH2 domain and the core hinge region amino acid sequence is SEQ ID NO: 181, optionally wherein the CH2is devoid of upper hinge region amino acid sequence ELKTPLGDTTHT (SEQ ID NO: 185) or upper hinge region amino acid sequence EPKSCDTPPP (SEQ ID NO: 186); or
D. the CH2 domain comprises a human IgG4 CH2 domain and the core hinge region amino acid sequence is SEQ ID NO: 182, and optionally wherein the CH2 is devoid of upper hinge region amino acid sequence ESKYGPP (SEQ ID NO: 187).

Optionally, the polypeptide comprises (in N- to C-terminal direction) the Fc region and the SAM, the Fc region comprising (in N- to C-terminal direction) the hinge sequence, a CH2 domain and a CH3 domain.

Optionally, the polypeptide comprises one or more epitope binding sites, eg, an antibody variable domain that is capable of specifically binding to a first epitope. Optionally, the first epitope is comprised by an antigen (eg, a human antigen) selected from the group consisting of ABCF1; ACVR1; ACVR1B; ACVR2; ACVR2B; ACVRL1; ADORA2A; Aggrecan; AGR2; AICDA; AWI; AIG1; AKAP1; AKAP2; AIYIH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BM Pl; BMP2; BMP3B (GDFIO); BMP4; BMP6; BM P8; BMPRIA; BMPRIB; BM PR2; BPAG1 (plectin); BRCA1; CI9orflO (IL27w); C3; C4A; C5; C5R1; CANT1; CASP1; CASP4; CAV1; CCBP2 (D6 / JAB61); CCL1 (1-309); CCL11 (eotaxin); CCL13 (MCP-4); CCL15 (MIP-id); CCL16 (HCC-4); CCL17 (TARC); CCL18 (PARC); CCL19 (M IP-3b); CCL2 (MCP-1); MCAF; CCL20 (MIP-3a); CCL21 (MIP-2); SLC; exodus-2; CCL22 (MDC / STC-1); CCL23 (M PIF-1); CCL24 (MPIF-2 I eotaxin-2); CCL25 (TECK); CCL26 (eotaxin-3); CCL27 (CTACK /ILC) ; CCL28; CCL3 (MIP-la); CCL4 (M IP-lb); CCL5 (RANTES); CCL7 (MCP-3); CCL8 (mcp-2); CCNA1; CCNA2; CCND1; CCNE1; CCNE2; CCR1 (CKR1 / HM145); CCR2 (mcp-1RB / RA);CCR3 (CKR3 / CMKBR3); CCR4; CCR5 (CM KBR5 / ChemR13); CCR6 (CMKBR6 / CKR-L3 / STRL22 / DRY6); CCR7 (CKR7 / EBI1); CCR8 (CM KBR8 / TER1 / CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p2IWapl/Cipl); CDKN1B (p27Kipl); CDKNIC; CDKN2A (pl6INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYDi) ; CX3CR1 (V28); CXCL1 (GROl); CXCLIO (IP-10); CXCL11 (1-TAC / IP-9); CXCL12 (SDF1); CXCL13; CXCL14; CXCL16; CXCL2 (GR02); CXCL3 (GR03); CXCL5 (ENA-78 I LIX); CXCL6 (GCP-2); CXCL9 (MIG); CXCR3 (GPR9/CKR-L2); CXCR4; CXCR6 (TYMSTR ISTRL33 I Bonzo); CYB5; CYC1; CYSLTR1; DAB2IP; DES; DKFZp451J0118; DNCL1; DPP4; E2F1; ECGF1; EDG1; EFNAI; EFNA3; EFNB2; EGF; EGFR; ELAC2; ENG; EN01; EN02; EN03; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FILl (EPSILON); FILl (ZETA); FU12584; FU25530; FLRTl (fibronectin); FLTl; FOS; FOSLl (FRA-I); FY (DARC); GABRP (GABAa); GAGEB1; GAGEC1; GALNAC4S-65T; GATA3; GDF5; GFI1; GGT1; GM-CSF; GNAS1; GNRHl; GPR2 (CCRIO); GPR31; GPR44; GPR81 (FKSG80); GRCCIO (CIO); GRP; GSN (Gelsolin); GSTPl; HAVCR2; HDAC4; EDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; 1D2; IFN-a; IFNA1; IFNA2; IFNA4; IFNA5; IFNA6; IFNA7; IFNB1; IFNgamma; TFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; 1L13; IL13RA1; IL13RA2; 1L14; 1L15; IL15RA; IL16; 1L17; IL17B; IL17C; IL17R; 1L18; IL18BP; IL18R1; IL18RAP; 1L19; ILIA; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1;IL1RL2 IL1RN; 1L2; 1L20; IL20RA; IL21R; 1L22; 1L22R; 1L22RA2; 1L23; 1L24; 1L25; 1L26; 1L27; 1L28A; 1L28B; 1L29; IL2RA; IL2RB; IL2RG; 1L3; 1L30; IL3RA; 1L4; IL4R; 1L5; IL5RA; 1L6; IL6R; IL6ST (glycoprotein 130); 1L7; TL7R; 1L8; IL8RA; IL8RB; IL8RB; 1L9; IL9R; ILK; INHA; INHBA; INSL3; INSL4; IRAKI; IRAK2; ITGA1; ITGA2; 1TGA3; ITGA6 (a6 integrin); ITGAV; ITGB3; ITGB4 (b 4 integrin); JAG1; JAK1; JAK3; JUN; K6HF; KAI1; KDR; MTLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; M IB1; midkine; M IF; M IP-2; MK167 (Ki-67); MMP2; M MP9; MS4A1; MSMB; MT3 (metallothionectin-ifi); MTSS 1; M UC 1 (mucin); MYC; MYD88; NCK2; neurocan; NFKB 1; NFKB2; NGFB (NGF); NGFR; NgR-Lingo; NgR-Nogo66 (Nogo); NgR-p75; NgR-Troy; NM E1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p2IRac2); RARB; RGS1; RGS13; RGS3; RNF110 (ZNF144); ROB02; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINIA3; SERPINB5 (maspin); SERPINE1 (PAT-i); SERPINF1; SHBG; SLA2; SLC2A2; SLC33A1; SLC43A1; SLIT2; SPPl; SPRRIB (Spri); ST6GAL1; STABl; STAT6; STEAP; STEAP2; TB4R2; TBX21; TCPIO; TDGF1; TEK; TGFA; TGFB1; TGFB1I1; TGFB2; TGFB3; TGFBI; TGFBR1; TGFBR2; TGFBR3; TH1L; THBS1(thrombospondin-1); THBS2; THBS4; THPO; TIE (Tie-i); T]MP3; tissue factor; TLRIO; TLR2; TLR3; TLR4; TLR5; TLR6; TLR7; TLR8; TLR9; TNF; TNF-a; TNFAIP2 (B94); TNFAIP3; TNFRSF1 1A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF1 1 (TRANCE); TNFSF12 (AP03L); TNFSF13 (April); TNFSF13B; TNFSF14 (HVEM-L); TNFSF1 5 (VEGI); TNFSF1 8; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM 1; TPM2; TRADD; TRAF1; TRAF2; TRAF3; TRAF4; TRAF5; TRAF6; TREM 1; TREM2; TRPC6; TSLP; TWEAK; VEGF; VEGFB; VEGFC; versican; VHL C5; VLA-4; XCL1 (lymphotactin); XCL2 (SCM-lb); XCR1 (GPR5 / CCXCR1); YY1; and ZFPM2. Optionally, the second epitope (as discussed below) is comprised by the same antigen as the first epitope (eg, comprised by the same antigen molecule). In another example, the second antigen is comprised by said group. In an example, the first and second epitopes are comprised by different antigens selected from said group.

For example, the polypeptide has 1, 2, 3, 4 or 5 epitope binding sites (optionally wherein the polypeptide comprises 2 or more binding sites (eg, single variable domains) that bind to different epitopes, or wherein the polypeptide binding sites are identical). In an embodiment, the SAM is a TD (eg, a p53 TD, such as a human p53 TD) and the polypeptide has 2, 3 or 4 binding sites, such as 3 sites or such as 4 sites. Preferably, the polypeptide has 3 binding sites. Preferably, the polypeptide has 4 binding sites. For example, the binding sites each binds TNF alpha (eg, wherein the binding sites are identical, eg, identical antibody single variable domains).

In an example, the multimer is an octavalent bispecific multimer comprising 4 copies of an anti-PD-L1binding site (eg, dAb) and 4 copies of an anti-4-1BB binding site (eg, dAb).

In an example, the multimer is a tetravalent multimer comprising copies of an anti-PD-L1 binding site (eg, dAb). In an example, the multimer is an octavalent multimer comprising copies of an anti-PD-L1 binding site (eg, dAb). In an example, the multimer is a 12-valent multimer comprising copies of an anti-PD-L1 binding site (eg, dAb). In an example, the multimer is a 16-valent multimer comprising copies of an anti-PD-L1 binding site (eg, dAb). In an example, the anti-PD-L1 binding site comprises an avelumab or atezolizumab binding site that specifically binds to PD-L1. Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-PD-1 binding site (eg, dAb). In an example, the multimer is an octavalent multimer comprising copies of an anti-PD-1 binding site (eg, dAb). In an example, the multimer is a 12-valent multimer comprising copies of an anti-PD-1 binding site (eg, dAb). In an example, the multimer is a 16-valent multimer comprising copies of an anti-PD-1 binding site (eg, dAb). In an example, the anti- PD-1 binding site comprises a nivolumab or pembrolizumab binding site that specifically binds to PD-1. Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-DR5 (Death Receptor 5) binding site (eg, dAb). In an example, the multimer is an octavalent multimer comprising copies of an anti-DR5 binding site (eg, dAb). In an example, the multimer is a 12-valent multimer comprising copies of an anti-DR5 binding site (eg, dAb). In an example, the multimer is a 16-valent multimer comprising copies of an anti-DR5 binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-OX40 or OX40L binding site (eg, dAb). In an example, the multimer is an octavalent multimer comprising copies of an anti-OX40 or OX40L binding site (eg, dAb). In an example, the multimer is a 12-valent multimer comprising copies of an anti- OX40 or OX40L binding site (eg, dAb). In an example, the multimer is a 16-valent multimer comprising copies of an anti- OX40 or OX40L binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-glucocorticoid-induced tumor necrosis factor receptor (GITR) binding site (eg, dAb). In an example, the multimer is an octavalent multimer comprising copies of an anti-GITR binding site (eg, dAb). In an example, the multimer is a 12-valent multimer comprising copies of an anti-GITR binding site (eg, dAb). In an example, the multimer is a 16-valent multimer comprising copies of an anti-GITR binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-antibody kappa light chain (KLC) binding site (eg, dAb). In an example, the multimer is an octavalent multimer comprising copies of an anti-KLC binding site (eg, dAb). In an example, the multimer is a 12-valent multimer comprising copies of an anti-KLC binding site (eg, dAb). In an example, the multimer is a 16-valent multimer comprising copies of an anti-KLC binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-VEGF binding site (eg, dAb). In an example, the multimer is an octavalent multimer comprising copies of an anti-VEGF binding site (eg, dAb). In an example, the multimer is a 12-valent multimer comprising copies of an anti-VEGF binding site (eg, dAb). In an example, the multimer is a 16-valent multimer comprising copies of an anti-VEGF binding site (eg, dAb). In an example, the anti-VEGF binding site comprises a VEGF receptor domain that specifically binds to VEGF (eg, a VEGF binding site of human flt (eg, flt-1) or KDR, eg, Ig domain 2 from VEGFR1 or Ig domain 3 from VEGFR2)). In an example, the anti-VEGF binding site comprises an aflibercept, bevacizumab or ranibizumab binding site that specifically binds to VEGF. Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-TNF alpha binding site (eg, dAb). In an example, the multimer is a tetravalent multimer comprising copies of an anti-TNF alpha binding site (eg, dAb). In an example, the multimer is an octavalent multimer comprising copies of an anti-TNF alpha binding site (eg, dAb). In an example, the multimer is a 12-valent multimer comprising copies of an anti-TNF alpha binding site (eg, dAb). In an example, the multimer is a 16-valent multimer comprising copies of an anti-TNF alpha binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

Optionally, the variable domain is selected from an antibody single variable domain, a VH and a VL; or wherein the domain is comprised by an scFv. Optionally, the domain is comprised by an antibody VH/VL pair that binds to said first epitope. In an example, epitope binding herein is specific binding as herein defined.

Optionally, the polypeptide comprises (in N- to C-terminal direction)
A. the variable domain, the SAM and the Fc region;
B. the Fc region, the SAM and the variable domain;
C. the variable domain, the Fc region and the SAM;
D. the SAM, the variable domain and the Fc region; or
E. the SAM, the Fc region and the variable domain.

Optionally, the polypepide comprises a second antibody variable domain N- or C-terminal to the SAM, wherein the second variable domain is capable of specifically binding to a second epitope, wherein the first and second epitopes are identical or different.

Optionally, the SAM is a self-associating tetramerisation domain (TD); optionally wherein the TD is a p53, p63 or p73 TD or a homologue or orthologue thereof; or wherein the TD is a NHR2 TD or a homologue or orthologue thereof; or wherein the TD comprises an amino acid sequence that is at least 80, 85, 90, 91, 92, 93, 94, 95, 96, 97, 98 or 99% identical to SEQ ID NO: 10 or 126.

Optionally,
(i) the polypeptide comprises (in N- to C-terminal direction);
   A. A first antibody single variable domain (dAb), an optional linker and said SAM;
   B. A first antibody single variable domain, an optional linker, said SAM and a second antibody single variable domain;
   C. A first scFv, an optional linker and said SAM;
   D. A first scFv, an optional linker, said SAM and a second scFv;
   E. A first antibody single variable domain, an optional linker, said SAM and a first scFv;
   F. A first scFv, an optional linker, said SAM and a first antibody single variable domain;
   G. A first antibody variable domain, an optional first linker, a first antibody constant domain, a second optional linker and said SAM;
   H. Said SAM, an optional linker and a first antibody single variable domain;
   I. Said SAM, an optional linker and a first scFv;
   J. Said SAM, an optional linker, a first antibody constant domain, a second optional linker and a first antibody variable domain; or
   K. Said SAM, an optional linker, a first antibody variable domain, a second optional linker and a first antibody constant domain;
   Or
(ii) the polypeptide comprises (in N- to C-terminal direction);
   A. A dAb and the SAM;
   B. A first dAb, the SAM and a second dAb;
   C. A first scFv and the SAM;
   D. A first scFv, the SAM and a second scFv;
   E. A first scFv, the SAM and a first dAb;
   F. A first dAb, the Fc region and the SAM;
   G. A first scFv, the Fc region and the SAM;
   H. A VH, a CH1, the Fc region and the SAM;
   I. A VL, a CL, the Fc region and the SAM;
   J. A dAb; the SAM and the Fc region;
   K. A scFv; the SAM and the Fc region;
   L. A VH, a CH1, the SAM and the Fc region;
   M. A VL, a CL, the SAM and the Fc region;
   N. A first dAb, a second dAb and the SAM;
   O. A VH, a CH1 and the SAM;
   P. A VL, a CL and the SAM;
   Q. A VH, a CH1, the SAM and a first dAb;
   R. A VL, a CL, the SAM and a first dAb;
   S. A first dAb, a second dAb, the SAM and a third dAb;
   T. A first dAb, a second dAb, the SAM and a first scFv;
   U. A first dAb, a second dAb, the SAM, a third dAb and a fourth dAb;
   V. A first dAb, the Fc region, the SAM and a second dAb;
   W. A first dAb, the Fc region, the SAM and a first scFv;
   X. A first dAb, a second dAb, the Fc region and the SAM;
   Y. A first dAb, a second dAb, the Fc region, the SAM and a third dAb;
   Z. A first dAb, a second dAb, the Fc region, the SAM and a first scFv; or
   AA. A first dAb, a second dAb, the Fc region, the SAM, a third dAb and a fourth dAb;
   Or
(iii) the polypeptide comprises (in C- to N-terminal direction);
   A. A dAb and the SAM;
   B. A first dAb, the SAM and a second dAb;
   C. A first scFv and the SAM;
   D. A first scFv, the SAM and a second scFv;
   E. A first scFv, the SAM and a first dAb;
   F. A first dAb, the Fc region and the SAM;
   G. A first scFv, the Fc region and the SAM;
   H. A VH, a CH1, the Fc regionand the SAM;
   I. A VL, a CL, the Fc region and the SAM;
   J. A dAb; the SAM and the Fc region;
   K. A scFv; the SAM and the Fc region;
   L. A VH, a CH1, the SAM and the Fc region;
   M. A VL, a CL, the SAM and the Fc region;
   N. A first dAb, a second dAb and the SAM;
   O. A VH, a CH1 and the SAM;
   P. A VL, a CL and the SAM;
   Q. A VH, a CH1, the SAM and a first dAb;
   R. A VL, a CL, the SAM and a first dAb;
   S. A first dAb, a second dAb, the SAM and a third dAb;
   T. A first dAb, a second dAb, the SAM and a first scFv;
   U. A first dAb, a second dAb, the SAM, a third dAb and a fourth dAb;
   V. A first dAb, the Fc region, the SAM and a second dAb;
   W. A first dAb, the Fc region, the SAM and a first scFv;
   X. A first dAb, a second dAb, the Fc region and the SAM;
   Y. A first dAb, a second dAb, the Fc region, the SAM and a third dAb;
   Z. A first dAb, a second dAb, the Fc region, the SAM and a first scFv; or
   AA. A first dAb, a second dAb, the Fc region, the SAM, a third dAb and a fourth dAb.

Optionally, any single variable domain or dAb herein is a Nanobody™ or a Camelid VHH (eg, a humanised Camelid VHH). For example, a variable domain, such as a dAb (AKA antibody single variable domain) herein is a VH (eg, comprised by a scFv or a Fab polypeptide chain). In another example, it is a VHH (eg, comprised by a scFv or a Fab polypeptide chain). In another example, it is a humanised VH, humanised VHH or a human VH (eg, comprised by a scFv or a Fab polypeptide chain). In another example, it is a VL (eg, comprised by a scFv or a Fab polypeptide chain). In another example, it is a Vκ. In another example, it is a Vλ.

In an embodiment, each polypeptide of the multimer is paired with a copy of a further polypeptide, wherein the further polypeptide comprises an antibody light chain constant region (eg, a Cκ or a Cλ) that pairs with the Fc of the first polypeptide. In an example, the first polypeptide comprises an antibody VH domain, the further polypeptide comprises an antibody VL domain (eg, a Vκ or a Vλ), wherein the VH and VL form an epitope binding site. In this way, the multimer may be a multimer of Fab-like structures, such as comprising multiple copies of an adalimumab (Humira) or avelumab (Bavencio) binding site as exemplified in the Examples below. All of the antibody domains in such a multimer may, for example, be human, and optionally the SAM is a human domain. When the SAM is a TD (eg, a p53 TD), the multimer comprises a tetramer of the VH/VL epitope binding sites. The first polypeptide or the further polypetide may comprise a second epitope binding site, for example, wherein the multimer is octavalent. When the 8 binding sites may bind the same antigen; alternatively the 4 VH/VL binding sites bind a first antigen and the other 4 binding sites bind to a second antigen, wherein the antigens are different. Thus, the multimer may be octavalent and bispecific. If the first or further polypetide comprises yet another antigen binding site, the multimer may be 12-valent (and, eg, monospecific, bispecific or trispecific for antigen binding). If the first or further polypetide comprises yet another antigen binding site, the multimer may be 16-valent (and, eg, monospecific, bispecific, trispecific or tetraspecific for antigen binding).

The invention further provides:-
A multimer (optionally a tetramer) of a polypeptide according to the invention; optionally wherein the multimer is for medical use. In an example, the medical use herein is the treatment or prevention of a cancer, autoimmune disease or condition or any other disease or condition disclosed herein.
A multimer of a plurality of antibody Fc regions, wherein each Fc is comprised by a respective polypeptide and is unpaired with another Fc region; optionally wherein the multimer is for medical use. Optionally, each polypeptide comprises an epitope binding domain or site as disclosed herein.
A pharmaceutical composition comprising the polypeptide or multimer of the invention.
A nucleic acid encoding a polypeptide of the invention; optionally wherein the nucleic acid is comprised by a eukaryotic cell or a vector.
A method of binding multiple copies of an antigen, the method comprising combining the copies with a multimer of or the composition of the invention, wherein the copies are bound by polypeptides of the multimer, and optionally the method comprising isolating the multimer bound to the antigen copies. In an example, the multimer is contacted with a sample comprising the copies of the antigen and copies of the antigen are sequestered in the sample by binding to the multimer. For example, the multimer is administered to a human or animal patient (or an environment is exposed to the multimer) and antigen copies are sequestered in the human (eg, for said medical use), animal (eg, for said medical use) or environment. For example, the environment is comprised by a soil, water source, waterway or industrial fluid, eg, for environmental remediation, such as where the antigen is comprised by an environmental pollutant or contaminant. In an example, the method is for purifying the sample or for isolating antigen comprised by the sample.
A method of treating or reducing the risk of a disease or condition in a human or animal subject, the method comprising administering the composition of the invention to the subject, wherein multimers comprised by the composition specifically bind to a target antigen in the subject, wherein said binding mediates the treatment or reduction in risk of the disease or condition.
A method of producing a composition comprising a plurality of polypeptides according to the invention, wherein the SAM is a self-associating tetramerisation domain (TD), the method comprising providing eukaryotic host cells according to the invention, culturing the host cells, and allowing expression and secretion from the cells of tetramers of the polypeptides, and optionally isolating or purifying the tetramers.

### Paragraphs

The invention provides the following Paragraphs. The following Paragraphs are not to be interpreted as Claims. The Claims start after the Examples section.
1. A polypeptide (optionally according any polypeptide herein) comprising
   (a) An antibody Fc region, wherein the Fc region comprises an antibody CH2 domain and an antibody CH3 domain; and
   (b) A self-associating multimerisation domain (SAM);
   wherein the CH2 is devoid of a core hinge CXXC amino acid sequence, wherein X is any amino acid.
2. The polypeptide of Paragraph 1, wherein each amino acid X is selected from a P, R and S.
3. The polypeptide of Paragraph 1 or 2, wherein the CH2 is devoid of a complete upper hinge sequence.
4. The polypeptide of any preceding Paragraph, wherein the CH2 comprises
   (a) an APELLGGPSV amino acid sequence, or an PAPELLGGPSV amino acid sequence;
   (b) an APPVAGPSV amino acid sequence, or an PAPPVAGPSV amino acid sequence; or
   (c) an APEFLGGPSV amino acid sequence, or an PAPEFLGGPSV amino acid sequence.
5. The polypeptide of any preceding Paragraph, wherein the CH2 and CH3 are
   (a) human IgG1 CH2 and CH3 domains;
   (b) human IgG2 CH2 and CH3 domains;
   (c) human IgG3 CH2 and CH3 domains; or
   (d) human IgG4 CH2 and CH3 domains.
6. The polypeptide of Paragraph 5(a) or (b), wherein CH2 is devoid of a CPPC sequence; or the polypeptide of Paragraph 5(c) wherein the CH2 is devoid of a CPRC sequence; or the polypeptide of Paragraph 5(d) wherein the CH2 is devoid of a CPSC sequence.
7. The polypeptide of any one of Paragraphs 1 to 4, wherein the CH2 comprises
   (a) an APELLGGPSV amino acid sequence;
   (b) an EPKSCDKTHT[P]APELLGGPSV amino acid sequence, wherein the bracketed P is optional;
   (c) an APPVAGPSV amino acid sequence;
   (d) an ERKCCVE[P]APPVAGPSV amino acid sequence, wherein the bracketed P is optional;
   (e) an ELKTPLGDTTHT[P]APELLGGPSV amino acid sequence, wherein the bracketed P is optional;
   (f) an EPKSCDTPPP[P]APELLGGPSV amino acid sequence, wherein the bracketed P is optional; or
   (g) an APEFLGGPSV amino acid sequence; or
   (h) an ESKYGPP[P]APEFLGGPSV amino acid sequence, wherein the bracketed P is optional.
8. The polypeptide of any preceding Paragraph, wherein the CH2 is devoid of a sequence selected from CXXC disclosed herein and SEQ ID Nos: 180-182.
9. The polypeptide of any preceding Paragraph, wherein the polypeptide comprises an antibody variable domain that is capable of specifically binding to a first epitope.
10. The polypeptide of Paragraph 9, wherein the variable domain selected from an antibody single variable domain, a VH and a VL; or wherein the domain is comprised by an scFv.
11. The polypeptide of any preceding Paragraph, wherein the Fc region is 3′ of the SAM.
12. The polypeptide of Paragraph 9, 10 or 11, wherein
   (a) the variable domain is 5′ of the SAM and the Fc region is 3′ of the SAM;
   (b) the variable domain is 3′ of the SAM and the Fc region is 5′ of the SAM;
   (c) the variable domain and Fc are 5′ of the SAM; or
   (d) the variable domain and Fc are 3′ of the SAM.
13. The polypeptide of Paragraph 12 comprising a second variable domain 5′ or 3′ of the SAM, wherein the second variable domain is capable of specifically binding to a second epitope, wherein the first and second epitopes are identical or different.
14. The polypeptide of paragraph 13, wherein the epitopes are different epitopes of the same antigen, or are epitopes of different antigens.
15. The polypeptide of Paragraph 13 or 14, wherein the second variable domain is selected from an antibody single variable domain, a VH and a VL; or wherein the domain is comprised by an scFv.
16. The polypeptide of Paragraph 15, wherein the second variable domain is an antibody single variable domain or an antibody constant domain.
17. The polypeptide of any preceding Paragraph, wherein the SAM is a self-associating tetramerisation domain (TD).
18. The polypeptide of Paragraph 17, wherein the TD is a p53, p63 or p73 TD or a homologue or orthologue thereof; or wherein the TD is a NHR2 TD or a homologue or orthologue thereof.
19. The polypeptide of Paragraph 17 or 18, wherein the TD comprises an amino acid sequence that is at least 80% identical to SEQ ID NO: 10 or 126.
20. The polypeptide of any preceding Paragraph, comprising an antibody CH1 constant domain, optionally a CH1-CH2-CH3, wherein the CH2 and CH3 are comprised by said Fc region.
21. The polypeptide of any preceding Paragraph, wherein the polypeptide (first polypeptide) comprises or consists of (in N- to C-terminal direction);
   A. A first antibody single variable domain (dAb), an optional linker and said SAM;
   B. A first antibody single variable domain, an optional linker, said SAM and a second antibody single variable domain;
   C. A first scFv, an optional linker and said SAM;
   D. A first scFv, an optional linker, said SAM and a second scFv;
   E. A first antibody single variable domain, an optional linker, said SAM and a first scFv;
   F. A first scFv, an optional linker, said SAM and a first antibody single variable domain;
   G. A first antibody variable domain, an optional first linker, a first antibody constant domain, a second optional linker and said SAM;
   H. Said SAM, an optional linker and a first antibody single variable domain;
   I. Said SAM, an optional linker and a first scFv;
   J. Said SAM, an optional linker, a first antibody constant domain, a second optional linker and a first antibody variable domain; or
   K. Said SAM, an optional linker, a first antibody variable domain, a second optional linker and a first antibody constant domain.
22. The polypeptide of any of Paragraphs 1 to 20, wherein
   (i) the polypeptide comprises (in N- to C-terminal direction);
      A. A dAb and the self-associating multimersiation domain (SAM);
      B. A first dAb, the SAM and a second dAb;
      C. A first scFv and the SAM;
      D. A first scFv, the SAM and a second scFv;
      E. A first scFv, the SAM and a first dAb;
      F. A first dAb, the Fc region and the SAM;
      G. A first scFv, the Fc region and the SAM;
      H. A VH, a CH1, the Fc region and the SAM;
      I. A VL, a CL, the Fc region and the SAM;
      J. A dAb; the SAM and the Fc region;
      K. A scFv; the SAM and the Fc region;
      L. A VH, a CH1, the SAM and the Fc region;
      M. A VL, a CL, the SAM and the Fc region;
      N. A first dAb, a second dAb and the SAM;
      O. A VH, a CH1 and the SAM;
      P. A VL, a CL and the SAM;
      Q. A VH, a CH1, the SAM and a first dAb;
      R. A VL, a CL, the SAM and a first dAb;
      S. A first dAb, a second dAb, the SAM and a third dAb;
      T. A first dAb, a second dAb, the SAM and a first scFv;
      U. A first dAb, a second dAb, the SAM, a third dAb and a fourth dAb;
      V. A first dAb, the Fc region, the SAM and a second dAb;
      W. A first dAb, the Fc region, the SAM and a first scFv;
      X. A first dAb, a second dAb, the Fc region and the SAM;
      Y. A first dAb, a second dAb, the Fc region, the SAM and a third dAb;
      Z. A first dAb, a second dAb, the Fc region, the SAM and a first scFv; or
      AA. A first dAb, a second dAb, the Fc region, the SAM, a third dAb and a fourth dAb;
      Or
   (ii) the polypeptide comprises (in C- to N-terminal direction);
      A. A dAb and a self-associating multimersiation domain (SAM);
      B. A first dAb, the SAM and a second dAb;
      C. A first scFv and the SAM;
      D. A first scFv, the SAM and a second scFv;
      E. A first scFv, the SAM and a first dAb;
      F. A first dAb, the Fc region and the SAM;
      G. A first scFv, the Fc region and the SAM;
      H. A VH, a CH1, the Fc regionand the SAM;
      I. A VL, a CL, the Fc region and the SAM;
      J. A dAb; the SAM and the Fc region;
      K. A scFv; the SAM and the Fc region;
      L. A VH, a CH1, the SAM and the Fc region;
      M. A VL, a CL, the SAM and the Fc region;
      N. A first dAb, a second dAb and the SAM;
      O. A VH, a CH1 and the SAM;
      P. A VL, a CL and the SAM;
      Q. A VH, a CH1, the SAM and a first dAb;
      R. A VL, a CL, the SAM and a first dAb;
      S. A first dAb, a second dAb, the SAM and a third dAb;
      T. A first dAb, a second dAb, the SAM and a first scFv;
      U. A first dAb, a second dAb, the SAM, a third dAb and a fourth dAb;
      V. A first dAb, the Fc region, the SAM and a second dAb;
      W. A first dAb, the Fc region, the SAM and a first scFv;
      X. A first dAb, a second dAb, the Fc region and the SAM;
      Y. A first dAb, a second dAb, the Fc region, the SAM and a third dAb;
      Z. A first dAb, a second dAb, the Fc region, the SAM and a first scFv; or
      AA. A first dAb, a second dAb, the Fc region, the SAM, a third dAb and a fourth dAb.
23. The polypeptide of Paragraph 21B, 21D, (i) 22B, (i) 22D, (i) 22N, (i) 22S, (i) 22T, (i) 22U, (i) 22X, (i) 22Y, (i) 22Z, (i) 22AA, (ii) 22B, (ii) 22D, (ii) 22N, (ii) 22S, (ii) 22T, (ii) 22U, (ii) 22X, (ii) 22Y, (ii) 22Z or (ii) 22AA wherein the single variable domains (dAbs) are identical; or wherein the scFvs are identical.
24. The polypeptide of Paragraph 21B, 21D, (i) 22B, (i) 22D, (i) 22N, (i) 22S, (i) 22T, (i) 22U, (i) 22X, (i) 22Y, (i) 22Z, (i) 22AA, (ii) 22B, (ii) 22D, (ii) 22N, (ii) 22S, (ii) 22T, (ii) 22U, (ii) 22X, (ii) 22Y, (ii) 22Z or (ii) 22AA wherein the single variable domains are different; or wherein the scFvs are different.
25. The polypeptide of Paragraph 21G, 21J, 21K, (i) 22H, (i) 22I, (i) 22L, (i) 22M, (i) 22O, (i) 22P, (i) 22Q, (i) 22R, (ii) 22H, (ii) 22I, (ii) 22L, (ii) 22M, (ii) 22O, (ii) 22P, (ii) 22Q or (ii) 22R wherein (i) the first variable domain is a VH domain and the first constant domain is a CH1 domain, and optionally the polypeptide is associated with a second polypeptide, wherein the second polypeptide comprises an antibody CL constant domain that is paired with the CH1 domain; (ii) the first variable domain is a VH domain and the first constant domain is a CL domain, and optionally the polypeptide is associated with a second polypeptide, wherein the second polypeptide comprises an antibody CH1 constant domain that is paired with the CL domain; (iii) the first variable domain is a VL domain and the first constant domain is a CH1 domain, and optionally the polypeptide is associated with a second polypeptide, wherein the second polypeptide comprises an antibody CL constant domain that is paired with the CH1 domain; or (iv) the first variable domain is a VL domain and the first constant domain is a CL domain, and optionally the polypeptide is associated with a second polypeptide, wherein the second polypeptide comprises an antibody CH1 constant domain that is paired with the CL domain.
26. The polypeptide of any one of Paragraphs 21 to 25, comprising an antibody Fc region or antibody single variable domain between (v) the first variable domain or scFv and (vi) the SAM, wherein the Fc comprises a CH2 and a CH3.
27. The polypeptide of any one of Paragraphs 21 to 26, comprising the Fc region or an antibody single variable domain between (i) the SAM and (ii) the most C-terminal variable domain.
28. The polypeptide of any one of Paragraphs 21 to 27, comprising in N- to C-terminal direction the antibody Fc region and the most N-terminal variable domain or scFv.
29. The polypeptide of any one of Paragraphs 21 to 28, comprising in N- to C-terminal direction the SAM and the antibody Fc region.
30. The polypeptide of any one of Paragraphs 21 to 29, wherein the first or each linker is a (G₄S)ₙ linker, wherein n=1, 2, 3, 4, 5, 6, 7, 8, 9 or 10.
31. The polypeptide of any preceding Paragraph, wherein each domain and SAM is a human domain and SAM respectively.
32. The polypeptide of any preceding Paragraph, wherein the polypeptide comprises binding specificity for more than one antigen, optionally 2, 3 or 4 different antigens.
33. A multimer (optionally a tetramer) of a polypeptide according to any preceding Paragraph.
34. A multimer of a plurality of antibody Fc regions, wherein each Fc is comprised by a respective polypeptide and is unpaired with another Fc region.
35. The multimer of Paragraph 34, wherein the multimer is a polypeptide tetramer comprising at least 4 Fc regions.
36. The multimer of Paragraph 34 or 35, wherein each Fc region is identical.
37. The multimer of any one of Paragraphs, wherein each Fc comprise a CH2 and a CH3, wherein each CH2 is a CH2 as recited in any one of Paragraphs 1 to 32.
38. The polypeptide or multimer of any preceding Paragraph, comprising eukaryotic cell glycosylation.
39. The polypeptide or multimer of Paragraph 38, wherein the cell is a HEK293, CHO or Cos cell.
40. The polypeptide or multimer of any preceding Paragraph for medical use.
41. A pharmaceutical composition comprising the polypeptide or multimer of any preceding Paragraph.
42. A nucleic acid encoding a polypeptide of any one of Paragraphs 1 to 32 and 38 to 40.
43. A eukaryotic cell or vector comprising the nucleic acid of Paragraph 42.
44. A method of binding multiple copies of an antigen, the method comprising combining the copies with a multimer of any one of Paragraphs 33 to 40, wherein the copies are bound by polypeptides of the multimer, and optionally the method comprising isolating the multimer bound to the antigen copies.
45. The method of Paragraph 37 wherein the method is a diagnostic method for detecting the presence of a substance in a sample, wherein the substance comprises the antigen, the method comprising providing the sample (eg, a bodily fluid, food, food ingredient, beverage, beverage ingredient, soil or forensic sample), mixing the sample with multimers according to any one of Paragraphs 33 to 40 and detecting the binding of multimers to the antigen in the sample.
46. A method of treating or reducing the risk of a disease or condition in a human or animal subject, the method comprising administering the composition of Paragraph 41 to the subject, wherein multimers comprised by the composition specifically bind to a target antigen in the subject, wherein said binding mediates the treatment or reduction in risk.
47. The method of Paragraph 46, wherein the antigen is an immune checkpoint antigen (eg, PD-L1, PD-1 or CTLA4); or wherein the antigen is TNF alpha or IL-17A.
48. The method of Paragraph 46, wherein the antigen mediates the disease or condition in the subject; and optionally wherein the binding antagonises the antigen.
49. A composition comprising a plurality of polypeptides according to any one of Paragraphs 1 to 32 and 38 to 40, wherein at least 90% of the polypeptides are comprised by tetramers of said polypeptides.
50. The composition of Paragraph 49, wherein at least 98% of the polypeptides are comprised by tetramers of said polypeptides.
51. The composition of Paragraph 49 or 50, wherein the remaining polypeptides are selected from one or more of polypeptide monomers, dimers and trimers.
52. A method of producing a composition (optionally a composition according to any one of Paragraphs 49 to 51) comprising a plurality of polypeptides according to any one of Paragraphs 1 to 32 and 38 to 40, the method comprising providing eukaryotic host cells according to Paragraph 34, culturing the host cells, and allowing expression and secretion from the cells of tetramers of the polypeptides, and optionally isolating or purifying the tetramers.

### Concepts

In certain embodiments, the invention is useful for providing multimers for treating cancer in humans or animals. In this respect, it may be useful to use the multimers to target tumours by binding to tumour-associated antigen and/or to bind to T-cells to modulate their activity. For example the multimers may bind to an antigen on T regulatory cells (Tregs) to downregulate their activity. Additionally or alternatively, the multimers may bind to T effector (Teff) cells to upregulate their activity. The provision of an antibody Fc region in the polypeptides of multimers may be advantageous for providing Fc effector functions and/or cytotoxicity for killing tumour cells. In one advantageous configuration, the invention exploits the ability to provide multiple identical antigen or epitope binding sites that can be used to bind to several copies of the same antigen or epitope on tumour cells, thereby providing for an avidity affect wherein the multimers bind preferentially to tumour cells over any non-target or normal cells, since the former surface-express more copies of the antigen than normal cells. In one configuration, when the multimer also comprises binding sites for an immune checkpoint regulator. In one example the regulator is an immune checkpoint inhibitor and the binding sites antagonise the inhibitor. This is useful, for example when the inhibitor is expressed by Teff cells, for upregulating Teff activity in the vicinity of tumour cells that are targeted by the multimer (eg, by binding TAA on the tumour cells). In another example the regulator is an immune checkpoint stimulator and the binding sites agonise the inhibitor. This is useful, for example when the inhibitor is expressed by Teff cells, for upregulating Teff activity in the vicinity of tumour cells that are targeted by the multimer (eg, by binding TAA on the tumour cells). Thus, upregulation of T-cell activity may be stimulated in the vicinity of tumour cells, rather in the vicinity of non-target (eg, normal or non-cancerous) cells. To this end, the invention provides the following Concepts. The following Concepts are not to be interpreted as Claims. The Claims start after the Examples section.
1. A polypeptide comprising a self-associating multimerisation domain (SAM), a first antigen binding site and a second antigen binding site, wherein the first site specifically binds to a first antigen or epitope, and the second binding site specifically binds to a second antigen or epitope, wherein each antigen or epitope is a tumour-associated antigen (TAA) or epitope, or an immune checkpoint regulator (eg, inhibitor) antigen or epitope.
2. The polypeptide of Concept 1, wherein the first antigen is a TAA and the second antigen is an immune checkpoint regulator (eg, inhibitor).
3. The polypeptide of Concept 1, wherein the first antigen is a TAA and the second antigen is a TAA.
4. The polypeptide of Concept 1, wherein the first antigen is an immune checkpoint inhibitor and the second antigen is an immune checkpoint regulator (eg, inhibitor).
5. The polypeptide of Concept 3 or 4, wherein the binding sites are capable of specifically binding to the same epitope of the same antigen.
6. The polypeptide of Concept 3 or 4, wherein the binding sites are capable of specifically binding to different epitopes of the same antigen.
7. The polypeptide of any preceding Concept, wherein the first antigen is selected from 4-1BB, 4-1BBL, CD28, OX40, OX40L, ICOS, ICOSL, GITR, CD40, CD27, CD27L, CD40L, LIGHT, CD70, CD80, CD86, HER2, HER3, PSMA, WT1, MUC1, LMP2, EGFRvIII, MAGE A3, GD2, CEA, Melan a/MART1, Bcr-Abl, Survivin, PSA, hTERT, EphA2, PAP, EpCAM, ERG, PAX3, ALK, Androgen receptor, Cyclin B1, RhoC, GD3, PSCA, PAX5, LCK, VEGFR2, MAD CT-1, FAP, MAD CT-2, PDGFR-beta, Fos related antigen 1, NY-BR-1, ETV6-AML, RGS5, SART3, SSX2, XAGE-1, STn, PAP and BCMA.
8. The polypeptide of any preceding Concept, wherein the second antigen is selected from PDL1, PD1, CTLA4, BTLA, KIR, LAG3, TIM3, A2aR, HVEM, GAL9, VISTA, SIRPa, CD47, CD160, CD155, IDO, CEACAM1, 2B4, CD48 and TIGIT.
9. The polypeptide of any preceding Concept, wherein the polypeptide comprises a third antigen binding site that is capable of specifically binding to a third antigen or epitope.
10. The polypeptide of Concept 9, wherein the third antigen is a TAA.
11. The polypeptide of Concept 9, wherein the third antigen is an immune checkpoint regulator (eg, inhibitor).
12. The polypeptide of any one of Concepts 9 to 11, wherein the third antigen is the same as the first or second antigen.
13. The polypeptide of any one of Concepts 9 to 12, wherein the third antigen is CD3 (eg, CD3e) or CD28.
14. The polypeptide of any one of Concepts 9 to 13, wherein the polypeptide comprises a fourth antigen binding site that is capable of specifically binding to a fourth antigen or epitope.
15. The polypeptide of Concept 14, wherein the fourth antigen is a TAA.
16. The polypeptide of Concept 14, wherein the fourth antigen is an immune checkpoint regulator (eg, inhibitor).
17. The polypeptide of any preceding Concept, wherein the polypeptide is according to any polypeptide disclosed herein.

In an example, said first dAb or first scFv of the polypeptide herein is the first antigen binding site of these Concepts; and optionally when a further dAb or scFv binding site is present this is the second antigen binding site of the Concepts.

In an example, said first dAb or first scFv of the polypeptide herein is the second antigen binding site of these Concepts; and optionally when a further dAb or scFv binding site is present this is the first antigen binding site of the Concepts.
18. A multimer of a polypeptide of any preceding Concept.
19. The multimer of Concept 18 for administration to a human or animal subject for targeting of an immune checkpoint inhibitor and an immune co-stimulatory molecule for the treatment of cancer.
20. A method of treating a cancer in a human or animal subject, the method comprising administering the multimer of claim 18 to the subject.

### Clauses

In a configuration, the invention provides the following Clauses. The following Clauses are not to be interpreted as Claims. The Claims start after the Examples section.
1. A protein multimer of at least first, second, third and fourth copies of an effector domain (eg, a protein domain) or a peptide, wherein the multimer is multimerised by first, second, third and fourth self-associating tetramerisation domains (TDs) which are associated together, wherein each tetramerisation domain is comprised by a respective engineered polypeptide comprising one or morecopies of said protein domain or peptide.
2. The multimer of Clause 1, wherein the multimer is a tetramer, octamer, dodecamer, hexadecamer or 20-mer (eg, tetramer or an octamer) of said domain or peptide.
3. The multimer of any Clause 1 or 2, comprising a tetramer, octamer, dodecamer, hexadecamer or 20-mer (eg, tetramer or an octamer) of an immunoglobulin superfamily binding site.
4. The multimer of Clause 3, wherein the binding site comprises a first variable domain paired with a second variable domain.
5. The multimer of any preceding Clause, wherein each polypeptide comprises first and second copies of said protein domain or peptide, wherein the polypeptide comprises in (N- to C-terminal direction) (i) a first of said copies - TD - the second of said copies; (ii) TD - and the first and second copies; or (iii) said first and second copies - TD.
6. The multimer of any preceding Clause, wherein the TDs are NHR2 TDs and the domain or peptide is not a NHR2 domain or peptide; or wherein the TDs are p53 TDs and the domain or peptide is not a p53 domain or peptide.
7. The multimer of any preceding Clause, wherein the engineered polypeptide comprises one or more copies of a second type of protein domain or peptide, wherein the second type of protein domain or peptide is different from the first protein domain or peptide.
8. The multimer of any preceding Clause, wherein the domains are immunoglobulin superfamily domains.
9. The multimer of any preceding Clause, wherein the domain or peptide is an antibody variable or constant domain, a TCR variable or constant domain, an incretin, an insulin peptide, or a hormone peptide.
10. The multimer of any preceding Clause, wherein the multimer comprises first, second, third and fourth identical copies of a said engineered polypeptide, the polypeptide comprising a TD and one (but no more than one), two (but no more than two) or more copies of the said protein domain or peptide.
11. The multimer of any preceding Clause, wherein the engineered polypeptide comprises an antibody or TCR variable domain (V1) and a NHR2 TD.
12. The multimer of Clause 11, wherein the polypeptide comprises (in N- to C-terminal direction) (i) V1-an optional linker-NHR2 TD; (ii) V1-an optional linker-NHR2 TD-optional linker-V2; or (iii) V1-an optional linker-V2 - optional linker - NHR2 TD, wherein V1 and V2 are TCR variable domains and are the same or different, or wherein V1 and V2 are antibody variable domains and are the same or different.
13. The multimer of Clause 12, wherein V1 and V2 are antibody single variable domains.
14. The multimer of Clause 11, wherein each engineered polypeptide comprises (in N- to C-terminal direction) V1-an optional linker- TD, wherein V1 is an antibody or TCR variable domain and each engineered polypeptide is paired with a respective second engineered polypeptide that comprises V2, wherein V2 is a an antibody or TCR variable domain respectively that pairs with V1 to form an antigen or pMHC binding site, and optionally one polypeptide comprises an antibody Fc, or comprises antibody CH1 and the other polypeptide comprises an antibody CL that pairs with the CH1.
15. The multimer of any preceding Clause, wherein the TD comprises (i) an amino acid sequence identical to SEQ ID NO: 10 or 126 or at least 80% identical thereto; or (ii) an amino acid sequence identical to SEQ ID NO: 120 or 123 or at least 80% identical thereto.
16. The multimer of any preceding Clause, wherein the multimer comprises a tetramer, octamer, dodecamer, hexadecamer or 20-mer (eg, tetramer or an octamer) of an antigen binding site of an antibody selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Keytruda™, Opdivo™, Gazyva™ and Obinutuzumab.
17. An isolated tetramer, octamer, dodecamer, hexadecamer or 20-mer of a TCR binding site, insulin peptide, incretin peptide or peptide hormone; or a plurality of said tetramers or octamers.
18. The multimer, tetramer, octamer, dodecamer, hexadecamer or 20-mer (eg, tetramer or an octamer) of any preceding Clause, wherein the mulitmer, tetramer, octamer, dodecamer, hexadecamer or 20-mer is
   (a) soluble in aqueous solution;
   (b) secretable from a eukaryotic cell; and/or
   (c) an expression product of a eukaryotic cell.
19. A tetramer, octamer, dodecamer, hexadecamer or 20-mer (eg, tetramer or an octamer) of
   (a) TCR V domains or TCR binding sites, wherein the tetramer, octamer, dodecamer, hexadecamer or 20-mer is soluble in aqueous solution;
   (b) antibody single variable domains, wherein the tetramer, octamer, dodecamer, hexadecamer or 20-mer is soluble in aqueous solution;
   (c) TCR V domains or TCR binding sites, wherein the tetramer, octamer, dodecamer, hexadecamer or 20-mer is capable of being intracellularly and/or extracellularly expressed by HEK293 cells; or
   (d) antibody variable domains, wherein the tetramer, octamer, dodecamer, hexadecamer or 20-mer is capable of being intracellularly and/or extracellularly expressed by HEK293 cells.
20. The multimer, tetramer, octamer, dodecamer, hexadecamer or 20-mer of any preceding Clause, wherein the tetramer, octamer, dodecamer, hexadecamer or 20-mer is bi-specific for antigen or pMHC binding.
21. The multimer, tetramer, octamer, dodecamer, hexadecamer or 20-mer of any preceding Clause, wherein the domains are identical.
22. The multimer, tetramer, octamer, dodecamer, hexadecamer or 20-mer of any preceding Clause, wherein the multimer, tetramer, octamer, dodecamer, hexadecamer or 20-mer comprises eukaryotic cell glycosylation.
23. The multimer, tetramer, octamer, dodecamer, hexadecamer or 20-mer of Clause 22, wherein the cell is a HEK293 cell.
24. A plurality of multimers, tetramers, octamers, dodecamesr, hexadecamers or 20-mers (eg, tetramers or octamers) of any preceding Clause.
25. A pharmaceutical composition comprising the multimer(s), tetramer(s), octamer(s), dodecamer(s), hexadecamer(s) or 20-mer(s) (eg, tetramer(s) or octamer(s)) of any preceding Clause and a pharmaceutically acceptable carrier, diluent or excipient.
26. A cosmetic, foodstuff, beverage, cleaning product, detergent comprising the multimer(s), tetramer(s), octamer(s), dodecamer(s), hexadecamer(s) or 20-mer(s) (eg, tetramer(s) or octamer(s)) of any one of Clauses 1 to 24.
27. A said engineered (and optionally isolated) polypeptide or a monomer (optionally isolated) of a multimer, tetramer, octamer, dodecamer, hexadecamer or 20-mer of any preceding Clause.
28. An engineered (and optionally isolated) polypeptide (P1) which comprises (in N- to C-terminal direction):-
   (a) TCR V1 -TCR C1 - antibody CH1 - optional linker - TD, wherein
      (i) V1 is a Vα and C1 is a Cα;
      (ii) V1 is a Vβ and C1 is a Cβ;
      (iii) V1 is a Vγ and C1 is a Cγ; or
      (iv) V1 is a Vδ and C1 is a Cδ;
      or
   (b) TCR V1 - antibody CH1 optional linker - TD, wherein
      (i) V1 is a Vα;
      (ii) V1 is a Vβ;
      (iii) V1 is a Vγ; or
      (iv) V1 is a Vδ;
      or
   (c) antibody V1 - antibody CH1 optional linker - TD, wherein
      (i) V1 is a VH; or
      (ii) V1 is a VL;
      or
   (d) antibody V1 - optional antibody CH1 antibody Fc - optional linker - TD, wherein
      (i) V1 is a VH; or
      (ii) V1 is a VL;
      or
   (e) antibody V1 - antibody CL - optional linker - TD, wherein
      (i) V1 is a VH; or
      (ii) V1 is a VL;
      or
   (f) TCR V1 -TCR C1 - optional linker - TD, wherein
      (i) V1 is a Vα and C1 is a Cα;
      (ii) V1 is a Vβ and C1 is a Cβ;
      (iii) V1 is a Vγ and C1 is a Cγ; or
      (iv) V1 is a Vδ and C1 is a Cδ.
29. The polypeptide of Clause 28, wherein the engineered polypeptide P1 is paired with a further polypeptide (P2), wherein P2 comprises (in N- to C-terminal direction):-
   (g) TCR V2 -TCR C2 - antibody CL, wherein P1 is according to (a) recited in Clause 28 and
      (i) V2 is a Vα and C2 is a Cα when P1 is according to (a)(ii);
      (ii) V2 is a Vβ and C2 is a Cβ when P1 is according to (a)(i);
      (iii) V2 is a Vγ and C2 is a Cγ when P1 is according to (a)(iv); or
      (iv) V2 is a Vδ and C2 is a Cδ when P1 is according to (a)(iii);
      or
   (h) TCR V2 - antibody CL, wherein P1 is according to (b) recited in Clause 28 and
      (i) V2 is a Vα when P1 is according to (b)(ii);
      (ii) V2 is a Vβ when P1 is according to (b)(i);
      (iii) V2 is a Vγ when P1 is according to (b)(iv); or
      (iv) V2 is a Vδ when P1 is according to (b)(iii);
      or
   (i) Antibody V2 - CL, wherein P1 is according to (c) recited in Clause 28 and
      (i) V2 is a VH when P1 is according to (c)(ii); or
      (ii) V2 is a VL when P1 is according to (c)(i);
      or
   (j) Antibody V2 - optional CL, wherein P1 is according to (d) recited in Clause 28 and
      (i) V2 is a VH when P1 is according to (d)(ii); or
      (ii) V2 is a VL when P1 is according to (d)(i);
      or
   (k) Antibody V2 - CH1, wherein P1 is according to (e) recited in Clause 28 and
      (i) V2 is a VH when P1 is according to (e)(ii); or
      (ii) V2 is a VL when P1 is according to (e)(i);
      or
   (1) TCR V2 -TCR C2, wherein Pis according to (f) recited in Clause 28 and
      (i) V2 is a Vα and C2 is a Cα when P1 is according to (f)(ii);
      (ii) V2 is a Vβ and C2 is a Cβ when P1 is according to (f)(i);
      (iii) V2 is a Vγ and C2 is a Cγ when P1 is according to (f)(iii); or
      (iv) V2 is a Vδ and C2 is a Cδ when P1 is according to (f)(iv).
30. A multimer of P1 as defined in Clause 28; or of P1 paired with P2 as defined in Clause 29; or a plurality of said multimers, optionally wherein the multimer is according to any one of Clauses 1 to 24.
31. A nucleic acid encoding an engineered polypeptide or monomer of any one of Clauses 27 to 29, optionally wherein the nucleic acid is comprised by an expression vector for expressing the polypeptide.
32. A eukaryotic host cell comprising the nucleic acid or vector of Clause 31 for intracellular and/or secreted expression of the multimer, tetramer, octamer, dodecamer, hexadecamer or 20-mer (eg, tetramer, octamer), engineered polypeptide or monomer of any one of Clauses 1 to 24.
33. Use of a nucleic acid or vector according to Clause 31 in a method of manufacture of protein multimers for producing intracellularly expressed and/or secreted multimers, wherein the method comprises expressing the multimers in and/or secreting the multimers from eukaryotic cells comprising the nucleic acid or vector.
34. Use of a nucleic acid or vector according to Clause 31 in a method of manufacture of protein multimers for producing glycosylated multimers in eukaryotic cells comprising the nucleic acid or vector.
35. A mixture comprising (i) a eukaryotic cell line encoding an engineered polypeptide according to any one of Clauses 27 to 29; and (ii) multimers, tetramers, octamers, dodecamers, hexadecamers or 20-mers (eg, tetramers or octamers)as defined in any one of Clauses 1 to 24.
36. The mixture of Clause 35, wherein the cell line is in a medium comprising secretion products of the cells, wherein the secretion products comprise said multimers, tetramers, octamers, dodecamers, hexadecamers or 20-mers (eg, tetramers or octamers).
37. The multimer, tetramer, octamer, dodecamer, hexadecamer or 20-mer (eg, tetramer, octamer) of any one of Clauses 1 to 24 for medical use.
38. A method producing
   (a) TCR V domain multimers, the method comprising the soluble and/or intracellular expression of TCR V-NHR2 TD or TCR V- p53 TD fusion proteins expressed in eukaryotic cells, the method optionally comprising isolating a plurality of said multimers;
   (b) antibody V domain multimers, the method comprising the soluble and/or intracellular expression of antibody V -NHR2 TD or V- p53 TD fusion proteins expressed in eukaryotic cells, the method optionally comprising isolating a plurality of said multimers;
   (c) incretin peptide multimers, the method comprising the soluble and/or intracellular expression of incretin peptide-NHR2 TD or incretin peptide-p53 TD fusion proteins expressed in eukaryotic cells, such as HEK293T cells; the method optionally comprising isolating a plurality of said multimers; or
   (d) peptide hormone multimers, the method comprising the soluble and/or intracellular expression of peptide hormone-NHR2 TD or peptide hormone- p53 TD fusion proteins expressed in eukaryotic cells, such as HEK293T cells; the method optionally comprising isolating a plurality of said multimers.
39. Use of self-associating tetramerisation domains (TD) in a method of the manufacture of a tetramer of polypeptides, for producing a higher yield of tetramers versus monomer and/or dimer polypeptides.
40. Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerisation domains (TD).
41. Use of self-associating tetramerisation domains (TD) in a method of the manufacture of a tetramer of a polypeptide, for producing a plurality of tetramers that are not in mixture with monomers, dimers or trimers.
42. Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerisation domains (TD).
43. The use of any one of Clauses 39 to 42, wherein the yield of tetramers is at least 10x the yield of monomers and/or dimers.
44. The use of any one of Clauses 39 to 43, wherein the ratio of tetramers produced : monomers and/or dimers produced in the method is at least 90:10.
45. The use of any one of Clauses 39 to 44, wherein each monomer has a size of no more than 40 kDa.
46. The use of any one of Clauses 39 to 45, wherein each tetramer has a size of no more than 150 kDa.
47. The use of any one of Clauses 39 to 46, wherein the method comprises expressing the tetramers from a eukaryotic cell line.
48. A multivalent heterodimeric soluble T cell receptor capable of binding pMHC complex comprising:
   (a) TCR extracellular domains;
   (b) immunoglobulin constant domains; and
   (c) an NHR2 multimerisation domain of ETO.
49. A multimeric immunoglobulin, comprising
   (i) immunoglobulin variable domains; and
   (ii) an NHR2 multimerisation domain of ETO.
50. A method for assembling a soluble, multimeric polypeptide, comprising:
   (a) providing a monomer of the said multimeric polypeptide, fused to an NHR2 domain of ETO; and
   (b) causing multiple copies of said monomer to associate, thereby obtaining a multimeric, soluble polypeptide.

In any disclosure herein, the or each constant region or domain, the CH2, the CH3, the CH2 and CH3 or the Fc is respectively a constant region or domain, the CH2, the CH3, the CH2 and CH3 or the Fc of a human constant region. For example, the constant region is selected from the group IGHA1*01, IGHA1*02, IGHA1*03, IGHA2*01, IGHA2*02, IGHA2*03, IGHD*01, IGHD*02, IGHE*01, IGHE*02, IGHE*03, IGHE*04, IGHEP1*01, IGHEP1*02, IGHEP1*03, IGHEP1*04, IGHG1*01, IGHG1*02, IGHG1*03, IGHG1*04, IGHG1*05, IGHG1*06, IGHG1*07, IGHG1*08, IGHG1*09, IGHG1*10, IGHG1*11, IGHG1*12, IGHG1*13, IGHG1*14, IGHG2*01, IGHG2*02, IGHG2*03, IGHG2*04, IGHG2*05, IGHG2*06, IGHG2*07, IGHG2*08, IGHG2*09, IGHG2* 10, IGHG2* 11, IGHG2* 12, IGHG2* 13, IGHG2* 14, IGHG2* 15, IGHG2* 16, IGHG2* 17, IGHG3*01, IGHG3*02, IGHG3*03, IGHG3*04, IGHG3*05, IGHG3*06, IGHG3*07, IGHG3*08, IGHG3*09, IGHG3* 10, IGHG3* 11, IGHG3* 12, IGHG3* 13, IGHG3* 14, IGHG3* 15, IGHG3* 16, IGHG3 * 17, IGHG3* 18, IGHG3* 19, IGHG3*20, IGHG3*21, IGHG3*22, IGHG3*23, IGHG3*24, IGHG3*25, IGHG3*26, IGHG3*27, IGHG3*28, IGHG3*29, IGHG4*01, IGHG4*02, IGHG4*03, IGHG4*04, IGHG4*05, IGHG4*06, IGHG4*07, IGHG4*08, IGHGP*01, IGHGP*02, IGHGP*03, IGHM*01, IGHM*02, IGHM*03 and IGHM*04 (eg, the constant region is a *01 allele listed in said group, preferably the constant region is a human IGHG1*01 or IGHM*01 constant region). In an alternative, the constant region is a non-human (eg, mammal, rodent, mouse, rat, dog, cat or horse) constant region, such as a homologue of a human constant region listed in said group.

The polypeptide, in one embodiment, comprises (in N- to C-terminal direction) a first antigen binding site (eg, a dAb), an antibody CH1 (eg, human IgGl CH1), a hinge sequence comprising a lower hinge and devoid of a core hinge region (and optionally devoid of an upper hinge region), an antibody Fc region and a SAM (eg, a TD, such as a p53 TD). For example, the core hinge region sequence is a CXXC amino acid sequence. The polypeptide may comprise another antigen binding site (eg a dAb or scFv) between the first binding site and the CH1, between the Fc and SAM and/or C-terminal to the SAM. Optionally, the multimer comprises a plurality (eg, 4 copies) of such polypeptide, for example wherein each polypeptide is paired with a further polypeptide comprising (in N- to C-terminal direction) a second antigen binding site (eg, a dAb), an antibody CL (eg, a human Cκ) and optionally a third antigen binding site. Optionally the binding sites have the same antigen specificity (eg, all bind TNF alpha). In another option, the first and second (and optionally said another binding site) bind to different antigens. The or each binding site can bind any antigen disclosed herein, eg, each binding site binds TNF alpha (as shown in Example 17). In another example, the first antigen binding site is a VH of an antigen binding site of a predetermined antibody that specifically binds to the antigen (and the CH1 is optionally the CH1 of the antibody), and the second binding site of the further polypeptide is a VL of the antigen binding site of the predetermined antibody (and the CL is optionally the CL of the antibody), wherein the VH and VL pair to form a VH/VL binding site which has binding specificity for the antigen. The predetermined antibody may be a marketed antibody, for example, as shown in Example 19. For example, the VH/VL binding site specifically binds to CTLA-4, eg, wherein the predetermined antibody is ipilimumab (or Yervoy™). For example, the VH/VL binding site specifically binds to TNF alpha, eg, wherein the predetermined antibody is adalimumab, golimumab, infliximab (or Humira™, Simponi™ or Remicade™). For example, the VH/VL binding site specifically binds to PD-L1, eg, wherein the predetermined antibody is avelumab (or Bavencio™) or atezolizumab (or Tecentriq™). For example, the VH/VL binding site specifically binds to PD-1, eg, wherein the predetermined antibody is nivolumab (or Opdivo™) or pembrolizumab (or Keytruda™). For example, the VH/VL binding site specifically binds to VEGF, eg, wherein the predetermined antibody is bevacizumab (or Avastin™) or ranibizumab (or Lucentis™). In another example, the polypeptide comprises (in N- to C-terminal direction) a first VEGF binding site, an optional second VEGF binding site, an antibody CH1 (eg, human IgGl CH1), a hinge sequence comprising a lower hinge and devoid of a core hinge region (and optionally devoid of an upper hinge region), an antibody Fc region and a SAM (eg, a TD, such as a p53 TD). In an example, the first binding site is a Ig domain 2 from VEGFR1 and the second binding site is Ig domain 3 from VEGFR2 (as shown in Example 20). In another example, the first binding site is a Ig domain 3 from VEGFR2 and the second binding site is Ig domain 2 from VEGFR2. In an example, the first and second binding domains are (in N- to C-terminal direction) the first and second VEGF binding sites of aflibercept (or Eylea™).

Suitable predetermined antibodies are ReoPro™; Abciximab; Rituxanh™; Rituximab; Zenapaxh™; Daclizumab; Simulecth™; Basiliximab; Synagis™; Palivizumab; Remicadeh™; Infliximab; Herceptinh™; Trastuzumab; Mylotargh™; Gemtuzumab; Campathh™; Alemtuzumab; Zevalinh™; Ibritumomab; Humirah™; Adalimumab; Xolair™; Omalizumab; Bexxarh™; Tositumomab; Raptivah™; Efalizumab; Erbituxh™; Cetuximab; Avastinh™; Bevacizumab; Tysabrih™; Natalizumab; Actemrah™; Tocilizumab; Vectibixh™; Panitumumab; Lucentish™; Ranibizumab; Solirish™; Eculizumab; Cimziah™; Certolizumab; Simponih™; Golimumab, Ilaris™; Canakinumab; Stelara™; Ustekinumab; Arzerrah™; Ofatumumab; Prolie™; Denosumab; Numaxh™; Motavizumab; ABThraxh™; Raxibacumab; Benlystah™; Belimumab; Yervoyh™; Ipilimumab; Adcetrish™; Brentuximab; Vedotin™; Perjeta™; Pertuzumab; Kadcyla™; Ado-trastuzumab; Gazyva™ and Obinutuzumab. Also disclosed are the generic versions of these and the corresponding INN names - each of which is a suitable predetermined antibody for use as a source of antigen binding sites for use in the present invention. Suitable sequences of VH and VL domains of predetermined antibodies are disclosed in Table 4. Thus, for example, the multimer of the invention comprises a plurality (eg, 4, 8, 12, 16 or 20) copies of the VH/VL antigen binding site of any of these antibodies, eg, wherein the VH of the binding site is comprised by a polypeptide of the invention that comprises a SAM (eg, a TD) and each polypeptide is paired with a further polypeptide comprising the VL that pairs with the VH, thus forming an antigen binding site. In an example, the polypeptide comprising the SAM also comprises a CH1 which pairs with a CL of the further polypeptide. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

In one embodiment, the predetermined antibody is Avastin.

In one embodiment, the predetermined antibody is Actemra.

In one embodiment, the predetermined antibody is Erbitux.

In one embodiment, the predetermined antibody is Lucentis.

In one embodiment, the predetermined antibody is sarilumab.

In one embodiment, the predetermined antibody is dupilumab.

In one embodiment, the predetermined antibody is alirocumab.

In one embodiment, the predetermined antibody is evolocumab.

In one embodiment, the predetermined antibody is pembrolizumab.

In one embodiment, the predetermined antibody is nivolumab.

In one embodiment, the predetermined antibody is ipilimumab.

In one embodiment, the predetermined antibody is remicade.

In one embodiment, the predetermined antibody is golimumab.

In one embodiment, the predetermined antibody is ofatumumab.

In one embodiment, the predetermined antibody is Benlysta.

In one embodiment, the predetermined antibody is Campath.

In one embodiment, the predetermined antibody is rituximab.

In one embodiment, the predetermined antibody is Herceptin.

In one embodiment, the predetermined antibody is durvalumab.

In one embodiment, the predetermined antibody is daratumumab.

In another embodiment, the polypeptide comprises (in N- to C-terminal direction) a first antigen binding site, an optional linker (eg, a G₄Sₙ, wherein n=1, 2, 3, 4, 5, 6, 7, ot 8, preferably 3), a second antigen binding site, a hinge sequence comprising a lower hinge and devoid of a core hinge region (and optionally devoid of an upper hinge region), an antibody Fc (eg, an IgGl Fc) and a SAM (eg, a TD, such as a p53 TD). For example, the core hinge region sequence is a CXXC amino acid sequence. The polypeptide may comprise another antigen binding site (eg a dAb or scFv) between the Fc and SAM and/or C-terminal to the SAM. Optionally, the multimer comprises a plurality (eg, 4 copies) of such polypeptide. Optionally the binding sites have the same antigen specificity (eg, all bind TNF alpha). In another option, the first and second (and optionally said another binding site) bind to different antigens. The or each binding site can bind any antigen disclosed herein, eg, each binding site binds PD-L1, or the first binding site binds PD-L1 and the second binding site binds 41-BB, or the first binding site binds 4-1BB and the second binding site binds PD-L1 (as shown in Example 18).

The polypeptide, in one embodiment, comprises (in N- to C-terminal direction) a first antigen binding site (eg, a dAb), an optional linker (eg, a G₄Sₙ, wherein n=1, 2, 3, 4, 5, 6, 7, ot 8, preferably 3), a second antigen binding site (eg, a dAb), an antibody CH1 (eg, human IgGl CH1) and a SAM (eg, a TD, such as a p53 TD). The polypeptide may comprise another antigen binding site (eg a dAb or scFv) C-terminal to the SAM. Optionally, the multimer comprises a plurality (eg, 4 copies) of such polypeptide, for example wherein each polypeptide is paired with a further polypeptide comprising (in N- to C-terminal direction) a third antigen binding site (eg, a dAb), an optionaly fourth antigen binding site (eg, a dAb), an antibody CL (eg, a human Cκ or Cλ) and optionally a furhter antigen binding site. For example, the fourth and further binding sites are omitted. In another example, the third and fourth binding sites, but not the further binding site, are present. In another example, the third and further (but not the fourth) binding sites are present. Optionally the binding sites have the same antigen specificity (eg, all bind TNF alpha). In another option, the first and second (and optionally said another said binding site) bind to different antigens. The or each binding site can bind any antigen disclosed herein, eg, each binding site binds TNF alpha (as shown in Examples 21 and 22). In an example, the first and third, or the second and third binding sites pair to form a VH/VL pair that is identical to the VH/VL binding site of an anti-TNF alpha antibody, such as adalimumab, golimumab, infliximab (or Humira™, Simponi™ or Remicade™). In an example, the first and third, or the second and third binding sites pair to form a VH/VL pair that is identical to the VH/VL binding site of an anti-PD-L1 antibody, such as avelumab (or Bavencio™) or atezolizumab (or Tecentriq™). In an example, the first and third, or the second and third binding sites pair to form a VH/VL pair that is identical to the VH/VL binding site of an anti-PD-1 antibody, such as nivolumab (or Opdivo™) or pembrolizumab (or Keytruda™). In an example, the first and third, or the second and third binding sites pair to form a VH/VL pair that is identical to the VH/VL binding site of an anti-VEGF antibody, such as bevacizumab (or Avastin™) or ranibizumab (or Lucentis™). Predetermined antibodies as discussed above can be used as the source of the VH/VL pairs.

In an example, the polypeptide of the invention is any Quad polypeptide disclosed herein, eg, comprising the Quad amino acid shown in any of the Tables herein (eg, any one of SEQ ID Nos: 81-115, 151-162, 190, 191, 209-224 and 179) or encoded by any of the Quad nucleotide sequences in any of the Tables herein (eg, Table 9, 14 or 17), or having the structure of a polypeptide shown in Table 8. The SAM may be any SAM disclosed herein, eg, any p53 or homologue TD disclosed in any Table herein (eg, as shown in Table 7 or comprised by a protein in Table 13).

Where amino acid sequences are shown with plural histidines at their C-terminus (eg, “HHHHHH” optionally followed by “..AAA”), such histidines and the optional ..AAA are in one embodiment omitted and the corresponding nucleotides encoding this are omitted from the nucleic acid encoding the amino acid sequence. Where amino acid sequences are shown with a DYKDDDDK motif (eg, a DYKDDDDKHHHHHH or DYKDDDDKHHHHHH..AAA), such a motif is in one embodiment omitted and the corresponding nucleotides encoding this are omitted from the nucleic acid encoding the amino acid sequence.

### Further Configurations

As discussed herein, the invention provides configurations in which the polypeptide a self-associating multimerisation domain (SAM, eg, a TD) and a peptide, domain or an epitope or antigen binding site (eg, a dAb or an antibody variable domain). In an embodiment, the SAM is a TD, such a p53 TD as disclosed herein.

### Anti-Virus Examples, Surprising Expansion of Antigen Specificity Etc

In an example, the polypeptide comprises (eg, in N- to C-terminal direction) at least an extracellular domain (ECD) of a cell-surface protein that is a receptor for a virus or required for virus activation. For example, the protein poteolytically cleaves and activates a spike glycoprotein of the virus (eg, Coronoavirus or any other virus disclosed herein, such as in Table 19). Optionally, the entire cell-surface portion of the receptor is comprised by the polypeptide of the invention. In an example, the virus is capable of infecting human cells and the receptor is a cell-surface protein found on human cells (such as lung cells). In an example, the virus is capable of infecting non-human animal cells and the receptor is a cell-surface protein found on cells of such animal (such as lung cells). In an example, the virus is capable of infecting plant cells and the receptor is a cell-surface protein found on cells of such plant (such as a crop, wheat, corn, barley, tobacco, grass, fruiting plant or tree). By forming multimers using copies of the polypeptide according to the invention, multimers of the invention comprising copies of all or portions of such cell-surface proteins may act as sinks for binding several virus particles per copy of multimer. Advantageously, this will prevent the bound viruses from infecting cells of the human, animal, plant or other subject or environment in which the cells are present. So, for example, the invention provides a method of treating a viral infection in a human or animal subject, the method comprising administering a composition comprising a plurality of the multimers to a human or animal subject (eg, intravenously or by inhalation), wherein the subject is suffering from a virus infection and copies of the multimer bind to copies of the virus, thereby reducing the severity of the infection and/or reducing progression of the infection and/or reducing one or more symptoms of the infection (such as a inflammatory response). In another example, the composition can be used prophylactically; thus the invention provides a method of preventing or reducing the risk of a viral infection or a symptom thereof in a human or animal subject, the method comprising administering a composition comprising a plurality of the multimers to a human or animal subject (eg, intravenously or by inhalation), wherein the subject is at risk of suffering from a virus infection, thereby preventing or reducing the risk of the viral infection and/or preventing or reducing one or more symptoms of the infection (such as a inflammatory response). In an example, the virus is a Coronavirus.

In an example, the virus is a virus selected from Table 19.

For example, the virus is a Coronavirus, a MERS-Cov, a SARS-Cov, SARS-Cov-1 or preferably SARS-Cov-2. In this example, the receptor may be ACE2. In an alternative, the cell-surface protein is a TMPRSS protein, preferably a TMPRSS2 protein. Optionally, the polypeptide of the invention in this example comprises an ACE2 extracellular domain and a TMPRSS protein extracellular domain, optionally wherein the domains are human domains and the polypeptides (or multimers according to the invention comprising copies of such a polypeptide) are for treating or preventing a Coronavirus infection in a human. In an embodiment in this example, the SAM is a TD, such a p53 TD as disclosed herein.

An example of a protein required for virus activation is TMPRSS2 protein, eg, human TMPRSS2 protein (UniProtKB - 015393 (TMPS2 _HUMAN), the sequence with of which with identifier 015393-1 is explicitly incorporated herein for use in the invention and possible inclusion in one or more claims herein). In an example, the polypeptide of the invention comprises amino acids of human TMPRSS2 protein from amino acid 106 to 492.

In an alternative, the virus is selected from Coronavirus 229E (HCoV-229E), Coronavirus EMC (HCoV-EMC), Sendai virus (SeV), human metapneumovirus (HMPV), human parainfluenza 1, 2, 3, 4a and 4b viruses (HPIV), and influenza A virus (eg, strains H1N1, H3N2 and H7N9).

In an embodiment, the polypeptide comprises an angiotensin converting enzyme 2 (ACE2) protein as disclosed in any of US9,561,263; US 8,586,319; or EP2089715, EP2047867, EP2108695, EP2543724, EP2155871, EP2274005, EP3375872, EP2222330, EP2943216, EP2332582 or any US counterpart patent application or patent of any of these that shares a common priority. The disclosures of such proteins and their sequences as disclosed in these European and US patents and applications are explicitly incorporated herein for possible use in the invention, such as in polypepides or multimers of the invention. For example, the peptide or domain of the polypeptide of the invention comprises an ECD of entire ACE2 protein as disclosed in any one of these European and US patents and applications. Each such protein and sequence is also individually and explicitly incorporated herein such that any one of such proteins or sequences can be included in any claim herein as a component of a polypeptide or multimer of the invention. Also explicitly incorporated herein are the uses and medical diseases and conditions disclosed in any of such European and US patents and applications and the polypeptide or multimer or method or use of the present invention may be for treating, preventing or reducing the risk of any of such diseases or conditions and may be included in any claim herein.

As discussed, a polypeptide of the invention may comprises amino acid sequence from an ACE2 protein. In an embodiment, there is provided a polypeptide comprising an amino acid sequence selected from SEQ ID NOs: 229-231. In an embodiment, there is provided a polypeptide comprising an amino acid sequence selected from SEQ ID NOs: 229-231 with the exception that the polypeptide comprises an alternative SAM other than the p53 TD disclosed in such sequence. For example, the SAM is a p63, p73 or homologue TD as disclosed herein. In an embodiment, there is provided a polypeptide comprising an ACE2 amino acid sequence as comprised by any one of SEQ ID NOs: 229-231. The invention also provides a tetramer of the invention comprising 4 copies of such a polypeptide, as well as a composition of the invention comprising such a tetramer. Such a multimer or composition may preferably be for use in a method of treating, preventing or reducing the risk of a viral infection (eg, a Coronavirus, or preferably SARS-Cov-2 infection), hypertension or a lung condition (eg, an acute lung injury or inflammation) in a human.

For example, a polypeptide of the invention (eg, for treating or preventing a viral infection, preferably a Coronavirus, a MERS-Cov, a SARS-Cov, SARS-Cov-1 or SARS-Cov-2 infection) comprises the amino acid sequence from amino acid 18 to 615; or from 18 to 656 of SEQ ID NO: 1 disclosed in US9,561,263, which sequences are explicitly incorporated herein by reference for use in the present invention and for possible inclusion in one or more claims herein. Optionally, the polypeptide comprises the amino acid sequence from amino acid 18 to 615; or from 18 to 656; or from 18 to 740 of SEQ ID NO: 1 disclosed in US9,561,263, but does not comprise any other amino acids from such SEQ ID NO: 1. Optionally, the polypeptide comprises the amino acid sequence of SEQ ID NO: 2 disclosed in EP2332582, or an amino acid sequence that is at least 80, 85, 90, 95, 96, 97, 98 or 99% homologous thereto, which sequences are explicitly and individually incorporated herein by reference for use in the invention and possible inclusion in one or more claims herein. Optionally, N-glycosylation sites of Asn53, Asn90, Asn103, Asn322, Asn432, Asn546 and Asn690 of SEQ ID NO: 1 are sialyzed, eg, as disclosed in US8,568,319, which disclosure of sialyzation is explicitly incorporated herein for use in the invention.

In an alternative to treating, preventing or reducing the risk of a viral infection as discussed above, the invention instead provides polypeptides, multimers, methods and uses for treating, preventing or reducing the risk of inflammation in the subject (eg, in a human suffering from lung inflammation or at risk of such).

In an alternative to treating, preventing or reducing the risk of a viral infection as discussed above, the invention instead provides polypeptides, multimers, methods and uses for treating, preventing or reducing the risk of hypertension, heart failure (eg, congestive heart failure or chronic heart failure or acute heart failure), myocardial infarction, atherosclerosis, renal failure or insufficiency, polycystic kidney disease (PKD), or a pulmonary disease. Examples are disclosed in EP2543724, the disclosure of which are explicitly incorporated herein for use in the invention.

In an alternative to treating, preventing or reducing the risk of a viral infection as discussed above, the invention instead provides polypeptides, multimers, methods and uses for treating, preventing or reducing the risk of an acute lung injury (ALI), eg, ARDS (Adult Respiratory Distress Syndrome), SARS (Severe Acute Respiratory Syndrome) or MERS (Middle East Respiratory Syndrome).

Optionally, the polypeptide of the invention comprises eukaryotic cell, mammalian or human cell glycosylation eg, CHO or HEK293 or Cos cell glycosylation.

Optionally, the invention provides a composition (eg, for medical use as described herein, such as for treating, preventing or reducing the risk of a viral infection) comprising a plurality of polypeptides of the invention, wherein less than 15, 10, 5, 4, 3, 2, or 1% of all the polypeptides are comprised by the group consisting of polypeptide monomers, dimers and trimers. Additionally or alternatively, at least 80, 85, 90, 95, 96, 97, 98 or 99% of all of the polypeptides are comprised by multimers comprising 4 copies of the SAM (eg, p53 TD).

In an alternative to treating, preventing or reducing the risk of a viral infection as discussed above, the invention instead provides polypeptides, multimers, methods and uses for treating, preventing or reducing the risk of hypertension in the subject (eg, in a human suffering from a lung or cardiovascular condition or at risk of such).

In a configuration, the polypeptide of the invention comprises one or more binding sites for an antigen comprised by the extracellular part of a cell-surface protein that is a receptor (eg, ACE2 or a homologue or orthologue) for a virus or a protein (eg, TMPRSS2 protein) required for virus activation. In an embodiment, 1, 2, 3, 4 or 5 such binding sites are comprised by the polypeptide or by a multimer of the invention comprising copies of the polypeptide. In an example, each binding site is comprised by a dAb, Fv or scFv. In an example, the multimer comprises a plurality (eg, 4 and no more and no less than 4) copies of a polypepide of the invention comprising a SAM (eg, a TD) and each polypeptide as paired with a respective copy of a further polypeptide, wherein each polypeptide pair comprises a VH/VL antigen binding site. In an example, the binding site specifically binds to a spike glycoprotein of the virus, eg, any virus disclosed herein, preferably a Coronavirus, more preferably SARS-Cov-2. In an example, the binding site binds to 2 or more different Coronaviruses, eg, SARS-Cov-1 and 2. Advantageously, multimers comprising 4 (and no more or less than 4) copies of a heavy chain polypeptide comprising an amino acid sequence selected from SEQ ID NOs: 225-227, wherein each copy is paired with a copy of a polypeptide comprising the amino acid of SEQ ID NO: 228 will bind to a virus, such as a Coronavirus (eg, SAR-Cov-1 and/or SARS-Cov-2) and preferably SAR-Cov-1 and SARS-Cov-2. In an example, the binding site is a VH/VL antigen binding site of a SAR-Cov antibody, such as antibody CR3022, CR3006, CR3013 or CR3014 disclosed in PLoS Med. 2006 Jul;3(7):e237; “Human monoclonal antibody combination against SARS coronavirus: synergy and coverage of escape mutants”, ter Meulen J et al and J Virol. 2005 Feb; 79(3): 1635-1644; doi: 10.1128/JVI.79.3.1635-1644.2005; PMCID: PMC544131; PMID: 15650189, “Molecular and Biological Characterization of Human Monoclonal Antibodies Binding to the Spike and Nucleocapsid Proteins of Severe Acute Respiratory Syndrome Coronavirus”, Edward N. van den Brink et al, the sequences of these antibodies and all anti-virus antibodies in these papers and individually of their VH and VL domains are incorporated herein by reference for use in the invention and possible inclusion in one or more claims herein. In an example, the polypeptide of the invention comprises a first SAR-Cov antigen binding site and a second SAR-Cov antigen binding site wherein the first site comprises a VH/VL binding site of CR3022 and the second site compriss a VH/VL binding site of an antibody selected from CR3006, CR3013 and CR3014 (eg, CR3022/3014; CR3022/3006; CR3022/3013 or CR3022/3014). In an example, the multimer of the invention comprises a first SAR-Cov antigen binding site and a second SAR-Cov antigen binding site wherein the first site comprises a VH/VL binding site of CR3022 and the second site compriss a VH/VL binding site of an antibody selected from CR3006, CR3013 and CR3014 (eg, CR3022/3014; CR3022/3006; CR3022/3013 or CR3022/3014).

In a configuration, the polypeptide of the invention comprises one or more binding sites for human TMPRSS2 protein, for example, the polypeptide comprises a binding site for TMPRSS2 protein as disclosed in US20190300625, eg, the VH/VL pair of any anti-TMPRSS2 antibody disclosed in US20190300625, eg wherein the binding site comprises SEQ ID NOs: 17 and 18 disclosed in US20190300625; all of these sequences and binding site disclosures are incorporated herein by reference for use in the present invention and for possible inclusion in one or more claims herein. In a configuration, the polypeptide of the invention comprises one or more binding sites for human IL-6R, for example, the polypeptide comprises the VH/VL pair of sarilumab. In a configuration, the polypeptide of the invention comprises one or more binding sites for human IL-4R, for example, the polypeptide comprises the VH/VL pair of dupilumab. In a configuration, the polypeptide of the invention comprises one or more binding sites for human OX40L or OX40, eg, the VH/VL pair of oxelumab.

In a configuration, the multimer of the invention comprises binding sites for human TMPRSS2 protein, for example, the multimer comprises a plurality of copies of a binding site for TMPRSS2 protein as disclosed in US20190300625, eg, the VH/VL pair of any anti-TMPRSS2 antibody disclosed in US20190300625, eg wherein the binding site comprises SEQ ID NOs: 17 and 18 disclosed in US20190300625; all of these sequences and binding site disclosures are incorporated herein by reference for use in the present invention and for possible inclusion in one or more claims herein. In a configuration, the multimer of the invention comprises a plurality of copies of a binding sites for human IL-6R, for example, the VH/VL pair of sarilumab. In a configuration, the multimer of the invention comprises a plurality of copies of a binding sites for human IL-4R, for example, the VH/VL pair of dupilumab. In a configuration, the multimer of the invention comprises binding sites for human OX40L or OX40, eg, a plurality of clpies of the VH/VL pair of oxelumab.

Thus, generally multimers of the invention may advantageously be cross-reactive to more than one antigen (ie, bind to more than one antigen, such as first and second antigens which are different from each other); or may be capable of binding to an antigen using binding sites of the multimer, wherein the binding site as a monomer or dimer is not capable of binding to the antigen. For example, the binding by the multimer and by the monomer or dimer form are tested under identical conditions (eg, of temperature, pH, time and antigen concentration). For example, the binding site as a monomer or dimer means that one or two copies (but no more than one or two respectively) of the binding site when comprised by a protein are not capable of binding to the antigen (first antigen). Thus, in that example the protein is monovalent or bivalent for the antigen. For example, the binding site is a VH/VL binding site of a 4-chain antibody having 2 copies (but no more than 2) of the antigen binding site, wherein the antibody is not capable of binding to the antigen (eg, TACI); optionally the antigen is an antigen that is cognate to a receptor or ligand (eg, APRIL when the antigen is TACI; eg, the antigen is a ligand is cognate to a receptor (or another, second ligand); or the antigen is a receptor and is cognate to a ligand) wherein the receptor or first ligand is capable of binding to a second antigen and the antibody is capable of binding to the second antigen (eg, BCMA when the first antigen is TACI), and wherein a multimer of the invention is capable of binding to the first and second antigens. Thus, in an example, the first antigen is TACI and the second antigen is BCMA, and the multimer of the invention is capable of binding to TACI and BCMA. In another example, the first antigen is a SARS-Cov-2 antigen (eg, spike protein antigen) and the second antigen is a SARS-Cov-1 antigen (eg, spike protein antigen), and optionally the multimer of the invention is capable of binding to the first and second antigens. In another example, the first antigen is an antigen (eg, spike protein) of a first virus and the second antigen is an antigen (eg, spike protein) of a second virus, and optionally the multimer of the invention is capable of binding to the first and second antigens. The viruses are different, eg, the viruses are Coronaviruses; eg, the viruses are different strains of influenza viruses. For example, the first and second antigens are HIV antigens (eg, for the treatment or prevention of HIV infection or a symptom thereof); or P. falciparum antigens, such first and second CSP epitopes (eg, for the treatment or prevention of malaria or a symptom thereof); or Salmonella typhimurium antigens (eg, for the treatment or prevention of Salmonella infection or a symptom thereof. In an example, a multimer of the invention specifically binds to human BCMA and human TACI, and optionally the multimer comprises a plurality (eg, 4 and no more or less than 4) of a polypeptide of the invention wherein the polypeptide comprises a BCMA binding site as disclosed herein, such as in the next paragraph. Multimers that bind in these ways can be used in any method or use disclosed herein.

The invention, thus, provides:
A method of expanding the antigen binding specificity of a binding site, wherein the binding site binds a first antigen, but not a second antigen (eg, when administered to humans) when the binding site is comprised in monovalent or bivalent form by a protein that specifically binds to the first antigen, the method comprising providing a plurality of copies of a polypeptide of the invention, and multimerising at least 4 of the polypeptides to produce a multimer comprising at least 4 copies of the polypeptide, wherein the polypeptide comprises one, two or more copies of the binding site, whereby binding sites of the multimer are capable of binding the first and second antigens.
In another example, the invention provides:
Use of a polyepeptide of the invention in a method of manufacturing a multimer for expanding the antigen binding specificity of a binding site, wherein the binding site binds a first antigen, but not a second antigen (eg, when administered to humans) when the binding site is comprised in monovalent or bivalent form by a protein that specifically binds to the first antigen, wherein the method comprises providing a plurality of copies of a polypeptide of the invention, and multimerising at least 4 of the polypeptides to produce a multimer comprising at least 4 copies of the polypeptide, wherein the polypeptide comprises one, two or more copies of the binding site, whereby binding sites of the multimer are capable of binding the first and second antigens.
For this method and use: Optionally, the polypeptide comprises a SAM which is a TD, eg, a p53 TD. Optionally, the polypeptide comprises one (and no more than one) copy of the binding site.
Optionally, the polypeptide comprises two (and no more or less than two) copies of the binding site. I an example the binding site is a VH/VL binding site or a dAb. In an example, each antigen is a cell surface receptor or ligand, eg, a human cell surface receptor or ligand. In an example, each antigen is a cell surface receptor for a common ligand.

A “4-chain antibody”, as the skilled addressee will understand, is a conventional antibody format having 2 copies of a heavy chain and 2 copies of a light chain, wherein each heavy chain is paired with a respective light chain and the heavy chain Fc regions pair to form heavy chain dimers.

The invention, by providing the ability to create multimers with broadened antigen specificity, provides useful multimers, compositions, methods and uses to target viruses whose antigens evolve through mutation during the natural history of a viral infection. In this respect, the invention may provide broadly-antigen-neutralising multimers, which can be useful for treatment or prevention of HIV infections, CoV (eg Cov-1 or Cov-2) infections or malaria.

Thus, the invention may find application to shift antigen-binding specificity of a predetermined binding site against a first antigen so that the multimer additionally or alternatively binds to a second antigen. For example, this can be demonstrated where the predetermined binding site specifically binds to BCMA, wherein the multimer of the invention binds to BCMA and TACI. For example, the predetermined binding site is the BCMA binding site of JNJ64007957 (Johnson & Johnson), AMG420 (Amgen), AMG701 (Amgen), CC-93269 (Cellgene), RGN5458, (Regeneron), PF-06863135 (Pfizer), SEA-BCMA (Seattle Genetics), MEDI2228 (AstraZeneca), belantamab (GlaxoSmithKline), idecabtagene vicleucel (Celgene), JNJ-4528 (Johnson & Johnson, Nanjing Legend Biotech), P-BCMA-01 (Poseidon Therapeutics), bb21217 (Bluebird Bio), JCARH125 (Celgene, Juno) or ALLO-715 (Allogene). Preferably, the binding site is the BCMA binding site of JNJ64007957. Preferably, the binding site is the BCMA binding site of JNJ-4528. Preferably, the binding site is the BCMA binding site of RGN5458. In an example, the multimer in this paragraph is for treating a cancer, eg, multiple myeloma.

In any aspect of the invention herein, the polypeptide of the invention optionally comprises
A: one or more epitope binding sites, optionally wherein the binding site binds to (i) a SARS-Cov-2 antigen (eg, a SARS-Cov-1 antigen and a SARS-Cov-2 antigen); (ii) BCMA (B-cell maturation antigen) and TACI (transmembrane activator and calcium modulator and cyclophilin ligand interactor); (iii) first and second Coronovirus antigens; (iv) first and second HIV antigens; (v) first and second P falciparum antigens; (vi) first and second Salmonella antigens; (vii) a TMPRSS protein (eg, a TMPRSS2 antigen); or (viii) a ACE2 antigen; or
B: one, two or more copies of an ACE2 peptide (eg, an ACE2 extracellular domain) and/or a TMPRSS2 peptide (eg, a TMPRSS protein extracellular domain); or
C: a first binding site that binds to a Coronavirus antigen (eg, a SARS-Covantigen, preferably a SARS-Cov-1 or -2 antigen) and one or more copies of an ACE2 peptide (eg, an ACE2 extracellular domain) and/or a TMPRSS2 peptide (eg, a TMPRSS protein extracellular domain); or
D: a first binding site that binds to a ACE2 extracellular domain; and one or more copies of an ACE2 peptide (eg, an ACE2 extracellular domain) and/or a TMPRSS2 peptide (eg, a TMPRSS protein extracellular domain); or
E: a first binding site that binds to a TMPRSS2 peptide extracellular domain; and one or more copies of an ACE2 peptide (eg, an ACE2 extracellular domain) and/or a TMPRSS2 peptide (eg, a TMPRSS protein extracellular domain).
Such a polypeptide may be useful for producing multimers of the invention, and such multimers may be used for any method or use disclosed herein, such as for cancer (eg, multiple myeloma) treatment when the polypepide is according to option A(ii); or for treatment or prevention of a viral (eg, Covidvirus) infection when the polypepide is according to option A ((i), (iii), (vi) or (viii) or option B, C, D or E.

In one embodiment, the multimer is useful for binding to a first epitope and a second epitope which is a mutant of the first epitope, ie, wherein the second epitope differs from the first epitope by one or more amino acids or one or more sugar residues. For example, the epitopes differ by 2, 3, 4, 5, 6, 7, 8, 9, 10, 11, 12, 13, 14, 15, 16, 17, 18, 19, 20 or more amino acids or sugar residues. As shown in Example 25, multimerization of polypeptides described herein may produce multimers that can use the same binding site to bind more than one (eg, 2) different epitopes or antigens. Thus, in an example, the first epitope is comprised by a first antigen and the second epitope is comprised by a second antigen, eg, the antigens are different receptors (such as cell-surface receptors), or different ligands (such as different forms of spike protein of a virus). Optionally, the virus is a SARS virus (eg, SARS-Cov, SARS-Cov-2 or MERS-Cov), HIV or influenza virus. Optionally, the virus is HIV and the epitope is an Env epitope, gp41 epitope or gp120 epitope. Optionally, the virus is influenza virus and the epitope is an epitope of haemagglutinin or matrix protein 2. By providing a multimer that is capable of binding to different forms of a spike protein of such a virus, the multimer is useful for treating or preventing or reducing a seasonal viral infection in humans or animals. For example, the multimer is useful for treating, preventing or reducing infection by a virus comprising a first form of spike protein, and the multimer is useful for treating, preventing or reducing infection by a virus comprising a second form of the spike protein. Thus, in this way the multimer is useful for treating or preventing viral infection in a first and second season wherein humans are infected in the first season by the virus comprising the first spike form and humans are infected in the second season by the virus comprising the second spike form. Instead of a spike protein, the epitope may be a different virus antigen, such as a capsid or tail protein. The multimer, therefore, is capable of binding to different strains of a virus and preferably neutralises the virus (eg, renders it non-infective and/or reduces proliferation of the virus). Thus, in an embodiment the invention provides a seasonal virus treatment or prophylaxis medicament for administration to a human or animal subject, wherein the medicament comprises a plurality of multimers of the invention, such as multimers according to this paragraph, wherein the medicament comprises a pharmaceutically acceptable diluent, carrier or excipient. Such diluents, carriers and excipients are well known to the skilled person. Administration is, eg, intravenous, inhaled, oral or intranasal administration. The invention therefore provides in an embodiment: a multi-seasonal (eg, 2-seasonal or 3-seasonal) anti-viral medicament comprising a plurality of multimers of the invention which are capable of binding to first and second strains of the virus, wherein the strains differ in a surface-exposed antigen to which the multimers can bind. In an example, the seasons are a first year and a second year (eg, two consecutive years or two consecutive winters thereof, or two consecutive summers thereof, or two consecutive springs thereof, or two consecutive falls/autumns thereof). Optionally in any embodiment herein, the virus is a SARS virus (eg, SARS-Cov, SARS-Cov-2, MERS-Cov), HIV, ebola virus, zika virus, norovirus, rotovirus, respiratory synctial virus (RSV), an exanthematous virus, papilloma virus, hepatitis (eg, A, B, C, D or E) virus, Lassa fever virus, dengue fever virus, yellow fever virus, Marburg fever virus, Crimean-Congo fever virus, polio virus, viral meningitis virus, viral encephalitis virus, rabies virus, smallpox virus, hantavirus or influenza virus. When the multimer is useful or used in a method for reducing the virus in an animal, this is advantageous for reducing a zoonotic population of viruses that are transmissible to humans, wherein the viruses are capable of causing a disease or condition (or death) in humans. In this respect, the animal may be a livestock animal, such as a pig, poultry (eg, chicken, duck or turkey), sheep, cow, goat, fish or shellfish. In an example, the animal is a bat, racoon dog, dog, cat, palm civet or camelid (eg, a camel or dromedary). In an example, the animal is a bird.

By presenting multiple copies of the epitope(s) multimers of the invention are believed to provide useful means for vaccination and for stimulating strong immune responses in a human or animal. Thus, in an alternative, the multimers comprise a plurality (eg, 4, 8, 12, 16 or 20) of copies of a peptide, wherein the peptide comprises an epitope of a pathogen, such as a surface-exposed epitope of a virus or bacterium. For example, the peptide comprises a first and/or second epitope as described in the immediately preceding paragraph. In this way, there is provided a vaccine composition for administration to a human or animal for preventing or reducing an infection by the virus or bacterium. In an example, each polypeptide of the multimer comprises a first peptide comprising a first said epitope of the pathogen and a second peptide comprising a second said epitope of the pathogen. Optionally, the polypeptide comprises 2, 3, 4, 5, 6, 7, 8, 9 or 10 (eg, 2 or 3) said epitopes of the pathogen. Optionally, the polypeptide comprises 2, 3, 4, 5, 6, 7, 8, 9 or 10 (eg, 2 or 3) different epitopes of the pathogen.

For example, the virus is Coronavirus, eg, SARS-Cov, SARS-Cov-2, a SARS-related coronavirus (a SARSr-Cov), HCoV-OC43, HCoV-HKU1, HCoV-NL63, HCoV-229E. In an example, the virus is SARS-CoV ZXC21, ZC45, RaTG13, CUHK-W1, Urbani, GZ02, A031, A022, WIV16, WIV1, Rp3, Rs672 or HKU4. For example, the virus is Coronavirus is a group 1, group 2 or group 3 Coronavirus. For example, the multimer is a vaccine antigen composition comprising copies of a polypeptide of the invention. In an aspect, the polypeptide comprises one or more S epitopes of said virus. In an aspect, the polypeptide of the invention comprises a S1 and/or S2 epitope of said virus; or a S^{A} and/or S^{B} epitope of said virus (eg, a SARS-Cov-2 S^{A} and/or S^{B} epitope, preferably SARS-Cov-2 S^{B} epitope). In an aspect, the polypeptide comprises a peptide which comprises all or part of the S^{A} domain and/or all or part of the S^{B} domain. In an aspect, the polypeptide comprises a peptide which comprises all or part of the S^{B} domain and all or part of the S2 subunit, and optionally also the S 1/S2 boundary. Additionally or alternatively, the polypeptide comprises a peptide which comprises the virus spike protein S1 subunit/ S2 subunit boundary. Additionally or alternatively, the polypeptide comprises a peptide which comprises the virus spike protein furin cleavage site. In an alternative, the multimer comprises a plurality of binding sites for one or more of the epitopes, wherein the multimer comprises copies of a polypeptide of the invention wherein the polypeptide comprises one or more epitope binding sites, each epitope being an epitope as described in this paragraph.

In an example, the SARS-Cov epitope comprises one or more N-linked glycans, eg, where each N is an N selected from the following table or is a corresponding N in the virus.

Conservation of N-Linked Glycosylation Sequons in SARS-CoV-2 S and SARS-CoV S

| | |
|---|---|
| SARS-CoV-2 S | SARS-CoV S |
| N₁₇LT | T₂₁FD |
| P₂₅PA | N₂₉YT |
| N₆₁VT | N₆₅VT |
| H₆₉VS | N₇₃HT |
| N₇₄GT | ... |
| S₁₁₂KT | N₁₀₉KS |
| N₁₂₁NA | N₁₁₈NS |
| N₁₂₂AT | N₁₁₉ST |
| N₁₄₉KS | T₁₄₆QT |
| N₁₆₅CT | N₁₅₈CT |
| N₂₃₄IT | N₂₂₇IT |
| N₂₈₂GT | N₂₆₉GT |
| N₃₃₁IT | N₃₁₈IT |
| N₃₄₃AT | N₃₃₀AT |
| N₃₇₀SA | N₃₅₇ST |
| N₆₀₃TS | N₅₈₉AS |
| N₆₁₆CT | N₆₀₂CT |
| N₆₅₇NS | D₆₄₃TS |
| N₇₀₉NS | N₆₉₁NT |
| N₇₁₇FT | N₆₉₉FS |
| N₈₀₁FS | N₇₈₃FS |
| N₁₀₇₄FT | N₁₀₅₆FT |
| N₁₀₉₈GT | N₁₀₈₀GT |
| N₁₁₃₄NT | N₁₁₁₆NT |
| N₁₁₅₈HT | N₁₁₄₀HT |
| N₁₁₇₃AS | N₁₁₅₅AS |
| N₁₁₉₄ES | N₁₁₇₆ES |

Italic font indicates the absence of a glycosylation sequon and deletions are indicated with periods. Glycans observed in the SARS-CoV-2 S cryo-EM map are underlined. (see Walls AC, Park YJ, Tortorici MA, Wall A, McGuire AT, Veesler D. Structure, Function, and Antigenicity of the SARS-CoV-2 Spike Glycoprotein [published online ahead of print, 2020 Mar 6]. Cell. 2020;S0092-8674(20)30262-2. doi: 10.1016/j.cell.2020.02.058, the disclosure of which is incorporated herein by reference.

Position numbers are with reference to SARS-CoV-2 sequence with Accession Number: YP_009724390.1; and SARC-CoV Urbani sequence with Accession Number: AAP13441.1.

In an example, the virus is selected from SARS-CoV-2 (YP_009724390.1), SARSr-CoV RaTG13 (QHR63300.2), SARS-CoV Urbani (AAP13441.1), SARS-CoV CUHK-W1 (AAP13567.1), SARS-CoV GZ02 (AAS00003.1), SARS-CoV A031 (AAV97988.1), SARS-CoV A022 (AAV91631.1), WIV-16 (ALK02457.1), WIV-1 (AGZ48828.1), SARSr-CoV ZXC21 (AVP78042.1), SARSr-CoV ZC45 (AVP78031.1), SARSr-CoV Rp3 (Q3I5J5.1), SARSr-CoV Rs672 (ACU31032.1). Accession numbers are shown in brackets; the sequences thereof are explicitly incorporated herein by reference for use in the present invention, eg, for providing epitope or antigen sequence.

For example, the pathogen is HIV and the polypeptide comprises a gp120 epitope and a gp41 epitope, eg, the polypeptide comprises a SOSIP peptide.

For example, the pathogen is a Coronavirus (eg, SARS-Cov, SARS-Cov-2 or MERS-Cov) and the polypeptide comprises a first spike epitope and a second spike epitope, wherein the epitopes are different from each other. For example, the first epitope and/or second epitope comprises a sugar residue. For example, the first epitope comprises a viral contact residue for ACE2 and/or the second epitope comprises a viral contact reside for TMPRSS2 and optionally the virus is SARS-Cov or SARS-Cov-2. For example, the first epitope comprises a viral contact residue for DPP4 and/or the second epitope comprises a viral contact reside for TMPRSS2 and optionally the virus is MERS-Cov.

For example, the pathogen is influenza and the polypeptide comprises one or more haemagglutinin epitopes. Optionally, any influenza herein is influenza A, B or H1N1.

For example, the pathogen is HIV and the polypeptide comprises a plurality of Env epitopes.

For example, the pathogen is a virus (eg, a Coronavirus) and the polypeptide comprises a plurality of spike epitopes.

For example, the pathogen is a virus and the polypeptide comprises a plurality of capsid or tail epitopes.

In an example, the virus is a bacteriophage that is capable of infecting a host bacterial cell; or the virus is a virus that is capable of infecting an archaeal cell.

In an example, the pathogen is a bacterium selected from
Bacillus anthracis
Bacillus cereus
Bartonella henselae
Bartonella quintana
Bordetella pertussis
Borrelia burgdorferi
Borrelia garinii
Borrelia afzelii
Borrelia recurrentis
Brucella abortus
Brucella canis
Brucella melitensis
Brucella suis
Campylobacter jejuni
Chlamydia pneumoniae
Chlamydia trachomatis
Chlamydophila psittaci
Clostridium botulinum
Clostridium difficile
Clostridium perfringens
Clostridium tetani
Corynebacterium diphtheriae
Enterococcus faecalis
Enterococcus faecium
Escherichia coli
Francisella tularensis
Haemophilus influenzae
Helicobacter pylori
Legionella pneumophila
Leptospira interrogans
Leptospira santarosai
Leptospira weilii
Leptospira noguchii
Listeria monocytogenes
Mycobacterium leprae
Mycobacterium tuberculosis
Mycobacterium ulcerans
Mycoplasma pneumoniae
Neisseria gonorrhoeae
Neisseria meningitidis
Pseudomonas aeruginosa
Rickettsia rickettsii
Salmonella typhi
Salmonella typhimurium
Shigella sonnei
Staphylococcus aureus
Staphylococcus epidermidis
Staphylococcus saprophyticus
Streptococcus agalactiae
Streptococcus pneumoniae
Streptococcus pyogenes
Treponema pallidum
Ureaplasma urealyticum
Vibrio cholerae
Yersinia pestis
Yersinia enterocolitica; and
Yersinia pseudotuberculosis

The invention provides a protein comprising 4, 12, 16, 20, 24, 28 or 32 copies of an epitope disclosed herein, optionally also comprising 4, 12, 16, 20, 24, 28 or 32 copies of a second second epitope disclosed herein. Optionally, the protein is useful as a vaccine for treating or preventing an infection of a virus or bacterium in a human or animal subject, wherein the virus or bacterium comprises the epitope(s). Optionally, the protein is a multimer as disclosed herein, the multimer comprising four copies of a polypeptide, wherein the polypeptide comprises 1, 2, 3, 4, 5, 6, 7, or 8 copies of the first epitope (and optionally comprises 1, 2, 3, 4, 5, 6, 7, or 8 copies of the second epitope). In an alternative, the invention provides a protein comprising 4, 12, 16, 20, 24, 28 or 32 copies of a binding site that is capable of binding to an epitope disclosed herein, optionally also comprising 4, 12, 16, 20, 24, 28 or 32 copies of a second binding site that is capable of binding to a second epitope disclosed herein. Optionally, the protein is useful as a therapy for treating or preventing an infection of a virus or bacterium in a human or animal subject, wherein the virus or bacterium comprises the epitope(s). Optionally, the protein is a multimer as disclosed herein, the multimer comprising four copies of a polypeptide, wherein the polypeptide comprises 1, 2, 3, 4, 5, 6, 7, or 8 copies of the first epitope (and optionally comprises 1, 2, 3, 4, 5, 6, 7, or 8 copies of the second epitope). In an example, the protein is useful as an assay reagent for detecting a virus of bacterium comprising the epitope(s). To this end there is provided a first method of detecting the virus or bacterium in a sample (eg, in vitro), the method comprising contacting the sample with the protein to allow the protein to bind to one or more copies of the virus or bacterium in the sample, and detecting the binding, eg, using a detection reagent that binds to virus or bacteria that have bound to the protein. There is also provided a method of detecting antibodies that are capable of binding (and optionally neutralising) the virus or bacterium in a sample (eg, in vitro), the method comprising contacting the sample with the protein to allow the protein to bind to such antibodies in the sample, and detecting the binding, eg, using a detection reagent that binds to the antibodies that have bound to the protein. The detection reagent may be an anti-virus or bacterium agent (such as a labelled antibody) in the first method; or an anti-antibody (eg, anti-IgG or anti-IgM) agent (such as a labelled antibody) in the second method. The label may, for example, be a fluorescence label, eg, GFP. The sample may be a blood, spit, sputum or cell sample, eg, a patient sample, such as a patient that is suffering from, is suspected of suffering from or has suffered from an infection by the virus or bacterium. In an example, the protein or multimer of the invention is immobilised on a solid surface, eg, a petri dish or test tube surface, or a flow chamber surface. For example, the surface is a particle surface, eg, a bead surface, such a magenetic bead, magnetisable bead, metal or ferrous bead. In an example, the protein or multimer of the invention is comprised by a fluid, eg, a liquid, eg, a liquid in a droplet, such as an emulsion droplet. Thus, the protein or multimer is useful in a microfluidics method of detecting the virus, bacterium or antibody (eg, IgG or IgM that binds the virus or bacterium).

In an example, the polypeptide comprises one or more (eg, 1, 2, 3 or 4) protein G peptides each of which is capable of binding to IgG, or the protein or multimer comprises a plurality of such polypeptides. Such a multimer or protein is useful to capture IgG when the protein or sample is contacted with a sample (eg, blood, sputum, saliva, semen or cell sample), such as wherein the contacting is carried out in vitro, such as in an in vitro assay. The avidity effect of the multimer’s plurality of protein G peptides is useful to enhance IgG detection sensitivity . The invention, therefore, provides such an assay method and a kit comprising the protein or multimer (optionally immobilised on a solid surface, such as on the surface of a container) and a detection reagent. In an example, the reagent comprises an antigen or epitope that is bound (eg, specifically bound) by the captured IgG. Optionally, the epitope is a viral or bacterial epitope, eg, a viral spike, capsid or tail fibre epitope; or eg, a bacterial cell surface epitope. Optionally, the epitope is a virus spike epitope, eg, a Coronavirus spike epitope, such as a SARS-CoV or SARS-Cov-2 or MERS-CoV spike epitope. Optionally, the reagent comprises a label that is detectable, such as a fluorescence marker, eg, GFP or an Alexa fluor marker. Instead of a protein G peptide, additionally or alternatively the polypeptide, multimer or protein comprises one or more protein A peptides that are each capable of binding to an antibody Fc, such as a Fc of an anti-viral or anti-bacterial antibody from a patient sample. Instead of a protein G peptide, additionally or alternatively the polypeptide, multimer or protein comprises one or more protein L peptides that are each capable of binding to an antibody light chain, such as a light chain of an anti-viral or anti-bacterial antibody from a patient sample (eg, an IgG, IgM, IgA, IgE or IgD antibody).

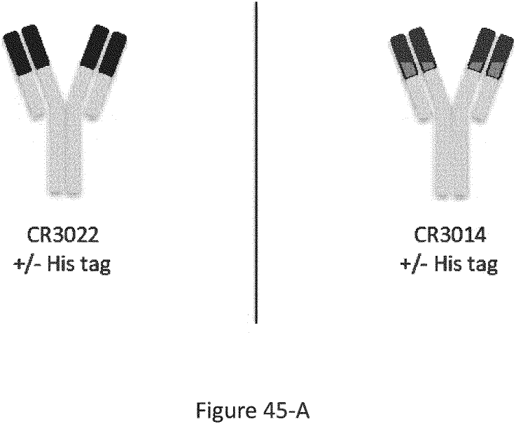

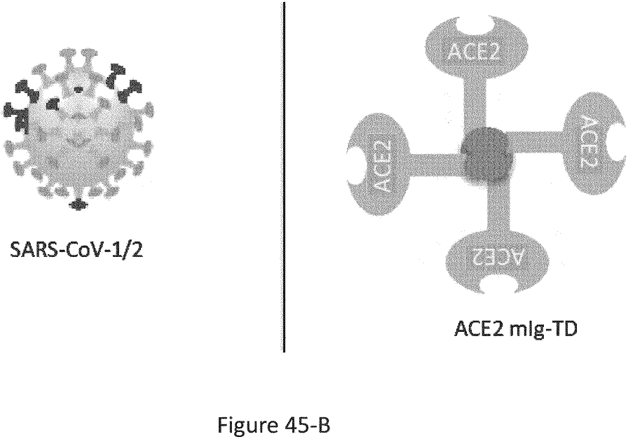

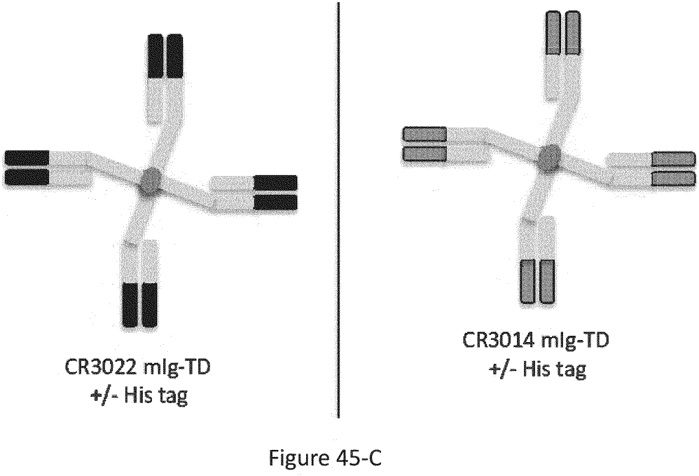

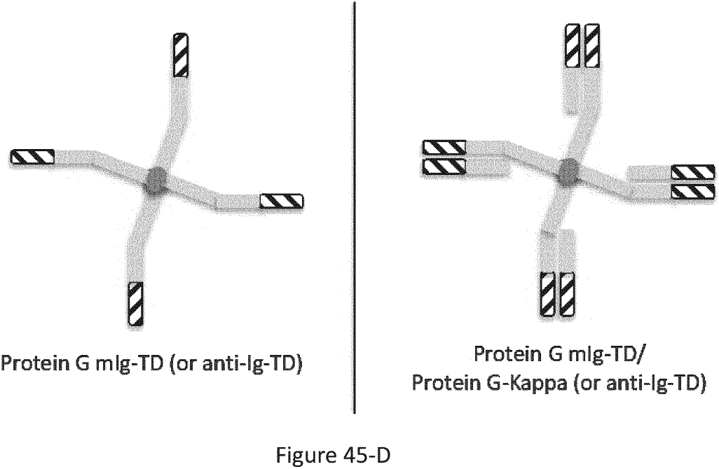

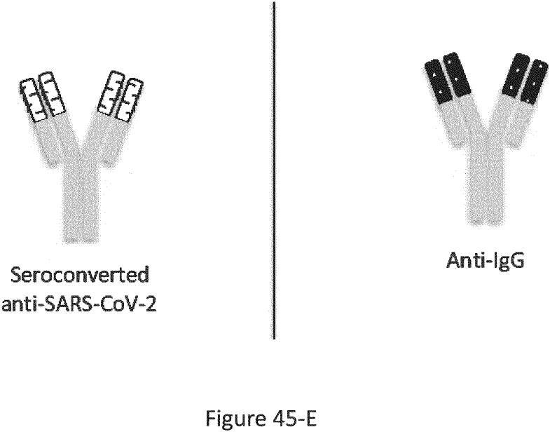

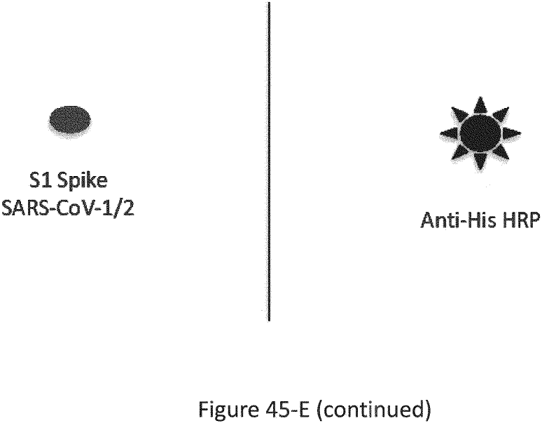

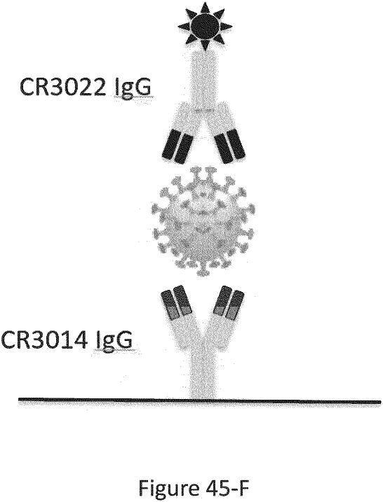

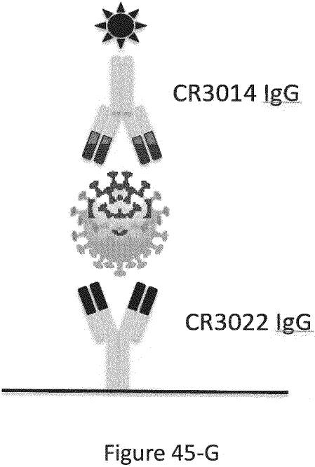

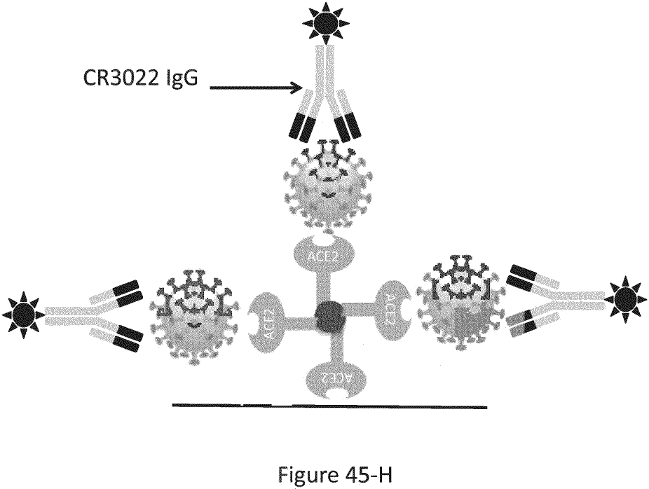

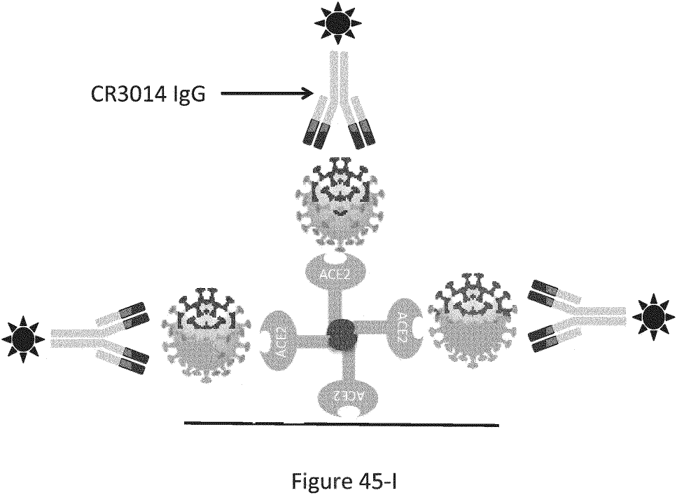

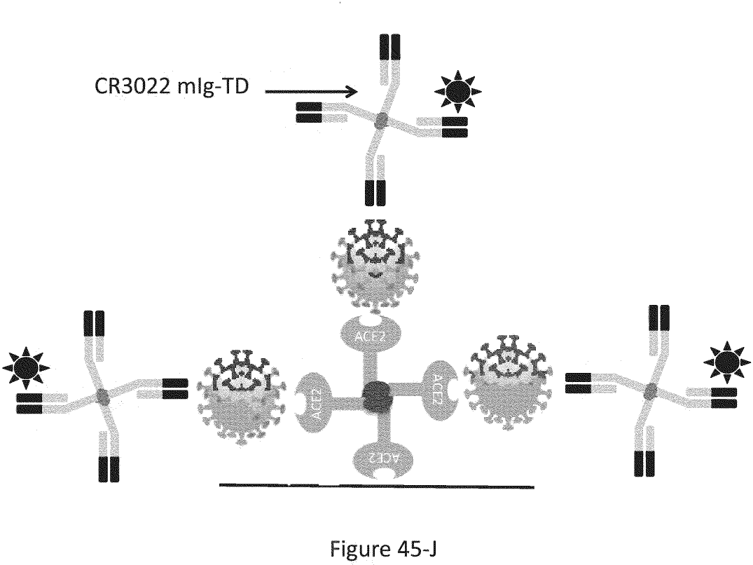

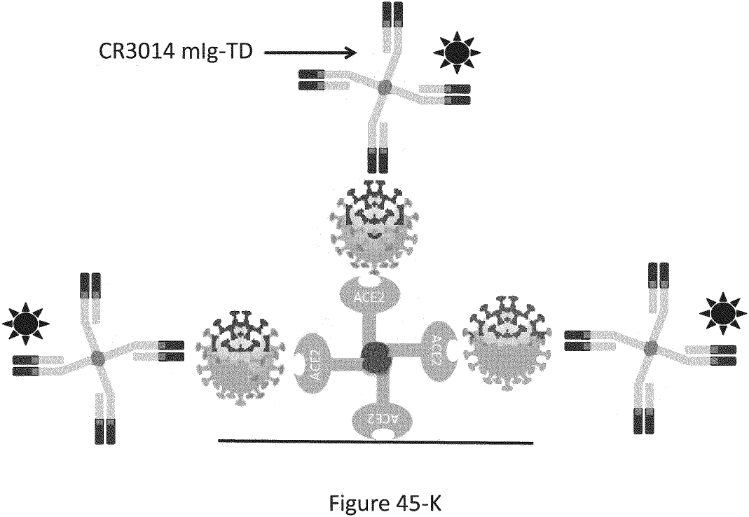

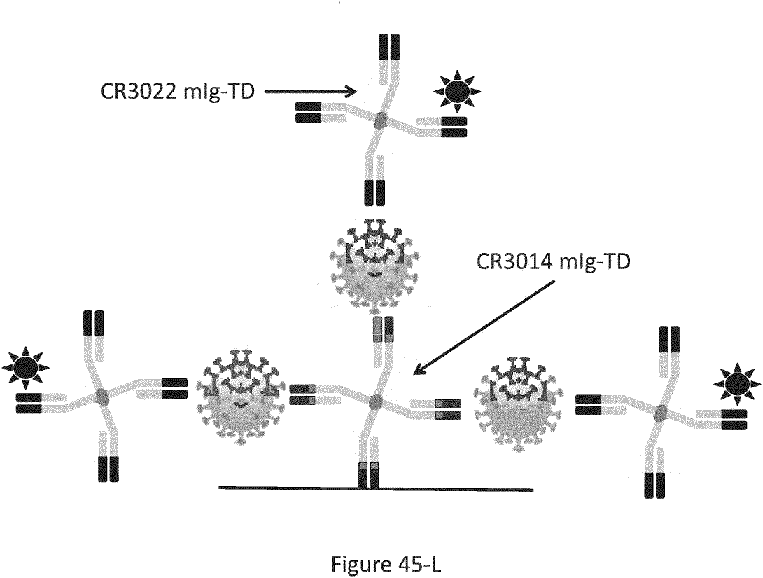

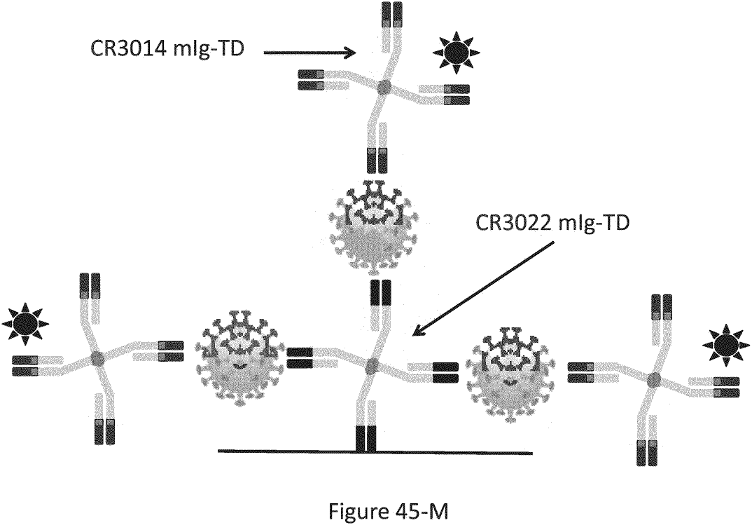

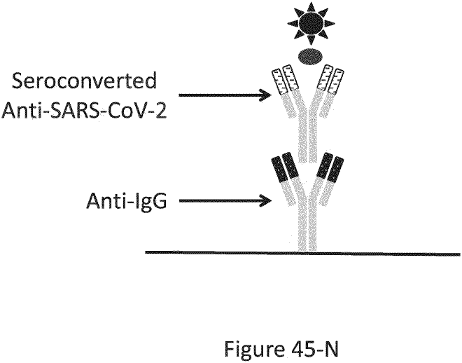

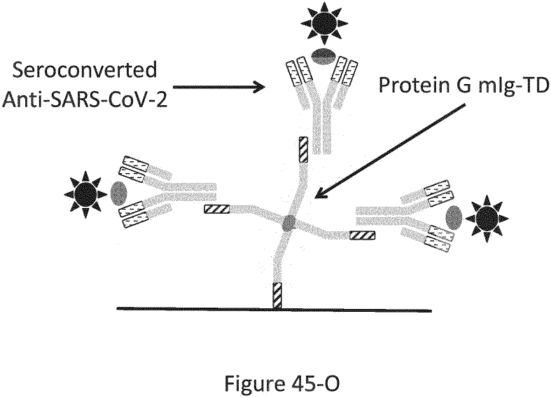

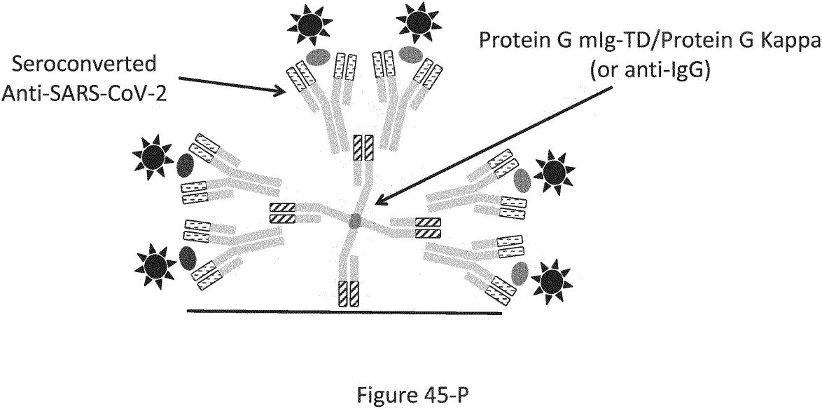

See FIG. 45. The invention provides any reagent or combination of reagents disclosed in that figure, such as the reagent of any one of A-E or the configuration of reagents disclosed in any one of F to P.

Optionally, where the protein or multimer comprises binding sites for ACE2, the protein or multimer may be used for treating or preventing hypertension in a human or animal subject. Optionally, the protein or multimer comprises one or more ACE2 epitopes, wherein the protein or multimer may be used for treating or preventing hypertension in a human or animal subject, such as by administration of the protein or multimer to the subject to raise antibodies against ACE2 in the subject. Alternatively, the treatment or prevention is an inflammatory condition (eg, lung inflammation), pneumonia, COPD, asthma or any treatment or prevention of a condition disclosed in US20110020315A1, the disclosure of which is incorporated herein by reference.

Optionally, where the protein or multimer comprises binding sites for TMPRSS2, the protein or multimer may be used for treating or preventing a cancer (eg, prostate cancer) or viral infection (eg, influenza infection) in a human or animal subject. Optionally, the protein or multimer comprises one or more TMPRSS2 epitopes, wherein the protein or multimer may be used for treating or preventing a cancer (eg, prostate cancer) or viral infection (eg, influenza infection) in a human or animal subject, such as by administration of the protein or multimer to the subject to raise antibodies against TMPRSS2in the subject. Alternatively, the treatment or prevention is any treatment or prevention of a condition disclosed in US 9,498,529, the disclosure of which is incorporated herein by reference. For example, the inflammation is local inflammation of a tissue or an organ and/or a systemic inflammation. For example, the inflammation comprises sepsis. For example, the inflammation comprises an autoimmune disease.

Optionally, where the protein or multimer comprises copies of a binding site for a virus spike, the binding site may be the binding site of antibody 80R, CR3014, CR3006, CR3013 or CR3022. The VH and VL domain sequences of these antibodies are incorporated herein by reference for possible inclusion in a protein or multimer of the invention. Optionally, where the protein or multimer comprises copies of a binding site for a virus spike, the binding site is capable of binding to amino acid residues 426-492, 318-510, or 318-510 of S1 subunit of SARS-CoV, and wherein optionally the protein or multimer binds SARS-CoV and SARS-CoV-2.

Optionally, where the protein or multimer herein is for treating or preventing viral pneumonia in a human or animal subject, eg, wherein the subject is suffering from or is at risk of suffering from a Coronavirus infection. Optionally, where the protein or multimer herein is for treating or preventing Coronavirus viral pneumonia in a human or animal subject. Optionally, where the protein or multimer herein is for treating or preventing Coronavirus viral pneumonia in a human or animal subject, wherein the binding sites are capable of binding to a Pseudomonoas aeruginosa epitope, or wherein the protein or multimer comprises Pseudomonoas aeruginosa epitopes.

Optionally, where the protein or multimer herein is for treating or preventing a viral infection or symptom thereof in a human or animal subject, wherein the binding sites are capable of binding to a Cathepsin L epitope, or wherein the protein or multimer comprises Cathepsin L epitopes. In an example, the virus is Ebola virus or a SARS virus or a Coronavirus (eg, SARS-CoV or SARS-CoV-2).

Optionally, where the protein or multimer herein is for treating or preventing a Coronavirus infection or symptom thereof in a human or animal subject, wherein the binding sites are capable of binding SARS-CoV S1 RBD or RBDR, or wherein the protein or multimer comprises SARS-CoV S1 RBD or RBDR. The receptor-binding determining region (RBDR) that recognizes ACE2. For example, the binding sites are capable of binding the peptide S₄₇₁₋₅₀₃ of the RBD; or the protein or multimer comprises copies of S₄₇₁₋₅₀₃ of the RBD.

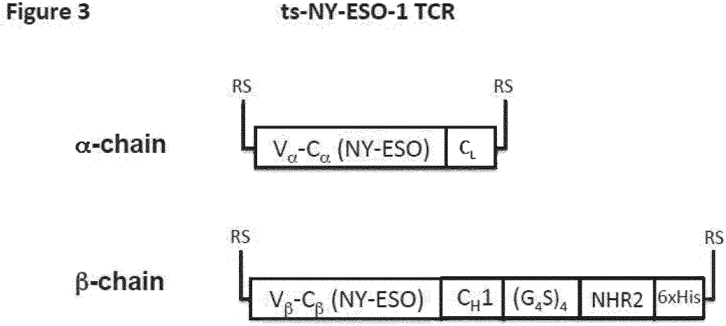

Optionally, where the protein or multimer herein is for treating or preventing a Coronavirus infection or symptom thereof in a human or animal subject, wherein the binding sites are capable of binding SARS-CoV-2 S1 RBD or RBDR, or wherein the protein or multimer comprises SARS-CoV-2 S1 RBD or RBDR. The receptor-binding determining region (RBDR) that recognizes ACE2. For example, the binding sites are capable of binding the peptide S₄₇₁₋₅₀₃ (ALNCYWPLNDYGFYTTTGIGYQPYRVVVLSFEL) (SEQ ID NO: 500) of the RBD; or the protein or multimer comprises copies of S₄₇₁₋₅₀₃ of the RBD. In an example, the protein or multimer comprises copies of a peptide comprises by the amino acid sequence from position 318 to 536 of SARS-CoV or the equivalent amino acid sequence of SARS-CoV-2, wherein the peptide comprises the amino acid sequence from position 424 to position 494. Optionally, the peptide is RBD219-N1 (see For example, Chen, W.; Hotez, P.J.; Bottazzi, M.E. Potential for Developing a SARS-CoV Receptor Binding Domain (RBD) Recombinant Protein as a Heterologous Human Vaccine against Coronavirus Infectious Disease (COVID)-19. Preprints 2020, 2020020449, the sequences of (i) RBD219-N1, (ii) the sequence in the blue box (the epitope consisting S343-367, 373-390 and 411-428 (reported by Bian et al)) and (iii) the sequences in the green box from the first N to QPY for each of RBD219-N1 and SARS-CoV-2 spike shown in FIG. 1 each is incorporated herein by reference for possible use in the invention (eg, the protein or multimer of the invention comprises one or more binding sites that binds to a said peptide of (i), (ii) or (iii); or the protein or multimer comprises one or more of peptides (i), (ii) and (iii)). Optionally, the protein or multimer of the invention comprises one or more copies of the antigen binding site of an antibody shown in Table 1 or 2 of Chen, W.; Hotez, P.J.; Bottazzi, M.E., “Potential for Developing a SARS-CoV Receptor Binding Domain (RBD) Recombinant Protein as a Heterologous Human Vaccine against Coronavirus Infectious Disease (COVID)-19”, Preprints 2020, 2020020449; or shown in Table 1 or 2 of Asian Pac J Allergy Immunol. 2020 Mar;38(1): 10-18. doi: 10.12932/AP-200220-0773, “Perspectives on monoclonal antibody therapy as potential therapeutic intervention for Coronavirus disease-19 (COVID-19)”, Shanmugaraj B et al; or shown in Table 1 of Zhou G, Zhao Q. Perspectives on therapeutic neutralizing antibodies against the Novel Coronavirus SARS-CoV-2. Int J Biol Sci. 2020;16(10):1718-1723, Published 2020 Mar 15, doi:10.7150/ijbs.45123; or shown in Table 1 of Trends in Immunology 2020, “Neutralizing Antibodies against SARS-CoV-2 and Other Human Coronaviruses”, Shibo Jiang, Christopher Hillyer, and Lanying Du (https://www.cell.com/trends/immunology/fulltext/S1471-4906(20)30057-0?rss=yes), the disclosures of all of which (including the VH and VL sequences of said antibodies) are incorporated herein by reference for use in the invention. Optionally, the protein or multimer comprises copies of a peptide, wherein the peptide comprises (i) 1, 2, 3, 4, 5, 6, 7, 8, 9, 10, 11, 12, 13 or 14 amino acids corresponding to amino acids selected from 415T, 439N, 449Y, 453Y, 455L, 486F, 487N, 489Y, 493Q, 498Q, 500T, 501N, 502G and 505Y of SARS-CoV-2 (eg, SARS-CoV-2 (YP_009724390.1)), (ii) amino acids corresponding to amino acids 486F, 487N, 489Y, 493Q, 498Q, 500T, 501N, 502G and 505Y of SARS-CoV-2 (eg, SARS-CoV-2 (YP_009724390.1)), (iii) an amino acid corresponding to amino acid 415T of SARS-CoV-2 (eg, SARS-CoV-2 (YP_009724390.1)), or (iv) amino acids corresponding to amino acids 439N, 449Y, 453Y, 455L of SARS-CoV-2 (eg, SARS-CoV-2 (YP_009724390.1)). Optionally, the protein or multimer comprises copies of a peptide, wherein the peptide comprises (i) 1, 2, 3, 4, 5, 6, 7, 8, 9, 10, 11, 12, 13 or 14 amino acids corresponding to amino acids selected from 415T, 439R, 449Y, 453Y, 455Y, 486L, 487N, 489Y, 493N, 498Y, 500T, 501T, 502G and 505Y of SARS-CoV (eg, SARS-CoV Urbani), (ii) amino acids corresponding to amino acids 486L, 487N, 489Y, 493N, 498Y, 500T, 501T, 502G and 505Y of SARS-CoV (eg, SARS-CoV Urbani), (iii) an amino acid corresponding to amino acid 415T of SARS-CoV (eg, SARS-CoV Urbani), or (iv) amino acids corresponding to amino acids 439R, 449Y, 453Y, 455Y of SARS-CoV (eg, SARS-CoV Urbani). Optionally, the protein or multimer comprises copies of binding site that binds to a peptide, wherein the peptide comprises (i) 1, 2, 3, 4, 5, 6, 7, 8, 9, 10, 11, 12, 13 or 14 amino acids selected from 415T, 439N, 449Y, 453Y, 455L, 486F, 487N, 489Y, 493Q, 498Q, 500T, 501N, 502G and 505Y of SARS-CoV-2 (eg, SARS-CoV-2 (YP_009724390.1)), (ii) amino acids c 486F, 487N, 489Y, 493Q, 498Q, 500T, 501N, 502G and 505Y of SARS-CoV-2 (eg, SARS-CoV-2 (YP_009724390.1)), (iii) amino acid 415T of SARS-CoV-2 (eg, SARS-CoV-2 (YP_009724390.1)), or (iv) amino acids 439N, 449Y, 453Y, 455L of SARS-CoV-2 (eg, SARS-CoV-2 (YP_009724390.1)). Optionally, the protein or multimer comprises copies of a comprises copies of binding site that binds to a peptide, wherein the peptide comprises (i) 1, 2, 3, 4, 5, 6, 7, 8, 9, 10, 11, 12, 13 or 14 amino acids selected from 415T, 439R, 449Y, 453Y, 455Y, 486L, 487N, 489Y, 493N, 498Y, 500T, 501T, 502G and 505Y of SARS-CoV (eg, SARS-CoV Urbani), (ii) amino acids 486L, 487N, 489Y, 493N, 498Y, 500T, 501T, 502G and 505Y of SARS-CoV (eg, SARS-CoV Urbani), (iii) amino acid 415T of SARS-CoV (eg, SARS-CoV Urbani), or (iv) amino acids 439R, 449Y, 453Y, 455Y of SARS-CoV (eg, SARS-CoV Urbani). Optionally, the binding sites are capable of binding the P6 peptide (EEQAKTFLDKFNHEAEDLFYQSSGLGKGDFR) (SEQ ID NO: 501) of the RBD; or the protein or multimer comprises copies of the P6 peptide (EEQAKTFLDKFNHEAEDLFYQSSGLGKGDFR) of the RBD. In an example, the binding site is the antigen binding site of an antibody selected from 80R, m396, F26G19, s230, CR3014, and CR3022. See, eg, Shah et al, doi: 10.21203/rs.3.rs-16932/vl, “Sequence variation of SARS-CoV-2 spike protein may facilitate stronger interaction with ACE2 promoting high infectivity” and supplementary materials, which are incorporated herein by reference. The binding site of the invention may, for example, comprise the VH/VL or scFv of antibody A, B, C, D or E in FIG. 3 of this reference, the sequence of which is incorporated herein by reference for use in the invention; and optionally the virus is a Coronavirus, such as SARS-CoV or SARS-CoV-2.

Optionally, the protein or multimer of the invention is for treating or preventing HIV and comprises one or more copies of the antigen binding site of an antibody shown in Table 1 or FIG. 4 of Annu. Rev. Immunol. 2016. 34:635-59, doi: 10.1146/annurev-immunol-041015-055515, “Broadly Neutralizing Antibodies to HIV and Their Role in Vaccine Design”, Dennis R. Burton and Lars Hangartner; the disclosures of all of which (including the VH and VL sequences of said antibodies) are incorporated herein by reference for use in the invention.

In an example, the protein or multimer comprises a plurality of (eg, 4, 8, 12, 12, 16, 20, 24, 28 or 32) copies of a peptide disclosed herein, eg, a peptide disclosed in the immediately preceding paragraph.

Optionally, where the protein or multimer herein is for treating or preventing a RSV infection or symptom thereof in a human or animal subject, wherein the binding sites are palivizumab binding sites.

Optionally, the binding site of the protein, multimer or polypeptide of the invention disclosed herein comprises a binding site for a peptide or epitope disclosed herein, or for a peptide or epitope whose amino acid sequence is at least 70, 75, 80, 85, 90, 95, 96, 97, 98 or 99% identical to the amino acid sequence of a peptide or epitope disclosed herein. Alternatively, the protein, multimer or polypeptide binding site competes (eg, in SPR) with a binding site disclosed in the first sentence of this paragraph.

In one configuration, the invention provides a RNA (eg, mRNA or self-amplifying mRNA, or saRNA) that encodes a polypeptide or protein of the invention. In an example, there is provided a medicament (eg, a vaccine) comprising the RNA, wherein the RNA is for administration to a human or animal subject for treating or preventing a disease or condition in the subject, wherein the RNA is expressed in the subject to produce polypeptides, proteins or multimers of the invention. Optionally, the medicament is a vaccine and the condition is a virial or bacterial infection, such as when the encoded polypeptide comprises an epitope of the virus or bacterium or a binding site that is capable of binding to such an epitope.

The polypeptide, protein or multimer can comprise multiple (i.e. 2, 3 or 4) different peptides of the target virus or bacterium (eg, peptides of cell-surface proteins) for use as vaccine. In another embodiment, there is provided a composition comprising first and a second multimer or protein of the invention wherein the multimers/proteins comprise peptides of the target virus or bacterium (eg, peptides of cell-surface proteins) for use as vaccine, wherein the peptides of the first multimer or protein differ from the peptides of the second multimer or protein. For example, the proteins or multimers do not comprise a common such peptide. For example, the second multimer or protein comprises a such peptide that is not comprised by the first protein or multimer. Optionally, the composition comprises a third protein or multimer which is different from the first and second proteins/multimers, wherein the third protein or multimer comprises a said peptide that is not comprised by the first and second proteins/multimers.

For a vaccine herein that targets a virus, the peptide or epitope is not limited to a spike epitope (eg, S1 or S2 subunit epitope); the virus epitope could be from any region of the virus, preferably a region that is exposed on the cell surface of the viral host.

Multimerizing virus or bacterial peptides or epitopes according to the invention may advantageously enhance immunogenicity in the subject and thus promote generation of anti-viral/bacterial antibodies that are desirably affinity matured and may give rise to antibodies with a broad epitope coverage (ie, more recognising more than one epitope) of the virus/bacterium.

### Providing New Specifity by Multimerisation & Cross-Reactive Multimers

Through multimerization made possible by the invention, certain aspects advantageously enable provision of new antigen specificities for binding sites; or new cross-reactivity of binding sites (eg, wherein a binding site in a control binds, such as an IgG, a first but not second antigen, but when present as a multimer of the invention binds both antigens - as exemplified herein). The multimerization also or alternatively can greatly enhance binding strength for an antigen, such as a viral antigen, thereby providing multimer format that are useful for human or animal therapy and for highly sensitive assays, eg, to detect antigen or virus in a sample, such as a serum sample of a subject. Again, such highly sensitive assaying is exemplified herein. Thus, the invention may render therapeutically- or prophylactically-useful a binding site that has hitherto been useless for therapy of prophylaxis of a disease or condition (eg, infection by a certain virus) in humans or animals. As exemplified herein, the multimerization of the invention converts binding based on anti-SARS-CoV-2 binding sites from therapeutically- or prophylactically-useless to therapeutically- or prophylactically-useful for administration of the multimer of the invention to a human or animal subject for treating (eg, reducing) or preventing a SARS-CoV-2 infection. Thus, the invention enables re-purposing of pre-existing antigen binding sites to provide for possible new applications for treatement, prevention or detection of a disease, condition or infection.

In a first aspect, the invention provides:

A protein multimer (first multimer) comprising more than 2 copies of a binding site, wherein the binding site is capable of binding to a first antigen, optionally wherein the multimer is capable of binding to the first antigen and a second antigen, wherein the antigens are different.

For example, the multimer comprises from 4 to 32 (eg, from 4 to 24, or from 4 to 20, or from 4 to 16) copies of the binding site, ie, this means that the multimer does not comprise any more or less than said number. In an embodiment, the multimer comprises, 4, 5, 6, 7, 8, 9, 10, 11, 12, 13, 14, 15, 16, 17, 18, 19, 20, 21, 22, 23, 24, 25, 26, 27, 28, 29, 30, 31 or 32 copies of the binding site.

For example, the multimer contains from 4 to 32 (eg, from 4 to 24, or from 4 to 20, or from 4 to 16) copies of the binding site, ie, this means that the multimer does not have any more or less than said number. In an embodiment, the multimer contains, 4, 5, 6, 7, 8, 9, 10, 11, 12, 13, 14, 15, 16, 17, 18, 19, 20, 21, 22, 23, 24, 25, 26, 27, 28, 29, 30, 31 or 32 copies of the binding site. For example, the binding site is any binding site mentioned herein, for example, any VH, VL, VHH, dAb, nanobody, VH/VL pair, sybody or scFv.

In an embodiment, a control protein multimer comprising 1 or 2 (but no more than 1 or 2 respectively) of said binding sites is not capable of binding to the first antigen; or is capable of binding to the first antigen, but not to the second antigen. This is exemplified herein in Example 25. Binding may be determined by an ELISA assay, such as by determining OD₄₅₀, for example in an ELISA assay described herein. In an example, the first antigen is BCMA and the second antigen is TACI. Optionally, the antigens are human antigens. Optionally, the antigens are bacterial, archaeal or fungal antigens. Alternatively, the antigens are different viral antigens, or antigens of first and second viruses which viruses are different from each other, eg, SARS-CoV and SARS-CoV-2. Optionally, the virus antigens are spike proteins. Alternatively, the virus antigens are nucleocapsid (N) proteins. Alternatively, the virus antigens are envelope (E) proteins. Alternatively, the virus antigens are membrane (M) proteins. Optionally, the antigens of first and second viruses are different and the viruses are different strains of the same type of virus (eg, SARS-CoV strains; or SARS-CoV-2 strains; or influenza strains). Optionally, the antigens of first and second viruses are different and the viruses are different types of virus, eg, SARS-CoV and SARS-CoV-2.

The invention also provides multimers of binding sites that bind to virus antigens, as expemplified herein. Thus, the invention provides:

A protein multimer (first multimer) comprising more than 2 copies of a binding site, wherein the binding site is capable of binding to a virus protein (eg, a virus spike, E, M or N protein) of a first virus, optionally wherein the multimer is capable of binding to the first and a second virus, wherein the viruses are different. This is exemplified herein for 2 different viruses. For example, the multimer comprises 4 copies of the binding site, wherein the binding site is capable of binding to the virus protein (first virus protein) and a second virus protein which is a mutated version of the first virus protein, wherein the second virus protein is found in a second virus that is infectious to humans. For example, the first and second viruses are SARS viruses or coronaviruses, eg, SARS-CoV-2 viruses. For example, the first protein is a SARS-CoV-2 spike protein comprising the amino acid N501 (ie, asparagine at position 501) and the second protein is a a SARS-CoV-2 spike protein comprising the amino acid Y501 (ie, tyrosine at position 501) or T501. Additionally or alternatively, the first protein comprises E484 and the second protein comprises K484; and/or the first protein comprises K417 and the second protein comprises N417 or T417. Additionally or alternatively, compared to the first protein, the second protein comprises deletion ΔHV69-70, ΔY144 or ΔLLA242-244. Additionally or alternatively, the first protein comprises A222 and the second protein comprises V222; and/or the first protein comprises N439 and the second protein comprises K439; and/or the first protein comprises S477 and the second protein comprises N477; and/or the first protein comprises Y453 and the second protein comprises F453; and/or the first protein comprises F486 and the second protein comprises L486; and/or the first protein comprises G261 and the second protein comprises D261; and/or the first protein comprises V367 and the second protein comprises F367. Thus, we have discovered that multimers, as per the invention, comprising at least 4 copies of a binding site that binds to SARS-CoV-2 spike protein, such as the RBD (and preferably the inner face of the RBD) are particularly useful as medicaments (or diagnostic agents to idenfity the presence of the virus). As exemplified in Example 37, a multimer that recognises the epitope recognised by QB-GB binding site is capable of binding to several such mutant forms of SARS-CoV-2 spike and is well suited for administration to patients (or a human population) for treating or preventing SARS-CoV-2 infection, such as where some degree of resistance to mutants occurring during the life history of the virus is desired.

Optionally, each virus is a coronavirus. Optionally, one of the viruses is SARS-CoV and the other virus is SARS-Cov-2. For example, the first virus is SARS-CoV and the second virus is SARS-Cov-2. In an alternative, the first virus is SARS-CoV-2 and the second virus is SARS-Cov.

Optionally, the multimer comprises 4 copies of the binding site. Optionally, the multimer comprises 4 (but no more than 4) copies of the binding site. Optionally, the multimer comprises 8 (but no more than 8) copies of the binding site. Optionally, the multimer comprises 12 (but no more than 12) copies of the binding site. Optionally, the multimer comprises 16 (but no more than 16) copies of the binding site. Optionally, the multimer comprises 20 (but no more than 20) copies of the binding site. Optionally, the multimer comprises 24 (but no more than 24) copies of the binding site. Optionally, the multimer comprises 4, 8, 12, 16, 20 or 24 copies of the binding site.

Optionally,
(a) the binding of the multimer to the first antigen (or first virus protein) is stronger than the binding of a second multimer (eg, an immunoglobulin, such as an IgG) to the first antigen (or first virus protein), wherein the second multimer comprises 2 (but no more than 2) copies of said binding site; and/or
(b) the binding of the multimer to the second antigen (or a protein of the second virus, eg, a spike, E, M or N protein of the second virus) is stronger than the binding of a or said second multimer (eg, an immunoglobulin, such as an IgG) to the second antigen (or second virus protein), wherein the second multimer comprises 2 (but no more than 2) copies of said binding site.

Optionally,
(a) the binding of the multimer to the first virus spike protein is stronger than the binding of a second multimer (eg, an immunoglobulin, such as an IgG) to the first antigen (or first virus spike protein), wherein the second multimer comprises 2 (but no more than 2) copies of said binding site; and/or
(b) the binding of the multimer to a spike protein of the second virus is stronger than the binding of a or said second multimer (eg, an immunoglobulin, such as an IgG) to the second virus spike protein, wherein the second multimer comprises 2 (but no more than 2) copies of said binding site.

Optionally,
(a) the binding of the multimer to the first virus nucleocapsid (N) protein is stronger than the binding of a second multimer (eg, an immunoglobulin, such as an IgG) to the first virus N protein, wherein the second multimer comprises 2 (but no more than 2) copies of said binding site; and/or
(b) the binding of the multimer to a N protein of the second virus is stronger than the binding of a or said second multimer (eg, an immunoglobulin, such as an IgG) to the second virus N protein, wherein the second multimer comprises 2 (but no more than 2) copies of said binding site.

Optionally,
(a) the binding of the multimer to the first virus membrane (M) protein is stronger than the binding of a second multimer (eg, an immunoglobulin, such as an IgG) to the first virus M protein, wherein the second multimer comprises 2 (but no more than 2) copies of said binding site; and/or
(b) the binding of the multimer to a M protein of the second virus is stronger than the binding of a or said second multimer (eg, an immunoglobulin, such as an IgG) to the second virus M protein, wherein the second multimer comprises 2 (but no more than 2) copies of said binding site.

Optionally,
(a) the binding of the multimer to the first virus envelope (E) protein is stronger than the binding of a second multimer (eg, an immunoglobulin, such as an IgG) to the first virus E protein), wherein the second multimer comprises 2 (but no more than 2) copies of said binding site; and/or
(b) the binding of the multimer to a E protein of the second virus is stronger than the binding of a or said second multimer (eg, an immunoglobulin, such as an IgG) to the second virus E protein, wherein the second multimer comprises 2 (but no more than 2) copies of said binding site.

Preferably, binding or binding strength is determined by ELISA, eg, by determining OD₄₅₀. An ELISA herein may be carried out at room temperature and pressure (rtp), or preferably at 20 or 25 centigrade and 1 atmosphere.

Optionally,
(a) the first multimer binds to the first antigen (or first virus protein, eg, spike protein) with an OD₄₅₀ from 1 to 3 (such as from 1 to 2 or from 2 to 3) in an ELISA assay in which the first antigen or protein is at a concentration of 1 nM in the assay (and optionally the second multimer binds to the first antigen or protein with an OD₄₅₀ less than 0.5 in an ELISA assay in which the antigen or protein is at a concentration of 1 nM in the assay);
(b) the first multimer (i) binds to a first virus spike protein trimer with an OD₄₅₀ from 2 to 3 in an ELISA assay in which the spike protein is at a concentration of 1 nM in the assay and (ii) binds to a first virus spike protein monomer with an OD₄₅₀ from 1 to 2 in an ELISA assay in which the spike protein is at a concentration of 1 nM in the assay (optionally wherein the binding site comprises an ACE2 extracellular protein); and/or
(c) the first multimer binds to the first antigen (or a first virus protein) with an OD₄₅₀ from 2 to 3 (optionally from 2.5 to 3) in an ELISA assay in which the first antigen or protein is at a concentration of 10 nM in the assay (and optionally the second multimer binds to the first antigen or protein with an OD₄₅₀ from 1 to 2 in an ELISA assay in which the antigen or protein is at a concentration of 10 nM in the assay).

In an example, the multimer binds according to (a). In an example, the multimer binds according to (b). In an example, the multimer binds according to (c). In an example, the multimer binds according to (a) and (b). In an example, the multimer binds according to (a) and (c). In an example, the multimer binds according to (a), (b) and (c). In an example, the multimer binds according to (b) and (c). These are exemplified herein.

Binding of the the first multimer with said OD₄₅₀ indicates that the first multimer (ie, multimer of the invention) is useful for medical use, ie, therapy or prophylaxis of a disease or condition in a human or animal subject wherein the disease or condition is mediated by the first antigen (or a pathogen comprising the first antigen). Binding of the the second multimer (eg, IgG having only 2 of said binding sites) with said OD₄₅₀ indicates that the second multimer is not useful for medical use or said therapy or prophylaxis.

Binding of the the first multimer with said OD₄₅₀ indicates that the first multimer (ie, multimer of the invention) is useful for assaying for detecting the presence of the first antigen or antibodies against the first antigen in a bodily fluid sample of a human or animal, eg, a serum, saliva or cell sample obtained from a human or animal, wherein the human or animal (i) is suffering from, has suffered from or is suspected of suffering from a disease or conditionthat is mediated by the first antigen, or (ii) is suffering from, has suffered from or is suspected of suffering from an infection by a pathogen that comprises the first antigen, such as a virus, bacterium or fungus (eg, a yeast). Binding of the the second multimer (eg, IgG having only 2 of said binding sites) with said OD₄₅₀ indicates that the second multimer is not useful for such assaying or detection.

Generally, an Ig (eg, IgG) that binds to its cognate antigen with an affinity (Kd) higher than 1, 10, 100 or 1000 mM are not useful as medicaments. In an example, the binding site of the multimer of the invention is an antigen binding site of an Ig (eg, IgG) Fab fragment that binds to the antigen with an affinity (Kd) higher than 0.1, 1, 10, 100 or 1000 mM (eg, higher than 1 or 10 mM). Optionally, the Ig is said second multimer. In an example, the multimer of the invention binds to the antigen with an apparent affinity (avidity) of lower than 0.1 mM, 100 nM, 10 nM, 1 nM, 100 pM, 10 pM, 1 pM or 100 fM. These affinities are amenable to medical use. Affinities are may be determined by any standard method, for example by surface plasmon resonance (SPR) or ELISA, or bilayer interferometry (eg, as per the example below). The method may be carried out at rtp, or optionally at 20 or 25° C.entrigrade and 1 atm and optionally at a pH from 6.5 to 7.5 (eg, at pH 7). Reference is made to Science. 2020 May 8;368(6491):630-633. doi: 10.1 126/science.abb7269. Epub 2020 Apr 3, “A highly conserved cryptic epitope in the receptor binding domains of SARS-CoV-2 and SARS-CoV”, Yuan M et al, which determined binding affinity using a Fab version of CR3022, it can be seen CR3022 binds spike RBD of CoV-2 with at least 100x less affinity than CoV-1. The Kd for CR3022 for CoV-2 is around 115 nM. A multimer of the invention comprising 4 copies of the CR3022 binding site will have a Kd in the low pM range, thereby greatly improving on the apparent affinity and rendering the multimer useful as a medicament. We expect also a large improvement in affinity (expected to be in the double of single digit pM range or less) for the multimer binding to RBD of the CoV-1 strain. We would expect binding to such RBD with higher affinity again in the low pM range.

### Example Biolayer Interferometry Binding Assay

Binding assays may performed by biolayer interferometry (BLI) using an Octet Red® instrument (ForteBio). Briefly, His6-tagged antigen (eg, S or RBD protein) at 20 to 100 µg/mL in 1x kinetics buffer (1x PBS, pH 7.4, 0.01% BSA and 83 0.002% Tween 20) are loaded onto Anti-Penta-HIS™ (HIS1K) biosensors and incubated with the indicated concentrations of Fab or IgG (eg, CR3022 Fab or IgG) or multimer. The assay comprises fivesteps: 1) baseline: 60 s with 1x kinetics buffer; 2) loading: 300 s with his6-tagged proteins; 3) baseline: 60 s with 1x kinetics buffer; 4) association: 120 s with samples (Fab or IgG or multimer); and 5) dissociation: 120 s with 1x kinetics buffer. For estimating the exact Kd, a 1:1 binding model is used.

### Example ELISA Assay

ELISAs are performed in duplicates to compare the binding affinities of the different product formats. Recombinant antigen is diluted to 1 ug/ml in ELISA coating buffer (50 mM carbonate/bicarbonate). One hundred ul of 1 ug/ml antigen is added to each well of an ELISA plate and the plates are incubated overnight at 4° C. The plates are washed three times with PBS containing 0.05% Tween-20 before being blocked with 200 ul 1% bovine serum albumin in PBS for 4 hrs at room temperature. The plates are washed three times as before. Products (multimers or other protein to be tested) are serially diluted in PBS containing 0.05% Tween-20. One hundred ul of sample is added to each well and the plates are incubated overnight at 4° C. The plates are washed four times with PBS containing 0.05% Tween-20. One hundred ul of detection antibody (anti-His-HRP, A7058, Sigma; or antiHuman-IgG HRP, 31410, Thermo Fisher Scientific; or Protein L HRP, M00098, Genscript) diluted in blocking buffer (according to the manufacturers’ recommendations) is added to each well and the plates are incubated at room temperature for 2 h. Following four plate washes, 25 ul of TMB substrate solution (Thermo Fisher Scientific) is added to each well. The reaction is terminated after ~ 15 min by the addition of 25 ul 3 M HCl. The absorbance at 450 nm is read using a CLARIOstar™ microplate reader (BMG Labtech).

### Example SPR Binding Assay

The SPR is carried out at a detergent level of no greater than 0.05% by volume, eg, in the presence of P20 (polysorbate 20; eg, Tween-20™) at 0.05% and EDTA at 3 mM. In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022). In an example, the affinity (eg, of a VH/VL binding site) is determined using SPR by using any standard SPR apparatus, such as by Biacore™ or using the ProteOn XPR36™ (Bio-Rad®). The binding data can be fitted to 1:1 model inherent using standard techniques, eg, using a model inherent to the ProteOn XPR36™ analysis software.

Optionally,
(a) the first multimer binds to the first antigen (or first virus protein, eg, spike protein) with an OD₄₅₀ from 1 to 3 (such as from 1 to 2 or from 2 to 3) in an ELISA assay in which the antigen or protein is at a concentration of 1 nM in the assay (and optionally the second multimer binds to the first antigen or protein with an OD₄₅₀ less than 0.5 in an ELISA assay in which the antigen or protein is at a concentration of 1 nM in the assay);
(b) the first multimer (i) binds to a first virus spike protein trimer with an OD₄₅₀ from 2 to 3 in an ELISA assay in which the spike protein is at a concentration of 1 nM in the assay and (ii) binds to a first virus spike protein monomer with an OD₄₅₀ from 1 to 2 in an ELISA assay in which the spike protein is at a concentration of 1 nM in the assay (optionally wherein the binding site comprises an ACE2 extracellular protein); and/or
(c) the first multimer binds to the first antigen (or first virus protein, eg, spike protein) with an OD₄₅₀ from 2 to 3 (optionally from 2.5 to 3) in an ELISA assay in which the antigen or protein is at a concentration of 10 nM in the assay (and optionally the second multimer binds to the first antigen or protein with an OD₄₅₀ from 1 to 2 in an ELISA assay in which the antigen or protein is at a concentration of 10 nM in the assay).

In an example, the multimer binds according to (a). In an example, the multimer binds according to (b). In an example, the multimer binds according to (c). In an example, the multimer binds according to (a) and (b). In an example, the multimer binds according to (a) and (c). In an example, the multimer binds according to (a), (b) and (c). In an example, the multimer binds according to (b) and (c). These are exemplified herein.

Optionally, binding of the first multimer to the first antigen or protein is saturated as determined by OD₄₅₀ in an ELISA assay in which the antigen or protein is at a concentration between 10 and 100 nM in the assay (and optionally the second multimer binds to the first antigen or protein with an OD₄₅₀ less than 2.5 (eg, from 2 to 2.5) in an ELISA assay in which the antigen or protein is at a concentration between 10 and 100 nM in the assay).

Optionally,
(a) the first multimer binds to the second antigen (or second virus protein, eg, spike protein) with an OD₄₅₀ from 1 to 2 in an ELISA assay in which the second antigen or protein is at a concentration of 1 nM in the assay (and optionally the second multimer binds to the second antigen or protein with an OD₄₅₀ less than 0.5 in an ELISA assay in which the antigen or protein is at a concentration of 1 nM in the assay); and/or
(b) the first multimer binds to the second antigen (or second virus protein, eg, spike protein) with an OD₄₅₀ from 2 to 3 (optionally from 2.5 to 3) in an ELISA assay in which the second antigen or protein is at a concentration of 10 nM in the assay (and optionally the second multimer binds to the second antigen or protein with an OD₄₅₀ from 0.5 to 1.5 (eg, 0.5 to 1) in an ELISA assay in which the antigen or protein is at a concentration of 10 nM in the assay).

Optionally, binding of the first multimer to the second antigen or protein is saturated as determined by OD₄₅₀ in an ELISA assay in which the antigen or protein is at a concentration between 10 and 100 nM in the assay (and optionally the second multimer binds to the second antigen or protein with an OD₄₅₀ less than 1.5 (eg, from 1 to 1.5) in an ELISA assay in which the antigen or protein is at a concentration between 10 and 100 nM in the assay).

Optionally, the multimer is capable of detectably binding to antibodies that bind to the first antigen or the second antigen or virus protein (eg, anti-virus protein antibodies, such as anti-SARS-Cov spike antibodies or anti-SARS-Cov-2 spike antibodies or anti-influenza haemagglutinin antibodies) in an ELISA assay, wherein detection of the multimer binding is measured by OD₄₅₀ and the assay comprises
(a) Optionally diluting a serum sample of a mammal between 100 and 10⁶-fold;
(b) Contacting the antigen or protein (eg, SARS-Cov-2 spike protein) with the a serum sample of a mammal (which optionally has been diluted in step (a)) whereby anti-antigen or protein antibodies present in the sample bind to the antigen or protein, wherein the antigen or protein is immobilised on a solid surface;
(c) Contacting the bound antibodies with copies of the multimer; and
(d) Detecting multimer bound to antibody.

ELISA herein may be a sandwich ELISA.

Optionally, the dilution is from 10 to 10⁴, 10⁵ or 10⁶-fold. Optionally, the dilution is from 100 to 10⁴, 10⁵ or 10⁶-fold. Optionally, the dilution is from 1000 to 10⁴, 10⁵ or 10⁶-fold. Preferably, the dilution is 1000 to 1,000,000-fold (such as 1000 to 100,000-fold or 1000 to 10,000-fold). Optionally, dilution is dilution with water or an aqueous solution, eg, PBS, such as PBS containing from 0.1 to 0.05% (eg, either 0.1% or 0.05%) Tween-20. This is exemplified herein, demonstrating the possibility of extremely sensitive assaying using multimers of the invention comprising more than 2 (eg, at least 4) binding site copies.

Optionally, the spike protein is a trimer of polypeptides.

Optionally, the binding site is an antibody VH/VL pair or an antibody single variable domain (such as a nanobody, VHH or a dAb).

Optionally, the binding site is
(a) The spike protein binding site of an antibody selected from CR3022, CR3014, or any other anti-coronavirus antibody disclosed herein (eg, an antibody of Table 21);
(b) An ACE2 protein which is capable of binding to the first virus spike protein; or
(c) A TMPRSS2 protein which is capable of binding to the first virus spike protein.

Optionally, the binding site comprises or consists of an ACE2 extracellular protein. Optionally, the ACE2 protein is human ACE2 protein. For example, an extracellular protein of ACE2 having UNIPROT number Q9BYF1, the sequence of such ACE2 and the extracellular domain thereof being incorporated herein by reference, along with the nucleotide sequence encoding such. In an example, ACE2 extracellular protein comprises or consists of positions 18 to 615 or 18 to 740 of ACE2 having UNIPROT number Q9BYF1, the sequence comprising or consisting of positions 18 to 740 being incorporated herein by reference, along with the nucleotide sequence encoding such.

Optionally, the binding site comprises or consists of an TMPRSS2 extracellular protein. Optionally, the TMPRSS2 protein is human TMPRSS2 protein. For example, an extracellular protein of TMPRSS2 having UNIPROT number 015393, the sequence of such TMPRSS2 and the extracellular domain thereof being incorporated herein by reference, along with the nucleotide sequence encoding such. In an example, TMPRSS2 extracellular protein comprises or consists of positions 106 to 492 of TMPRSS2 having UNIPROT number 015393, the sequence comprising or consisting of positions 106 to 492 being incorporated herein by reference, along with the nucleotide sequence encoding such.

Optionally, the binding site is an antibody VH/VL pair, wherein the VH comprises an amino acid sequence of a VH disclosed in Table 23 and the VL comprises the amino acid sequence of the cognate VL disclosed in Table 23. Optionally, the binding site comprises an scFv disclosed in Table 23. Optionally, the binding site comprises an antibody single variable domain (eg, a VHH, nanobody, dAb, VH or VL) disclosed in Table 23, Table 32 or elsewhere herein.

Optionally, the binding site is an antibody VH/VL pair, wherein the VH comprises an amino acid sequence of a VH disclosed in Table 32 and the VL comprises the amino acid sequence of the cognate VL disclosed in Table 32. Optionally, the binding site comprises an scFv disclosed in Table 32.

Optionally, the multimer comprises a multimer of a polypeptide disclosed in Table 23, optionally wherein the polypeptide is a polypeptide in the Table that comprises a TD.

Optionally, the multimer comprises a multimer of a polypeptide disclosed in Table 32, optionally wherein the polypeptide is a polypeptide in the Table that comprises a TD.

Any amino acid sequence in Table 23, Table 32 or elsewhere herein that ends at its C-terminus in TVS may in the alternative be provided as the indentical sequence except that the alternative ends in TVSS. Any amino acid sequence in Table 23, Table 32 or elsewhere herein that ends at its C-terminus in TVSS may in the alternative be provided as the indentical sequence except that the alternative ends in TVS.

The multimer may be a multimer of any format disclosed herein. The multimer may be a multimer of any polypeptide dislosed herein.

Optionally, the multimer comprises more than 2 (eg, comprises 4) copies of a heavy/light chain pair, wherein each heavy chain comprises (in N- to C-terminal direction) a VH and an antibody constant region (eg, an Fc) and wherein each light chain comprises (in N- to C-terminal direction) a VL and an antibody constant region (eg, a CL), wherein the binding site of the multimer comprises the VH paired with the VL; optionally wherein each heavy chain comprises a self-assembly multimerization domain (such as a tetramerization domain, such as a p53 TD). Optionally, each heavy chain comprises a hinge region as disclosed herein.

Optionally, the multimer comprises more than 2 (eg, comprises 4) copies of a polypeptide, wherein the polypeptide comprises (in N- to C-terminal direction) a single variable domain and a multimerization domain (eg, a tetramerization domain, such as a p53 TD), and optionally an antibody constant region (eg, an Fc or CL) between the single variable domain and the multimerization domain, or the multimersiation domain is between the single variabl domain and the constant region.

In an aspect, the invention provides assays and methods:-
A method for detecting the presence of an antigen in a sample, the method comprising combining the sample with a multimer of the invention, allowing antigen in the sample to bind multimers to form antigen/multimer complexes and detecting antigen/multimer complexes.
The antigen may be a virus antigen, eg, a spike, M, E or N antigen, or a coronavirus antigen. The antigen may be comprised by an antibody present in the sample, eg, an antibody that is capable of binding do an antigen of an infectious disease pathogen (such as a virus or bacterium) or an antigen that is capable of binding to a human protein.
The sample may be a blood sample, serum sample, sputum sample, cell sample, saliva sample, bodily fluid sample of an animal or human subject. Optionally, the sample is diluted before said detection, eg, before said combining. Dilution may be any fold dilution disclosed herein, eg, and dilution by a PBS solution or water.
Optionally, the antigen is immobilised on a solid surface before or after said combining. Immobilisation may be carried out by binding the antigen to an anti-antigen immunoglobulin or superantigen that is bound to the solid surface. Examples of superantigens are Proteins A, L and G or antibody-binding fragments thereof. Immobilisation may be carried out by binding the antigen to a multimer of the invention that is bound to the solid surface, wherein the binding sites of the multimer are capable of binding to the antigen, eg wherein the antigen is comprised by an antibody. For example, the binding site is an antibody binding site of Protein G, A or L. Thus, in an example multimers of the invention are bound to a solid surface, the surface is contacted with the sample wherein antigen (eg, antibodies or virus particles) comprised by the sample are bound to multimers of the invention to form antigen/multimer complexes, and complexes are detected thereby determining the presence of the antigen in the sample. In a different example, the antigen is comprised by antibodies that are capable of binding to a second antigen (eg, a human, bacteria, fungal or viral protein, such as a viral spike, M, E or N protein), wherein the second antigen is immobilised on a solid surface, the surface is contacted with the sample, wherein antibodies comprised by the sample bind to the second antigen to form second antigen/antibody complexes, and complexes are contacted with multimers of the invention wherein multimers bind to antibodies whereby second antigen/antibody/multimer complexes are formed, and second antigen/antibody/multimer complexes are detected thereby determining the presence of said antibodies in the sample. For example, the antibodies are IgM, IgG, IgD, IgE or IgA antibodies, preferably IgM, IgG or IgA antibodies.
In an example, the method further comprises isolating complexes and optionally obtaining sequence information of antigen (first antigen, eg, antibodies) comprised by complexes. Optionally, the sequence may be inserted into an expression vector and expressed to produce proteins, eg, wherein the proteins are isolated. For example, VH and/or VL domain amino acid sequence of antibodies comprised by complexes is obtained and use to express copies of the VH and/or VL in an expression host, eg, CHO or HEK cell.

Optionally, the method is an ELISA method, eg, a sandwich ELISA. The method is carried out in vitro.

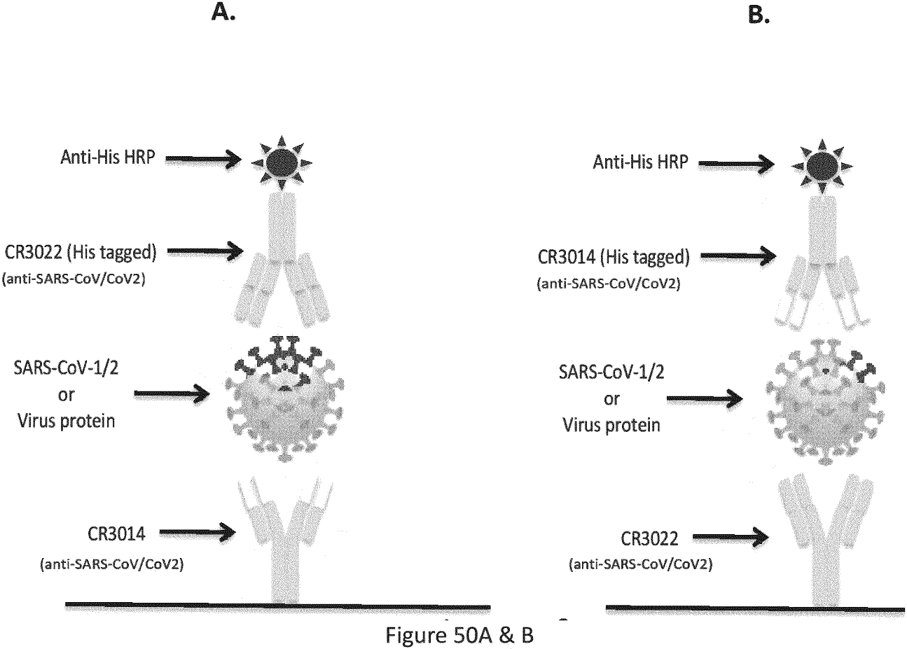

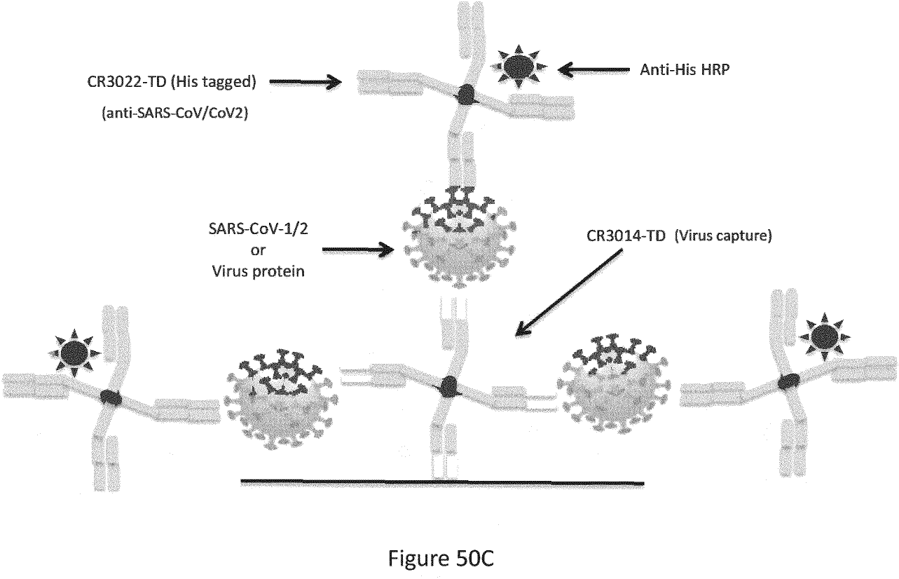

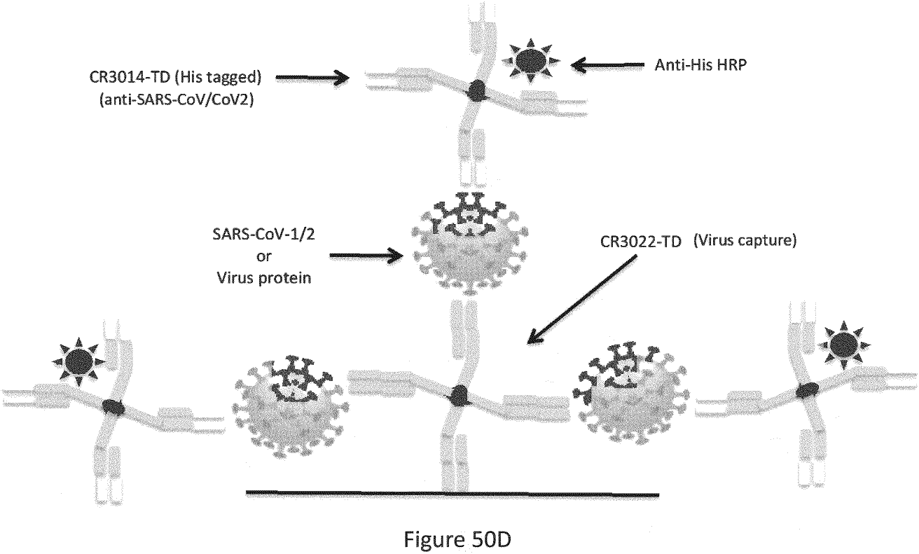

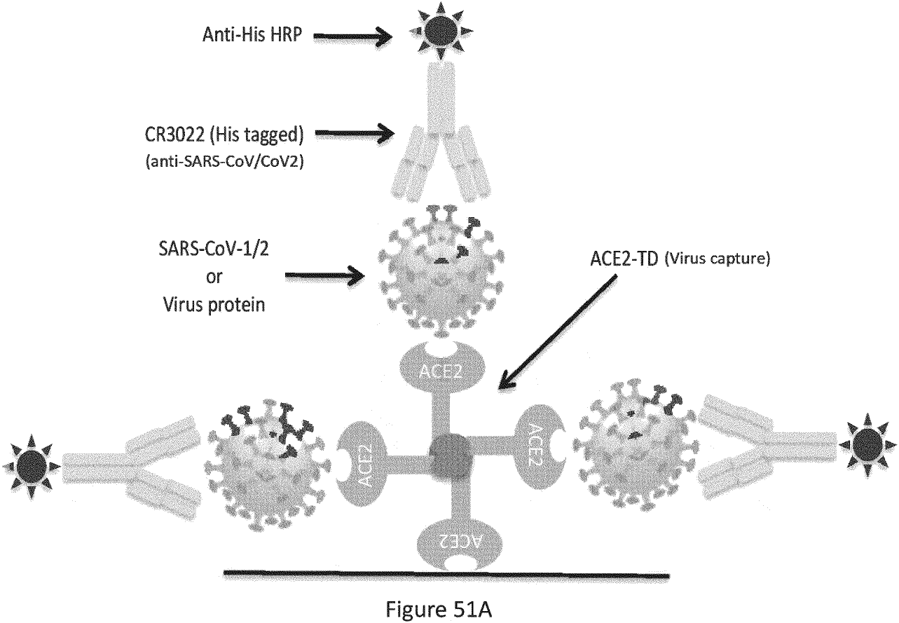

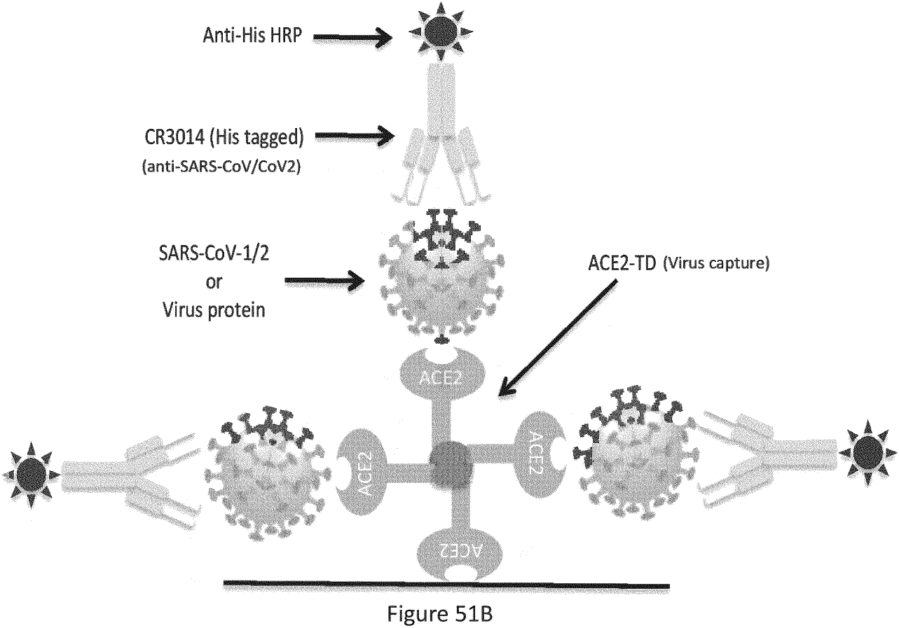

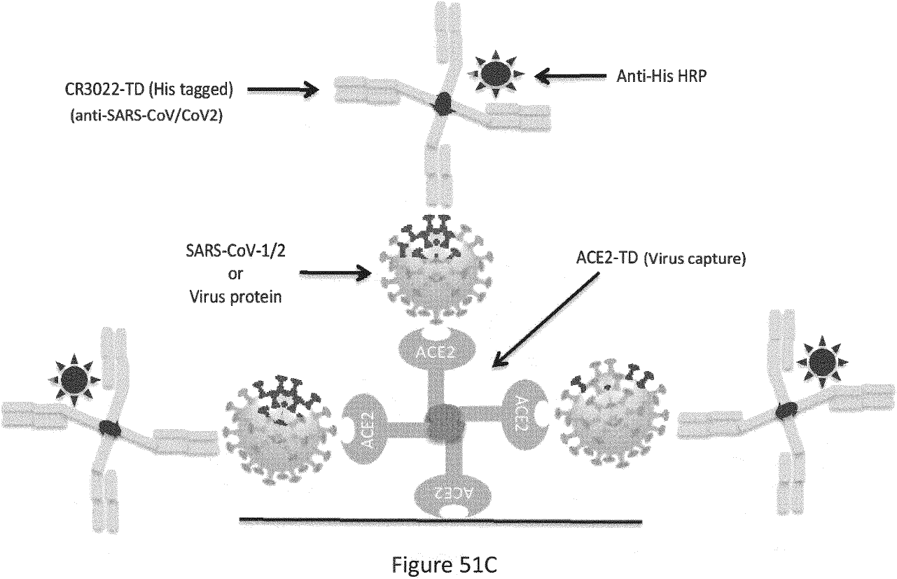

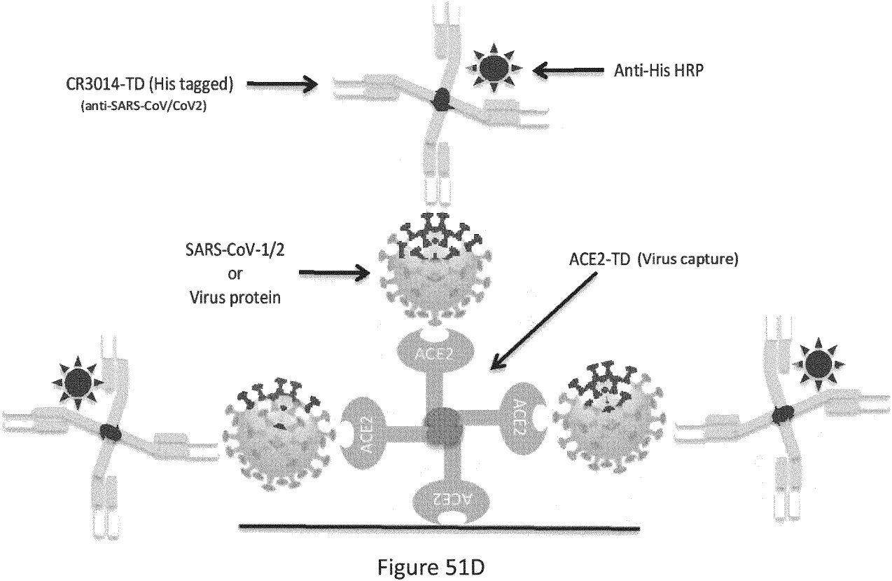

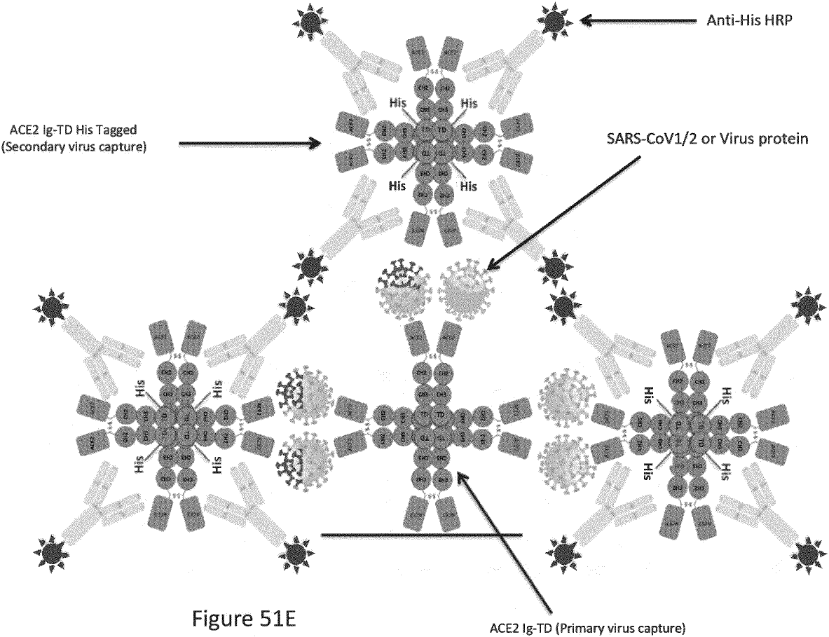

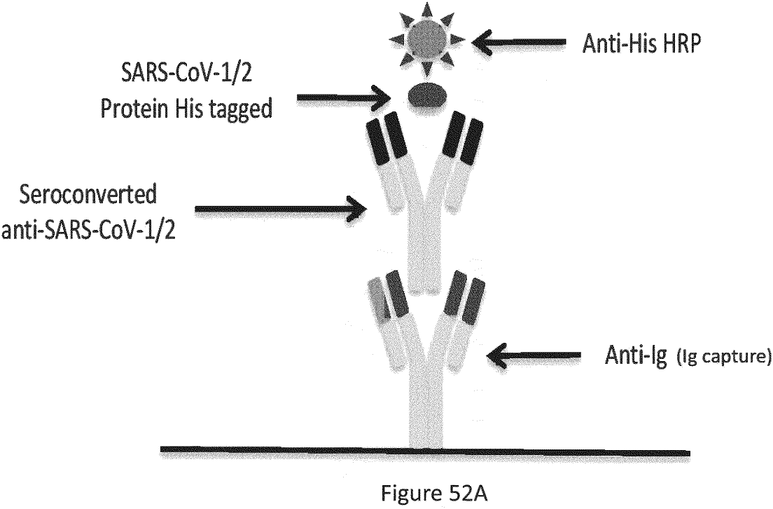

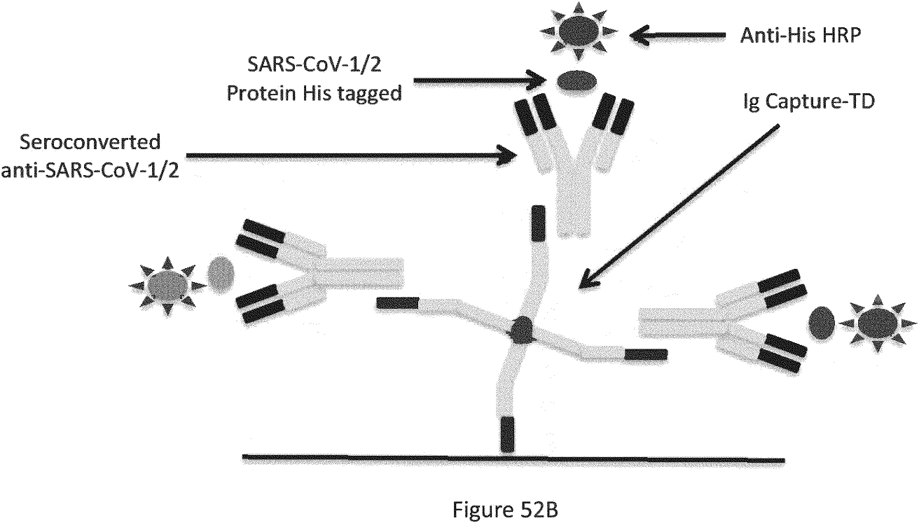

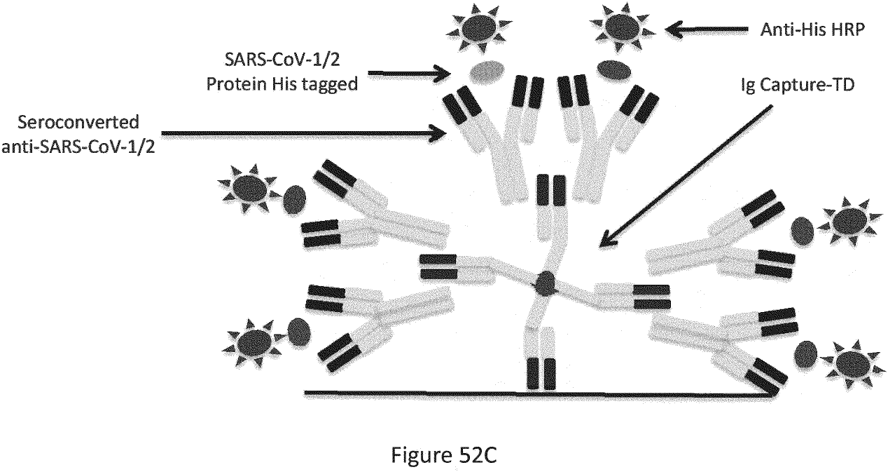

Suitable assay example formats are shown FIGS. 50-55, as well as reagents and polypeptides for making multimers of the invention that are useful for the methods and assays. The examples show assay formats relating to SARS-CoV and SARS-CoV2, but they are equally applicable mutatis mutandis to detecting pathogens (eg, any virus, bacterium or fungus) other than SARS-CoV and SARS-CoV2 and the disclosures of FIG. 50-55 can therefore in the alternative be read as relating to any pathogen, any pathogen antigen or protein, any anti-pathogen antibody and any other suitable multimer of the invention.

For example, the invention provides an assay comprising a format shown in any of FIGS. 50-55 for detecting the presence of a pathogen (eg, any virus, bacterium or fungus), anti-pathogen antibodies or a pathogen protein in a sample (eg, in serum, blood, saliva or any other sample disclosed herein). In an example, the pathogen is a coronavirus, eg, SARS-CoV or SARS-CoV-2, or infuenza virus or HIV, or any other virus disclosed herein.

In an embodiment, there is provided:-

A method of detecting the presence of anti-SARS-Cov-2 protein (eg, spike, M, E or N) antibodies in a serum sample, the method comprising carrying out an ELISA assay (eg, an assay disclosed herein), and the assay comprises
(a) Optionally diluting the serum sample from 10 to 10⁶-fold;
(b) contacting the SARS-Cov-2 protein with the serum sample (which optinally is diluted in step (a)) whereby anti-SARS-Cov-2 protein antibodies present in the sample bind to the virus protein to produce virus protein/antibody complexes; and
(c) contacting anti-SARS-Cov-2 virus protein antibodies with copies of the multimer of the invention; and
(d) detecting multimer bound to virus protein/antibody complexes, the detecting comprising detection of the multimer binding, optionally by determining optical density (eg, OD₄₅₀);
wherein the steps can be carried out in the order (a) (b) (c) and (d) or (a) (c) (b) and (d), or wherein steps (b) and (c) are carried out simultaneously and between steps (a) and (d).

The method is carried out in vitro. In an alternative, the antibodies are antibodies that bind to a protein of SARS-CoV or a different coronavirus. Optionally, the antibodies are antibodies that bind to a N, M or E proteins of a coronavirus, eg SARS-CoV or SARS-Cov-2.

Optionally, the presence of anti-antigen or protein antibodies (eg, anti-virus protein antibodies, such as anti-SARS-Cov-2 spike antibodies) in the sample is detected when the optical density (eg, OD₄₅₀) is greater than 0.1 or 0.5 (optionally, greater than 1, 1.5 or 2) in the assay.

Optionally wherein the spike protein is immobilised on a solid surface. Alternatively, the multimers are immobilised on a solid surface.

Optionally, the dilution is 1000 to 1,000,000-fold (such as 1000 to 100,000-fold or 1000 to 10,000-fold) or any other fold dilution disclosed herein. Optionally, the dilution is from 10 to 10⁴, 10⁵ or 10⁶-fold. Optionally, the dilution is from 100 to 10⁴, 10⁵ or 10⁶-fold. Optionally, the dilution is from 1000 to 10⁴, 10⁵ or 10⁶-fold. Optionally, dilution is dilution with water or an aqueous solution, eg, PBS, such as PBS containing from 0.1 to 0.05% (eg, either 0.1% or 0.05%) Tween-20.

Optionally, the spike protein is a trimer of polypeptides. For example, the spike protein is a monomer of either S1 or S2 spike ectodomain, a trimer of the spike, monomer of the spike receptor binding domain (RBD domain); or a RBD multimer, such as a dimer, trimer, tetramer or octamer of the RBD.

In an alternative to binding spike, the multimer of may bind a Nucleocapsid (N protein), membrane protein (M protein) or envelope protein (E protein) and the disclsoures herein referring to spike protein binding can apply mutatis mutandis to those alternatives.

Before carrying the method herein the serum sample may have been obtained by taking a blood sample or other bodily fluid sample from a mammal (eg, a human or animal, such as any animal disclosed herein). In an example, the human is a human suspected of having previously been infected or currently infected by a pathogen, eg a virus, bacterium or fungus comprising the antigen (first antigen), eg, SARS-CoV or SARS-Cov-2. For example, the human is a male, female, adult, teenager, child, baby or a human of at least 10, 20, 30, 40, 50, 60, 70 or 80 years’ of age (preferably over 50).

In embodiments, the binding site of the multimer is
(a) The spike protein binding site of an antibody, optionally an antibody selected from CR3022, CR3014, or any other anti-coronavirus antibody disclosed herein (eg, an antibody of Table 21);
(b) An ACE2 protein which is capable of binding to the spike protein; or
(c) A TMPRSS2 protein which is capable of binding to the spike protein.

Optionally, the multimer is a multimer of a polypeptide disclosed in Table 24, optionally wherein the polypeptide is a polypeptide in the Table that comprise a TD.

Optionally, the multimer comprises a plurality of copies of an Ig binding domain disclosed in Table 25, optionally wherein the multimer further comprises a plurality of copies of a further (ie, different) Ig binding domain disclosed in Table 25,

Optionally, the binding site of the multimer is alternatively capable of binding to an antibody (eg, an antibody that is capable of binding a human antigen, viral antigen, bacterial antigen or fungal antigen, such as an anti-SARS-Cov2 antibody, optionally wherein the binding site is comprised by
(a) Protein G or a fragment thereof;
(b) Protein A or a fragment thereof;
(c) Protein L or a fragment thereof; or
(d) An scFv or antibody single variable domain.

Optionally, step (c) is carried out before step (b), wherein the protein A, G, L or fragment, scFv or variable domain binding sites of the multimers bind a plurality of copies of the antibody (eg, anti-SARS-Cov2 antibody). For this option, the multimers may be immobilised on a solid support.

Optionally, the multimers are immobilised on a solid surface. Optionally, in any method or assay herein, the step of determining optical density (eg, OD₄₅₀) comprises labelling complexes comprising first antigen or protein (eg, spike protein) and multimers with horseradish peroxidase (HRP) and detecting the label (optionally at a wavelength of 450 nm). For example, the HRP is contacted with tetramethyl benzidine and abosorbance is read at 450 nm, whereby OD₄₅₀ is determined.

The invention also provides:-

A pharmaceutical composition or assay reagent comprising a plurality of multimers of the invention, optionally wherein the reagent comprises said multimers immobilised on a solid support.

In an example the following provide the solid support: Beads, petri dish, a laboratory apparatus, flow cell or a swab or dipstick. The support may be sterile or suitable for medical use.

For example, the pharmaceutical composition comprises a pharmaceutically-acceptable carrier, diluent or excipient.

The invention also provides:-

A multimer of the invention for administration to a human or animal subject for medical use.

A multimer of the invention for administration to a human or animal subject for treatment or prevention of an infection by a pathogen (eg, a virus, bacterium or fungus) that comprises the first antigen or protein, or a symptom of such an infection (eg, an unwanted inflammatory response).

A multimer of the invention for administration to a human or animal subject for treatment or prevention of an infection by the first and/or second virus, or a symptom of such an infection (eg, an unwanted inflammatory response).

A method of treating a disease, condition or symptom thereof in a human or animal subject, the method comprising administering to the subject a plurality of multimers of the invention. For example, the disease, condition or symptom is caused by the first antigen or protein (or by a pathogen that comprises the first antigen or protein, such as a virus that comprises the antigen or protein).

A method of treating a viral infection or symptom thereof in a human or animal subject, the method comprising administering to the subject a plurality of multimers of the invention.

The composition or multimers of the invention may be admistered in said use or method to the subject by any means, such as intravenously, orally, by inhalation or any other route disclosed herein.

The invention also provides:-

An assay kit comprising a reagent of the invention and an amount of the first antigen or protein (eg, viral spike protein), optionally wherein the reagent and protein are comprised by different containers.

### Expanding Utility of Binding Sites Through Multimerisation

Examples 23 -26 demonstrate how advantageously multimerization of the invention can repurpose a binding site which otherwise would not be useful or much less useful, such as for medical use (eg, for treatment or prophylaxis of a disease or condition mediated by or associated with an antigen to which the binding site binds), or for assay use (eg, detecting a pathogen or antigen that mediates, causes or is adversely associated with a a disease or condition in a subject). Through multimerization of the invention, very high-order multimers (eg, containing 8-24 copies of a binding site) can easily be achieved in a stable multimer that can be readily expressed, such as in eukaryotic expression systems and host cells (as demonstrated in the exemplification herein). The high-order multimers usefully can repurpose binding sites that individually have relatively low binding strength for an antigen, wherein in the multimers an avidity effect is produced rendering the combined binding strength of copies of the binding site well suited to medical applications or very sensitive assay detection of low levels of antigens in samples. Usefully, for example, we demonstrate this even for very diluted samples where the antigen is at very low concentration. This is advantageous, for example where the antigen is an antigen of a pathogen (eg, a virus, bacterium or fungus that causes disease, such as in humans, animals or plants); or where the antigen is comprised by antibodies produced by a human or animal subject in response to immunisation, such as in response to a pathogen or a human protein in the subject.

Thus, in an embodiment, the invention provides:-

A method of expanding a utility of an antigen (eg, a protein) binding site, the method comprising producing a multimer of the invention, wherein the multimer comprises a plurality of copies (eg, at least 4 or 8 copies) of the binding site.

In an example the utility is a medical utility, such as treating or preventing a disesase or condition mediated by the antigen in a human or animal subject (eg, an infection caused by a pathogen comprising the antigen). In an example the utility is an assay or detection method for determining the presence or relative amount of the antigen (or a pathogen comprising the antigen) or antibodies that bind the antigen in a sample (eg, an environmental sample or any sample of a human or animal subject disclosed herein). In an example, the method increases the sensitivity of assaying for the antigen or antibodies in a sample. For example, the sample is a blood or serum or saliva sample which has been diluted, such as diluted with fold dilution disclosed herein. For example, the utlity is a reduced propensity for producing false positive results in assaying for the presence of the antigen or antibodies that bind the antigen in a sample.

### Exemplary Polypeptides & Multimers

In examples, the invention provides a polypeptide comprising one or more copies of an antigen binding domain (BD), the polypeptide comprising or consisting of, in N- to C-terminal direction
(a) BD-TD;
(b) TD-BD;
(c) BD-BD-TD;
(d) TD-BD-BD
(e) BD-TD-BD-BD
(f) BD-BD-TD-BD
(g) BD-BD-TD-BD-BD.

Preferably in these examples, TD is a p53 TD, eg, a human p53TD.

Optionally, the BD is a single variable domain (also referred to as a domain antibody or dAb, eg, a nanobody or VHH, eg, a single variable domain comprising SEQ ID NO: 288, preferably Nb-112). Preferably, BD comprises the amino acid of QB-GB (SEQ ID NO: 307). Preferably, BD comprises the amino acid of QB-BG. Preferably, the BD comprises the amino acid of QB-FE. Preferably, BD comprises the amino acid of SEQ ID NO: 288. For example, the BD comprises an amino acid sequence that is at least 80, 85, 90, 95, 96, 97, 98 or 99% identical to SEQ ID NO: 288. Preferably, BD comprises the amino acid sequence of a VH or VL disclosed in Table 32 (optionally wherein in the multimer each said VH is paired with the cognate VL shown in Table 32; or optionally wherein in the multimer each said VL is paired with the cognate VH shown in Table 32, eg, the pair comprises the VH and VL of REGN10987, REGN10933 or CB6). Preferably, BD comprises the VH or VL of REGN10987 (optionally wherein in the multimer each said VH is paired with the VL of REGN10987; or optionally wherein in the multimer each said VL is paired with the VH of REGN10987). Preferably, BD comprises the VH or VL of REGN10933 (optionally wherein in the multimer each said VH is paired with the VL of REGN10933; or optionally wherein in the multimer each said VL is paired with the VH of REGN10933). Preferably, BD comprises the VH or VL of CB6 (optionally wherein in the multimer each said VH is paired with the VL of CB6; or optionally wherein in the multimer each said VL is paired with the VH of CB6). Antibody CB6 is also known as LY-CoV555.

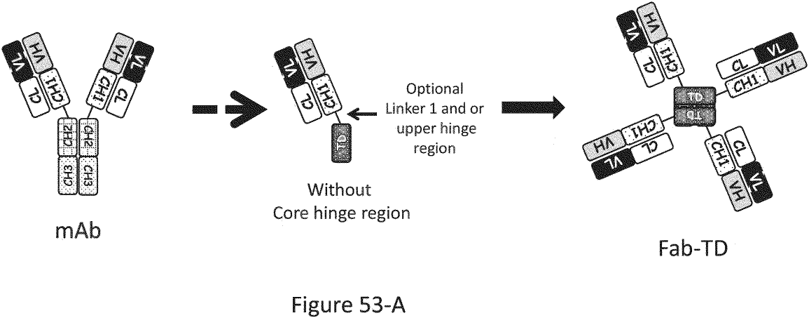

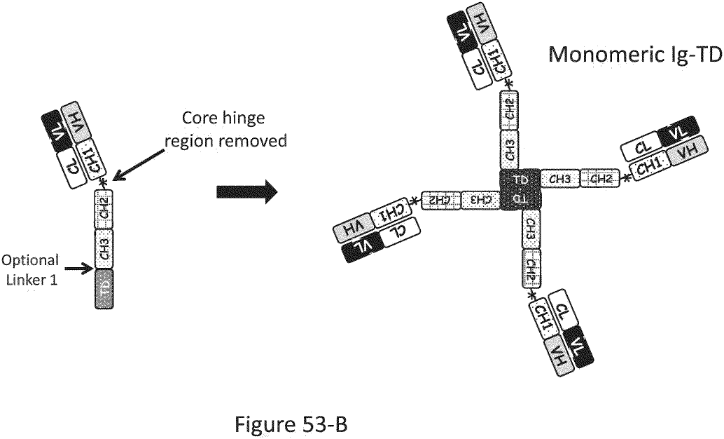

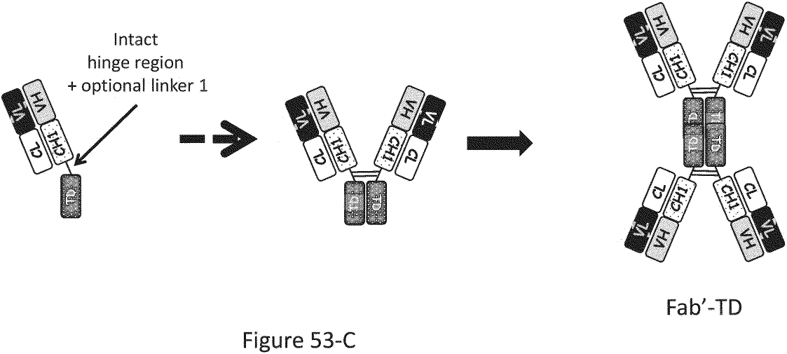

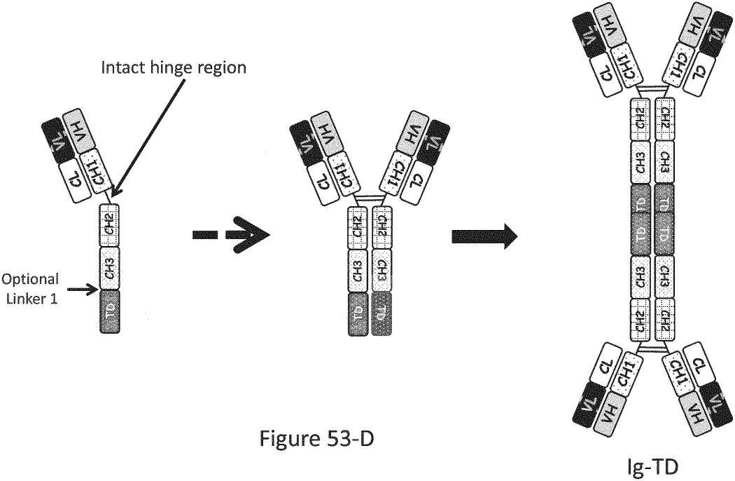

Optionally, example (a) is BD-CH1-TD, where BD= an antibody VH domain and CH1 is an antibody CH1 domain. In an embodiment, this polypeptide is paired with a second polypeptide comprising or consisting of, in N- to C-terminal direction BD2-CL, wherein BD2=an antibody VL domain, wherein the VH and VL form an antigen binding site and the CH1 pairs with the CL. An optional peptide linker may be between the TD and a domain (eg, the CH1) that is immediately N-terminal to the TD in the polypeptide. Multimerisation of 4 copies of the polypeptide using TDs produces a multimer (ie, tetramer) comprising 4 identical antigen binding sites, see, eg, FIG. 53A. The invention provides such a multimer.

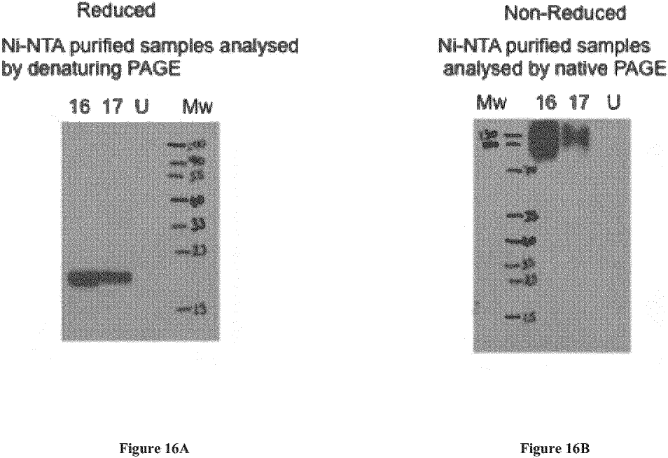

For example, in the immediately preceding paragraph BD and BD2 respectively comprise the VH and VL of an antibody selected from REGN10987, REGN10933 and CB6 (see Table 32 for sequences). For example, the multimer comprises the monomer (middle schematic) shown in any of FIGS. 16-A to 16-C. For example, in the immediately preceding paragraph BD and BD2 respectively comprise the VH and VL of an antibody selected from REGN10987, REGN10933, CB6, rRBD-15 (ABLINK Biotech Co., Ltd / Chengdu Medical College), B38, H4 (Capital Medical University, Beijing), FYB-207 (Formycon AG), ABP300 (Abpro Corporation), BRII-198 (Brii Biosciences, TSB Therapeutics (Beijing) CO.LTD), BRII-196 (Brii Biosciences, TSB Therapeutics (Beijing) CO.LTD), CT-P59 (Celltrion), HFB-3013, or HFB30132A (HiFiBiO Therapeutics), MW33 (Mabwell), SAB-185 (SAB Biotherapeutics), Etesevimab (Junshi Biosciences), SCTA01 or H014 (University of Chinese Academy of Sciences), STI-1499 or COVI-GUARD (Sorrento Therapeutics), TY027 (Tychan), COVI-AMG™ or STI-2020 (Sorrento Therapeutics), HLX70 (Hengenix Biotech Inc), ADM03820 (Ology Bioservices), an antibody comprised by XAV-19 (Nantes University Hospital), BGB DXP-593 or DXP-604 (BeiGene), VIR-7831 or GSK4182136 (Vir Biotechnology, GSK), AZD8895 or AZD1061 (AstraZeneca), HBM9022 or 47D11 (AbbVie, Harbour BioMed, Utrecht University and Erasmus Medical Center), Ab8 (University of Pittsburgh), MAbCo19 (AchilleS Vaccines Srl), AR-701 or AR-711 (Aridis Pharmaceuticals), DXP-604 (BeiGene), Centi-B9 (Centivax), GIGA-2050 (GigaGen), TATX-03 or TATX-06 or TATX-09 or TATX-13 or TATX-16 (ImmunoPrecise Antibodies), MTX-COVAB (Memo Therapeutics), NOVOAB-20 (NovoAb), COVI-SHIELD (Sorrento Therapeutics), STI-4920 or ACE-MAB or CMAB020 (MabPharm), IDB003 (IDBiologics) and VIR-7832 (Vir Biotechnology, GSK).

For example, the effector domain or binding domain or binding site of a polypeptide herein comprises the VH and/or VL of an antibody selected from REGN10987, REGN10933, CB6, rRBD-15 (ABLINK Biotech Co., Ltd / Chengdu Medical College), B38, H4 (Capital Medical University, Beijing), FYB-207 (Formycon AG), ABP300 (Abpro Corporation), BRII-198 (Brii Biosciences, TSB Therapeutics (Beijing) CO.LTD), BRII-196 (Brii Biosciences, TSB Therapeutics (Beijing) CO.LTD), CT-P59 (Celltrion), HFB-3013, or HFB30132A (HiFiBiO Therapeutics), MW33 (Mabwell), SAB-185 (SAB Biotherapeutics), Etesevimab (Junshi Biosciences), SCTA01 or H014 (University of Chinese Academy of Sciences), STI-1499 or COVI-GUARD (Sorrento Therapeutics), TY027 (Tychan), COVI-AMG™ or STI-2020 (Sorrento Therapeutics), HLX70 (Hengenix Biotech Inc), ADM03820 (Ology Bioservices), an antibody comprised by XAV-19 (Nantes University Hospital), BGB DXP-593 or DXP-604 (BeiGene), VIR-7831 or GSK4182136 (Vir Biotechnology, GSK), AZD8895 or AZD1061 (AstraZeneca), HBM9022 or 47D11 (AbbVie, Harbour BioMed, Utrecht University and Erasmus Medical Center), Ab8 (University of Pittsburgh), MAbCo19 (AchilleS Vaccines Srl), AR-701 or AR-711 (Aridis Pharmaceuticals), DXP-604 (BeiGene), Centi-B9 (Centivax), GIGA-2050 (GigaGen), TATX-03 or TATX-06 or TATX-09 or TATX-13 or TATX-16 (ImmunoPrecise Antibodies), MTX-COVAB (Memo Therapeutics), NOVOAB-20 (NovoAb), COVI-SHIELD (Sorrento Therapeutics), STI-4920 or ACE-MAB or CMAB020 (MabPharm), IDB003 (IDBiologics) and VIR-7832 (Vir Biotechnology, GSK).

For example, the multimer herein comprises at least 4 copies (eg, 4, 8, 12, 16, 20, 24 or 28 copies) of the VH and/or VL of an antibody selected from REGN10987, REGN10933, CB6, rRBD-15 (ABLINK Biotech Co., Ltd / Chengdu Medical College), B38, H4 (Capital Medical University, Beijing), FYB-207 (Formycon AG), ABP300 (Abpro Corporation), BRII-198 (Brii Biosciences, TSB Therapeutics (Beijing) CO.LTD), BRII-196 (Brii Biosciences, TSB Therapeutics (Beijing) CO.LTD), CT-P59 (Celltrion), HFB-3013, or HFB30132A (HiFiBiO Therapeutics), MW33 (Mabwell), SAB-185 (SAB Biotherapeutics), Etesevimab (Junshi Biosciences), SCTA01 or H014 (University of Chinese Academy of Sciences), STI-1499 or COVI-GUARD (Sorrento Therapeutics), TY027 (Tychan), COVI-AMG™ or STI-2020 (Sorrento Therapeutics), HLX70 (Hengenix Biotech Inc), ADM03820 (Ology Bioservices), an antibody comprised by XAV-19 (Nantes University Hospital), BGB DXP-593 or DXP-604 (BeiGene), VIR-7831 or GSK4182136 (Vir Biotechnology, GSK), AZD8895 or AZD1061 (AstraZeneca), HBM9022 or 47D11 (AbbVie, Harbour BioMed, Utrecht University and Erasmus Medical Center), Ab8 (University of Pittsburgh), MAbCo19 (AchilleS Vaccines Srl), AR-701 or AR-711 (Aridis Pharmaceuticals), DXP-604 (BeiGene), Centi-B9 (Centivax), GIGA-2050 (GigaGen), TATX-03 or TATX-06 or TATX-09 or TATX-13 or TATX-16 (ImmunoPrecise Antibodies), MTX-COVAB (Memo Therapeutics), NOVOAB-20 (NovoAb), COVI-SHIELD (Sorrento Therapeutics), STI-4920 or ACE-MAB or CMAB020 (MabPharm), IDB003 (IDBiologics) and VIR-7832 (Vir Biotechnology, GSK). The multimer may comprise no more than said number of copies.

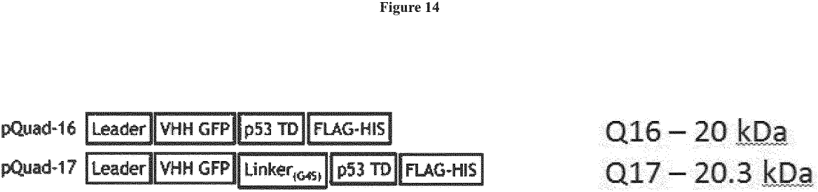

In an example, there is a provided a tetramer of an antibody (or a fragment of an antibody, eg, a Fab of an antibody), wherein the tetramer is tetramersised using tetramerization domains (TDs). For example, the tetramer has the configuration shown in the right-hand-side schematic of any one of FIGS. 14C, 14-D, 15-I, 15-J and 16-A to 16-C(and there may further be other moieties, such as one or more additional peptides, domains or proteins comprised by the tetramer that are not shown in said Figure). In an example, there is provided an antibody (or a fragment of an antibody, eg, a Fab of an antibody), wherein the antibody or fragment comprises a TD (eg, a p53 TD), preferably wherein the TD is at the N-terminus of at least one of the polypeptide chains of the antibody or fragment. For example, one or both of the heavy chains of the antibody or fragment comprise a TD at its N-terminus. For example, one or both of the light chains of the antibody or fragment comprise a TD at its N-terminus. For example, the antibody or fragment has the configuration shown in the middle schematic of any one of FIGS. 14C, 14-D, 15-I, 15-J and 16-A to 16-C (and there may further be other moieties, such as one or more additional peptides, domains or proteins comprised by the antibody or fragment that are not shown in said Figure). In an embodiment, the antibody is an antibody disclosed in the immediately preceding paragraph. In an embodiment, there is provided a tetramer of said antibody or fragment that comprises a TD.

A multimer or tetramer herein may have the configuration shown in the any one of the Figures herein (and there may further be other moieties, such as one or more additional peptides, domains or proteins comprised by the multimer or tetramer that are not shown in said Figure). A multimer or tetramer herein may have the configuration shown in the right-hand-side schematic of any one of FIGS. 14A to 14-F, 15-A to 15-L and 16-A to 16-C(and there may further be other moieties, such as one or more additional peptides, domains or proteins comprised by the multimer or tetramer that are not shown in said Figure). A polypeptide or monomer herein may have the configuration shown in any one of the Figures herein (and there may further be other moieties, such as one or more additional peptides, domains or proteins comprised by the polypeptide or monomer that are not shown in said Figure). A polypeptide or monomer herein may have the configuration shown in the middle schematic of any one of FIGS. 14A to 14-F, 15-A to 15-L and 16-A to 16-C (and there may further be other moieties, such as one or more additional peptides, domains or proteins comprised by the polypeptide or monomer that are not shown in said Figure). In the multimer or tetramer the multimer the multimer or tetramer may comprise 4 VH/VL pairs, such as shown in the right-hand-side schematic of any one of FIGS. 14A to 14-F, 15-A to 15-L and 16-A to 16-C. For example, each of said VH/VL pairs is a VH/VL antigen binding site comprised by an antibody disclosed herein eg, any antibody selected from REGN10987, REGN10933, CB6, rRBD-15 (ABLINK Biotech Co., Ltd / Chengdu Medical College), B38, H4 (Capital Medical University, Beijing), FYB-207 (Formycon AG), ABP300 (Abpro Corporation), BRII-198 (Brii Biosciences, TSB Therapeutics (Beijing) CO.LTD), BRII-196 (Brii Biosciences, TSB Therapeutics (Beijing) CO.LTD), CT-P59 (Celltrion), HFB-3013, or HFB30132A (HiFiBiO Therapeutics), MW33 (Mabwell), SAB-185 (SAB Biotherapeutics), Etesevimab (Junshi Biosciences), SCTA01 or H014 (University of Chinese Academy of Sciences), STI-1499 or COVI-GUARD (Sorrento Therapeutics), TY027 (Tychan), COVI-AMG™ or STI-2020 (Sorrento Therapeutics), HLX70 (Hengenix Biotech Inc), ADM03820 (Ology Bioservices), an antibody comprised by XAV-19 (Nantes University Hospital), BGB DXP-593 or DXP-604 (BeiGene), VIR-7831 or GSK4182136 (Vir Biotechnology, GSK), AZD8895 or AZD1061 (AstraZeneca), HBM9022 or 47D11(AbbVie, Harbour BioMed, Utrecht University and Erasmus Medical Center), Ab8 (University of Pittsburgh), MAbCo19 (AchilleS Vaccines Srl), AR-701 or AR-711 (Aridis Pharmaceuticals), DXP-604 (BeiGene), Centi-B9 (Centivax), GIGA-2050 (GigaGen), TATX-03 or TATX-06 or TATX-09 or TATX-13 or TATX-16 (ImmunoPrecise Antibodies), MTX-COVAB (Memo Therapeutics), NOVOAB-20 (NovoAb), COVI-SHIELD (Sorrento Therapeutics), STI-4920 or ACE-MAB or CMAB020 (MabPharm), IDB003 (IDBiologics) and VIR-7832 (Vir Biotechnology, GSK).

There is provided a mixture of at least 2 (eg, 2 or 3) different multimers, wherein each multimer is according to the invention. For example, a first of said multimers comprises 4 copies of an antigen binding site that is capable of binding to a first antigen; and a second of said multimers comprises 4 copies of an antigen binding site that is capable of binding to a second antigen, optionally the antigens are identical and the binding sites bind different epitopes comprised by the antigen, or the antigens are different. For example, the or each antigen is an antigen of a virus, eg, SARS-CoV or SARS-Cov-2 antigen, such as spike antigen, or the virus is influenza virus or any other virus disclosed herein. For example, wherein a first of said multimers comprises 4 copies of the SARS-CoV-2 antigen binding site of REGN10987, and a second of said multimers comprises 4 copies of the SARS-CoV-2 antigen binding site of REGN10933.

For example, the binding site (eg, BD or BD2) of a polypeptide or multimer herein comprises the variable domain of Nb11-59 (Shanghai Novamab Biopharmaceuticals Co., Ltd.), MERS VHH-55, SARS VHH-72 or VHH303 72-Fc. For example, the binding site (eg, BD or BD2) of a polypeptide or multimer herein comprises a Darpin of MP0420 or MP0423.

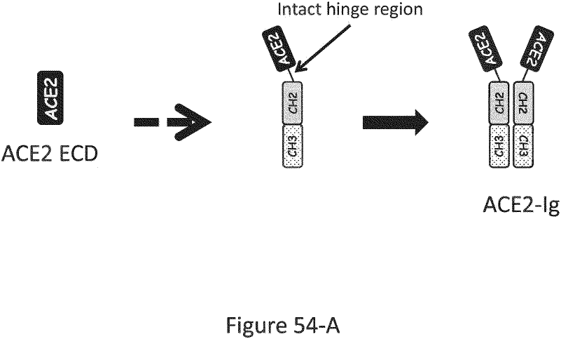

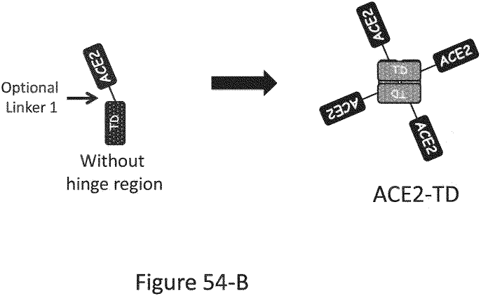

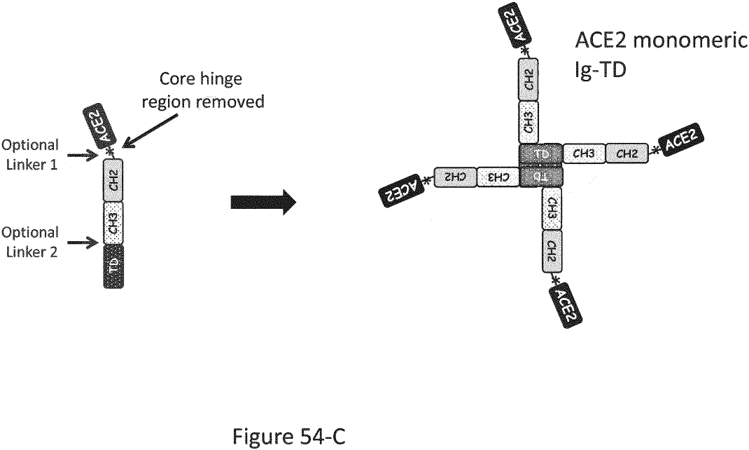

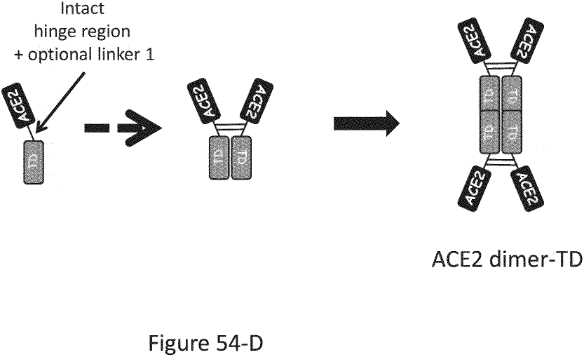

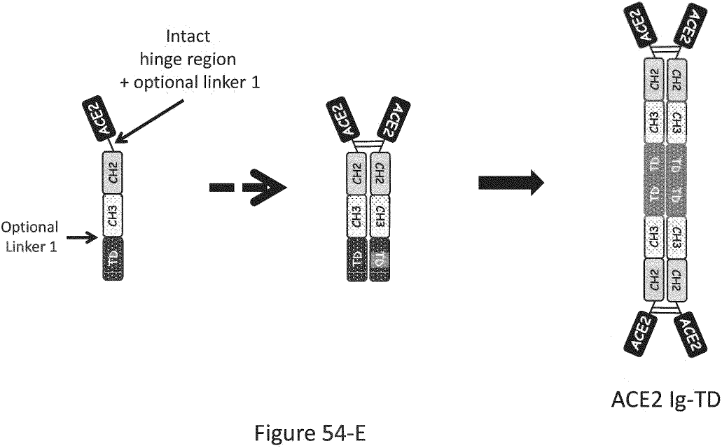

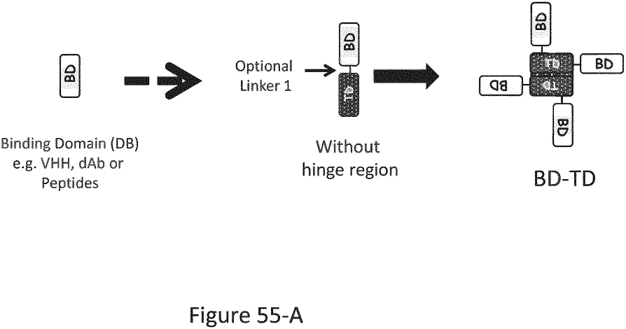

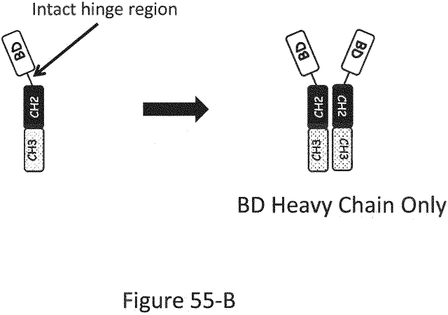

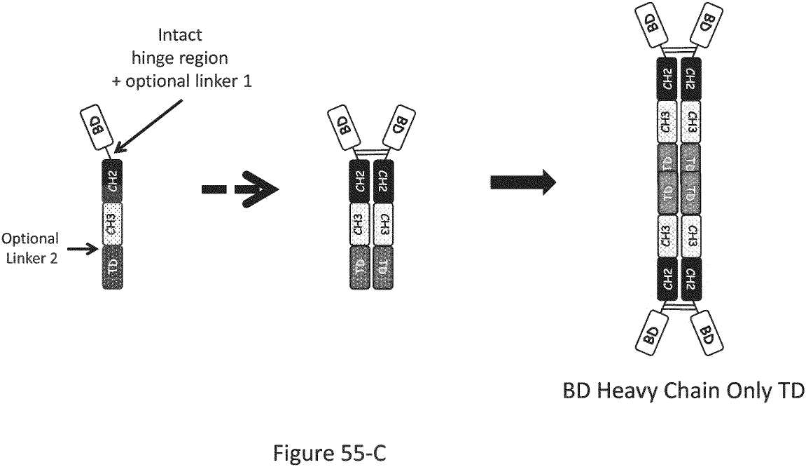

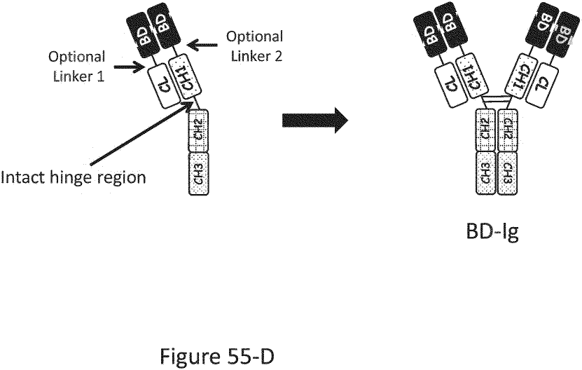

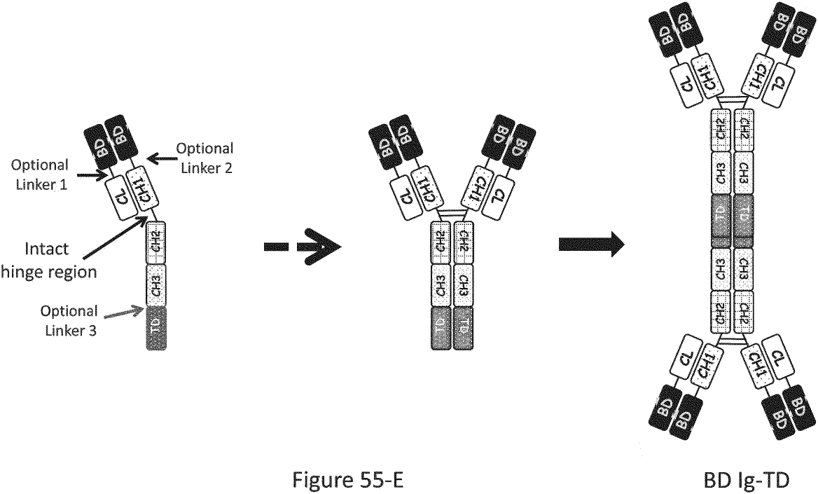

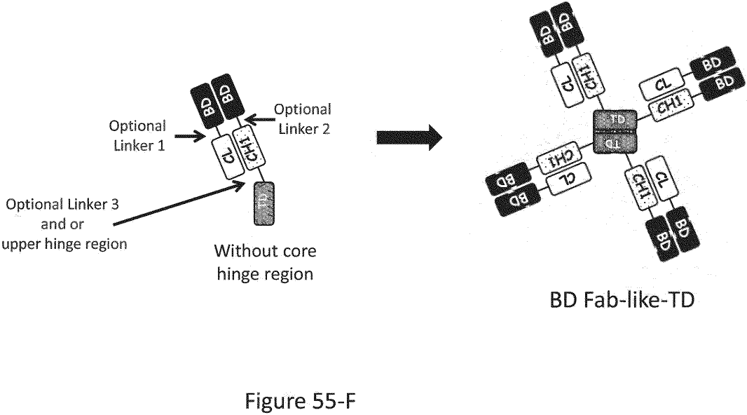

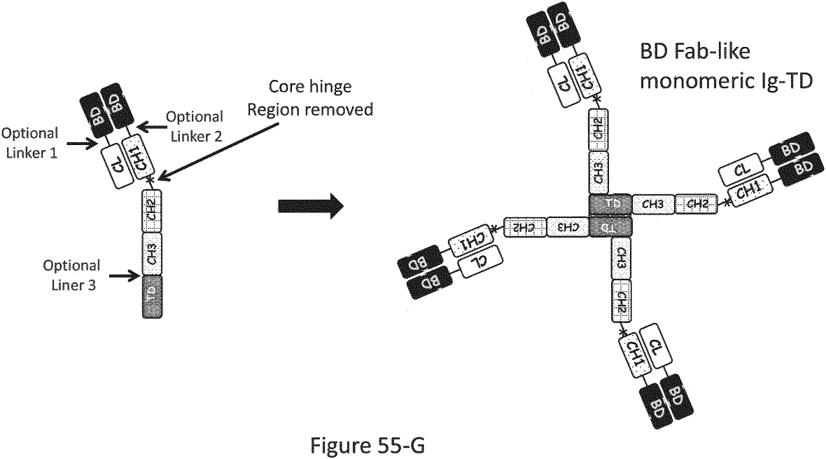

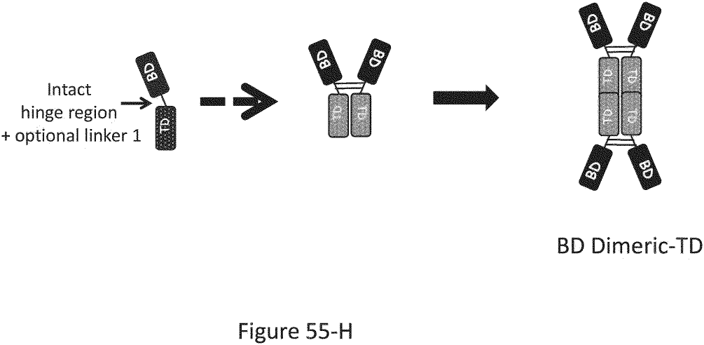

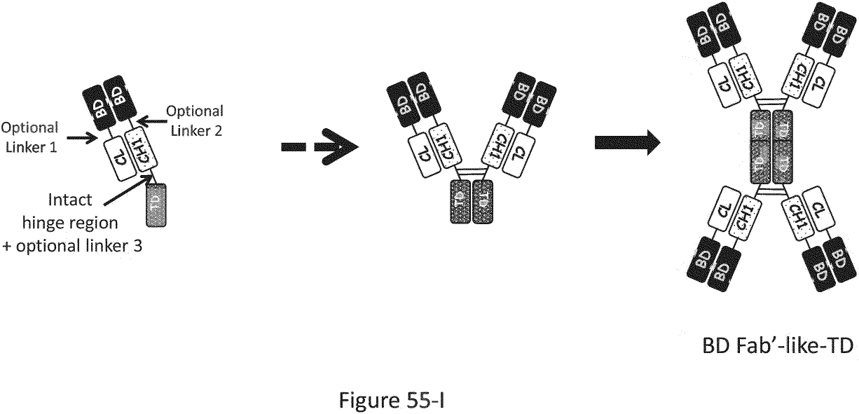

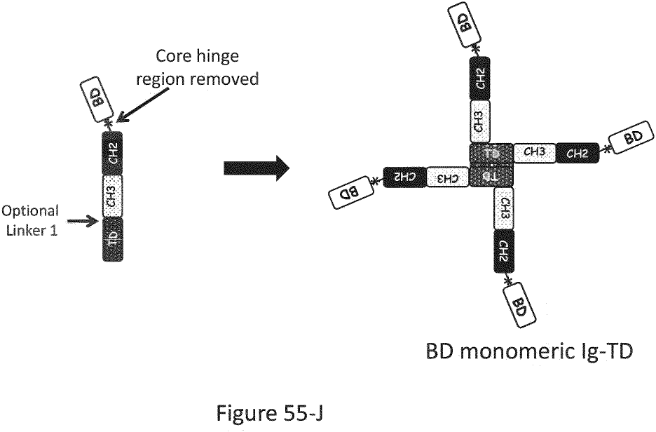

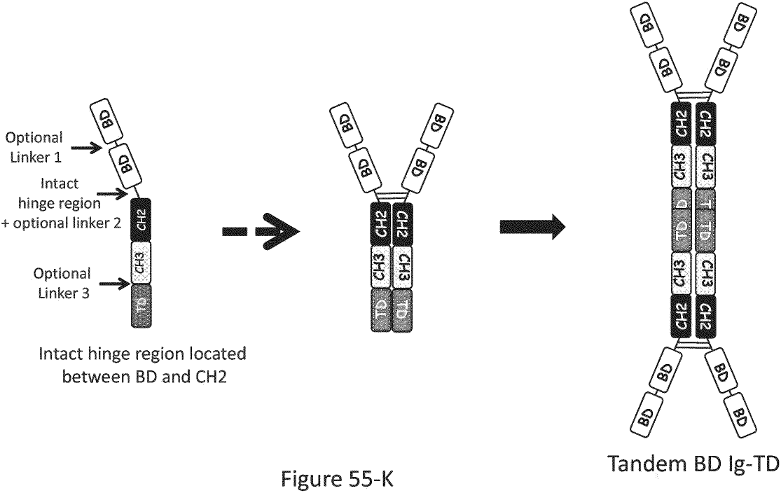

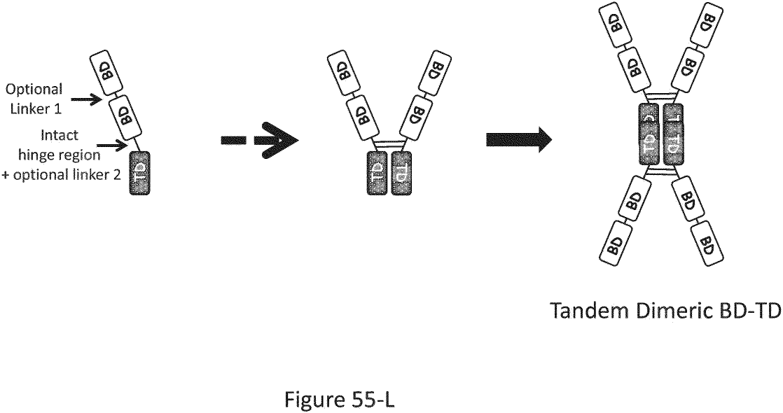

Optionally, example (a) is BD-CH1-hinge-Fc-TD, where BD= an antibody VH domain and CH1 is an antibody CH1 domain, optionally the hinge is devoid of a core hinge region or is any other hinge disclosed herein and Fc is an antibody Fc region (ie, CH2-CH3). In an embodiment, this polypeptide is paired with a second polypeptide comprising or consisting of, in N- to C-terminal direction BD2-CL, wherein BD2=an antibody VL domain, wherein the VH and VL form an antigen binding site and the CH1 pairs with the CL. An optional peptide linker may be between the TD and a domain (eg, the CH3) that is immediately N-terminal to the TD in the polypeptide. Multimerisation of 4 copies of the polypeptide using TDs produces a multimer (ie, tetramer) comprising 4 identical antigen binding sites, see, eg, FIG. 53C. The invention provides such a multimer. In an alternative, example (a) is BD-Fc-TD (eg, see FIGS. 54C, 55C) and multimers thereof are also provided by the invention, such as tetramers of such polypeptide.

In an example, the invention provides a polypeptide comprising an antigen binding domain (BD), the polypeptide comprising or consisting of, in N- to C-terminal direction BD-Fc-Td, wherein Fc is an antibody Fc region. In an embodiment, there is provided a dimer of first and second copies of such a polypeptide, wherein the Fc of the first polypeptide is associated with the Fc of the second polypeptide. In an embodiment there is a dimer of such a dimer, eg, as shown in FIG. 12C. Optionally, the BD is a single variable domain (also referred to as a domain antibody or dAb, eg, a nanobody or VHH). Optionally in such a polypeptide, dimer or tetramer BD comprises the amino acid of QB-GB (SEQ ID NO: 307), QB-DD, QB-BG or QB-FE (see Table 23 for sequences).

In an example, the invention provides a provides polypeptide comprising an antigen binding domain (BD), the polypeptide comprising or consisting of, in N- to C-terminal direction BD-CH1-Fc-Td, wherein Fc is an antibody Fc region. In an embodiment, there is provided a dimer of first and second copies of such a polypeptide, wherein the Fc of the first polypeptide is associated with the Fc of the second polypeptide. In an embodiment there is a dimer of such a dimer, eg, as shown in FIG. 12E Preferably each polypeptide is paired with a further polypeptide, wherein the further polypeptide comprises, in N- to C-terminal direction, BD2-CL, wherein the CH1pairs with the CL. Optionally, the BD is a single variable domain (also referred to as a domain antibody or dAb, eg, a nanobody or VHH). Optionally, the BD2 is a single variable domain (also referred to as a domain antibody or dAb, eg, a nanobody or VHH). In an alternative, BD and BD2 are a VH/VL pair that binds an antigen. The CL of said further polypeptide associates with the CH1 of the other polypeptide (see, eg, FIG. 12E). Optionally in such a polypeptide, dimer or tetramer BD comprises the amino acid of QB-GB (SEQ ID NO: 307), QB-BG or QB-FE (see Table 23 for sequences).

In an example, the invention provides a provides polypeptide comprising an antigen binding domain (BD), the polypeptide comprising or consisting of, in N- to C-terminal direction BD-CH1-Td, wherein CH1 is an antibody CH1. In an embodiment, there is provided a dimer of first and second copies of such a polypeptide, eg, wherein the TDs of the polypeptides are associated together. In an embodiment there is a dimer of such a dimer, eg, as shown in FIG. 12I Preferably each polypeptide is paired with a further polypeptide, wherein the further polypeptide comprises, in N- to C-terminal direction, BD2-CL, wherein the CH1 pairs with the CL. Optionally, the BD is a single variable domain (also referred to as a domain antibody or dAb, eg, a nanobody or VHH). Optionally, the BD2 is a single variable domain (also referred to as a domain antibody or dAb, eg, a nanobody or VHH). In an alternative, BD and BD2 are a VH/VL pair that binds an antigen. The CL of said further polypeptide associates with the CH1 of the other polypeptide (see, eg, FIG. 12I. Optionally in such a polypeptide, dimer or tetramer BD comprises the amino acid of QB-GB (SEQ ID NO: 307), QB-DD, QB-BG or QB-FE (see Table 23 for sequences).

BD and BD2 may be a VH/VL pair of an antigen binding site of an antibody selected from the group consisting of REGN10987, REGN10933 and CB6.

There is also provided:
A protein multimer comprising 4 copies of an antigen binding site of an antibody, wherein the antibody is selected from REGN10987, REGN10933 and CB6.
A protein multimer comprising 4 copies of an antigen binding site of an antibody, wherein the multimer comprises a dimer of an antibody or a fragment thereof (eg, a Fab), wherein the antibody is selected from REGN10987, REGN10933 and CB6.
A protein multimer comprising a dimer of an antibody or a fragment thereof (eg, a Fab), wherein the antibody is selected from REGN10987, REGN10933 and CB6.
A protein multimer comprising 4 (and optionally no more than 4) copies of Nb-112 (SEQ ID NO: 288).
A protein multimer comprising 4 (and optionally no more than 4) copies of an antibody variable domain comprising the amino acid sequence SEQ ID NO: 288.
A protein multimer comprising 8 (and optionally no more than 8) copies of Nb-112 (SEQ ID NO: 288).
A protein multimer comprising 8 (and optionally no more than 8) copies of an antibody variable domain comprising the amino acid sequence SEQ ID NO: 288.
A protein multimer comprising 12 (and optionally no more than 12) copies of Nb-112 (SEQ ID NO: 288).
A protein multimer comprising 12 (and optionally no more than 12) copies of an antibody variable domain comprising the amino acid sequence SEQ ID NO: 288.
A protein multimer comprising 16 (and optionally no more than 16) copies of Nb-112 (SEQ ID NO: 288).
A protein multimer comprising 16 (and optionally no more than 16) copies of an antibody variable domain comprising the amino acid sequence SEQ ID NO: 288.
A protein multimer comprising 20 (and optionally no more than 20) copies of Nb-112 (SEQ ID NO: 288).
A protein multimer comprising 20 (and optionally no more than 20) copies of an antibody variable domain comprising the amino acid sequence SEQ ID NO: 288.
A protein multimer comprising 24 (and optionally no more than 24) copies of Nb-112 (SEQ ID NO: 288).
A protein multimer comprising 24 (and optionally no more than 24) copies of an antibody variable domain comprising the amino acid sequence SEQ ID NO: 288.
A protein multimer comprising 28 (and optionally no more than 28) copies of Nb-112 (SEQ ID NO: 288).
A protein multimer comprising 28 (and optionally no more than 28) copies of an antibody variable domain comprising the amino acid sequence SEQ ID NO: 288.
Instead of an antibody variable domain comprising the amino acid sequence SEQ ID NO: 288, the multimer comprises antibody single variable domain Nb11-59 or an antibody single variable domain comprising SEQ ID NO: 293, or an amino acid sequence that is at least 80, 85, 90, 95, 96, 97, 98 or 99% identical to the amino acid sequence of Nb11-59 or SEQ ID NO: 293. Reference is made to Koenig et al 2021 (Science, 2021 Feb 12;371(6530):eabe6230. doi: 10.1126/science.abe6230. Epub 2021 Jan 12, “Structure-guided multivalent nanobodies block SARS-CoV-2 infection and suppress mutational escape”). Instead of an antibody variable domain comprising the amino acid sequence SEQ ID NO: 288 or Nb11-59, the multimer comprises an antibody single variable domain selected from nanobodies A to W disclosed in Koenig et al, the sequences of which are incorporated in their entirety herein for use in a multimer or polypeptide as described herein. Preferably, the domain is nanobody E, U, V or W.
In an alternative, each copy of the variable domain comprises an amino acid sequence that is at least 80, 85, 90, 95, 96, 97, 98 or 99% identical to SEQ ID NO: 288, wherein the variable domain is capable of binding to a SARS-CoV-2 antigen, eg, spike.
In an alternative, each copy of the variable domain is Nb11-59 or an antibody single variable domain comprising SEQ ID NO: 293. Herein, in any part of the disclosure where NB11-59 is mentioned, in an alternative an antibody single variable domain is used wherein the domain comprises a HCDR3 comprising SEQ ID NO: 294.
Usefully, the invention provides a pharmaceutical composition for inhaled delivery to a patient (eg, a patient suffering from or at risk of SARS-CoV-2 infection), wherein the composition comprises a multimer of the invention. Preferably, the multimer comprises copies of Nb-112 or a variable domain comprising SEQ ID NO: 288. Preferably, the multimer comprises copies of Nb11-59 or an antibody single variable domain comprising SEQ ID NO: 293, or an amino acid sequence that is at least 80, 85, 90, 95, 96, 97, 98 or 99% identical to the amino acid sequence of Nb11-59 or SEQ ID NO: 293. The invention also provides a nebuliser or inhaler device comprising such a composition.
There is also provided a multimer or composition of the invention for inhaled administration to a human or animal patient for treating or preventing a lung condition.
There is also provided a multimer or composition of the invention for inhaled administration to a human or animal patient for treating or preventing a SARS-CoV-2 infection.
There is also provided a multimer or composition of the invention for inhaled administration to a human or animal patient for treating or preventing an inflammatory condition.
There is also provided a multimer or composition of the invention for inhaled administration to a human or animal patient for treating or preventing pneumonia.
There is also provided a multimer or composition of the invention for inhaled administration to a human or animal patient for treating or preventing a cough.
There is also provided a multimer or composition of the invention for inhaled administration to a human or animal patient for treating or preventing loss of smell and/or taste.
There is also provided a multimer or composition of the invention for inhaled administration to a human or animal patient for treating or preventing an elevated temperature (eg, a temperature greater than 38, 39 or 40° C.entrigrade, eg, 37.8° C.entrigrade or greater).
There is also provided a multimer or composition of the invention for inhaled administration to a human or animal patient for treating or preventing COVID or a symptom thereof.
Advantageously, the multimer may comprise mammalian cell glycosylation. The multimer may, for example, comprise 8, 12, 16, 20 or 24 copies of the binding site (eg, VHH). The multimer may contain 4 (but no more than 4) copies of the binding site (eg, VHH). The binding site may be anti-SARS-CoV-2 antigen VH/VL pair comprised by the antibody.

Advantageously, as shown in Example 33, a VH or VHH herein may be a VH3 family VH or VHH. As shown in the example, multimerization of such a variable domain can surprisingly produce a multimer of the invention that can be readily purified by binding to protein A. Thus, preferably, the multimer can be devoid of an affinity tag, such as a His tag.

Herein, any Fc may be a human antibody Fc. Herein, an Fc may be a gamma antibody Fc, mu antibody Fc, delta antibody Fc, epsilon antibody Fc or alpha antibody Fc, preferably a gamma (eg, gamma-1, gamma-2, gamma-3 or gamma-4) antibody Fc (preferably a gamma-1 antibody Fc).

Optionally, the invention provides a protein multimer comprising the configuration of ACE2-TD shown in FIG. 54B. The multimer may comprise further moieties, such as protein domains or peptides than are shown in the figure.

Optionally, the invention provides a protein multimer comprising the configuration of ACE2 monomeric Ig-TD shown in FIG. 54C. The multimer may comprise further moieties, such as protein domains or peptides than are shown in the figure.

Optionally, the invention provides a protein multimer comprising the configuration of ACE2 dimer-TD shown in FIG. 54D. The multimer may comprise further moieties, such as protein domains or peptides than are shown in the figure.

Optionally, the invention provides a protein multimer comprising the configuration of ACE2- Ig-TD shown in FIG. 54E. The multimer may comprise further moieties, such as protein domains or peptides than are shown in the figure.

Optionally, the invention provides a protein multimer comprising the configuration of BD-Heavy Chain Only -TD shown in FIG. 55C. The multimer may comprise further moieties, such as protein domains or peptides than are shown in the figure.

Optionally, the invention provides a protein multimer comprising the configuration of BD-Ig-TD shown in FIG. 55B. The multimer may comprise further moieties, such as protein domains or peptides than are shown in the figure.

Optionally, the invention provides a protein multimer comprising the configuration of BD-Fab-like -TD shown in FIG. 55B. The multimer may comprise further moieties, such as protein domains or peptides than are shown in the figure.

Optionally, the invention provides a protein multimer comprising the configuration of BD-Fab-like monomeric Ig-TD shown in FIG. 55E. The multimer may comprise further moieties, such as protein domains or peptides than are shown in the figure.

Optionally, the invention provides a protein multimer comprising the configuration of Dimeric-TD shown in FIG. 55H. The multimer may comprise further moieties, such as protein domains or peptides than are shown in the figure.

Optionally, the invention provides a protein multimer comprising the configuration of BD-Fab′-like -TD shown in FIG. 55F. The multimer may comprise further moieties, such as protein domains or peptides than are shown in the figure.

Optionally, the invention provides a protein multimer comprising the configuration of BD-monomeric Ig-TD shown in FIG. 55G. The multimer may comprise further moieties, such as protein domains or peptides than are shown in the figure.

Optionally, BD-TD denotes the binding domain directly linked N-terminal to the TD.

Optionally, TD-BD denotes the binding domain directly linked C-terminal to the TD.

Optionally, BD-BD denotes the binding domains directly linked to each other.

Optionally, 2 copies of the polypeptide are associated or joined together so that the N- and C-termini of each polypeptide is not directly joined to the other polypeptide to form a polypeptide dimer (eg, see FIGS. 54D or 55C). For example, the polypeptides of the dimer are disulphide bonded to each other. For example, Fc dimerisation between 2 copies of the polypeptide produce a dimer, wherein the polypeptide comprises an antibody Fc. The invention, in an embodiment, provides a multimer (ie, tetramer) comprising 2 identical copies of the dimer (ie, comprising 4 copies of the polypeptide), wherein the multimer comprises at least 4 copies of BD. For example, each polyeptide has only one BD, wherein the multimer has 4 copies of BD. In another example, each polyeptide has only 2 copies of BD, wherein the multimer has 8 copies of BD. In another example, each polyeptide has only 3 copies of BD, wherein the multimer has 12 copies of BD.

The BD or binding domain herein may be a binding domain disclosed in Table 23, eg, QB-GB, QB-BG or QB-FE (see Table 23 for sequences), or as disclosed in Table 32. The VH/VL pair may be a VH/VL pair of antibody CR3022: CR3022 VH (ie, QMQLVQSGTEVKKPGESLKISCKGSGYGFITYWIGWVRQMPGKGLEWMGIIYPGDSETR YSPSFQGQVTISADKSINTAYLQWSSLKASDTAIYYCAGGSGISTPMDVWGQGTTVTV) paired with CR3022 VL (ie, DIQLTQSPDSLAVSLGERATINCKSSQSVLYSSINKNYLAWYQQKPGQPPKLLIYWASTRE SGVPDRFSGSGSGTDFTLTISSLQAEDVAVYYCQQYYSTPYTFGQGTKVEIK).

In an alternative, instead of BD, the polypeptide comprises or contains a peptide (eg, an insulin peptide or a superantigen peptide or domain) or a receptor (eg, ACE2 ECD). See, eg, FIG. 54C. Exemplary superantigens (eg protein G, A or L) and peptides and domains thereof are disclosed herein.

The invention comprises a multimer (eg, a multimer described elsewhere herein) comprising 4 copies of a binding domain or a peptide. For example, the binding domain may be any binding domain disclosed herein.

The invention comprises a multimer (eg, a multimer described elsewhere herein) comprising 8 copies of a binding domain or a peptide. For example, the binding domain may be any binding domain disclosed herein.

The invention comprises a multimer (eg, a multimer described elsewhere herein) comprising 16 copies of a binding domain or a peptide. For example, the binding domain may be any binding domain disclosed herein.

The invention comprises a multimer (eg, a multimer described elsewhere herein) comprising 20 copies of a binding domain or a peptide. For example, the binding domain may be any binding domain disclosed herein.

The invention comprises a multimer (eg, a multimer described elsewhere herein) comprising 24 copies of a binding domain or a peptide. For example, the binding domain may be any binding domain disclosed herein.

Herein, by “comprising” said number of copies of the binding domain or peptide, the multimer may, for example, comprise no more than said number of the domain or peptide. For example, the multimer may contain exactly said number of copies of the binding domain or peptide.

The invention comprises a multimer (eg, a multimer described elsewhere herein) comprising 4 copies of a first binding domain or a peptide; and 4 copies of a second binding domain or a peptide, wherein the first and second binding domains are different (eg, they bind to different antigens of a virus, eg, a coronavirus or HIV or influenza, or immune checkpoint antigens, or cytokine antigens, or growth factor antigens, or venom (eg, snake venom) antigens). For example, the first binding domain may be any binding domain disclosed herein. For example, the second binding domain may be any binding domain disclosed herein.

The invention comprises a multimer (eg, a multimer described elsewhere herein) comprising 4 or 8 copies of a first binding domain or a peptide; and 4 copies of a second binding domain or a peptide, wherein the first and second binding domains are different (eg, they bind to different antigens of a virus, eg, a coronavirus or HIV or influenza, or immune checkpoint antigens, or cytokine antigens, or growth factor antigens, or venom (eg, snake venom) antigens). For example, the first binding domain may be any binding domain disclosed herein. For example, the second binding domain may be any binding domain disclosed herein.

The invention comprises a multimer (eg, a multimer described elsewhere herein) comprising 4 or 8 copies of a first binding domain or a peptide; and 8 copies of a second binding domain or a peptide, wherein the first and second binding domains are different (eg, they bind to different antigens of a virus, eg, a coronavirus or HIV or influenza, or immune checkpoint antigens, or cytokine antigens, or growth factor antigens, or venom (eg, snake venom) antigens). For example, the first binding domain may be any binding domain disclosed herein. For example, the second binding domain may be any binding domain disclosed herein.

The invention comprises a multimer (eg, a multimer described elsewhere herein) comprising 4 copies of a first binding domain or a peptide; 4 copies of a second binding domain or a peptide; and 4 copies of a third binding domain or a peptide, wherein the first, seond and third binding domains are different from each other (eg, they bind to different antigens of a virus, eg, a coronavirus or HIV or influenza, or immune checkpoint antigens, or cytokine antigens, or growth factor antigens, or venom (eg, snake venom) antigens). For example, the first binding domain may be any binding domain disclosed herein. For example, the second binding domain may be any binding domain disclosed herein. For example, the third binding domain may be any binding domain disclosed herein.

The invention comprises a multimer (eg, a multimer described elsewhere herein) comprising 4 copies of a first binding domain or a peptide; 4 copies of a second binding domain or a peptide; 4 copies of a third binding domain or a peptide; and 4 copies of a fourth binding domain or a peptide, wherein the first, second, third and fourth binding domains are different from each other (eg, they bind to different antigens of a virus, eg, a coronavirus or HIV or influenza, or immune checkpoint antigens, or cytokine antigens, or growth factor antigens, or venom (eg, snake venom) antigens). For example, the first binding domain may be any binding domain disclosed herein. For example, the second binding domain may be any binding domain disclosed herein. For example, the third binding domain may be any binding domain disclosed herein. For example, the fourth binding domain may be any binding domain disclosed herein.

Optionally, the multimer comprises said number of first and second binding domains. Optionally, the multimer comprises said number of first and second peptides.

Optionally, the multimer comprises mammalian cell (eg, human cell) glycosylation.

Optionally, the multimer binds to the antigen with an affinity of less than 200, 150, 100, 90, 80, 70, 60, 50, 40, 35, 30, 25, 20, 15 or 10 pM (preferably less than 40 or 20 pM) in an ELISA assay, such as an ELISA assy disclosed herein. Optionally, the multimer binds to the antigen with an affinity of less than 200, 150, 100, 90, 80, 70, 60, 50, 40, 35, 30, 25, 20, 15 or 10 pM (preferably less than 40 or 20 pM) in an SPR assay, such as an SPR assy disclosed herein. In an embodiment of these options, the multimer comprises or contains 16 copies of a binding domain or peptide.

Optionally, the multimer neutralises the antigen with an IC₅₀ of less than 0.2, 0.1, 0.09, 0.08, 0.07, 0.06, 0.05, 0.04, 0.03, 0.02 or 0.01 nM (preferably from 0.06 to 0.01 nM) in an ELISA assay, such as an ELISA assay disclosed herein. Optionally, the multimer neutralises the antigen with an IC₅₀ of less than 0.2, 0.1, 0.09, 0.08, 0.07, 0.06, 0.05, 0.04, 0.03, 0.02 or 0.01 nM (preferably from 0.06 to 0.01 nM) in an SPR assay, such as an SPR assay disclosed herein. In an embodiment of these options, the multimer comprises or contains 16 copies of a binding domain or peptide.

In an embodiment, the multimer comprises anti-coronavirus (eg, SARS-Cov-2) spike protein binding sites or receptor peptides, wherein the multimer binds to spike trimer or spike RBD. For example, the binding is with an affinity of less than 200, 150, 100, 90, 80, 70, 60, 50, 40, 35, 30, 25, 20, 15 or 10 pM (preferably less than 40 or 20 pM) in an SPR assay, such as an SPR assay disclosed herein; or in an ELISA assay, such as an ELISA assay disclosed herein (eg, an assay as disclosed in Example 28). See, eg, Example 28.

An antigen binding domain or site comprised by a polypeptide or multimer of the invention may be any binding domain or binding site selected from those disclosed herein (eg, any VH, VL, dAb, VHH or scFv) or may be a binding domain or binding site that comprises an amino acid sequence that is at least 70, 80, 85, 90, 95, 96, 97, 98 or 99% identical to the amino acid sequence of said selected domain or site.

An antigen binding domain or site comprised by a polypeptide or multimer of the invention may be any binding domain or binding site selected from those disclosed herein (eg, any VH, VL, dAb, VHH or scFv) or may be a binding domain or binding site that comprises an amino acid sequence that is identical to the amino acid sequence of said selected domain or site except for 10, 9, 8, 7, 6, 5, 4, 3, 2, or 1 amino acid differences (eg, conseravative amino acid changes).

An antigen binding domain or site comprised by a polypeptide or multimer of the invention may be a domain or site that competes with any binding domain or binding site selected from those disclosed herein (eg, any VH, VL, dAb, VHH or scFv) for binding to the antigen. Competition may be determined by a standard competition assay, such as an SPR competition assay or an ELISA assay.

A polypeptide of the invention may have a configuration shown for a polypeptide in any of the figures herein. A multimer (eg, polyeptide dimer or tetramer) of the invention may have a configuration shown for a multimer in any of the figures herein.

In a configuration, the invention provides:
A protein multimer comprising or containing 8 copies of a peptide or an antigen binding site, (optionally wherein the antigen is a virus spike protein of a first virus, optionally wherein the multimer is capable of binding to the first and a second virus, wherein the viruses are different).
The term “comprising” is open language wherein more than 8 copies of the peptide or binding site are possible in embodiments of the multimer. The term “containing” is closed language wherein 8 (but not more or less than 8) copies are present in the multimer. The term “comprising or containing” or “comprises or contains” herein is to be construed accordingly.

In an alternative, the multimer comprises or contains 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 copies of the peptide or binding site. Preferably, the multimer comprises or contains 4, 8, 12, 16, 20, 24, 28, 32 or 36 copies of the peptide or binding site. For example, the binding site is a VH/VL pair comprising VH-ICC paired with VL-ICC or VH-IHG paired with VL-IHG. For example, the binding site comprises QB-GB, QB-DD, QB-BG or QB-FE.

Optionally, the multimer comprises or contains 16 copies of the peptide or binding site.

Optionally, the multimer comprises (i) 4 copies of a polypeptide, wherein each polypeptide copy comprises a tetramerization domain and 2 or more (eg, 2, 3 or 4) copies of the peptide or binding site; or (ii) 4 copies of a dimer of a polypeptide, wherein each polypeptide copy comprises a tetramerization domain and 1 or more (eg, 2) copies of the peptide or binding site.

Optionally, the polypeptide comprises a self-assembly multimerization domain (SAM) (preferably a tetramerization domain (TD)) and one or more copies of an antigen binding site (BD), the polypeptide comprising or consisting of, in N- to C-terminal direction
BD-TD;
TD-BD;
BD-BD-TD;
TD-BD-BD;
BD-TD-BD-BD;
BD-BD-TD-BD; or
BD-BD-TD-BD-BD.

Optionally, the BD is a single variable domain.

Optionally, the BD comprises the amino acid sequence of QB-GB (SEQ ID NO: 307), QB-DD, QB-BG or QB-FE.

Optionally, the polypeptide comprises, in N- to C-terminal direction, BD-CH1-TD, BD-CL-TD, BD-CH1-Fc-TD or BD-Fc-TD, where BD is an antibody V domain (eg, a VH), Fc is an antibody Fc region, and CH1 is an antibody CH1 domain; and optionally each BD-CH1-TD or BD-CH1-Fc-TD polypeptide of the multimer is paired with a respective second polypeptide, wherein the second polypeptide comprises, in N- to C-terminal direction BD2-CL, wherein BD2 is an antibody V domain (eg, a VL or single variable domain), wherein the CH1 pairs with the CL.

In an example, tandam dAbs are provided N-termial to the TD, preferably at the N-terminus of the polypeptide. For example the polypeptide comprises, in N- to C-terminal direction, BD′-optionaly linker-BD-CH1-TD, BD′-optionaly linker-BD -CL-TD, BD′-optionaly linker-BD -CH1-Fc-TD or BD′-optionaly linker-BD -Fc-TD, wherein each of BD and BD′ is an antibody single variable domain (eg, a nanobody), Fc is an antibody Fc region, and CH1 is an antibody CH1 domain. BD′ and BD may be the same or different (eg, comprising different antigen specificities).

In an example, the dimer comprises a first polypeptide comprising, in N- to C-terminal direction, BD-hinge-TD; BD′-optional linker-BD-Hinge-TD; or BD-optional linker-CH1-Hinge-TD. Optionally the first polypeptide is associated with a second polypeptide. In an embodiment, the second polypeptide comprises, in N- to C-terminal direction, BD″-optional Linker 1-BD-optional linker 2-CL (kappa or lambda) (eg, BD″- Linker 1-BD-optional linker 2-CL; BD″-BD-optional linker-CL; or BD″-BD-CL), wherein the first polyeptide comprises a CH1 domain that is paired with the CL, and wherein each of BD and BD′ is an antibody single variable domain (eg, a nanobody).

Optionally, (i) each of BD and BD2 is an antibody single variable domain; or (ii) BD1 is an antibody VH domain and BD2 is an antibody VL domain, wherein the VH and VL form a VH/VL pair comprising an antigen binding site.

Optionally, the polypeptide comprises, in N- to C-terminal direction, BD-CH1-Fc-TD or BD-CH1-Linker-Fc-TD (optionally wherein the Linker is an antibody hinge, wherein the hinge is devoid of a core hinge region).

In configuration, the invention further provides:-
A protein dimer containing 2 copies of the polypeptide.
A protein dimer containing 2 copies of a polypeptide as described herein.
A protein dimer comprising first and second polypeptides, wherein each polypeptide is a polypeptide disclosed herein.
A 4-chain multimer (eg, an antibody) comprising a dimer of the invention, wherein a first polypeptide of the dimer is associated with a second polypeptide of the dimer, wherein a third polypeptide is associated with the first polypeptide and a fourth polypeptide is associated with the second polypeptide. For example, the first and second polypeptides are antibody heavy chains and the third and fourth polypeptides are light chains. For example, the first and third polypetides are associated together and comprise a first antigen binding site that is capable of binding to a first antigen; and the second and fourth polypeptides are associated together and comprise a second antigen binding site that is capable of binding to a second antigen. Optionally, the first and second antigens are different. Optionally, the first and second binding sites are different. For example, each binding site comprises a VH/VL pair. For example, the first and third polypeptides comprise first and second single variable domains, wherein each single variable domain is capable of binding a respective antigen (eg, different antigens) and/or (i) the second and fourth polypeptides comprise third and fourth single variable domains, wherein each single variable domain is capable of binding a respective antigen (eg, different antigens) or (ii) the second and fourth polypeptides are associated together and comprise a VH/VL pair that is capable of binding to an antigen.
Optionally, a multimer herein is multispecific for antigen binding, eg, bispecific, trispecific or tetraspecific.
The polypeptides are associated together, eg, a Fc region of a first polypeptide of the dimer is associated with a Fc of a second polypeptide of the dimer. In an example, each polypeptide comprises a TD.

Optionally, the (or the first) polypeptide comprises, in N- to C-terminal direction, BD-TD.

Optionally, the 2 copies of the polypeptide are disulphide bonded together in the dimer.

The invention also provides:

A protein dimer containing 2 copies of a polypeptide recited herein, wherein the polypeptide comprises BD-CH1-Fc-TD, wherein the Fc regions of the polypeptides associate with each other to form the dimer.

The invention also provides:

A multimer comprising or containing 4 copies of the dimer of the invention.

The invention also provides:

A polypeptide as recited for the multimer or dimer of the invention.

Optionally, the polypeptide is isolated or recombinant.

The multimer, dimer or polypeptide may be comprised by a medical or sterile container, eg, a syringe, vial, IV bag, container connected to a needle or a subcutaneous injection administration device.

Optionally, the antigen is a viral antigen, bacterial antigen, fungal antigen, toxin antigen, venom antigen, immune checkpoint protein antigen, cytokine antigen, growth factor antigen, hormone antigen (eg, chorionic gonadotropin), sugar antigen, lipid antigen or protein antigen.

Optionally, BD and BD2 are different from each other and each comprises a binding site for an antigen of a virus, an antigen of a bacterium, an antigen of a fungus, an antigen of a toxin, an antigen of a venom, an antigen of an immune checkpoint protein, an antigen of a cytokine, an antigen of a growth factor antigen or an antigen of a hormone; optionally wherein both BD and B2 comprises a binding site for a virus.

Optionally, the multimer binds to the antigen with an affinity of less than 200 pM in an ELISA assay; and/or the multimer neutralises the antigen with an IC₅₀ of less than 0.2 nM in an ELISA assay.

Optionally, the multimer is capable of detectably binding to anti-first antigen antibodies (optionally anti-SARS-Cov-2 spike antibodies) in an ELISA assay, wherein detection of the multimer binding is measured by OD₄₅₀ and the assay comprises
(a) Diluting a serum sample of a mammal between 100 and 10⁶-fold;
(b) Contacting the antigen (eg, SARS-Cov-2 spike protein) with the serum sample (which has been diluted in step (a)) whereby anti-first antigen (eg, anti-SARS-Cov-2 spike) antibodies present in the sample bind to the antigen (eg, spike protein), wherein the antigen protein is immobilised on a solid surface;
(c) Contacting the bound antibodies with copies of the multimer of any preceding claim and
(d) Detecting multimer bound to antibody.

Optionally, the dilution is 1000 to 1,000,000, 100,000 or 10000-fold (preferably 10,000 to 100,000-fold).

The invention provides:

A method of detecting the presence of anti-first antigen antibodies (eg, anti-SARS-Cov-2 spike antibodies) in a bodily fluid sample of a human or animal, the method comprising carrying out an ELISA assay, and the assay comprises
(a) Optionally diluting the serum sample from 10 to 10⁶-fold;
(b) contacting the first antigen (eg, SARS-Cov-2 spike protein) with the sample (optionally which has been diluted in step (a)) whereby anti-first antigen (eg, anti-SARS-Cov-2 spike) antibodies present in the sample bind to the first antigen (eg, spike protein) to produce antigen/antibody complexes; and
(c) contacting and binding the first antigen or anti-first antigen (eg, anti-SARS-Cov-2 spike) antibodies with copies of the multimer of any one of claims 1 to 9, 14 and 16 to 19 and
(d) detecting multimer bound to antigen/antibody complexes, the detecting optionally comprising detection of the multimer binding by determining optical density, such as OD₄₅₀;
(e) wherein the steps can be carried out in the order (a) (b) (c) and (d) or (a) (c) (b) and (d), or wherein steps (b) and (c) are carried out simultaneously and between steps (a) and (d).

Optionally, the presence of anti-first antigen antibodies in the sample is detected when the optical density (eg, OD₄₅₀) is greater than 0.1 or 0.5 (optionally, greater than 1, 1.5 or 2) in the assay. Optionally, the dilution is 1000 to 1,000,000, 100,000 or 10000-fold (preferably 10,000 to 100,000-fold).

Optionally, the binding site is
(a) QB-GB, QB-DD, QB-FE or QB-BG;
(b) The spike protein binding site of an antibody selected from 80R, CR3014, CR3006, CR3013 and CR3022;
(c) An anti-SARS-Cov-2 antigen binding site of an antibody selected from REGN10987, REGN10933, CB6, rRBD-15, B38, H4, FYB-207, ABP300, BRII-198, BRII-196, CT-P59, HFB-3013, HFB30132A, MW33, SAB-185, Etesevimab, SCTA01, H014, STI-1499, COVI-GUARD™, TY027, COVI-AMG™, STI-2020, HLX70, ADM03820, an XAV-19 antibody, BGB DXP-593, DXP-604, VIR-7831, GSK4182136, AZD8895, AZD1061, HBM9022, 47D11, Ab8, MAbCo19, AR-701, AR-711, DXP-604, Centi-B9, GIGA-2050, TATX-03, TATX-06, TATX-09, TATX-13, TATX-16, NOVOAB-20, COVI-SHIELD™, STI-4920, ACE-MAB, CMAB020, IDB003 and VIR-7832;
(d) A viral antigen binding site of an antibody selected from the antibodies of Table 21;
(e) A viral antigen binding site disclosed in Table 23 or Table 32;
(f)An ACE2 protein which is capable of binding to the first virus spike protein;
(g) An ACE2 protein disclosed in Table 24;
(h) A TMPRSS2 protein which is capable of binding to the first virus spike protein;
(i) Protein G or a fragment thereof;
(j) Protein A or a fragment thereof;
(k) Protein L or a fragment thereof;
(l) an Ig binding domain disclosed herein (eg, as disclosed in Table 25);
(m) an antibody VH/VL pair, wherein the VH comprises an amino acid sequence selected from a VH sequence disclosed herein and the VL comprises an amino acid sequence selected from a respective VL sequence disclosed herein (optionally wherein the VH is encoded by the DNA sequence of VH-ICC or VH-IHG and the VL is encoded by the DNA sequence of VL-ICC or VL-IHG respectively);
(n) an antibody single variable domain comprising the amino acid sequence of SEQ ID NO: 288 or an amino acid sequence that is at least 80% (eg, at least 80, 85, 90, 95, 96, 97, 98 or 99%) identical to SEQ ID NO: 288; or
(o) antibody single variable domain Nb11-59 or or an antibody single variable domains comprising SEQ ID NO: 203, or an antibody single variable domain comprising amino acid sequence that is at least 80, 85, 90, 95, 96, 97, 98 or 99% identical to the amino acid sequence of Nb11-59 or SEQ ID NO: 293.

Optionally, the multimers are immobilised on a solid surface; or the first antigen is immobilised on a solid surface.

Optionally, determining optical density (eg, OD₄₅₀) comprises labelling complexes comprising spike protein and multimers with horseradish peroxidase (HRP) and detecting the label (eg, at a wavelength of 450 nm).

For the multimer, dimer, polypeptide or method, each multimer may comprise a polypeptide; or variable domain or binding site amino acid disclosed herein.

The invention provides:

A multimer comprising 4 copies of a binding site for an antigen, wherein the multimer comprises a dimer of an antibody or a dimer of an antigen binding fragment (eg, Fab) of an antibody, optionally wherein the multimer is according to any preceding claim. The antibody can be any antibody disclosed herein, eg, an antibody selected from REGN10987, REGN10933, CB6, rRBD-15, B38, H4, FYB-207, ABP300, BRII-198, BRII-196, CT-P59, HFB-3013, HFB30132A, MW33, SAB-185, Etesevimab, SCTA01, H014, STI-1499, COVI-GUARD™, TY027, COVI-AMG™, STI-2020, HLX70, ADM03820, an XAV-19 antibody, BGB DXP-593, DXP-604, VIR-7831, GSK4182136, AZD8895, AZD1061, HBM9022, 47D11, Ab8, MAbCo19, AR-701, AR-711, DXP-604, Centi-B9, GIGA-2050, TATX-03, TATX-06, TATX-09, TATX-13, TATX-16, NOVOAB-20, COVI-SHIELD™, STI-4920, ACE-MAB, CMAB020, IDB003 and VIR-7832.

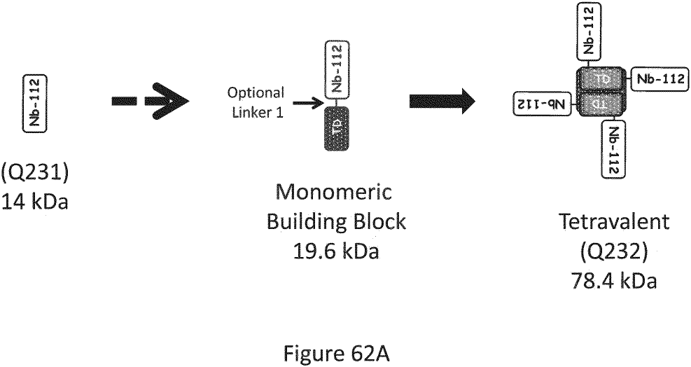

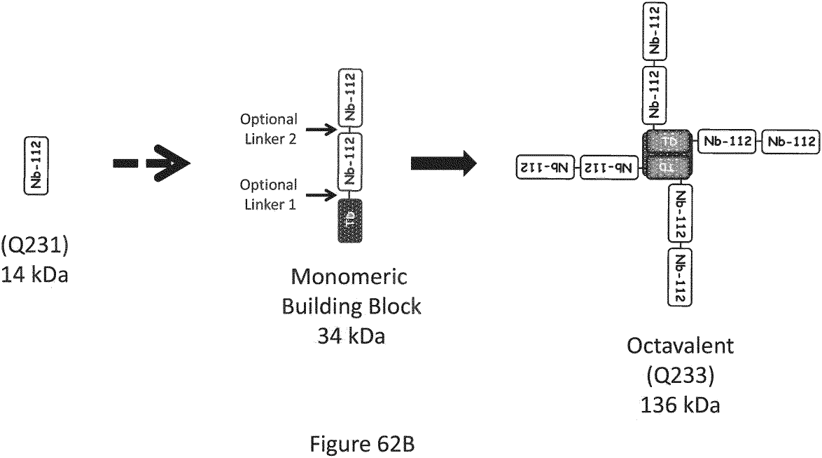

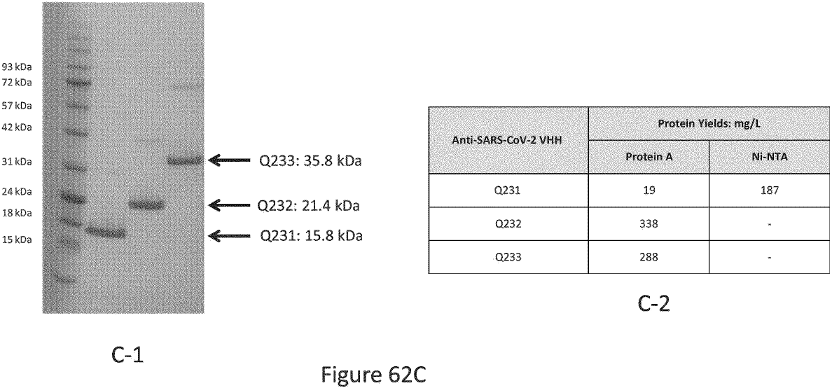

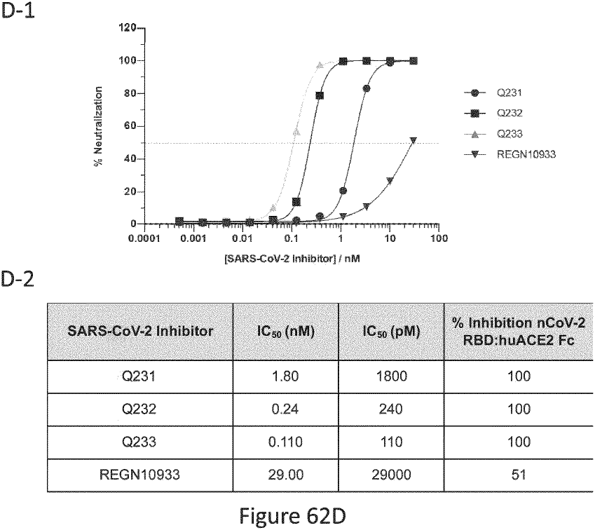

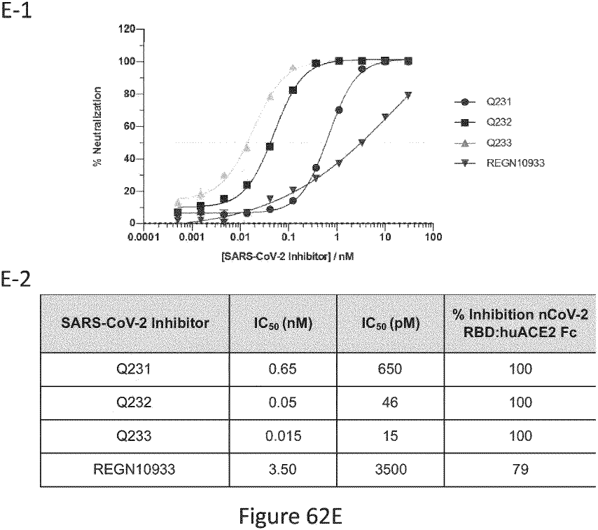

The multimer may be a tetramer having a configuration shown in the right-hand-side schematic of any one of FIGS. 14C, 14-D, 15-I, 15-J and 16-A to 16-C(eg, FIGS. 16A, 16B or 16C, optionally wherein the VH and VL pair is a VH/VL pair of an antigen binding site of an antibody selected from the group consisting of REGN10987, REGN10933 and CB6); or wherein the polypeptdide herein may be a polypeptide having a configuration shown in the middle schematic of any one of FIGS. 14C, 14-D, 15-I, 15-J and 16-A to 16-C; or the dimer may be a dimer of any such polypeptide. The multimer may be a tetramer having a configuration shown in the right-hand-side schematic of FIGS. 62A or 62B.

Optionally,
(a) the multimer is a tetramer having a configuration shown in the right-hand-side schematic of any one of FIGS. 14C, 14-D, 15-I, 15-J and 16-A to 16-C(eg, FIGS. 16A, 16B or 16C, optionally wherein the VH and VL pair is a VH/VL pair of an antigen binding site of an antibody selected from the group consisting of REGN10987, REGN10933 and CB6); or wherein the polypeptdide is a polypeptide having a configuration shown in the middle schematic of any one of FIGS. 14C, 14-D, 15-I, 15-J and 16-A to 16-C; or the dimer is a dimer of any such polypeptide; optionally wherein each VH and each VL is a VH and VL of an antigen binding site of an antibody selected from the group consisting of REGN10987, REGN10933 and CB6;
(b) the multimer is a tetramer of 4 copies of a polypeptide, wherein each polypeptide comprises an amino acid sequence selected from SEQ ID NOs: 232, 233, 234, 235, 236, 237, 238 (optionally wherein the polypeptide is paired with a polypeptide comprising SEQ ID NO: 240), 239 (optionally wherein the polypeptide is paired with a polypeptide comprising SEQ ID NO: 287), 241, 242, 243, 244, 245 (optionally wherein the polypeptide is paired with a polypeptide comprising SEQ ID NO: 240 or 286), 246 (optionally wherein the polypeptide is paired with a polypeptide comprising SEQ ID NO: 240), 247 (optionally wherein the polypeptide is paired with a polypeptide comprising SEQ ID NO: 287), 248 (optionally wherein the polypeptide is paired with a polypeptide comprising SEQ ID NO: 240), 249 (optionally wherein the polypeptide is paired with a polypeptide comprising SEQ ID NO: 287), 250 (optionally wherein the polypeptide is paired with a polypeptide comprising SEQ ID NO: 253), 251 (optionally wherein the polypeptide is paired with a polypeptide comprising SEQ ID NO: 253), 252 (optionally wherein the polypeptide is paired with a polypeptide comprising SEQ ID NO: 253), 254 (optionally wherein the polypeptide is paired with a polypeptide comprising SEQ ID NO: 257), 255 (optionally wherein the polypeptide is paired with a polypeptide comprising SEQ ID NO: 257), 256 (optionally wherein the polypeptide is paired with a polypeptide comprising SEQ ID NO: 257), 258 (optionally wherein the polypeptide is paired with a polypeptide comprising SEQ ID NO: 261), 259 (optionally wherein the polypeptide is paired with a polypeptide comprising SEQ ID NO: 261) and 260 (optionally wherein the polypeptide is paired with a polypeptide comprising SEQ ID NO: 261);
(c) the multimer comprises 4 copies of an antigen binding site, wherein the binding site comprises a VH/VL pair, wherein the VH and VL are respectively encoded by the nucleotide sequences of SEQ ID NOs: 268 and 269, 270 and 271, 272 and 273, 274 and 275, 276 and 277, 278 and 279, 280 and 281, 282 and 283, or 284 and 285; the polypeptide comprises a copy of such a binding site; or the dimer comprises 2 copies of such a binding site; or
(d) wherein the multimer comprises 4 copies of an antigen binding site, wherein the binding site comprises a VH/VL pair, wherein the VH and VL respectively comprise the amino acid sequences of SEQ ID NOs: 262 and 263, 264 and 265, or 266 and 267; the polypeptide comprises a copy of such a binding site; or the dimer comprises 2 copies of such a binding site.

The invention provides:

A pharmaceutical composition or assay reagent comprising a plurality of multimers of the invention, optionally wherein the reagent comprises said multimers immobilised on a solid support.

A multimer of the invention (or a combination of at least 2 or 3 multimers of the invention claim) for administration to a human or animal subject for medical use.

There is provided a composition comprising a multimer of the invention, eg, for medical use or for use in vitro.

A VH herein may be a VH encoded by a VH DNA sequence shown in Table 21(b) and a VL herein may be a VL encoded by the cognate VL DNA sequence shown in Table 21(b), wherein the VH and VL form an antigen binding VH/VL pair (eg, that is capable of binding to a SARS-CoV-2 antigen, such as spike antigen). Alternatively, the VH sequence is at least 70, 75, 80, 85, 90, 95, 96, 97, 98 or 99% identical to said VH DNA sequence; and/or the VL sequence is at least 70, 75, 80, 85, 90, 95, 96, 97, 98 or 99% identical to said VL DNA sequence. In an example, the VH DNA sequence is VH-ICC (see Table 21(b)) or a VH sequence that is at least 70, 75, 80, 85, 90, 95, 96, 97, 98 or 99% identical to said VH-ICC DNA sequence; and the VL DNA sequence is VL-ICC (see Table 21(b)) or a VL sequence that is at least 70, 75, 80, 85, 90, 95, 96, 97, 98 or 99% identical to said VL-ICC DNA sequence. In an example, the VH DNA sequence is VH-IHG (see Table 21(b)) or a VH sequence that is at least 70, 75, 80, 85, 90, 95, 96, 97, 98 or 99% identical to said VH-IHG DNA sequence; and the VL DNA sequence is VL-IHG (see Table 21(b)) or a VL sequence that is at least 70, 75, 80, 85, 90, 95, 96, 97, 98 or 99% identical to said VL-IHG DNA sequence.

A VH herein may comprise the amino acid sequence of SEQ ID NO: 288 or an amino acid sequence that is at least 80, 85, 90, 95, 96, 97, 98 or 99% identical to SEQ ID NO: 288. Preferably, such a VH is unpaired with a second variable domain (eg, a VL), since the VH in this instance is a single variable domain, it is able to bind to a SARS-Cov-2 antigen (eg, spike) without requirement for pairing.

A VH herein may comprise antibody single variable domain Nb11-59 (Novamab Biopharmaceuticals Co. Ltd) or an antibody single variable domain of ALX-0171 (Ablynx). A VH herein may comprise antibody single variable domain comprising SEQ ID NO: 203, or an antibody single variable domain comprising amino acid sequence that is at least 80, 85, 90, 95, 96, 97, 98 or 99% identical to the amino acid sequence of Nb11-59 or SEQ ID NO: 293.

Optionally, the combination comprises first and second multimers of the invention, wherein the first multimer comprises a binding site comprising a first VH/VL pair and the second multimer comprises a second VH/VL pair which is different from the first VH/VL pair. In an example, the VH of the first VH/VL pair is encoded by the DNA sequence of VH-ICC and the VL is encoded by the DNA sequence of VL-ICC. In an example, the VH of the first VH/VL pair is encoded by the DNA sequence of VH-IHG and the VL is encoded by the DNA sequence of VL-IHG.

In an example, the VH of the first VH/VL pair is encoded by a DNA sequence that is at least 70, 75, 80, 85, 90, 95, 96, 97, 98 or 99% identical (or 100% identical) to the DNA sequence of VH-ICC and the VL is encoded by a DNA sequence that is at least 70, 75, 80, 85, 90, 95, 96, 97, 98 or 99% identical (or 100% identical) to the DNA sequence of VL-ICC. In an example, the VH of the first VH/VL pair is encoded by the DNA sequence that is at least 70, 75, 80, 85, 90, 95, 96, 97, 98 or 99% identical (or 100% identical) to the DNA sequence of VH-IHG and the VL is encoded by the DNA sequence that is at least 70, 75, 80, 85, 90, 95, 96, 97, 98 or 99% identical (or 100% identical) to the DNA sequence of VL-IHG.

Optionally, the multimer comprises first and second antigen binding sites which are different from each other. For example, the first binding site comprises a first VH/VL pair and the second binding site comprises a second VH/VL pair. In an example, the VH of the first VH/VL pair is encoded by a DNA sequence that is at least 70, 75, 80, 85, 90, 95, 96, 97, 98 or 99% identical (or 100% identical) to a first VH DNA sequence disclosed in Table 21(b) and the VL is encoded by a DNA sequence that is at least 70, 75, 80, 85, 90, 95, 96, 97, 98 or 99% identical (or 100% identical) to the cognate VL DNA sequence disclosed in Table 21(b); and the VH of the second VH/VL pair is encoded by a DNA sequence that is at least 70, 75, 80, 85, 90, 95, 96, 97, 98 or 99% identical (or 100% identical) to a second VH DNA sequence disclosed in Table 21(b) and the VL is encoded by a DNA sequence that is at least 70, 75, 80, 85, 90, 95, 96, 97, 98 or 99% identical (or 100% identical) to the cognate VL DNA sequence disclosed in Table 21(b), wherein the first and second VH DNA sequences are different. For example, the first VH DNA sequence is the sequence of VH-IHI (see Table 21(b)), the cognate VH DNA sequence is VL-IHI; and the second VH DNA sequence is the sequence of VH-IHG, VH-ICC, VH-ICD, VH-IGG, VH-IFD, VH-IED, VH-IHD or VH-IHF. For example, the first VH DNA sequence is the sequence of VH-IHG (see Table 21(b)), the cognate VH DNA sequence is VL-IHG; and the second VH DNA sequence is the sequence of VH-IHI, VH-ICC, VH-ICD, VH-IGG, VH-IFD, VH-IED, VH-IHD or VH-IHF. For example, the first VH DNA sequence is the sequence of VH-ICC (see Table 21(b)), the cognate VH DNA sequence is VL-ICC; and the second VH DNA sequence is the sequence of VH-IHG, VH-IHQ, VH-ICD, VH-IGG, VH-IFD, VH-IED, VH-IHD or VH-IHF. For example, the first VH DNA sequence is the sequence of VH-ICD (see Table 21(b)), the cognate VH DNA sequence is VL-ICD; and the second VH DNA sequence is the sequence of VH-IHG, VH-IHI, VH-ICC, VH-IGG, VH-IFD, VH-IED, VH-IHD or VH-IHF. For example, the first VH DNA sequence is the sequence of VH-IGG (see Table 21(b)), the cognate VH DNA sequence is VL-IGG; and the second VH DNA sequence is the sequence of VH-ICC, VH-ICD, VH-IHI, VH-IFD, VH-IED, VH-IHD or VH-IHF. For example, the first VH DNA sequence is the sequence of VH-IFD (see Table 21(b)), the cognate VH DNA sequence is VL-IFD; and the second VH DNA sequence is the sequence of VH-IHG, VH-IHI, VH-ICC, VH-ICD, VH-GG, VH-IED, VH-IHD or VH-IHF. For example, the first VH DNA sequence is the sequence of VH-IED (see Table 21(b)), the cognate VH DNA sequence is VL-IED; and the second VH DNA sequence is the sequence of VH-IHG, VH-IHI, VH-ICC, VH-ICD, VH-IGG, VH-IFD, VH-IHD or VH-IHF. For example, the first VH DNA sequence is the sequence of VH-IHD (see Table 21(b)), the cognate VH DNA sequence is VL-IHD; and the second VH DNA sequence is the sequence of VH-IHG, VH-IHI, VH-ICC, VH-ICD, VH-GG, VH-IFD, VH-IED, or VH-IHF. For example, the first VH DNA sequence is the sequence of VH-IHF (see Table 21(b)), the cognate VH DNA sequence is VL-IHF; and the second VH DNA sequence is the sequence of VH-IHG, VH-IHI, VH-ICC, VH-ICD, VH-GG, VH-IFD, VH-IED or VH-IHD. Preferably, the second VH DNA sequence is VH-ICC or VH-IHG.

Optionally, the multimer comprises first and second antigen binding sites which are different from each other. For example, the first binding site comprises a first VH/VL pair and the second binding site comprises a second VH/VL pair. In an example, the VH of the first VH/VL pair is encoded by a DNA sequence that is at least 70, 75, 80, 85, 90, 95, 96, 97, 98 or 99% identical (or 100% identical) to the DNA sequence of VH-ICC and the VL is encoded by a DNA sequence that is at least 70, 75, 80, 85, 90, 95, 96, 97, 98 or 99% identical (or 100% identical) to the DNA sequence of VL-ICC; and the VH of the second VH/VL pair is encoded by a DNA sequence that is at least 70, 75, 80, 85, 90, 95, 96, 97, 98 or 99% identical (or 100% identical) to the DNA sequence of VH-IHG and the VL is encoded by a DNA sequence that is at least 70, 75, 80, 85, 90, 95, 96, 97, 98 or 99% identical (or 100% identical) to the DNA sequence of VL-IHG. For example, the multimer comprises (i) at least 4, 8, 12, 16, 20, 24 or 28 (optionally no more than 4, 8, 12, 16, 20, 24 or 28 respectively) copies of a further antibody variable domain that is different from the domain of (i). For example, each domain is capable of specifically binding to a SARS-CoV-2 antigen.

In an embodiment, the multimer comprises a tetramer of a polyeptide dimer, wherein the dimer comprises a first polypeptide associated with a second polypeptide, wherein the first polypeptide comprises at least one copy of a first peptide or a first antigen binding site and a teramerisation domain (TD), the second polypeptide comprises at least one copy of a second peptide or a second antigen binding site and a teramerisation domain (TD).

Preferably, the TDs of the first and second polypeptides are identical. Optionally, the TDs are p53 TDs, such as human p53 TDs. In one embodiment, each of the first and second polypeptides comprises a said peptide and the first and second peptides are different. In one embodiment, each of the first and second polypeptides comprises a said peptide and the first and second peptides are the same. In one embodiment, each of the first and second polypeptides comprises a said binding site and the first and second binding sites are different.

In one embodiment, each of the first and second polypeptides comprises a said binding site and the first and second binding sites are the same. In an example, in each dimer the first and second polypeptides are disulphide bonded together.

In an example, in each dimer the first polypeptide comprises an antibody CH3 domain (eg, a CH2-CH3) and the second polypeptide comprises an antibody CH3 domain (eg, a CH2-CH3), wherein the CH3 domains associate together to form said dimer. For example, each of the first and second polypeptides comprises, in N- to C-terminal direction, a peptide or antigen binding site -TD -optional CH2 domain -CH3 domain, wherein the CH3 domains are associated together. For example, each of the first and second polypeptides comprises, in N- to C-terminal direction, a peptide or antigen binding site - optional CH2 domain - CH3 domain - TD, wherein the CH3 domains are associated together.

“Knobs into holes” technology for making bispecific antibodies was described in [¹] and in US5,731,168, both incorporated herein by reference. The principle is to engineer paired CH3 domains of heterodimeric heavy chains so that one CH3 domain contains a “knob” and the other CH3 domains contains a “hole” at a sterically opposite position. Knobs are created by replacing small amino acid side chain at the interface between the CH3 domains, while holes are created by replacing large side chains with smaller ones. The knob is designed to insert into the hole, to favour heterodimerisation of the different CH3 domains while destabilising homodimer formation. In in a mixture of antibody heavy and light chains that assemble to form a bispecific antibody, the proportion of IgG molecules having paired heterodimeric heavy chains is thus increased, raising yield and recovery of the active molecule

Mutations Y349C and/or T366W may be included to form “knobs” in an IgG CH3 domain. Mutations E356C, T366S, L368A and/or Y407V may be included to form “holes” in an IgG CH3 domain. Knobs and holes may be introduced into any human IgG CH3 domain, e.g., an IgG1, IgG2, IgG3 or IgG4 CH3 domain. A preferred example is IgG4. The IgG4 may include further modifications such as the “P” and/or “E” mutations. A “P” substitution at position 228 in the hinge (S228P) stabilises the hinge region of the heavy chain. An “E” substitution in the CH2 region at position 235 (L235S) abolishes binding to FcyR. A bispecific antibody of the present invention may contain an IgG4 PE human heavy chain constant region, optionally comprising two such paired constant regions, optionally wherein one has “knobs” mutations and one has “holes” mutations.

While knobs-into-holes technology involves engineering amino acid side chains to create complementary molecular shapes at the interface of the paired CH3 domains in the bispecific heterodimer, another way to promote heterodimer formation and hinder homodimer formation is to engineer the amino acid side chains to have opposite charges. Association of CH3 domains in the heavy chain heterodimers is favoured by the pairing of oppositely charged residues, while paired positive charges or paired negative charges would make homodimer formation less energetically favourable. WO2006/106905 described a method for producing a heteromultimer composed of more than one type of polypeptide (such a heterodimer of two different antibody heavy chains) comprising a substitution in an amino acid residue forming an interface between said polypeptides such that heteromultimer association will be regulated, the method comprising:
(a) modifying a nucleic acid encoding an amino acid residue forming the interface between polypeptides from the original nucleic acid, such that the association between polypeptides forming one or more multimers will be inhibited in a heteromultimer that may form two or more types of multimers;
(b) culturing host cells such that a nucleic acid sequence modified by step (a) is expressed; and
(c) recovering said heteromultimer from the host cell culture,
wherein the modification of step (a) is modifying the original nucleic acid so that one or more amino acid residues are substituted at the interface such that two or more amino acid residues, including the mutated residue(s), forming the interface will carry the same type of positive or negative charge.

An example of this is to suppress association between heavy chains by introducing electrostatic repulsion at the interface of the heavy chain homodimers, for example by modifying amino acid residues that contact each other at the interface of the CH3 domains, including:
(a) positions 356 and 439
(b) positions 357 and 370
(c) positions 399 and 409,
the residue numbering being according to the EU numbering system.

By modifying one or more of these pairs of residues to have like charges (both positive or both negative) in the CH3 domain of a first heavy chain, the pairing of heavy chain homodimers is inhibited by electrostatic repulsion. By engineering the same pair or pairs of residues in the CH3 domain of a second (different) heavy chain to have an opposite charge compared with the corresponding residues in the first heavy chain, the heterodimeric pairing of the first and second heavy chains is promoted by electrostatic attraction.

In one example, amino acids at the heavy chain constant region CH3 interface of the dimer of the invention are modified to introduce charge pairs, the mutations being listed in Table 1 of WO2006/106905. It was reported that modifying the amino acids at heavy chain positions 356, 357, 370, 399, 409 and 439 to introduce charge-induced molecular repulsion at the CH3 interface had the effect of increasing efficiency of formation of the intended bispecific antibody. WO2006/106905 also exemplified bispecific IgG antibodies in which the CH3 domains of IgG4 were engineered with knobs-into-holes mutations.

Further examples of charge pairs are disclosed in WO2013/157954, which described a method for producing a heterodimeric CH3 domain-comprising molecule from a single cell, the molecule comprising two CH3 domains capable of forming an interface. The method comprised providing in the cell
(a) (a) a first nucleic acid molecule encoding a first CH3 domain-comprising polypeptide chain, this chain comprising a K residue at position 366 according to the EU numbering system and
(b) (b) a second nucleic acid molecule encoding a second CH3 domain-comprising polypeptide chain, this chain comprising a D residue at position 351 according to the EU numbering system,
the method further comprising the step of culturing the host cell, allowing expression of the two nucleic acid molecules and harvesting the heterodimeric CH3 domain-comprising molecule from the culture.

Further methods of engineering electrostatic interactions in polypeptide chains to promote heterodimer formation over homodimer formation were described in WO2011/143545.

Another example of engineering at the CH3-CH3 interface that can be used in the dimer of the invention is strand-exchange engineered domain (SEED) CH3 heterodimers. The CH3 domains are composed of alternating segments of human IgA and IgG CH3 sequences, which form pairs of complementary SEED heterodimers referred to as “SEED-bodies” [²; WO2007/110205].

Bispecifics have also been produced with heterodimerised heavy chains that are differentially modified in the CH3 domain to alter their affinity for binding to a purification reagent such as Protein A. WO2010/151792 described a heterodimeric bispecific antigen-binding protein comprising
(a) a first polypeptide comprising, from N-terminal to C-terminal, a first epitope-binding region that selectively binds a first epitope, an immunoglobulin constant region that comprises a first CH3 region of a human IgG selected from IgG1, IgG2, and IgG4; and
(b) a second polypeptide comprising, from N-terminal to C-terminal, a second epitope-binding region that selectively binds a second epitope, an immunoglobulin constant region that comprises a second CH3 region of a human IgG selected from IgG1, IgG2, and IgG4, wherein the second CH3 region comprises a modification that reduces or eliminates binding of the second CH3 domain to Protein A.

Thus, in the dimer of the present invention, the CH3 of one (but not the other) of the first and second polypeptides comprises a modification that reduces or eliminates binding of the respective CH3 domain to Protein A.

Dimers and antibodies of the present invention may employ any of these techniques and molecular formats as desired.

Optionally, each of the first and second polypeptides comprises an antigen binding site, wherein each binding site is an antibody single variable domain (eg, a VHH or nanobody).

Optionally, the dimer comprises a third polypeptide and a fourth polypeptide, wherein the third polypeptide is associated with the first polypeptide, and the fourth polypeptide is associated with the second polypeptide, wherein each polyeptide comprises an antibody variable domain, wherein (i) the variable domain of the first polypeptide is paired with the variable domain of the third polypepeptide to form a first VH/VL binding site for binding a first antigen; (ii) and the variable domain of the second polypeptide is paired with the variable domain of the fourth polypepeptide to form a second VH/VL binding site for binding a second antigen. Preferably, the first antigen is different from the second antigen. In an alternative, the first and second antigens are the same. In an example, the variable domain of the first polypeptide is a VH and the variable domain of the third polypeptide is a VL. In an example, the variable domain of the first polypeptide is a VL and the variable domain of the third polypeptide is a VH. In an example, the variable domain of the second polypeptide is a VH and the variable domain of the fourth polypeptide is a VL. In an example, the variable domain of the second polypeptide is a VL and the variable domain of the fourthe polypeptide is a VH.

For example, the first polypeptide comprises a CH1 domain that associates with a CL domain that is comprised by the third polypeptide and/or the second polypeptide comprises a CH1 domain that associates with a CL domain that is comprised by the fourth polypeptide.

For example, (i) the first polypeptide comprises, in N- to C-terminal direction, a peptide or a variable domain of a first VH/VL antigen binding site - CH1 domain - optional hinge region -TD - [a Fc region comprising a CH2 domain and aCH3 domain]; and (ii) the second polypeptide comprises, in N- to C-terminal direction, a peptide or a variable domain of a second VH/VL antigen binding site - CH1 domain - optional hinge region -TD - [a Fc region comprising a CH2 domain and a CH3 domain], wherein the CH3 domains of the first and second polypeptides are associated together. In this example, (i) the third polypeptide comprises, in N- to C-terminal direction, a peptide or a variable region of the first antigen binding site - CL; (ii) the fourth polypeptide comprises, in N- to C-terminal direction, a peptide or a variable region of the second antigen binding site - CL, wherein (iii) said variable domains or the first and third polypeptides form the first VH/VL binding site (eg, wherein the variable domain of the first polypeptide is a VH and the variable domain of the third polypeptide is a cognate VL), (iv) said variable domains of the second and fourth polypeptides form the second VH/VL binding site (eg, wherein the variable domain of the second polypeptide is a VH and the variable domain of the fourth polyeptide is a cognate VL), (v) the CH1 of the first polypeptide is associated with the CL of the third polypeptide, (vi) the CH1 of the second polypeptide is associated with the CL of the fourth polypeptide, and (vii) the Fc of the first polyeptide is associated with the Fc of the seond polypeptide (eg, the CH3 domains are associated together).

Optionally, the Fc regions (or CH3 domains) of the first and second polypeptides are associated together using knob-in-hole technology or charge pairing.

The multimer of the invention may be a multimer for administration to a human or animal subject for treatment or prevention of a disease or condition (eg, an infection by the first and/or second virus, or a symptom of such an infection (eg, an unwanted inflammatory response)) in the subject.

The invention provides:

A method for the treatment or prevention of a disease or condition (eg, an infection by the first and/or second virus, or a symptom of such an infection (eg, an unwanted inflammatory response)) in a human or animal subject, the method comprising administering to the subject a plurality of multimers of the invention.

An assay kit comprising an assay reagent as mentioned above and an amount of the first antigen (eg, viral spike protein), optionally wherein the reagent and protein are comprised by different containers.

A method for detecting the presence of an antigen in a sample, the method comprising combining the sample with a multimer of the invention, allowing antigen in the sample to bind multimers to form antigen/multimer complexes and detecting antigen/multimer complexes.

A method of expanding a utility of an antigen (eg, a protein) binding site, the method comprising producing a multimer of the invention, wherein the multimer comprises a plurality of copies (eg, at least 8 or 16 copies) of the binding site.

Optionally for the multimer, dimer, polypeptide, method, kit or composition, the multimer comprises a tetramer of a polyeptide dimer, wherein the dimer comprises a first polypeptide associated with a second polypeptide, wherein the first polypeptide comprises at least one copy of a first peptide or a first antigen binding site and a teramerisation domain (TD), the second polypeptide comprises at least one copy of a second peptide or a second antigen binding site and a teramerisation domain (TD).

Optionally, each of the first and second polypeptides comprises a said binding site and the first and second binding sites are different.

Optionally, in each dimer the first polypeptide comprises an antibody CH3 domain (eg, a CH2-CH3) and the second polypeptide comprises an antibody CH3 domain (eg, a CH2-CH3), wherein the CH3 domains associate together to form said dimer.

Optionally, each of the first and second polypeptides comprises, in N- to C-terminal direction, (i) a peptide or antigen binding site -TD - optional CH2 domain -CH3 domain, wherein the CH3 domains of the first and second polypeptides are associated together; or (ii) a peptide or antigen binding site - optional CH2 domain - CH3 domain - TD, wherein the CH3 domains of the first and second polypeptide are associated together.

Optionally, each of the first and second polypeptides comprises an antigen binding site, wherein each binding site is an antibody single variable domain (eg, a VHH or nanobody).

Optionally, the dimer is associated with a third polypeptide and a fourth polypeptide, wherein the third polypeptide is associated with the first polypeptide, and the fourth polypeptide is associated with the second polypeptide, wherein each polyeptide comprises an antibody variable domain, wherein (i) the variable domain of the first polypeptide is paired with the variable domain of the third polypepeptide to form a first VH/VL binding site for binding a first antigen; and (ii) the variable domain of the second polypeptide is paired with the variable domain of the fourth polypepeptide to form a second VH/VL binding site for binding a second antigen.

Optionally, the first and second antigens are different.

Optionally, the variable domain of the first polypeptide is a VH and the variable domain of the third polypeptide is a VL and/or the variable domain of the second polypeptide is a VH and the variable domain of the fourth polypeptide is a VL.

Optionally, (A) (i) the first polypeptide comprises a CH1 domain that associates with a CL domain that is comprised by the third polypeptide and (ii) the second polypeptide comprises a CH1 domain that associates with a CL domain that is comprised by the fourth polypeptide; or (B) (i) the first polypeptide comprises a CH1 domain that associates with a CL domain that is comprised by the third polypeptide and (ii) the second polypeptide comprises a CL domain that associates with a CH1 domain that is comprised by the fourth polypeptide.

In an embodiment of option (A), the third and fourth polypeptides are identical. Thus, a common chain or polypeptide is used. Thus, the common polypeptide associates with each of the first and second polypeptides. This may simplify production by requiring only 3, instead of 4 different polypeptides to be expressed together.

Option (B) is useful to reduce chances of undesirable light chain pairing, ie, the fourth polypeptide pairing with the first polypeptide and/or the third polypeptide pairing with the second polypeptide. Thus, having the CH1 in the first polypeptide and the CL in the third polypeptide, this avoids the risk of the third polypeptide pairing with the second polypeptide, since these two polypeptides comprise CL domains that do not pair with each other. Similarly, the CH domains of the first and fourth polypeptides do not pair with each other. This is advantageous for favouring production of multimers of the invention where there is a first/third polypeptide pair and a second/fourth polypeptide pair comprised by each dimer of the multimer. For example (i) the first/third polypeptide pair comprises the following configuration wherin the first polypeptide comprises in N- to C-terminal direction [VH-CL-Hinge-CH2-CH3-TD] paired with the third polypeptide wherein the third polypeptide comprises in N- to C-terminal direction [VL-CH1]; and (ii) the second/fourth polypeptide pair comprises the following configuration wherein the second polyeptide comprises in N- to C-terminal direction [VH-CH1-Hinge-CH2-CH3-TD] paired with the fourth polypeptide wherein the fourth polypeptide comprises in N- to C-terminal direction [VL-CL].

Optionally:
(A)
   (i) the first polypeptide comprises, in N- to C-terminal direction, a variable domain of a first VH/VL antigen binding site - CH1 domain - optional hinge region - [a Fc region comprising a CH2 domain and a CH3 domain];
   (ii) the second polypeptide comprises, in N- to C-terminal direction, a variable domain of a second VH/VL antigen binding site - CH1 domain - optional hinge region - [a Fc region comprising a CH2 domain and a CH3 domain];
   (iii) the third polypeptide comprises, in N- to C-terminal direction, a variable region of the first antigen binding site - CL;
   (iv) the fourth polypeptide comprises, in N- to C-terminal direction, a variable region of the second antigen binding site - CL;
   (v) said variable domains or the first and third polypeptides form the first VH/VL binding site (eg, wherein the variable domain of the first polypeptide is a VH and the variable domain of the third polypeptide is a cognate VL);
   (vi) said variable domains of the second and fourth polypeptides form the second VH/VL binding site (eg, wherein the variable domain of the second polypeptide is a VH and the variable domain of the fourth polyeptide is a cognate VL);
   (vii) the CH1 of the first polypeptide is associated with the CL of the third polypeptide; (viii) the CH1 of the second polypeptide is associated with the CL of the fourth polypeptide, and (ix) the Fc of the first polyeptide is associated with the Fc of the second polypeptide (eg, the CH3 domains are associated together); or
(B)
   (i) the first polypeptide comprises, in N- to C-terminal direction, a first antibody single variable domain - CH1 domain - optional hinge region - [a Fc region comprising a CH2 domain and a CH3 domain];
   (ii) the second polypeptide comprises, in N- to C-terminal direction, a second antibody single variable domain - CH1 domain - optional hinge region - [a Fc region comprising a CH2 domain and a CH3 domain]; and
   (ix) the Fc of the first polyeptide is associated with the Fc of the second polypeptide (eg, the CH3 domains are associated together).

Optionally, the Fc regions of the first and second polypeptides are associated by knob-in-hole or charge pairing technology.

Optionally:
(i) each of the first and second polypeptides comprises a respective TD between the CH1 and the Fc thereof;
(ii) each of the first and second polypeptides comprises a hinge reion and each of said polypeptides comprises a respective TD between the hinge region and the Fc thereof; or
(iii) each of the first and second polypeptides comprises in N- to C-terminal direction the Fc thereof and a respective TD.

Optionally, (i) the first/third polypeptide pair comprises a configuration wherein the first polypeptide comprises in N- to C-terminal direction [VH-CL-Hinge-CH2-CH3-TD] paired with the third polypeptide comprising in N- to C-terminal direction [VL-CH1]; and (ii) the second/fourth polypeptide pair comprises a wherein the second polyeptide comprises in N- to C-terminal direction [VH-CH1-Hinge-CH2-CH3-TD] paired with the fourth polypeptide wherein the fourth polypeptide comprises in N- to C-terminal direction [VL-CL].

Optionally, the Fc regions of the first and second polypeptides are associated using knob-in-hole technology, wherein (i) the Fc of the first polypeptide comprises a CH3 domain having a knob that associates with a hole of a CH3 domain of the Fc of the second polypeptide; or (ii) the Fc of the first polypeptide comprises a CH3 domain having a hole that associates with a knob of a CH3 domain of the Fc of the second polypeptide.

Optionally, the Fc regions of the first and second polypeptides are associated using charge pairing technology, wherein (i) the Fc of the first polypeptide comprises a first amino acid positive charge that associates with a second amino acid negative charge of the Fc of the second polypeptide; or (ii) the Fc of the first polypeptide comprises a first amino acid negative charge that associates with a second amino acid positive charge of the Fc of the second polypeptide.

In an alternative, the first, but not the second, polypeptide comprises a TD. In another alternative each of the first and second polypeptides are devoid of a TD.

In an alternative, the dimer of the invention may be devoid of a TD, wherein the Fc regions of the first and second polypeptides are associated together in the dimer. For such an embodiment where the dimer is devoid of a TD, all other features of the dimer disclosed herein (in respect of dimers comprsing a TD) are otherwise applicable mutatis mutandis and combinable with the alternative emobidment that is devoid of a TD. For example, the Fc regions may be associated using any technology described herein, such as using knob-in-hole or charge pairing technology. In an embodiment, the first polyeptide comprises a first antigen binding site (eg, a single variable domain or a variable domain that is paired with a variable domain of the third polypeptide (when present) to form a first VH/VL binding site) and/or the second polyeptide comprises a second antigen binding site (eg, a single variable domain or a variable domain that is paired with a variable domain of the fourth polypeptide (when present) to form a second VH/VL binding site).

Optionally, the dimer is an antibody and the first polypeptide is a first heavy chain, the second polypeptide is a second heavy chain, the third polypeptide is a first light chain and the fourth polypeptide is a second light chain. For example, the first and second heavy chains are identical. In an example, they are different (eg, they comprise different peptides or they comprise different antigen binding sites or V domains). For example, the first and second light chains are identical. In an example, they are different (eg, they comprise different different peptides or they comprise different antigen binding sites or V domains). In an embodiment the dimer is a bispecific antibody wherein the first and second heavy chains comprise different binding sites capable of binding a respective antigen and the light chains are identical; alternatively, the light chains are different (eg, V′-CL and V″-CL wherein V′=a first variable domain (eg, a VL, VH or dAb) that is capable (alone or paired with a V domain of the associated heavy chain) of binding a first antigen; and V″= a second variable domain (eg, a VL, VH or dAb) that is capable (alone or paired with a V domain of the associated heavy chain) of binding a second antigen). In an embodiment, a V domain of the first heavy chain is paired with a V domain of the first light chain and is comprised by a first VH/VL binding site that is capable of binding to a first antigen; and/or a V domain of the second heavy chain is paired with a V domain of the second light chain and is comprised by a first VH/VL binding site that is capable of binding to a second antigen. The antigens in this case are different, eg, different antigens of a virus, bacterium or cell.

Optionally each single variable domain is selected from a variable domain disclosed herein. Optionally, each VH/VL binding site is a VH/VL binding site comprised by an antibody disclosed herein (or encoded by VH and VL DNA sequences disclosed herein). Optionally, the antigen is a viral antigen (eg, a coronavirus or SARS-CoV or SARS-Cov-2 antigen, such as RBD or spike antigen) and each single variable domain is an anti-viral antigen variable domain (eg, nanobody or VH or VHH) disclosed herein, such as a variable domain that comprises the amino acid sequence of QB-GB (SEQ ID NO: 307), QB-DD, QB-BG or QB-FE). Optionally, the first and second antigen binding sites are different. Optionally, they are the same. Optionally, the first and second VH/VL sites are different. Optionally, they are the same. Optionally, the first VH/VL site is (i) a VH/VL binding site comprising a VH and a VL encoded by a VH DNA sequence and the cognate VL DNA sequence shown in Table 21(b), or (ii) a VH/VL binding site of any antibody disclosed in Table 21(a). For example, the VH and VL are encoded by VH-ICC and VL-ICC DNA sequences. Optionally, the second VH/VL site is (i) a VH/VL binding site comprising a VH and a VL encoded by a VH DNA sequence and the cognate VL DNA sequence shown in Table 21(b), or (ii) a VH/VL binding site of any antibody disclosed in Table 21(a). For example, the VH and VL are encoded by VH-IHG and VL-IHG DNA sequences. In one embodiment, the first and second VH/VL sites are the same. In another embodiment, they are different.

Optionally, a polypeptide herein (eg, a first; second; third; fourth; first and second; third and fourth; or first, second, third and fourth polypeptide) comprises in N-to C-terminal direction (i) a first antibody single variable domain, a second antibody single variable domain and TD; or a first antibody single variable domain, a second antibody single variable domain and Fc; or (iii) a first antibody single variable domain, a second antibody single variable domain and CH1. Each single variable domain is capable of binding to a respective antigen. Preferably, the antigens are different, although they may be the same. Optionally, the single variable domains are connected by a peptide linker (eg, a (G4S)n linker, wherein n=1, 2, 3, 4, 5, 6, 7, 8, 9, or 10, preferably 2 or 3). Optionally, the single variable domains are directly connected together.

Optionally, when the first and second polypeptides of the dimer are associated with the third and fourth polypeptides respectively, the first and third polypeptides comprise a respective variable domain, wherein the variable domains are comprised by a VH/VL pair that is capable of binding a first antigen; and wherein the second polypeptide comprises a first antibody single variable domain that is capable of binding a second antigen; and the fourth polypeptide comprises a second antibody variable domain that is capable of binding a third antigen. In an example, the first antigen is different from the second and third antigens. In an example, the second and third antigens are the same, or they may be different. Each single variable domain (dAb) may, for example, be a nanobody. Each single variable domain (dAb) may, for example, be a human dAb. In an embodiment, (i) the first polypeptide comprises a configuration of, in N- to C-terminal direction, V1-CH1-Hinge-CH2-CH3(with optional knob or first charged amino acid)-TD which is paired with the third polypeptide, wherein the third polypeptide comprises a configuration of, in N- to C-terminal direction, V2-CL, wherein V1 and V2 comprise a first VH/VL pair that is capable of binding to the first antigen (eg, wherein V1=VH and V2=VL; or V1=VL and V2=VH); and (ii) the second polypeptide comprises a configuration of, in N- to C-terminal direction, V3-CH1-Hinge-CH2-CH3(with optional hole that pairs with the knob; or optionally a second charged amino acid that pairs with the first charged amino acid)-TD, wherein the fourth polypeptide comprises a configuration of, in N- to C-terminal direction, V4-CL, wherein each of V3 and V4 is an antibody single variable domain that is capable of binding to a second and third antigen respectively. In an alternatiave, hinge is absent from the first and second polypeptides. Optionally, the first charge is a positive charge and the second charge is a negative charge. Optionally, the second charge is a positive charge and the first charge is a negative charge.

Optionally, wherein the dimer comprises the first and second polypeptides and a third (but not fourth) polypeptide, wherein the first polypeptide is associated with the third polypeptide, the first and third polypeptides comprise a respective variable domain, wherein the variable domains are comprised by a VH/VL pair that is capable of binding a first antigen; and wherein the second polypeptide comprises a first antibody single variable domain that is capable of binding a second antigen. In an example, the first antigen is different from the second antigen. Preferably, the second polypeptide is devoid of a CH1 domain. In an example, the first and second antigens are the same, or they may be different. The single variable domain (dAb) may, for example, be a nanobody. The single variable domain (dAb) may, for example, be a human dAb. In an embodiment, (i) the first polypeptide comprises a configuration of, in N- to C-terminal direction, V1-CH1-Hinge-CH2-CH3(with optional knob or first charged amino acid)-TD which is paired with the third polypeptide, wherein the third polypeptide comprises a configuration of, in N- to C-terminal direction, V2-CL, wherein V1 and V2 comprise a first VH/VL pair that is capable of binding to the first antigen (eg, wherein V1=VH and V2=VL; or V1=VL and V2=VH); and (ii) the second polypeptide comprises a configuration of, in N- to C-terminal direction, V3-Hinge-CH2-CH3 (with optional hole that pairs with the knob; or optionally a second charged amino acid that pairs with the first charged amino acid)-TD, wherein V3 is an antibody single variable domain that is capable of binding to the second antigen. In an alternatiave, hinge is absent from the first and second polypeptides. Optionally, the first charge is a positive charge and the second charge is a negative charge. Optionally, the second charge is a positive charge and the first charge is a negative charge. This arrangement, wherein the second polypeptide is devoid of a CH1, avoids the need for a polypeptide (such as a fourth polypeptide) that pairs with the second polypeptide. Thus, the third polypeptide will pair with the first polypeptide and undesirable pairing of the third polypeptide with the second polypeptide is avoided. This is useful for providing favourable yields of the desired dimer (comprising the first, second and third polypeptides with the third polypeptide paired with the first, but not the second polypeptide) and reduce contamination by the undesired configuration (comprising the first, second and third polypeptides with the third polypeptide paired with the first and the second polypeptides).

### Inhalable Compositions

A inhalable pharmaceutical composition (or dose of said composition) is provided which comprises particles of any multimer disclosed herein in the size range from 0.5 to 5.0 µm. For example, at least 60% (eg, 60-80% or 60-90%) of particles are in said size range. Optionally, at least 20% (eg, 20-40% or 20-35% or 20-30%) of particles are in the size range >4.7 µm (coarse particles), and optionally in the size range >4.7 µm but no more than 5.0 µm. Optionally, at least 50% (eg, at least 60%, 50-80%, 50-75%, 50-70% 50-65%) of particles are in the size range <4.7 µm (fine particles). Optionally, at least 15% (eg, at least 10%, at least 5%, at least 4, 3, 2 or 1%) of particles are in the size range <1.0 µm (ultra-fine particles). Preferably, the particles are nebulised particles. For example, the composition or dose is comprised by a nebuliser. For example, the composition or dose is comprised by an inhaler. For example, the composition or dose is obtainable by nebulising the multimer, eg, using an Aeroneb Solo™ nebuliser. The composition or dose comprises the multimer and a pharmaceutically acceptable carrier; such carriers for inhalable formulations will be familiar to the skilled addressee.

In a preferred example,
a) at least 20% (eg, 20-40% or 20-35% or 20-30%) of multimer particles are in the size range >4.7 µm, and optionally in the size range >4.7 µm but no more than 5.0 µm;
b) at least 50% (eg, at least 60%, 50-80%, 50-75%, 50-70% 50-65%) of multimer particles are in the size range <4.7 µm; and
c) at least 15% (eg, at least 10%, at least 5%, at least 4, 3, 2 or 1%) of multimer particles are in the size range <1.0 µm (ultra fine particles).

In a preferred example,
a) at least 20% of multimer particles are in the size range >4.7 µm, and optionally no more than 5.0 µm; and
b) at least 50% of multimer particles are in the size range <4.7 µm, and optionally at least 15% of multimer particles are in the size range <1.0 µm.

In a preferred example,
a) at least 20% of multimer particles are in the size range >4.7 µm, and optionally no more than 5.0 µm;
b) at least 50% of multimer particles are in the size range <4.7 µm; and
c) at least 15% of multimer particles are in the size range <1.0 µm.

The composition may comprise particles of the multimer and the median mass aerodynamic particle diameter (MMAD) is 2 to 4.5, eg, 2.5 to 4 or 3 to 3.5 µm.

In an example, the multimer comprises at least 4 copies of an antibody single variable domain, eg, a that specifically binds to a virus antigen, for example a RSV or SARS-CoV-2 antigen, eg, RBD or NTD. For example, the variable domain is CoVnb-112 (also called Nb-112 herein (SEQ ID NO: 288), and the virus antigen is a SARS-CoV-2 antigen), Nb11-59 (Novamab Biopharmaceuticals Co. Ltd, and the virus antigen is a SARS-CoV-2 antigen) or a variable domain of ALX-0171 (Ablynx BV, and the virus antigen is an RSV antigen).

Preferably, the polypeptide herein comprises the amino acid sequence of any one of SEQ ID NOs: 289 to 292. Preferably, the multimer herein comprises 4 copies of a polypeptide that comprises the amino acid sequence of any one of SEQ ID NOs: 289 to 292, or an amino acid sequence that is at least 80, 85, 90, 95, 96, 97, 98 or 99% identical to the amino acid sequence of any one of SEQ ID NOs: 289 to 292. The pharmaceutical composition herein may comprise such a multimer, eg, for administration to a human or animal subject for treating or preventing a lung condition. The lung condition may be a lung infection, such as a viral infection, or symptom thereof.

### Multimers for Resisting Sars-Cov-2 Mutation

The polypeptide described herein may, for example, comprise a SARS-CoV-2 antigen binding domain disclosed herein or a binding domain (eg, an antibody single variable domain) that competes with a SARS-CoV-2 antigen binding domain disclosed herein for binding to SARS-CoV-2 spike in an in vitro competition assay. In vitro competition may be determined by standard SPR or ELISA, for example. Any SPR herein is, for example, surface plasmon resonance (SPR) at 37° C. and pH 7.6.

The polypeptide described herein may, for example, comprise a SARS-CoV-2 antigen binding domain disclosed herein or a binding domain (eg, an antibody single variable domain) that binds to the same SARS-CoV-2 spike epitope (or an overlapping epitope) as a SARS-CoV-2 antigen binding domain disclosed herein.

The polypeptide described herein may, for example, comprise a binding domain that binds to the inner face of the RBD (receptor-binding domain) of SARS-CoV-2 spike.

The polypeptide described herein may, for example, comprise a binding domain that binds to the inner face of the RBD (receptor-binding domain) of SARS-CoV-2 spike that is in the up state. Similarly, the multimer herein may comprise copies of such a binding domain. The multimer described herein may, for example, bind to the inner face of the RBD (receptor-binding domain) of SARS-CoV-2 spike. The multimer described herein may, for example, bind to the inner face of the RBD (receptor-binding domain) of SARS-CoV-2 spike that is in the up state.

The multimer described herein may, for example, comprise copies of a SARS-CoV-2 antigen binding domain, wherein the multimer competes with a SARS-CoV-2 antigen binding domain-containing multimer (eg, Q185B see right-hand-side schematic in FIG. 14B, a tetramer of SEQ ID NO: 236) disclosed herein for binding to SARS-CoV-2 spike in an in vitro competition assay. In vitro competition may be determined by standard SPR or ELISA, for example. Any SPR herein is, for example, surface plasmon resonance (SPR) at 37° C. and pH 7.6.

The multimer described herein may, for example, comprise copies of a SARS-CoV-2 antigen binding domain, wherein the multimer binds to the same SARS-CoV-2 spike epitope (or an overlapping epitope) as a SARS-CoV-2 antigen binding domain-containing multimer (eg, Q185B see right-hand-side schematic in FIG. 14B, a tetramer of SEQ ID NO: 236) disclosed herein.

The polypeptide described herein may, for example, comprise binding domain QB-GB or a binding domain (eg, an antibody single variable domain) that competes with QB-GB for binding to SARS-CoV-2 spike in an in vitro competition assay. In vitro competition may be determined by standard SPR or ELISA, for example. Any SPR herein is, for example, surface plasmon resonance (SPR) at 37° C. and pH 7.6.

The polypeptide described herein may, for example, comprise binding domain QB-GB or a binding domain (eg, an antibody single variable domain) that binds to the same SARS-CoV-2 spike epitope (or an overlapping epitope) as QB-GB.

The polypeptide described herein may, for example, comprise a binding domain that binds to the inner face of the RBD (receptor-binding domain) of SARS-CoV-2 spike.

The polypeptide described herein may, for example, comprise a binding domain that binds to the inner face of the RBD (receptor-binding domain) of SARS-CoV-2 spike that is in the up state.

Similarly, the multimer herein may comprise copies of such a binding domain.

As explained in Example 37 Quad multimers that have such features have been found to be highly advantageous and may be more resistant to receptor-driven selection pressure associated with SARS-Cov-2 mutation.

Furthermore, there is provided a method of producing a polypeptide multimer, the method comprising multimerising first, second, third and fourth copies of a polypeptide (eg, any polypeptide disclosed herein) that comprises at least one copy of an SARS-CoV-2 antigen binding domain (eg, QB-GB or a binding domain (eg, an antibody single variable domain) that competes with QB-GB for binding to SARS-CoV-2 spike in an in vitro competition assay, and optionally formulating the multimer in a pharmaceutical composition for administration (eg, injected or pulmonary administration) to a human or animal subject to treat or prevent a coronavirus (preferably, SARS-CoV-2) infection.

Furthermore, there is provided a method of producing a polypeptide multimer, the method comprising multimerising first, second, third and fourth copies of a polypeptide (eg, any polypeptide disclosed herein) that comprises at least one copy of an SARS-CoV-2 antigen binding domain (such as an antibody variable domain) that binds to the same SARS-CoV-2 spike epitope (or an overlapping epitope) as QB-GB, and optionally formulating the multimer in a pharmaceutical composition for administration (eg, injected or pulmonary administration) to a human or animal subject to treat or prevent a coronavirus (preferably, SARS-CoV-2) infection.

Furthermore, there is provided a method of producing a polypeptide multimer, the method comprising multimerising first, second, third and fourth copies of a polypeptide (eg, any polypeptide disclosed herein) that comprises at least one copy of an SARS-CoV-2 antigen binding domain (such as an antibody variable domain) that binds to the inner face of the RBD (receptor-binding domain) of SARS-CoV-2 spike, and optionally formulating the multimer in a pharmaceutical composition for administration (eg, injected or pulmonary administration) to a human or animal subject to treat or prevent a coronavirus (preferably, SARS-CoV-2) infection.

Furthermore, there is provided a method of producing a polypeptide multimer, the method comprising multimerising first, second, third and fourth copies of a polypeptide (eg, any polypeptide disclosed herein) that comprises at least one copy of an SARS-CoV-2 antigen binding domain (such as an antibody variable domain) that binds to the inner face of the RBD (receptor-binding domain) of SARS-CoV-2 spike that is in the up state, and optionally formulating the multimer in a pharmaceutical composition for administration (eg, injected or pulmonary administration) to a human or animal subject to treat or prevent a coronavirus (preferably, SARS-CoV-2) infection.

The binding domain may be an immunoglobulin domain (eg, an antibody variable domain or single variable domain (dAb or nanboby), or any other type of binding site or domain disclosed herein.

Any example, configuration, Aspect, Concept, Clause or Paragraph or disclosure herein is combinable with any feature of any further example, configuration, Aspect, Concept, Clause or Paragraph or disclosure herein.

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications (including US equivalents of all mentioned patent applications and patents) are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

### Example 1: Generation of Tetravalent and Octavalent Soluble Heterodimeric NY-ESO-1 TCR Molecules

This example demonstrates a method for generating tetravalent and octavalent soluble heterodimeric TCR molecules referred to as ts-NY-ESO-1 TCR and os-NY-ESO-1 TCR respectively. These formats overcome the problems associated with solubility and avidity for cognate ligand at the target site.

To exemplify ts-NY-ESO-1 TCR and os-NY-ESO-1 TCR as stable and soluble molecules, TCR αβ variable sequences with high affinity for NY-ESO-1 together with immunoglobulin constant domains and the NHR2 tetramerisation domain are used in this example.

The high-affinity NY-ESO TCR αβ chains (composing of TCR Vα-Cα and Vβ-Cβ respectively) specific for SLLMWITQC-HLA-A^{∗}0201 used in this example is as reported in WO 2005/113595 A2 with the inclusion of a signal peptide sequence (MGWSCIILFLVATATGVHS). To aid protein purification, a histidine tag was incorporated to the C-terminus of NHR2 domain.

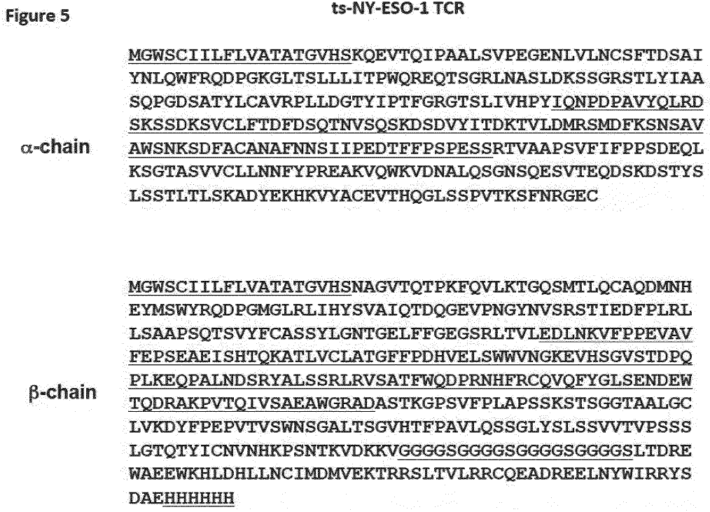

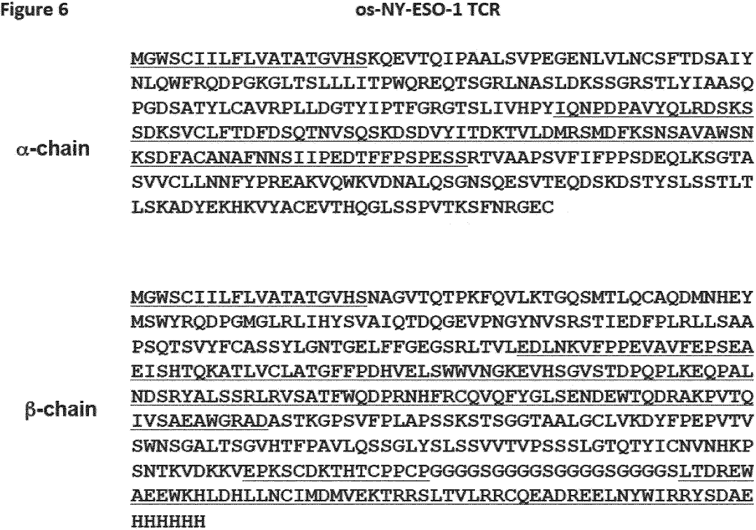

DNA constructs encoding components of ts-NY-ESO-1 TCR and os-NY-ESO-1 TCR are synthetically constructed as a two-vector system to allow for their soluble expression and functional assembly in mammalian cells. A schematic representation of the two assembled TCR chains (α and β chains) whose DNA sequences are synthesized for cloning into the expression vector are shown in FIGS. 3 and 4 and their amino acid sequences are shown in FIGS. 5 and 6.

The pTT5 vector system allows for high-level transient production of recombinant proteins in suspension-adapted HEK293 EBNA cells (Zhang et al., 2009). It contains origin of replication (oriP) that is recognized by the viral protein Epstein-Barr Nuclear Antigen 1 (EBNA-1), which together with the host cell replication factor mediates episomal replication of the DNA plasmid allowing enhanced expression of recombinant protein. Therefore the pTT5 expression vector is selected for cloning the components for the ts-NY-ESO-1 TCR and os-NY-ESO-1 TCR molecules.

Synthesized DNA fragments containing the TCR αβ chains are digested with restriction enzymes at the restriction sites (RS) (FastDigest, Fermantas) and the DNA separated out on a 1% agarose gel. The correct size DNA fragments is excised and the DNA purified using Qiagen gel extraction kit. The pTT5 vector was also digested with the same restriction enzymes and the linearized plasmid DNA is purified from excised agarose gel. The digested TCR αβ chains is cloned into the digested pTT5 vector to give four expression vectors (pTT5-ts-NY-ESO-1-TCRα, pTT5-ts-ESO-1-TCRβ, pTT5-os-NY-ESO-1-TCRα and pTT5-os-ESO-1-TCRβ).

### Expression of Tetravalent and Octavalent Soluble NY-ESO TCR

Functional expression of ts-NY-ESO-1 TCR and os-NY-ESO-1 TCR is carried out in suspension-adapted HEK293 EBNA cells. HEK293-EBNA cells are cultured in serum-free Dulbecco’s Modified Eagle Medium (DMEM, high glucose (4.5 g/L) with 2 mM L-glutamine) at 37° C., 5% CO₂ and 95% humidity.

For each transfection, HEK293-EBNA cells (3 × 10⁷ cells) are freshly seeded into 250 mL Erlenmeyer shaker Flask (Corning) from ~60% confluent cells. Transfections are carried out using FreeStyle MAX cationic lipid base reagent (Life Technologies) according to the supplier’s guidelines. For expression of ts-NY-ESO-1 TCR, 37.5 µg of total plasmid DNA (18.75 µg plasmid DNA each of pTT5-ts-NY-ESO-1-TCRα and pTT5-ts-ESO-1-TCRβ vectors are used or varying amounts of the two expression plasmids) are used per transfection. Similarly for expression of os-NY-ESO-1 TCR, 18.75 µg plasmid DNA each of pTT5-os-NY-ESO-1-TCRα and pTT5-os-ESO-1-TCRβ isare used for transfection. Following transfection, cells were recovered in fresh medium and cultivated at 37° C. with 5% CO₂ in an orbital shaker at 110 rpm for between 4-8 days. Smaller scale transfections are done similarly in 6 well or 24 well plates.

### Analysis of Expressed Soluble eTCR²-BiTE

The ts-NY-ESO-1 TCR and os-NY-ESO-1 TCR protein molecules secreted into the supernatant are analyzed either directly by sodium dodecyl sulfate (SDS) polyacrylamide gel electrophoresis (PAGE) or after protein purification. Protein samples and standards are prepared under both reducing and non-reducing conditions. SDS-PAGE was performed using cast mini gels for protein electrophoresis in a Mini-PROTEAN Tetra cell electrophoresis system (Bio-Rad). Coomassie blue dye was used to stain proteins in SDS-PAGE gel.

### Purification of ts-NY-ESO-1 TCR and os-NY-ESO-1 TCR Protein Molecules

Soluble ts-NY-ESO-1 TCR and os-NY-ESO-1 TCR from cell supernatant are purified in two steps. In the first step immobilized metal affinity chromatography (IMAC) are used with nitrilotriacetic acid (NTA) agarose resin loaded with nickel (HisPur Ni-NTA Superflow agarose -Thermo fisher). The binding and washing buffer consists of Tris-buffer saline (TBS) at pH7.2 containing low concentration of imidazole (10-25 mM). Elution and recovery of the His-tagged ts-NY-ESO-1 TCR and os-NY-ESO-1 TCR from the IMAC column are achieved by washing with high concentration of imidazole (>200 mM). The eluted protein fractions are analysed by SDS-PAGE and the fractions containing the protein of interest are pooled. The pooled protein fraction is used directly in binding assays or further purified in a second step involving size-exclusion chromatography (SEC). Superdex 200 increase prepacked column (Gelifesciences) are used to separate out monomer, oligomer and any aggregated forms of the target protein.

### Surface Plasmon Resonance

The specific binding and affinity analysis of ts-NY-ESO-1 TCR and os-NY-ESO-1 TCR to its pMHC is performed using BIAcore. Briefly, the purified Histidine-tagged ts-NY-ESO-1 TCR and os-NY-ESO-1 TCR proteins are captured onto sensor surface via Ni²⁺ chelation of nitrilotriacetic acid (NTA). Varying concentration of the analyte solution containing NY-ESO pep_{(SLLMWITQV)}-MHC (ProImmune)is injected and the binding signals were monitored

### Example 2: Generation of Tetravalent and Octavalent Soluble NY-ESO TCR-IL2 Fusion Molecule

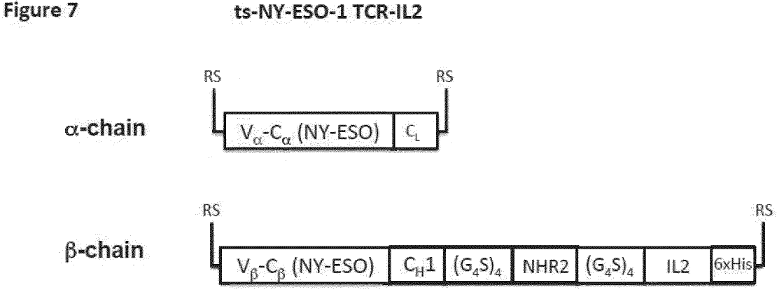

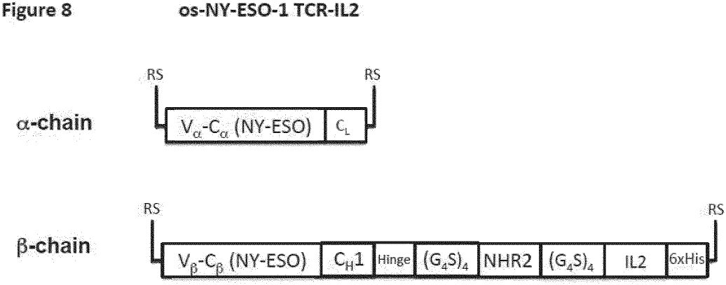

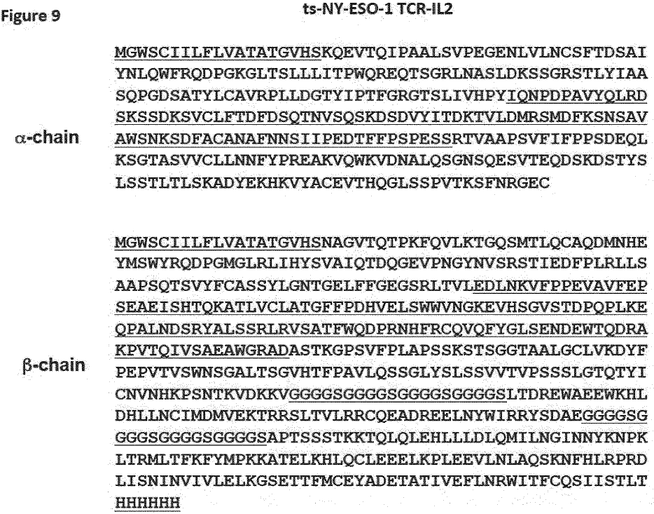

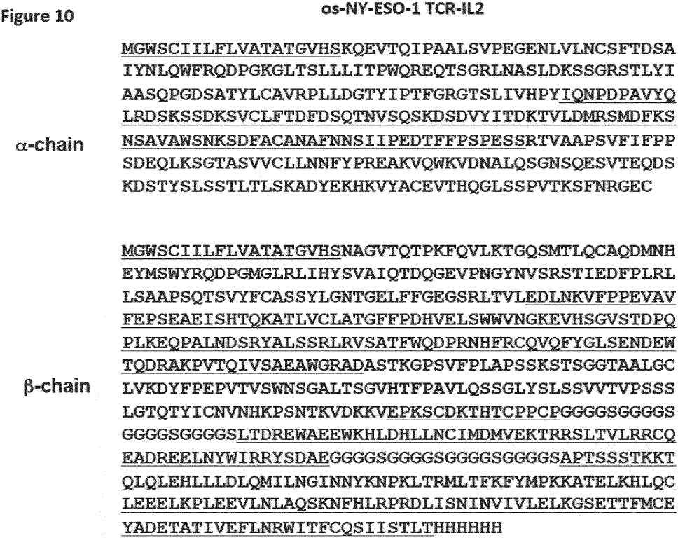

The DNA encoding the domains required for expressing ts-NY-ESO-1 TCR-IL2 and os-NY-ESO-1 TCR-IL2 protein complexes are synthesized and cloned into the expression vector pTT5 as described above. A schematic representation of the domains within the TCR αβ chains for ts-NY-ESO-1 TCR-IL2 and os-NY-ESO-1 TCR-IL2 are shown in FIGS. 7 and 8 and the amino acids sequences are shown in FIGS. 9 and 10. Expression, purification and characterization of ts-NY-ESO-1 TCR-IL2 and os-NY-ESO-1 TCR-IL2 are carried out as described above.

### Example 3: High Yield Tetramer Secretion

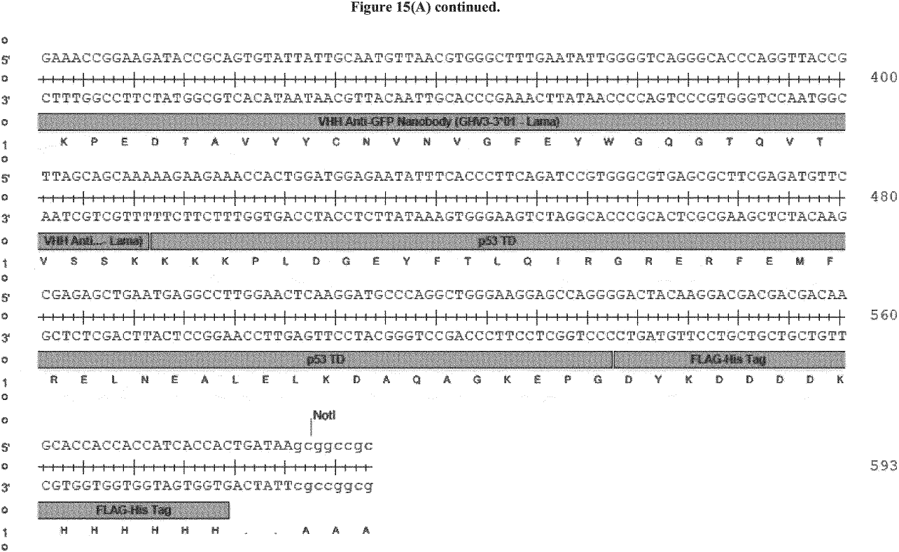

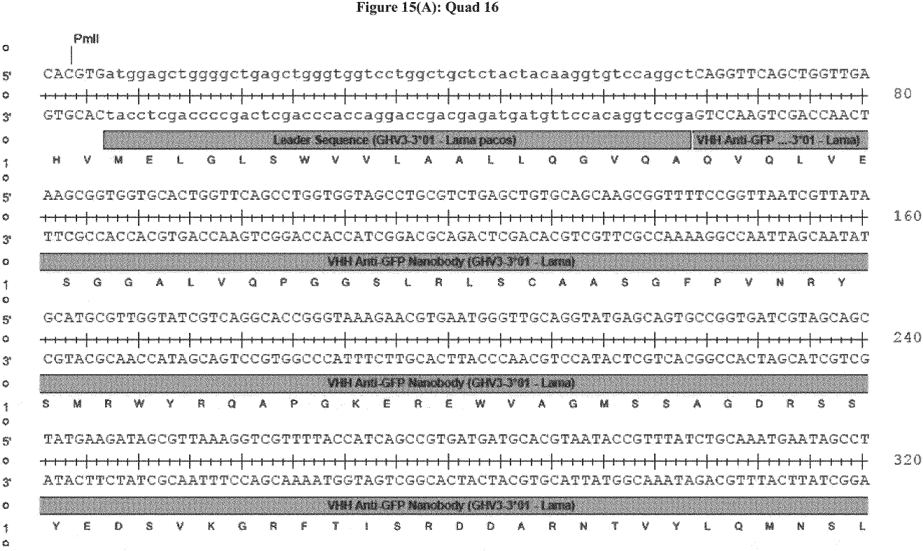

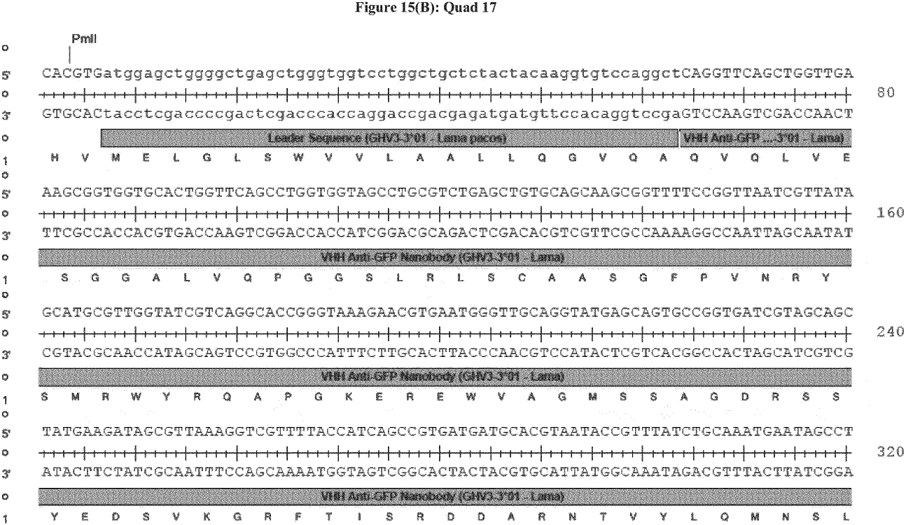

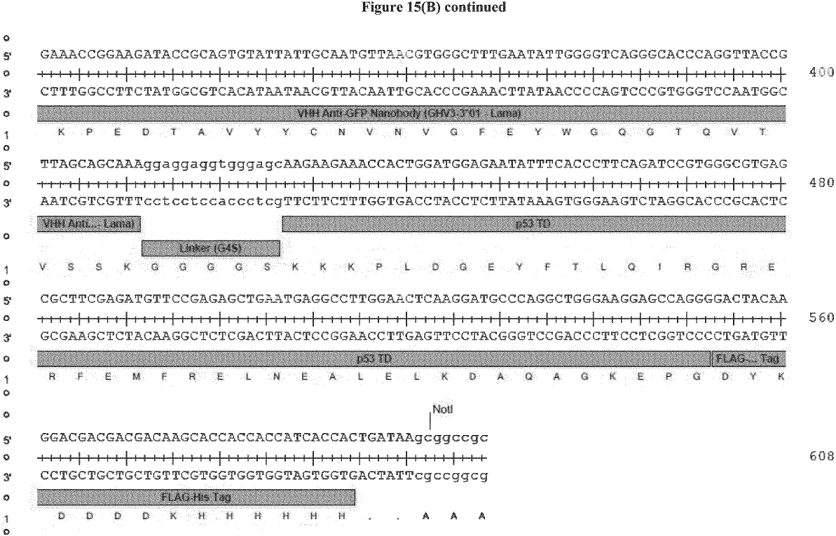

Briefly, using conventional genetic engineering techniques, a HEK293-T cell line was made that encodes Quad 16 (FIGS. 14 and 15) and another HEK293-T cell line was made that encodes Quad 17 (FIGS. 14 and 15).

Protein expression took place and protein was secreted from the cell lines. Samples of the medium in which the cells were incubated were subjected to PAGE under denaturing conditions (SDS-PAGE) or under native conditions (no SDS). The former was further under reduced conditions (using mercaptoethanol), whereas the latter was not.

The reduced gel showed a distinct banding (FIG. 16) at the expected monomer size. Surprisingly, the unreduced, native gel showed no detectable banding at the monomer, dimer or trimer size, but instead heavy banding was seen at the tetramer size indicating that a very high yield of tetramer had been obtained, and this was confirmed by SEC to be of high purity.

For Quad 16, the tetramer peak from SEC was run on SDS-PAGE and the obtained band was cut out for mass spectrometry. The data were obtained with trypsin digests and p53 was detected in 100% of the protein. This was conclusive evidence that the secreted Quad 16 was multimerised

### Example 4: Intracellular Protein Expression of Extracellular Portion of TCR Fused to NHR2 TD

### Expression Vector

All DNA fragments were synthesized and cloned into the expression vector, pEF/myc/cyto (Invitrogen) by Twist Bioscience (California). Schematics and sequences of the synthesized DNA fragments and Quad polypeptides are shown in FIG. 21, and the sequence tables herein.

### DNA Preparation

Lyophilised plasmid DNA synthesized by Twist Bioscience, were resuspended with MQ water to a concentration of 50 ng/µl. 50 ng of DNA was transformed into 50 µl of competent DH5α cells using a conventional heat shock method. The cells were plated on LB agar plates containing 100 µg/mL ampicillin and grown overnight at 37° C. Individual colonies were picked and grown overnight at 37° C., 220 rpm. The DNA was purified from the cells using the QIAprep Spin Miniprep Kit, according to the manufacturers instructions (Qiagen).

### Transfections in HEK293T Cell

Briefly, HEK293T cells were maintained in high glucose DMEM supplemented with 10% FBS and Pen/Strep. Cells were seeded at 6 × 10⁵ cells per well of a 6-well plate in 2 ml media and were allowed to adhere overnight at 37° C., 5% CO₂. 7.5 µl of Lipofectamine 2000 was diluted in 150 µl of OptiMem and incubated at room temperature for 5 mins. Plasmid DNA (2.5 µg) was diluted in 150 µl of OptiMem. Diluted DNA was combined with the diluted Lipofectamine 2000, mixed gently and incubated at R.T. for 20 mins. The 300 µl of complexes were added to one well of the 6-well plates. When analysis required the media to be serum free, the media was aspirated and replaced with CD293 media 6 hours post-transfection. The cells were incubated for 48 hours at 37° C., 5% CO₂ prior to analysis.

Accordingly, different formats of TCR-linked NHR2 tetramerisation domain (TD) constructs (Quads) were transfected into HEK293T cells. Quads 3 & 4 resembling a TCR tetravalent format (structure schematically represented in FIGS. 1 & 3) and Quads 12 & 13 resembling a TCR octavalent format (Structure schematically represented in FIGS. 2 & 4) were transfected for protein expression analysis. Protein samples were prepared from transfected HEK293T cells as follows to check for intracellularly expressed protein. Briefly cells were washed once with 2 ml PBS, which was subsequently aspirated. 150 µl of Trypsin-EDTA (0.05%) was added to each well and the cells were incubated at R.T. for ~1 min. The plate was tapped to lift any strongly adhering cells. 850 µl of media was added to each well to inactivate the trypsin. The cells were transferred to a 1.5 ml eppendorf and spun at 1,000 rpm for 5 mins. The supernatant was aspirated and the pellets stored on ice. The cells were resuspended in 400 µl cell lysis buffer (10 mM Tris pH 7.5, 1% SDS) containing Protease Inhibitor Cocktail Set III (Calbiochem), diluted 1/200. The samples were vortexed vigorously and incubated on ice for 20 mins. The cells were sonicated using a Branson Ultrasonics Sonifier™ (Thermo Fisher Scientific). The amplitude was set to 30% and the cells were sonicated for a total of 24 seconds (6 secs on, 3 secs off ×4). The total protein concentration was quantified using the Pierce

BCA Protein Assay Kit™, according to the manufacturer’s instructions. 100 µg was diluted with MQ water to give a volume of 80 µl. 20 µl of 5× SDS loading buffer was then added giving samples of 1 mg/ml. Samples were incubated at 95° C. for 5 min prior to SDS-PAGE and Western blot analysis.

Protein samples were separated on SDS-PAGE under denaturing condition. Typically, 25 µg of whole cell lysate (25 µl) were loaded on to the gel for Western blot analysis. 5 µl of PageRule Prestained 10-180 kDa Protein Ladder was loaded into the gel alongside the protein samples. The gels were run in Tris-Glycine buffer containing 0.1% SDS. A constant voltage of 150 volts was used and the gels were run for ~70 mins until the dye front has migrated fµlly.

SDS-PAGE (15% Bis-Tris) gels were prepared using the following resolving and stacking gels.

Resolving Gel:
5 ml 30% Bis-Acrylamide
2.6 ml 1.5 M Tris (pH 8.8)
50 µl 20% SDS
100 µl 10% APS
10 µl TEMED
2.2 ml MQ Water

Stacking Gel:
0.75 ml 30% Bis-Acrylamide
1.25 ml 1.5 M Tris (pH 8.8)
25 µl 20% SDS
50 µl 10% APS
5 µl TEMED
2.9 ml MQ Water

Western blotting was performed for the specific and sensitive detection of protein expression of TCR-NHR2 TD fusion proteins from Quads 3, 4, 12 and 13. Proteins separated out on SDS-PAGE were transferred onto Amersham Hybond™ 0.45 µM PVDF membrane as follows. Briefly, Amersham Hybond 0.45 µM PVDF membrane was activated with MeOH for ~1 min and rinsed with transfer buffer (25 mM Tris, 190 mM Glycine, 20% MeOH) before use. The sponge, filter paper, gel, membrane, filter paper, sponge stack was prepared and placed in the cassette for transfer. Transfer was carried out on ice at 280 mA for 75 mins. The membrane was incubated for ~2 hours in blocking buffer (TBST, 5% milk powder). The membrane was washed briefly with TBST before being incubated at 4° C. overnight with anti-human IgG HRP (Thermo, 31410) diluted 1/2500 in TBST, 1% milk powder. The membrane was washed thoroughly (three washes of TBST, 15 mins each) before being developed using the Pierce ECL Western Blotting Substrate.

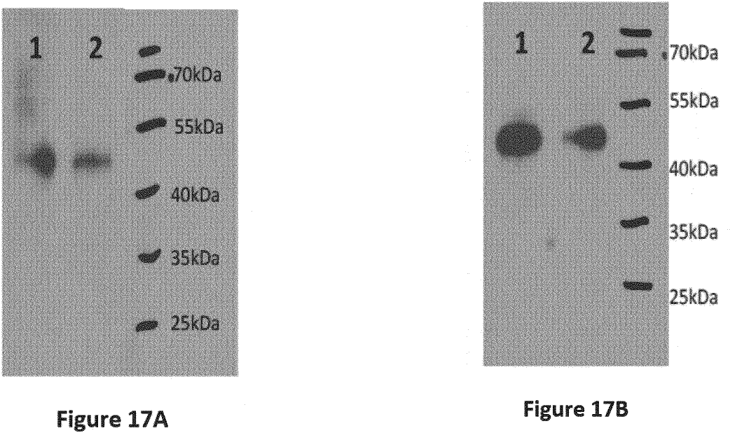

Using anti-human IgG detection antibody to probe Western blots, specific protein band at the expected molecular weight can be detected from samples prepared from Quads 3 (46.1 kDa), 4 (46.4 kDa), 12 (47.8 kDa) and 13 (48.1 kDa) (FIGS. 17A and 17B). These data confirm intracellular protein expression of TCR-NHR2 TD fusion proteins in HEK293T cells.

For all of the Quads analysed, a clear single band can be detected indicating TRVβ-TRCβ-IgG1-CH1 (+/- IgG hinge domain) fusions with the NHR2 TD are stable. These expression data also confirm the possibility of assembling tetravalent (Quads 3 & 4) and octavalent (Quads 12 & 13) molecules as exemplified in this example.

The difference between Quads 3 and 4 is the presence of a small peptide linker (G4S) located between the IgG1 CH1 domain and NHR2 TD. This is also true for Quads 12 and 13 where Q13 contains a peptide linker between the IgG1 CH1 domain and NHR2 TD. From the expression data, it can be seen the peptide linker does not effect protein expression. However, it may be desirable to include a peptide linker to aid antigen binding and or stabilizing the multimerisation complex in these TCR-NHR2 TD formats.

### Example 5: Soluble Protein Expression of Extracellular Portion of TCR Fused to NHR2 TD

TCR-NHR2 TD fusion proteins were shown in Example 4 to be expressed intracellularly in HEK293T cells. Here again Quads 3, 4, 12 and 13 were used to demonstrate soluble expression of these fusion proteins. As described above, Quads 3, 4, 12 and 13 were transfected into HEK293T cells and soluble proteins from the cell supernatant were concentrated. Briefly, the media was harvested 48 hours post-transfection and centrifuged at 2,000 rpm for 5 mins to remove any cells or debris. Typically, 500 µl of media was concentrated to 100 µl using Amicon™ Ultra 0.5 Centrifugal Units with a MWCO of 10 kDa. 25 µl of 5× SDS loading buffer was added to the sample, which was then incubated at 95° C. for 5 mins prior to gel/Western blot analysis. Concentrated protein samples were separated out on SDS-PAGE gel and transferred onto Amersham Hybond 0.45 µM PVDF membrane. Western blotting and protein detection was done using anti-human IgG HRP using the methods described above.

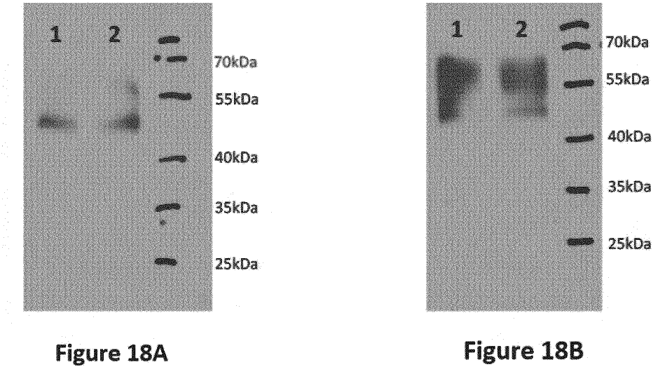

Protein samples concentrated and prepared from cell supernatants show specific protein band at the expected molecular weight on Western blots corresponding to Quads 3 (46.1 kDa), 4 (46.4 kDa), 12 (47.8 kDa) and 13 (48.1 kDa) (FIGS. 18A and 18B). The Western blot expression data unequivocally shows soluble expression of TCR-NHR2 TD fusion proteins in HEK293T. These data are the first report demonstrating soluble expression of TCR-NHR2 TD fusion proteins expressed in eukaryotic cells such as HEK293T cells.

Detection of soluble protein expression from both tetrameric (Quads 3 & 4) and octameric (Quads 12 & 13) TCR-NHR2 TD formats highlights the potential applicability of NHR2 TD in a broad setting. Use of NHR2 TD fusion molecules could be used for the preparation of therapeutic molecules and protein molecules for use in diagnostics and imaging.

### Example 6: Intracellular Protein Expression of Antibody Fragments Fused to NHR2 TD

To further exemplify the versatility of NHR2 TD, several different antibody fragment formats fused to NHR2 TD were constructed for testing their expression in HEK293T cells.

Quads 14 and 15 contain an antibody VH domain fused to NHR2 TD either with or without a peptide linker located between the VH and NHR2 TD as schematically depicted in FIGS. 11 and 21. The VH domain in Quads 14 and 15 are specific for GFP (green fluorescent protein). Several other versions of this format were also constructed and tested with VH specific for therapeutically useful drug targets. Sequences of the binding domains are listed in Table 4. Some of these include Quad 34 (specific for TNFα), Quad 38 (specific for VEGF), Quad 40 (specific for EGFR) and Quad 44 (specific for CD38).

Quads 38 and 44 were further developed to include an additional binding arm with the inclusion of a second VH domain specific for EGFR and CD138 respectively yielding Quads 42 and 46. Quads 42 and 46 represent bispecific molecules with the capability to multimerise via the NHR2 TD domain to form bispecific tetramers.

In another example, an effector molecule (human IL2) was linked to the C-terminus of Quads 14 & 15 resulting in Quads 18 and 19, whereby the VH-NHR2-IL2 molecule is tetravalent and bifunctional.

In another example, antibody Fab fragment (VH-CH1) was linked to NHR2 TD (Quads 23 and 24) and as schematically depicted in FIG. 12. Quads 23 and 24 represent tetravalent Fab molecules when co-expressed or mixed and assembled in-vitro with a second chain containing immunoglobulin light chain (e.g. Quad 25).

In yet another example, a human IgG1 hinge domain was included to Quads 23 and 24, which is referred to as Quads 26 and 27 and as schematically depicted in FIG. 13. Quads 26 and 27 represent octavalent Fab molecules when co-expressed or mixed and assembled in-vitro with a second chain containing immunoglobulin light chain domains (e.g. Quad 25).

The following Quad vectors, Quads 14, 15, 18, 19, 23, 24, 26, 27, 34, 38, 40, 42, 44 and 46 all of which are His-tagged were transfected in HEK293T cells. Protein samples were prepared from whole-cell extracts as described above, separated out on SDS-PAGE and transferred onto Amersham Hybond 0.45 µM PVDF membrane. Specific protein expression were probed using anti-His HRP (Sigma, A7058) diluted 1/2500 in TBST, 1% milk powder.

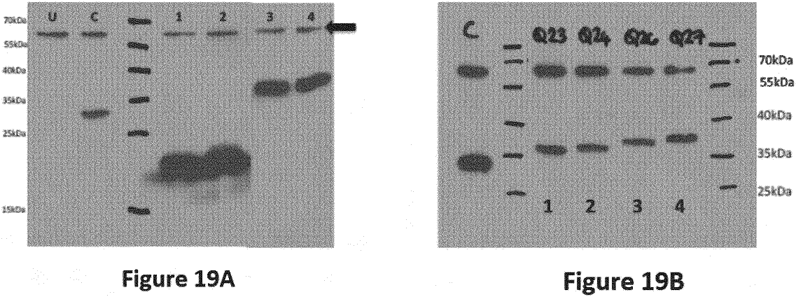

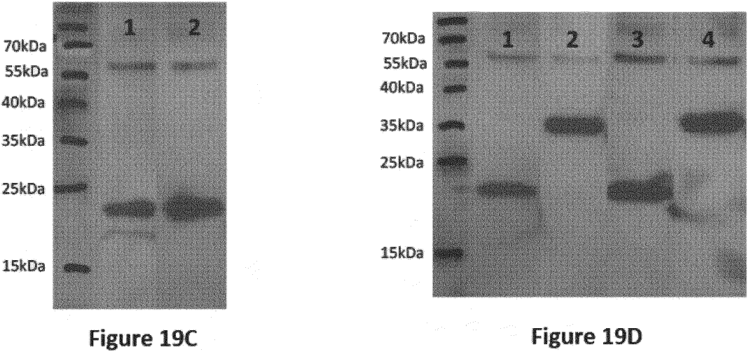

Specific protein expression in whole cell extracts could be detected for all the different antibody-NHR2 TD fusion proteins tested using Quads 14, 15, 18, 19, 23, 24, 26, 27, 34, 38, 40, 42, 44 and 46 (FIGS. 19A-D). Interestingly, Quads 18 and 19 containing an effector domain (IL2) fused to the C-terminus of NHR2 TD domain, in addition to the VH binding domain fused to the N-terminus of NHR2 TD showed good protein expression.

Expression of Quads 23 and 24 polypeptides highlights the potential to use NHR2 TD to form tetravalent antibody Fab molecules when co-expressed or mixed in-vitro with a partner soluble Quad molecule (e.g. Quad 25). Similarly expression of Quads 26 and 27, which include human IgG1 hinge domain highlight the potential to use NHR2 TD to form octavalent antibody Fab molecules when co-expressed or mixed in-vitro with a partner soluble second partner chain (e.g. Quad 25).

Quads 42 and 46 bispecific molecules containing an additional VH domain fused to the C-terminus of NHR2 TD domain also showed good protein expression. These data highlights the versatility of the NHR2 TD domain and its ability to be fused to different binding and effector molecules for developing a vast array of protein formats. The data also suggest it is possible to fuse protein molecules to both the N-terminus and C-terminus of NHR2 TD, which allows for the development of bispecific multivalent protein molecules.

### Example 7: Multivalent Assembly of Antibody Fragments Fused to NHR2 TD

NHR2 TD is responsible for the oligomerisation of ETO into a tetrameric complex. Using the NHR2 TD domain, it is possible to fuse binding domains and effector molecules to the N-terminus or C-terminus or both N- and C-terminus without effecting expression as shown in examples 4-6. Binding domains could be TCR variable and constant domains, antibody and antibody fragments or effector molecules such as IL2. It is also possible to express proteins in a soluble format when fused to NHR2 TD (FIG. 18) despite NHR2 TD being a part of an intracellularly expressed protein where in nature it is only expressed inside the cell.

To demonstrate whether NHR2 TD retains its potential to oligomerise once it is fused to a binding domain, Quads 14 and 15 were expressed in HEK293T cells and protein samples were prepared from whole cell extracts as described above. Protein samples were separated out on PAGE gel under denaturing and non-denaturing (native) conditions. Native gels were prepared using the protocol described above, but without SDS. Proteins from PAGE gels were transferred onto Amersham Hybond 0.45 µM PVDF membrane. Specific protein expression was probed with anti-human IgG HRP detection antibody.

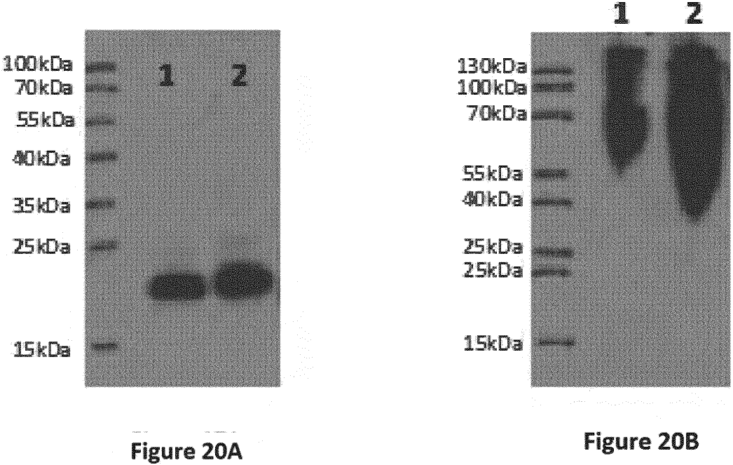

As expected under denaturing conditions, expression of VH-NHR2 TD from Quads 14 and 15 can be seen as a monomer where a specific protein band can be detected at the expected molecular weight (22 and 22.3 kDa) (FIG. 20A). Under non-denaturing and thus native conditions, interestingly no monomer or dimer of VH-NHR2 TD from Quads 14 and 15 can be detected (FIG. 20B). Only a high molecular weight protein band believed to be tetramers of VH-NHR2 TD from Quads 14 and 15 can be detected. The assembly of tetramers appears to be highly efficient and pure judging by the protein intensity and the absence of any detectable monomers and dimers of Quads 14 and 15.

Together with the data in examples 4-7, there is conclusive evidence NHR2 TD is highly versatile allowing fusion of various protein binding domains and effector molecules. NHR2 TD allows soluble expression of proteins from eukaryotic cells such as HEK293T cells and they form highly stable and pure tetrameric molecules.

### FURTHER EXAMPLES

### Methods

### Expression Vectors

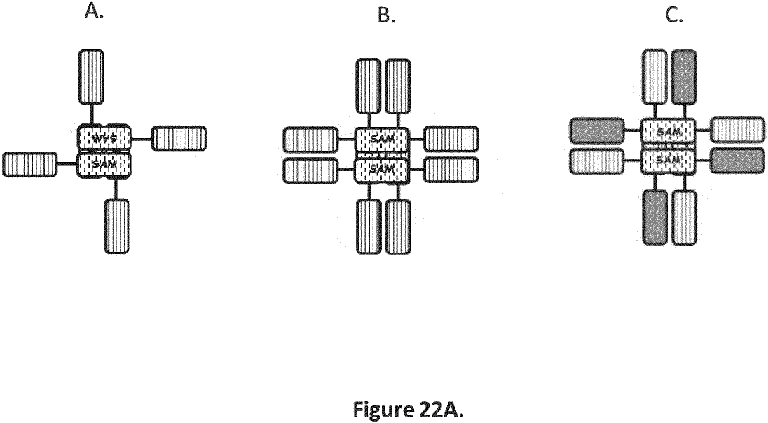

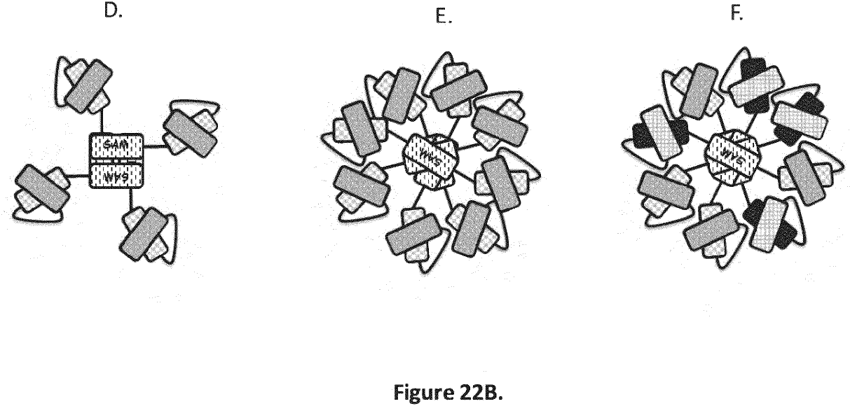

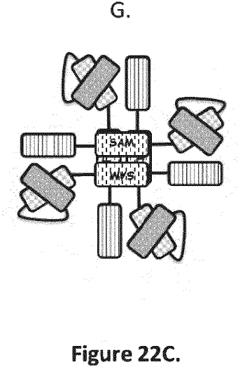

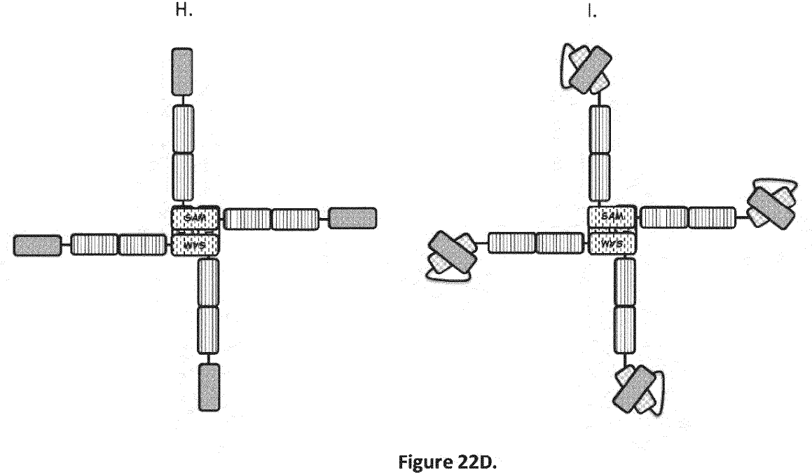

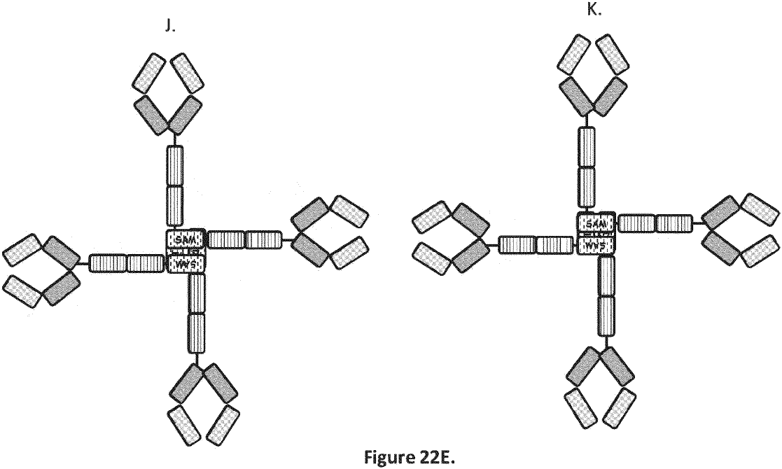

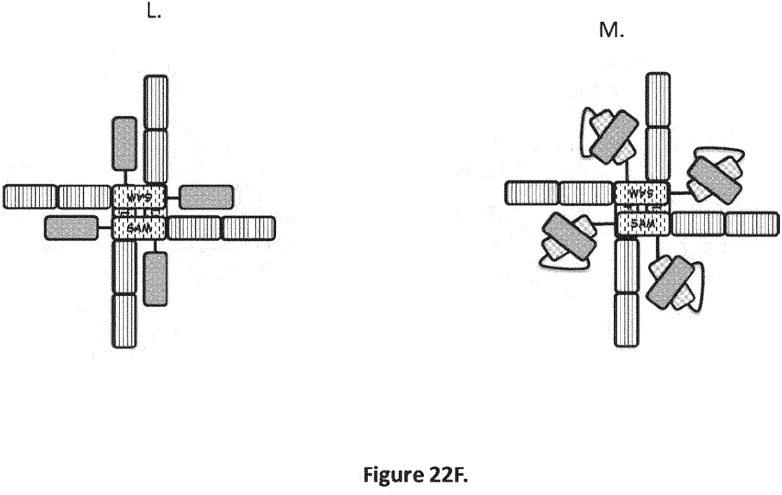

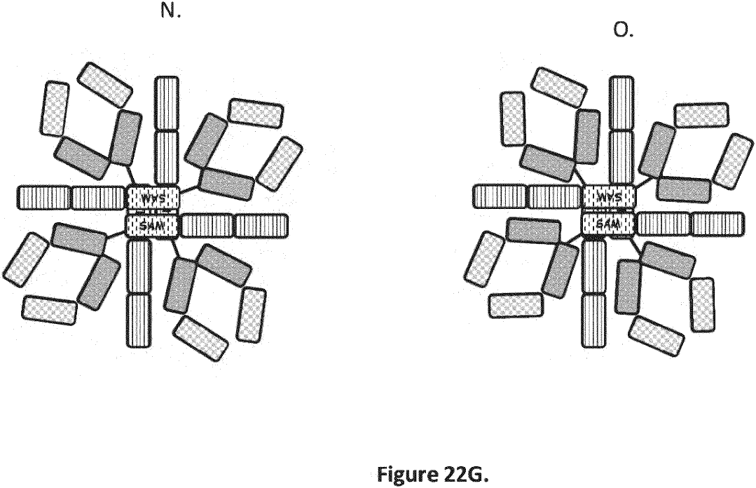

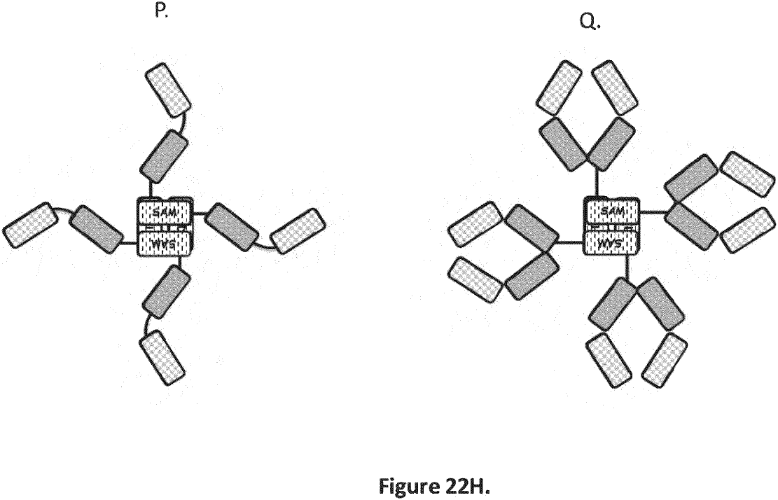

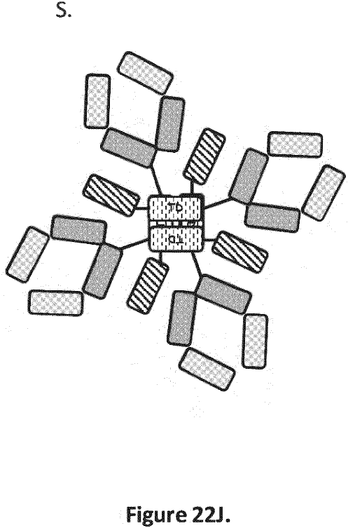

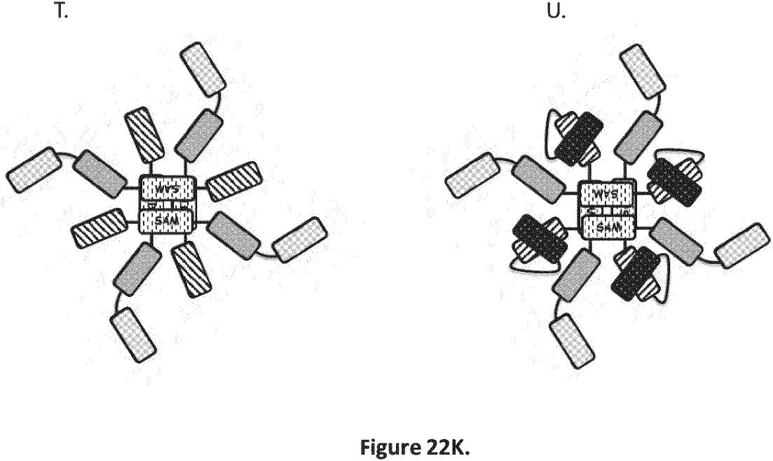

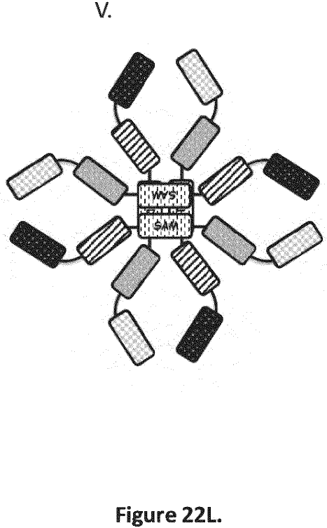

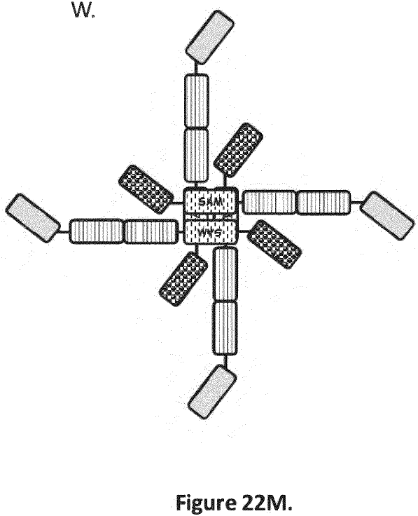

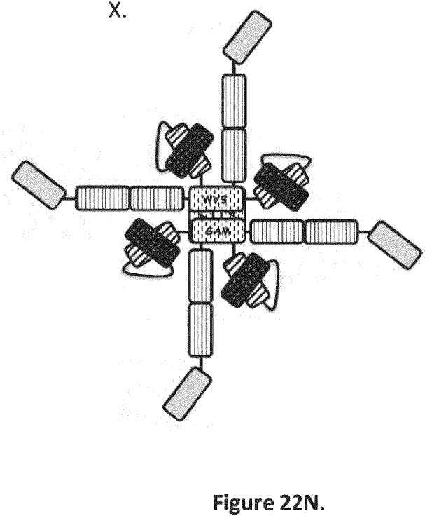

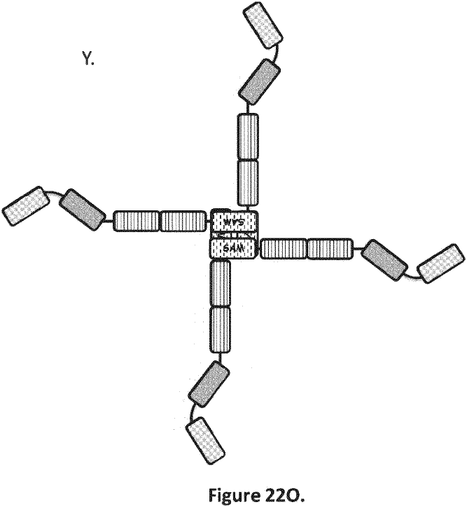

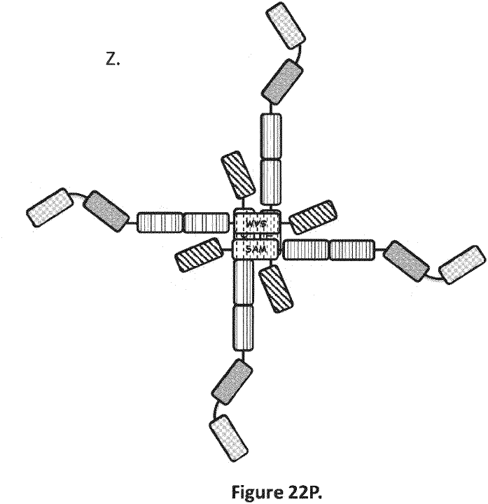

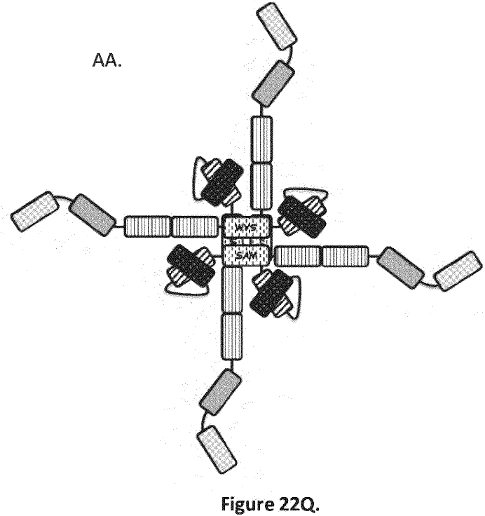

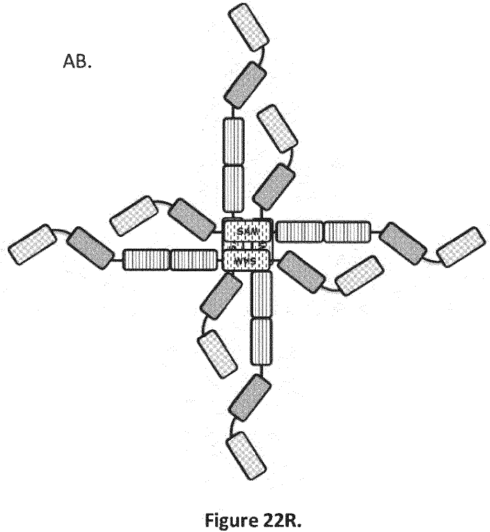

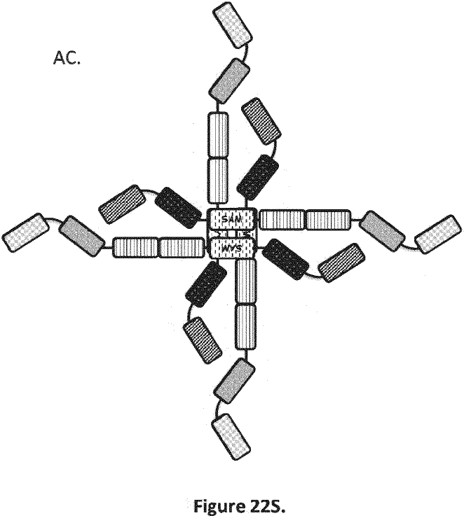

All DNA fragments were synthesized by Twist Bioscience (California) and cloned into the expression vector, pEF/myc/cyto (Invitrogen). Schematics and sequences of the synthesized DNA fragments are shown in FIG. 22 and Tables 8 -10, respectively.

### Plasmid DNA Preparation

Lyophilised plasmid DNA synthesized by Twist Bioscience, were resuspended with MQ water to a concentration of 50 ng/µl. Competent E. coli DH5α cells were transformed with 50 ng of DNA using a conventional heat shock method. Transformed cells were plated on LB agar plates containing 100 µg/mL ampicillin and grown overnight at 37° C. Individual colonies were picked and grown in LB broth overnight at 37° C., 220 rpm. Plasmid DNA were purified from the cells using Qiagen plasmid extraction kits, according to the manufacturer’s instructions (Qiagen).

### Expression of Quad Proteins in Expi293F Cells

Expi293F™ cells (Thermo Fisher Scientific) were cultured in Expi293™ Expression Medium (Thermo Fisher Scientific) according to the manufacturer’s recommendations. The only exception was that 5% CO₂ was added directly to the flasks when the cells were split and non-vented caps were used.

Two methods involving different transfection reagents were utilised for protein expression. The methods for 30 ml cultures are described below and the protocol was adapted to either scaled up or down according to the experimental requirements.

For PEI transfections the cells were counted one day prior to transfection using a NC-3000™ (ChemoMetec) and were diluted to 1.5 × 10⁶ cells/ml using Expi293™ Expression Medium. The cells were cultured in 5% CO₂ at 37° C., 125 rpm overnight. The following day the cells were counted, spun down for 5 minutes at 1000 rpm and resuspended at 2 × 10⁶ cells/ml in 30 ml of fresh media. 33 ug of plasmid DNA was added to 900 ul media and 90 ul of PEI Max (Polysciences Inc.) was added to 900 ul media. The DNA and transfection reagent samples were mixed and incubated at room temperature for 15 minutes. The DNA/transfection reagent mixture was added to the cells, which were cultured as before and incubated for a further 72 hrs.

For transfections with Expifectamine™ 293 Reagent (Thermo Fisher Scientific) the cells were also diluted to 1.5 × 10⁶ cells/ml in Expi293™ Expression Medium one day prior to transfection. On the day of transfection the cells were centrifuged and resuspended at 2.5 × 10⁶ cells/ml in 30 ml of fresh media. Two tubes containing 1.5 ml of Gibco™ Opti-MEM™ (Thermo Fisher Scientific) were prepared. 30 ug of plasmid DNA was added to one tube and 80 ul of Expifectamine was added to the other. The solutions were mixed and incubated at room temperature for 30 minutes. The DNA-transfection reagent complex was added to the cells, which were cultured in 5% CO₂ at 37° C., 125 rpm. Following 16-18 hrs incubation, transfection enhancers 1 and 2 were added to the cells according to the manufacturers protocol. The cells were incubated for a further 96 hours.

### Purification of His-Tagged Proteins

The cells were harvested by centrifugation for 10 minutes at 4000 rpm. The ~30 ml supernatant was filtered through a 0.22 µm filter and diluted to 50 ml with binding buffer (50 mM HEPES, pH 7,4, 250 mM NaCl, 20 mM imidazole) containing Complete™ EDTA-free protease inhibitors (Roche) to facilitate binding to the column. A 1 ml HisTrap™ HP column (GE Healthcare) was connected to an AKTA Start (GE Healthcare) and pre-equilibrated with binding buffer. The protein-containing media was loaded onto the column using a flow rate of 1 ml/min. The column was washed with >10 CV of binding buffer before the protein was eluted using a 20-300 mM imidazole gradient over 12 ml. 0.5 ml fractions were collected and analysed by SDS-PAGE. Protein containing fractions were pooled and concentrated using Amicon® Ultra centrifugal filter units (Millipore).

Following affinity chromatography the proteins were either snap frozen and stored at -80° C., dialysed into an alternative buffer for a specific application of gel filtrated to assess the molecular weight of the various Quad formats. For the latter analyses, protein samples were concentrated to 1.5-2 ml and gel filtrated on a Superdex 75 16/600 column (GE Healthcare) using 10 mM HEPES, pH 7.4, 250 mM NaCl.

### Sds-Page

Purified proteins were analysed by separating out on SDS-PAGE under denaturing condition. Typically, 1-2 µg of purified protein were loaded per lane on SDS-PAGE gel. The gels were run in Tris-Glycine buffer containing 0.1% SDS. A constant voltage of 150 volts was used and the gels were run for ~70 mins until the dye front has migrated fully.

SDS-PAGE (15% Bis-Tris) gels were prepared using the following resolving and stacking gels.

Resolving Gel:
5 ml 30% Bis-Acrylamide
2.6 ml 1.5 M Tris (pH 8.8)
50 µl 20% SDS
100 µl 10% APS
10 µl TEMED
2.2 ml MQ Water

Stacking Gel:
0.75 ml 30% Bis-Acrylamide
1.25 ml 1.5 M Tris (pH 8.8)
25 µl 20% SDS
50 µl 10% APS
5 µl TEMED
2.9 ml MQ Water

### General Direct Binding ELISA Assay

The potential of the purified Quad proteins to bind its target protein was confirmed by directing binding ELISA. Briefly, high binding 96 well plates (Corning) were used for coating recombinant target protein (1-5 ug/ml diluted in PBS or as indicated), which were typically stored at 4° C. overnight. Plates are then washed 3 times with 200 ul wash buffer (PBS + 0.1% Tween) and blocked using 200 ul blocking buffer (PBS + 1% BSA) for 1 hour at room temperature. Purified protein samples are typically serially diluted in dilution buffer (PBS + 0.1% BSA) and 100ul/well is added. Samples are incubated at room temperature for 1 hour after which the plate is washed again 3 times using 200 ul wash buffer. Anti-His HRP (Abcam) Detection antibody diluted according to the manufacturer recommendation is added and incubated at room temperature for 1 hour. The plate is washed for the final time using 3×200 ul wash buffer and 50 ul pre-warmed detection reagent (TMB -Sigma) is added per well and the plate incubated in the dark for 10-30 mins. The reaction is stopped by adding 25 ul/well of 1 M sulfuric acid. The absorbance at 450 nm was read using a CLARIOstar microplate reader (BMG Labtech).

### Example 8: Expression of Monospecific Tetravalent dAb Quad

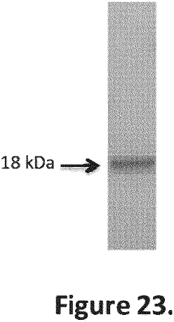

A multimer format (“Quad” format) where a single domain of an antibody variable fragment (dAb) (VH or VL either Vκ or Vλ) fused to p53 tetramerisation domain is exemplified in this example. The dAb VH sequence for anti-IL17A (sequence adapted from WO 2010/142551 A2) was engineered into a tetravalent dAb Quad format (Quad 57) (Seq ID: 146). Expression vector for Quad 57 was synthesized by Twist Bioscience and expressed in HEK293 cells as described above. Secreted Quad 57 protein was collected from cell supernatant and purified using HisTrap HP column. To demonstrate soluble expression and purity of Quad 57, a small portion of the purified protein (1.5 ug) was separated out on SDS-PAGE gel (FIG. 23). A pure protein could be detected at the expected size (18 kDa) corresponding to Quad 57 confirming soluble expression of a tetravalent dAb Quad protein. A schematic representation of a tetravalent monospecific dAb Quad is shown in FIG. 22-A (ie, embodiment A of FIG. 22A).

### Example 9: Expression and Binding Analysis of Bispecific Tetravalent dAb Quad

A bispecific tetravalent dAb Quad is exemplified in this example where two different dAb binding domains are linked to p53 tetramerisation domain via the N- and C-terminus and as schematically represented in FIG. 22-C(ie, embodiment C of FIG. 22A).

In a specific example of a bispecific tetravalent dAb Quad, an anti-TNFa dAb VH binding domain from Ozoralizumab was linked to the N-terminus of p53 tetramerisation domain and an anti-IL17A dAb VH binding domain (sequence adapted from WO2010/142551 A2) was linked to the C-terminus of the p53 tetramerisation domain (Quad 54) (Seq ID: 143). Although in this specific example both the dAb binding domains were VH’s, the dAb binding domains could be Vκ or Vλ or a combination of the different dAb formats.

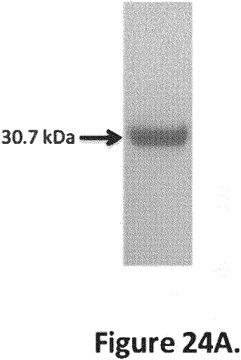

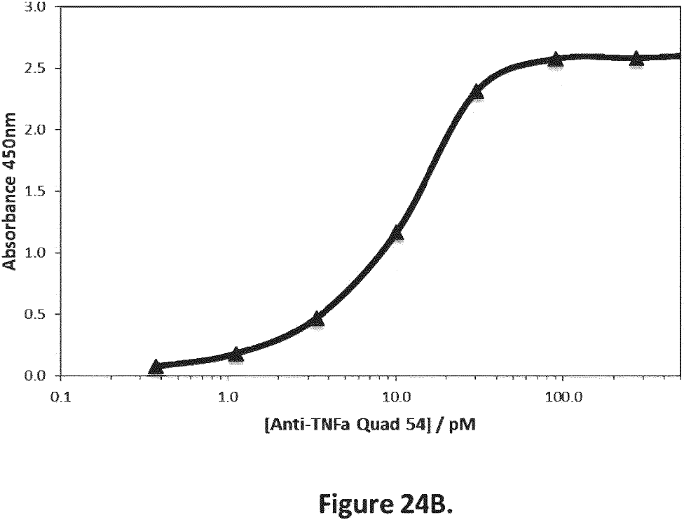

To demonstrate whether such a bispecific tetravalent dAb Quad format could be expressed as a soluble protein, expression construct containing dual anti-TNFa and anti-IL17A dAb binding domains were synthesized as a Quad format and expressed in HEK293 cells. Following protein purification from culture supernatant using HisTrap HP column, ~1.5 ug of the purified protein was separated out on SDS-PAGE gel (FIG. 24A). From the SDS-PAGE gel, a pure product at the expected size (30.7 kDa) could be seen confirming expression of soluble bispecific tetravalent dAb Quad.

To further exemplify the functionality of such bispecific tetravalent anti-TNFa x anti-IL17A dAb Quad, direct binding ELISA assay was performed. High protein binding 96 well plates (coming) were coated with 1 ug/ml recombinant human TNFa protein (Abcam) and direct binding ELISA assay was performed with serially diluted Quad 54 protein using the method described above. A typical sigmoidal dose response curve was yielded from Quad 54 direct binding ELISA with recombinant human TNFa protein with a half-maximal binding at the low pM range (~10 pM) (FIG. 24B). This confirms that not only can bispecific tetravalent anti-TNFa x anti-IL17A dAb Quads could be expressed as soluble proteins, they are also functional in that they can bind their target protein with high binding strength. The high binding strength is likely a measure of the increased avidity gained through tetravalent binding and this highlights an important feature of Quad multivalent molecules.

### Example 10: Expression, Binding and Functional Analysis of Monospecific Tetravalent scFv Quads

In this example, scFv binding domains for two different targets were selected and linked separately to the N-terminus of p53 tetramerisation domain to generate monospecific tetravalent scFv Quads as schematically represented in FIG. 22-D(ie, embodiment D of FIG. 22B).

In one example the scFv binding domain was an anti-TNFa where the VH and Vκ sequence was adapted from Humira into a scFv Quad format. To analyse whether the presence of a peptide linker effected the expression and binding of the Quad molecule to its target protein, in this example the anti-TNFa scFv binding domain was linked to the N-terminus of p53 tetramerisation domain either via a (G4S)3 peptide linker (Quad 63) (Seq ID: 147) or without a peptide linker (Quad 51) (Seq ID: 139).

In another example, the scFv binding domain was an anti-CD20 adapted from Wu et al., (Wu et al. 2001). The anti-CD20 scFv binding domain was engineered into a tetravalent Quad format by linking the binding domain directly to the N-terminus of p53 tetramerisation domain without a peptide linker (Quad 53 Tet) (Seq ID: 141).

All tetravalent scFv Quads were transfected into HEK293 cells and soluble protein from the culture supernatant were purified using HisTrap™ HP column as described above. A small amount of the purified protein (~1.5 ug) was separated out on SDS-PAGE gels to confirm expression and purity.

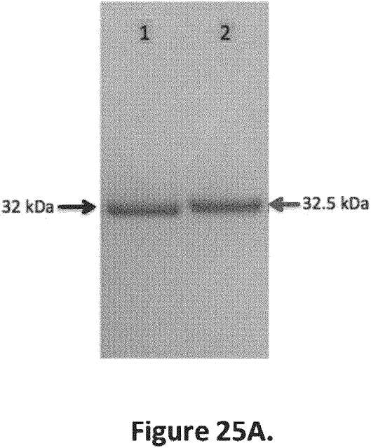

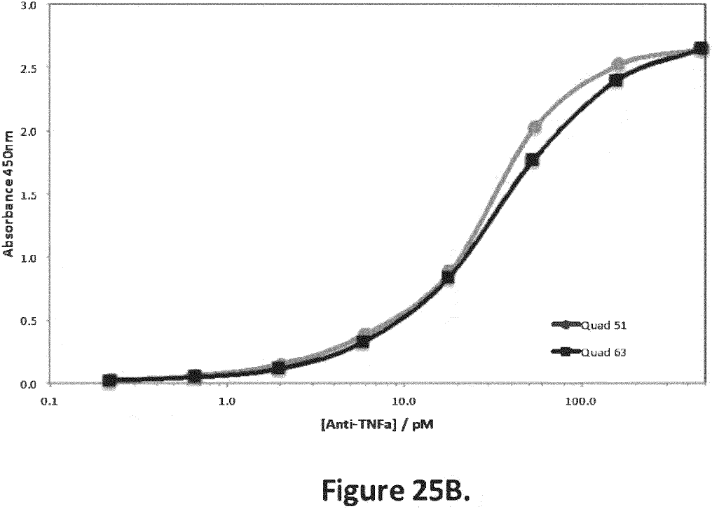

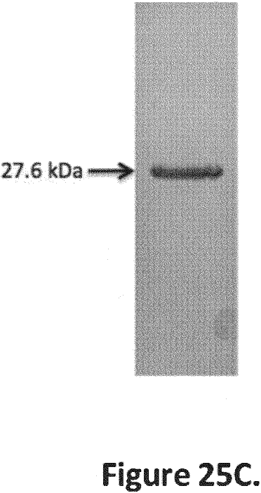

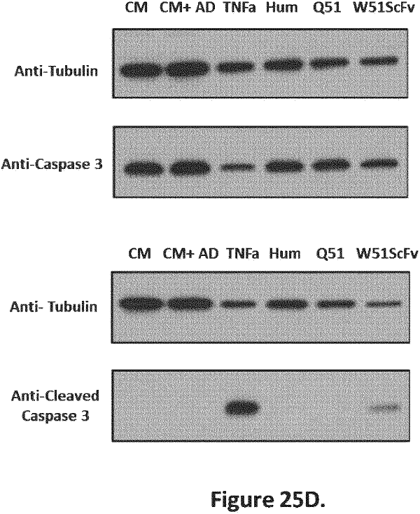

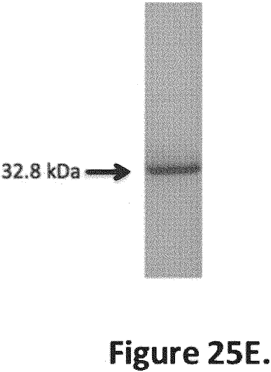

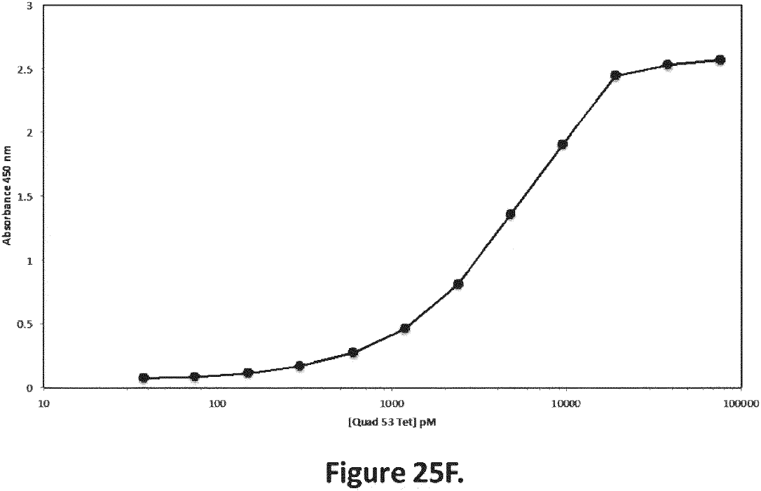

Anti-TNFa scFv Quads (Quads 51 & 63) were found to express well as soluble protein and the presence or absence of the peptide linker joining the scFv to the N-terminus of p53 tetramerisation domain did not appear to effect expression (FIG. 25A). Furthermore the purity of the secreted soluble Quad proteins appears to be highly pure (>99%). To characterize these Quads further, both Quad 51 and Quad 63 were analysed in a direct binding ELISA assay using recombinant human TNFa (Abcam). High binding 96 well plates (Costar) was coated with 1 ug/ml of human TNFa protein and ELISA binding assay was performed using serially diluted Quad 51 and Quad 63 proteins as described above. A dose-dependent increase in binding was observed for both Quad 51 and Quad 63 confirming these anti-TNFa scFv’s were assembled correctly and their native binding functionality were retained in this Quad format. Furthermore, the binding profile for Quad 51 and Quad 63 was found to be highly similar with both Quads having a half-maximal binding concentration in the low pM range (~30 pM) (FIG. 25B). Combinedly, these data confirm the presence or absence of the peptide linker does not effect protein expression or Quad binding strength to their target protein.

To further functionally characterize the activity of anti-TNFa Quad 51 molecule to neutralize TNFa, a cell-based assay was performed using WEHI-13VAR cells (ATCC), which is highly sensitive to TNFa. The bioassay using WEHI cells was set-up as described below. A monovalent anti-TNFa (W51ScFv) was included in the assay as a control (Seq ID: 150) and Humira was used as a positive control. W51ScFv was generated by modifying Quad 51 where the p53 tetramerisation domain was removed. Expression of W51ScFV was confirmed by SDS-PAGE (FIG. 25C).

Briefly, WEHI-13VAR cells were seeded at 1 × 10⁴ cells per well in a 96-well plate in RPMI-1640, 10% FBS and incubated overnight at 37° C., 5% CO₂. The media was aspirated from the cells and replaced with media containing 2 ug/ml actinomycin D, 0.1 ng/ml recombinant human TNFα (ab9649, Abcam) and 0-2400 pM Q51, Q35, W51ScFV and Humira. The samples were set up in quadruplicate with no TNFα and no antibody controls. The cells were incubated under standard culture conditions for a further 20-22 hours.

To assess cell viability, ATP generated by metabolically active cells was quantified using the CellTiterGlo Luminescent Cell Viability Assay (Promega) according to the manufacturers’ instructions. Luminescent signals were measured using a CLARIOstar microplate reader (BMG Labtech). The luminescence signals obtained from the compound treated cells were normalised against the media on controls. The effective dose (ED) at which 50% of the WEHI cells retained viability was calculated and the ED50 for Quad 51, Humira and W51ScFV is summarised in Table 11.

As expected, Quad 51 having four anti-TNFa binding domains was found to be most effective at neutralising the cytotoxic effect of recombinant human TNFa protein on WEHI cells compared to Humira, which has two binding domains for TNFa. The W51ScFv control having only a single binding domain for TNFa had the highest ED50. The increasing valency of the anti-TNFa molecules for TNFa correlated inversely with a decrease in ED50 values. These data highlights the enhanced functional potency of Quad molecules with increasing valency and this aligns with the general concept of avidity verses potency as reported by numerous studies (Alam et al. 2018; Rudnick & Adams 2009; Adams et al. 2006; Bru nker et al. 2016). Furthermore, the increase in avidity of Quad molecules can be used to drive selectively of tumour associated antigens that are highly expressed on tumour cells and thus limit the on-target off-tumour effect on healthy cells as reported in an example outlined by Slaga et al (Slaga et al. 2018).

To further demonstrate the ability of anti-TNFa Quad 51 to block TNFa induced activation of Caspase 3, Western blot analysis were performed alongside Humira and W51ScFv as controls. Briefly, WEHI-13VAR cells were seeded at 2.5 × 10⁶ cells per well in a 6-well plate in culture media (RPMI-1640, 10% FBS) and incubated overnight. The media was replaced with media containing 2 ug/ml actinomycin D, 1 ng/ml recombinant human TNFα and 500 pM of Quad 51, W51ScFV and Humira. Culture media only and culture media containing 2 ug/ml actinomycin D were included as controls. The cells were incubated with TNFα and +/- anti-TNFa molecules for 10 hours.

The cells were lysed using RIPA buffer (50 mM Tris, pH 8.0, 150 mM NaCl, 1% Triton X-100, 0.5% sodium deoxycholate, 0.1% SDS) containing 1 mM DTT and Complete™ EDTA-free protease inhibitor (Roche). Cell lysates were sonicated using a Bioruptor® Pico Sonication System (Diagenode) and the protein concentration of each sample was quantified using the Pierce BCA Protein Assay Kit (Thermo Fisher Scientific). 10 ug of protein was electrophoresed on a 12% Bis-Tris gel and bands were subsequently transferred to Amersham™ Hybond® P 0.45 µm PVDF membranes (GE Healthcare). The membranes were blocked with 10%-BSA-TBST or 5%-milk-TBST before being incubated overnight at 4° C. with appropriate antibodies (anti Caspase-3 (1/1000, CST, 9665) and anti-Cleaved Caspase-3 (1/100, CST, 9664). The membranes were washed with TBST and incubated with anti-rabbit IgG HRP-linked (1/2500, CST, 7074S) secondary antibody for 2 hours at room temperature. Following thorough washing, the membranes were developed using Pierce™ ECL Western Blotting Substrate (Thermo Fisher Scientific) and CL-XPosure™ films (Thermo Fisher Scientific).

From the Western blots, it can be seen that Quad 51 can effectively neutralise TNFa mediated activation of Caspase-3 with no detectable cleaved Caspase-3, similar to Humira (FIG. 25D). Whereas the monovalent W51ScFv anti-TNFa control molecule with high ED50 value, is not able to completely neutralize TNFa mediated activation of Caspase-3 and this is indicated by the presence of cleaved Caspase-3 in this sample. These data confirm that the mechanism of action of Quad 51 is through a signaling mechanism similar to Humira™. In a second example, the anti-CD20 scFv Quad (Quad 53 Tet) was expressed in HEK293 cells and following purification, protein expression analysis and protein binding assays were performed. SDS-PAGE analysis confirmed soluble expression of Quad 53 Tet as a highly pure protein (>99%) (FIG. 25E). ELISA binding assay using recombinant human CD20 protein (Abcam) using serially diluted Quad 53 Tet protein confirmed a dose-dependent increase in binding. The ability of the anti-CD20 scFv Quad to retain binding to CD20 once again confirms correct assembly of this Quad format into a functional molecule (FIG. 25F).

### Example 11: Expression and Binding Analysis of Monospecific Octavalent scFv Quad

In this example, the valency of the scFv binding domain was increased from four (tetravalent) to eight (octavalent). This was achieved by linking an scFv to both N- and C-terminus of the p53 tetramerisation domain as schematically represented in FIG. 22-E.

As described in example 10, the anti-CD20 scFv binding domain was used in this example to construct a monospecific octavalent anti-CD20 scFv Quad (Quad 53 Oct) (Seq ID: 142). In this specific example, scFv’s were linked to the N- and C-terminus of the p53 tetramerisation domain without peptide linkers, however, in other examples of this format, peptide linkers could be introduced at either or both ends to aid flexibility of the binding domain.

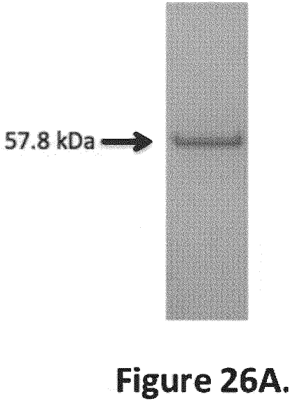

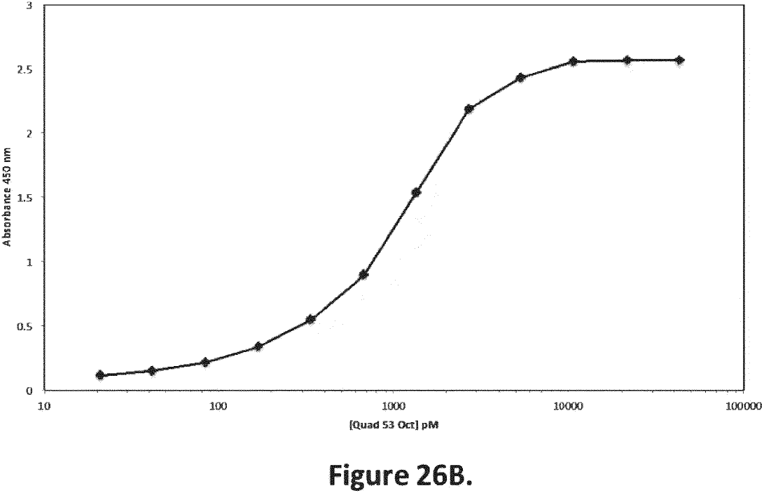

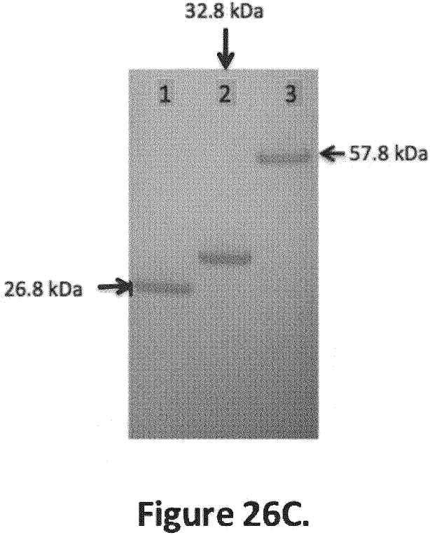

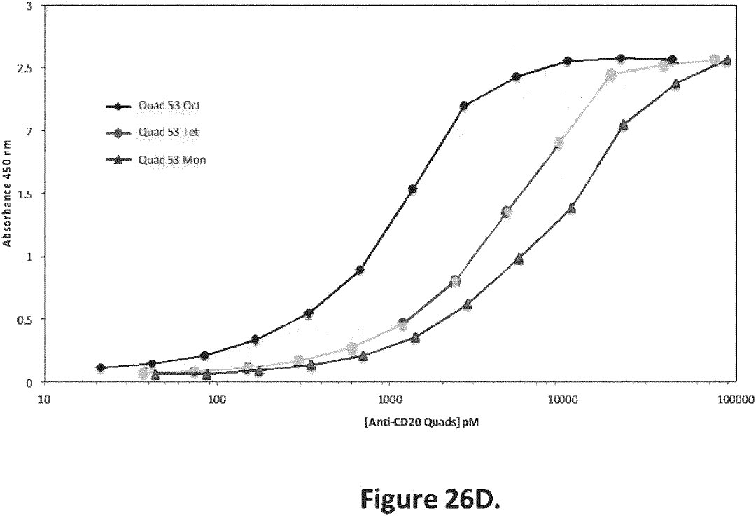

Following expression and purification of Quad 53 Oct protein from culture supernatant, protein expression analysis and ELISA binding assays were performed. Protein separated out on SDS-PAGE gel confirmed soluble expression of Quad 53 Oct with high purity (>99%) (FIG. 26A). ELISA binding assay was also performed using recombinant CD20 and serially diluted Quad 53 Oct protein (FIG. 26B). A dose-depended increase in binding could be seen confirming correct assembly of anti-CD20 scFv in an octavalent format.

To analyse the effect of increasing valency on target protein binding, scFv anti-CD20 without the tetramerisation domain was constructed to act as a monovalent control (Quad 53 Mon) (Seq ID: 140). Following protein expression and purification, all three Quad 53 anti-CD20 scFv molecules (monovalent, tetravalent and octavalent) were separated out side-by-side on a SDS-PAGE gel (FIG. 26C) and ELISA binding assay was performed (FIG. 26D). With all three Quad 53 molecules, a dose-dependent increase in binding could be seen and the overall increase in binding strength was reflected by the increase in the valency. The octavalent anti-CD20 scFv version was found to have the highest overall binding strength, followed by the tetravalent and then the monovalent version. It is noteworthy, due to the nature of direct binding ELISA where the target protein is densely coated onto 96 well plates resulting in rapid Quad 53 binding saturation to CD20, the true avidity (total binding strength) can not be accurately determined using this method. Despite these limitations of direct binding ELISA, a representative increase in binding could still be observed with increasing valency as seen in FIG. 26D.

### Example 12: Expression and Binding Analysis of Bispecific Tetravalent scFv Quad

In this example, two different scFv binding domains with specificity for two different target proteins were linked to the p53 tetramerisation domain via the N- and C-terminus to give a bispecific tetravalent scFv Quad as exemplified schematically in FIG. 22-F.

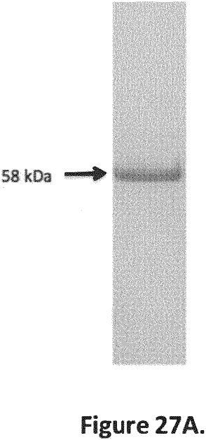

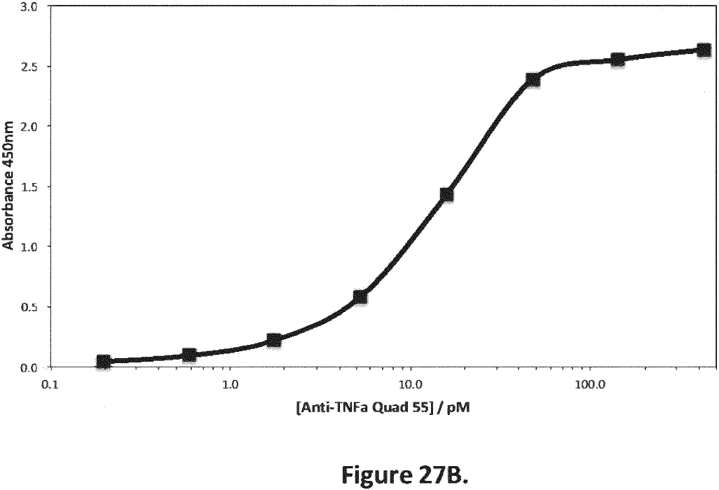

The specific scFv binding domains used in this example has specificity for anti-TNFa and anti-IL17A. The anti-TNFa scFv sequence was adapted from Humira and the anti-IL17A scFv sequence was adapted from Ixekizumab (Eli Lilly) into a bispecific Quad format (Quad 55) (Seq ID: 144). To confirm soluble expression, Quad 55 was expressed in HEK293 cells and the secreted protein was purified from culture supernatant followed by protein analysis. (FIG. 27A). Protein analysed by SDS-PAGE confirmed soluble expression of this bispecific scFv Quad format with high purity (>99%). To further anaylse whether this format was functional, ELISA binding assay was performed for the anti-TNFa scFv arm (FIG. 6B). A dose-dependent increase in binding of Quad 55 to recombinant human TNFa could be seen confirming Quad 55 was assembled correctly. Furthermore, Q55 appears to have a similar half-maximal binding in the low pM range to that of Quad 51 detailed in Example 10.

### Example 13: Expression and Binding Analysis of Bispecific Tetravalent scFv X dAb Quad

In this example, two different binding domain formats with specificity for two different target proteins were linked to the N- and C-terminus of p53 tetramerisation domain respectively, as exemplified schematically in FIG. 22-G.

The first binding domain was an anti-TNFa scFv as detailed in Example 12, which was linked to the p53 tetramerisation domain via the N-terminus. The second binding domain was an anti-IL17A dAb sequenced, which was linked to the p53 tetramerisation domain via the C-terminus as detailed in Example 9 (Quad 56) (Seq ID: 145).

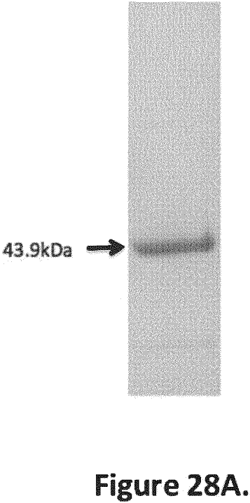

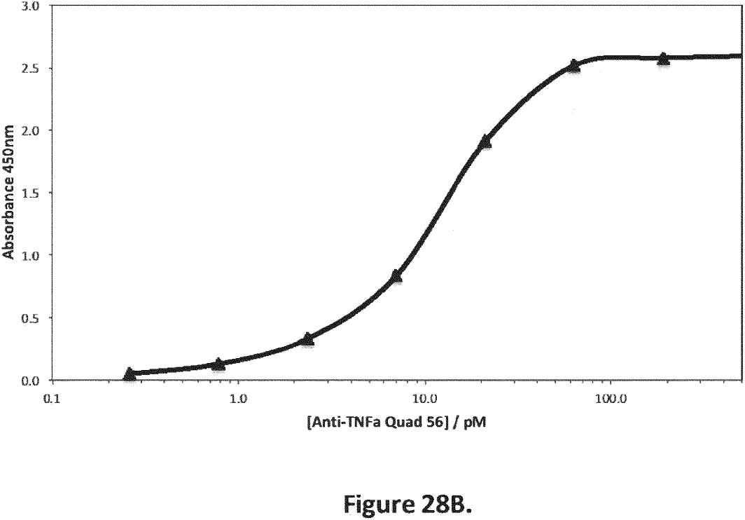

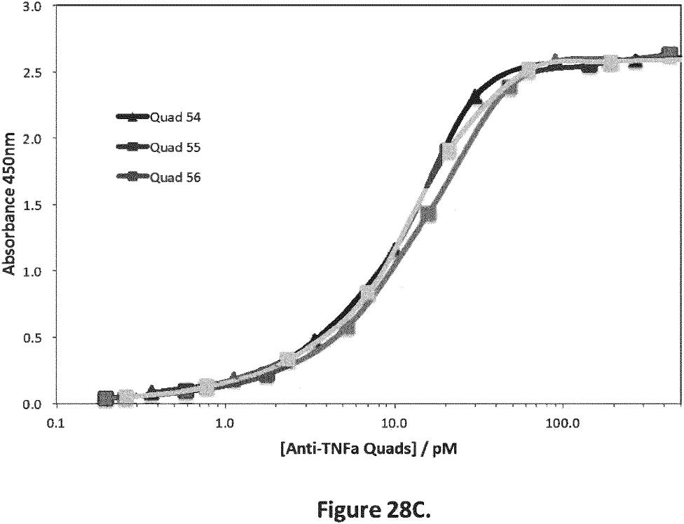

Soluble expression of this bispecific tetravalent scFv x dAb Quad format was confirmed by analysing the purified protein on SDS-PAGE gel (FIG. 28A). In additional ELISA binding assay was performed for the anti-TNFa binding arm (FIG. 28B), where a dose-depended increase in binding was observed. This confirmed correct expression and assembly of this bispecific scFv × dAb Quad format.

In the examples above (Examples 10, 12 & 13) different bispecific Quad formats (Quads 54-56) with specificity for anti-TNFa x anti-IL17A were produced and analysed for expression and functionality by ELISA binding assay for the anti-TNFa binding arm. From the data presented thus far, it is clear that the p53 tetramerisation domain is highly versatile and amenable to fusion with different binding domains at either or both N- and C-terminus. To compare the functional binding strengths of Quads 54-56, the ELISA binding assay data for the anti-TNFa binding arm of the different bispecific Quads were plotted side-by-side (FIG. 28C). The dose-response curves for all three bispecific Quad formats appear to be highly similar indicating that the binding domain format (scFv or dAb) does not affect binding. This further highlights the versatility and utility of the p53 tetramerisation domain to generate highly pure multivalent soluble proteins with high binding strength.

### Example 14: Expression and Binding Analysis of Tetravalent Monospecific Ig scFv Quad V1

In this example, an scFv binding domain was linked to the lower hinge/CH2 domain of IgG1 Fc without the core and upper hinge region to generate a scFv monomeric Ig Fc (scFv-mFc), ie wherein the Fc does not pair with another Fc when a multimer is formed using the polypeptide monomer. Typically a CXXC motif comprising cysteine residues present in the core hinge region is responsible for forming inter-chain disulfide bonds. Thus, by excluding the core hinge region, the Fc region is restrained from forming a tightly packed homodimer structure typically found in native IgG antibodies. The lower hinge/CH2 domain was kept intact to allow proper interaction with Fcγ-receptors required for effector function.

The scFv-mFc was engineered into a Quad format by linking it to the p53 tetramerisation domain via the N-terminus to generate a tetravalent monospecific Ig scFv Quad termed version 1 as schematically represented in FIG. 22-I(ie, embodiment I shown in FIG. 22D).

In this example the upper hinge was also removed, but in other examples the upper hinge region can be optionally retained completely or only partially kept intact to generate dAb monomeric Ig Quads (exemplified schematically in FIGS. 22-H, L, W, X, Y, Z, AA, AB & AC), scFv monomeric Ig Quads (exemplified schematically in FIGS. 22-I, M, X, & AA) and Fab monomeric Ig Quads (exemplified schematically in FIGS. 22-J, K, N & O) with monospecific, bispecific, trispecific or tetraspecific specificity. Although in this specific example, human IgG1 was used as an example, the Fc region could be any of the other IgG isotypes including IgG2, IgG3, gG4 or derivative thereof. The amino acid sequences encoding human IgG hinge regions are shown in Table 12.

The scFv binding domain used in this specific example was that of an anti-CD20 described in Example 10. The anti-CD20 scFv was linked to the lower hinge/CH2 domainof the Fc via a peptide (G4S)3 linker. The p53 tetramerisation domain was linked directly to the C-terminus of the CH3 domain without any peptide linkers (Quad 64) (Seq ID: 148). An optional linker could be included at this junction between the CH3 domain and the multimerisation domain to provide flexibility.

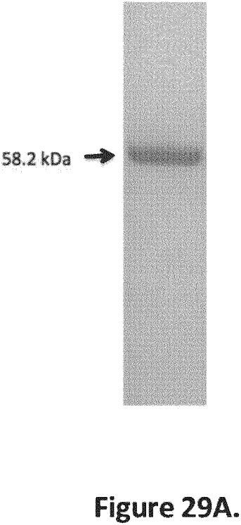

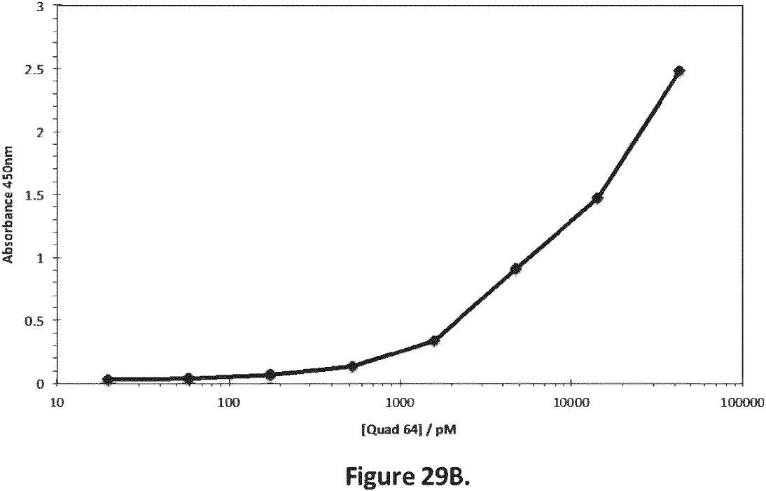

Following Quad 64 expression and purification, protein was quantified and analysed by SDS-PAGE (FIG. 29A). Protein quantification using Nanospec confirmed high protein yield after HisTrap HP column purification with protein yield equivalent to 130 mg/L. In addition, a single protein band at the expected size (58.2 kDa) as seen on the SDS-PAGE gel confirmed expression of a highly pure (>99%) tetravalent monomeric Ig scFv Quad protein. To analyse whether the expressed Ig Quad protein was assembled correctly, ELISA binding assay was performed (FIG. 29B). Quad 64 was found to retain binding to recombinant human CD20 in a dose-dependent manner confirming that it was assembled correctly and functionally active.

### Example 15: Expression and Binding Analysis of Tetravalent Monospecific Ig scFv Quad V2

In this example, a different version of the tetravalent monomeric Ig scFv Quad described in Example 14 was constructed where the anti-CD20 scFv binding domain was linked directly to the N-terminus of the p53 tetramerisation domain. The mFc containing the lower hinge, CH2 and CH3 domains was directly linked to the C-terminus of the p53 tetramerisation domain. This version of tetravalent monomeric Ig scFv Quad is termed version 2 and is schematically represented in FIG. 22-M(ie, embodiment M shown in FIG. 22F).

Although in the specific example described below, the upper hinge was not included, in other examples the upper hinge region can be optionally retained completely or only partially kept intact.

In other examples, the version 2 configuration could contain dAb binding domains as exemplified schematically in FIGS. 1-L, X, Z & AA, producing Quad molecules with monospecific, bispecific or trispecific specificity. In another example, the binding domain could be a Fab as exemplified schematically in FIGS. 22-N & O. In yet another example, version 2 could be made without mFc to give tetravalent Fabs either as monospecific (exemplified schematically in FIGS. 22Q & R-) or bispecific (exemplified schematically in FIG. 22-S).

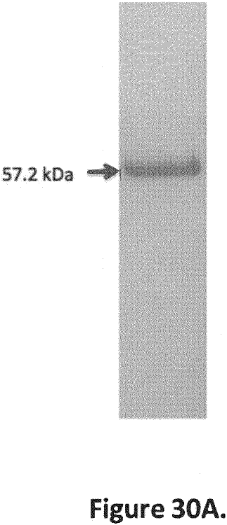

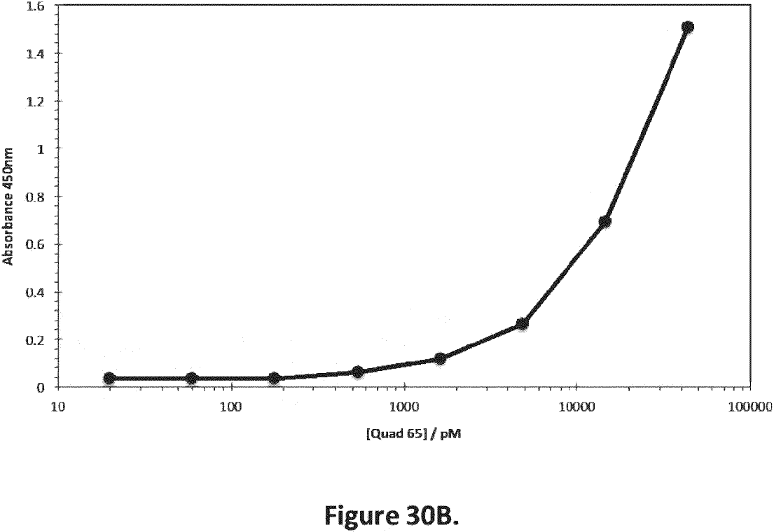

The expression construct for the tetravalent monomeric Ig scFv Quad version 2 specific for CD20 (Quad 65) (Seq ID: 149) was expressed in HEK293 cells and the soluble secreted protein was analysed by SDS-PAGE. Protein quantification using Nanospec confirmed high protein yield after HisTrap HP column purification with protein yield equivalent to 160 mg/L. In addition, a single protein band at the expected size (57.2 kDa) as seen on the SDS-PAGE gel confirmed expression with high purity (>99%) (FIG. 30A). The integrity of the expressed protein was analysed by ELISA binding assay. Recombinant human CD20 protein was used in a direct binding ELISA using serially diluted Quad 65 protein. Quad 65 was found to bind CD20 in a dose-dependent manner (FIG. 30B) confirming it assembled correctly and functionally active.

The data outlined above and in Example 14, represents the first examples of soluble and functional expression of tetravalent monomeric Ig molecules. Such multivalent monomeric Fc formats would have several advantages over scaffold and antibody fragment based molecules lacking Fc. Firstly, the presence of an Fc region in a monomeric Quad format would allow neonatal Fc receptor (FcRn) binding providing an extended half-life of these molecules in-vivo. Secondly, the presence of an Fc region would have the ability to bind multiple Fc receptors to induce effector functions such as antibody-dependent cell-mediated cytotoxicity (ADCC) and complement-dependent cytotoxicity (CDC) as reported for monovalent monomeric Ig Fc molecules (Ying et al. 2017; Ying et al. 2012). Thirdly, the proof-of-concept data outlined in this example suggest that any given monoclonal IgG antibody could be rapidly formatted into a multivalent monomeric Ig Quad format as schematically represented in FIGS. 22-J, K, N & O). With these formats retaining the native binding domain architecture of an IgG antibody, multivalent monomeric Ig Quad formats would have substantially increased binding strength for its target protein due to the increased avidity gained through multivalency. Such monomeric Ig Quad format will provide novel class of therapeutic molecules with enhanced target protein neutralization potential (Boruah et al. 2013; Shen et al. 2019) as well as having enhanced efficiency to cross-link cell surface receptor to mediate apoptosis (Li et al. 2018) and induce signaling through receptor super-clustering with enhanced agonistic potentials (Mayes et al. 2018).

### Example 16 Expression of Octavalent Bispecific Quads

In a specific example of an octavalent bispecific Quad, dAb binding domains for PD-L1 and 4-1BB were used for the dual targeting of an immune checkpoint inhibitor and an immune co-stimulatory molecule for the treatment of cancers. In other examples, dAb binding domains could have specificity for any immune checkpoint inhibitor and any co-stimulatory molecule or a mixture thereof to provide either a dual checkpoint inhibitor bispecific multimer (e.g. PD-L1 x CTLA-4) or a dual checkpoint inhibitor and immune co-stimulatory bispecific multimer (e.g. PD-L1 x 4-1BB) or a dual immune co-stimulatory bispecific multimer (e.g. 4-1BB x OX40).

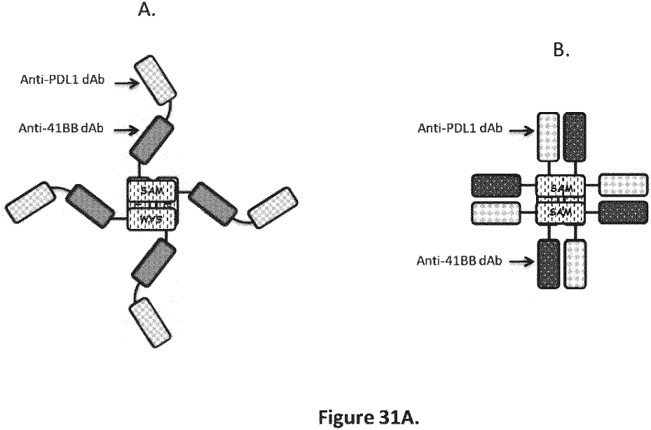

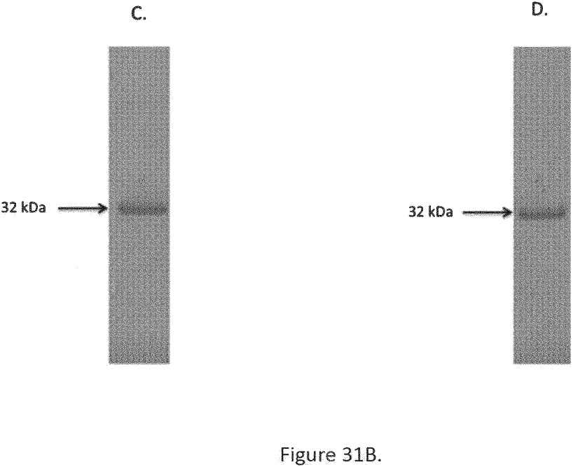

To exemplify octavalent bispecific Quads, two different versions were constructed to demonstrate soluble Quad protein expression. In one format, the PD-L1 and 4-1BB dAb binding domains were linked to a p53 multimerisation domain as a tandem dAb at either the N- or C-terminus as schematically represented in FIG. 31-A.

In a second example of an octavalent bispecific multimer, the dAb binding domains for PD-L1 and 4-1BB were linked to the p53 tetramerisation domain at opposite ends respectively as schematically represented in FIG. 31-B.

The dAb binding domain sequence for PD-L1 and 4-1BB were adapted from WO2017/123650A2. In the first version of an octavalent bispecific Quad, a tandem dAb containing anti-PD-L1 and anti-4-1BB in a N-C terminus orientation was linked to the N-terminus of p53 tetramerisation domain without any peptide linkers. This version is referred to as Quad 68 (SEQ ID NO: 190). However, in other examples, the tandem dAb can be linked to the tetramerisation domain via an optional peptide linker and as further described in Table 8-P.

In a second version of octavalent bispecific Quad multimer, the anti-PD-L1 dAb was linked to the N-terminus of p53 tetramerisation domain and the anti-4-1BB dAb was linked to the C-terminus. This version is refered to as Quad 69 (SEQ ID NO: 191). In this specific example the dAbs were linked to the tetramerisation domain without any peptide linkers. However, in other examples, dAbs can be linked to the tetramerisation domain via an optional linkers and as further described in Table 8-C.

The expression construct for Quads 68 and 69 were expressed in HEK293 and the secreted soluble proteins were purified from cultured supernatant. The purified Quad proteins were analysed by SDS-PAGE (FIGS. 31-C & D). Protein quantification of Quad 68 and Quad 69 using Nanospec confirmed high protein yield after HisTrap HP column purification with protein yield equivalent to 297 mg/L and 360 mg/L respectively. The presence of a single protein band for both Quad 68 and Quad 69 at the expected size (32 kDa) as seen on the SDS-PAGE gel confirmed expression with high purity (>99%) (FIGS. 31-C and D).

The configuration of the dAbs either in tandem (as in Quad 68) or in opposite orientation (as in Quad 69) are anticipated to engage with cancer cells via PD-L1 with higher potency and selectively than to T cells expressing 4-1BB. Therefore, T cells will preferentially be recruited to cancer cells only once the cancer cells are engaged with the Quad molecule allowing selective T cell activation with improved safety profile.

### Example 17: Expression of Tetravalent Monomeric Ig dAb Quad and Octavalent Fab-Like dAb Monomeric Ig Quad

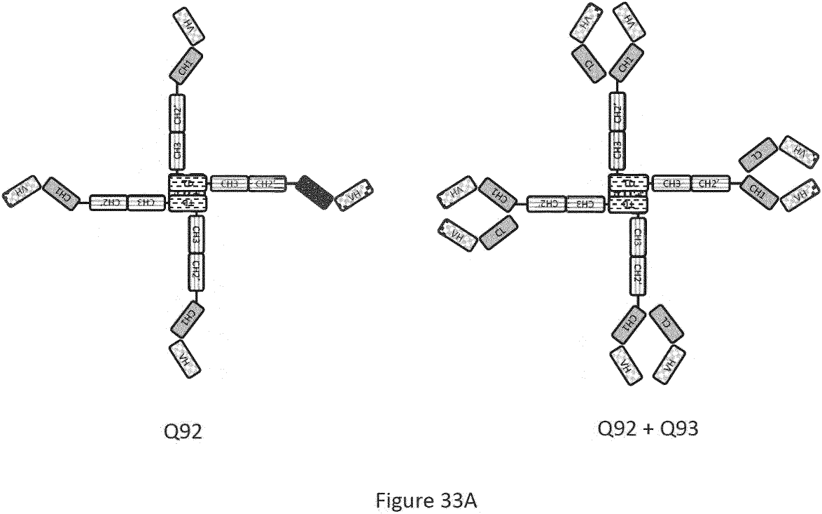

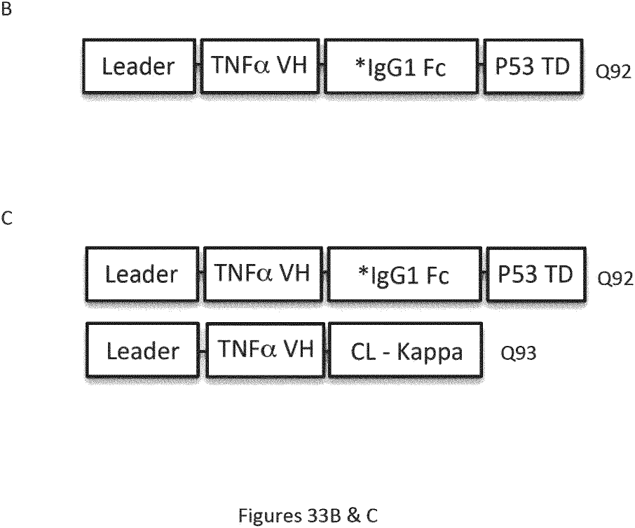

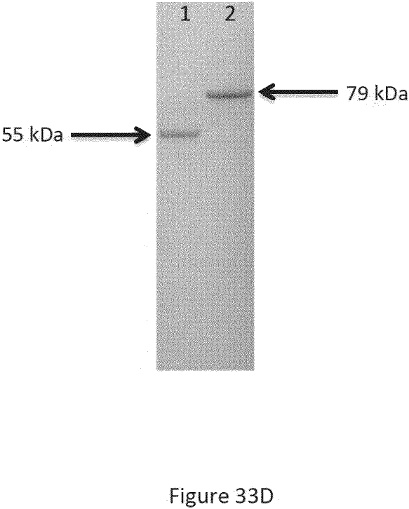

A dAb VH with specificity for TNFα as detailed in Example 9 was also used in this example to generate two new versions of monomeric Ig dAb Quads. In the first version, anti-TNFα dAb VH was linked directly to IgG CH1. The CH1 region was linked to the IgG1 Fc where the hinge region was modified so that the core hinge was removed (SEQ ID NO: 168 hinge sequence was used). The Fc region was linked to the N-terminus of the p53 TD domain yielding Quad 92. The presence of CH1 region in Q92 allowed for the generation of a second version of monomeric Ig Quad where Q92 when co-expressed with anti-TNFα dAb linked to Kappa light chain constant domain yielded an octavalent anti-TNFα Fab-like dAb monomeric Ig Quad (Q93) as schematically represented in FIG. 33A. The molecular design of Q92 and Q93 is schematically represented in FIGS. 33B&C.

Q92 alone or Q92 plus Q93 were expressed in HEK293 cells and the Quad proteins were purified directly from the culture supernatant. The purified proteins were analyzed by SDS-PAGE where pure products at the expected molecular weight (Q92 (tetravalent) - 55 kDa and Q92+Q93 (octavalent) - 79 kDa) could be seen confirming soluble expression of these Quad formats (FIG. 33D). Although in this specific example, the dAb tetravalent version retained the CH1 region, in another example the CH1 domain could be removed to generate a slightly modified version of tetravalent dAb Quad as schematically represented in FIG. 22F (embedded in Figure as L).

### Example 18: Expression of Monospecific and Bispecific Octavalent Tandem dAb Monomeric Ig Quads

In this example, two different versions of monomeric Ig Quads were generated similar to that exemplified in Example 14. However, instead of using scFv as binding domains, in this specific example antibody single domains (VH) were linked in tandem with specificity for either PDL1 alone or PDL1 plus 4-1BB. In the first of these examples, a bispecific octavalent anti-PDL1/4-1BB monomeric Ig dAb Quad (Q113) was generated by linking in tandem an anti-PDL1 dAb with an anti-4-1BB dAb separated by a flexible linker. This tandem bispecific binding module was linked to the lower hinge/CH2 region of IgG1 Fc without the core hinge region to generate a tandem dAb monomeric Ig Fc (ie, the resulting polypeptide comprised (in N- to C-terminal direction) an anti-PDL1 dAb, an anti-4-1BB dAb, lower hinge/CH2 region of IgG1 Fc without the core hinge region and IgG1 CH3) . The Fc region was linked to the N-terminus of p53-TD domain to generate a bispecific tandem dAb monomeric Ig Fc Quad as schematically represented in FIG. 22O. In this example, the binding valency of both the anti-PDL1 and anti-4-1BB dAb domains were tetravalent for their target protein. This is an example of a multivalent 4 + 4 (octavalent) bispecific antibody containing Ig Fc region. The second example was exactly the same as Q113, except the tandem dAb consisted only of anti-PDL1 dAb domains generating an octavalent anti-PDL1 monomeric Ig Quad (Q114).

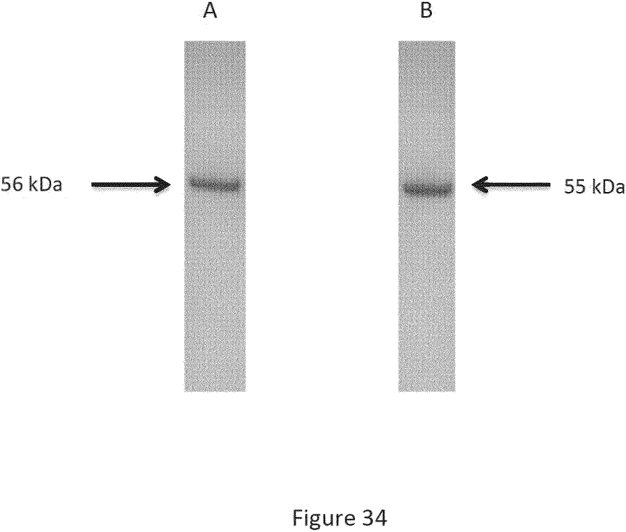

Following Quad 113 and Q114 expression in HEK293 cells and purification of proteins from culture supernatant, the recovered proteins were analysed by SDS- PAGE (FIGS. 34A & B). For both Q113 and Q114, a single pure protein band at the expected molecular weight was observed confirming expression of these monomeric Ig Quads as highly pure (>99%) soluble Quads proteins.

### Example 19: Expression of Fab Monomeric Ig Quad

In this specific example, monomeric Ig Quads were produced from IgG monoclonal antibodies where the native pairing of the variable heavy chain (VH) and variable light chain (VL) was kept intact. This was achieved by taking the Fab fragment of IgG monoclonal antibody and converting it into a monomeric Ig Quad as schematically represented in FIG. 22E (embedded in Figure as J). To exemplify such Quad format, Fab fragments from two clinically approved monoclonal antibodies were used (adalimumab (Humira™) and avelumab (Bavencio™)). The VH-CH1 domain of either Humira or Avelumab was linked to the lower hinge/CH2 region of IgG1 Fc without the core hinge region. The Fc region was linked to the N-terminus of p53-TD domain to generate the monomeric Ig Quad chain. To generate Humira and avelumab Fab monomeric Ig Quad, the native light chain VL-CL was co-expressed with the respective monomeric Ig Quad chain in HEK293 cells.

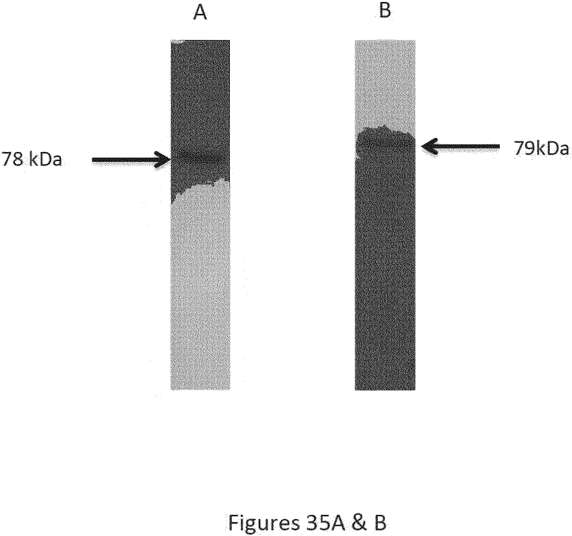

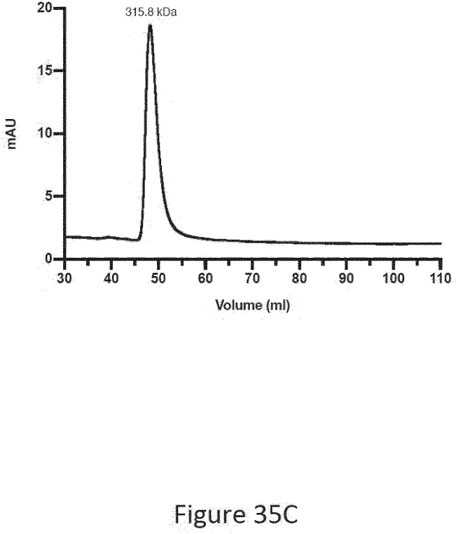

Fab monomeric Ig Quad proteins were purified from the culture supernatant and analysed initially by SDS-PAGE (FIGS. 35A & B). Detection of a single protein band at the expected molecular weight on a non-reduced denaturing SDS-PAGE gel corresponding to either Humira or avelumab Fab monomeric Ig Quad, confirmed both soluble expression and high purity of these monomeric Ig formats. The absence of any detectable free HC or free LC in these Fab monomeric Ig Quad protein preps further confirmed the high stability of these formats. To confirm the native molecular mass and oligomeric state of these Fab monomeric Ig Quad proteins, Humira Fab monomeric Ig Quad protein was analysed by size-exclusion chromatography (FIG. 35C). The size exclusion chromatography profile for Humira Fab monomeric Ig Quad protein had a clear dominant peak eluted at the expected volume consistent with the tetrameric molecular weight of 315.8 kDa. These data further confirm the high purity of these Quad proteins, which are stable and present in a homogeneous tetrameric form without the presence of any aggregates. Further examples of Fab monomeric Ig Quad could be generated from any given monoclonal antibody where the intact Fab is used to generate Quads without altering the native VH and VL pairing or it’s native antigen binding potential.

### Example 20: Expression of Extracellular Protein Domain as Monomeric Ig Quads (Q96)

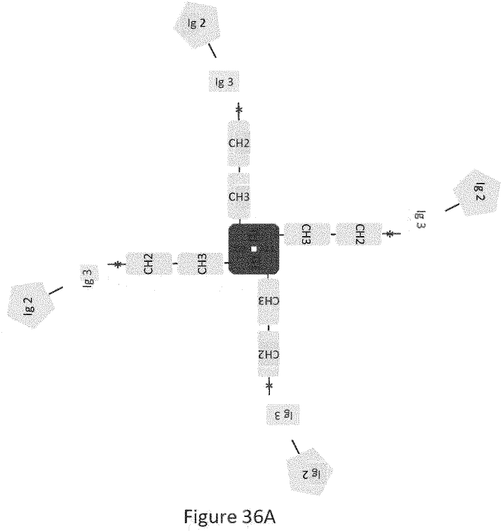

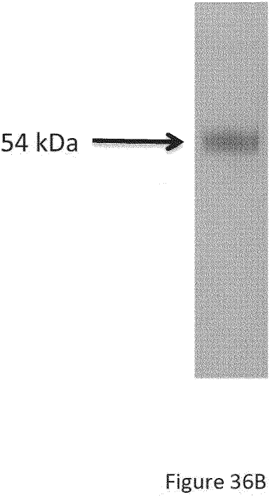

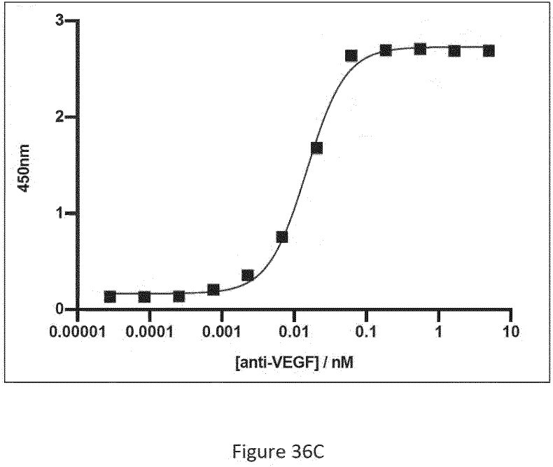

In the above examples, antibody fragments were used to generate Quads. In this specific example the versatility of p53 TD domain was demonstrated further whereby extracellular domains of cell surface receptors were multimerised into a Quad format without affecting its ability to bind its natural ligand. To exemplify this, the soluble extracellular domains used in the VEGF trap aflibercept (Eylea™) was used as an example. The Ig domain 2 from VEGFR1 and Ig domain 3 from VEGFR2 similar to Eylea was linked to monomeric Ig Quad similarly to that exemplified in Examples 17-19 and as schematically represented in FIG. 36A (Q96).

Q96 was expressed in HEK293 cells and soluble protein was purified directly from the culture supernatant. SDS-PAGE analysis confirmed expression of Q96 as a highly pure Quad with a single protein band at the expected molecular weight (FIG. 36B). The integrity of Q96 was analysed further by ELISA binding assay as described above to confirm binding to VEGF-A ligand. ELISA plates were coated with VEGF-A at 0.5 ug/ml and serially diluted (1 in 3 folds diluted starting from 5 nM) Q96 protein was added to the coated VEGF-A plate. Anti-His HRP detection antibody was used for detection of Q96 binding to VEGF-A. Q96 bound VEGF-A in a dose dependent manner confirming this format containing extracellular domains of VEGFR1 and VEGFR2 assembled correctly and it retained it’s native ligand binding potential (FIG. 36C).

### Example 21: Expression, Binding and Functional Potency of Non-Ig Tetravalent and Non-Ig Octavalent Anti-TNFα dAb Quads

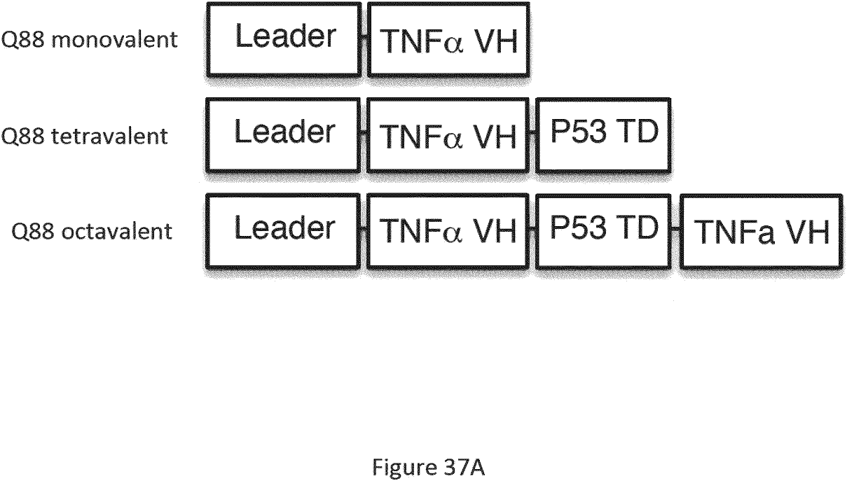

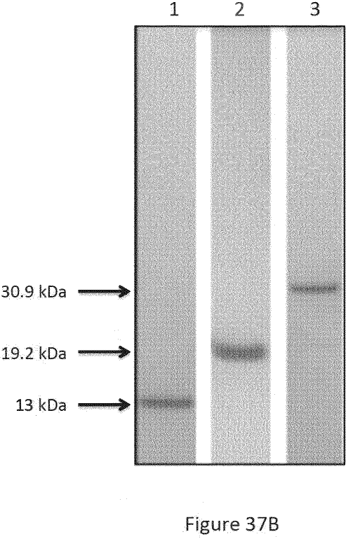

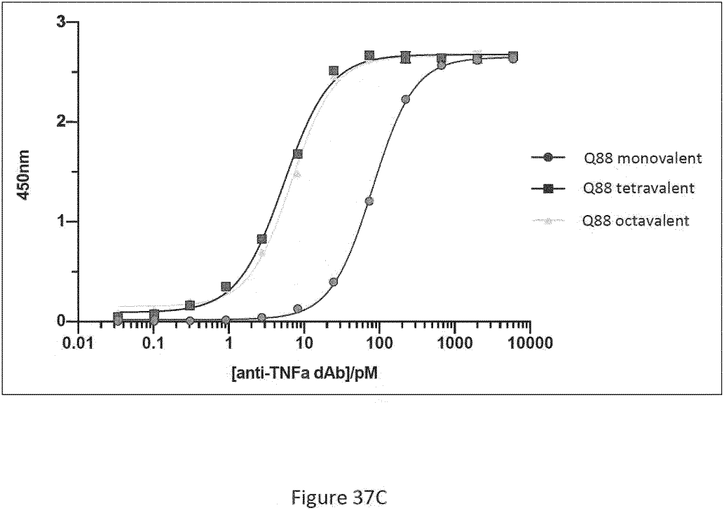

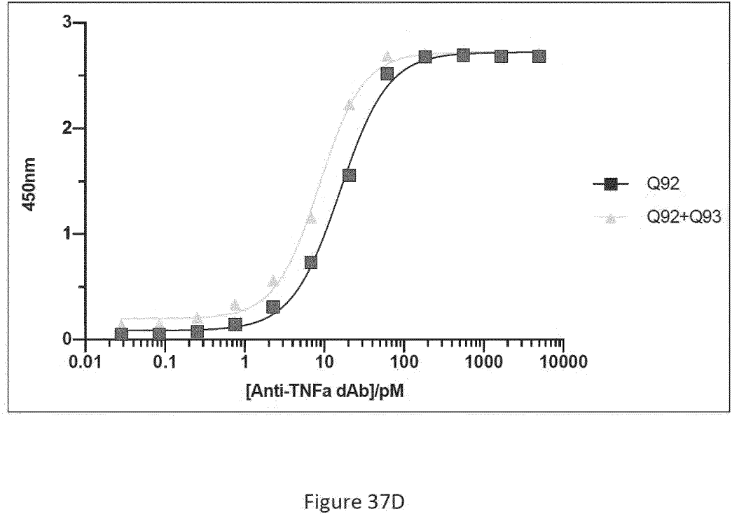

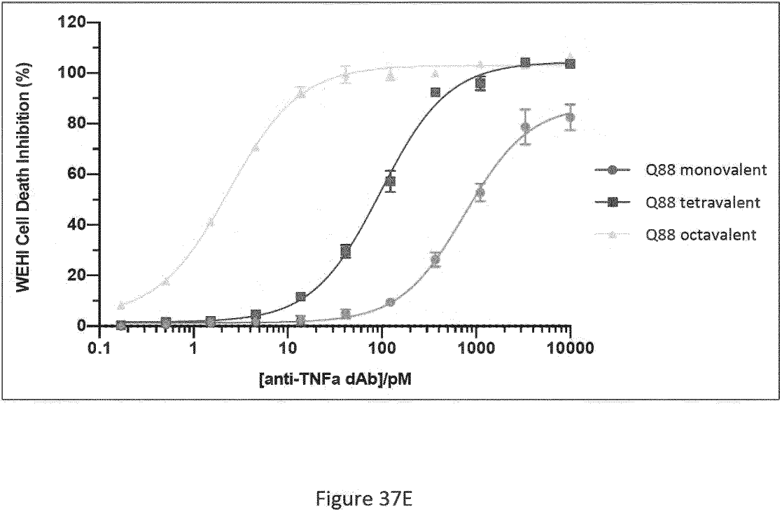

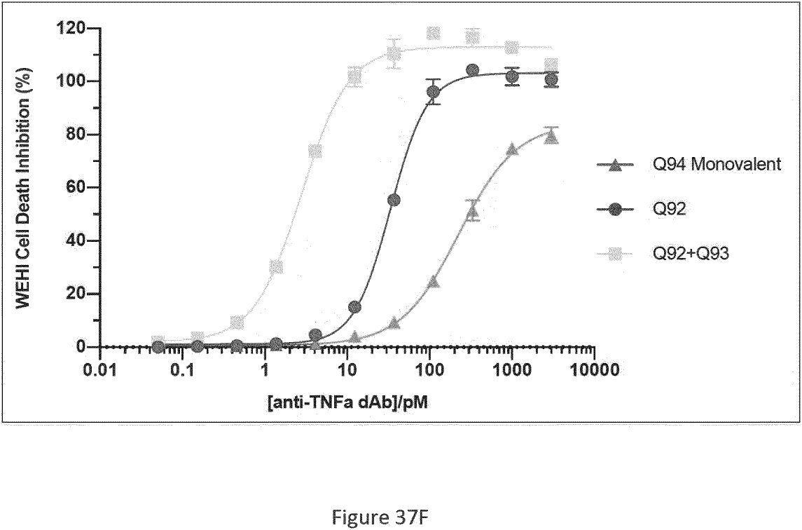

Similar to Example 8, antibody single domains were used to generate monospecific tetravalent and octavalent Quads without IgG Fc (referred to herein by shorhand “non-Ig”). Anti-TNFα dAb VH was linked directly to p53 TD at either the N-terminus to generate tetravalent Quad (Q88 tetravalent) or at both N- and C-terminus to generate octavalent anti-TNFα dAb Quad (Q88 octavalent) as schematically represented in FIG. 22A (embedded in figure as A and B respectively). The original anti-TNFα dAb without the TD domain was also produced for use as a monovalent control (Q88 monovalent). The molecular designs are schematically shown in FIG. 37A, which also highlights the modular design of these Quads.

Following expression in HEK293 cells and Quad protein purification directly from culture supernatant, initial protein analysis was carried out by SDS-PAGE. The multivalent anti-TNFα dAb Quads expressed as highly pure proteins as judged by a single band on the SDS-PAGE gels corresponding to the expected molecular weight (FIG. 37B). To further analyse these multivalent Quads and the effect avidity has on TNFα binding and TNFα neutralization, ELISA binding assay and WEHI cell-based bioassay were performed as described in Examples 9 and 10. Increase in TNFα binding strength can be seen for both tetravalent and octavalent anti-TNFα dAb Quads compared to the anti-TNFα monovalent control in ELISA binding assay (FIG. 37C). Surprisingly, the TNFα binding potential between the octavalent and the tetravalent versions could not be resolved in the ELISA binding assays. Similarly, surprisingly the binding strength for the tetravalent and octavalent anti-TNFα dAb monomeric Ig Quad versions detailed in Example 17 could not be resolved and only a small increase in TNFα binding strength was observed in ELISA binding assay (FIG. 37D). This may be because the binding strength is greatly enhanced and the tetramethylbenzidine-based colorimetric signal is rapidly saturated by these multivalent Quads, and thus the dynamic detection range for this ELISA binding assay is not adequate to differentiate the enhanced binding strength beyond a certain point.

Increase in TNFα binding domain valency was further investigated in WEHI bioassay where the potency of the anti-TNFα Quad molecules to neutralize TNFα-mediated cytotoxicity of WEHI cells were compared. WEHI bioassay was performed as described in Example 10 using both non-Ig and Ig-like anti-TNFα dAb Quads (FIGS. 37E & F). Unlike the ELISA binding assay, in this cell-based bioassay, surprisingly a substantial increase in potency can be seen with increasing anti-TNFα binding domains including major difference in potencies between the tetravalent and octavalent versions. The EC₅₀ values and the fold enhancement of potency of these molecules are summarized in Table 15.

### Example 22: Expression and Functional Potency of Non-Ig Dodeca and Hexadeca Valent Anti-TNFα dAb Quads

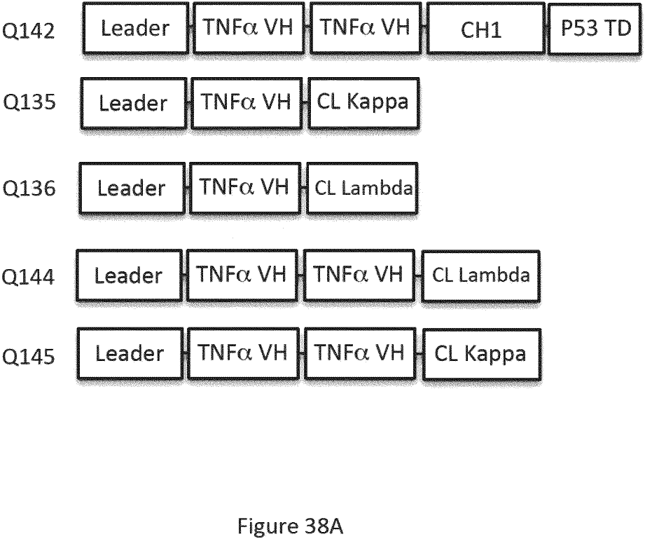

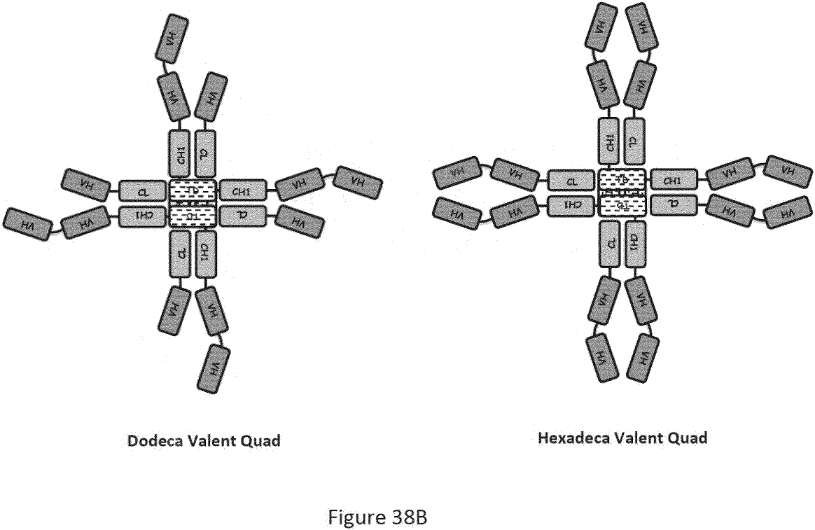

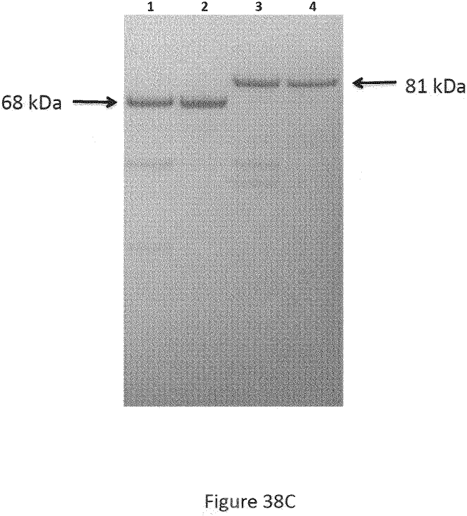

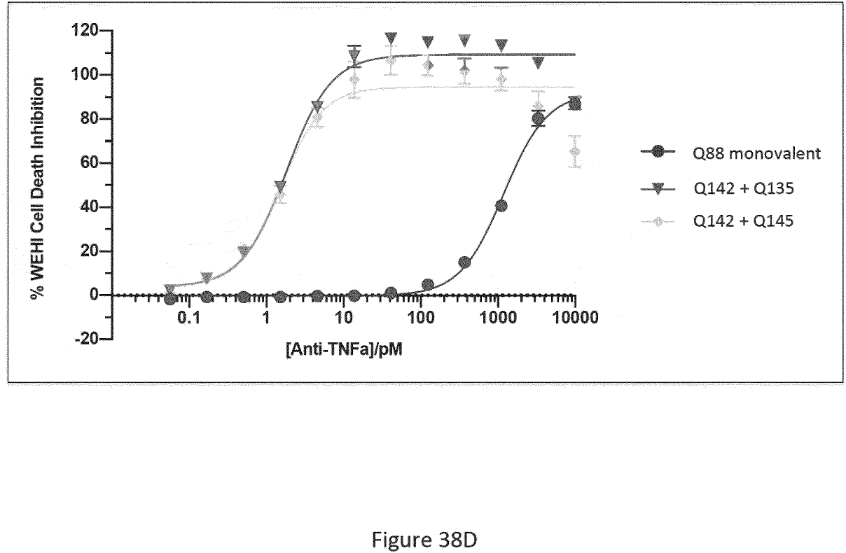

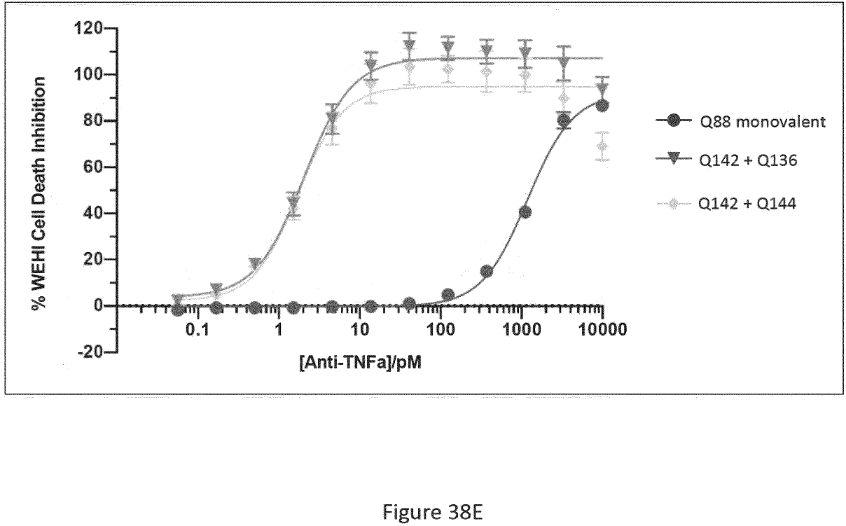

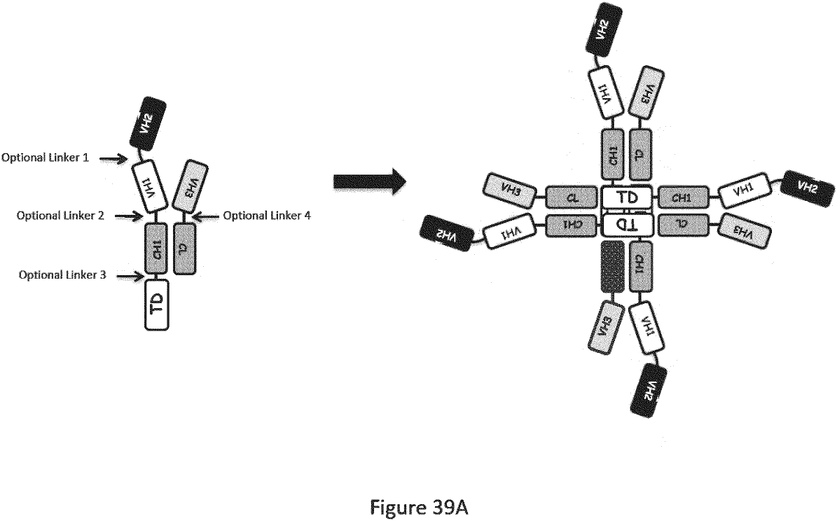

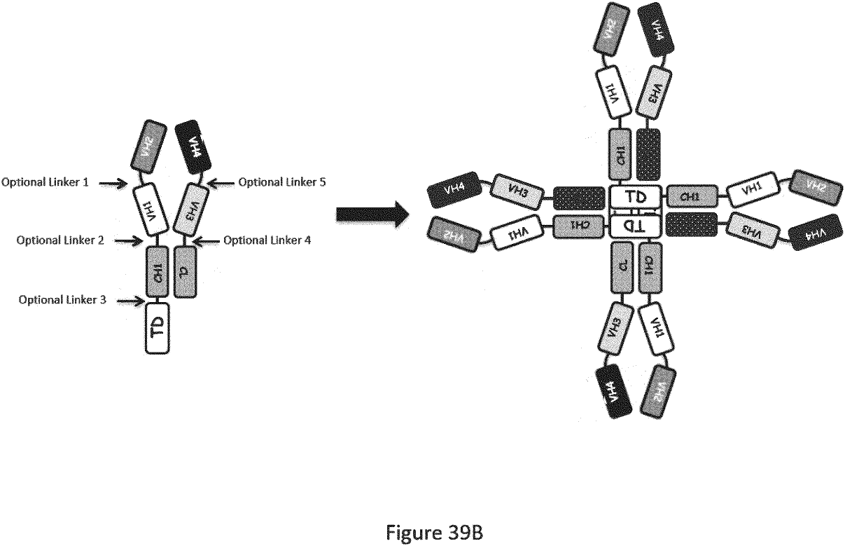

To extend the concept of multivalency further and beyond tetra- and octa-valency, two further formats of anti-TNFα dAb Quads were generated with either 12 (AKA dodeca, 12-valent or 12-mer herein) or 16 (hexadeca, 16-valent or 16-mer herein) anti-TNFα dAb binding domains in a non-Ig format. The modular design and structural arrangements of these Quads can be seen in the schematic illustration in FIGS. 38A&B.

In the dodeca version, the first chain contained tandem anti-TNFα dAbs linked to IgG CH1, which in turn is linked to the TD domain (Q142). The second chain contained a single anti-TNFα dAb linked to either kappa (Q135) or lambda (Q136) light chain constant region. Dodeca-valent Quads were generated by co-expressing the two chains in HEK293 cells where heterodimerisation of the two chains occurred through interaction between CH1 and C-kappa or C-Lambda constant region. The TD domain allowed tetramerisation of these two assembled chains into a tetramer.

For the hexadeca-valent anti-TNFα dAb Quad, the first chain was exactly the same as the dodeca valent format, however, the second chain contained a tandem anti-TNFα dAb linked to either kappa (Q145) or lambda (Q144) light chain constant region. Co-expression of these two chains allowed generation of hexadeca-valent anti-TNFα Quads.

The dodeca- and hexadeca anti-TNFα dAb Quads were expressed in HEK293 cells and the Quad proteins were purified directly from culture supernatant. The purified proteins were analysed by SDS-PAGE (FIG. 38C). The presence of a predominant protein band at the expected molecular weight confirmed these multivalent anti-TNFα dAb Quads can be expressed as highly pure soluble Quad proteins. Furthermore, it confirmed that these multivalent Quads can be generated using either kappa or lambda light chain constant region.

WEHI bioassay was performed using the purified dodeca- and hexadeca anti-TNFα dAb Quads and the TNFα neutralization potency was compared to the monovalent anti-TNFα dAb control (FIGS. 38D&E). The increase anti-TNFα dAb binding domain valency in the dodeca-and hexadeca-valent Quad formats substantially increased the TNFα neutralization potency. The EC₅₀ of these Quads are summarized in Table 16.

Thus, it has been demonstrated that constructs of the invention can surprisingly achieve highly significant increases in antigen binding potency (762-fold in Table 16, for example) and advantageously this can be achieved using different types of binding site (eg, dAb or Fab-like), with or without antibody Fc presence, with possibility of repurposing clinically approved antibodies to retain their tested binding sites and at very high purity levels (almost 100% purity).

### Example 23: Expression Non-Ig Tetravalent Fab Quad

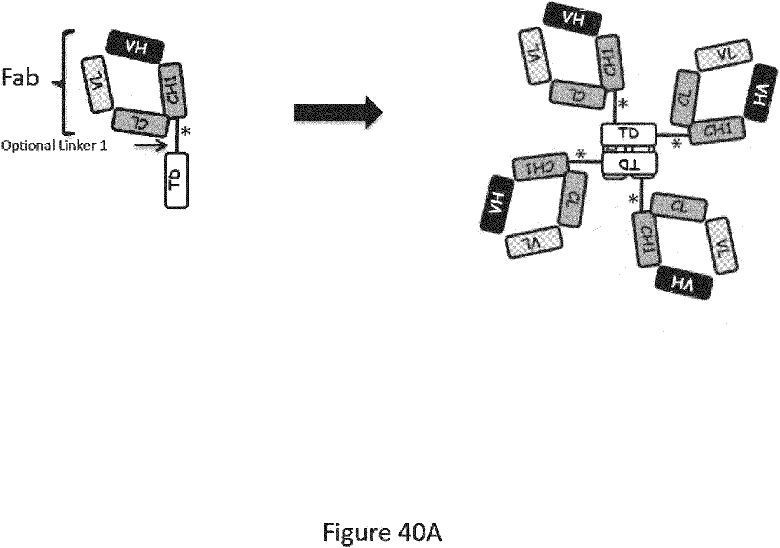

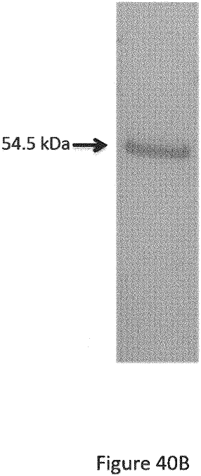

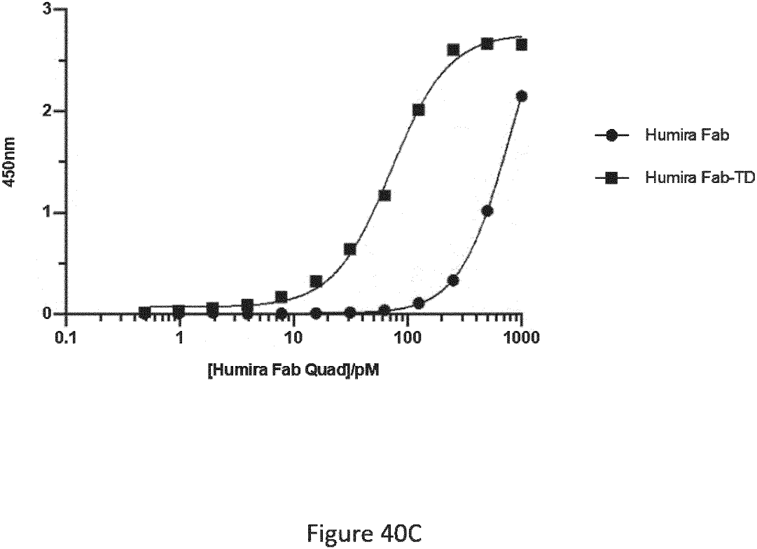

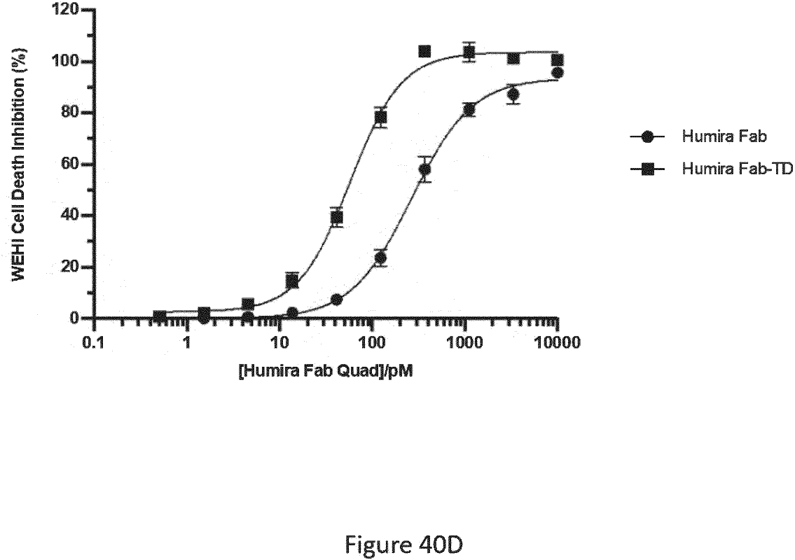

In example 19 production of tetravalent Humira Fab monomeric Ig Quad was exemplified. In this specific example, Humira Fab with intact light chain (LC) and heavy chain (HC) but without Fc region (non-Ig version) was generated. The p53 TD domain was linked at the C-terminus of Humira HC CH1 domain where the hinge region was modified to be devoid of core hinge (ie, upper hinge sequence without core or lower hinge sequence; SEQ ID NO: 183 was used). Co-expression of this modified HC with the native Humira LC, allowed generation of tetravalent Humira Fab-TD with significantly reduced molecular size compared to the Humira Fab monomeric Ig-TD version. A schematic structural representation of Humira Fab-TD is shown in FIG. 40A. “Non-Ig” multimer refers to Quad multimers where the starting monomeric building block does not contain an Fc, as suppose to an “Ig-like” multimer where the starting monomeric building block contain Fc.

The Humira Fab-TD Quad was expressed in HEK293 cells and the Quad protein was purified directly from culture supernatant. The purified protein was analysed by SDS-PAGE (FIG. 40B). The presence of a single protein band at the expected molecular weight confirmed that Humira Fab-TD could be expressed as a soluble protein with high purity (>99% pure).

To further characterize this Quad protein, TNFα binding assay using ELISA and TNFα neutralization potential using WEHI bioassay was performed. As a control Humira Fab was used as a monovalent control. From the ELISA binding assay, it can be seen Humira Fab-TD could bind TNFa with higher binding strength than the Humira Fab monovalent control (FIG. 40C). Similar in the TNFα neutralization bioassay, Humira Fab-TD was able to neutralize TNFα mediated toxicity at higher potency than Humira Fab monovalent control (FIG. 40D). These data confirm that by increasing the valency, the functional affinity is increased resulting in stronger binding and this also enhances the functional potency of Quads compared to the monovalent control.

### Example 24: Octavalent Fab as Non-Ig and Ig-Like Quads

In the examples above, Fabs from antibodies were used to generate tetravalent Quads either as Ig-like (Example 19) or non Ig-like (Example 23). Further iterations of Fab Quads can be made to generate Fabs with octavalent valences either as Ig-like or non Ig-like.

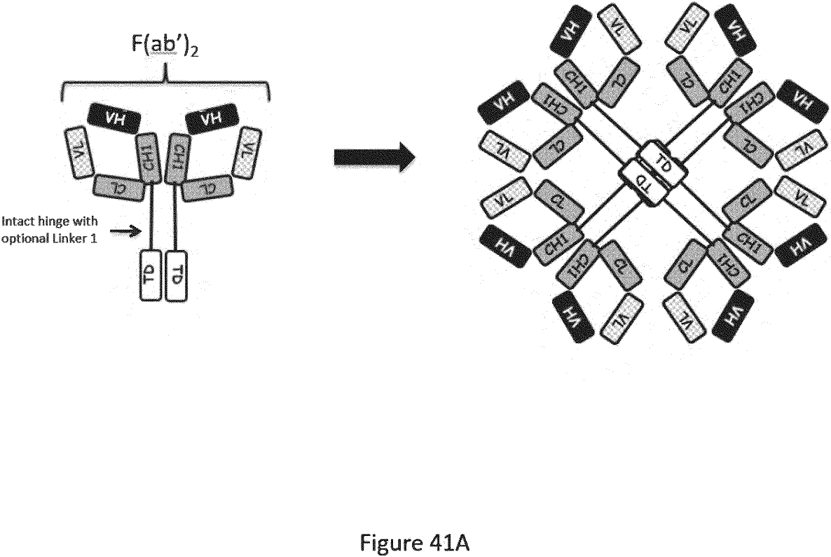

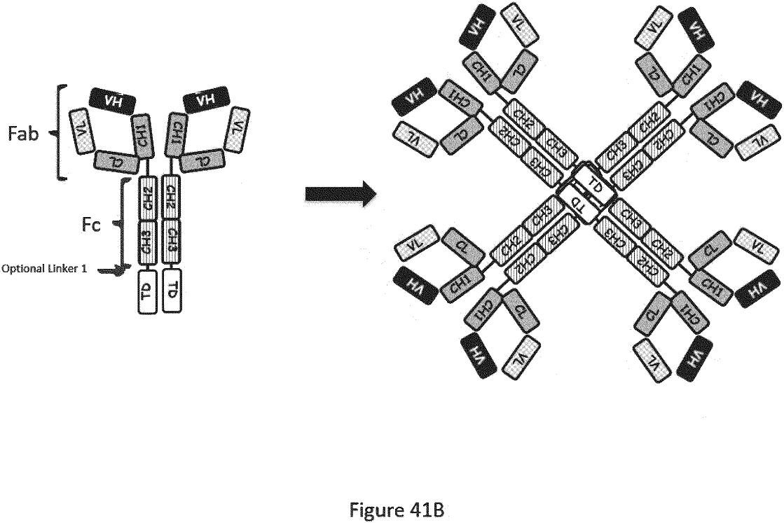

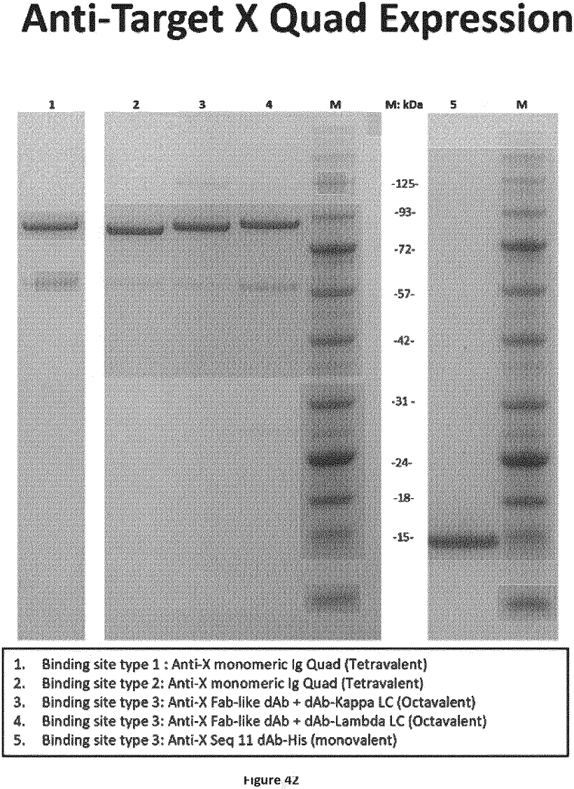

To generate octavalent non Ig-like Fab Quads, a Fab with an intact hinge region will be used where p53 TD domain is linked directly to the hinge region optionally via a flexible peptide linker. The intact core hinge region will allow homodimerization of the Fab-TD when co-expressed with its native LC to generate F(ab′)₂ and as such the monomeric building block will be bivalent. In turn, the TD domain will allow tetramerization of the F(ab′)₂ to generate an octavalent Fab Quad as schematically shown in FIG. 41A.

Similarly, to generate octavalent Ig-like Fab Quads, a TD, eg, a p53 TD domain, will be linked directly to the C-terminus of CH3 domain of an unmodified HC of a predetermined antibody via an optional peptide linker. When this is co-expressed with its native LC from the antibody, the monomeric building block will effectively resemble a fully assembled Ig antibody with p53 TD domains linked to it at the C-terminus of the HC of the assembled antibody. The TD domain in turn will allow tetramerization of the monomeric building blocks to generate an octavalent Fab Ig-like Quad as schematically shown in FIG. 41B. This embodiment is useful to tetramerize any predetermined antibody, such as any predetermined antibody disclosed herein. Thus, for example, a clinically approved antibody can be formatted according to the invention using SAM (eg, TD) mulimerisation to produce a multimer with many more than 2 binding sites for the cognate antigen. For example, the antibody can be bococizumab, alirocumab or evolocumab. In an alternative, instead of using an antibody with SAM (eg, TD), one can use an antigen-binding trap that comprises one or more antigen binding sites and an Fc. For example, one can use aflibercept-SAM as a monomer or aflibercept-TD (such as a p53 or homologue TD as disclosed herein). Thus, a tetramer of aflibercept will be formed by multimerization of the TDs.

Thus, an embodiment provides an antibody comprising a heavy chain, wherein the heavy chain comprises a SAM, eg, a TD (such as a p53 or homologue TD as disclosed herein). The TD may be at the C-terminus of the heavy chain. For example, the heavy chain comprises (in N-to C-terminal direction) a VH, an antibody CH1, a hinge, a CH2, a CH3 and a SAM (eg, a TD). In an example, the heavy chain is paired with a light chain (eg, wherein the light chain comprises (in N-to C-terminal direction) a VL and an antibody CL), wherein a VH and VL comprised by the heavy and light chain pair form an antigen binding site. In an example, the antibody is a 4-chain antibody, such as comprising first and second copies of the heavy and light chain pairs (ie, a first heavy chain paired with a first light chain, a second heavy chain paired with a second light chain, wherein each pair comprises a VH/VL antigen binding site, and wherein the heavy chains are paired together (such as via disulphide bonding in the constant region)). In an example, there is provided a tetramer of such an antibody, wherein the heavy chain of each antibody comprises a TD at its C-terminus and 4 copies of the antibody are tetramerised by the TDs. See, eg, FIG. 41B.

In an example, there is provided an antibody light chain comprising in N-to C-terminal direction) an antibody V domain (eg, a VL, such as a Vκ or a Vλ; or a VH), an antibody CL and a SAM, eg, a TD (such as a p53 or homologue TD as disclosed herein). For example, there is provided a multimer (eg, a tetramer) of such a light chain, optionally wherein the light chain (or each light chain in the multimer) is paired with a second antibody chain comprising (in N- to C-terminal direction) another V domain and a CH1, wherein the V domain and CL of the light chain are paired respectively with the other V domain and CH1.

### Example 25: Expanding Antigen Specificity Through Multimerisation

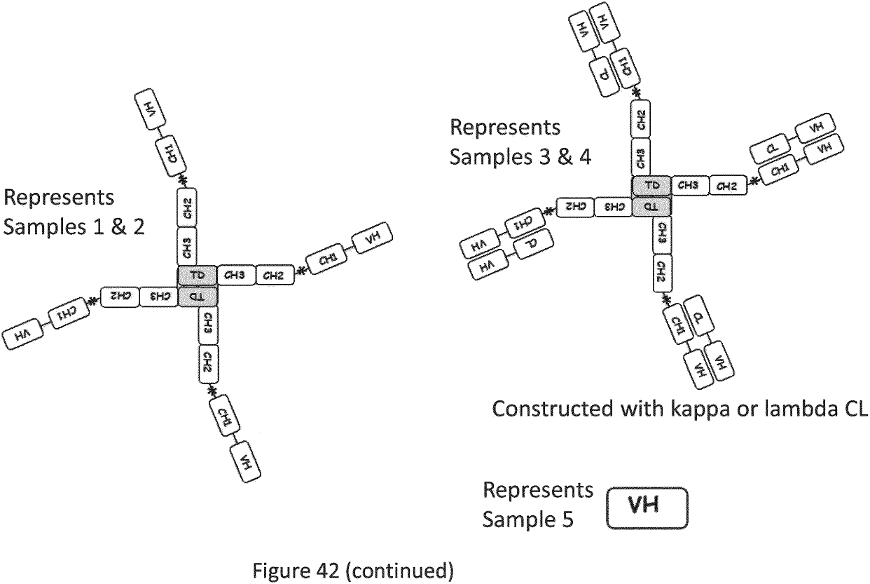

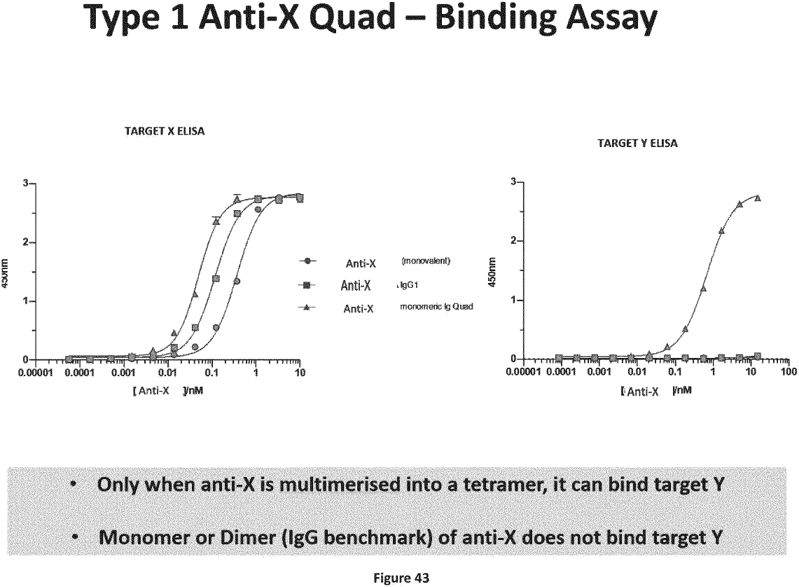

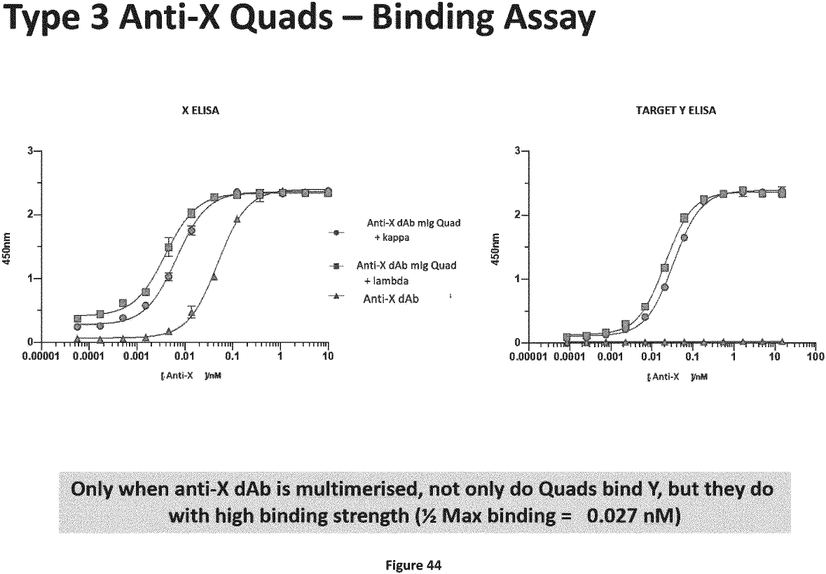

Reference is made to FIGS. 42 to 44 which demonstrate the surprising effect of multimersation to increase valency of binding sites for a cell-surface human receptor (target X). Antibodies (benchmark dAb or 4-chain antibody) having one or two binding sites for X do not bind target Y (X and Y each being cognate receptors for ligand Z). Multimers of the invention were produced each comprising 4 or 8 anti-X binding sites based on such sites of the dAb or 4-chain antibody; p53 was used as a TD. It was surprisingly found that the multimers of the invention could bind to target X and Y. X=BCMA, Y-TACI and Z=APRIL. BCMA is also known as B-cell maturation antigen (BCMA or BCM), also known as tumor necrosis factor receptor superfamily member 17 (TNFRSF17). TACI is 1so known as transmembrane activator and CAML interactor (TACI), also known as tumor necrosis factor receptor superfamily member 13B (TNFRSF13B). APRIL is also know as A proliferation-inducing ligand (APRIL), also known as tumor necrosis factor ligand superfamily member 13 (TNFSF13).

Summary:
Different anti-X antibodies currently under clinical investigation were converted into Quads (ie, multimers of the invention)
All anti-X Quads expressed well and bound target X as expected
Unexpectedly when benchmark and Quad versions of the anti-X were tested for target Y binding, both Type 1 & 3 versions bound Y when multimerised according to the invention (no such Y binding seen with benchmarks)
Multimerised Anti-X Quads are very interesting as they can bind and neutralize a second receptor (which mimics the natural ligand)
Given an antibody can bind a specific epitope, multimers of the invention could potentially be used to mimic ligands that bind multiple receptors or ligands due to enhanced binding strength A further application for multimers of the invention therefore could be in virus neutralization such as HIV or COVID 19 where virus evolves through mutations - normal bivalent antibody will have reduced binding towards the virus as it evolves and may not be picked up in screens using different virus isolates; see Example 37: Anti-SARS-CoV-2 Quads to Combat Viral Mutation where multimerization enables an anti-spike multimer to surprisingly bind to different mutated forms of SARS-CoV-2 spike. This will be useful for binding to first and second different versions of the SARS-CoV-2 virus, useful for providing resistance to evolving mutation of the virus.
Multimerisation technology of the invention could be used to identify and isolate virus neutralizing antibodies from surviving patients (ie, patients partially or completely resistance to a virus or who show reduced or no symptoms thereof).
Multimers of the invention could be used to develop antibodies with broader/stronger neutralization potential for viruses than a normal IgG antibody

### Example 26: Expanding Medical Utility of Binding Sites Through Multimerization

This example demonstrates how advantageously multimerization of the invention can repurpose a binding site which otherwise would not be useful or much less useful for medical use (eg, for treatment or prophylaxis of a disease or condition mediated by or associated with an antigen to which the binding site binds).

A multimer was produced by multimerising copies of a polypeptide. The polypeptide had the sequence of CR3022 VH Fab-TD shown in Table 23, and comprised (in N- to C-terminal direction) a single copy of the VH domain of CR3022, and a human p53TD. Each polypeptide was paired with a light chain, wherein the light chain comprised (in N- to C-terminal direction) a single copy of the VL domain of CR3022 and a human Ck domain.

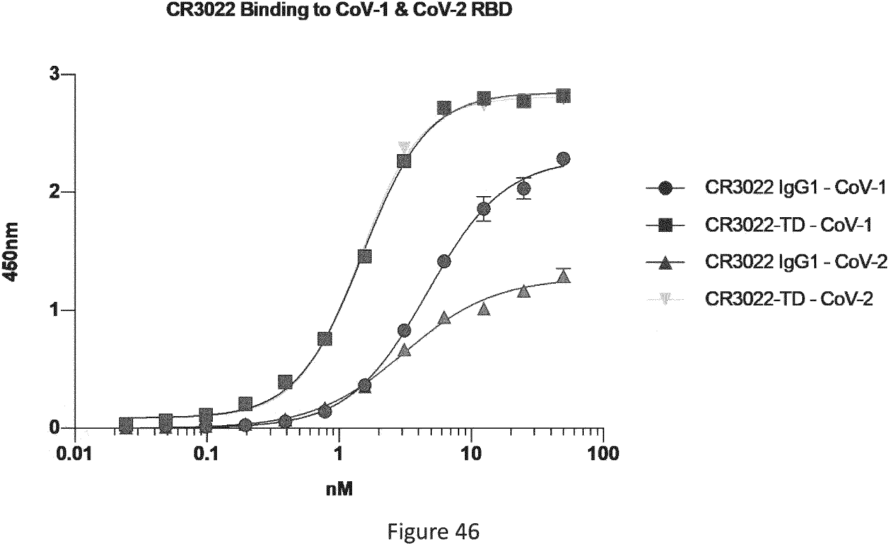

ELISA was carried out using the example assay method given above. Briefly, for FIG. 46, High binding ELISA plates were coated with either 100 ng/well of SARS CoV-1 RDB or SARS-CoV-2 RBD protein and incubated at 4° C.entigrade overnight. After blocking with 1% BSA for 1 hour at room temperature, plates were washed 3 times with PBS Tween and 100 ul/well serially diluted CR3022 IgG1 or CR3022-TD Quad was added and incubated at room temperature for a further 1 hour. The plates were washed again 3 times. HRP Protein L was added to detect CR3022 antibodies bound to RBD protein.

CR3022 is a derivative of a human antibody isolated from a patient who recovered from SARS-CoV-1 infection. This antibody is known to bind the receptor binding domain (RBD) of CoV-1 strongly. It also cross-reacts with the RBD of CoV-2 strain but with significantly lower binding affinity as reflected in the above data. However the multimer version of CR3022 according to the invention massively improves the cross-reactive binding to RBD of CoV-2 and well as improving binding to the RBD domain CoV-1 strain (FIG. 46). This makes the mulimter version of the CR3022 binding site a favorable candidate for developing as a novel SARS-CoV-2 neutralizing biologic compared to Ig antibodies. The mulimter version of the CR3022 binding site comprised 4 copies of the VH/VL antigen binding site of CR3022, as opposed to the 2 binding sites on the CR3022 Ig.

### Example 27: Expanding Assay Utility of Binding Sites Through Multimerization

This example demonstrates how advantageously multimerization of the invention can repurpose a binding site which otherwise would not be useful or much less useful for assay use (eg, detecting a pathogen or antigen that mediates, causes or is adversely associated with a disease or condition in a subject). Through multimerization of the invention, very high-order multimers (eg, containing 8-24 copies of a binding site) can easily be achieved in a stable multimer that can be readily expressed, such as in eukaryotic expression systems and host cells (as demonstrated in the exemplification above). The high-order multimers usefully can repurpose binding sites that individually have relatively low binding strength for an antigen, wherein in the multimers an avidity effect is produced rendering the combined binding strength of copies of the binding site well suited to very sensitive assay detection of low levels of antigens in samples. Usefully, for example, we demonstrate this even for very diluted samples where the antigen is at very low concentration. This is advantageous, for example where the antigen is an antigen of a pathogen (eg, a virus, bacterium or fungus that causes disease, such as in humans, animals or plants); or where the antigen is comprised by antibodies produced by a human or animal subject in response to immunisation, such as in response to a pathogen or a human protein in the subject.

A multimer was produced by multimerising copies of a polypeptide. The polypeptide had the sequence of ACE2(18-615) Ig-TD shown in Table 24, and comprised (in N- to C-terminal direction) a single copy of the ACE2 amino acids 18-615, a human hinge region, a human CH2, a human CH3 and a human p53TD.

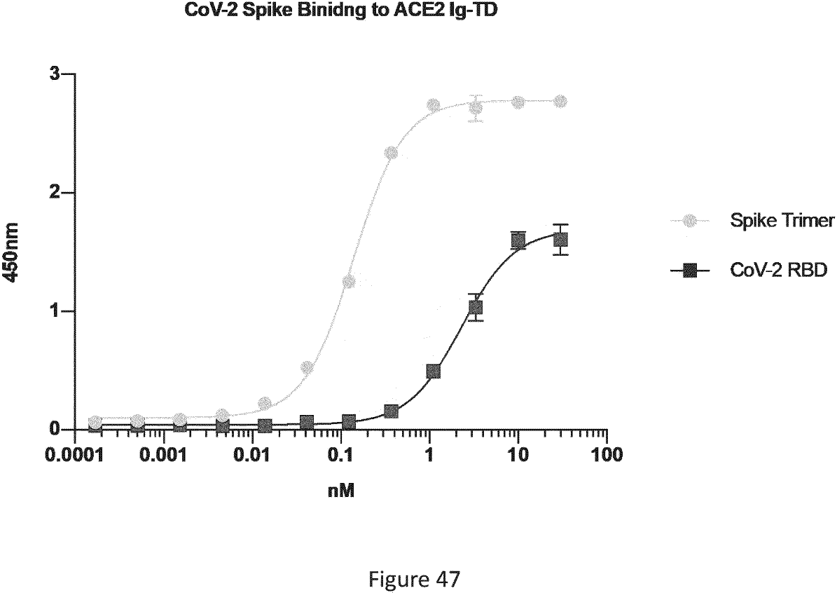

ELISA was carried out using the example assay method given above. Briefly, for FIG. 47, High binding ELISA plate was coated with 100 ng/well of ACE2 Ig-TD Quad protein and incubated at 4° C.entigrade overnight. After blocking with 1% BSA for 1 hour at room temperature, plates were washed 3 times with PBS + 0.1% Tween and 100 ul/well serially diluted SARS-CoV-2 Spike Trimer or SARS-CoV-2 RBD was added and incubated at room temperature for a further 1 hour. The plates were washed again 3 times and anti-His HRP conjugated antibody was used to detect either spike trimer or CoV-2 RBD bound to ACE2 Ig-TD.

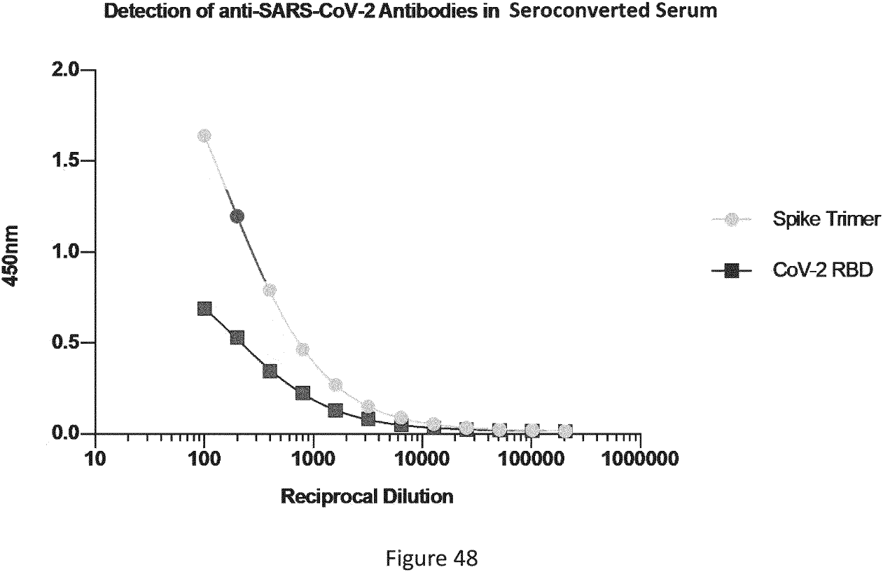

ELISA was carried out using the example assay method given above. Briefly, for FIG. 48, High binding ELISA plate was coated with 100 ng/well with ACE2 Ig-TD Quad protein and incubated at 4 oC overnight. After blocking with 1% BSA for 1 hour at room temperature, plate was washed 3 times with PBS + 0.1% Tween and 65 ul/well SARS-CoV-2 spike trimer or SARS-CoV-2 RBD at 5 nM fixed concentration was added to each well and incubated at room temperature for 1 hour. After another 3 washes using PBS + 0.1 % Tween, 65 ul of serially diluted serum starting from 1 in 100-fold dilution was added and plate incubated at room temperature for 1 hour. The plate was washed again 3 times. HRP Protein L was added to detect SARS-CoV-2 specific seroconverted human antibodies in serum.

Spike glycoprotein is naturally assembled into a trimer. It can be produced recombinantly as a trimer or only the RBD domain can be produced as a monomer for use in in vitro assays. The spike protein binds ACE2 receptor as the initial step in the infection process. Thus, ACE2 can be potentially used as a decoy receptor to neutralize coronavirus. In this experiment, a tetravalent ACE2 Ig-TD multimer Quad (ie, an example of a multimer comprising more than 2 binding sites; this example had 4 ACE2 extracellular proteins) was generated and used to capture the two recombinant forms of the spike protein for comparison. Strikingly, ACE2 Ig-TD Quad binds the trimeric form, which is more analogous to the native form with significantly enhanced binding strength than the RBD domain alone suggesting ACE2 Ig-TD is a good candidate for developing as a super neutralizer of SARS-CoV-2. See FIG. 47.

As shown in the previous FIG. 47, ACE2 Ig-TD can bind spike trimer with higher binding affinity than the RBD monomer. The captured recombinant spike proteins were used here to capture seroconverted human antibodies in serum (i.e. The fluid and solute component of blood, which does not play a role in clotting, as the skilled addressee knows). The ACE2 Ig-TD captured trimer can detect seroconverted SARS-CoV-2 antibodies in serum with higher sensitivity than ACE2 Ig-TD captured RBD domain monomer (FIG. 48). It is therefore anticipated that ACE2 Ig-TD can not only be used as a decoy to potently neutralize SARS-CoV-2 for therapeutic or prophylactic medical use, but it can alternatively be used in sensitive serological assays for detecting SARS-CoV-2 specific seroconverted antibodies. Positive detection could be made in the range from 1000 to 10000-fold dilution, thereby showing the very high sensitivity of an ELISA assay using a multimer of the invention.

### Example 28: Analysis of CR3022 and ACE2 Based Quads for Enhanced Binding and Neutralization Potency

### Methods

### Expression Vectors and Plasmid DNA Preparation

All DNA fragments were synthesized by Twist Bioscience (California) and cloned into the expression vector. Lyophilised plasmid DNA synthesized by Twist Bioscience, were resuspended with MQ water to a concentration of 50 ng/µl. Competent E. coli DH5α cells were transformed with 50 ng of DNA using a conventional heat shock method. Transformed cells were plated on LB agar plates containing 100 µg/mL ampicillin and grown overnight at 37° C. Individual colonies were picked and grown in LB broth overnight at 37° C., 220 rpm. Plasmid DNA were purified from the cells using Qiagen plasmid extraction kits, according to the manufacturers instructions (Qiagen).

### Expression of Quad Proteins in Expi293F Cells

Expi293F™ cells (Thermo Fisher Scientific) were cultured in Expi293™ Expression Medium (Thermo Fisher Scientific) according to the manufacturer’s recommendations. The only exception was that 5% CO₂ was added directly to the flasks when the cells were split and non-vented caps were used.

Two methods involving different transfection reagents were utilised for protein expression. The methods for 30 ml cultures are described below and the protocol was adapted to either scale up or down according to the experimental requirements.

For PEI transfections the cells were counted one day prior to transfection using a NC-3000™ (ChemoMetec) and were diluted to 1.5 × 10⁶ cells/ml using Expi293™ Expression Medium. The cells were cultured in 5% CO₂ at 37° C., 125 rpm overnight. The following day the cells were counted, spun down for 5 minutes at 1000 rpm and resuspended at 2 × 10⁶ cells/ml in 30 ml of fresh media. 33 ug of plasmid DNA was added to 900 ul media and 90 ul of PEI Max (Polysciences Inc.) was added to 900 ul media. The DNA and transfection reagent samples were mixed and incubated at room temperature for 15 minutes. The DNA/transfection reagent mixture was added to the cells, which were cultured as before and incubated for a further 72 hrs.

For transfections with Expifectamine™ 293 Reagent (Thermo Fisher Scientific) the cells were also diluted to 1.5 × 10⁶ cells/ml in Expi293™ Expression Medium one day prior to transfection. On the day of transfection the cells were centrifuged and resuspended at 2.5 × 10⁶ cells/ml in 30 ml of fresh media. Two tubes containing 1.5 ml of Gibco™ Opti-MEM™ (Thermo Fisher Scientific) were prepared. 30 ug of plasmid DNA was added to one tube and 80 ul of Expifectamine was added to the other. The solutions were mixed and incubated at room temperature for 30 minutes. The DNA-transfection reagent complex was added to the cells, which were cultured in 5% CO₂ at 37° C., 125 rpm. Following 16-18 hrs incubation, transfection enhancers 1 and 2 were added to the cells according to the manufacturers protocol. The cells were incubated for a further 96 hours.

### Purification of His-Tagged Proteins

The cells were harvested by centrifugation for 10 minutes at 4000 rpm. The ~30 ml supernatant was filtered through a 0.22 µm filter and diluted to 50 ml with binding buffer (50 mM HEPES, pH 7,4, 250 mM NaCl, 20 mM imidazole) containing Complete™ EDTA-free protease inhibitors (Roche) to facilitate binding to the column. A 1 ml HisTrap™ HP column (GE Healthcare) was connected to an AKTA Start (GE Healthcare) and pre-equilibrated with binding buffer. The protein-containing media was loaded onto the column using a flow rate of 1 ml/min. The column was washed with >10 CV of binding buffer before the protein was eluted using a 20-300 mM imidazole gradient over 12 ml. 0.5 ml fractions were collected and analysed by SDS-PAGE. Protein containing fractions were pooled and concentrated using Amicon® Ultra centrifugal filter units (Millipore).

### Sds-Page

Purified proteins were analysed by separating out on SDS-PAGE under denaturing condition. Typically, 1-2 µg of purified protein were loaded per lane on SDS-PAGE gel. The gels were run in Tris-Glycine buffer containing 0.1% SDS. A constant voltage of 150 volts was used and the gels were run for ~70 mins until the dye front has migrated fully.

SDS-PAGE (15% Bis-Tris) gels were prepared using the following resolving and stacking gels.

Resolving Gel:
5 ml 30% Bis-Acrylamide
2.6 ml 1.5 M Tris (pH 8.8)
50 µl 20% SDS
100 µl 10% APS
10 µl TEMED
2.2 ml MQ Water

Stacking Gel:
0.75 ml 30% Bis-Acrylamide
1.25 ml 1.5 M Tris (pH 8.8)
25 µl 20% SDS
50 µl 10% APS
5 µl TEMED
2.9 ml MQ Water

### General Binding ELISA Assay

The potential of the purified Quad proteins to bind its target protein was confirmed by indirect binding ELISA. Briefly, high binding 96 well plates (Corning) were used for coating recombinant target protein (1-2.5 ug/ml diluted in PBS or as indicated), which were typically stored at 4° C. overnight. Plates are then washed 3 times with 200 ul wash buffer (PBS + 0.1% Tween) and blocked using 200 ul blocking buffer (PBS + 1% BSA) for 1 hour at room temperature. Purified protein samples are typically serially diluted in dilution buffer (PBS + 0.1% BSA) and 100ul/well is added. Samples are incubated at room temperature for 1 hour after which the plate is washed again 3 times using 200 ul wash buffer. Detection antibodies as indicated in the text was diluted according to the manufacturer recommendation is added and incubated at room temperature for 1 hour. The plate is washed for the final time using 3x 200 ul wash buffer and 50 ul pre-warmed detection reagent (TMB - Sigma) is added per well and the plate incubated in the dark for 10-30 mins. The reaction is stopped by adding 25 ul/well of 1 M sulfuric acid. The absorbance at 450 nm was read using a CLARIOstar microplate reader (BMG Labtech). For competitive ELISA, a fixed amount of biotinylated spike protein or CoV-2 RBD protein (0.5 nM - 7.5 nM or as indicated) is mixed with the purified protein samples and pre-incubated at room temperature for 30 minutes prior to adding to protein coated ELISA plates. Europium labeled streptavidin is used as a detection reagent (Perkin Elmer).

### Analysis of Enhanced Binding and Neutralization Potency

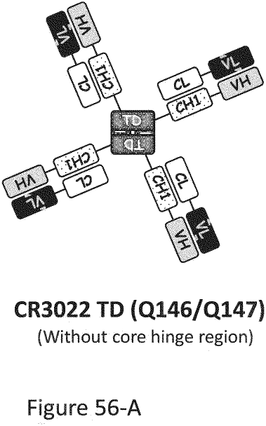

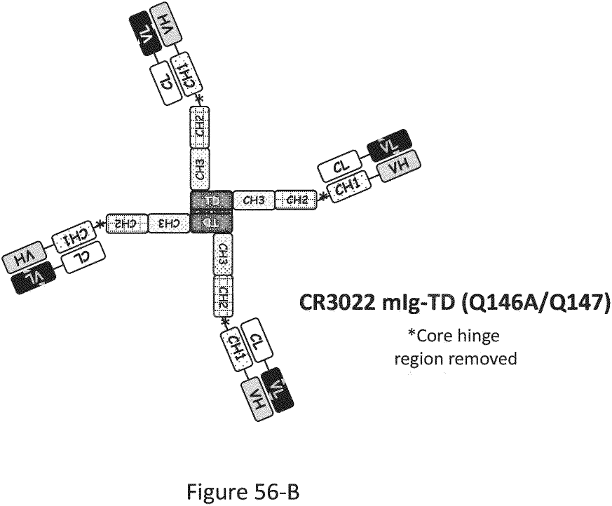

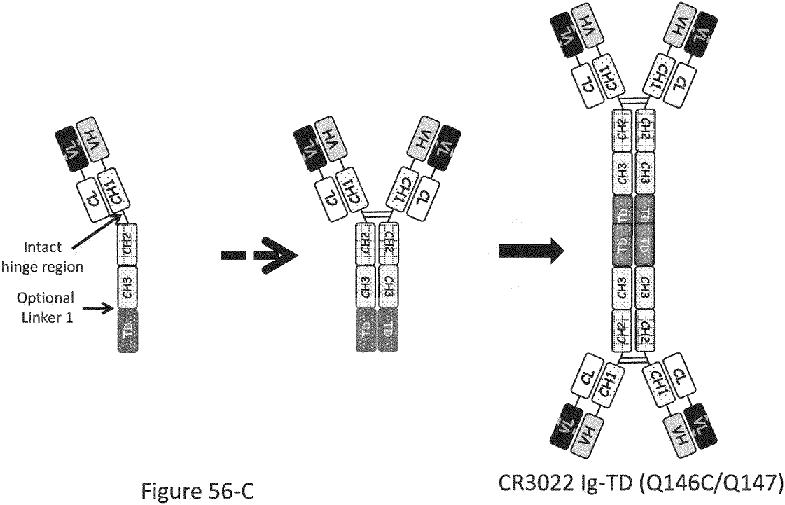

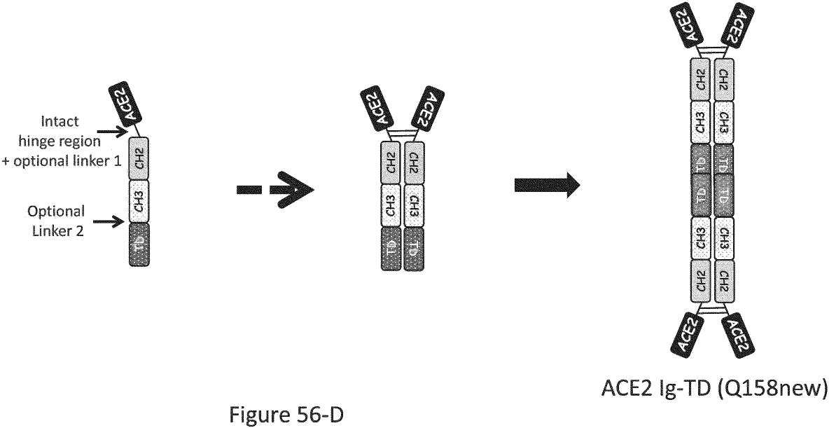

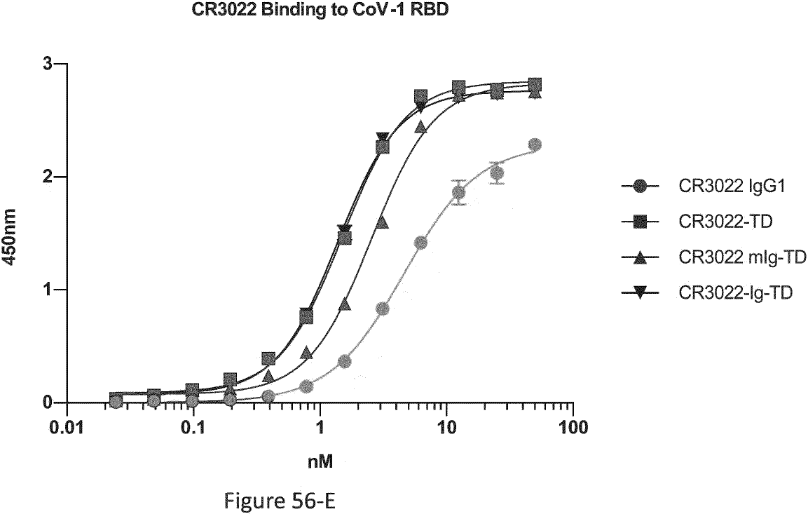

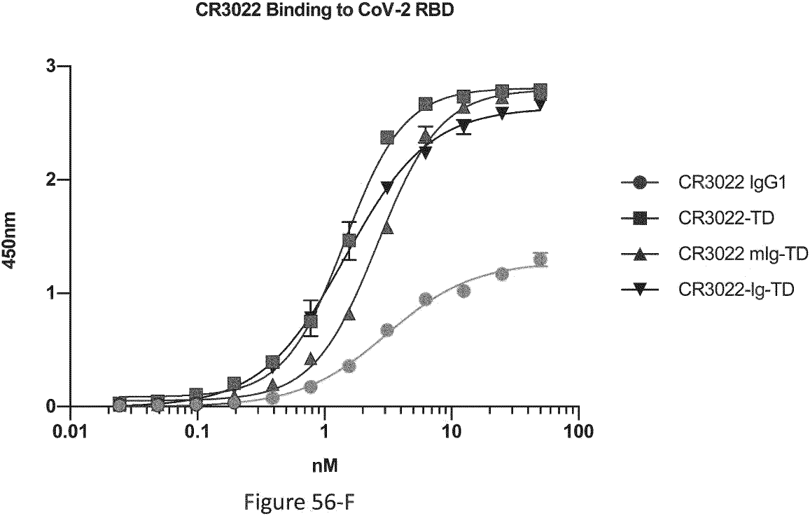

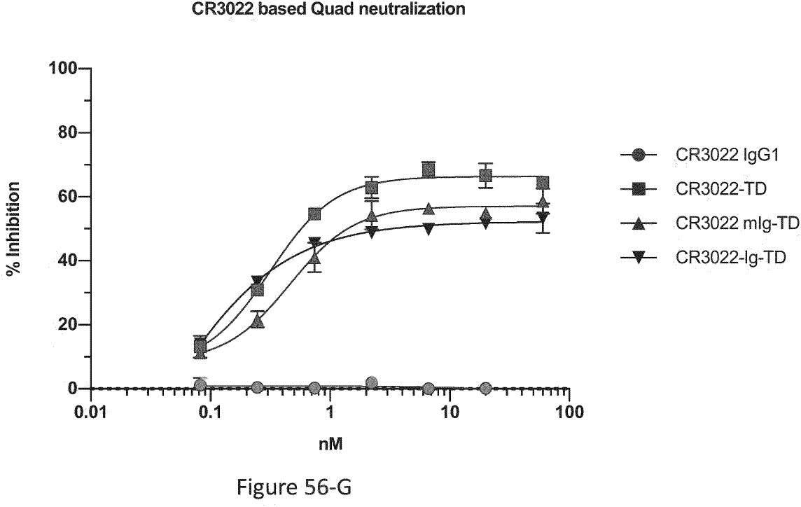

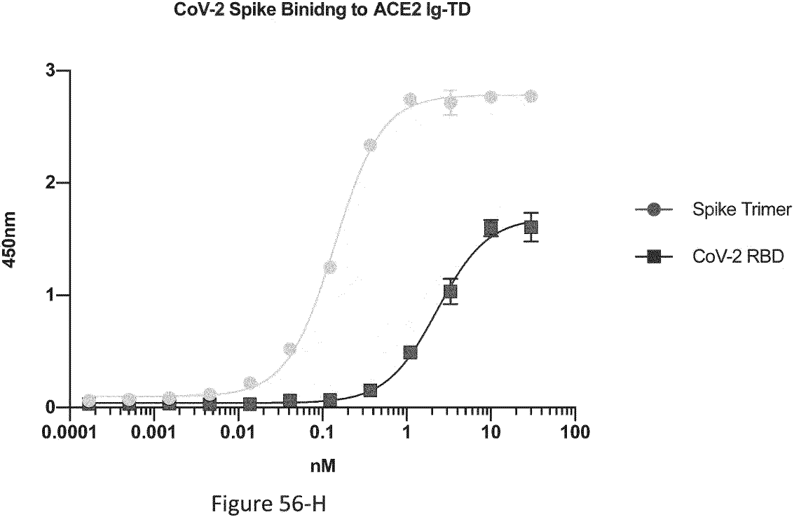

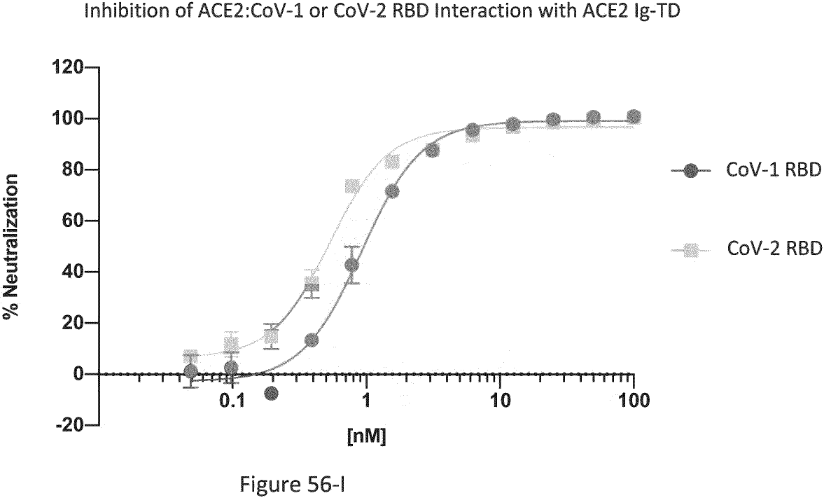

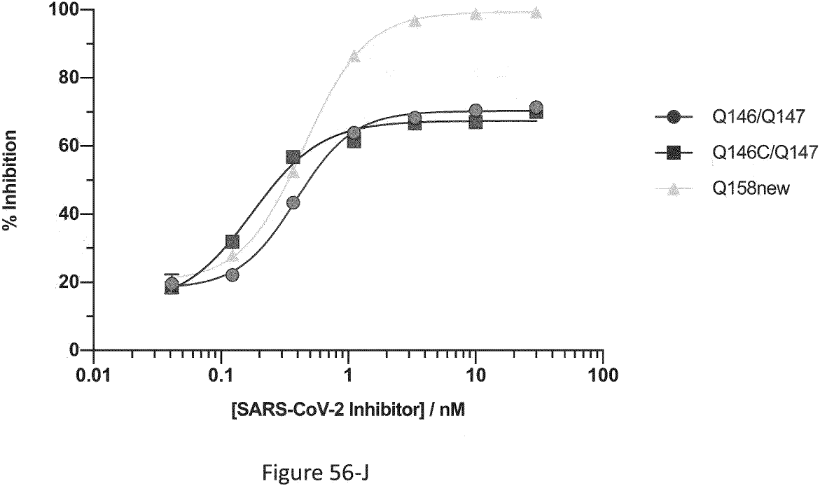

CR3022 is an antibody to SARS-CoV-1. It binds the receptor-binding domain (RBD) of CoV-1 more strongly than SARS-CoV-2 receptor binding domain (RBD). To enhance the CR3022 cross-reactive binding strength to CoV-2 RBD, the variable regions of CR3022 were used to generate different Fab Quad formats either with or without Fc region in order to increase the Fab binding domain valency from bivalent to tetravalent schematically shown in FIGS. 56A-C.

Human ACE2 is a key receptor that SARS-CoV-2 virus uses to gain entry into cells to cause infection. The receptor-binding motif of SARS-CoV-2 is the main attachment point for ACE2. Thus, to generate a decoy ACE2 Quad to neutralize virus binding ACE2 expressed on host cell surface, an ACE2 Fc fusion Quad protein was generated. The extracellular domain of ACE2 was fused to IgG Fc directly at the hinge region, which was then linked to the p53 tetramerization domain as schematically shown in FIG. 56D.

CR3022, CR3022-based Quads and ACE2 Ig-TD proteins were produced in Expi293 cells as soluble secreted proteins and their binding to CoV-1 and CoV-2 RBD was analysed by indirect ELISA. For CR3022 based Quads, high-binding ELISA plates were coated with either 100 ng of CoV-1 or 100 ng CoV-2 RBD and serially diluted antibody was added from 50 nM. Bound antibody was detected using Pro L HRP. As expected, CR3022 showed stronger binding to CoV-1 RBD than CoV-2 RDB (FIGS. 56E-F). The Quad versions of CR3022 all showed significant increase in binding to CoV-1 RBD and in particular to CoV-2 RBD binding compared to CR3022. Thus, the increased Fab binding domain valency in the Quad formats allowed enhancement in cross-reactive binding to CoV-2 RBD.

The different CR3022 Quad formats were also tested for their ability to neutralize ACE2: Spike interaction in a competitive ELISA. Recombinant ACE2-IgG (200 ng) was used to coat ELISA plates and then serially diluted antibody starting from 60 nM with a fixed amount of full-length spike trimer (7 nM) was preincubated at room temperature for 30 minutes before adding to each well. Wells with only spike protein added without any inhibiting antibody was used as 100% binding. Europium labelled streptavidin was used to detect the amount of spike protein that was bound to ACE2. CR3022 was unable to neutralize ACE2:spike interaction even at the maximum concentration used in this assay (FIG. 56G). The three different Quad versions of CR3022 were all able to inhibit the interaction between ACE2 and spike protein. The increase in the functional affinity of these Quad formats helped promote not only the cross reactive binding to CoV-2 but it also helped in promoting cross-reactive neutralization.

Similarly for the ACE2 Ig-TD tetravalent Quad, it was tested for it’s ability to bind both full-length spike protein trimer or CoV-2 RBD by indirect ELISA. ACE2 Ig-TD Quad (100 ng) was used to coat ELISA plates and serially diluted full-length spike protein or CoV-2 RBD from 100 nM was added to the coated ELISA plate. Bound spike protein or CoV-2 RBD to ACE2 was detected using anti-His HRP. ACE2 Ig-TD Quad bound both spike protein and CoV-2 RBD in a dose-dependent manner (FIG. 56H). Stronger binding of ACE2 Ig-TD to the spike trimer can be seen than the CoV-2 RBD domain. This could be due to the spike protein being a trimer whereas the RBD domain is a monomer. Nevertheless, binding of ACE Ig-TD to the spike protein and RBD confirmed ACE2 in this Quad format was fully assembled.

To examine whether ACE2 Ig-TD Quad was able to neutralize the interaction of either ACE2:CoV-1 RBD or ACE2:CoV-2 RBD, ELISA plates were coated with 200 ng of recombinant ACE2-Ig protein. Serially diluted ACE2 Ig-TD Quad from 100 nM with a fixed amount of CoV-1 or CoV-2 RBD (7 nM) was added to each well. Wells with only CoV-1 or CoV-2 RBD protein added without ACE2 Ig-TD was used as 100% binding. Bound CoV-1 RBD or CoV-2 RBD to ACE2 was detected using anti-His HRP (FIG. 56-I). ACE2 Ig-TD Quad was able to neutralize the interaction of ACE2:CoV-1 RBD and ACE2:CoV-2 RBD interaction in a dose dependent manner confirming ACE2 Ig-TD is fully functional and it has the potential to be used as a decoy molecule to prevent SARS CoV-2 infection.

To compare the neutralization potency of the CR3022 based Quads and ACE2 Ig-TD, the competition ELISA as described above was set up using 250 ng of recombinant ACE2-Ig protein to coat the ELISA plate and 7.5 nM fixed amount of CoV-2 RBD together with serially diluted CoV-2 inhibitor protein from 30 nM was added to each well. Wells with only CoV-2 RBD protein added without any inhibitor antibody was used as 100% binding. Europium labelled streptavidin was used to detect the amount of CoV-2 RBD protein that was bound to ACE2 (FIG. 56-J). The data confirmed the CR3022 based Quads and the ACE2 Ig-TD were all able to inhibit ACE2:CoV2 RBD interaction in a dose dependent manner. The ACE2 Ig-TD was able to completely block ACE2:CoV2 RBD interaction at the higher doses (>3 nM) whereas both the CR3022 Quads tested in this assay were only able to inhibit approximately 70% of the ACE2:CoV2 RBD interaction. The IC₅₀ values are summarized in Table 7.

### Example 29: Engineering SARS CoV-2 Neutralizing VHH Nanobody Into Quad Formats With Increasing Valency and Potency

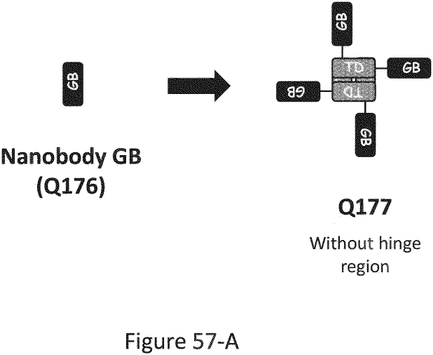

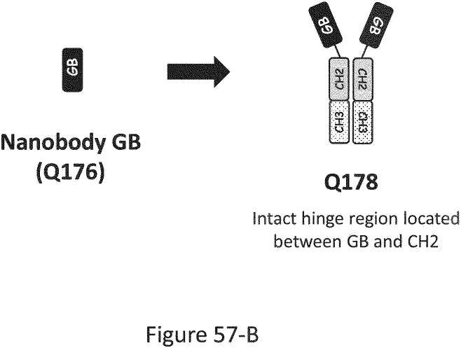

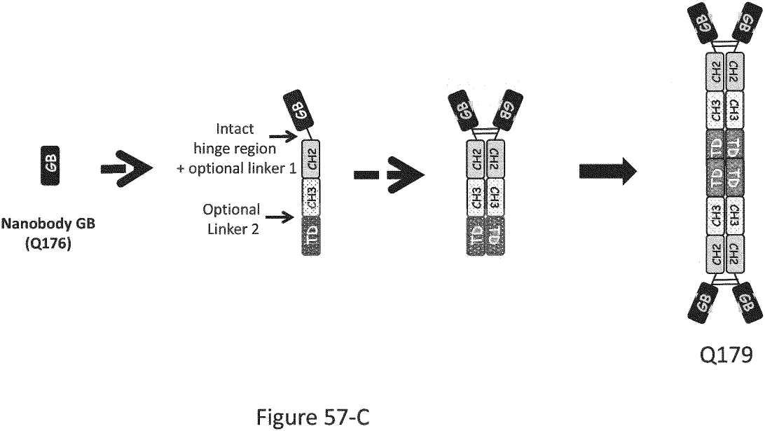

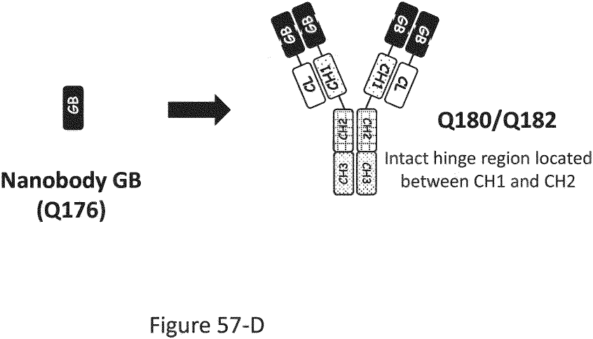

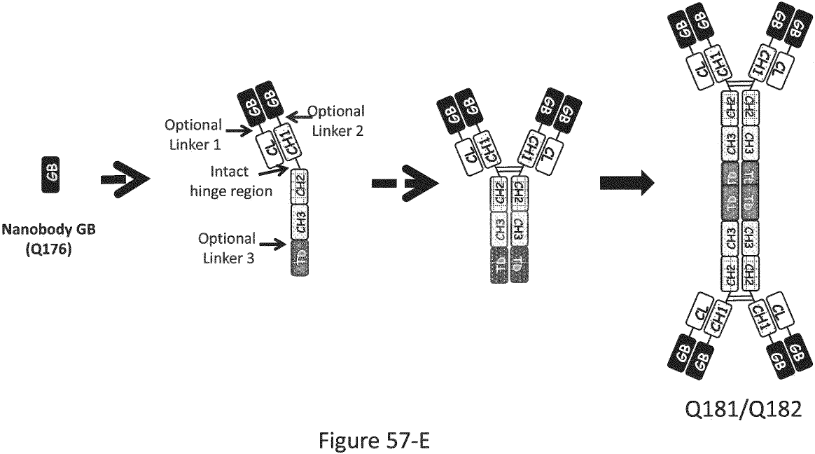

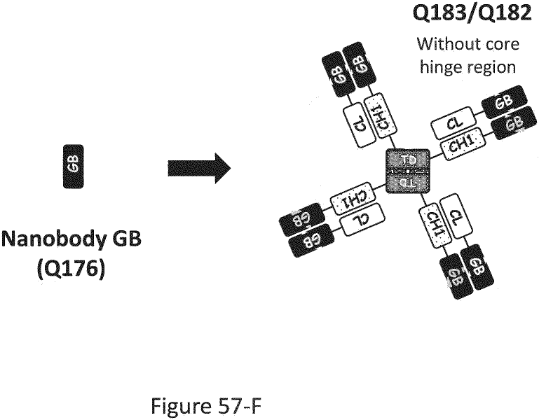

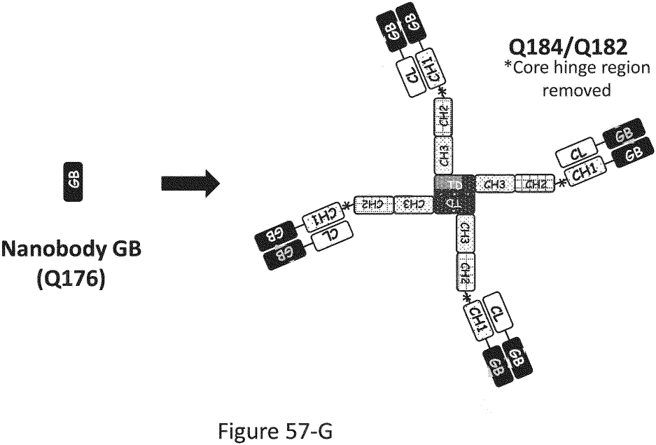

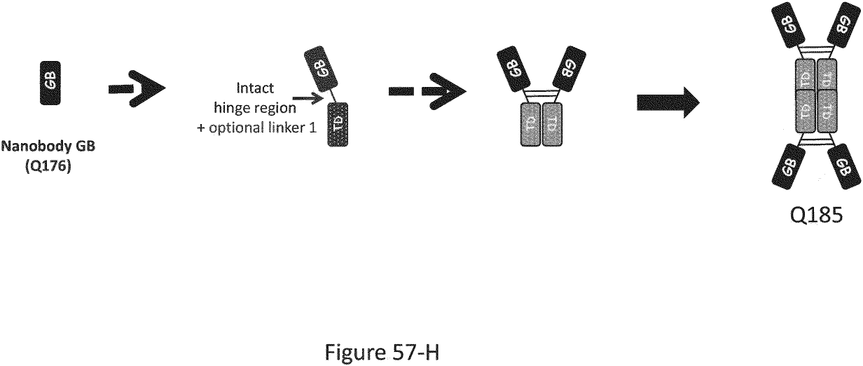

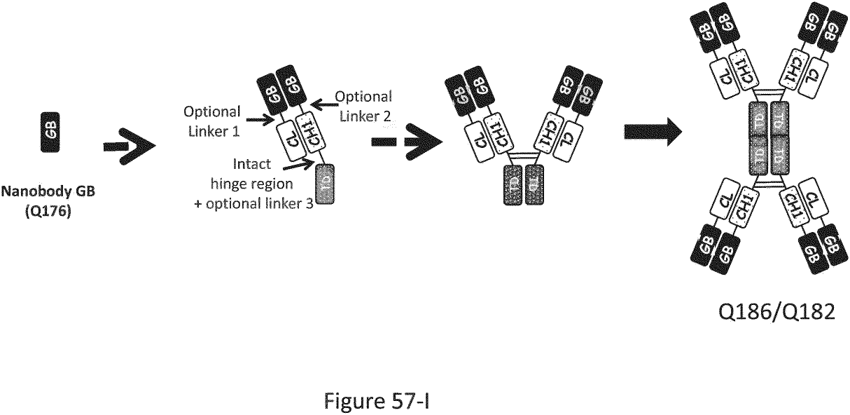

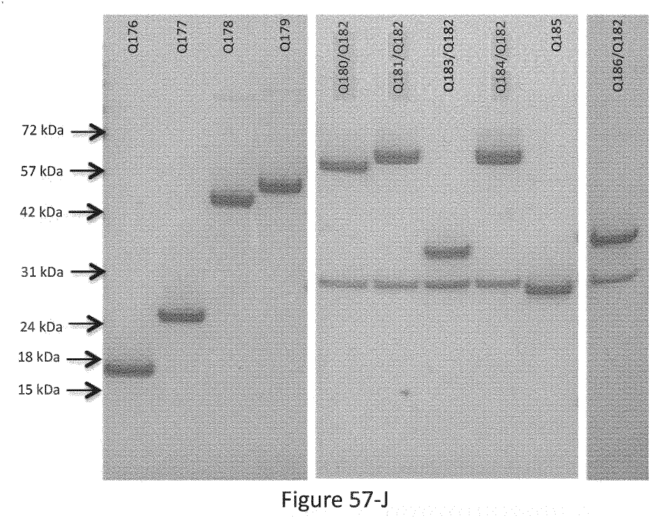

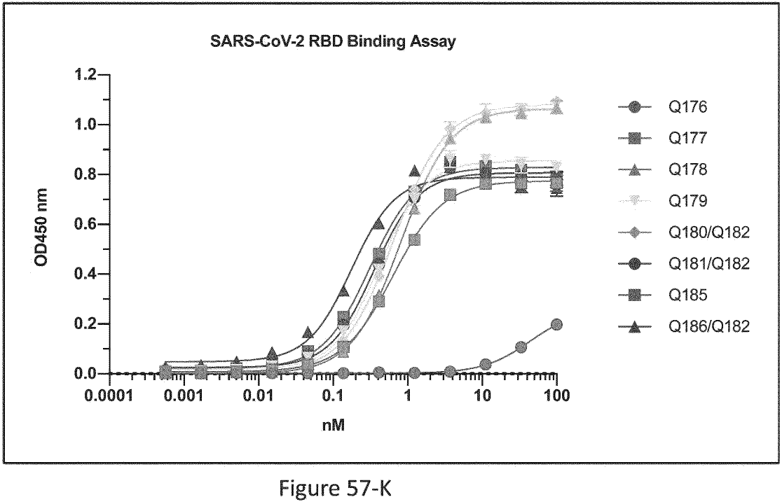

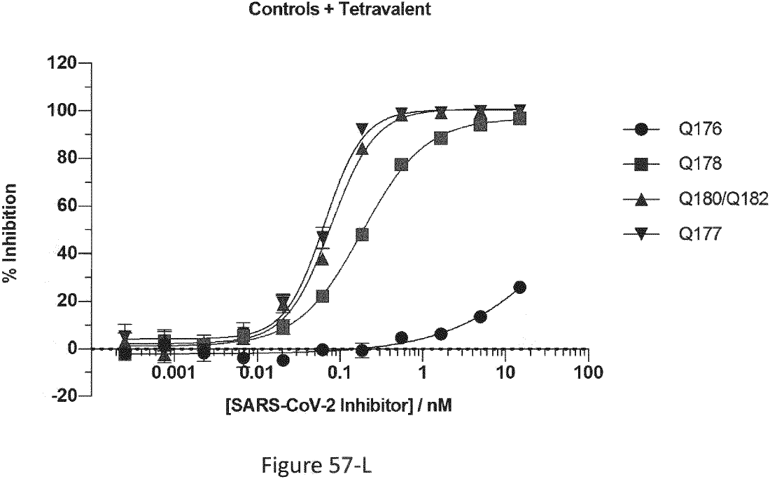

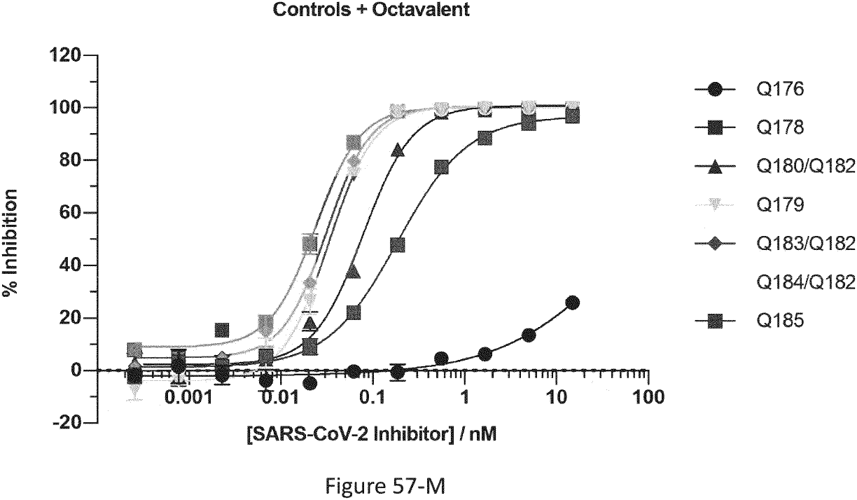

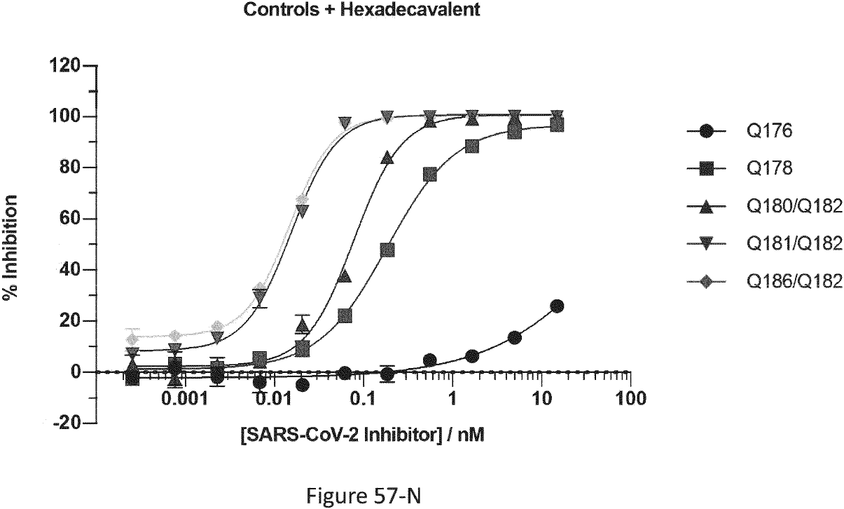

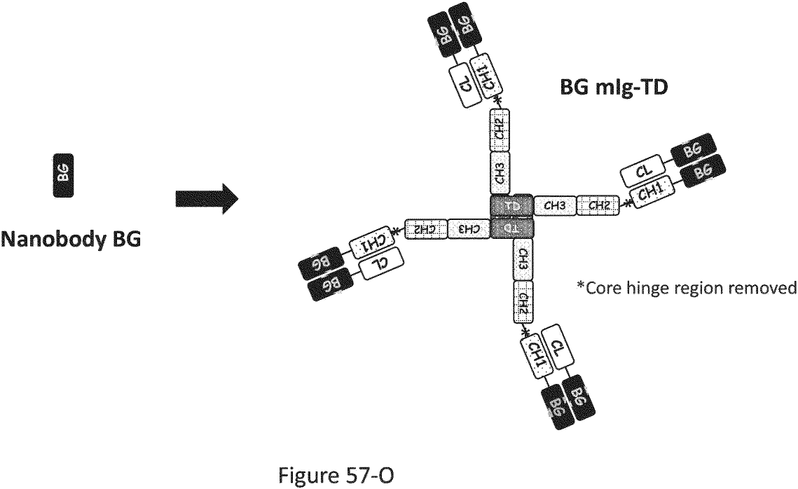

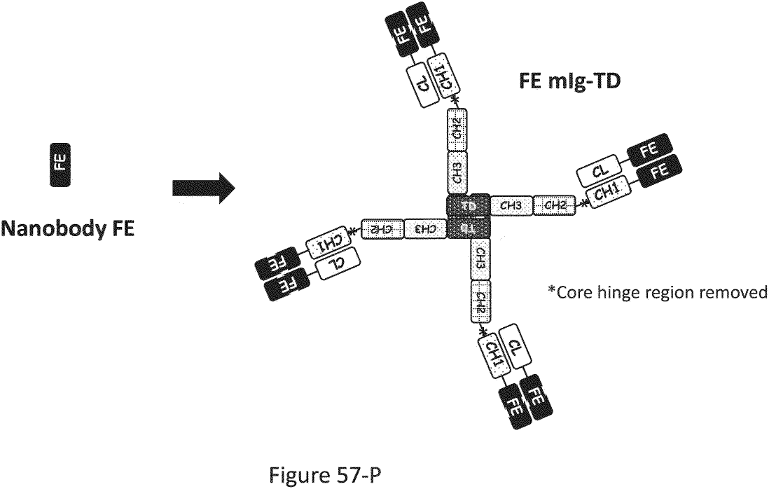

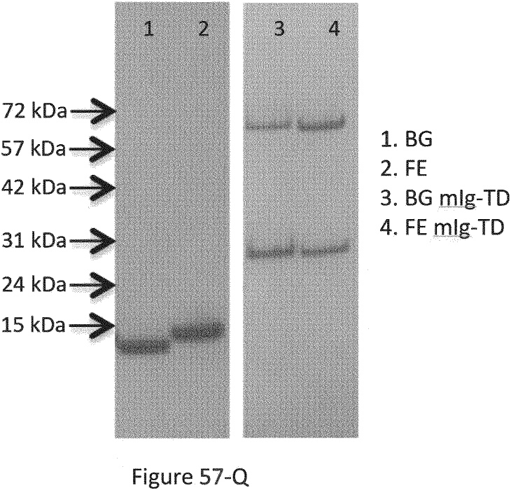

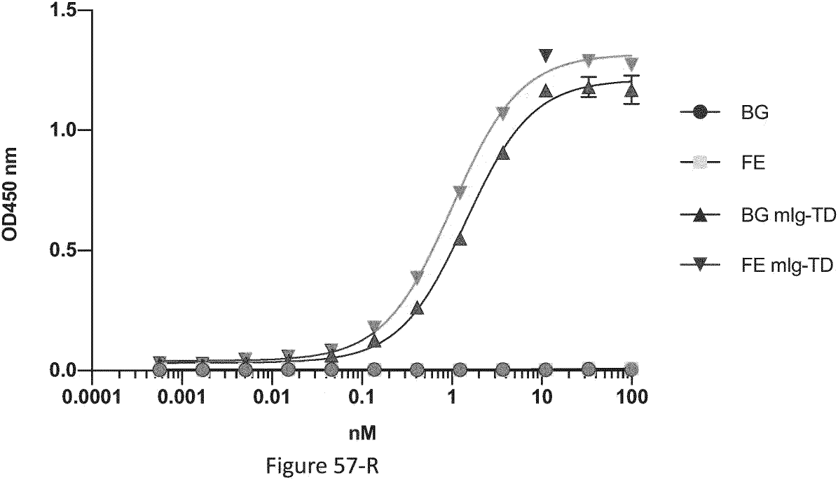

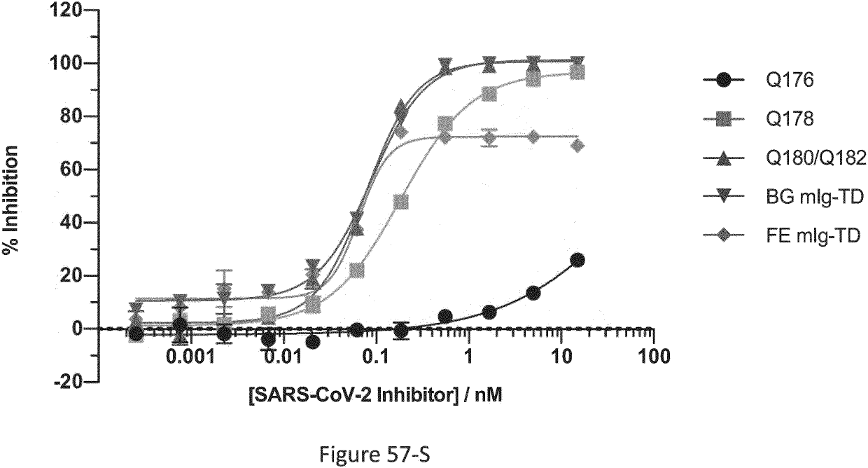

To extend the utility, SARS CoV-2 cross-reactive nanobody termed ‘GB’ (or ‘QB-GB’) was used to format different versions of Quads with varying valency, size and structure and either with or without an Fc region. The valency of the different formats varied from monovalent to octavalent as shown schematically in FIGS. 57A-I. Expression of GB based Quads were carried out in Expi293 cells and the resulting proteins were analysed on reduced SDS-PAGE gel after affinity purification (FIG. 57J). Protein polypeptides from all the expressed proteins migrated according to their expected molecular weight confirming correct expression and high purity.

Next the GB Quad proteins were analysed for their ability to bind CoV-2 RBD in an indirect ELISA binding assay. ELISA plates were coated with 200 ng CoV-2 RBD protein and serially diluted GB Quad proteins from 100 nM was added to the wells. Binding of GB Quad proteins to CoV-2 RBD was detected using anti-FLAG HRP. With the exception of GB VHH (monovalent), which only showed very weak binding at the highest concentration (100 nM), all other GB-based Quad proteins showed a dose dependent binding to CoV-2 RBD (FIG. 57K). It is noteworthy, in this binding assay, due to high avidity of these molecules and the limitations in the assay window; the difference in binding strengths between the molecules could not be differentiated.

To tease out the difference in binding strength and to demonstrate the difference in the neutralization potencies between the different GB Quad molecules, a competitive ELISA was performed. ELISA plates were coated with 250 ng of recombinant ACE2 Ig fusion protein and serially diluted GB proteins from 15 nM with a fixed amount of CoV-2 RBD (2 nM) was added to the coated ELISA plate. Wells with only CoV-2 RBD protein added without GB protein was used as 100% binding. Europium labelled streptavidin was used to detect the amount of CoV-2 RBD protein that was bound to ACE2. The data were plotted as percentage inhibition of ACE2:CoV-RBD interaction in three groups (FIGS. 57L-N). GB VHH (Q176) and the non-Quad based GB formats (Q178 and Q180) were used in each plot as reference for comparison. In general, it can be seen the neutralization potential of GB proteins were significantly increased with increasing GB binding domain valency of Quad formats with the most potent being Q186, which is a -octavalent GB Quad with an IC₅₀ value of 14.8 pM.

As part of this experiment, two additional nanobodies termed ‘BG’ and ‘FE’ (also known as ‘QB-BD’ and ‘QB-FE’ respectively) were selected for testing as Quad formats to bind and neutralize SARS-CoV-2. Nanobody BG and FE were produced as monovalent VHH and also reformatted into octavalent monomeric Ig-TD format as schematically represented in FIG. 57-O and Q. Combinatorially, they were tested for their ability to bind SARS CoV-2 RBD in an indirect binding ELISA and also for their ability to neutralize the interaction of ACE2:CoV-2 RBD in a competitive ELISA (FIGS. 57R-S). BG and FE VHH nanobodies were unable to bind RBD even at the highest concentration (100 nM) as a monovalent nonobody. The octavalent Quad formats of these two nanobodies bound CoV-2 RBD very well in a dose dependent manner and BG and FE appear to bind CoV-2 RBD in a similar manner.

A competitive ELISA was performed only with the monomeric Ig-TD formats for BG and FE as described above and the non-Quad formats of GB VHH were included for comparison (FIG. 57S). Both Quad version of the nanobodies were able to inhibit ACE2:CoV-2 interaction but only BG mIg-TD Quad was able to inhibit the interaction completely. FE mIg-TD appeared to inhibit a maximum of 70% of the ACE2: CoV-2 interaction similar to that observed with CR3022 based Quads outlined in Example 28.

All the IC₅₀ values from the competitive ELISA are summarized in Table 8.

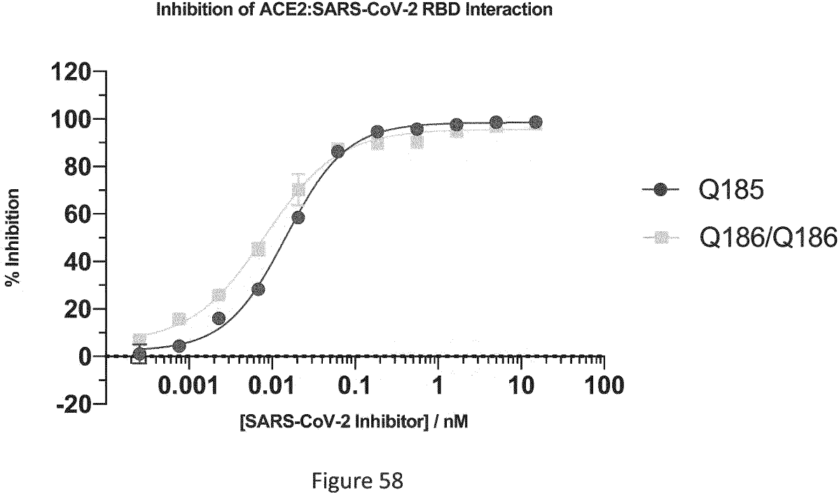

The exemplification of nanobody based Quad reformatting into multiple different formats highlighted the power of increasing binding domain valency has on the neutralization potency. In the competitive ELISA described above, the limited assay window together with the detection limitations prevented accurate measurements of some the most potent Quads such as the octavalent Quads causing rapid signal saturation. As a means to improve the assay signal and tease out the enhanced potency of two of the most potent GB-based Quads further, a competitive sandwich ELISA was performed with substantially reduced amounts of CoV-2 RBD similar to that reported by Hansen et al. In their sandwich ELISA a capture antibody was used to capture his-tagged ACE2 protein followed by the addition of a reduced amount of CoV-2 RBD (10-15 pM) plus serially diluted CoV-2 inhibitor antibody. To replicate a similar assay, ELISA plates were coated with 100 ng anti-human IgG to capture 200 ng of recombinant ACE2 IgG. Then to each well a fixed amount of CoV-2 RBD (0.5 nM) together with serially diluted CoV-2 inhibitor antibody (Q185 or Q186/Q182) was added. Europium labelled streptavidin was used to detect the amount of CoV-2 RBD protein that was bound to ACE2. In this assay set-up, the assay window was improved and the IC₅₀ value for Q185 and Q186/Q182 (FIG. 58) when compared to the lead SARS-CoV-2 antibody molecules reported by Hansen et al., indirectly confirm that both Q185 and Q186/Q182 Quads are significantly more potent than these SARS-CoV-2 neutralizing antibodies currently under clinical investigation.

### Example 30: Engineering of Multivalent SARS CoV-2 Neutralizing VHH Nanobody Linked in Tandem Into Different Quad Formats With Increasing Valency and Potency

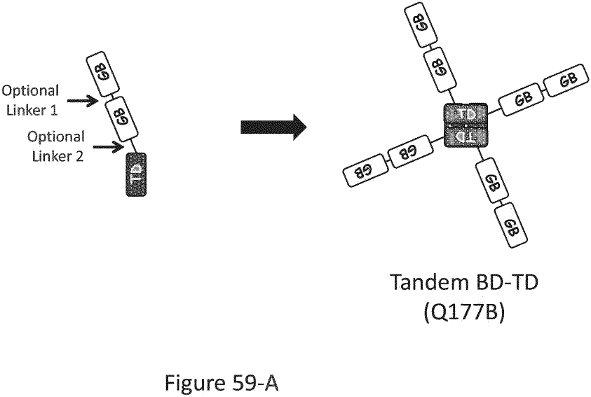

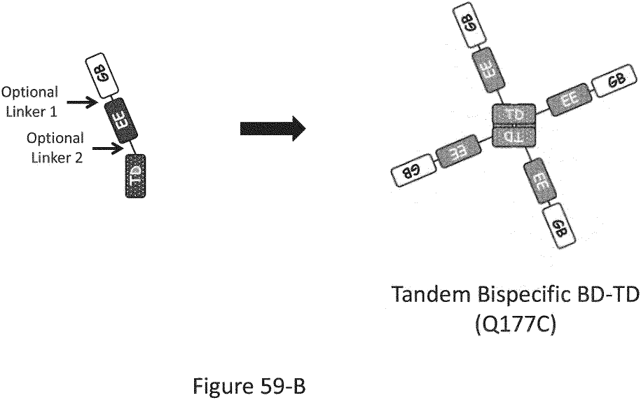

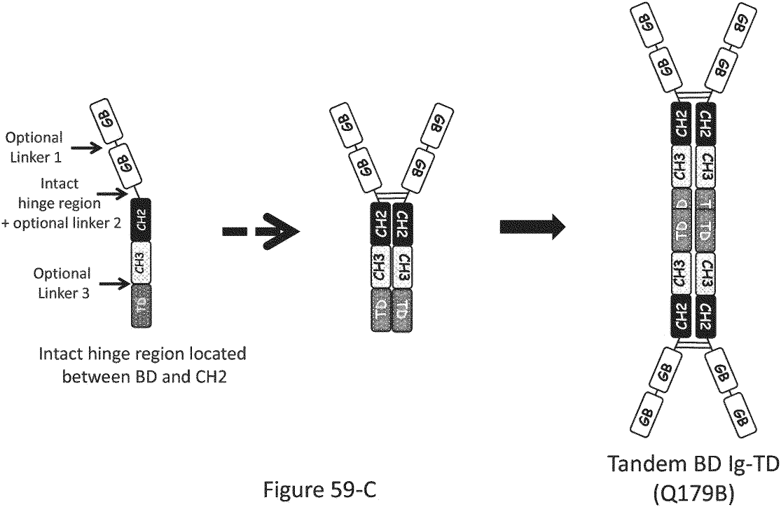

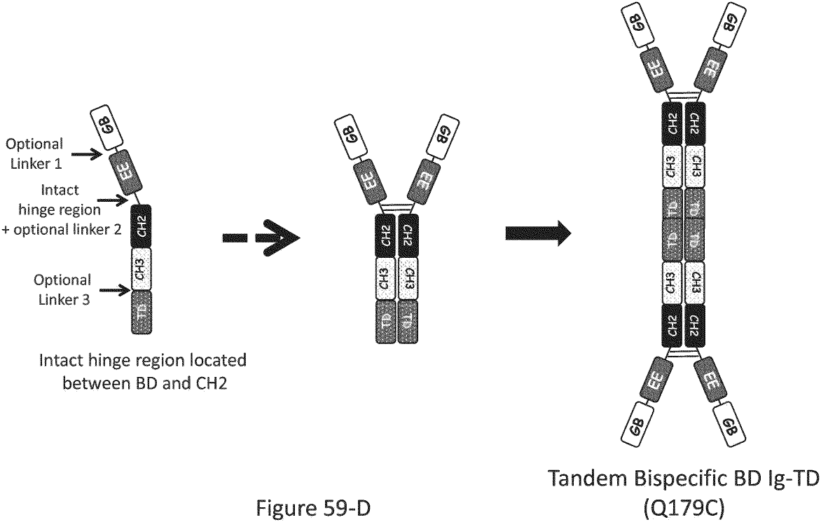

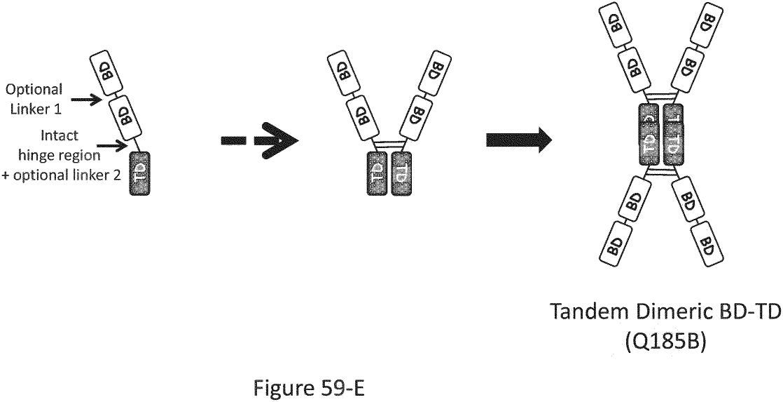

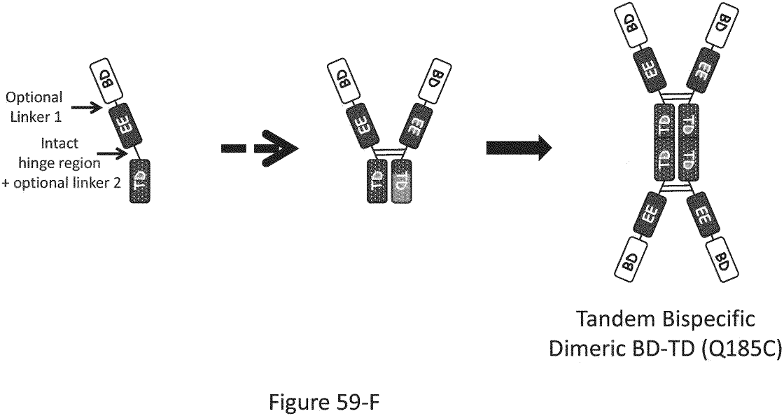

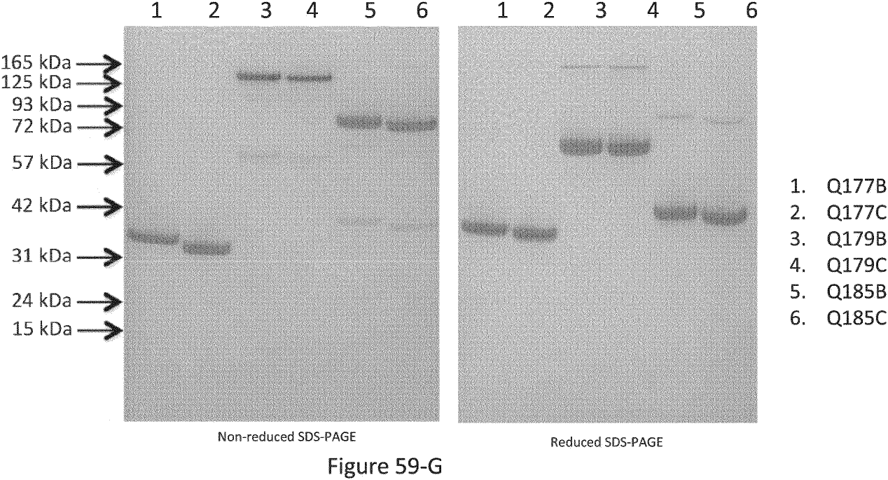

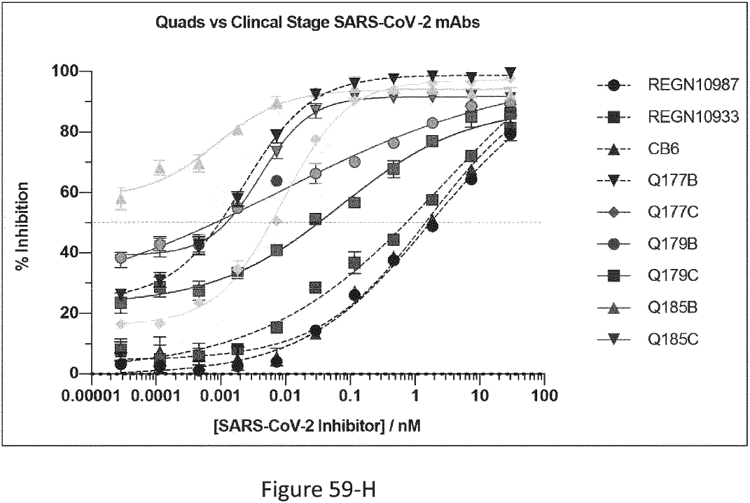

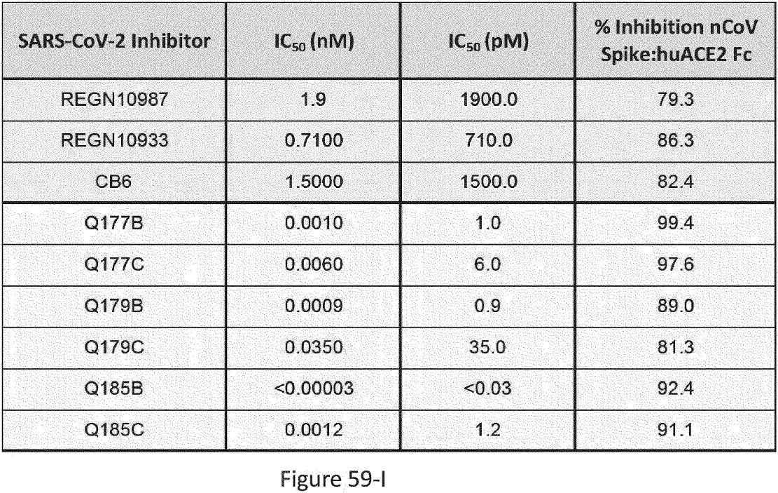

In example 29, SARS CoV-2 cross-reactive nanobody ‘GB’ was formatted into different multivalent formats with varying size, shape and valency. In this example, the same SARS CoV-2 cross-reactive ‘GB’ nanobody was used to generate multivalent Quads where the VHH was linked in tandem via a flexible peptide linker as shown schematically in FIG. 59-A(Q 177B). In a similar strategy, a bispecific VHH Quad version was also generated by linking in tandem SARS-CoV-2 VHH ‘GB’ with a VHH with cross-reactivity to MERS-CoV RBD termed ‘EE’. The bispecific VHH tandem Quad is shown schematically in FIG. 59-B(Q177C). In both of these examples, the tandem VHH is linked directly to the TD via a linker, which is rapidly assembled to form tetramers with valences of 8 for the monospecific Quad and 4 + 4 for the bispecific Quad format.

Additional tandem VHH Quad formats were generated either as monospecific containing only ‘GB’ linked in tandem (FIG. 59-C(Q179B) and 59-E (Q185B)) or as bispecific VHH formats containing ‘GB’ and ‘EE’ VHHs linked in tandem (FIG. 59-D(Q179C) and 59-F (Q185C)). In both of these examples, the Ig core hinge region was kept intact allowing the formation of a homodimer intermediate. This homodimer intermediate is then rapidly dimerized to form a dimer of a dimer (tetramer) facilitated through tetramerization of the TD.

The tandem VHH Quad formats generated in this example were expressed in Expi293F cells and the resulting proteins were affinity purified directly from the culture supernatant as described in example 29. The purified proteins were analysed by SAS-PAGE as denatured non-reduced protein and reduced protein (FIG. 59-G). The proteins separated out on SAS-PAGE as non-reduced protein confirmed these Quads were highly pure with purity >95%. Further, the molecular weights of the different tandem Quad formats were also confirmed as the protein all separated out in SDS-PAGE according to their expected molecular weight. The reduction of the cysteine bonds present in the core hinge region of Quad formats Q179B, Q179C, Q185B and Q185C can be seen in the reduced SDS-PAGE where the molecule weigh of these Quads are reduced down from the dimeric intermediate state to the monomeric state.

To further analyse these tandem VHH Quad formats, their enhanced potential to neutralize the interaction of SARS-CoV-2 spike protein with the ACE2 receptor, a competitive ELISA assay was performed as detailed in example 29 with the following conditions. High binding ELISA plates were coated with 100 ng of anti-human Ig, which was used to capture 100 ng of ACE2-Fc. A fixed amount of SARS-CoV-2 RBD biotinylated protein at 15 pM was pre-mixed for 30 mins with serially diluted 1 in 3 folds of anti-SARS-CoV-2 tandem VHH Quads from 30 nM before adding to the ELISA plate with the captured ACE2-Fc. Any SARS-CoV-2 RBD not competed by the Quad molecules were detected using anti-Strep-HRP detection antibody. The signal obtained from the wells with only RBD added without any competitor was used to determined 100% binding and wells with no added RBD or competitor molecule was used to determine zero binding. From this, the percentage inhibition of SARS-CoV-2 RBD interaction with ACE2 at different competitor concentration was plotted (FIG. 59-H) and their potency was determined as summarized in FIG. 59-I. As a reference comparator, three clinical stage SARS-CoV-2 antibodies with potential to compete the interaction of SARS-CoV-2 spike with ACE2 receptor (Hansen et al., 2020; Shi et al., 2020) were also included in this assay to determine the relative potency enhancement of the VHH tandem Quads.

All of the tandem VHH Quads were found to be significantly more potent at neutralizing SARS-CoV-2 RBD interaction with ACE2 than the three clinical stage anti-SARS-CoV-2 antibodies. The most potent of the tandem VHH Quad (Q185B) with IC₅₀ value of 0.03 pM was found to be between 23,000 - 63,000 times more potent than the three clinical stage antibodies in this competitive ELISA assay setting. The massive improvement in neutralization potency using tandem VHH to generate Quads clearly confirms the multivalent strategy is a good approach for improving antibody functionality. This opens up the scope to further developing Quad formats with even higher neutralization potencies.

### Example 31: Further Engineering of Multivalent SARS CoV-2 Neutralizing VHH Nanobody Linked in Tandem Into Different Quad Formats With Increasing Valency and Potency

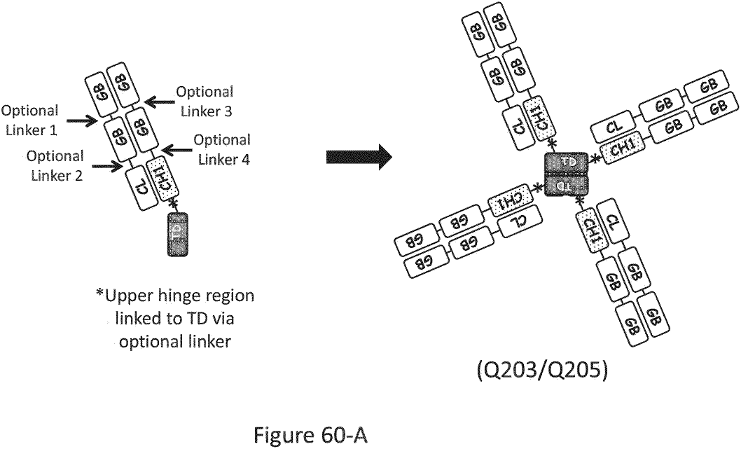

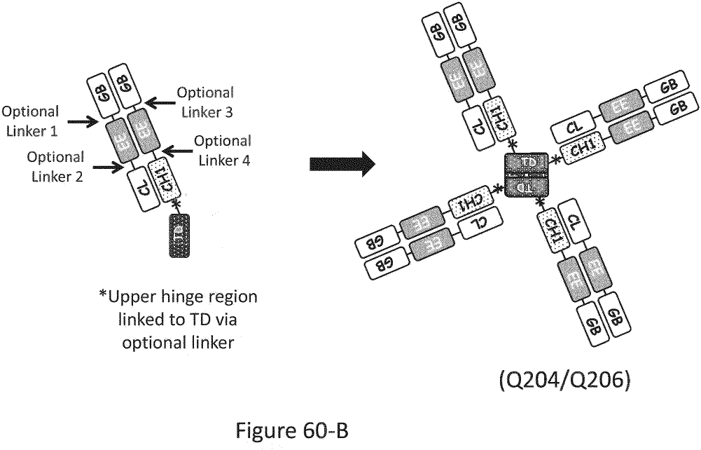

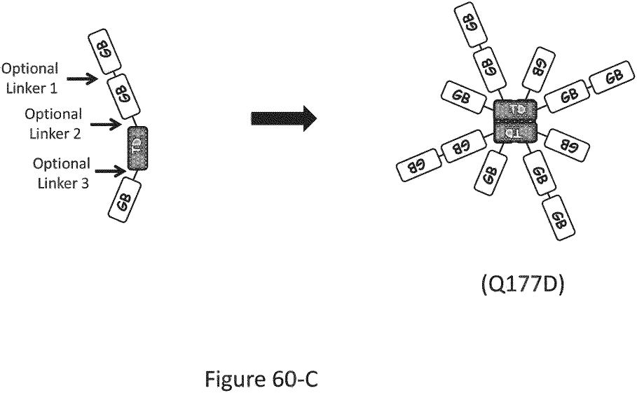

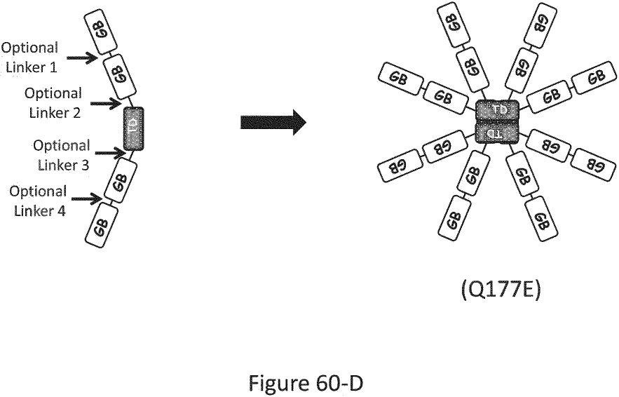

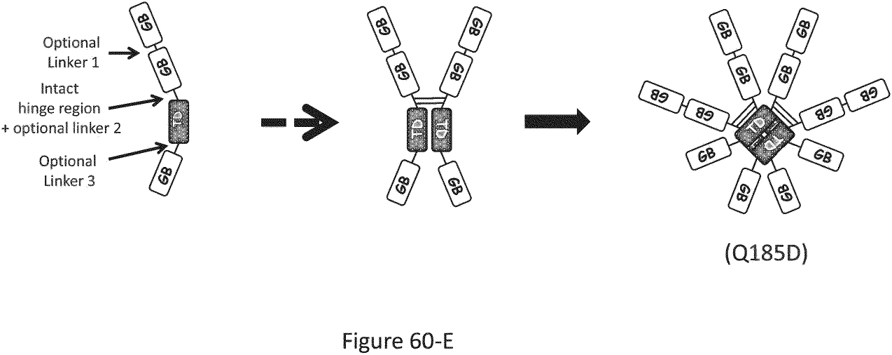

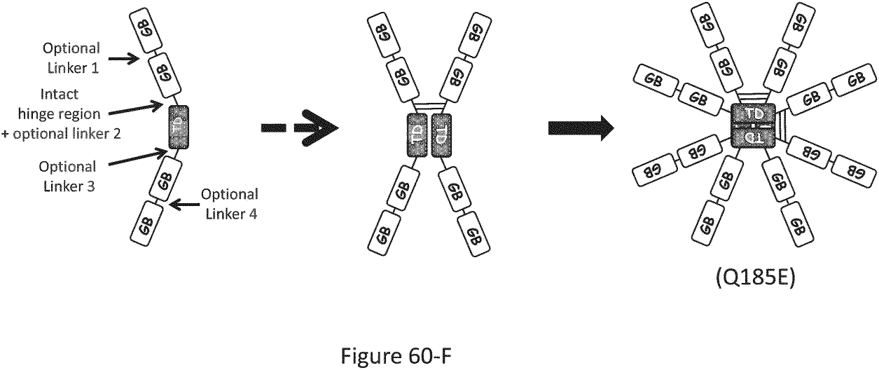

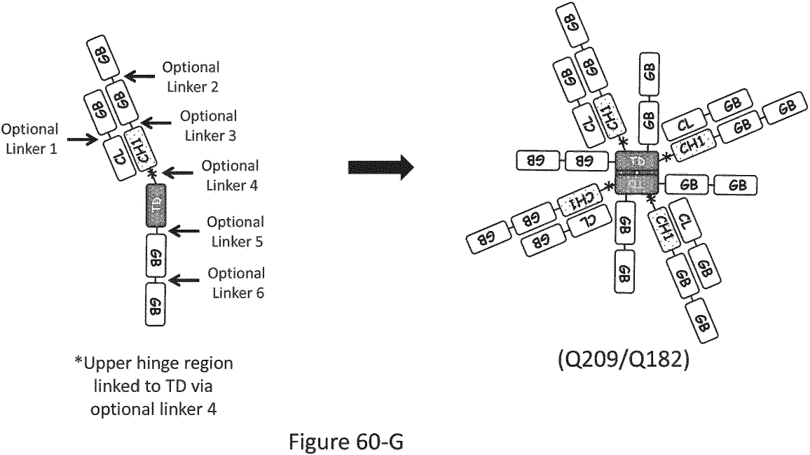

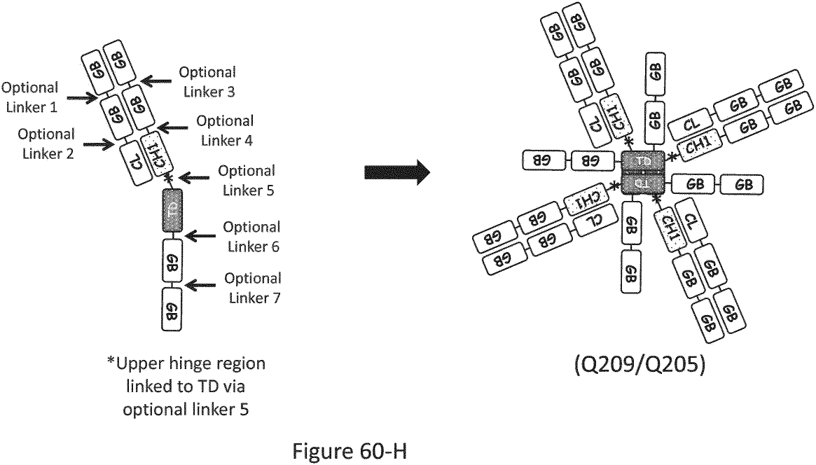

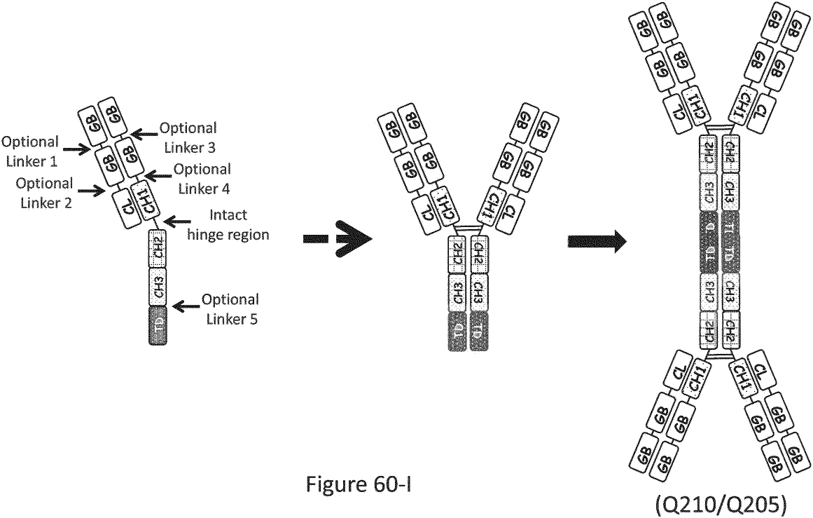

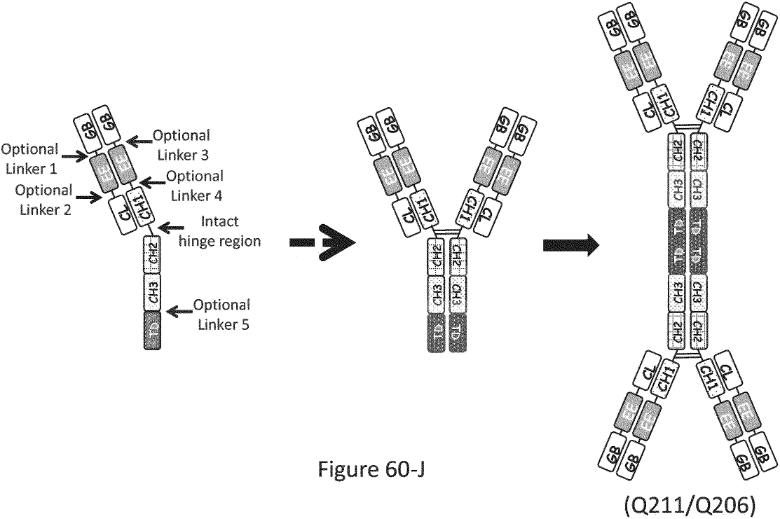

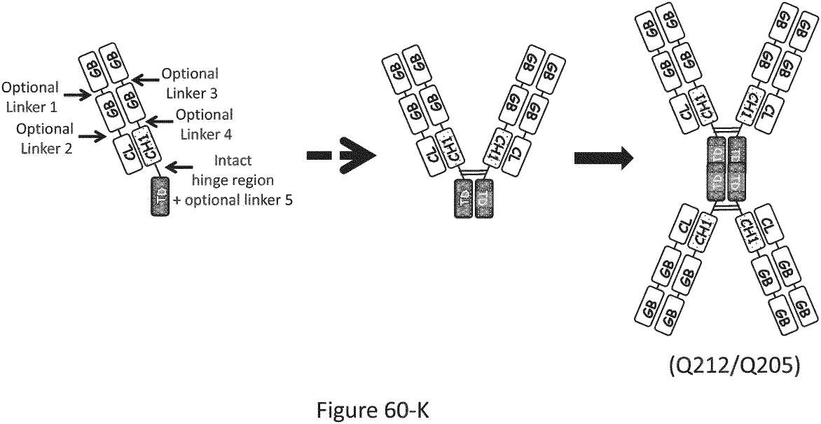

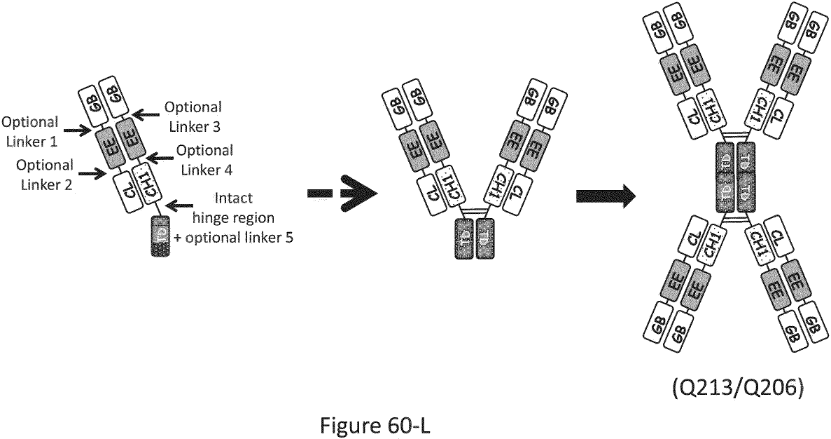

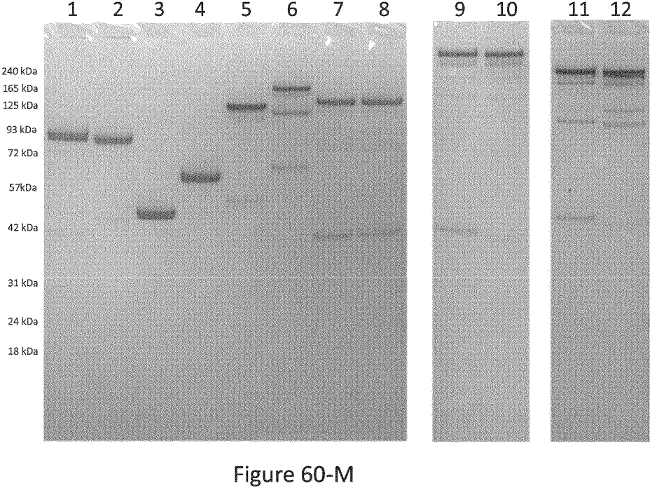

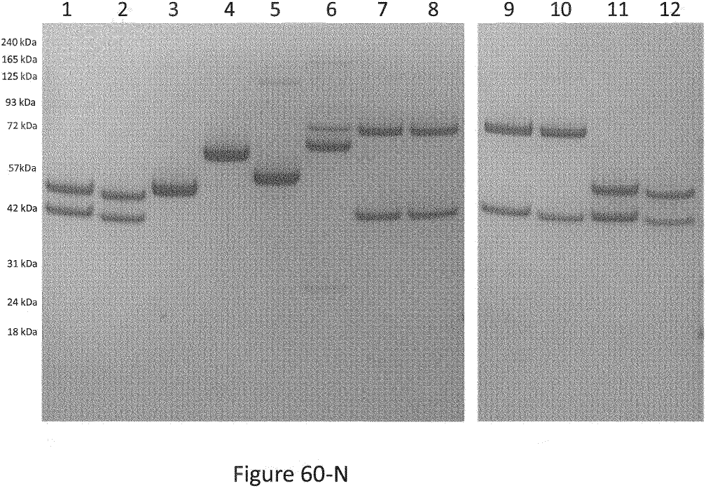

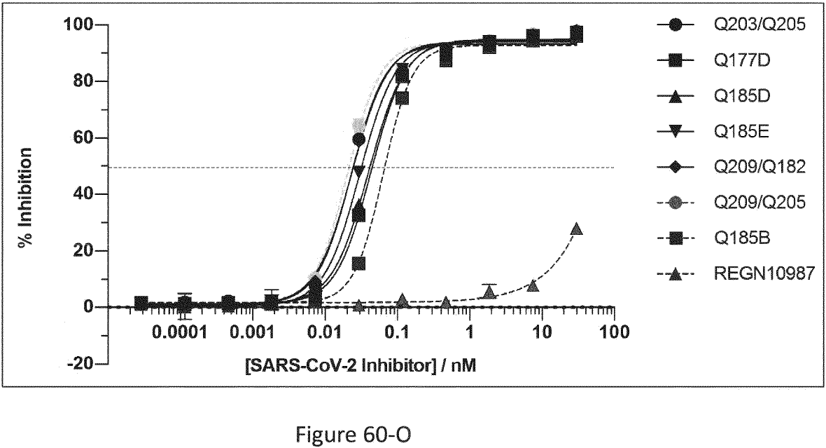

To extend the multivalent Quad formats beyond octavalency using tandem VHH, in this example twelve additional Quad formats were generated utilizing either the tandem ‘GB’ VHH or ‘GB’ and ‘EE’ VHH linked in tandem to generate monospecific and bispecific Quad formats respectively. The different Quad formats varied in shape, size and valency as schematically represented in FIGS. 60-A - 60-L. The valency of these multivalent Quad formats ranged from 12 -24 for the monospecific Quads (Q203/Q205, Q177D, Q177E, Q185D, Q185E, Q209/Q182, Q209/Q205, Q210/Q205 & Q212/Q205) and 16 in an 8 + 8 configuration for the bispecific Quad formats (Q204/Q206, Q211/Q206 and Q213/Q206).

The multivalent tandem VHH Quads were expressed in Expi293T cells as described in example 29. Surprisingly and like all of the previously described Quads formats, these tandem VHH formats all expressed well with high purity as soluble secreted protein with average protein titres >150 mg/L. Following affinity purification with Ni-NTA, the proteins were analyzed by SDS-PAGE with either non-reduced or reduced protein. All of the Quad proteins separated out on the non-reduced SDS-PAGE (FIG. 60-M) according to their expected molecular weight corresponding to the monomeric form or according to their dimeric form where Quad formats are dimerized through the hinge region such as Q177E. The SDS-PAGE separating the reduced protein samples (FIG. 60-N) nicely highlights the reduction in disulphide bonds in Quad formats containing two chains such as Q211/Q205 where the two chains are held together via disulphide bonds. Protein reduction can also be seen as expected in Quad formats with an intact hinge region containing disulphide bonds such as Q185E. By demonstrating protein reduction either by separating the two chains or by reducing a dimer into a monomer in Quad formats containing core hinge region confirms these Quad formats are assembled correctly.

Given the further increase in binding domain valency in the Quad formats in this example compared to those in example 30, the expectation is that the neutralization potency would also be further improve. To demonstrate the potency enhancement, competitive ELISA was performed using a selection of Quad proteins with increased stringent condition. The competitive ELISA was performed similarly to that described in example 30 with the exception that 2 nM fixed amount of biotinylated SARS-CoV-2 RBD was used instead of 15 pM. The most potent Quad molecule described in example 30 (Q185B) along with the clinical stage mAb ‘REGN10987’ was also run alongside these Quads for comparison (FIG. 60-O). From the initial observation, it is noteworthy the clinical stage mAb ‘REGN10987’ at these stringent assay conditions does not appear to be very potent compared to the multivalent Quad formats. For example at 1 nM concentration of anti-SARS-CoV-2, REGN10987 has almost zero neutralization potential whereas all of the Quad formats at 1 nM concentration can almost completely neutralize SARS-CoV-2 RBD interaction with ACE2.

Furthermore and as expected, all of the new Quads tested in this example (Q203/Q205, Q177D, Q185D, Q185E, Q209/Q182 and Q209/Q205) showed improved neutralization potency compared to Q185B, which was found to be the most potent Quad in example 30. However, the extent of the improvement in potency between the new Quad formats highlighted in this example and Q185B was not observed as substantial given the binding domain valency difference between for example Q209/Q208 and Q185B being 24 and 8, respectively. The most likely explanation for this is probably due to the limited assay sensitivity and therefore any significantly gain in potency could not be differentiated due to the rapid saturation of the assay signal. To determine the true potency of these multivalent Quad formats, which are expected to be ultra-potent would warrant further investigation of these Quads in an in vivo setting where a measure of a biological effect rather than a assay signal would be measured and thus making it possible to differentiate and observe their true potency enhancement.

### Example 32: Improving SARS-CoV-2 Neutralization Potency of Clinical Stage mAbs by Reformatting Them Into Multivalent Quad Formats

The first-in-class clinical stage anti-SARS-CoV-2 mAbs REGN10987, REGN10933 (Hansen et al., 2020) and CB6 (Shi et al., 2020) are currently being trialed for their efficacy in reducing viral load and viral infections in COVID-19 patients. Given the results highlighted in the above examples whereby increasing the binding domain valency, a consistent improvement in neutralization potency could be observed in the in vitro neutralization assays. Further as shown in example 31, REGN10987 could not neutralize SARS-CoV-2 RBD binding to ACE2 using the stringent assay conditions compared to the multivalent Quads. In this example, the binding domains of REGN10987, REGN10933 and CB6 were used to reformat them into three different multivalent Quad formats to show 1) How the different formats can neutralize the SARS-CoV-2 binding to ACE2 and 2) Whether the different formats effect the neutralization potential due to their size and structural configuration.

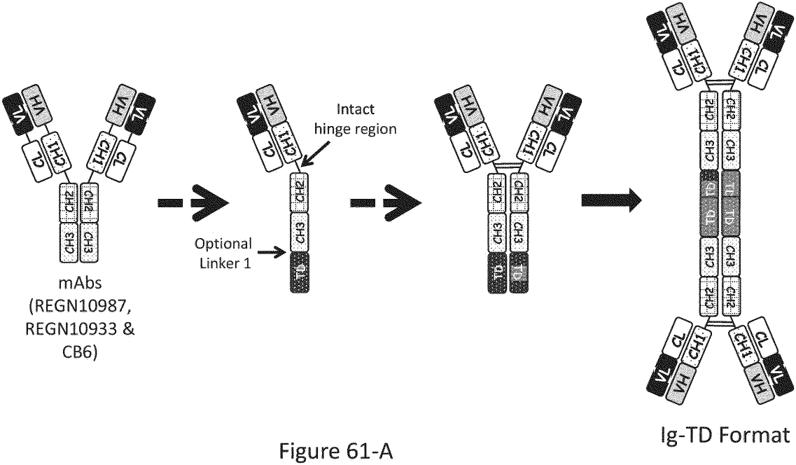

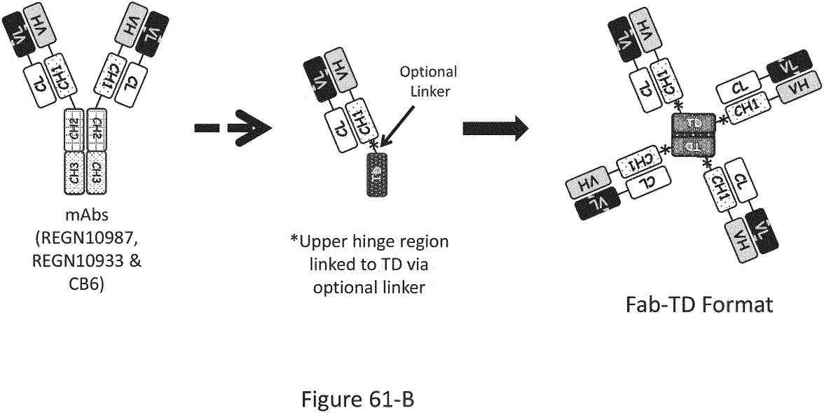

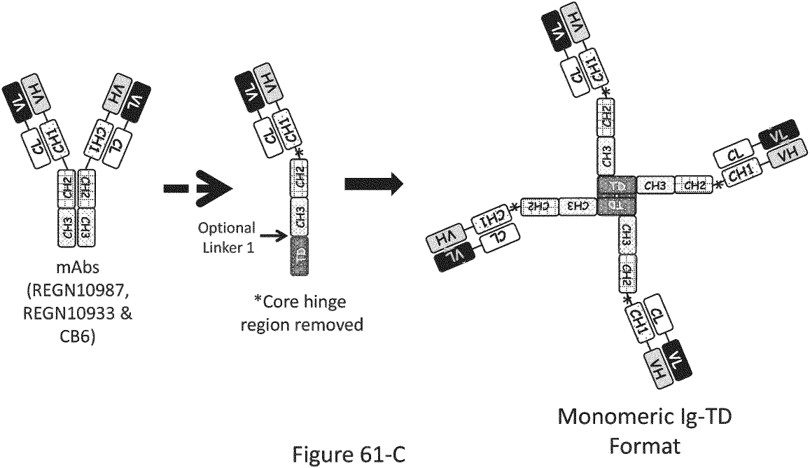

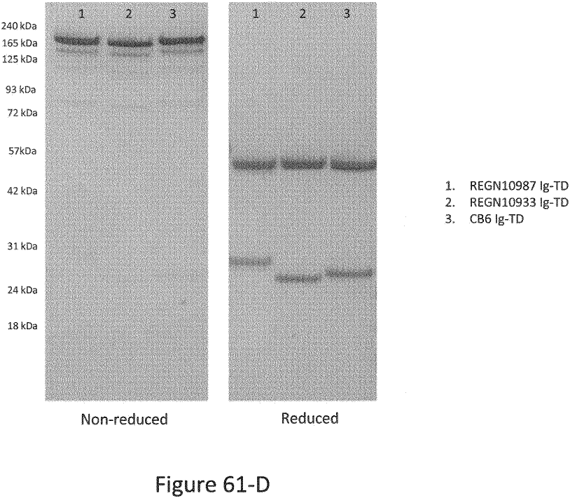

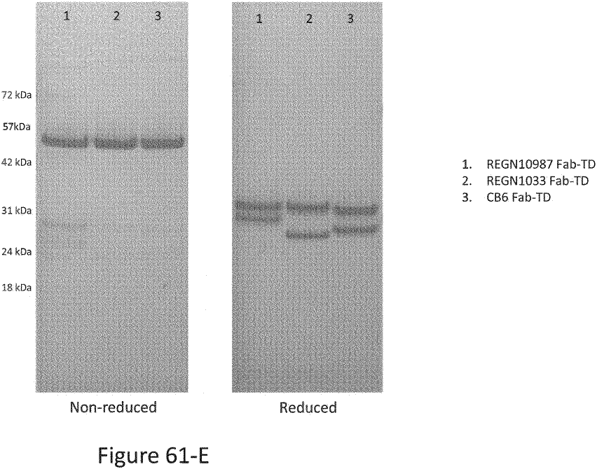

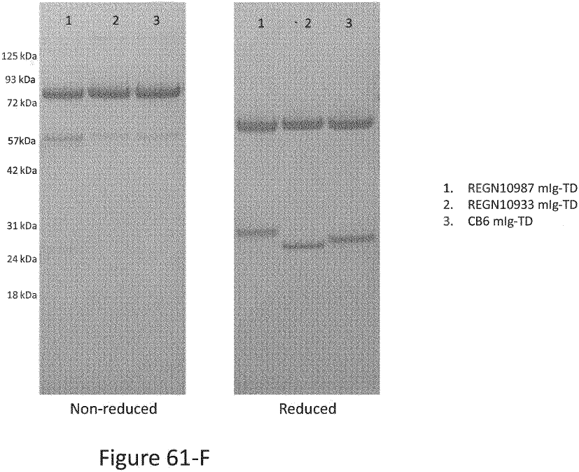

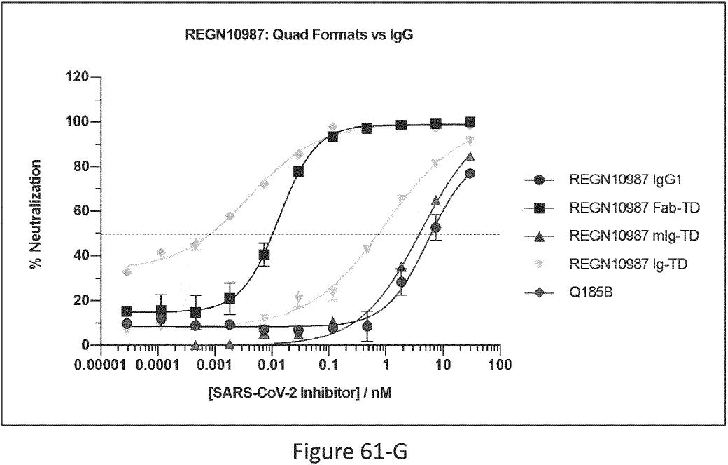

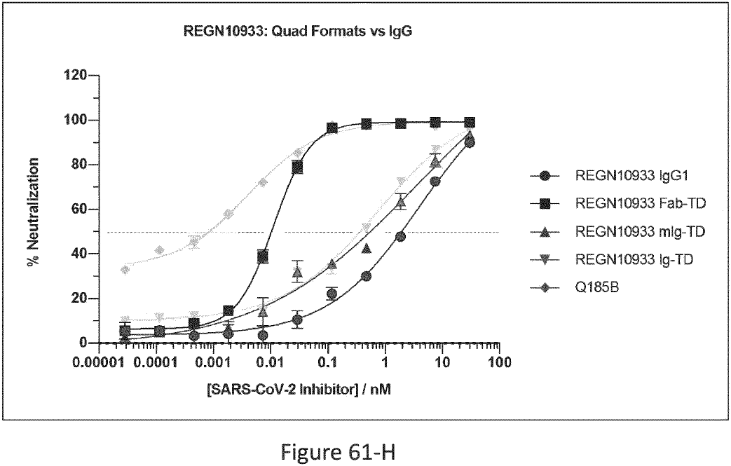

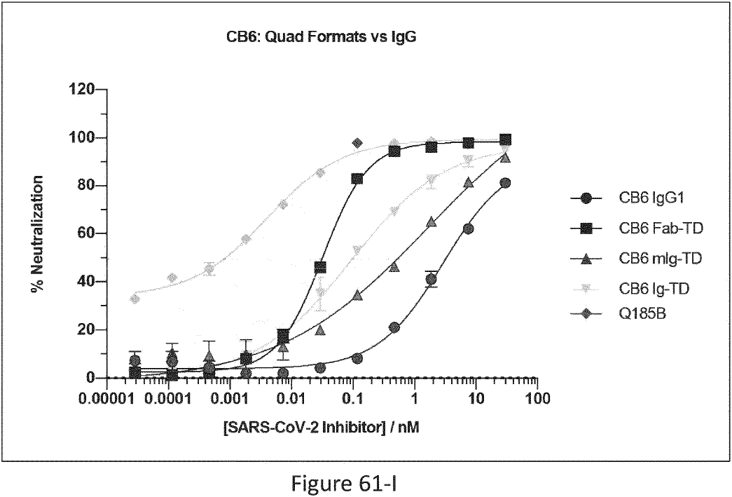

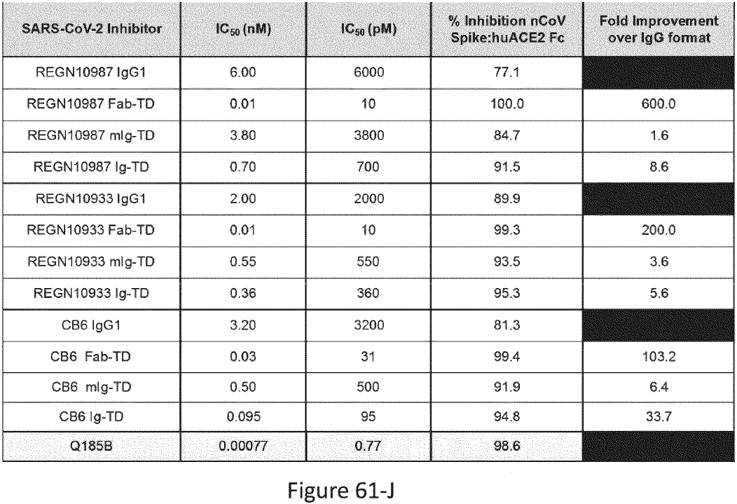

In the first format termed Ig-TD, the p53 tetramerization domain was directly linked to the C-terminus of the mAb with the CH3 domain as schematically shown in FIG. 61-A. In the second format termed Fab-TD, the TD was directly linked to the upper hinge region but lacked the core hinge. In this example, the upper hinge region was directly linked to TD as shown schematically in FIG. 61-B. The third format is similar to the Ig-TD format except the core hinge region only was removed where the upper and lower hinge region was kept intact to give a monomeric Ig-TD format as shown schematically in FIG. 61-C. In all three examples, the TD facilitated the assembly of a tetramer and thus transforming the bivalent mAbs into tetravalent mAbs.

The three different Quad formats of REGN10987, REGN10933 and CB6 were expressed in Expi293T cells. Like normal IgG antibodies, the Quad formats of these mAbs also expressed as soluble secreted proteins where the assembled tetrameric Quad proteins were harvested from the culture supernatant and affinity purified. The purified proteins were separated out on SDS-PAGE gels using either non-reduced or reduced proteins (FIGS. 61-D - 61-F). From the non-reduced SDS-PAGE, the Quad proteins appear to be highly pure (>95%) and in the reduced SDS-PAGE, reduction of the two chains can be seen, which confirms these Quad proteins were correctly assembled.

To check for improved neutralization potency of the Quad formats over the parental IgG format, competitive ELISA was performed as described in example 29 with some changes in the assay conditions. Instead of SARS-CoV-2 RBD, biotinylated SARS-CoV-2 Spike trimer was used at a fixed concentration of 75 pM. For each of the clinical stage mAb, the three different Quad formats were compared for their neutralization potential compared to the parental IgG mAb and against Q185B as highlighted in FIGS. 61-G - 61-I. From the initial observation, it can be seen that all of the Quad formats had improved neutralization potency of SARS-CoV-2 spike interaction with ACE2 compared to the parental mAb but not as potent as the tandem VHH Quad, Q185B. Secondly, it can be seen the Ig-TD and mIg-TD Quad formats only had moderate enhancement in neutralization potency whereas the Fab-TD format consistently showed superiority for all three clinical stage mAbs in terms of improving the neutralization potency compared to the parental mAb as summarized in FIG. 61-J. For example, REGN10987 Fab-TD format had a 600-fold improvement in neutralization potency over the parental mAb. From these data in this artificial in vitro neutralization assay setting, it can be seen that the size, shape and even the flexibility of the molecule could enhance the neutralization potential of inhibiting SARS-CoV-2 Spike protein binding to ACE2. The superior neutralization potency enhancement of Fab-TD format is something worth analyzing generally for antibodies, such as further to improve other SARS-CoV-2 antibodies binding alternative epitopes within the spike protein with improved binding such as antibodies targeting the S2 ectodomain.

### Example 33: Design, Expression and Analysis of Multivalent VHH Nanobody Formats Based on Sequence Nb-112

As described in previous examples, VHH nanobodies can be rapidly designed into different multivalent formats starting from simple monomeric building blocks. The VHH sequence Nb-112 against SARS-CoV-2 spike protein described previously (Esparza et al., 2020) was used to design different multivalent formats with varying size, shape, flexibility and valency. FIGS. 62A & 62B depicts two such examples that were produced in this example where VHH Nb-112 was reformatted to yield tetravalent and octavalent Quad molecules. Nb-112 sequence could also be easily reformatted into any of the Quad formats described in the previous examples and as schematically shown in FIGS. 55-60. Optional Linkers 1 and 2 (GGGGSGGGGS and GGSGGS respectively) shown in FIG. 62B were included in the octamer we constructed. We included GGGGSGGGGS Linker 1 in the tetramer we constructed (FIG. 62A). It is possible in alternatives to omit linkers, or to use one linker (linker 1 or 2) in the octamer polypeptide.

One of the advantages of these multivalent Quads, apart from them being highly soluble, stable and amenable to large-scale production, is that they are relatively small in size, particularly the tetravalent versions without Fc compared to standard mAbs. The relative small size would be particularly advantageous when formulating these molecules for delivery through inhalation. Owing to their high stability properties, Quad based nanobodies is likely to maintain the structural integrity and thus the functional properties to neutralize SARS-CoV-2 after nebulization. Further, given the size of these Quads being above the renal clearance threshold, Quads delivered through inhalation is likely to persist for a longer period and provide a molecule with significantly enhancement efficacy than the native VHH Nb-112 nanobody.

The tetravalent (also called Q232) and octavalent (also called Q233) versions of Nb-112 were produced as schematically represented in FIGS. 62A and B for which the polypeptide sequences are shown in Table 24. The monovalent Nb-112 nanobody (also called Q231 was also produced for comparison. Expression vectors for producing Q231-Q233 where transfected into Expi293F cells and the proteins were produced as described in Example 28. Since the VH of Nb-112 nanobody is a VH3 subfamily member known to have intrinsic affinity for Protein A (Seldon et al., 2011), the harvested protein from culture supernatant were purified using MabSelect Sure (Sigma). The purified Q231-Q233 proteins were analysed on denaturing SDS-PAGE (FIG. 62C-1). Protein polypeptides from all three proteins (Q231-Q233) migrated according to their expected molecular weight confirming correct expression and high purity. The protein yields after Protein A affinity purification was measured using Nanospec and it could be seen the tetravalent (Q232) and octavalent (Q233) versions of Nb-112 yielded excellent protein yields equivalent to 338 mg/L and 288 mg/L respectively (FIG. 62C-2). The monovalent version (Q231) on the other hand being the smallest of the molecule surprisingly yielded very poor protein yield equivalent to only 19 mg/L. To investigate whether this poor protein yield was due to poor expression or inadequate purification method and since a 6xhistidine tag was included onto the C-terminus, Q231 was purified using Ni-NTA column as described in Example 28. After measuring the protein yield using nanospec, the protein yields was found to be good and equivalent to 187 mg/L. This confirmed the expression of the monovalent Nb-112 (Q231) was fine and purification using Protein A resin through the monovalent interaction was not sufficient to efficiently purify this protein whereas surprisingly and advantageously the tetravalent and ocatvalent versions with enhanced binding to Protein A resin through multiple VH domain interaction with the resin allowed these multivalent proteins to be purified efficiently similar to a mAb containing an Fc region.

To investigate the functionality of the multivalent Nb-112 Quad proteins, competitive ELISA was performed for their ability to neutralize the interaction of SARS-CoV-2 spike RBD with the ACE2 receptor. The monovalent Nb-112 was reported to be a potent neutralizer of spike RBD interaction with ACE2 (Esparza et al., 2020). Therefore, two different amounts of spike RBD were used in competition ELISA. Briefly, high binding ELISA plates were coated with 100 ng/well of anti-human IgG and incubated at 4° C. overnight. After blocking with 1% BSA for 1 hour at room temperature, 100 ng/well ACE2-Fc was added and the plate incubated for a further 1 hour. The plates were washed 3 times with sample buffer (PBS containing 0.1% Tween) and 100 ul/well serially diluted anti-SARS-CoV-2 molecules (Q231-Q233 and REGN10933 as positive control) starting from 30 nM premixed at room temperature for 30 minutes with a fixed about of biotinylated SARS-CoV-2 RBD either at 2 nM (FIG. 62D) or 150 pM (FIG. 62E) was added to the coated plate. Wells with only sample buffer or only spike RBD protein added without any antibody was used to determine percentage neutralization. After incubating the plate for one 1 hour, the plates were washed again 3 times and a detection protein (Strep-HRP) was added to detect non-competed spike RBD that bound the captured ACE2 protein. After the final wash as above, the signal was generated with the addition of 100 ul TMB and the reaction was stopped with the addition of 50 ul 1 M sulfuric acid. The absorbance at 450 nm was read using a microplate reader. The data was plotted as percentage neutralization over a range of anti-SARS-CoV-2 inhibitor concentration.

From the data, a nice dose-dependent inhibition of SARS-CoV-2 interaction with ACE2 can be seen in the competitive ELISA at both the high (2 nM -FIG. 62D) and low (150 pM -FIG. 62E) amounts of SARS-CoV-2 RBD used, confirming the produced multivalent Nb-112 molecules are functional. Furthermore, an increase in potency with increase binding domain valency of the Nb-112 molecules can be seen from the IC₅₀ values. It is noteworthy, given Nb-112 is already highly potent and even more so than the clinical stage mAb (REGN10933) and given the ELISA has a limited assay window, it would not be possible to determine the true levels of potency enhancement of the multivalent Quads over the monovalent Nb-112 nanobody using this in vitro ELISA assay (it likely is even greater than measured). Nevertheless, a good correlation with increase binding domain valency with increase neutralization potency can be seen when comparing the potency between the tetravalent and octavalent versions of Nb-112 with the monovalent version. To determine the true potency enhancement of these multivalent Quads would require further characterizing in in vivo animal infection models. We expect them to be highly potent in aminals and man.

### Example 34: Design, Expression and Analysis of Multivalent VHH Nanobody Formats Based on Sequence Nb-112 With Intact Hinge Region

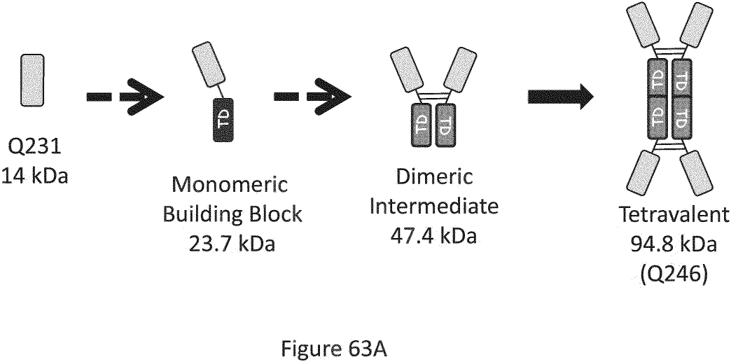

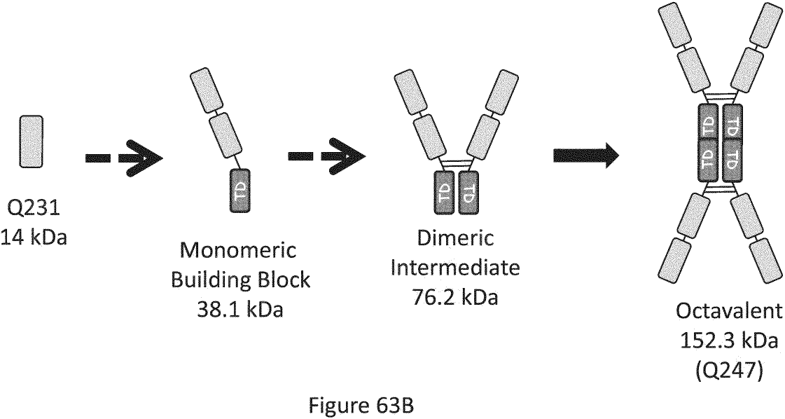

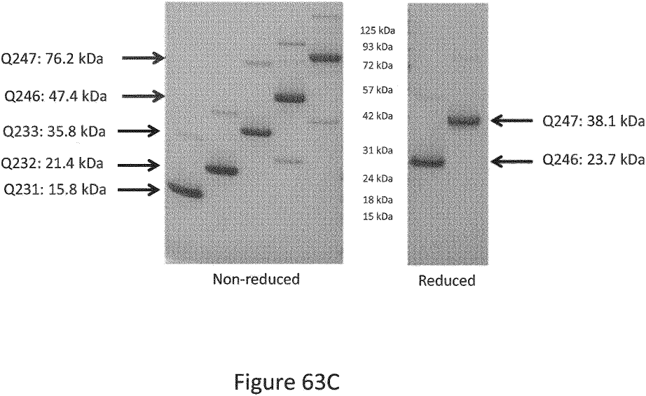

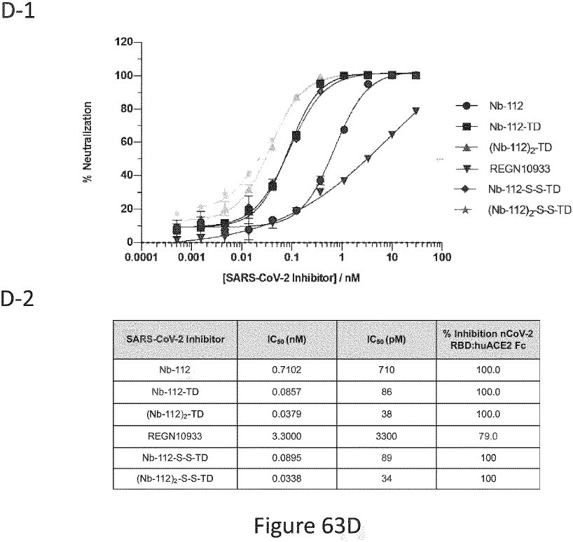

Following on from Example 33 where tetravalent and octavalent versions of Nb-112 were generated, in this example a modified tetravalent and octavalent version of Nb-112 were designed and produced where an intact hinge region was included in the construct. The hinge region allows for the dimerization of the monomeric building blocks to form dimers upon which these dimers are further dimerized in an anti-parallel manner through the p53 tetramerization domain to form tetramers. A schematic structural arrangement of these new tetravalent (also called Q246 or Nb-112-S-S-TD) and octavalent (also called Q247 or [Nb-112]₂-S-S-TD) Quads can be seen in FIGS. 63A and B respectively and their polypeptide sequences are shown in Table 34. The presence of the disulfide bonds could provide enhanced stability and functional neutralization potency to these multivalent Quads formats and this would be advantageous particularly when formulating these nanobody Quads through nebulisation.

Expression vectors for producing Q246 and Q247 were transfected into Expi293F cells and the proteins were produced as described in Example 28. As with Q232 and Q233 described in Example 33, the intrinsic binding properties of the VH3 subfamily for Protein A resin was exploited to purify both Q246 and Q247. The protein yields after Protein A affinity purification was measured using Nanospec and it can be seen the tetravalent (Q246) and octavalent (Q247) versions of Nb-112 yielded excellent protein titers equivalent to 350 mg/L and 265 mg/L respectively. The protein yields were similar to the tetravalent and octavalent versions described in Example 33 indicating that the present of the hinge region did not hamper protein production. To analyse the produced protein, a small aliquot (2 ug/well) were separated out on denaturing SDS-PAGE under non-reduced conditions alongside Nb-112 proteins produced in Example 33. For Q246 and Q247, the protein was also separated out under reduced conditions to visualize the monomeric building block polypeptide (FIG. 63C). Protein polypeptides for Q246 and Q247 migrated according to their expected molecular weight under both non-reduced and reduced conditions.

To investigate the functionality of the two new tetravalent and octavalent Nb-112 Quads (Q246 and Q247), a competitive ELISA was performed for their ability to neutralize the interaction of SARS-CoV-2 spike RBD with the ACE2 receptor as described in Example 33 using the same conditions. As a comparison, Q231-Q233 was also tested alongside Q246 and Q247 including the benchmark anti-SARS-CoV-2 antibody REGN10933. The data was plotted as percentage neutralization over a range of anti-SARS-CoV-2 inhibitor concentration (FIG. 63D-1).

From the in vitro neutralization assay data, a nice dose-dependent inhibition of SARS-CoV-2 interaction with ACE2 can be seen where the new tetravalent and octavalent versions (Q246 and Q247 respectively) were found to be significantly more potent than the monovalent Nb-112 VHH as seen from the IC₅₀ values (FIG. 63D-2). Interesting in this assay setting, the tetravalent versions (Q232 and Q246) had similar neutralization potencies to each other despite their differences in their structure. This was also true for the octavalent versions (Q233 and Q247).

For the reasons discussed in Example 33 in terms of the high affinity of Nb-112 VHH and the limited assay window, the real differences in neutralization potences might not be possible to be differentiated in this in vitro artificial assay setting. A cell-based assay such as a pseudovirus neutralization assay or even better an in vivo animal model would be required to fully characterize and tease out potency differences between these multivalent Quad formats. However, on the whole a good correlation with increase binding domain valency with increase neutralization potency can be seen when comparing the neutralization potences between the tetravalent and octavalent versions of Nb-112 to the monovalent version confirming that these new multivalent formats are functionally active and are capable of neutralizing SARS-CoV-2 through blocking its interaction with ACE2 receptor.

### Example 35: Production & Characterisation of Nebulised Quads

Importantly, these Quad therapeutics may be delivered via inhalation to a human or animal subject. Inhalation has major advantages over other routes of administration and could be one of the most important potential uses for a therapeutic for SARS-CoV-2. For example, nebulisation may be performed using a commercially available human-use Aeroneb Solo™ system (https://www.aerogen.com/aerogen-solo-3/), interfaced with a nose mask. The Aeroneb Solo nebuliser can, thus, be used to produce ~ 3 micron particles of Quads for lung delivery. This is suitable for deep lung delivery.

In an experiment, we will test once daily administration to lambs of a nebulised Quad. We expect that this will result in concentrations of the Quad in lung epithelial lining fluid that are at least 10 times higher than the in vitro EC₅₀ of the Quad for the cognate virus (eg, SARS-CoV-2). We will assess whether there is any detectable infectious virus in lung epithelial lining fluid, with no or low detectable virus supporting that the proposed route of administration could reduce infectivity. After administration of nebulised Quad, we expect blood concentrations will be lower (eg, 1000 fold lower or less) than lung epithelial lining fluid concentrations, which is important because low blood concentrations likely reduce systemic toxicity and risk of host antibody formation. Alternative routes of administration of Quads include intravenous, intramuscular or subcutaneous administration

Using the commercially available Aerogen Solo™ vibrating mesh nebuliser, we will nebulise Pichia pastoris-expressed Quad into an in line custom bead condenser. Analysis by size-exclusion chromatography on a Superdex™ 75 column will be carried out to establish no evidence of degradation or aggregation relative to the pre-nebulisation sample. From this, one can conclude that the Quad is resilient to degradation or aggregation during nebulisation.

Fourt different Quads (FIGS. 62 & 63) as follows will be tested
Quad 1- a multimer that comprises 4 copies of a polypeptide that comprises the amino acid sequence of SEQ ID NOs: 289;
Quad 2- a multimer that comprises 4 copies of a polypeptide that comprises the amino acid sequence of SEQ ID NOs: 290;
Quad 3- a multimer that comprises 4 copies of a polypeptide that comprises the amino acid sequence of SEQ ID NOs: 291; and
Quad 4- a multimer that comprises 4 copies of a polypeptide that comprises the amino acid sequence of SEQ ID NOs: 292.

### Nebulisation Stability Assessment of Quads

Stability following nebulisation of Pichia pastoris expressed Quad (eg, a Quad comprising copies of NIH-CoVnb-112) will be performed using an Aerogen Solo High-Performance Vibrating Mesh™ nebuliser placed in line with a custom glass bead condenser. A plastic culture tube will be fitted with a glass-pore frit and filled with sterilized 5 mm borosilicate glass beads. A three-way stopcock will be positioned distal to the frit to prevent pressurization during nebulisation. A 2 mg/mL SEC polished Quad solution will be prepared in 0.9% normal saline to model potential patient delivery. The Quad will be nebulised and the resulting condensate incubated at 37° C. for 24 hr to mimic exposure to body temperature. The nebulised, 37° C. treated Quad will be then collected for stability assessments and protein concentration measurements by BCA assay. Equal masses of pre and post-nebulisation samples will be denatured in LDS sample buffer (Invitrogen) and run on a NuPAGE 12% Bis-Tris precast polyacrylamide gel with SeeBlue Plue 2™ protein standards. Additional pre-and post-nebulisation samples will be injected onto a Superdex 75 Increase 10/300 GL size exclusion column operating on an AKTA Pure 25 M™ system.#

### Stability of Quads in Human Plasma

To further assess the stability of the Quad (eg, a Quad comprising copies of NIH-CoVnb-112), we will perform incubation of the Quad in pooled normal human plasma and recombinant human albumin followed by affinity measurement to assess preservation of antigen (eg, SARS-CoV-2 RBD) binding potential.

A Quad (eg, a Quad comprising copies of NIH-CoVnb-112) expressed in Pichiapastoris will be diluted from a concentrated stock solution into apheresis derived pooled human plasma (#IPLA-N, Innovative Research) to a final concentration of 5 µM and incubated at 37° C. for either 24 hr or 48 hr with gentle rotation. An identical sample set will be prepared at 5 µM in a solution containing 35 mg/mL recombinant human albumin (#A9731, Sigma-Aldrich) and incubated at 37° C. for either 24 hr or 48 hr with gentle rotation. A no-incubation control for each the plasma and recombinant human albumin conditions will be prepared at 5 µM. The samples will be prepared in a manner providing all conditions complete at the same time. Quad binding to antigen (eg, SARS-CoV-2 RBD) following treatment in pooled human plasma at time zero, 24 hr, and 48 hr will, we expect, have negligible impact on binding. Similarly, Quad binding to antigen following treatment in recombinant human albumin at all time points will, we expect, have no apparent effect on binding. Such data will support the interpretation that the Quad is satisfactorily stable in the presence of plasma.

The samples will be diluted 1:10 with IxPBS to yield a final Quad concentration of 500 nM and Bio-layer Interferometry will be performed using immobilized biotinylated antigen (eg, SARS-CoV-2 S protein RBD) to determine retention of binding potential.

### Determining Melting Temperature and Refolding by Circular Dichroism

Circular Dichroism (CD) will be performed using a Jasco J-815 Spectropolarimeter™. For thermal stability measurements the Quad will be diluted to 10 µg/mL in deionized water and placed in a quartz cuvette with 1 cm path length and CD measured at an ultraviolet wavelength of 205 nm. Quad will be heated from 25° C. to 85° C. at a rate of 2.5° C./min while stirring and then cooled back to 25° C. at the same rate. We expect that measurements using circular dichroism (CD) during heating will reveal that the Quad structure resists unfolding at elevated temperature (eg, until 74° C.) and upon cooling most (eg, at least 50, 60, 70 or 80%) of the structure will return to the baseline CD value. These data will support an extremely stable, robust, high affinity format.

### Exemplary Nebulised Quad Compositions

Using Aerogen Solo™ with an 8 stage cascade impactor running at a continuous flow rate of 28.3 LPM - see Table 37.

### Example 36: Quad PK and Efficacy Model

Quads (separately, Quads 1-4) will be produced using a HEK203, CHO or Pichia pastoris X-33 expression system. Formulation buffer will contain NaCl as osmolality agent and phosphate as buffer component.

### Drug Administration

An Aeroneb Solo™ System (Aerogen Ltd, Galway, Ireland), containing the Aeroneb Solo mesh nebulizer and the Aeroneb Pro-X™ controller will be used in accordance with the instruction manual as provided by the manufacturer. The estimated particle sizes obtained with these meshes will be around 3 to 3.5 µm (MMAD, Median mass aerodynamic diameter). The assembly and operation of the Aeroneb Solo System will be performed according to the nebulizer instruction manual. The nebulizer and the T-piece will be inserted into the breathing circuit. Air will be supplied to the system at an airflow speed of 2 L/min using a compressed air canister that is attached directly to the nebulizer T-piece.

Testing will be performed in lambs. Prior to dosing, the nebulizer reservoir will be filled with following volumes of different concentrations of the Quad formulation: either 4 mL (3 mg/kg target inhaled dose), 1.3 mL (1 mg/kg target inhaled dose) 0.4 mL (0.3 mg/kg target inhaled dose), 0.2 mL (0.08 mg/kg target inhaled dose) or 0.1 mL (0.04 mg/kg target inhaled dose). A cone mask (Cat # 05305, A.M. Bickford, Inc, US) will be attached to the nebulizer T-piece and placed over each lamb’s nose, mandible and maxilla. The nebulizer will be turned on at a constant nebulization mode and the cone mask firmly held in place during the duration of the nebulization. Once the dose has been nebulized (i.e., when the nebulizer reservoir is empty), the face mask will be removed and the nebulizer switched off. The lamb will then be returned to its cage and general health (alertness, responsiveness, ability to stand and move) will be monitored for 10 minutes.

### Pharmacokinetics of Quads in Neonatal Lambs

Four independent studies will be performed in SARS-CoV-2-infected neonatal lambs. In all of these studies, blood samples will be taken at selected time points followingthe first dose and all the subsequent doses for pharmacokinetic (PK) purposes. On Day 6, bronchoalveolar lavage fluid (BALF) sampling will be performed post-mortem for PK analysis in the lung compartment. Once daily administration of Quad via inhalation for 5 or 3 consecutive days is expected to result in high concentrations of Quad in lung epithelial lining fluid (ELF). A dose-dependent increase in ELF concentrations is expected to be seen on day 6.

### Effect of Daily Quad Administration to Neonatal Lambs When Started on Day 1 Post-infection

To assess the therapeutic efficacy of Quad when administered by inhalation, thirteen lambs (twelve lambs for analysis) will be inoculated with SARS-CoV-2 virus on day 0. The day after infection (day 1), the lambs will be randomized to either the placebo group or to Quad dose groups (0.3, 3 or 1 mg/kg) and treated daily by inhalation for 5 consecutive days. Lambs will undergo daily physical examinations and body weights, heart rates, rectal temperatures, respiratory distress and viral infection-related symptoms will be recorded. On day 6, the animals will be euthanized and lung lavage samples and lung tissues will be obtained for analysis of viral load in lung, histopathology and immunohistochemical analysis. Viral inoculation by inhalation will be confirmed for robust infection of all the analyzed lambs by reverse transcription quantitative polymerase chain reaction (RT-qPCR) performed on BALF and lung tissue. In the placebo-treated lambs, we expect gross and microscopic lung lesions induced by viral infection. We expect that treatment of lambs with Quad at all three doses will result in significant reductions in viral RNA copy numbers (eg, that range from 1.4 to 1.8 Log10 viral RNA copies/mL in BALF and between 0.8 to 1.9 Log10 viral RNA copies/mg in lung tissue). We expect that treated lambs will have much lower or undetectable infectious virus.

### Example 37: Anti-SARS-CoV-2 Quads to Combat Viral Mutation & Evolution

Since SARS-CoV-2 first emerged, recurrent mutations in spike have occurred during both human-to-human transmission (and spillover/spillback events between humans and animals. Among mutations associated with enhancement of human-to-human transmission, N501Y occurred in three distinct emerging human variants: B.1.1.7 lineage (or 20IB/501Y.V1), B.1.351 lineage (or 20H/501Y.V2), and P.1 lineage (or 20J/501Y.V3) that were originally identified in the United Kingdom, South Africa and Japan/Brazil, respectively. The key involvement of residue 501 in ACE2 binding may contribute to increased prevalence of mutations at this site in multiple distinct SARS-CoV-2 strains. The B.1.351 and P.1 lineages also carry the RBM mutations E484K and K417N/T. Meanwhile, the N-terminal domain (NTD) deletion AHV69-70 arose in the mink-associated Cluster V strain and in B.1.1.7. The B.1.1.7 also has ΔY144, and B.1.351 has a ΔLLA242-244 deletion. Other mutations that may facilitate immune escape (e.g., A222V, N439K and S477N) are frequently observed in patient samples. Three mutations in the receptor-binding motif (RBM) (Y453F, F486L and N501T), associated with cross-species transmission between minks and humans, emerged independently in distinct clusters, suggesting they could be vital points for new host adaptation. Additional selection pressures, not directly related to receptor adaptation may also exist, as evidenced by mutations outside the RBM in human-animal transmission (e.g., the N-terminal domain deletion AHV69-70, G261D and RBD point mutations V367F).

Mutations in the spike protein can have various impacts on antibody binding footprints and affinity, ranging from no effect to substantial impairment of recognition and binding. Amino acid substitutions or deletions that appeared once in spike might appear elsewhere, new variants having a different assortment of mutations may emerge, and current variants may acquire new single mutations. Thus, work was carried out to dissect the effects of key mutations individually to understand they affect binding molecule (antibody or Quad) activities. First, the binding affinity for full-length G614 HexaPro spike ectodomain and to the monomeric receptor-binding domain (RBD) was determined. Using high-throughput surface plasmon resonance analysis, those binding molecules that react with the RBD were sorted into different “communities”. Communities were defined by shared competition profiles in a matrix, in which each antibody was evaluated for its ability to either pair with or compete with other antibodies for binding to spike. Some additional communities of binding moleucles were identified against the NTD (N-terminal domain) by mapping of antigenic sites using electron microscopy. In total, 14 different communities that react with the spike S1 domain were identified. The antigenic landscape of spike determined by these 14 communities can be divided into binding footprints that: (a) overlap with the RBM (receptor-binding motif), (b) approach the RBD from the outer edge, (c) involve the inner face of the RBD and are accessible only in the “up” state, or (d) include the NTD.

To understand if certain binding molecule communities are more susceptible to particular emerging spike mutations than others, single-cycle VSV-based pseudoparticles bearing point mutations found in human and mink variants were generated and the susceptibility of each mutation to neutralization by the molecules was assessed. Notably, a Quad multimer comprising copies of variable domain QB-GB, which binds outside of the core RBM, was found to be resistant to all S1 mutations analyzed and retained neutralization comparable to the parent virus. This Quad was found to bind to the inner face of the RBD. Strong binding was observed (IC50 of 0.0017 µg/mL). Near 100% ACE2 blocking was surprisingly observed (99.84%), and yet the multimer does not contact core RBM residues. This Quad multimer and other multimers binding spike protein in the same region, therefore, may advantageously be more resistant to receptor-driven selection pressure.

### References

Ali, S.A. et al., 1999. Transferrin Trojan Horses as a Rational Approach for the Biological Delivery of Therapeutic Peptide Domains. Journal of Biological Chemistry, 274(34), pp.24066-24073.
Barbas et al. (1992) supra
Bentley, G.A. & Mariuzza, R.A., 1996. THE STRUCTURE OF THE T CELL ANTIGEN RECEPTOR. Annual Review of Immunology, 14(1), pp.563-590.
Binz, H.K. et al., 2003. Designing repeat proteins: well-expressed, soluble and stable proteins from combinatorial libraries of consensus ankyrin repeat proteins. Journal of molecular biology, 332(2), pp.489-503.
Bixby, K.A. et al., 1999. Zn2+-binding and molecular determinants of tetramerisation in voltage-gated K+ channels. Nature structural biology, 6(1), pp.38-43.
Borghouts, C., Kunz, C. & Groner, B., 2005. Peptide aptamers: recent developments for cancer therapy. Expert Opinion on Biological Therapy, 5(6), pp.783-797.
Breitling, F. et al., 1991. A surface expression vector for antibody screening. Gene, 104(2), pp.147-53.
Burton, D.R. et al., 1991. A large array of human monoclonal antibodies to type 1 human immunodeficiency virus from combinatorial libraries of asymptomatic seropositive individuals. Proceedings of the National Academy of Sciences of the United States of America, 88(22), pp.10134-7.
Chang, C.N., Landolfi, N.F. & Queen, C., 1991. Expression of antibody Fab domains on bacteriophage surfaces. Potential use for antibody selection. Journal of immunology (Baltimore, Md. : 1950), 147(10), pp.3610-4.
Chaudhary, V.K. et al., 1990. A rapid method of cloning functional variable-region antibody genes in Escherichia coli as single-chain immunotoxins. Proceedings of the National Academy of Sciences, 87(3), pp.1066-1070.
Chiswell, D.J. & McCafferty, J., 1992. Phage antibodies: will new “coliclonal” antibodies replace monoclonal antibodies? Trends in biotechnology, 10(3), pp.80-4.
Clackson, T. et al., 1991. Making antibody fragments using phage display libraries. Nature, 352(6336), pp.624-8.
Davis, M.M. & Bjorkman, P.J., 1988. T-cell antigen receptor genes and T-cell recognition. Nature, 334(6181), pp.395-402.
Ding, X.-F. et al., 2008. Characterization and Expression of a Human KCTD1 Gene Containing the BTB Domain, Which Mediates Transcriptional Repression and Homomeric Interactions. DNA and Cell Biology, 27(5), pp.257-265.
Fujiwara, Y. & Minor, D.L., 2008. X-ray Crystal Structure of a TRPM Assembly Domain Reveals an Antiparallel Four-stranded Coiled-coil. Journal of Molecular Biology, 383(4), pp.854-870.
Hawkins, R.E. & Winter, G., 1992. Cell selection strategies for making antibodies from variable gene libraries: trapping the memory pool. European journal of immunology, 22(3), pp.867-70.
Herrin, B.R. & Cooper, M.D., 2010. Alternative adaptive immunity in jawless vertebrates. Journal of immunology (Baltimore, Md. : 1950), 185(3), pp.1367-74.
Hoogenboom, H.R. et al., 1991. Multi-subunit proteins on the surface of filamentous phage: methodologies for displaying antibody (Fab) heavy and light chains. Nucleic acids research, 19(15), pp.4133-7.
Hosse, R.J., Rothe, A. & Power, B.E., 2006. A new generation of protein display scaffolds for molecular recognition. Protein science : a publication of the Protein Society, 15(1), pp.14-27.
Irving, R.A. et al., 2001. Ribosome display and affinity maturation: from antibodies to single V-domains and steps towards cancer therapeutics. Journal of immunological methods, 248(1-2), pp.31-45.
Jeffrey, P.D., Gorina, S. & Pavletich, N.P., 1995. Crystal structure of the tetramerisation domain of the p53 tumor suppressor at 1.7 angstroms. Science (New York, N.Y.), 267(5203), pp.1498-502.
Kang, A.S. et al., 1991. Linkage of recognition and replication functions by assembling combinatorial antibody Fab libraries along phage surfaces. Proceedings of the National Academy of Sciences, 88(10), pp.4363-4366.
Kohl, A. et al., 2003. Designed to be stable: Crystal structure of a consensus ankyrin repeat protein. Proceedings of the National Academy of Sciences, 100(4), pp.1700-1705.
Lerner, R.A. et al., 1992. Antibodies without immunization. Science (New York, N.Y.), 258(5086), pp.1313-4.
Liu, Y. et al., 2006. The tetramer structure of the Nervy homology two domain, NHR2, is critical for AML1/ETO’s activity. Cancer Cell, 9(4), pp.249-260.
Lowman, H.B. et al., 1991. Selecting high-affinity binding proteins by monovalent phage display. Biochemistry, 30(45), pp.10832-8.
. Marks et al. (1991) supra
Marks, J.D. et al., 1991. By-passing immunization. Human antibodies from V-gene libraries displayed on phage. Journal of molecular biology, 222(3), pp.581-97.
Marks, J.D. et al., 1992. Molecular evolution of proteins on filamentous phage. Mimicking the strategy of the immune system. The Journal of biological chemistry, 267(23), pp.16007-10.
McCafferty et al. (1990) supra
McCafferty, J. et al., 1990. Phage antibodies: filamentous phage displaying antibody variable domains. Nature, 348(6301), pp.552-4.
Moysey, R., Vuidepot, A.-L. & Boulter, J.M., 2004. Amplification and one-step expression cloning of human T cell receptor genes. Analytical biochemistry, 326(2), pp.284-6.
Panowski, S. et al., 2014. Site-specific antibody drug conjugates for cancer therapy. mAbs, 6(1), pp.34-45.
Parker, M.H. et al., 2005. Antibody mimics based on human fibronectin type three domain engineered for thermostability and high-affinity binding to vascular endothelial growth factor receptor two. Protein engineering, design & selection : PEDS, 18(9), pp.435-44.
Perez, H.L. et al., 2014. Antibody-drug conjugates: Current status and future directions. Drug Discovery Today, 19(7), pp.869-881.
Reddy Chichili, V.P., Kumar, V. & Sivaraman, J., 2013. Linkers in the structural biology of protein-protein interactions. Protein science : a publication of the Protein Society, 22(2), pp.153-67.
Shao, C.-Y., Secombes, C.J. & Porter, A.J., 2007. Rapid isolation of IgNAR variable single-domain antibody fragments from a shark synthetic library. Molecular immunology, 44(4), pp.656-65.
Silverman, J. et al., 2005. Multivalent avimer proteins evolved by exon shuffling of a family of human receptor domains. Nature Biotechnology, 23(12), pp.1556-1561.
Skerra, A., 2000. Lipocalins as a scaffold. Biochimica et Biophysica Acta (BBA) - Protein Structure and Molecular Enzymology, 1482(1-2), pp.337-350.
Thie, H. et al., 2009. Multimerization domains for antibody phage display and antibody production. New biotechnology, 26(6), pp.314-21.
Tonegawa, S., 1988. Somatic generation of immune diversity. Bioscience reports, 8(1), pp.3-26.
Walchli, S. et al., 2011. A Practical Approach to T-Cell Receptor Cloning and Expression M. M. Rodrigues, ed. PLoS ONE, 6(11), p.e27930.
Wikman, M. et al., 2004. Selection and characterization of HER2/neu-binding affibody ligands. Protein Engineering Design and Selection, 17(5), pp.455-462.
Young, P.A., Morrison, S.L. & Timmerman, J.M., 2014. Antibody-cytokine fusion proteins for treatment of cancer: Engineering cytokines for improved efficacy and safety. Seminars in Oncology, 41(5), pp.623-636.
Zahnd, C. et al., 2007. A designed ankyrin repeat protein evolved to picomolar affinity to Her2. Journal of molecular biology, 369(4), pp.1015-28.
Zhang, J. et al., 2009. Transient expression and purification of chimeric heavy chain antibodies. Protein Expression and Purification, 65(1), pp.77-82.
Expert Opinion on Investigational Drugs 16(6) 909-917 (June 2007)
Chapter 7 - Non- Antibody Scaffolds from Handbook of Therapeutic Antibodies (2007, edited by Stefan Dubel)
Adams, G.P. et al., 2006. Avidity-mediated enhancement of in vivo tumor targeting by single-chain Fv dimers. Clinical Cancer Research, 12(5), pp.1599-1605.
Alam, K. et al., 2018. A novel synthetic trivalent single chain variable fragment ( tri-scFv ) construction platform based on the SpyTag / SpyCatcher protein ligase system., pp.1-8.
Boruah, B.M. et al., 2013. Single Domain Antibody Multimers Confer Protection against Rabies Infection. PLoS ONE, 8(8).
Bru nker, P. et al., 2016. RG7386, a Novel Tetravalent FAP-DR5 Antibody, Effectively Triggers FAP-Dependent, Avidity-Driven DR5 Hyperclustering and Tumor Cell Apoptosis. Molecular Cancer Therapeutics, 15(5), pp.946-957. Available at: http://mct.aacrjournals.org/cgi/doi/10.1158/1535-7163.MCT-15-0647.
Li, L. et al., 2018. Amplification of CD20 Cross-Linking in Rituximab-Resistant B-Lymphoma Cells Enhances Apoptosis Induction by Drug-Free Macromolecular Therapeutics. ACS Nano, 12(4), pp.3658-3670.
Mayes, P.A., Hance, K.W. & Hoos, A., 2018. The promise and challenges of immune agonist antibody development in cancer. Nature Reviews Drug Discovery, 17(7), pp.509-527.
Rudnick, S.I. & Adams, G.P., 2009. Affinity and Avidity in Antibody-Based Tumor Targeting. Cancer Biotherapy & Radiopharmaceuticals, 24(2), pp.155-161. Available at: http://www.liebertonline.com/doi/abs/10.1089/cbr.2009.0627.
Shen, C. et al., 2019. An IgM antibody targeting the receptor binding site of influenza B blocks viral infection with great breadth and potency. Theranostics, 9(1), pp.210-231. Available at: http://www.thno.org/v09p0210.htm.
Slaga, D. et al., 2018. Avidity-based binding to HER2 results in selective killing of HER2-overexpressing cells by anti-HER2/CD3. Science Translational Medicine, 10(463).
Wu, A.M. et al., 2001. Multimerization of a chimeric anti-CD20 single-chain Fv-Fc fusion protein is mediated through variable domain exchange. Protein engineering, 14(12), pp.1025-1033.
Ying, T. et al., 2017. Fc engineering for Developing Therapeutic Bispecific Antibodies and Novel Scaffolds. Article, 8(January), p.1. Available at: www.frontiersin.org.Ying, T. et al., 2012. Soluble monomeric IgG1Fc. Journal of Biological Chemistry, 287(23), pp.19399-19408.
Shi, R et al. A human neutralizing antibody targets the receptor-binding site of SARS-CoV-2. Nature, Vol 584; 6 Aug. 2020.
Hansen, J et al. Studies in humanized mice and convalescent humans yield a SARS-CoV-2 antibody cocktail. Science 15 Jun. 2020.
T.J. Esparza, N.P. Martin, G.P. Anderson, E.R. Goldman and D.L. Brody, High affinity nanobodies block SARS-CoV-2 spike receptor binding domain interaction with human angiotensin converting enzyme, Sci. Rep. 10 (2020), pp. 1-13.
T.A. Seldon, K.E. Hughes, D.J. Munster, D.Y. Chin and M.L. Jones, Improved protein-A separation oƒ VH3ƒab from FC aƒter papain digestion oƒantibodies, J. Biomol. Tech. 22 (2012), pp. 50-52.

TABLE 1

p53 Sequences

| SEQ ID NO: | | AMINO ACID SEQUENCE | NOTES |
|---|---|---|---|
| 1 | Human p53 isoform 1 | MEEPQSDPSVEPPLSQETFSDLWKLLPENNVLSPLPSQAMDDLMLSPDDI EQWFTEDPGPDEAPRMPEAAPPVAPAPAAPTPAAPAPAPSWPLSSSVPSQ KTYQGSYGFRLGFLHSGTAKSVTCTYSPALNKMFCQLAKTCPVQLWVDST PPPGTRVRAMAIYKQSQHMTEVVRRCPHHERCSDSDGLAPPQHLIRVEGN LRVEYLDDRNTFRHSVVVPYEPPEVGSDCTTIHYNYMCNSSCMGGMNRRP ILTIITLEDSSGNLLGRNSFEVRVCACPGRDRRTEEENLRKKGEPHHELP PGSTKRALPNNTSSSPQPKKKPLDGEYFTLQIRGRERFEMFRELNEALEL KDAQAG KFPGGSRAHSSHLKSKKGQSTSRHKKLMFKTEGPDSD | Also known as: p53, p53alpha |
| | | | This isoform is denoted as the ‘canonical’ sequence. |
| | | | Tetramerisation sequence in underline bold (amino acid position numbers 325 to 356) |
| 2 | Human p53 isoform 2 | MEEPQSDPSVEPPLSQETFSDLWKLLPENNVLSPLPSQAMDDLMLSPDDI EQWFTEDPGPDEAPRMPEAAPPVAPAPAAPTPAAPAPAPSWPLSSSVPSQ KTYQGSYGFRLGFLHSGTAKSVTCTYSPALNKMFCQLAKTCPVQLWVDST PPPGTRVRAMAIYKQSQHMTEVVRRCPHHERCSDSDGLAPPQHLIRVEGN LRVEYLDDRNTFRHSVVVPYEPPEVGSDCTTIHYNYMCNS | Also known as: I9RET, p53beta The sequence of this isoform differs from the canonical sequence (isoform 1) as |
| | | SCMGGMNRRPILTIITLEDSSGNLLGRNSFEVRVCACPGRDRRTEEENLR KKGEPHHELPPGSTKRALPNNTSSSPQPKKKPLDGEYFTLQDQTSFQKEN C | follows: |
| | | | 332-341: IRGRERFEMF → DQTSFQKENC |
| | | | 342-393: Missing. |
| 3 | Human p53 isoform 3 | MEEPQSDPSVEPPLSQETFSDLWKLLPENNVLSPLPSQAMDDLMLSPDDI EQWFTEDPGPDEAPRMPEAAPPVAPAPAAPTPAAPAPAPSWPLSSSVPSQ KTYQGSYGFRLGFLHSGTAKSVTCTYSPALNKMFCQLAKTCPVQLWVDST PPPGTRVRAMAIYKQSQHMTEVVRRCPHHERCSDSDGLAPPQHLIRVEGN LRVEYLDDRNTFRHSVVVPYEPPEVGSDCTTIHYNYMCNSSCMGGMNRRP ILTIITLEDSSGNLLGRNSFEVRVCACPGRDRRTEEENLRKKGEPHHELP PGSTKRALPNNTSSSPQPKKKPLDGEYFTLQMLLDLRWCYFLINSS | Also known as: p53gamma |
| | | | The sequence of this isoform differs from the canonical sequence as follows: 332-346: IRGRERFEMFRELNE → MLLDLRWCYFLINSS |
| | | | 347-393: Missing |
| 4 | Human p53 | MDDLMLSPDDIEQWFTEDPGPDEAPRMPEAAPPVAPAPAAPTPAAPAPAP SWPLSSSVPSQKTYQGSYGFRLGFLHSGTAKSVTCTYSPALNKMFCQLAK TCPVQLWVDSTPPPGTRVRAMAIYKQSQHMTEVVRRCPHHERCSDSDGLA PPQHLIRVEGNLRVEYLDDRNTFRHSVVVP | Also known as: Del40-p53, Del40-p53alpha, p47 |
| | isoform 4 | YEPPEVGSDCTTIHYNYMCNSSCMGGMNRRPILTIITLEDSSGNLLGRNS FEVRVCACPGRDRRTEEENLRKKGEPHHELPPGSTKRALPNNTSSSPQPKK KPLDGEYFTLQIRGRERFEMFRELNEALELKDAQAGKEPGGSRAHSSHLK SKKGQSTSRHKKLMFKTEGPDSD | The sequence of this isoform differs from the canonical sequence as follows: |
| | | | 1-39: Missing. |
| 5 | Human p53 isoform 5 | MDDLMLSPDDIEQWFTEDPGPDEAPRMPEAAPPVAPAPAAPTPAAPAPAP SWPLSSSVPSQKTYQGSYGFRLGFLHSGTAKSVTCTYSPALNKMFCQLAK TCPVQLWVDSTPPPGTRVRAMAIYKQSQHMTEVVRRCPHHERCSDSDGLA PPQHLIRVEGNLRVEYLDDRNTFRHSVVVPYEPPEVGSDCTTIHYNYMCN SSCMGGMNRRPILTIITLEDSSGNLLGRNSFEVRVCACPGRDRRTEEENLR KKGEPHHELPPGSTKRALPNNTSSSPQPKKKPLDGEYFTLQDQTSFQKEN C | Also known as: Del40-p53beta. The sequence of this isoform differs from the canonical sequence as follows: |
| | | | 1-39: Missing. |
| | | | 332-341: IRGRERFEMF → DQTSFQKENC |
| | | | 342-393: Missing. |
| 6 | Human p53 isoform 6 | MDDLMLSPDDIEQWFTEDPGPDEAPRMPEAAPPVAPAPAAPTPAAPAPAP SWPLSSSVPSQKTYQGSYGFRLGFLHSGTAKSVTCTYSPALNKMFCQLAK TCPVQLWVDSTPPPGTRVRAMAIYKQSQHMTEVVRRCPHHERCSDSDGLA PPQHLIRVEGNLRVEYLDDRNTFRHSVVVPYEPPEVGSDCTTIHYNYMCN SSCMGGMNRRPILTIITLEDSSGNLLGRNSFEVRVCACPGRDRRTEEENLR KKGEPHHELPPGSTKRALPNNTSSSPQPKKKPLDGEYFTLQMLLDLRWCYF LINSS | Also known as: Del40-p53gamma. The sequence of this isoform differs from the canonical sequence as follows: |
| | | | 1-39: Missing. |
| | | | 332-346: IRGRERFEMFRELNE → MLLDLRWCYFLINSS |
| | | | 347-393: Missing. |
| 7 | Human p53 isoform 7 | MFCQLAKTCPVQLWVDSTPPPGTRVRAMAIYKQSQHMTEVVRRCPHHERC SDSDGLAPPQHLIRVEGNLRVEYLDDRNTFRHSVVVPYEPPEVGSDCTTI HYNYMCNSSCMGGMNRRPILTIITLEDSSGNLLGRNSFEVRVCACPGRDR RTEEENLRKKGEPHHELPPGSTKRALPNNT | Also known as: Dell33-p53, Dell33-p53alpha. The sequence of this isoform differs from the canonical sequence as follows: |
| | | SSSPQPKKKPLDGEYFTLQIRGRERFEMFRELNEALELKDAQAG KFPGGS RAHSSHLKSKKGQSTSRHKKLMFKTEGPDSD | 1-132: Missing. |
| 8 | Human p53 isoform 8 | MFCQLAKTCPVQLWVDSTPPPGTRVRAMAIYKQSQHMTEVVRRCPHHERCS DSDGLAPPQHLIRVEGNLRVEYLDDRNTFRHSVVVPYEPPEVGSDCTTIH YNYMCNSSCMGGMNRRPILTIITLEDSSGNLLGRNSFEVRVCACPGRDRRT EEENLRKKGEPHHELPPGSTKRALPNNTSSSPQPKKKPLDGEYFTLQDQT SFQKENC | Also known as: Dell133-p53beta. The sequence of this isoform differs from the canonical sequence as follows: |
| | | | 1-132: Missing. |
| | | | 332-341: IRGRERFEMF → DQTSFQKENC |
| | | | 342-393: Missing. |
| 9 | Human p53 isoform 9 | MFCQLAKTCPVQLWVDSTPPPGTRVRAMAIYKQSQHMTEVVRRCPHHERCS DSDGLAPPQHLIRVEGNLRVEYLDDRNTFRHSVVVPYEPPEVGSDCTTIH YNYMCNSSCMGGMNRRPIL | Also known as: Del133-p53gamma. The sequence of this isoform differs from the |
| | | TIITLEDSSGNLLGRNSFEVRVCACPGRDRRTEEENLRKKGEPHHELPPG STKRALPNNTSSSPQPKKKPLDGEYFTLQMLLDLRWCYFLINSS | canonical sequence as follows: |
| | | | 1-132: Missing. |
| | | | 332-346: IRGRERFEMFRELNE → MLLDLRWCYFLINSS |
| | | | 347-393: Missing. |
| 10 | A human p53-TD | GEYFTLQIRGRERFEMFRELNEALELKDAQAG | |
| 11 | A human p63-TD | RSPDDELLYLPVRGRETYEMLLKIKESLELMQYLPQHTIETYRQQQQQQH | |
| 12 | A human p73-TD | KKRRHGDEDTYYLQVRGRENFEILMKLKESLELMELVPQPLV | |

TABLE 2

Example Human Proteins Comprising a Tetramerisation Domain

| Protein Number | Uniprot Entry | Entry name | Protein names | Gene names |
|---|---|---|---|---|
| 1 | P04637 | P53 HUMAN | Cellular tumor antigen p53 (Antigen NY-CO-13) (Phosphoprotein p53) (Tumor suppressor p53) | TP53 P53 |
| 2 | Q719H9 | KCTD1 HUMAN | BTB/POZ domain-containing protein KCTD1 (Potassium channel tetramerisation domain-containing protein 1) | KCTD1 C18orf5 |
| 3 | P51787 | KCNQ1 HUMAN | Potassium voltage-gated channel subfamily KQT member 1 (IKs producing slow voltage-gated potassium channel subunit alpha KvLQT1) (KQT-like 1) (Voltage-gated potassium channel subunit Kv7.1) | KCNQ1KCNA8 KCNA9 KVLQT1 |
| 4 | Q06455 | MTG8_HUMAN | Protein CBFA2T1 (Cyclin-D-related protein) (Eight twenty one protein) (Protein ETO) (Protein MTG8) (Zinc finger MYND domain-containing protein 2) | RUNX1T1 AML1T1 CBFA2T1 CDR ETO MTG8 ZMYND2 |
| 5 | Q9H3F6 | BACD3 HUMAN | BTB/POZ domain-containing adapter for CUL3-mediated RhoA degradation protein 3 (hBACURD3) (BTB/POZ domain-containing protein KCTD10) (Potassium channel tetramerisation domain-containing protein 10) | KCTD 10 ULR061 MSTP028 |
| 6 | Q12809 | KCNH2 HUMAN | Potassium voltage-gated channel subfamily H member 2 (Eag | KCNH2 ERG ERG1 |
| | | | homolog) (Ether-a-go-go-related gene potassium channel 1) (ERG-1) (Eag-related protein 1) (Ether-a-go-go-related protein 1) (H-ERG) (hERG-1) (hERG1) (Voltage-gated potassium channel subunit Kv11. 1) | HERG |
| 7 | Q96SI1 | KCD15 HUMAN | BTB/POZ domain-containing protein KCTD15 (Potassium channel tetramerisation domain-containing protein 15) | KCTD15 |
| 8 | P02766 | TTHY HUMAN | Transthyretin (ATTR) (Prealbumin) (TBPA) | TTR PALB |
| 9 | Q14681 | KCTD2 HUMAN | BTB/POZ domain-containing protein KCTD2 (Potassium channel tetramerisation domain-containing protein 2) | KCTD2 KIAA0176 |
| 10 | Q7Z5Y7 | KCD20 HUMAN | BTB/POZ domain-containing protein KCTD20 (Potassium channel tetramerisation domain containing 20) | KCTD20 C6orf69 |
| 11 | P50552 | VASP_HUMAN | Vasodilator-stimulated phosphoprotein (VASP) | VASP |
| 12 | Q68DU8 | KCD16 HUMAN | BTB/POZ domain-containing protein KCTD16 (Potassium channel tetramerisation domain-containing protein 16) | KCTD16 KIAA1317 |
| 13 | Q09470 | KCNA1 HUMAN | Potassium voltage-gated channel subfamily A member 1 (Voltage-gated K(+) channel HuKI) (Voltage-gated potassium channel HBK1) (Voltage-gated potassium channel subunit Kv1.1) | KCNA1 |
| 14 | P13501 | CCL5_HUMAN | C-C motif chemokine 5 (EoCP) (Eosinophil chemotactic cytokine) (SIS-delta) (Small-inducible cytokine A5) (T cell-specific protein P228) (TCP228) (T-cell-specific protein | CCL5 D17S136E SCYA5 |
| | | | RANTES) [Cleaved into: RANTES(3-68); RANTES(4-68)] | |
| 15 | P08069 | IGF1R HUMAN | Insulin-like growth factor 1 receptor (EC 2.7.10.1) (Insulin-like growth factor I receptor) (IGF-I receptor) (CD antigen CD221) [Cleaved into: Insulin-like growth factor 1 receptor alpha chain; Insulin-like growth factor 1 receptor beta chain] | IGF1R |
| 16 | Q14003 | KCNC3 HUMAN | Potassium voltage-gated channel subfamily C member 3 (KSHIIID) (Voltage-gated potassium channel subunit Kv3.3) | KCNC3 |
| 17 | 015350 | P73_HUMAN | Tumor protein p73 (p53-like transcription factor) (p53-related protein) | TP73 P73 |
| 18 | Q12791 | KCMA1 HUMAN | Calcium-activated potassium channel subunit alpha-1 (BK channel) (BKCA alpha) (Calcium-activated potassium channel, subfamily M subunit alpha-1) (K(VCA)alpha) (KCa1.1) (Maxi K channel) (MaxiK) (Slo-alpha) (S1o1) (Slowpoke homolog) (Slo homolog) (hSlo) | KCNMA 1 KCNMA SLO |
| 19 | P42261 | GRIA1 HUMAN | Glutamate receptor 1 (GluR-1) (AMPA-selective glutamate receptor 1) (GluR-A) (GluR-K1) (Glutamate receptor ionotropic, AMPA 1) (GluA1) | GRIA1 GLUH1 GLUR1 |
| 20 | P22303 | ACES HUMAN | Acetylcholinesterase (AChE) (EC 3.1.1.7) | ACHE |
| 21 | P04040 | CATA_HUMAN | Catalase (EC 1.11.1.6) | CAT |
| 22 | Q9H8Y8 | GORS2 HUMAN | Golgi reassembly-stacking protein 2 (GRS2) (Golgi phosphoprotein 6) (GOLPH6) (Golgi reassembly-stacking protein of 55 kDa) (GRASP55) (p59) | GORASP2 GOLPH6 |
| 23 | P34897 | GLYM_HUMAN | Serine hydroxymethyltransferase, mitochondrial (SHMT) (EC 2.1.2.1) (Glycine hydroxymethyltransferase) (Serine methylase) | SHMT2 |
| 24 | P13760 | 2B14_HUMAN | HLA class II histocompatibility antigen, DRB1-4 beta chain (MHC class II antigen DRB1*4) (DR-4) (DR4) | HLA-DRB1 |
| 25 | P04229 | 2B 1 1_HUMAN | HLA class II histocompatibility antigen, DRB1-1 beta chain (MHC class II antigen DRB1* 1) (DR-1) (DR1) | HLA-DRB1 |
| 26 | P35914 | HMGCL HUMAN | Hydroxymethylglutaryl-CoA lyase, mitochondrial (HL) (HMG-CoA lyase) (EC 4.1.3.4) (3-hydroxy-3-methylglutarate-CoA lyase) | HMGCL |
| 27 | Q29974 | 2B1G_HUMAN | HLA class II histocompatibility antigen, DRB1-16 beta chain (MHC class II antigen DRB1* 16) (DR-16) (DR16) | HLA-DRB1 |
| 28 | Q9TQE0 | 2B19_HUMAN | HLA class II histocompatibility antigen, DRB_1-9 beta chain (MHC class II antigen DRB1*9) (DR-9) (DR9) | HLA-DRB1 |
| 29 | Q9UDR5 | AASS_HUMAN | Alpha-aminoadipic semialdehyde synthase, mitochondrial (LKR/SDH) [Includes: Lysine ketoglutarate reductase (LKR) (LOR) (EC 1.5.1.8); Saccharopine dehydrogenase (SDH) (EC 1.5.1.9)] | AASS |
| 30 | P13761 | 2B17_HUMAN | HLA class II histocompatibility antigen, DRB1-7 beta chain (MHC class II antigen DRB1*7) (DR-7) (DR7) | HLA-DRB1 |
| 31 | P49450 | CENPA_HUMAN | Histone H3-like centromeric protein A (Centromere autoantigen A) (Centromere protein A) (CENP-A) | CENPA |
| 32 | Q9Y2W7 | CSEN_HUMAN | Calsenilin (A-type potassium channel modulatory protein 3) (DRE-antagonist modulator) (DREAM) (Kv channel-interacting protein 3) (KChIP3) | KCNIP3 CSEN DREAM KCHIP3 |
| 33 | Q16630 | CPSF6_HUMAN | Cleavage and polyadenylation specificity factor subunit 6 (Cleavage and polyadenylation specificity factor 68 kDa subunit) (CFIm68) (CPSF 68 kDa subunit) (Pre-mRNA cleavage factor Im 68 kDa subunit) (Protein HPBRII-4/7) | CPSF6 CFIM68 |
| 34 | 043809 | CPSF5_HUMAN | Cleavage and polyadenylation specificity factor subunit 5 (Cleavage and polyadenylation specificity factor 25 kDa subunit) (CFIm25) (CPSF 25 kDa subunit) (Nucleoside diphosphate-linked moiety X motif 21) (Nudix motif 21) (Pre-mRNA cleavage factor Im 25 kDa subunit) | NUDT21 CFIM25 CPSF25 CPSF5 |
| 35 | Q8N684 | CPSF7_HUMAN | Cleavage and polyadenylation specificity factor subunit 7 (Cleavage and polyadenylation specificity factor 59 kDa subunit) (CFIm59) (CPSF 59 kDa subunit) (Pre-mRNA cleavage factor Im 59 kDa subunit) | CPSF7 |
| 36 | Q14999 | CUL7_HUMAN | Cullin-7 (CUL-7) | CUL7 KIAA0076 |
| 37 | Q9UI08 | EVL_HUMAN | Ena/VASP-like protein (Ena/vasodilator-stimulated phosphoprotein-like) | EVL RNB6 |
| 38 | Q05193 | DYN1_HUMAN | Dynamin-1 (EC 3.6.5.5) | DNM1 DNM |
| 39 | Q8N8S7 | ENAH HUMAN | Protein enabled homolog | ENAH MENA |
| 40 | Q96PP8 | GBP5_HUMAN | Guanylate-binding protein 5 (EC 3.6.5.-) (GBP-TA antigen) (GTP-binding protein 5) (GBP-5) (Guanine nucleotide-binding protein 5) | GBP5 UNQ2427/PRO4987 |
| 41 | Q92947 | GCDH_HUMAN | Glutaryl-CoA dehydrogenase, mitochondrial (GCD) (EC 1.3.8.6) | GCDH |
| 42 | Q13614 | MTMR2 HUMAN | Myotubularin-related protein 2 (Phosphatidylinositol-3,5-bisphosphate 3-phosphatase) (EC 3.1.3.95) (Phosphatidylinositol-3-phosphate phosphatase) (EC 3.1.3.64) | MTMR2 KIAA1073 |
| 43 | Q99784 | NOE1_HUMAN | Noelin (Neuronal olfactomedin-related ER localized protein) (Olfactomedin-1) | OLFM1 NOE1 NOEL1 |
| 44 | P50542 | PEX5_HUMAN | Peroxisomal targeting signal 1 receptor (PTS 1 receptor) (PTS1R) (PTS1-BP) (Peroxin-5) (Peroxisomal C-terminal targeting signal import receptor) (Peroxisome receptor 1) | PEX5 PXR1 |
| 45 | P42262 | GRIA2_HUMAN | Glutamate receptor 2 (GluR-2) (AMPA-selective glutamate receptor 2) (GluR-B) (GluR-K2) (Glutamate receptor ionotropic, AMPA 2) (GluA2) | GRIA2 GLUR2 |
| 46 | P48058 | GRIA4_HUMAN | Glutamate receptor 4 (GluR-4) (GluR4) (AMPA-selective glutamate receptor 4) (GluR-D) (Glutamate receptor | GRIA4 GLUR4 |
| | | | ionotropic, AMPA 4) (GluA4) | |
| 47 | P42263 | GRIA3_HUMAN | Glutamate receptor 3 (GluR-3) (AMPA-selective glutamate receptor 3) (GluR-C) (GluR-K3) (Glutamate receptor ionotropic, AMPA 3) (GluA3) | GRIA3 GLUR3 GLURC |
| 48 | 060741 | HCN1_HUMAN | Potassium/sodium hyperpolarization-activated cyclic nucleotide-gated channel 1 (Brain cyclic nucleotide-gated channel 1) (BCNG-1) | HCN1 BCNG1 |
| 49 | Q9UL51 | HCN2_HUMAN | Potassium/sodium hyperpolarization-activated cyclic nucleotide-gated channel 2 (Brain cyclic nucleotide-gated channel 2) (BCNG-2) | HCN2 BCNG2 |
| 50 | Q9Y3Q4 | HCN4_HUMAN | Potassium/sodium hyperpolarization-activated cyclic nucleotide-gated channel 4 | HCN4 |
| 51 | P04035 | HMDH_HUMAN | 3-hydroxy-3-methylglutaryl-coenzyme A reductase (HMG-CoA reductase) (EC 1.1.1.34) | HMGCR |
| 52 | Q8NCD3 | HJURP_HUMAN | Holliday junction recognition protein (14-3-3-associated AKT substrate) (Fetal liver-expressing gene 1 protein) (Up-regulated in lung cancer 9) | HJURP FAKTS FLEG1 URLC9 |
| 53 | Q9NZV8 | KCND2_HUMAN | Potassium voltage-gated channel subfamily D member 2 (Voltage-gated potassium channel subunit Kv4.2) | KCND2 KIAA1044 |
| 54 | P48547 | KCNC1_HUMAN | Potassium voltage-gated channel subfamily C member 1 (NGK2) (Voltage-gated potassium channel subunit Kv3.1) | KCNC1 |
| | | | (Voltage-gated potassium channel subunit Kv4) | |
| 55 | Q96CX2 | KCD12_HUMAN | BTB/POZ domain-containing protein KCTD12 (Pfetin) (Predominantly fetal expressed T1 domain) | KCTD12 C13orf2 KIAA1778 PFET1 |
| 56 | P16389 | KCNA2_HUMAN | Potassium voltage-gated channel subfamily A member 2 (NGK1) (Voltage-gated K(+) channel HuKIV) (Voltage-gated potassium channel HBK5) (Voltage-gated potassium channel subunit Kv1.2) | KCNA2 |
| 57 | P56696 | KCNQ4_HUMAN | Potassium voltage-gated channel subfamily KQT member 4 (KQT-like 4) (Potassium channel subunit alpha KvLQT4) (Voltage-gated potassium channel subunit Kv7.4) | KCNQ4 |
| 58 | Q9NXV2 | KCTD5_HUMAN | BTB/POZ domain-containing protein KCTD5 | KCTD5 |
| 59 | Q14721 | KCNB1_HUMAN | Potassium voltage-gated channel subfamily B member 1 (Delayed rectifier potassium channel 1) (DRK1) (h-DRK1) (Voltage-gated potassium channel subunit Kv2.1) | KCNB1 |
| 60 | Q86WG5 | MTMRD_HUMAN | Myotubularin-related protein 13 (SET-binding factor 2) | SBF2 CMT4B2 KIAA1766 MTMRl3 |
| 61 | Q15070 | OXA1L_HUMAN | Mitochondrial inner membrane protein OXA1L (Hsa) (OXA1Hs) (Oxidase assembly 1-like protein) (OXA1-like protein) | OXA1L |
| 62 | P11498 | PYC_HUMAN | Pyruvate carboxylase, mitochondrial (EC 6.4.1.1) (Pyruvic | PC |
| | | | carboxylase) (PCB) | |
| 63 | P33764 | S10A3_HUMAN | Protein S100-A3 (Protein S-100E) (S100 calcium-binding protein A3) | S100A3 S100E |
| 64 | P58743 | S26A5_HUMAN | Prestin (Solute carrier family 26 member 5) | SLC26A5 PRES |
| 65 | Q9UIL1 | SCOC_HUMAN | Short coiled-coil protein | SCOCSCOCO HRIHFB2072 |
| 66 | P02549 | SPTA1_HUMAN | Spectrin alpha chain, erythrocytic 1 (Erythroid alpha-spectrin) | SPTA1 SPTA |
| 67 | Q96QT4 | TRPM7_HUMAN | Transient receptor potential cation channel subfamily M member 7 (EC 2.7.11.1) (Channel-kinase 1) (Long transient receptor potential channel 7) (LTrpC-7) (LTrpC7) | TRPM7 CHAK1 LTRPC7 |
| 68 | Q9HCF6 | TRPM3_HUMAN | Transient receptor potential cation channel subfamily M member 3 (Long transient receptor potential channel 3) (LTrpC-3) (LTrpC3) (Melastatin-2) (MLSN2) | TRPM3 KIAA1616 LTRPC3 |
| 69 | Q7Z4N2 | TRPM1_HUMAN | Transient receptor potential cation channel subfamily M member 1 (Long transient receptor potential channel 1) (LTrpC1) (Melastatin-1) | TRPM1 LTRPC1 MLSN MLSN1 |
| 70 | Q9NQA5 | TRPV5_HUMAN | Transient receptor potential cation channel subfamily V member 5 (TrpV5) (Calcium transport protein 2) (CaT2) (Epithelial calcium channel 1) (ECaC) (ECaC1) (Osm-9-like TRP channel 3) (OTRPC3) | TRPV5 ECAC1 |
| 71 | Q9BX84 | TRPM6_HUMAN | Transient receptor potential cation channel subfamily M member 6 (EC 2.7.11.1) (Channel kinase 2) (Melastatin-related TRP cation channel 6) | TRPM6 CHAK2 |
| 72 | P49638 | TTPA_HUMAN | Alpha-tocopherol transfer protein (Alpha-TTP) | TTPA TPP 1 |
| 73 | Q8NBZ7 | UXS1_HUMAN | UDP-glucuronic acid decarboxylase 1 (EC 4.1.1.35) (UDP-glucuronate decarboxylase 1) (UGD) (UXS-1) | UXS1 UNQ2538/PRO6079 |
| 74 | Q13426 | XRCC4_HUMAN | DNA repair protein XRCC4 (X-ray repair cross-complementing protein 4) | XRCC4 |
| 75 | A0A0A6YY98 | A0A0A6YY98_HUMAN | Transient receptor potential cation channel subfamily V member 5 | TRPV5 |
| 76 | H0YLN8 | H0YLN8_HUMAN | Transient receptor potential cation channel subfamily M member 7 (Transient receptor potential cation channel, subfamily M, member 7, isoform CRA_c) | TRPM7 hCG_39859 |
| 77 | A0A0C4DFW9 | A0A0C4DFW9_HUMAN | Cellular tumor antigen p53 | TP73 hCG_19088 |
| 78 | G5E9G1 | G5E9G1_HUMAN | Transient receptor potential cation channel subfamily M member 3 (Transient receptor potential cation channel, subfamily M, member 3, isoform CRA_a) | TRPM3 hCG_2042991 |
| 79 | H7BYP 1 | H7BYP1_HUMAN | Transient receptor potential cation channel subfamily M member 3 (Transient receptor potential cation channel, subfamily M, member 3, isoform CRA_c) | TRPM3 hCG_2042991 |
| 80 | A0A024R4C3 | A0A024R4C3_HUMAN | Tumor protein p73, isoform CRA_a | TP73 hCG_19088 |
| 81 | A0A0S2Z4N5 | A0A0S2Z4N5_HUMAN | Tumor protein p63 isoform 1 (Tumor protein p73-like, isoform CRA_a) (Fragment) | TP63 TP73L hCG_16028 |
| 82 | A0A024R5V1 | A0A024R5V1_HUMAN | Transient receptor potential cation channel, subfamily M, member 7, isoform CRA_a | TRPM7 hCG_39859 |
| 83 | X5D8S6 | X5D8S6_HUMAN | Adenylosuccinate lyase (ASL) (EC 4.3.2.2) (Adenylosuccinase) (Fragment) | ADSL hCG_40060 |
| 84 | C9D7D0 | C9D7D0_HUMAN | Cellular tumor antigen p53 | TP63 |
| 85 | K7PPA8 | K7PPA8_HUMAN | Cellular tumor antigen p53 | TP53 |
| 86 | A2A3F4 | A2A3F4_HUMAN | Transient receptor potential cation channel subfamily M member 3 | TRPM3 |
| 87 | Q2XSC7 | Q2XSC7_HUMAN | Cellular tumor antigen p53 | TP53 |
| 88 | A0A024R209 | A0A024R209_HUMAN | Transient receptor potential cation channel, subfamily M, member 1, isoform CRA_a | TRPM1 hCG_37570 |
| 89 | Q1MSX0 | Q1MSX0_HUMAN | Cellular tumor antigen p53 (Fragment) | TP53 |
| 90 | A0A0G2JN34 | A0A0G2JN34_HUMAN | Transient receptor potential cation channel subfamily M member 1 | TRPM1 |
| 91 | H6U5S3 | H6U5S3_HUMAN | Cellular tumor antigen p53 (Fragment) | |
| 92 | H2EHT1 | H2EHT1_HUMAN | Cellular tumor antigen p53 | TP53 |
| 93 | H6U5S2 | H6U5S2_HUMAN | Cellular tumor antigen p53 (Fragment) | |
| 94 | B4DNI2 | B4DNI2_HUMAN | Cellular tumor antigen p53 | |
| 95 | C9D7C9 | C9D7C9_HUMAN | Cellular tumor antigen p53 | TP63 |
| 96 | B6E4X6 | B6E4X6_HUMAN | Cellular tumor antigen p53 | |
| 97 | A0A087WZU8 | A0A087WZU8_HUMAN | Cellular tumor antigen p53 | TP53 |
| 98 | K7PPU4 | K7PPU4_HUMAN | Cellular tumor antigen p53 | TP53 |
| 99 | A0A0U1RQC9 | A0A0U1RQC9_HUMAN | Cellular tumor antigen p53 | TP53 |
| 100 | Q5U0E4 | Q5U0E4_HUMAN | Cellular tumor antigen p53 | |
| 101 | Q53GA5 | Q53GA5_HUMAN | Cellular tumor antigen p53 (Fragment) | |
| 102 | A2A3F7 | A2A3F7_HUMAN | Transient receptor potential cation channel subfamily M member 3 | TRPM3 |
| 103 | B4DMH2 | B4DMH2_HUMAN | Cellular tumor antigen p53 | |
| 104 | B7Z8X6 | B7Z8X6_HUMAN | Cellular tumor antigen p53 | |
| 105 | A0A141PNN3 | A0A141PNN3_HUMAN | Cellular tumor antigen p53 | TP63 |
| 106 | A0A141PNN4 | A0A141PNN4_HUMAN | Cellular tumor antigen p53 | TP63 |
| 107 | A0A087X1Q1 | A0A087X1Q1_HUMAN | Cellular tumor antigen p53 | TP53 |
| 108 | E5RMA8 | E5RMA8_HUMAN | Cellular tumor antigen p53 | TP53 |
| 109 | A0A0S2Z4N6 | A0A0S2Z4N6_HUMAN | Tumor protein p63 isoform 2 (Fragment) | TP63 |
| 110 | E9PBI7 | E9PBI7_HUMAN | Transient receptor potential cation channel subfamily M member 3 | TRPM3 |
| 111 | A0A0G2JMR4 | A0A0G2JMR4_HUMAN | Transient receptor potential cation channel subfamily M member 1 | TRPM1 |
| 112 | Q9H637 | Q9H637_HUMAN | cDNA: FLJ22628 fis, clone HSI06177 | |
| 113 | A0A0G2JPN6 | A0A0G2JPN6_HUMAN | Transient receptor potential cation channel subfamily M | TRPM1 |
| | | | member 1 | |
| 114 | A0A024R212 | A0A024R212_HUMAN | Transient receptor potential cation channel, subfamily M, member 1, isoform CRA_b | TRPM1 hCG_37570 |
| 115 | A0A0G2JMJ5 | A0A0G2JMJ5_HUMAN | Transient receptor potential cation channel subfamily M member 1 | TRPM1 |
| 116 | H0YM61 | H0YM61_HUMAN | Transient receptor potential cation channel subfamily M member 1 (Fragment) | TRPM1 |
| 117 | H0YKU7 | H0YKU7_HUMAN | Transient receptor potential cation channel subfamily M member 1 (Fragment) | TRPM1 |
| 118 | A0A0A0MTQ9 | A0A0A0MTQ9_HUMAN | Transient receptor potential cation channel subfamily M member 1 (Fragment) | TRPM1 |
| 119 | A2A3F3 | A2A3F3_HUMAN | Transient receptor potential cation channel subfamily M member 3 | TRPM3 |

The amino acid and nucleotide sequences of each of these proteins and the TD thereof is incorportated herein by reference for use in the present invention and for potential inclusion in one or more claims herein.

TABLE 3

DNA sequences encoding Quad polypeptides

| | | |
|---|---|---|
| SEQ ID NO: | POLYPEPTIDE | NUCLEOTIDE SEQUENCE |
| 13 | Quad 1 | ATGAGCCTCGGGCTCCTGTGCTGTGGGGCCTTTTCTCTCCTGTGGGCAGG TCCAGTGAATGCCGGTGTCACTCAGACCCCAAAATTCCAGGTCCTGAAGA CAGGACAGAGCATGACACTGCAGTGTGCCCAGGATATGAACCATGAATAC ATGTCCTGGTATCGACAAGACCCAGGCATGGGGCTGAGGCTGATTCATTA CTCAGTTGCCATCCAGACAACTGACCAAGGAGAAGTCCCCAATGGCTACA ATGTCTCCAGATCAACCATCGAGGATTTCCCGCTCAGGCTGCTGTCGGCT GCTCCCTCCCAGACATCTGTGTACTTCTGTGCCAGCAGTTACCTGGGGAA CACCGGGGAGCTGTTTTTTGGAGAAGGCTCTAGGCTGACCGTACTGGAGG ACCTGAAAAACGTGTTCCCACCCGAGGTCGCTGTGTTTGAGCCATCAGAA GCAGAGATCTCCCACACCCAAAAGGCCACACTGGTGTGCCTGGCCACAG GCTTCTACCCCGACCATGTGGAGCTGAGCTGGTGGGTGAATGGGAAGGAG GTGCACAGTGGGGTCAGCACAGACCCGCAGCCCCTCAAGGAGCAGCCCGC CCTCAATGACTCCAGATACGCTCTGAGCAGCCGCCTGAGGGTCTCGGCCA CCTTCTGGCAGGACCCCCGCAACCACTTCCGCTGTCAAGTCCAGTTCTAC GGGCTCTCGGAGAATGACGAGTGGACCCAGGATAGGGCCAAACCCGTCAC CCAGATCGTCAGCGCCGAGGCCTGG |
| | | GGTAGAGCAGACGCCAGCACCAAGGGCCCCTCTGTGTTCCCTCTGGCCCCT TCCAGCAAGTCCACCTCTGGCGGAACAGCCGCTCTGGGCTGCCTCGTGAAG GACTACTTCCCCGAGCCTGTGACCGTGTCCTGGAACTCTGGCGCTCTGAC CAGCGGAGTGCACACCTTCCCTGCTGTGCTGCAGTCCTCCGGCCTGTACT CCCTGTCCTCCGTCGTGACCGTGCCTTCCAGCTCTCTGGGCACCCAGACC TACATCTGCAACGTGAACCACAAGCCCTCCAACACCAAGGTGGACAAGAA GGTGCTAACAGACAGAGAATGGGCAGAAGAGTGGAAACATCTTGACCATCT GTTAAACTGCATAATGGACATGGTAGAAAAAACAAGGCGATCTCTCACCG TACTAAGGCGGTGTCAAGAAGCAGACCGGGAAGAATTGAATTACTGGATC CGGCGGTACAGTGACGCCGAG |
| 14 | Quad 2 | ATGAGCCTCGGGCTCCTGTGCTGTGGGGCCTTTTCTCTCCTGTGGGCAGG TCCAGTGAATGCCGGTGTCACTCAGACCCCAAAATTCCAGGTCCTGAAGA CAGGACAGAGCATGACACTGCAGTGTGCCCAGGATATGAACCATGAATAC ATGTCCTGGTATCGACAAGACCCAGGCATGGGGCTGAGGCTGATTCATTAC TCAGTTGCCATCCAGACAACTGACCAAGGAGAAGTCCCCAATGGCTACAA TGTCTCCAGATCAACCATCGAGGATTTCCCGCTCAGGCTGCTGTCGGCTGC TCCCTCCCAGACATCTGTGTACTTCTGTGCCAGCAGTTACCTGGGGAACACC GGGGAGCTGTTTTTTGGAGAAGGCTCTAGGCTGACCGTACTGGAGGACCTG AAAAACGTGTTCCCACCCGAGGTCGCTGTGTTTGAGCCATCAGAAGCAGAGA TCTCCCACACCCAAAAG |
| | | GCCACACTGGTGTGCCTGGCCACAGGCTTCTACCCCGACCATGTGGAGCTGAG CTGGTGGGTGAATGGGAAGGAGGTGCACAGTGGGGTCAGCACAGACCCGC AGCCCCTCAAGGAGCAGCCCGCCCTCAATGACTCCAGATACGCTCTGAGC AGCCGCCTGAGGGTCTCGGCCACCTTCTGGCAGGACCCCCGCAACCACTTCC GCTGTCAAGTCCAGTTCTACGGGCTCTCGGAGAATGACGAGTGGACCCAGG ATAGGGCCAAACCCGTCACCCAGATCGTCAGCGCCGAGGCCTGGGGTAGA GCAGACGCCAGCACCAAGGGCCCCTCTGTGTTCCCTCTGGCCCCTTCCAG CAAGTCCACCTCTGGCGGAACAGCCGCTCTGGGCTGCCTCGTGAAGGACTAC TTCCCCGAGCCTGTGACCGTGTCCTGGAACTCTGGCGCTCTGACCAGCGG AGTGCACACCTTCCCTGCTGTGCTGCAGTCCTCCGGCCTGTACTCCCTGT CCTCCGTCGTGACCGTGCCTTCCAGCTCTCTGGGCACCCAGACCTACATC TGCAACGTGAACCACAAGCCCTCCAACACCAAGGTGGACAAGAAGGTGGG AGGAGGTGGGAGCCTAACAGACAGAGAATGGGCAGAAGAGTGGAAACATCT TGACCATCTGTTAAACTGCATAATGGACATGGTAGAAAAAACAAGGCGATCT CTCACCGTACTAAGGCGGTGTCAAGAAGCAGACCGGGAAGAATTGAATTACT GGATCCGGCGGTACAGTGACGCCGAG |
| 15 | Quad 3 | ATGAGCCTCGGGCTCCTGTGCTGTGGGGCCTTTTCTCTCCTGTGGGCAGGT CCAGTGAATGCCGGTGTCACTCAGACCCCAAAATTCCAGGTCCTGAAGACAG GACAGAGCATG |
| | | ACACTGCAGTGTGCCCAGGATATGAACCATGAATACATGTCCTGGTATCGACA AGACCCAGGCATGGGGCTGAGGCTGATTCATTACTCAGTTGCCATCCAGA CAACTGACCAAGGAGAAGTCCCCAATGGCTACAATGTCTCCAGATCAACC ATCGAGGATTTCCCGCTCAGGCTGCTGTCGGCTGCTCCCTCCCAGACATCTGT GTACTTCTGTGCCAGCAGTTACCTGGGGAACACCGGGGAGCTGTTTTTTG GAGAAGGCTCTAGGCTGACCGTACTGGAGGACCTGAAAAACGTGTTCCCACC CGAGGTCGCTGTGTTTGAGCCATCAGAAGCAGAGATCTCCCACACCCAAAAGG CCACACTGGTGTGCCTGGCCACAGGCTTCTACCCCGACCATGTGGAGCTGAG CTGGTGGGTGAATGGGAAGGAGGTGCACAGTGGGGTCTGCACAGACCCGCA GCCCCTCAAGGAGCAGCCCGCCCTCAATGACTCCAGATACGCTCTGAGCAGC CGCCTGAGGGTCTCGGCCACCTTCTGGCAGGACCCCCGCAACCACTTCCGC TGTCAAGTCCAGTTCTACGGGCTCTCGGAGAATGACGAGTGGACCCAGGAT AGGGCCAAACCCGTCACCCAGATCGTCAGCGCCGAGGCCTGGGGTAGAGCAG ACGCCAGCACCAAGGGCCCCTCTGTGTTCCCTCTGGCCCCTTCCAGCAAGTCC ACCTCTGGCGGAACAGCCGCTCTGGGCTGCCTCGTGAAGGACTACTTCCCC GAGCCTGTGACCGTGTCCTGGAACTCTGGCGCTCTGACCAGCGGAGTGCA CACCTTCCCTGCTGTGCTGCAGTCCTCCGGCCTGTACTCCCTGTCCTCCG TCGTGACCGTGCCTTCCAGCTCTCTGGGCACCCAGACCTACATCTGCAAC GTGAACCACAAGCCCTCCAACACCAAGGTGGACAAG |
| | | AAGGTGCTAACAGACAGAGAATGGGCAGAAGAGTGGAAACATCTTGACCA TCTGTTAAACTGCATAATGGACATGGTAGAAAAAACAAGGCGATCTCTCA CCGTACTAAGGCGGTGTCAAGAAGCAGACCGGGAAGAATTGAATTACTGG ATCCGGCGGTACAGTGACGCCGAG |
| 16 | Quad 4 | ATGAGCCTCGGGCTCCTGTGCTGTGGGGCCTTTTCTCTCCTGTGGGCAGG TCCAGTGAATGCCGGTGTCACTCAGACCCCAAAATTCCAGGTCCTGAAGA CAGGACAGAGCATGACACTGCAGTGTGCCCAGGATATGAACCATGAATAC ATGTCCTGGTATCGACAAGACCCAGGCATGGGGCTGAGGCTGATTCATTA CTCAGTTGCCATCCAGACAACTGACCAAGGAGAAGTCCCCAATGGCTACAATGT CTCCAGATCAACCATCGAGGATTTCCCGCTCAGGCTGCTGTCGGCTGCTCC CTCCCAGACATCTGTGTACTTCTGTGCCAGCAGTTACCTGGGGAACACCGGG GAGCTGTTTTTTGGAGAAGGCTCTAGGCTGACCGTACTGGAGGACCTGAAAA ACGTGTTCCCACCCGAGGTCGCTGTGTTTGAGCCATCAGAAGCAGAGATCTC CCACACCCAAAAGGCCACACTGGTGTGCCTGGCCACAGGCTTCTACCCCG ACCATGTGGAGCTGAGCTGGTGGGTGAATGGGAAGGAGGTGCACAGTGGG GTCTGCACAGACCCGCAGCCCCTCAAGGAGCAGCCCGCCCTCAATGACTC CAGATACGCTCTGAGCAGCCGCCTGAGGGTCTCGGCCACCTTCTGGCAGG ACCCCCGCAACCACTTCCGCTGTCAAGTCCAGTTCTACGGGCTCTCGGAGA ATGACGAGTGGACCCAGGATAGGGCCAAACCCGTCACCCAGATCGTCAGC GCCGAGGCCTGG |
| | | GGTAGAGCAGACGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGGGAGGAGGTGGGAGCCTAACAGACAGAGAATGGGCAGAAGAGTGGAAACATCTT GACCATCTGTTAAACTGCATAATGGACATGGTAGAAAAAACAAGGCGATCTCTCACCGTA CTAAGGCGGTGTCAAGAAGCAGACCGGGAAGAATTGAATTACTGGATCCGGCGGTACAGT GACGCCGAG |
| 17 | Quad 5 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG GCCACACTGGTGTGCCTGGCCACAGGCTTCTACCCCGACCATGTGGAGCTGAGCTGGTGG GTGAATGGGAAGGAGGTGCACAGTGGGGTCTGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT GACGAGTGGACCCAGGATAGGGCCAAACCCGTCACCCAGATCGTCAGCGCCGAGGCCTGG GGTAGAGCAGACCTAACAGACAGAGAATGGGCAGAAGAGTGGAAACATCTTGACCATCTG TTAAACTGCATAATGGACATGGTAGAAAAAACAAGGCGATCTCTCACCGTACTAAGGCGG TGTCAAGAAGCAGACCGGGAAGAATTGAATTACTGGATCCGGCGGTACAGTGACGCCGAG |
| 18 | Quad 6 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG TTCCCACCCGAGGTCGCTGTGTTTGAGCCATCAGAAGCAGAGATCTCCCACACCCAAAAG GCCACACTGGTGTGCCTGGCCACAGGCTTCTACCCCGACCATGTGGAGCTGAGCTGGTGG GTGAATGGGAAGGAGGTGCACAGTGGGGTCTGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT GACGAGTGGACCCAGGATAGGGCCAAACCCGTCACCCAGATCGTCAGCGCCGAGGCCTGG GGTAGAGCAGACGGAGGAGGTGGGAGCCTAACAGACAGAGAATGGGCAGAAGAGTGGAAA CATCTTGACCATCTGTTAAACTGCATAATGGACATGGTAGAAAAAACAAGGCGATCTCTC ACCGTACTAAGGCGGTGTCAAGAAGCAGACCGGGAAGAATTGAATTACTGGATCCGGCGG TACAGTGACGCCGAG |
| 19 | Quad 7 | ATGGAGACCCTCTTGGGCCTGCTTATCCTTTGGCTGCAGCTGCAATGGGTGAGC AGCAAACAGGAGGTGACACAGATTCCTGCAGCTCTGAGTGTCCCAGAAGGAGAAAACTTG GTTCTCAACTGCAGTTTCACTGATAGCGCTATTTACAACCTCCAGTGGTTTAGGCAGGAC CCTGGGAAAGGTCTCACATCTCTGTTGCTTATTACACCTTGGCAGAGAGAGCAAACAAGT GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA TACATACCTACATTTGGAAGAGGAACCAGCCTTATTGTTCATCCGTATATCCAGAACCCT GACCCTGCCGTGTACCAGCTGAGAGACTCTAAATCCAGTGACAAGTCTGTCTGCCTATTC ACCGATTTTGATTCTCAAACAAATGTGTCACAAAGTAAGGATTCTGATGTGTATATCACA GACAAAACTGTGCTAGACATGAGGTCTATGGACTTCAAGAGCAACAGTGCTGTGGCCTGG AGCAACAAATCTGACTTTGCATGTGCAAACGCCTTCAACAACAGCATTATTCCAGAAGAC ACCTTCTTCCCCAGCCCAGAAAGTTCCACGGTGGCCGCTCCCTCCGTGTTCATCTTCCCA CCTTCCGACGAGCAGCTGAAGTCCGGCACCGCTTCTGTCGTGTGCCTGCTGAACAACTTC TACCCCCGCGAGGCCAAGGTGCAGTGGAAGGTGGACAACGCCCTGCAGTCCGGCAACTCC CAGGAATCCGTGACCGAGCAGGACTCCAAGGACAGCACCTACTCCCTGTCCTCCACCCTG ACCCTGTCCAAGGCCGACTACGAGAAGCACAAGGTGTACGCCTGCGAAGTGACCCACCAG GGCCTGTCTAGCCCCGTGACCAAGTCTTTCAACCGGGGCGAGTGT |
| 20 | Quad 8 | ATGGAGACCCTCTTGGGCCTGCTTATCCTTTGGCTGCAGCTGCAATGGGTGAGC AGCAAACAGGAGGTGACACAGATTCCTGCAGCTCTGAGTGTCCCAGAAGGAGAAAACTTG GTTCTCAACTGCAGTTTCACTGATAGCGCTATTTACAACCTCCAGTGGTTTAGGCAGGAC |
| | | CCTGGGAAAGGTCTCACATCTCTGTTGCTTATTACACCTTGGCAGAGAGAGCAAACAAGT GGAAGACTTAATGCCTCGCTGGATAAATCATCAGGACGTAGTACTTTATACATTGCAGCT TCTCAGCCTGGTGACTCAGCCACCTACCTCTGTGCTGTGAGGCCCCTGCTTGACGGAACA TACATACCTACATTTGGAAGAGGAACCAGCCTTATTGTTCATCCGTATATCCAGAACCCT GACCCTGCCGTGTACCAGCTGAGAGACTCTAAATCCAGTGACAAGTCTGTCTGCCTATTC ACCGATTTTGATTCTCAAACAAATGTGTCACAAAGTAAGGATTCTGATGTGTATATCACA GACAAATGTGTGCTAGACATGAGGTCTATGGACTTCAAGAGCAACAGTGCTGTGGCCTGG AGCAACAAATCTGACTTTGCATGTGCAAACGCCTTCAACAACAGCATTATTCCAGAAGAC ACCTTCTTCCCCAGCCCAGAAAGTTCCACGGTGGCCGCTCCCTCCGTGTTCATCTTCCCA CCTTCCGACGAGCAGCTGAAGTCCGGCACCGCTTCTGTCGTGTGCCTGCTGAACAACTTC TACCCCCGCGAGGCCAAGGTGCAGTGGAAGGTGGACAACGCCCTGCAGTCCGGCAACTCC CAGGAATCCGTGACCGAGCAGGACTCCAAGGACAGCACCTACTCCCTGTCCTCCACCCTG ACCCTGTCCAAGGCCGACTACGAGAAGCACAAGGTGTACGCCTGCGAAGTGACCCACCAG GGCCTGTCTAGCCCCGTGACCAAGTCTTTCAACCGGGGCGAGTGT |
| 21 | Quad 9 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAAAACTTG GTTCTCAACTGCAGTTTCACTGATAGCGCTATTTACAACCTCCAGTGGTTTAGGCAGGAC CCTGGGAAAGGTCTCACATCTCTGTTGCTTATTACACCTTGGCAGAGAGAGCAAACAAGT GGAAGACTTAATGCCTCGCTGGATAAATCATCAGGACGTAGTACTTTATACATTGCAGCT TCTCAGCCTGGTGACTCAGCCACCTACCTCTGTGCTGTGAGGCCCCTGCTTGACGGAACA TACATACCTACATTTGGAAGAGGAACCAGCCTTATTGTTCATCCGTATATCCAGAACCCT GACCCTGCCGTGTACCAGCTGAGAGACTCTAAATCCAGTGACAAGTCTGTCTGCCTATTC ACCGATTTTGATTCTCAAACAAATGTGTCACAAAGTAAGGATTCTGATGTGTATATCACA GACAAATGTGTGCTAGACATGAGGTCTATGGACTTCAAGAGCAACAGTGCTGTGGCCTGG AGCAACAAATCTGACTTTGCATGTGCAAACGCCTTCAACAACAGCATTATTCCAGAAGAC ACCTTCTTCCCCAGCCCAGAAAGTTCC |
| 22 | Quad 10 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC GGGGAGCTGTTTTTTGGAGAAGGCTCTAGGCTGACCGTACTGGAGGACCTGAAAAACGTG TTCCCACCCGAGGTCGCTGTGTTTGAGCCATCAGAAGCAGAGATCTCCCACACCCAAAAG GCCACACTGGTGTGCCTGGCCACAGGCTTCTACCCCGACCATGTGGAGCTGAGCTGGTGG GTGAATGGGAAGGAGGTGCACAGTGGGGTCAGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT GACGAGTGGACCCAGGATAGGGCCAAACCCGTCACCCAGATCGTCAGCGCCGAGGCCTGG GGTAGAGCAGACGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGCTAACAGACAGAGAATGGGCAGAAGAGTGGAAACATCTTGACCATCTGTTAAAC TGCATAATGGACATGGTAGAAAAAACAAGGCGATCTCTCACCGTACTAAGGCGGTGTCAA |
| | | GAAGCAGACCGGGAAGAATTGAATTACTGGATCCGGCGGTACAGTGACGCCGAGGCACCT ACTTCAAGTTCTACAAAGAAAACACAGCTACAACTGGAGCATTTACTGCTGGATTTACAG ATGATTTTGAATGGAATTAATAATTACAAGAATCCCAAACTCACCAGGATGCTCACATTT AAGTTTTACATGCCCAAGAAGGCCACAGAACTGAAACATCTTCAGTGTCTAGAAGAAGAA CTCAAACCTCTGGAGGAAGTGCTAAATTTAGCTCAAAGCAAAAACTTTCACTTAAGACCC AGGGACTTAATCAGCAATATCAACGTAATAGTTCTGGAACTAAAGGGATCTGAAACAACA TTCATGTGTGAATATGCTGATGAGACAGCAACCATTGTAGAATTTCTGAACAGATGGATT ACCTTTTGTCAAAGCATCATCTCAACACTGACT |
| 23 | Quad 11 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC GGGGAGCTGTTTTTTGGAGAAGGCTCTAGGCTGACCGTACTGGAGGACCTGAAAAACGTG TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG GTGAATGGGAAGGAGGTGCACAGTGGGGTCAGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT GACGAGTGGACCCAGGATAGGGCCAAACCCGTCACCCAGATCGTCAGCGCCGAGGCCTGG GGTAGAGCAGACGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGGGAGGAGGTGGGAGCCTAACAGACAGAGAATGGGCAGAAGAGTGGAAACATCTT GACCATCTGTTAAACTGCATAATGGACATGGTAGAAAAAACAAGGCGATCTCTCACCGTA CTAAGGCGGTGTCAAGAAGCAGACCGGGAAGAATTGAATTACTGGATCCGGCGGTACAGT GACGCCGAGGCACCTACTTCAAGTTCTACAAAGAAAACACAGCTACAACTGGAGCATTTA CTGCTGGATTTACAGATGATTTTGAATGGAATTAATAATTACAAGAATCCCAAACTCACC AGGATGCTCACATTTAAGTTTTACATGCCCAAGAAGGCCACAGAACTGAAACATCTTCAG |
| | | TGTCTAGAAGAAGAACTCAAACCTCTGGAGGAAGTGCTAAATTTAGCTCAAAGCAAAAAC TTTCACTTAAGACCCAGGGACTTAATCAGCAATATCAACGTAATAGTTCTGGAACTAAAG GGATCTGAAACAACATTCATGTGTGAATATGCTGATGAGACAGCAACCATTGTAGAATTT CTGAACAGATGGATTACCTTTTGTCAAAGCATCATCTCAACACTGACT |
| 24 | Quad 12 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC GGGGAGCTGTTTTTTGGAGAAGGCTCTAGGCTGACCGTACTGGAGGACCTGAAAAACGTG TTCCCACCCGAGGTCGCTGTGTTTGAGCCATCAGAAGCAGAGATCTCCCACACCCAAAAG GCCACACTGGTGTGCCTGGCCACAGGCTTCTACCCCGACCATGTGGAGCTGAGCTGGTGG GTGAATGGGAAGGAGGTGCACAGTGGGGTCAGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG GGTAGAGCAGACGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGGAACCCAAGTCCTGCGACAAGACCCACACCTGTCCCCCTTGTCCTCTAACAGAC AGAGAATGGGCAGAAGAGTGGAAACATCTTGACCATCTGTTAAACTGCATAATGGACATG GTAGAAAAAACAAGGCGATCTCTCACCGTACTAAGGCGGTGTCAAGAAGCAGACCGGGAA GAATTGAATTACTGGATCCGGCGGTACAGTGACGCCGAG |
| 25 | Quad 13 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG TTCCCACCCGAGGTCGCTGTGTTTGAGCCATCAGAAGCAGAGATCTCCCACACCCAAAAG GCCACACTGGTGTGCCTGGCCACAGGCTTCTACCCCGACCATGTGGAGCTGAGCTGGTGG GTGAATGGGAAGGAGGTGCACAGTGGGGTCAGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT GACGAGTGGACCCAGGATAGGGCCAAACCCGTCACCCAGATCGTCAGCGCCGAGGCCTGG GGTAGAGCAGACGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGGAACCCAAGTCCTGCGACAAGACCCACACCTGTCCCCCTTGTCCTGGAGGAGGT GGGAGCCTAACAGACAGAGAATGGGCAGAAGAGTGGAAACATCTTGACCATCTGTTAAAC TGCATAATGGACATGGTAGAAAAAACAAGGCGATCTCTCACCGTACTAAGGCGGTGTCAA GAAGCAGACCGGGAAGAATTGAATTACTGGATCCGGCGGTACAGTGACGCCGAG |
| 26 | Quad 14 | ATGGAGCTGGGGCTGAGCTGGGTGGTCCTGGCTGCTCTACTACAAGGTGTCCAG GCTCAGGTTCAGCTGGTTGAAAGCGGTGGTGCACTGGTTCAGCCTGGTGGTAGCCTGCGT CTGAGCTGTGCAGCAAGCGGTTTTCCGGTTAATCGTTATAGCATGCGTTGGTATCGTCAG GCACCGGGTAAAGAACGTGAATGGGTTGCAGGTATGAGCAGTGCCGGTGATCGTAGCAGC TATGAAGATAGCGTTAAAGGTCGTTTTACCATCAGCCGTGATGATGCACGTAATACCGTT TATCTGCAAATGAATAGCCTGAAACCGGAAGATACCGCAGTGTATTATTGCAATGTTAAC GTGGGCTTTGAATATTGGGGTCAGGGCACCCAGGTTACCGTTAGCAGCAAACTAACAGAC AGAGAATGGGCAGAAGAGTGGAAACATCTTGACCATCTGTTAAACTGCATAATGGACATG GTAGAAAAAACAAGGCGATCTCTCACCGTACTAAGGCGGTGTCAAGAAGCAGACCGGGAA GAATTGAATTACTGGATCCGGCGGTACAGTGACGCCGAG |
| 27 | Quad 15 | ATGGAGCTGGGGCTGAGCTGGGTGGTCCTGGCTGCTCTACTACAAGGTGTCCAG GCTCAGGTTCAGCTGGTTGAAAGCGGTGGTGCACTGGTTCAGCCTGGTGGTAGCCTGCGT CTGAGCTGTGCAGCAAGCGGTTTTCCGGTTAATCGTTATAGCATGCGTTGGTATCGTCAG GCACCGGGTAAAGAACGTGAATGGGTTGCAGGTATGAGCAGTGCCGGTGATCGTAGCAGC TATGAAGATAGCGTTAAAGGTCGTTTTACCATCAGCCGTGATGATGCACGTAATACCGTT TATCTGCAAATGAATAGCCTGAAACCGGAAGATACCGCAGTGTATTATTGCAATGTTAAC |
| | | GTGGGCTTTGAATATTGGGGTCAGGGCACCCAGGTTACCGTTAGCAGCAAAGGAGGAGGT GGGAGCCTAACAGACAGAGAATGGGCAGAAGAGTGGAAACATCTTGACCATCTGTTAAAC TGCATAATGGACATGGTAGAAAAAACAAGGCGATCTCTCACCGTACTAAGGCGGTGTCAA GAAGCAGACCGGGAAGAATTGAATTACTGGATCCGGCGGTACAGTGACGCCGAG |
| 28 | Quad 18 | ATGGAGCTGGGGCTGAGCTGGGTGGTCCTGGCTGCTCTACTACAAGGTGTCCAG GCTCAGGTTCAGCTGGTTGAAAGCGGTGGTGCACTGGTTCAGCCTGGTGGTAGCCTGCGT CTGAGCTGTGCAGCAAGCGGTTTTCCGGTTAATCGTTATAGCATGCGTTGGTATCGTCAG GCACCGGGTAAAGAACGTGAATGGGTTGCAGGTATGAGCAGTGCCGGTGATCGTAGCAGC TATGAAGATAGCGTTAAAGGTCGTTTTACCATCAGCCGTGATGATGCACGTAATACCGTT TATCTGCAAATGAATAGCCTGAAACCGGAAGATACCGCAGTGTATTATTGCAATGTTAAC GTGGGCTTTGAATATTGGGGTCAGGGCACCCAGGTTACCGTTAGCAGCAAACTAACAGAC AGAGAATGGGCAGAAGAGTGGAAACATCTTGACCATCTGTTAAACTGCATAATGGACATG GTAGAAAAAACAAGGCGATCTCTCACCGTACTAAGGCGGTGTCAAGAAGCAGACCGGGAA GAATTGAATTACTGGATCCGGCGGTACAGTGACGCCGAGGCACCTACTTCAAGTTCTACA AAGAAAACACAGCTACAACTGGAGCATTTACTGCTGGATTTACAGATGATTTTGAATGGA ATTAATAATTACAAGAATCCCAAACTCACCAGGATGCTCACATTTAAGTTTTACATGCCC |
| | | AAGAAGGCCACAGAACTGAAACATCTTCAGTGTCTAGAAGAAGAACTCAAACCTCTGGAG GAAGTGCTAAATTTAGCTCAAAGCAAAAACTTTCACTTAAGACCCAGGGACTTAATCAGC AATATCAACGTAATAGTTCTGGAACTAAAGGGATCTGAAACAACATTCATGTGTGAATAT GCTGATGAGACAGCAACCATTGTAGAATTTCTGAACAGATGGATTACCTTTTGTCAAAGC ATCATCTCAACACTGACT |
| 29 | Quad 19 | ATGGAGCTGGGGCTGAGCTGGGTGGTCCTGGCTGCTCTACTACAAGGTGTCCAG GCTCAGGTTCAGCTGGTTGAAAGCGGTGGTGCACTGGTTCAGCCTGGTGGTAGCCTGCGT CTGAGCTGTGCAGCAAGCGGTTTTCCGGTTAATCGTTATAGCATGCGTTGGTATCGTCAG GCACCGGGTAAAGAACGTGAATGGGTTGCAGGTATGAGCAGTGCCGGTGATCGTAGCAGC TATGAAGATAGCGTTAAAGGTCGTTTTACCATCAGCCGTGATGATGCACGTAATACCGTT TATCTGCAAATGAATAGCCTGAAACCGGAAGATACCGCAGTGTATTATTGCAATGTTAAC GTGGGCTTTGAATATTGGGGTCAGGGCACCCAGGTTACCGTTAGCAGCAAAGGAGGAGGT GGGAGCCTAACAGACAGAGAATGGGCAGAAGAGTGGAAACATCTTGACCATCTGTTAAAC TGCATAATGGACATGGTAGAAAAAACAAGGCGATCTCTCACCGTACTAAGGCGGTGTCAA GAAGCAGACCGGGAAGAATTGAATTACTGGATCCGGCGGTACAGTGACGCCGAGGCACCT |
| | | ACTTCAAGTTCTACAAAGAAAACACAGCTACAACTGGAGCATTTACTGCTGGATTTACAG ATGATTTTGAATGGAATTAATAATTACAAGAATCCCAAACTCACCAGGATGCTCACATTT AAGTTTTACATGCCCAAGAAGGCCACAGAACTGAAACATCTTCAGTGTCTAGAAGAAGAA CTCAAACCTCTGGAGGAAGTGCTAAATTTAGCTCAAAGCAAAAACTTTCACTTAAGACCC AGGGACTTAATCAGCAATATCAACGTAATAGTTCTGGAACTAAAGGGATCTGAAACAACA TTCATGTGTGAATATGCTGATGAGACAGCAACCATTGTAGAATTTCTGAACAGATGGATT ACCTTTTGTCAAAGCATCATCTCAACACTGACT |
| 30 | Quad 20 | ATGGAGACCCTCTTGGGCCTGCTTATCCTTTGGCTGCAGCTGCAATGGGTGAGC AGCAAACAGGAGGTGACACAGATTCCTGCAGCTCTGAGTGTCCCAGAAGGAGAAAACTTG GTTCTCAACTGCAGTTTCACTGATAGCGCTATTTACAACCTCCAGTGGTTTAGGCAGGAC CCTGGGAAAGGTCTCACATCTCTGTTGCTTATTACACCTTGGCAGAGAGAGCAAACAAGT GGAAGACTTAATGCCTCGCTGGATAAATCATCAGGACGTAGTACTTTATACATTGCAGCT TCTCAGCCTGGTGACTCAGCCACCTACCTCTGTGCTGTGAGGCCCCTGCTTGACGGAACA TACATACCTACATTTGGAAGAGGAACCAGCCTTATTGTTCATCCGTATATCCAGAACCCT GACCCTGCCGTGTACCAGCTGAGAGACTCTAAATCCAGTGACAAGTCTGTCTGCCTATTC ACCGATTTTGATTCTCAAACAAATGTGTCACAAAGTAAGGATTCTGATGTGTATATCACA |
| | | GACAAAACTGTGCTAGACATGAGGTCTATGGACTTCAAGAGCAACAGTGCTGTGGCCTGG AGCAACAAATCTGACTTTGCATGTGCAAACGCCTTCAACAACAGCATTATTCCAGAAGAC ACCTTCTTCCCCAGCCCAGAAAGTTCCGCCAGCACCAAGGGCCCCTCTGTGTTCCCTCTG GCCCCTTCCAGCAAGTCCACCTCTGGCGGAACAGCCGCTCTGGGCTGCCTCGTGAAGGAC TACTTCCCCGAGCCTGTGACCGTGTCCTGGAACTCTGGCGCTCTGACCAGCGGAGTGCAC ACCTTCCCTGCTGTGCTGCAGTCCTCCGGCCTGTACTCCCTGTCCTCCGTCGTGACCGTG CCTTCCAGCTCTCTGGGCACCCAGACCTACATCTGCAACGTGAACCACAAGCCCTCCAAC ACCAAGGTGGACAAGAAGGTGTTGCATGGCACACGTCAAGAAGAAATGATTGATCACAGA CTAACAGACAGAGAATGGGCAGAAGAGTGGAAACATCTTGACCATCTGTTAAACTGCATA ATGGACATGGTAGAAAAAACAAGGCGATCTCTCACCGTACTAAGGCGGTGTCAAGAAGCA GACCGGGAAGAATTGAATTACTGGATCCGGCGGTACAGTGACGCCGAG |
| 31 | Quad 21 | ATGGAGACCCTCTTGGGCCTGCTTATCCTTTGGCTGCAGCTGCAATGGGTGAGC AGCAAACAGGAGGTGACACAGATTCCTGCAGCTCTGAGTGTCCCAGAAGGAGAAAACTTG GTTCTCAACTGCAGTTTCACTGATAGCGCTATTTACAACCTCCAGTGGTTTAGGCAGGAC CCTGGGAAAGGTCTCACATCTCTGTTGCTTATTACACCTTGGCAGAGAGAGCAAACAAGT GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA TACATACCTACATTTGGAAGAGGAACCAGCCTTATTGTTCATCCGTATATCCAGAACCCT GACCCTGCCGTGTACCAGCTGAGAGACTCTAAATCCAGTGACAAGTCTGTCTGCCTATTC ACCGATTTTGATTCTCAAACAAATGTGTCACAAAGTAAGGATTCTGATGTGTATATCACA GACAAAACTGTGCTAGACATGAGGTCTATGGACTTCAAGAGCAACAGTGCTGTGGCCTGG AGCAACAAATCTGACTTTGCATGTGCAAACGCCTTCAACAACAGCATTATTCCAGAAGAC ACCTTCTTCCCCAGCCCAGAAAGTTCCGCCAGCACCAAGGGCCCCTCTGTGTTCCCTCTG GCCCCTTCCAGCAAGTCCACCTCTGGCGGAACAGCCGCTCTGGGCTGCCTCGTGAAGGAC TACTTCCCCGAGCCTGTGACCGTGTCCTGGAACTCTGGCGCTCTGACCAGCGGAGTGCAC ACCTTCCCTGCTGTGCTGCAGTCCTCCGGCCTGTACTCCCTGTCCTCCGTCGTGACCGTG CCTTCCAGCTCTCTGGGCACCCAGACCTACATCTGCAACGTGAACCACAAGCCCTCCAAC ACCAAGGTGGACAAGAAGGTGGGAGGAGGTGGGAGCTTGCATGGCACACGTCAAGAAGAA ATGATTGATCACAGACTAACAGACAGAGAATGGGCAGAAGAGTGGAAACATCTTGACCAT CTGTTAAACTGCATAATGGACATGGTAGAAAAAACAAGGCGATCTCTCACCGTACTAAGG CGGTGTCAAGAAGCAGACCGGGAAGAATTGAATTACTGGATCCGGCGGTACAGTGACGCC GAGGACTTAAAA |
| 32 | Quad 22 | ATGAGCCTCGGGCTCCTGTGCTGTGGGGCCTTTTCTCTCCTGTGGGCAGGTCCA GTGAATGCCGGTGTCACTCAGACCCCAAAATTCCAGGTCCTGAAGACAGGACAGAGCATG ACACTGCAGTGTGCCCAGGATATGAACCATGAATACATGTCCTGGTATCGACAAGACCCA GGCATGGGGCTGAGGCTGATTCATTACTCAGTTGCCATCCAGACAACTGACCAAGGAGAA GTCCCCAATGGCTACAATGTCTCCAGATCAACCATCGAGGATTTCCCGCTCAGGCTGCTG TCGGCTGCTCCCTCCCAGACATCTGTGTACTTCTGTGCCAGCAGTTACCTGGGGAACACC GGGGAGCTGTTTTTTGGAGAAGGCTCTAGGCTGACCGTACTGGAGGACCTGAAAAACGTG TTCCCACCCGAGGTCGCTGTGTTTGAGCCATCAGAAGCAGAGATCTCCCACACCCAAAAG GCCACACTGGTGTGCCTGGCCACAGGCTTCTACCCCGACCATGTGGAGCTGAGCTGGTGG GTGAATGGGAAGGAGGTGCACAGTGGGGTCAGCACAGACCCGCAGCCCCTCAAGGAGCAG CCCGCCCTCAATGACTCCAGATACGCTCTGAGCAGCCGCCTGAGGGTCTCGGCCACCTTC TGGCAGGACCCCCGCAACCACTTCCGCTGTCAAGTCCAGTTCTACGGGCTCTCGGAGAAT GACGAGTGGACCCAGGATAGGGCCAAACCCGTCACCCAGATCGTCAGCGCCGAGGCCTGG GGTAGAGCAGACACGGTGGCCGCTCCCTCCGTGTTCATCTTCCCACCTTCCGACGAGCAG CTGAAGTCCGGCACCGCTTCTGTCGTGTGCCTGCTGAACAACTTCTACCCCCGCGAGGCC |
| | | AAGGTGCAGTGGAAGGTGGACAACGCCCTGCAGTCCGGCAACTCCCAGGAATCCGTGACC GAGCAGGACTCCAAGGACAGCACCTACTCCCTGTCCTCCACCCTGACCCTGTCCAAGGCC GACTACGAGAAGCACAAGGTGTACGCCTGCGAAGTGACCCACCAGGGCCTGTCTAGCCCC GTGACCAAGTCTTTCAACCGGGGCGAGTGT |
| 33 | Quad 23 | ATGAACTTCGGGCTCAGCTTGATTTTCCTTGCCCTTATTTTAAAAGGTGTCCAG TGTGAGGTGCAACTGGTGGAGTCTGGGGGAGGCTTAGTGAAGCCTGGGGGGTCCCTGAAA CTCTCCTGTGCAGCCTCTGGACTCACTTTCAGTAGCTATGCCATGTCTTGGGTTCGCCAG ACTCCAGAGAAGAGGCTGGAGTGGGTCGCATCCATTAGTAGTGGTGGTTTCACCTACTAT CCAGACAGTGTGAAGGGCCGATTCACCATCTCCAGAGATAATGCCAGGAACATCCTGTAT CTGCAAATGAGCAGTCTGAGGTCTGAGGACACGGCCATGTATTACTGTGCAAGAGACGAG GTACGGGGGTACCTCGATGTCTGGGGCGCAGGGACCACGGTCACCGTTTCCTCGGCCAGC ACCAAGGGCCCCTCTGTGTTCCCTCTGGCCCCTTCCAGCAAGTCCACCTCTGGCGGAACA GCCGCTCTGGGCTGCCTCGTGAAGGACTACTTCCCCGAGCCTGTGACCGTGTCCTGGAAC TCTGGCGCTCTGACCAGCGGAGTGCACACCTTCCCTGCTGTGCTGCAGTCCTCCGGCCTG TACTCCCTGTCCTCCGTCGTGACCGTGCCTTCCAGCTCTCTGGGCACCCAGACCTACATC |
| | | TGCAACGTGAACCACAAGCCCTCCAACACCAAGGTGGACAAGAAGGTGCTAACAGACAGA GAATGGGCAGAAGAGTGGAAACATCTTGACCATCTGTTAAACTGCATAATGGACATGGTA GAAAAAACAAGGCGATCTCTCACCGTACTAAGGCGGTGTCAAGAAGCAGACCGGGAAGAA TTGAATTACTGGATCCGGCGGTACAGTGACGCCGAG |
| 34 | Quad 24 | ATGAACTTCGGGCTCAGCTTGATTTTCCTTGCCCTTATTTTAAAAGGTGTCCAG TGTGAGGTGCAACTGGTGGAGTCTGGGGGAGGCTTAGTGAAGCCTGGGGGGTCCCTGAAA CTCTCCTGTGCAGCCTCTGGACTCACTTTCAGTAGCTATGCCATGTCTTGGGTTCGCCAG ACTCCAGAGAAGAGGCTGGAGTGGGTCGCATCCATTAGTAGTGGTGGTTTCACCTACTAT CCAGACAGTGTGAAGGGCCGATTCACCATCTCCAGAGATAATGCCAGGAACATCCTGTAT CTGCAAATGAGCAGTCTGAGGTCTGAGGACACGGCCATGTATTACTGTGCAAGAGACGAG GTACGGGGGTACCTCGATGTCTGGGGCGCAGGGACCACGGTCACCGTTTCCTCGGCCAGC ACCAAGGGCCCCTCTGTGTTCCCTCTGGCCCCTTCCAGCAAGTCCACCTCTGGCGGAACA GCCGCTCTGGGCTGCCTCGTGAAGGACTACTTCCCCGAGCCTGTGACCGTGTCCTGGAAC TCTGGCGCTCTGACCAGCGGAGTGCACACCTTCCCTGCTGTGCTGCAGTCCTCCGGCCTG TACTCCCTGTCCTCCGTCGTGACCGTGCCTTCCAGCTCTCTGGGCACCCAGACCTACATC TGCAACGTGAACCACAAGCCCTCCAACACCAAGGTGGACAAGAAGGTGGGAGGAGGTGGG |
| | | AGCCTAACAGACAGAGAATGGGCAGAAGAGTGGAAACATCTTGACCATCTGTTAAACTGC ATAATGGACATGGTAGAAAAAACAAGGCGATCTCTCACCGTACTAAGGCGGTGTCAAGAA GCAGACCGGGAAGAATTGAATTACTGGATCCGGCGGTACAGTGACGCCGAG |
| 35 | Quad 25 | ATGAAATACCTATTGCCTACGGCAGCCGCTGGATTGTTATTACTCGCGGCCCAG CCGGCCATGGCGGCCTACAAAGATATCCAGATGACACAGACTACATCCTCCCTGTCTGCC TCTCTGGGAGACAGAGTCACCATCAGTTGCAGTGCAAGTCAGGGCATTAGCAATTATTTA AACTGGTATCAGCAGAAACCAGATGGAACTGTTAAACTCCTGATCTATTACACATCAAGT TTACACTCAGGAGTCCCATCAAGGTTCAGTGGCAGTGGGTCTGGGACAGATTATTCTCTC ACCATCAGCAACCTGGAACCTGAAGATATTGCCACTTATTATTGTCAGCAGTATAGCAAG CTTCCGTACACGTTCGGAGGGGGGACCAAGCTGGAAATAAAACGTACGGTGGCCGCTCCC TCCGTGTTCATCTTCCCACCTTCCGACGAGCAGCTGAAGTCCGGCACCGCTTCTGTCGTG TGCCTGCTGAACAACTTCTACCCCCGCGAGGCCAAGGTGCAGTGGAAGGTGGACAACGCC CTGCAGTCCGGCAACTCCCAGGAATCCGTGACCGAGCAGGACTCCAAGGACAGCACCTAC TCCCTGTCCTCCACCCTGACCCTGTCCAAGGCCGACTACGAGAAGCACAAGGTGTACGCC TGCGAAGTGACCCACCAGGGCCTGTCTAGCCCCGTGACCAAGTCTTTCAACCGGGGCGAG TGT |
| 36 | Quad 26 | ATGAACTTCGGGCTCAGCTTGATTTTCCTTGCCCTTATTTTAAAAGGTGTCCAG TGTGAGGTGCAACTGGTGGAGTCTGGGGGAGGCTTAGTGAAGCCTGGGGGGTCCCTGAAA CTCTCCTGTGCAGCCTCTGGACTCACTTTCAGTAGCTATGCCATGTCTTGGGTTCGCCAG ACTCCAGAGAAGAGGCTGGAGTGGGTCGCATCCATTAGTAGTGGTGGTTTCACCTACTAT CCAGACAGTGTGAAGGGCCGATTCACCATCTCCAGAGATAATGCCAGGAACATCCTGTAT CTGCAAATGAGCAGTCTGAGGTCTGAGGACACGGCCATGTATTACTGTGCAAGAGACGAG GTACGGGGGTACCTCGATGTCTGGGGCGCAGGGACCACGGTCACCGTTTCCTCGGCCAGC ACCAAGGGCCCCTCTGTGTTCCCTCTGGCCCCTTCCAGCAAGTCCACCTCTGGCGGAACA GCCGCTCTGGGCTGCCTCGTGAAGGACTACTTCCCCGAGCCTGTGACCGTGTCCTGGAAC TCTGGCGCTCTGACCAGCGGAGTGCACACCTTCCCTGCTGTGCTGCAGTCCTCCGGCCTG TACTCCCTGTCCTCCGTCGTGACCGTGCCTTCCAGCTCTCTGGGCACCCAGACCTACATC TGCAACGTGAACCACAAGCCCTCCAACACCAAGGTGGACAAGAAGGTGGAACCCAAGTCC TGCGACAAGACCCACACCTGTCCCCCTTGTCCTCTAACAGACAGAGAATGGGCAGAAGAG TGGAAACATCTTGACCATCTGTTAAACTGCATAATGGACATGGTAGAAAAAACAAGGCGA TCTCTCACCGTACTAAGGCGGTGTCAAGAAGCAGACCGGGAAGAATTGAATTACTGGATC |
| | | CGGCGGTACAGTGACGCCGAG |
| 37 | Quad 27 | ATGAACTTCGGGCTCAGCTTGATTTTCCTTGCCCTTATTTTAAAAGGTGTCCAG TGTGAGGTGCAACTGGTGGAGTCTGGGGGAGGCTTAGTGAAGCCTGGGGGGTCCCTGAAA CTCTCCTGTGCAGCCTCTGGACTCACTTTCAGTAGCTATGCCATGTCTTGGGTTCGCCAG ACTCCAGAGAAGAGGCTGGAGTGGGTCGCATCCATTAGTAGTGGTGGTTTCACCTACTAT CCAGACAGTGTGAAGGGCCGATTCACCATCTCCAGAGATAATGCCAGGAACATCCTGTAT CTGCAAATGAGCAGTCTGAGGTCTGAGGACACGGCCATGTATTACTGTGCAAGAGACGAG GTACGGGGGTACCTCGATGTCTGGGGCGCAGGGACCACGGTCACCGTTTCCTCGGCCAGC ACCAAGGGCCCCTCTGTGTTCCCTCTGGCCCCTTCCAGCAAGTCCACCTCTGGCGGAACA GCCGCTCTGGGCTGCCTCGTGAAGGACTACTTCCCCGAGCCTGTGACCGTGTCCTGGAAC TCTGGCGCTCTGACCAGCGGAGTGCACACCTTCCCTGCTGTGCTGCAGTCCTCCGGCCTG TACTCCCTGTCCTCCGTCGTGACCGTGCCTTCCAGCTCTCTGGGCACCCAGACCTACATC TGCAACGTGAACCACAAGCCCTCCAACACCAAGGTGGACAAGAAGGTGGAACCCAAGTCC TGCGACAAGACCCACACCTGTCCCCCTTGTCCTGGAGGAGGTGGGAGCCTAACAGACAGA GAATGGGCAGAAGAGTGGAAACATCTTGACCATCTGTTAAACTGCATAATGGACATGGTA GAAAAAACAAGGCGATCTCTCACCGTACTAAGGCGGTGTCAAGAAGCAGACCGGGAAGAA |
| | | TTGAATTACTGGATCCGGCGGTACAGTGACGCCGAG |
| 38 | Quad 28 | ATGGGATGGTCTTGTATAATTCTGTTCCTGGTGGCAACAGCAACAGGAGTGCAT AGCGAGGTGCAGCTGGTGGAGTCTGGGGGAGGCTTGGTACAGCCCGGCAGGTCCCTGAGA CTCTCCTGTGCGGCCTCTGGATTCACCTTTGATGATTATGCCATGCACTGGGTCCGGCAA GCTCCAGGGAAGGGCCTGGAATGGGTCTCAGCTATCACTTGGAATAGTGGTCACATAGAC TATGCGGACTCTGTGGAGGGCCGATTCACCATCTCCAGAGACAACGCCAAGAACTCCCTG TATCTGCAAATGAACAGTCTGAGAGCTGAGGATACGGCCGTATATTACTGTGCGAAAGTC TCGTACCTTAGCACCGCGTCCTCCCTTGACTATTGGGGCCAAGGTACCCTGGTCACCGTC TCGAGTGCCAGCACCAAGGGCCCCTCTGTGTTCCCTCTGGCCCCTTCCAGCAAGTCCACC TCTGGCGGAACAGCCGCTCTGGGCTGCCTCGTGAAGGACTACTTCCCCGAGCCTGTGACC GTGTCCTGGAACTCTGGCGCTCTGACCAGCGGAGTGCACACCTTCCCTGCTGTGCTGCAG TCCTCCGGCCTGTACTCCCTGTCCTCCGTCGTGACCGTGCCTTCCAGCTCTCTGGGCACC CAGACCTACATCTGCAACGTGAACCACAAGCCCTCCAACACCAAGGTGGACAAGAAGGTG TTGCATGGCACACGTCAAGAAGAAATGATTGATCACAGACTAACAGACAGAGAATGGGCA GAAGAGTGGAAACATCTTGACCATCTGTTAAACTGCATAATGGACATGGTAGAAAAAACA AGGCGATCTCTCACCGTACTAAGGCGGTGTCAAGAAGCAGACCGGGAAGAATTGAATTAC |
| | | TGGATCCGGCGGTACAGTGACGCCGAG |
| 39 | Quad 29 | ATGGGATGGTCTTGTATAATTCTGTTCCTGGTGGCAACAGCAACAGGAGTGCAT AGCGAGGTGCAGCTGGTGGAGTCTGGGGGAGGCTTGGTACAGCCCGGCAGGTCCCTGAGA CTCTCCTGTGCGGCCTCTGGATTCACCTTTGATGATTATGCCATGCACTGGGTCCGGCAA GCTCCAGGGAAGGGCCTGGAATGGGTCTCAGCTATCACTTGGAATAGTGGTCACATAGAC TATGCGGACTCTGTGGAGGGCCGATTCACCATCTCCAGAGACAACGCCAAGAACTCCCTG TATCTGCAAATGAACAGTCTGAGAGCTGAGGATACGGCCGTATATTACTGTGCGAAAGTC TCGTACCTTAGCACCGCGTCCTCCCTTGACTATTGGGGCCAAGGTACCCTGGTCACCGTC TCGAGTGCCAGCACCAAGGGCCCCTCTGTGTTCCCTCTGGCCCCTTCCAGCAAGTCCACC TCTGGCGGAACAGCCGCTCTGGGCTGCCTCGTGAAGGACTACTTCCCCGAGCCTGTGACC GTGTCCTGGAACTCTGGCGCTCTGACCAGCGGAGTGCACACCTTCCCTGCTGTGCTGCAG TCCTCCGGCCTGTACTCCCTGTCCTCCGTCGTGACCGTGCCTTCCAGCTCTCTGGGCACC CAGACCTACATCTGCAACGTGAACCACAAGCCCTCCAACACCAAGGTGGACAAGAAGGTG GGAGGAGGTGGGAGCTTGCATGGCACACGTCAAGAAGAAATGATTGATCACAGACTAACA GACAGAGAATGGGCAGAAGAGTGGAAACATCTTGACCATCTGTTAAACTGCATAATGGAC ATGGTAGAAAAAACAAGGCGATCTCTCACCGTACTAAGGCGGTGTCAAGAAGCAGACCGG |
| | | GAAGAATTGAATTACTGGATCCGGCGGTACAGTGACGCCGAG |
| 40 | Quad 30 | ATGGACATGAGGGTCCCTGCTCAGCTCCTGGGACTCCTGCTGCTCTGGCTCCCA GATACCAGATGTGACATCCAGATGACCCAGTCTCCATCCTCCCTGTCTGCATCTGTAGGG GACAGAGTCACCATCACTTGTCGGGCAAGTCAGGGCATCAGAAATTACTTAGCCTGGTAT CAGCAAAAACCAGGGAAAGCCCCTAAGCTCCTGATCTATGCTGCATCCACTTTGCAATCA GGGGTCCCATCTCGGTTCAGTGGCAGTGGATCTGGGACAGATTTCACTCTCACCATCAGC AGCCTACAGCCTGAAGATGTTGCAACTTATTACTGTCAAAGGTATAACCGTGCACCGTAT ACTTTTGGCCAGGGGACCAAGGTGGAAATCAAACGTACGGTGGCCGCTCCCTCCGTGTTC ATCTTCCCACCTTCCGACGAGCAGCTGAAGTCCGGCACCGCTTCTGTCGTGTGCCTGCTG AACAACTTCTACCCCCGCGAGGCCAAGGTGCAGTGGAAGGTGGACAACGCCCTGCAGTCC GGCAACTCCCAGGAATCCGTGACCGAGCAGGACTCCAAGGACAGCACCTACTCCCTGTCC TCCACCCTGACCCTGTCCAAGGCCGACTACGAGAAGCACAAGGTGTACGCCTGCGAAGTG ACCCACCAGGGCCTGTCTAGCCCCGTGACCAAGTCTTTCAACCGGGGCGAGTGT |
| 41 | Quad 31 | ATGGGATGGTCTTGTATAATTCTGTTCCTGGTGGCAACAGCAACAGGAGTGCAT AGCGAGGTCCAACTTGTCGAAAGTGGCGGCGGTTTGGTTCAACCTGGAGGTTCACTTCGA |
| | | CTGTCATGTGCAGCGAGCGGTTATACATTTACGAATTATGGCATGAATTGGGTTAGACAG GCACCAGGAAAGGGACTGGAGTGGGTAGGCTGGATCAATACCTACACAGGAGAACCAACG TATGCCGCAGACTTCAAACGACGGTTTACATTTTCCTTGGATACCTCTAAGTCTACAGCG TATCTCCAAATGAATTCACTTCGAGCGGAAGATACCGCGGTCTACTATTGCGCCAAATAC CCTCATTATTATGGGTCATCTCACTGGTATTTCGATGTCTGGGGTCAGGGAACACTGGTA ACCGTGTCATCCGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGTTGCATGGCACACGTCAAGAAGAAATGATTGATCACAGACTAACAGACAGAGAA TGGGCAGAAGAGTGGAAACATCTTGACCATCTGTTAAACTGCATAATGGACATGGTAGAA AAAACAAGGCGATCTCTCACCGTACTAAGGCGGTGTCAAGAAGCAGACCGGGAAGAATTG AATTACTGGATCCGGCGGTACAGTGACGCCGAG |
| 42 | Quad 32 | ATGGGATGGTCTTGTATAATTCTGTTCCTGGTGGCAACAGCAACAGGAGTGCAT AGCGAGGTCCAACTTGTCGAAAGTGGCGGCGGTTTGGTTCAACCTGGAGGTTCACTTCGA |
| | | CTGTCATGTGCAGCGAGCGGTTATACATTTACGAATTATGGCATGAATTGGGTTAGACAG GCACCAGGAAAGGGACTGGAGTGGGTAGGCTGGATCAATACCTACACAGGAGAACCAACG TATGCCGCAGACTTCAAACGACGGTTTACATTTTCCTTGGATACCTCTAAGTCTACAGCG TATCTCCAAATGAATTCACTTCGAGCGGAAGATACCGCGGTCTACTATTGCGCCAAATAC CCTCATTATTATGGGTCATCTCACTGGTATTTCGATGTCTGGGGTCAGGGAACACTGGTA ACCGTGTCATCCGCCAGCACCAAGGGCCCCTCTGTGTTCCCTCTGGCCCCTTCCAGCAAG TCCACCTCTGGCGGAACAGCCGCTCTGGGCTGCCTCGTGAAGGACTACTTCCCCGAGCCT GTGACCGTGTCCTGGAACTCTGGCGCTCTGACCAGCGGAGTGCACACCTTCCCTGCTGTG CTGCAGTCCTCCGGCCTGTACTCCCTGTCCTCCGTCGTGACCGTGCCTTCCAGCTCTCTG GGCACCCAGACCTACATCTGCAACGTGAACCACAAGCCCTCCAACACCAAGGTGGACAAG AAGGTGGGAGGAGGTGGGAGCTTGCATGGCACACGTCAAGAAGAAATGATTGATCACAGA CTAACAGACAGAGAATGGGCAGAAGAGTGGAAACATCTTGACCATCTGTTAAACTGCATA ATGGACATGGTAGAAAAAACAAGGCGATCTCTCACCGTACTAAGGCGGTGTCAAGAAGCA GACCGGGAAGAATTGAATTACTGGATCCGGCGGTACAGTGACGCCGAG |
| 43 | Quad 33 | ATGGACATGAGGGTCCCTGCTCAGCTCCTGGGGCTCCTGCAGCTCTGGCTCTCA GGTGCCAGATGTGACATCCAAATGACCCAGAGTCCTTCCAGCCTCAGTGCGTCAGTGGGA |
| | | GATCGAGTGACGATAACGTGTTCTGCCAGCCAAGACATTTCCAACTATCTTAATTGGTAC CAGCAGAAACCGGGAAAGGCCCCGAAAGTGCTCATATACTTTACCAGCAGTCTTCACTCT GGAGTTCCTAGCCGGTTTAGCGGCTCAGGTAGTGGCACCGATTTCACTCTGACCATTAGT TCTCTTCAACCGGAAGATTTTGCAACCTACTATTGTCAGCAGTATTCAACGGTACCTTGG ACCTTCGGCCAAGGCACCAAAGTCGAGATTAAGCGTACGGTGGCCGCTCCCTCCGTGTTC ATCTTCCCACCTTCCGACGAGCAGCTGAAGTCCGGCACCGCTTCTGTCGTGTGCCTGCTG AACAACTTCTACCCCCGCGAGGCCAAGGTGCAGTGGAAGGTGGACAACGCCCTGCAGTCC GGCAACTCCCAGGAATCCGTGACCGAGCAGGACTCCAAGGACAGCACCTACTCCCTGTCC TCCACCCTGACCCTGTCCAAGGCCGACTACGAGAAGCACAAGGTGTACGCCTGCGAAGTG ACCCACCAGGGCCTGTCTAGCCCCGTGACCAAGTCTTTCAACCGGGGCGAGTGT |
| 44 | Quad 34 | ATGGATATGCGCGTCCCGGCACAGCTGCTCGGCTTGTTGTTGCTGTGGTTGAGA GCTAGGTGCGATATACAGATGACTCAGTCCCCTTCCAGTCTTTCAGCCAGTGTCGGCGAC CGGGTTACCATTACTTGTCGGGCAAGTCAATCTATAGATAGTTATTTGCATTGGTATCAA CAAAAACCAGGCAAAGCGCCTAAGTTGTTGATATATTCCGCATCTGAACTGCAATCAGGC GTTCCTTCACGCTTTTCTGGAAGCGGCAGCGGAACCGATTTCACTCTTACCATAAGTAGT |
| | | CTCCAGCCGGAGGATTTTGCTACATACTATTGTCAACAAGTAGTGTGGCGACCGTTCACC TTCGGACAGGGGACAAAAGTAGAAATCAAGCGGGGAGGAGGTGGGAGCTTGCATGGCACA CGTCAAGAAGAAATGATTGATCACAGACTAACAGACAGAGAATGGGCAGAAGAGTGGAAA CATCTTGACCATCTGTTAAACTGCATAATGGACATGGTAGAAAAAACAAGGCGATCTCTC ACCGTACTAAGGCGGTGTCAAGAAGCAGACCGGGAAGAATTGAATTACTGGATCCGGCGG TACAGTGACGCCGAG |
| 45 | Quad 36 | ATGGAGTTTGGCCTCAGTTGGTTGTTTTTGGTAGCGAAAATTAAAGTACAGTGT GAAGTCCAACTCCTGGAGAGCGGGGGGGGTCTGGTACAACCAGGCGGCTCACTGCGGCTT AGCTGCGCAGCCTCCGGGTTCACGTTCGCACATGAAACGATGGTGTGGGTGCGCCAGGCA CCGGGGAAGGGACTCGAATGGGTCTCACATATACCTCCTGACGGTCAGGATCCTTTTTAC GCGGACTCTGTGAAGGGACGATTCACAATAAGTAGAGACAATAGTAAGAACACCCTTTAT TTGCAGATGAACAGTCTGCGGGCGGAAGATACAGCAGTATATCATTGTGCCCTGCTGCCC AAACGAGGTCCGTGGTTTGACTATTGGGGACAGGGGACTCTCGTTACTGTAAGCTCCGGA GGAGGTGGGAGCTTGCATGGCACACGTCAAGAAGAAATGATTGATCACAGACTAACAGAC AGAGAATGGGCAGAAGAGTGGAAACATCTTGACCATCTGTTAAACTGCATAATGGACATG GTAGAAAAAACAAGGCGATCTCTCACCGTACTAAGGCGGTGTCAAGAAGCAGACCGGGAA |
| | | GAATTGAATTACTGGATCCGGCGGTACAGTGACGCCGAG |
| 46 | Quad 38 | ATGGACATGAGGGTCCCCGCTCAGCTCCTGGGGCTCCTGCTACTCTGGCTCCGA GCCAGATGTGATATACAGATGACCCAATCACCAAGCAGCTTGTCCGCTTCAGTGGGCGAC AGGGTAACTATAACATGCCGCGCAAGCCAATGGATAGGTCCAGAACTCTCATGGTACCAA CAAAAACCAGGGAAAGCGCCGAAACTGCTTATCTATCACACAAGCATTTTGCAATCTGGG GTACCTAGTCGATTCAGTGGCTCTGGCAGTGGGACTGACTTTACACTCACCATAAGTTCT CTCCAACCAGAGGACTTTGCAACATACTATTGTCAGCAATATATGTTTCAACCACGCACC TTTGGACAAGGCACAAAAGTTGAAATCCGCGGAGGAGGTGGGAGCTTGCATGGCACACGT CAAGAAGAAATGATTGATCACAGACTAACAGACAGAGAATGGGCAGAAGAGTGGAAACAT CTTGACCATCTGTTAAACTGCATAATGGACATGGTAGAAAAAACAAGGCGATCTCTCACC GTACTAAGGCGGTGTCAAGAAGCAGACCGGGAAGAATTGAATTACTGGATCCGGCGGTAC AGTGACGCCGAG |
| 47 | Quad 40 | ATGGACATGAGGGTCCCCGCTCAGCTCCTGGGGCTCCTGCTACTCTGGCTCCGA GCCAGATGTGATATTCAAATGACACAGTCACCAACGAGTCTTTCCGCGAGCGTTGGGGAC CGAGTGACAATAACTTGTCGAGCCTCTCAGTGGATTGGCAACTTGCTGGACTGGTATCAG CAAAAGCCGGGAGAAGCCCCGAAGCTGCTCATATACTATGCTTCCTTCCTCCAGAGTGGA |
| | | GTACCTAGCAGATTCAGCGGGGGGGGATTCGGGACTGATTTCACTCTTACAATCAGCTCT CTTCAACCCGAGGACTTCGCAACGTACTACTGTCAACAAGCTAACCCTGCGCCGCTTACT TTCGGACAAGGCACTAAGGTCGAAATTAAGCGAGGAGGAGGTGGGAGCTTGCATGGCACA CGTCAAGAAGAAATGATTGATCACAGACTAACAGACAGAGAATGGGCAGAAGAGTGGAAA CATCTTGACCATCTGTTAAACTGCATAATGGACATGGTAGAAAAAACAAGGCGATCTCTC ACCGTACTAAGGCGGTGTCAAGAAGCAGACCGGGAAGAATTGAATTACTGGATCCGGCGG TACAGTGACGCCGAG |
| 48 | Quad 42 | ATGGACATGAGGGTCCCCGCTCAGCTCCTGGGGCTCCTGCTACTCTGGCTCCGA GCCAGATGTGATATACAGATGACCCAATCACCAAGCAGCTTGTCCGCTTCAGTGGGCGAC AGGGTAACTATAACATGCCGCGCAAGCCAATGGATAGGTCCAGAACTCTCATGGTACCAA CAAAAACCAGGGAAAGCGCCGAAACTGCTTATCTATCACACAAGCATTTTGCAATCTGGG GTACCTAGTCGATTCAGTGGCTCTGGCAGTGGGACTGACTTTACACTCACCATAAGTTCT CTCCAACCAGAGGACTTTGCAACATACTATTGTCAGCAATATATGTTTCAACCACGCACC TTTGGACAAGGCACAAAAGTTGAAATCCGCGGAGGAGGTGGGAGCTTGCATGGCACACGT CAAGAAGAAATGATTGATCACAGACTAACAGACAGAGAATGGGCAGAAGAGTGGAAACAT CTTGACCATCTGTTAAACTGCATAATGGACATGGTAGAAAAAACAAGGCGATCTCTCACC |
| | | GTACTAAGGCGGTGTCAAGAAGCAGACCGGGAAGAATTGAATTACTGGATCCGGCGGTAC AGTGACGCCGAGGACTTAAAAGGTGGAGGAGGTAGCGATATTCAAATGACACAGTCACCA ACGAGTCTTTCCGCGAGCGTTGGGGACCGAGTGACAATAACTTGTCGAGCCTCTCAGTGG ATTGGCAACTTGCTGGACTGGTATCAGCAAAAGCCGGGAGAAGCCCCGAAGCTGCTCATA TACTATGCTTCCTTCCTCCAGAGTGGAGTACCTAGCAGATTCAGCGGGGGGGGATTCGGG ACTGATTTCACTCTTACAATCAGCTCTCTTCAACCCGAGGACTTCGCAACGTACTACTGT CAACAAGCTAACCCTGCGCCGCTTACTTTCGGACAAGGCACTAAGGTCGAAATTAAGCGA |
| 49 | Quad 44 | ATGGACATGAGGGTCCCCGCTCAGCTCCTGGGGCTCCTGCTACTCTGGCTCCGA GCCAGATGTGACATTCAGATGACTCAGTCACCATCCTCATTGTCTGCATCAGTTGGTGAC CGAGTTACGATCACATGTCGAGCAAGCCAAAATATAGATTCCAGACTTTCATGGTACCAG CAGAAGCCTGGTAAAGCGCCGAAACTCCTCATATATCGCACGAGCGTATTGCAATCTGGT GTGCCTTCTCGATTTTCAGGATCTGGGTCTGGCACTGACTTCACCTTGACAATATCTTCT CTTCAGCCCGAAGATTTCGCTACCTACTACTGCCAACAATGGGACATGTTTCCTCTGACC TTCGGACAGGGTACAAAGGTCGAGATTAAACGGGGAGGAGGTGGGAGCTTGCATGGCACA CGTCAAGAAGAAATGATTGATCACAGACTAACAGACAGAGAATGGGCAGAAGAGTGGAAA |
| | | CATCTTGACCATCTGTTAAACTGCATAATGGACATGGTAGAAAAAACAAGGCGATCTCTC ACCGTACTAAGGCGGTGTCAAGAAGCAGACCGGGAAGAATTGAATTACTGGATCCGGCGG TACAGTGACGCCGAG |
| 50 | Quad 46 | ATGGACATGAGGGTCCCCGCTCAGCTCCTGGGGCTCCTGCTACTCTGGCTCCGA GCCAGATGTGACATTCAGATGACTCAGTCACCATCCTCATTGTCTGCATCAGTTGGTGAC CGAGTTACGATCACATGTCGAGCAAGCCAAAATATAGATTCCAGACTTTCATGGTACCAG CAGAAGCCTGGTAAAGCGCCGAAACTCCTCATATATCGCACGAGCGTATTGCAATCTGGT GTGCCTTCTCGATTTTCAGGATCTGGGTCTGGCACTGACTTCACCTTGACAATATCTTCT CTTCAGCCCGAAGATTTCGCTACCTACTACTGCCAACAATGGGACATGTTTCCTCTGACC TTCGGACAGGGTACAAAGGTCGAGATTAAACGGGGAGGAGGTGGGAGCTTGCATGGCACA CGTCAAGAAGAAATGATTGATCACAGACTAACAGACAGAGAATGGGCAGAAGAGTGGAAA CATCTTGACCATCTGTTAAACTGCATAATGGACATGGTAGAAAAAACAAGGCGATCTCTC ACCGTACTAAGGCGGTGTCAAGAAGCAGACCGGGAAGAATTGAATTACTGGATCCGGCGG TACAGTGACGCCGAGGACTTAAAAGGTGGAGGAGGTAGCGATATACAGATGACTCAATCC CCTTCATCCCTCTCAGCTTCCGTAGGGGACAGAGTTACTATAACGTGTCGAGCTAGTCAA GACATAGGTGATCGCCTGAGGTGGTATCAGCAAAAACCGGGTAAAGCACCTAAACTCCTC |
| | | ATATATCATGGTTCCAGGTTGGAGTCAGGCGTGCCGTCACGATTCTCTGGGTCACGCTCT GGCACTGACTTCACATTGACGATTAGTTCTCTCCAGCCCGAAGACTTCGCCACCTACTAC TGTCAACAGCAATGGTTTCGCCCGTATACTTTTGGGCAGGGTACAAAGGTTGAGATTAAA CGG |

TABLE 4

Amino acid sequences of binding moieties, domains and peptides

| SEQ ID NO: | Domain/petptide | Product* | Domain/peptide Amino Acid Sequence |
|---|---|---|---|
| 51 | Anti-TNF alpha H | Humira™ | EVQLVESGGGLVQPGRSLRLSCAASGFTFDDYAMHWVRQAPGKGLEWVSAITWNSGHIDY ADSVEGRFTISRDNAKNSLYLQMNSLRAEDTAVYYCAKVSYLSTASSLDYWGQGTLVTVS S |
| 52 | Anti TNF alpha VL | Humira™ | DIQMTQSPSSLSASVGDRVTITCRASQGIRNYLAWYQQKPGKAPKLLIYAASTLQSGVPS RFSGSGSGTDFTLTISSLQPEDVATYYCQRYNRAPYTFGQGTKVEIKR |
| 53 | Anti-CD20 VH | Rituximab | QVQLQQPGAELVKPGASVKMSCKASGYTFTSYNMHWVKQTPGRGLEWIGAIYPGNGDTSY NQKFKGKATLTADKSSSTAYMQLSSLTSEDSAVYYCARSTYYGGDWYFNVWGAGTTVTVS A |
| 54 | Anti-CD20 VL | Rituximab | QIVLSQSPAILSASPGEKVTMTCRASSSVSYIHWFQQKPGSSPKPWIYATSNLASGVPVR FSGSGSGTSYSLTISRVEAEDAATYYCQQWTSNPPTFGGGTKLEIKR |
| 55 | Anti-VEGF VH | Avastin™ | EVQLVESGGGLVQPGGSLRLSCAASGYTFTNYGMNWVRQAPGKGLEWVGWINTYTGEPTY AADFKRRFTFSLDTSKSTAYLQMNSLRAEDTAVYYCAKYPHYYGSSHWYFDVWGQGTLVT VSS |
| 56 | Anti-VEGF VL | Avastin™ | DIQMTQSPSSLSASVGDRVTITCSASQDISNYLNWYQQKPGKAPKVLIYFTSSLHSGVPS RFSGSGSGTDFTLTISSLQPEDFATYYCQQYSTVPWTFGQGTKVEIKR |
| 57 | Anti-HER2 VH | Herceptin™ | EVQLVESGGGLVQPGGSLRLSCAASGFTFTDYTMDWVRQAPGKGLEWVADVNPNSGGSIY NQRFKGRFTLSVDRSKNTLYLQMNSLRAEDTAVYYCARNLGPSFYFDYWGQGTLVTVSS |
| 58 | Anti-HER2 VL | Herceptin™ | DIQMTQSPSSLSASVGDRVTITCKASQDVSIGVAWYQQKPGKAPKLLIYSASYRYTGVPS RFSGSGSGTDFTLTISSLQPEDFATYYCQQYYIYPYTFGQGTKVEIKR |
| 59 | Anti-IL6R VH | Actemra™ | EVQLQESGPGLVRPSQTLSLTCTVSGYSITSDHAWSWVRQPPGRGLEWIGYISYSGITTY NPSLKSRVTMLRDTSKNQFSLRLSSVTAADTAVYYCARSLARTTAMDYWGQGSLVTVSS |
| 60 | Anti-IL6R VL | Actemra™ | DIQMTQSPSSLSASVGDRVTITCRASQDISSYLNWQQKPGKAPKLLIYYTSRLHSGVPSRFS GSGSGTDFTFTISSLQPEDIATYYCQQGNTLPYTFGQGTKVEIKR |
| 61 | Anti-PD-1 VH | Nivolumab | QVQLVESGGGVVQPGRSLRLDCKASGITFSNSGMHWVRQAPGKGLEWVAVIWYDGSKRYY ADSVKGRFTISRDNSKNTLFLQMNSLRAEDTAVYYCATNDDYWGQGTLVTVSS |
| 62 | Anti-PD-1 VL | Nivolumab | EIVLTQSPATLSLSPGERATLSCRASQSVSSYLAWYQQKPGQAPRLLIYDASNRATGIPARFS GSGSGTDFTLTISSLEPEDFAVYYCQQSSNWPRTFGQGTKVEIKR |
| 63 | Anti-CTLA4 VH | Ipilimumab | QVQLVESGGGVVQPGRSLRLSCAASGFTFSSYTMHWVRQAPGKGLEWVTFISYDGNNKYY ADSVKGRFTISRDNSKNTLYLQMNSLRAEDTAIYYCARTGWLGPFDYWGQGTLVTVSS |
| 64 | Anti-CTLA4 VL | Ipilimumab | EIVLTQSPGTLSLSPGERATLSCRASQSVGSSYLAWYQQKPGQAPRLLIYGAFSRATGIP |
| | | | DRFSGSGSGTDFTLTISRLEPEDFAVYYCQQYGSSPWTFGQGTKVEIKR |
| 65 | Anti-TNFR1 dAb | DOM1h-131-206 VH | EVQLLESGGGLVQPGGSLRLSCAASGFTFAHETMVWVRQAPGKGLEWVSHIPPDGQDPFY ADSVKGRFTISRDNSKNTLYLQMNSLRAEDTAVYHCALLPKRGPWFDYWGQGTLVTVSS |
| 66 | Anti-TNFα dAb | TAR 1-5- 19 Vk | DIQMTQSPSSLSASVGDRVTITCRASQSIDSYLHUVI′QQKPGKAPKLLIYSASELQSGVPSRFS GSGSGTDFTLTISSLQPEDFATYYCQQVVWRPFTFGQGTKVEIKR |
| 67 | Anti-VEGF dAb | TAR15-10 Vk | DIQMTQSPSSLSASVGDRVTITCRASQWIGPELSWYQQKPGKAPKLLIYHTSILQSGVPSRFS GSGSGTDFTLTISSLQPEDFATYYCQQYMFQPRTFGQGTKVEIR |
| 68 | Anti-EGFR dAb | DOM16-39-109 Vk | DIQMTQSPTSLSASVGDRVTITCRASQWIGNLLDWYQQKPGEAPKLLIYYASFLQSGVPSRFS GGGFGTDFTLTISSLQPEDFATYYCQQANPAPLTFGQGTKVEIKR |
| 69 | Anti-CD38 dAb | DOM 11-3 Vk | DIQMTQSPSSLSASVGDRVTITCRASQNIDSRLSWYQQKPGKAPKLLIYRTSVLQSGVPSRFS GSGSGTDFTLTISSLQPEDFATYYCQQWDMFPLTFGQGTKVEIKR |
| 70 | Anti-CD 138 dAb | DOM 12-45 Vk | DIQMTQSPSSLSASVGDRVTITCRASQDIGDRLRWYQQKPGKAPKLLIYHGSRLESGVPSRFS GSRSGTDFTLTISSLQPEDFATYYCQQQWFRPYTFGQGTKVEIKR |
| 71 | Anti-NY-ESO-1 Vα | | KQEVTQIPAALSVPEGENLVLNCSFTDSAIYNLQWFRQDPGKGLTSLLLITPWQREQTSGRLN ASLDKSSGRSTLYIAASQPGDSATYLCAVRPLLDGTYIPTFGRGTSLIVHPY |
| 72 | Anti-NY-ESO-1 Vβ | | NAGVTQTPKFQVLKTGQSMTLQCAQDMNHEYMSWYRQDPGMGLRLIHYSVAIQTDQGEVP NGYNVSRSTIEDFPLRLLSAAPSQTSVYFCASSYLGNTGELFFGEGSRLTVL |
| 73 | IL2 | Human IL2 | APTSSSTKKTQLQLEHLLLDLQMILNGINNYKNPKLTRMLTFKFYMPKKATELKHLQCLEEELK PLEEVLNLAQSKNFHLRPRDLISNINVIVLELKGSETTFMCEYADETATIVEFLNRWITFCQSIIS |
| | | | TLT |
| 74 | Anti-GFP VH | Nanobody™ | QVQLVESGGALVQPGGSLRLSCAASGFPVNRYSMRWYRQAPGKEREWVAGMSSAGDRSS YEDSVKGRFTISRDDARNTVYLQMNSLKPEDTAVYYCNVNVGFEYWGQGTQVTVSS |
| 75 | Anti-IL1R1 | DOM4-122-23 Vk | DIQMTQSPSSLSASVGDRVTITCRASQSIIKHLKWYQQKPGKAPKLLIYGASRLQSGVPSRFS GSGSGTDFTLTISSLQPEDFATYYCQQGARWPQTFGQGTKVEIKR |
| 76 | FLT1 | EYLEA™ | SDTGRPFVEMYSEIPEIIHMTEGRELVIPCRVTSPNITVTLKKFPLDTLIPDGKRIIWDS RKGFIISNATYKEIGLLTCEATVNGHLYKTNYLTHRQTNTIIDV |
| 77 | KDR | EYLEA™ | VLSPSHGIELSVGEKLVLNCTARTELNVGIDFNUVEYPSSKHQHKKLVNRDLKTQSGSEMKKFL STLTIDGVTRSDQGLYTCAASSGLMTKKNSTFVRVHEK |
| 78 | | Aflibercept | SDTGRPFVEMYSEIPEIIHMTEGRELVIPCRVTSPNITVTLKKFPLDTLIPDGKRIIWDS RKGFIISNATYKEIGLLTCEATVNGHLYKTNYLTHRQTNTIIDVVLSPSHGIELSVGEKL VLNCTARTELNVGIDFNWEYPSSKHQHKKLVNRDLKTQSGSEMKKFLSTLTIDGVTRSDQ GLYTCAASSGLMTKKNSTFVRVHEKDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPS DIAVEWESNGQPENNYKTTPPVLDSDGSFFLYSKLTVDKSRWQQGNVFSCSVMHEALHNH |
| | | | YTQKSLSLSPG |
| 79 | GLP-1(7-37)-Pro9 | | HAPGTFTSDVSSYLEGQAAKEFIAWLVKGRG |
| 80 | Peptide YY | | IKPEAPGEDASPEELNRYYASLRHYLNLVTRQRY |
| 81 | EXENDIN-4 | | HGEGTFTSDLSKQMEEEAVRLFIEWLKNGGPSSGAPPPS |
| 82 | | DURAGLUTIDE™ | HGEGTFTSDVSSYLEEQAAKEFIAWLVKGGGGGGGSGGGGSGGGGSAESKYGPPCPPCPA PEAAGGPSVFLFPPKPKDTLMISRTPEVTCVVVDVSQEDPEVQFNWYVDGVEVHNAKTKPRE EQFNSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDKSRW QEGNVFSCSVMHEALHNHYTQKSLSLSLG |

∗indicates product comprising the domain.

TABLE 5

Amino acid sequences of Quad polypeptides

| SEQ ID NO: | POLYPEPTIDE | AMINO ACID SEQUENCE |
|---|---|---|
| 83 | Quad 1 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVSTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV LTDREWAEEWKHLDHLLNCIMDMVEKTRRSLTVLRRCQEADREELNYWIRRYSDAE |
| 84 | Quad 2 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVSTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV GGGGSLTDREWAEEWKHLDHLLNCIMDMVEKTRRSLTVLRRCQEADREELNYWIRRYSDA E |
| 85 | Quad 3 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVCTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV LTDREWAEEWKHLDHLLNCIMDMVEKTRRSLTVLRRCQEADREELNYWIRRYSDAE |
| 86 | Quad 4 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVCTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV GGGGSLTDREWAEEWKHLDHLLNCIMDMVEKTRRSLTVLRRCQEADREELNYWIRRYSDA E |
| 87 | Quad 5 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVCTDPQPLKEQPALNDSRYALSSRLRVSATFWQDPRNHFRCQVQFYGLSENDE WTQDRAKPVTQIVSAEAWGRADLTDREWAEEWKHLDHLLNCIMDMVEKTRRSLTVLRRCQ EADREELNYWIRRYSDAE |
| 88 | Quad 6 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVCTDPQPLKEQPALNDSRYALSSRLRVSATFWQDPRNHFRCQVQFYGLSENDE WTQDRAKPVTQIVSAEAWGRADGGGGSLTDREWAEEWKHLDHLLNCIMDMVEKTRRSLTV |
| | | LRRCQEADREELNYWIRRYSDAE |
| 89 | Quad 7 | KQEVTQIPAALSVPEGENLVLNCSFTDSAIYNLQWFRQDPG KGLTSLLLITPWQREQTSGRLNASLDKSSGRSTLYIAASQPGDSATYLCAVRPLLDGTYI PTFGRGTSLIVHPYIQNPDPAVYQLRDSKSSDKSVCLFTDFDSQTNVSQSKDSDVYITDK TVLDMRSMDFKSNSAVAWSNKSDFACANAFNNSIIPEDTFFPSPESSTVAAPSVFIFPPS DEQLKSGTASVVCLLNNFYPREAKVQWKVDNALQSGNSQESVTEQDSKDSTYSLSSTLTL SKADYEKHKVYACEVTHQGLSSPVTKSFNRGEC |
| 90 | Quad 8 | KQEVTQIPAALSVPEGENLVLNCSFTDSAIYNLQWFRQDPG KGLTSLLLITPWQREQTSGRLNASLDKSSGRSTLYIAASQPGDSATYLCAVRPLLDGTYI PTFGRGTSLIVHPYIQNPDPAVYQLRDSKSSDKSVCLFTDFDSQTNVSQSKDSDVYITDK CVLDMRSMDFKSNSAVAWSNKSDFACANAFNNSIIPEDTFFPSPESSTVAAPSVFIFPPS DEQLKSGTASVVCLLNNFYPREAKVQWKVDNALQSGNSQESVTEQDSKDSTYSLSSTLTL SKADYEKHKVYACEVTHQGLSSPVTKSFNRGEC |
| 91 | Quad 9 | KQEVTQIPAALSVPEGENLVLNCSFTDSAIYNLQWFRQDPG KGLTSLLLITPWQREQTSGRLNASLDKSSGRSTLYIAASQPGDSATYLCAVRPLLDGTYI |
| | | PTFGRGTSLIVHPYIQNPDPAVYQLRDSKSSDKSVCLFTDFDSQTNVSQSKDSDVYITDK CVLDMRSMDFKSNSAVAWSNKSDFACANAFNNSIIPEDTFFPSPESS |
| 92 | Quad 10 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVSTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV LTDREWAEEWKHLDHLLNCIMDMVEKTRRSLTVLRRCQEADREELNYWIRRYSDAEAPTS SSTKKTQLQLEHLLLDLQMILNGINNYKNPKLTRMLTFKFYMPKKATELKHLQCLEEELK PLEEVLNLAQSKNFHLRPRDLISNINVIVLELKGSETTFMCEYADETATIVEFLNRWITF CQSIISTLT |
| 93 | Quad 11 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV GGGGSLTDREWAEEWKHLDHLLNCIMDMVEKTRRSLTVLRRCQEADREELNYWIRRYSDA EAPTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLN RWITFCQSIISTLT |
| 94 | Quad 12 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVSTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV EPKSCDKTHTCPPCPLTDREWAEEWKHLDHLLNCIMDMVEKTRRSLTVLRRCQEADREEL NYWIRRYSDAE |
| 95 | Quad 13 | NAGVTQTPKFQVLKTGQSMTLQCAQDMNHEYMSWYRQDPGM GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVSTDPQPLKEQPALNDSRYALSSRLRVSATFWQDPRNHFRCQVQFYGLSENDE WTQDRAKPVTQIVSAEAWGRADASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV EPKSCDKTHTCPPCPGGGGSLTDREWAEEWKHLDHLLNCIMDMVEKTRRSLTVLRRCQEA DREELNYWIRRYSDAE |
| 96 | Quad 14 | QVQLVESGGALVQPGGSLRLSCAASGFPVNRYSMRWYRQAP GKEREWVAGMSSAGDRSSYEDSVKGRFTISRDDARNTVYLQMNSLKPEDTAVYYCNVNVG FEYWGQGTQVTVSSKLTDREWAEEWKHLDHLLNCIMDMVEKTRRSLTVLRRCQEADREEL NYWIRRYSDAE |
| 97 | Quad 15 | QVQLVESGGALVQPGGSLRLSCAASGFPVNRYSMRWYRQAP GKEREWVAGMSSAGDRSSYEDSVKGRFTISRDDARNTVYLQMNSLKPEDTAVYYCNVNVG |
| | | FEYWGQGTQVTVSSKGGGGSLTDREWAEEWKHLDHLLNCIMDMVEKTRRSLTVLRRCQEA DREELNYWIRRYSDAE |
| 98 | Quad 18 | QVQLVESGGALVQPGGSLRLSCAASGFPVNRYSMRWYRQAP GKEREWVAGMSSAGDRSSYEDSVKGRFTISRDDARNTVYLQMNSLKPEDTAVYYCNVNVG FEYWGQGTQVTVSSKLTDREWAEEWKHLDHLLNCIMDMVEKTRRSLTVLRRCQEADREEL NYWIRRYSDAEAPTSSSTKKTQLQLEHLLLDLQMILNGINNYKNPKLTRMLTFKFYMPKK ATELKHLQCLEEELKPLEEVLNLAQSKNFHLRPRDLISNINVIVLELKGSETTFMCEYAD ETATIVEFLNRWITFCQSIISTLT |
| 99 | Quad 19 | QVQLVESGGALVQPGGSLRLSCAASGFPVNRYSMRWYRQAP GKEREWVAGMSSAGDRSSYEDSVKGRFTISRDDARNTVYLQMNSLKPEDTAVYYCNVNVG FEYWGQGTQVTVSSKGGGGSLTDREWAEEWKHLDHLLNCIMDMVEKTRRSLTVLRRCQEA DREELNYWIRRYSDAEAPTSSSTKKTQLQLEHLLLDLQMILNGINNYKNPKLTRMLTFKF YMPKKATELKHLQCLEEELKPLEEVLNLAQSKNFHLRPRDLISNINVIVLELKGSETTFM CEYADETATIVEFLNRWITFCQSIISTLT |
| 100 | Quad 20 | KQEVTQIPAALSVPEGENLVLNCSFTDSAIYNLQWFRQDPG |
| | | KGLTSLLLITPWQREQTSGRLNASLDKSSGRSTLYIAASQPGDSATYLCAVRPLLDGTYI PTFGRGTSLIVHPYIQNPDPAVYQLRDSKSSDKSVCLFTDFDSQTNVSQSKDSDVYITDK TVLDMRSMDFKSNSAVAWSNKSDFACANAFNNSIIPEDTFFPSPESSASTKGPSVFPLAP SSKSTSGGTAALGCLVKDYFPEPVTVSWNSGALTSGVHTFPAVLQSSGLYSLSSVVTVPS SSLGTQTYICNVNHKPSNTKVDKKVLHGTRQEEMIDHRLTDREWAEEWKHLDHLLNCIMD MVEKTRRSLTVLRRCQEADREELNYWIRRYSDAEDLK |
| 101 | Quad 21 | KQEVTQIPAALSVPEGENLVLNCSFTDSAIYNLQWFRQDPG KGLTSLLLITPWQREQTSGRLNASLDKSSGRSTLYIAASQPGDSATYLCAVRPLLDGTYI PTFGRGTSLIVHPYIQNPDPAVYQLRDSKSSDKSVCLFTDFDSQTNVSQSKDSDVYITDK TVLDMRSMDFKSNSAVAWSNKSDFACANAFNNSIIPEDTFFPSPESSASTKGPSVFPLAP SSKSTSGGTAALGCLVKDYFPEPVTVSWNSGALTSGVHTFPAVLQSSGLYSLSSVVTVPS SSLGTQTYICNVNHKPSNTKVDKKVGGGGSLHGTRQEEMIDHRLTDREWAEEWKHLDHLL NCIMDMVEKTRRSLTVLRRCQEADREELNYWIRRYSDAEDLK |
| 102 | Quad 22 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE LFFGEGSRLTVLEDLKNVFPPEVAVFEPSEAEISHTQKATLVCLATGFYPDHVELSWWVN GKEVHSGVSTDPQPLKEQPALNDSRYALSSRLRVSATFWQDPRNHFRCQVQFYGLSENDE WTQDRAKPVTQIVSAEAWGRADTVAAPSVFIFPPSDEQLKSGTASWCLLNNFYPREAKV QWKVDNALQSGNSQESVTEQDSKDSTYSLSSTLTLSKADYEKHKVYACEVTHQGLSSPVT KSFNRGEC |
| 103 | Quad 28 | EVQLVESGGGLVQPGRSLRLSCAASGFTFDDYAMHWVRQAP GKGLEWVSAITWNSGHIDYADSVEGRFTISRDNAKNSLYLQMNSLRAEDTAVYYCAKVSY LSTASSLDYWGQGTLVTVSSASTKGPSVFPLAPSSKSTSGGTAALGCLVKDYFPEPVTVS WNSGALTSGVHTFPAVLQSSGLYSLSSWTVPSSSLGTQTYICNVNHKPSNTKVDKKVLH GTRQEEMIDHRLTDREWAEEWKHLDHLLNCIMDMVEKTRRSLTVLRRCQEADREELNYWI RRYSDAEDLK |
| 104 | Quad 29 | EVQLVESGGGLVQPGRSLRLSCAASGFTFDDYAMHWVRQAP GKGLEWVSAITWNSGHIDYADSVEGRFTISRDNAKNSLYLQMNSLRAEDTAVYYCAKVSY LSTASSLDYWGQGTLVTVSSASTKGPSVFPLAPSSKSTSGGTAALGCLVKDYFPEPVTVS |
| | | WNSGALTSGVHTFPAVLQSSGLYSLSSWTVPSSSLGTQTYICNVNHKPSNTKVDKKVGG GGSLHGTRQEEMIDHRLTDREWAEEWKHLDHLLNCIMDMVEKTRRSLTVLRRCQEADREE LNYWIRRYSDAEDLK |
| 105 | Quad 30 | DIQMTQSPSSLSASVGDRVTITCRASQGIRNYLAWYQQ KPGKAPKLLIYAASTLQSGVPSRFSGSGSGTDFTLTISSLQPEDVATYYCQRYNRAPYTF GQGTKVEIKRTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSFNRGEC |
| 106 | Quad 31 | EVQLVESGGGLVQPGGSLRLSCAASGYTFTNYGMNWVRQAP GKGLEWVGWINTYTGEPTYAADFKRRFTFSLDTSKSTAYLQMNSLRAEDTAVYYCAKYPH YYGSSHWYFDVWGQGTLVTVSSASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV LHGTRQEEMIDHRLTDREWAEEWKHLDHLLNCIMDMVEKTRRSLTVLRRCQEADREELNY WIRRYSDAEDLK |
| 107 | Quad 32 | EVQLVESGGGLVQPGGSLRLSCAASGYTFTNYGMNWVRQAP GKGLEWVGWINTYTGEPTYAADFKRRFTFSLDTSKSTAYLQMNSLRAEDTAVYYCAKYPH |
| | | YYGSSHWYFDVWGQGTLVTVSSASTKGPSVFPLAPSSKSTSGGTAALGCLVKDYFPEPVT VSWNSGALTSGVHTFPAVLQSSGLYSLSSVVTVPSSSLGTQTYICNVNHKPSNTKVDKKV GGGGSLHGTRQEEMIDHRLTDREWAEEWKHLDHLLNCIMDMVEKTRRSLTVLRRCQEADR EELNYWIRRYSDAEDLK |
| 108 | Quad 33 | DIQMTQSPSSLSASVGDRVTITCSASQDISNYLNWYQQ KPGKAPKVLIYFTSSLHSGVPSRFSGSGSGTDFTLTISSLQPEDFATYYCQQYSTVPWTF GQGTKVEIKRTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSFNRGEC |
| 109 | Quad 34 | DIQMTQSPSSLSASVGDRVTITCRASQSIDSYLHWYQQK PGKAPKLLIYSASELQSGVPSRFSGSGSGTDFTLTISSLQPEDFATYYCQQVVWRPFTFG QGTKVEIKRGGGGSLHGTRQEEMIDHRLTDREWAEEWKHLDHLLNCIMDMVEKTRRSLTV LRRCQEADREELNYWIRRYSDAEDLK |
| 110 | Quad 36 | EVQLLESGGGLVQPGGSLRLSCAASGFTFAHETMVWVRQAPG KGLEWVSHIPPDGQDPFYADSVKGRFTISRDNSKNTLYLQMNSLRAEDTAVYHCALLPKR GPWFDYWGQGTLVTVSSGGGGSLHGTRQEEMIDHRLTDREWAEEWKHLDHLLNCIMDMVE |
| | | KTRRSLTVLRRCQEADREELNYWIRRYSDAEDLK |
| 111 | Quad 38 | DIQMTQSPSSLSASVGDRVTITCRASQWIGPELSWYQQK PGKAPKLLIYHTSILQSGVPSRFSGSGSGTDFTLTISSLQPEDFATYYCQQYMFQPRTFG QGTKVEIRGGGGSLHGTRQEEMIDHRLTDREWAEEWKHLDHLLNCIMDMVEKTRRSLTVL RRCQEADREELNYWIRRYSDAEDLK |
| 112 | Quad 40 | DIQMTQSPTSLSASVGDRVTITCRASQWIGNLLDWYQQK PGEAPKLLIYYASFLQSGVPSRFSGGGFGTDFTLTISSLQPEDFATYYCQQANPAPLTFG QGTKVEIKRGGGGSLHGTRQEEMIDHRLTDREWAEEWKHLDHLLNCIMDMVEKTRRSLTV LRRCQEADREELNYWIRRYSDAEDLK |
| 113 | Quad 42 | DIQMTQSPSSLSASVGDRVTITCRASQWIGPELSWYQQK PGKAPKLLIYHTSILQSGVPSRFSGSGSGTDFTLTISSLQPEDFATYYCQQYMFQPRTFG QGTKVEIRGGGGSLHGTRQEEMIDHRLTDREWAEEWKHLDHLLNCIMDMVEKTRRSLTVL RRCQEADREELNYWIRRYSDAEDLKGGGGSDIQMTQSPTSLSASVGDRVTITCRASQWIG NLLDWYQQKPGEAPKLLIYYASFLQSGVPSRFSGGGFGTDFTLTISSLQPEDFATYYCQQ ANPAPLTFGQGTKVEIKR |
| 114 | Quad 44 | DIQMTQSPSSLSASVGDRVTITCRASQNIDSRLSWYQQK PGKAPKLLIYRTSVLQSGVPSRFSGSGSGTDFTLTISSLQPEDFATYYCQQWDMFPLTFG QGTKVEIKRGGGGSLHGTRQEEMIDHRLTDREWAEEWKHLDHLLNCIMDMVEKTRRSLTV LRRCQEADREELNYWIRRYSDAEDLK |
| 115 | Quad 46 | DIQMTQSPSSLSASVGDRVTITCRASQNIDSRLSWYQQK PGKAPKLLIYRTSVLQSGVPSRFSGSGSGTDFTLTISSLQPEDFATYYCQQWDMFPLTFG QGTKVEIKRGGGGSLHGTRQEEMIDHRLTDREWAEEWKHLDHLLNCIMDMVEKTRRSLTV LRRCQEADREELNYWIRRYSDAEDLKGGGGSDIQMTQSPSSLSASVGDRVTITCRASQDI GDRLRWYQQKPGKAPKLLIYHGSRLESGVPSRFSGSRSGTDFTLTISSLQPEDFATYYCQ QQWFRPYTFGQGTKVEIKR |

TABLE 6

DNA and amino acid sequences of NHR2 TDs

| SEQ ID NO: | NHR2 TD | DNA SEQUENCE |
|---|---|---|
| 116 | NHR2 TD | CTAACAGACAGAGAATGGGCAGAAGAGTGGAAACATCTTGACCATCTGTTAAACTGCATA ATGGACATGGTAGAAAAAACAAGGCGATCTCTCACCGTACTAAGGCGGTGTCAAGAAGCA GACCGGGAAGAATTGAATTACTGGATCCGGCGGTACAGTGACGCCGAG |
| 117 | NHR2^{∗} TD | TTGCATGGCACACGTCAAGAAGAAATGATTGATCACAGACTAACAGACAGAGAATGGGCA GAAGAGTGGAAACATCTTGACCATCTGTTAAACTGCATAATGGACATGGTAGAAAAAACA AGGCGATCTCTCACCGTACTAAGGCGGTGTCAAGAAGCAGACCGGGAAGAATTGAATTAC TGGATCCGGCGGTACAGTGACGCCGAG |
| 118 | NHR2^{∗∗} TD | GGCACACGTCAAGAAGAAATGATTGATCACAGACTAACAGACAGAGAATGGGCAGAAGAG TGGAAACATCTTGACCATCTGTTAAACTGCATAATGGACATGGTAGAAAAAACAAGGCGA TCTCTCACCGTACTAAGGCGGTGTCAAGAAGCAGACCGGGAAGAATTGAATTACTGGATC CGGCGGTACAGTGACGCCGAG |
| 119 | NHR2^{∗∗∗} TD | CAAGAAGAAATGATTGATCACAGACTAACAGACAGAGAATGGGCAGAAGAGTGGAAACAT CTTGACCATCTGTTAAACTGCATAATGGACATGGTAGAAAAAACAAGGCGATCTCTCACC GTACTAAGGCGGTGTCAAGAA |
| | NHR2 TD | AMINO ACID SEQUENCE |
| 120 | NHR2 TD | LTDREWAEEWKHLDHLLNCIMDMVEKTRRSLTVLRRCQEADREELNYWIRRYSDAE |
| 121 | NHR2^{∗} TD | LHGTRQEEMIDHRLTDREWAEEWKHLDHLLNCIMDMVEKTRRSLTVLRRCQEADREELNY WIRRYSDAEDLK |
| 122 | NHR2^{∗∗} TD | GTRQEEMIDHRLTDREWAEEWKHLDHLLNCIMDMVEKTRRSLTVLRRCQEADREELNYWI RRYSDAE |
| 123 | NHR2^{∗∗∗} TD | QEEMIDHRLTDREWAEEWKHLDHLLNCIMDMVEKTRRSLTVLRRCQE |

NHR2^{∗} TD and NHR2^{∗∗∗} TD include additional amino acid residues at the N- and/or C-terminus. NHR2^{∗∗∗} only includes amino acid residues of the annotated NHR2 domain according to Pubmed (Reference: UniProtKB - Q06455 (MTG8_HUMAN)

TABLE 7

Human p53 TD sequences

| SEQ ID NO: | p53 TD | DNA SEQUENCE |
|---|---|---|
| 124 | p53 TD | AAGAAGAAACCACTGGATGGAGAATATTTCACCCTTCAGATCCGTGGGCGTGAGCGCTTCGAG ATGTTCCGAGAGCTGAATGAGGCCTTGGAACTCAAGGATGCCCAGGCTGGGAAGGAGCCAGG G |
| 125 | p53^{∗} TD | GGAGAATATTTCACCCTTCAGATCCGTGGGCGTGAGCGCTTCGAGATGTTCCGAGAGCTGAAT GAGGCCTTGGAACTCAAGGATGCCCAGGCTGGG |
| SEQ ID NO: | p53 TD | AMINO ACID SEQUENCE |
| 126 | p53 TD | KKKPLDGEYFTLQIRGRERFEMFRELNEALELKDAQAGKEPG |
| 10 | p53^{∗} TD | GEYFTLQIRGRERFEMFRELNEALELKDAQAG |

p53^{∗} TD is a truncated version of p53 TD

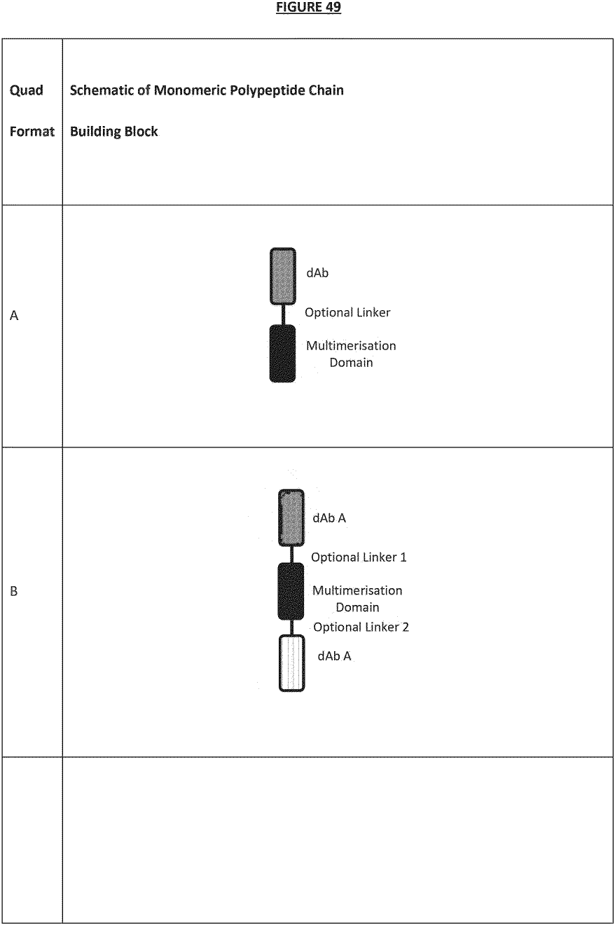

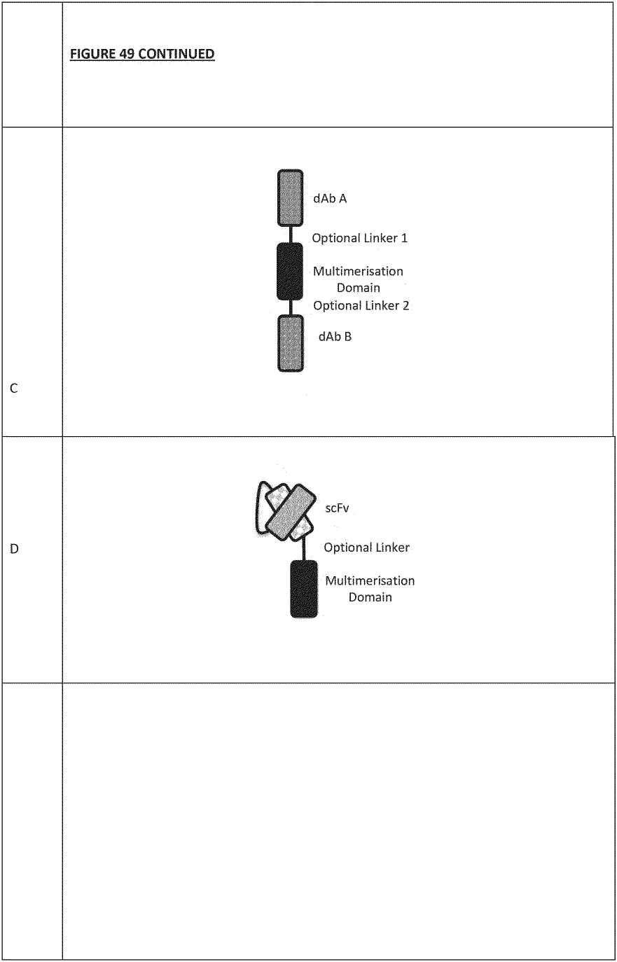

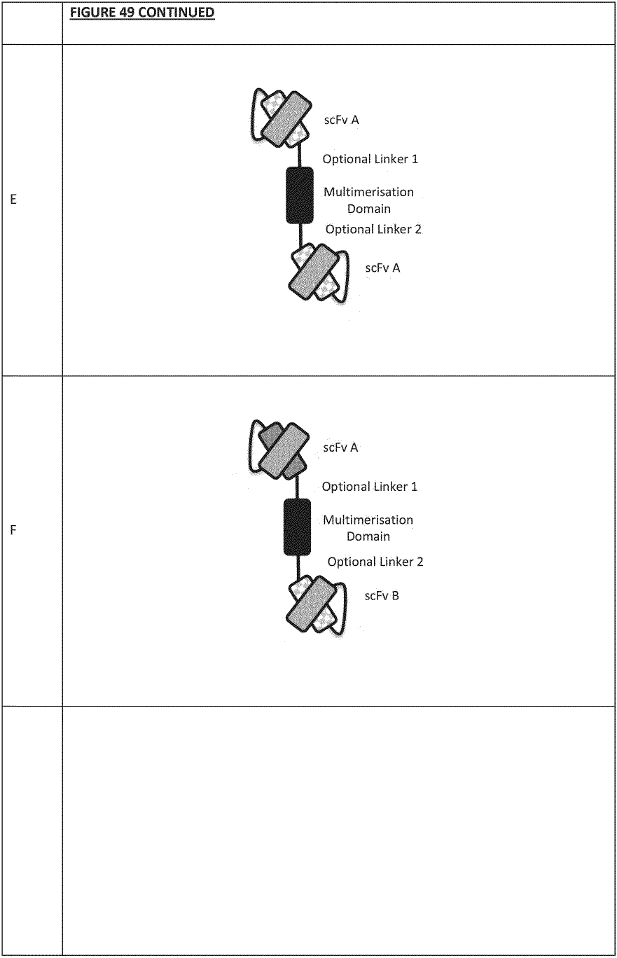

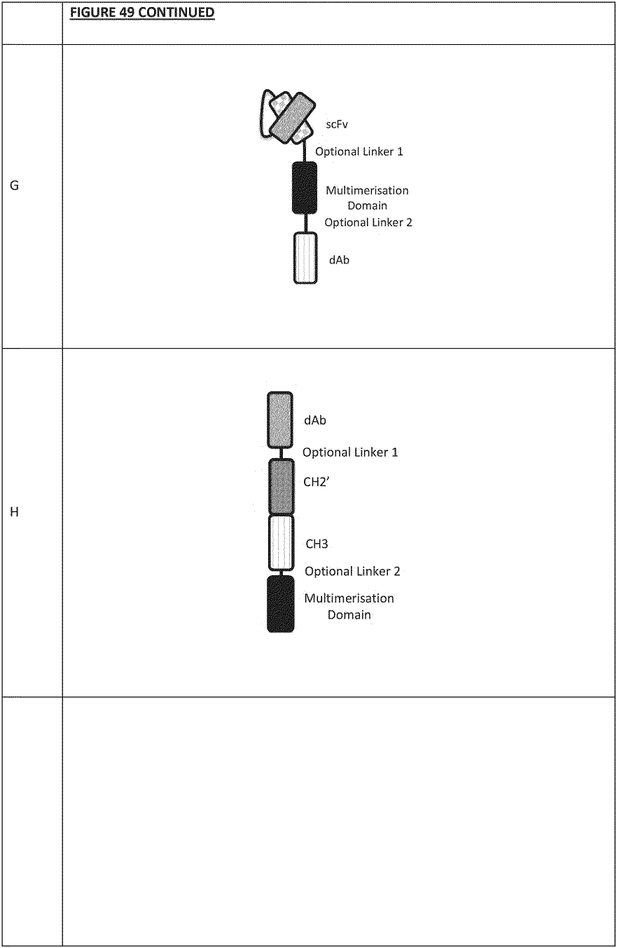

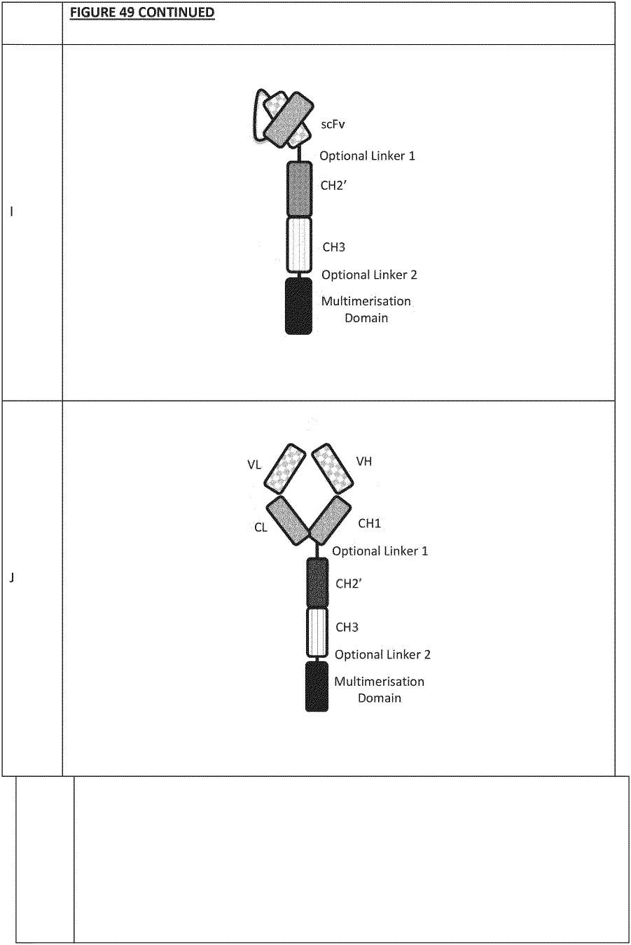

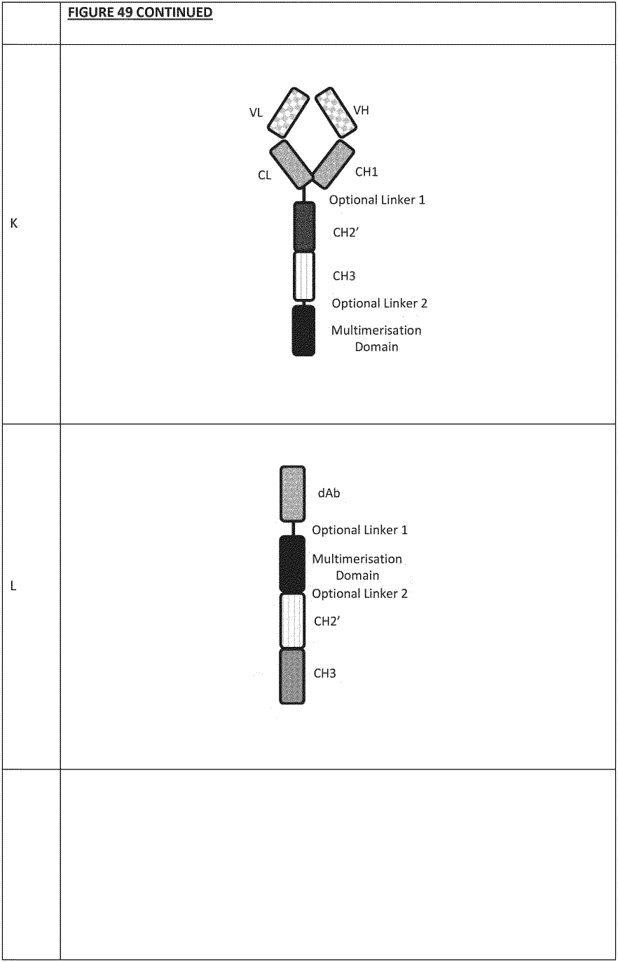

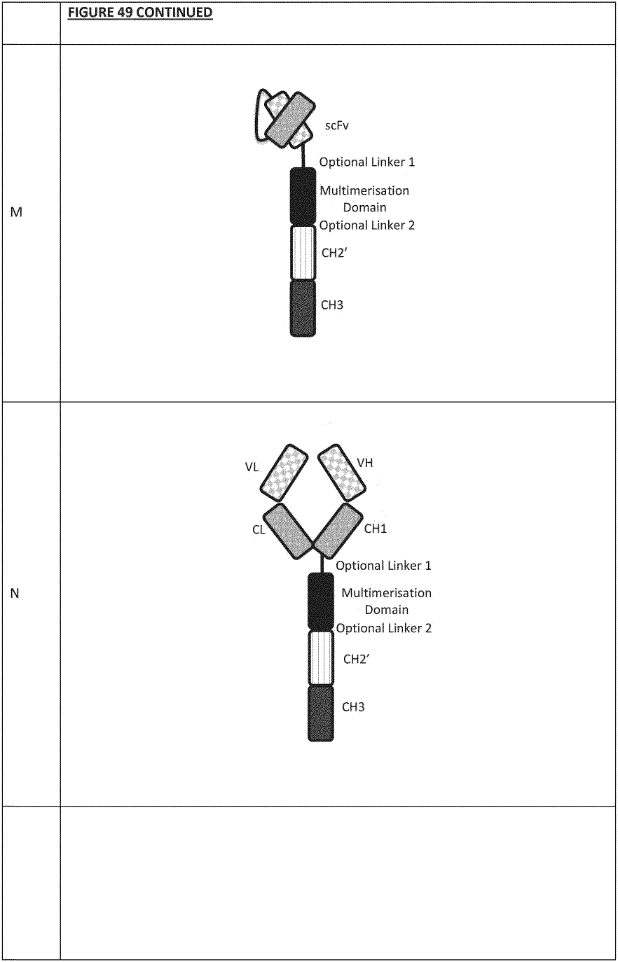

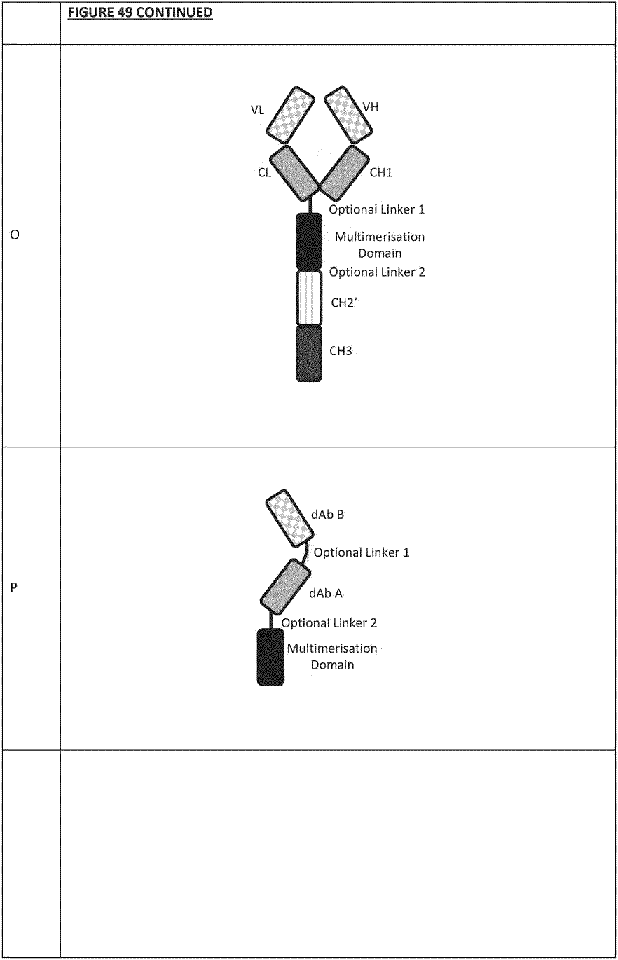

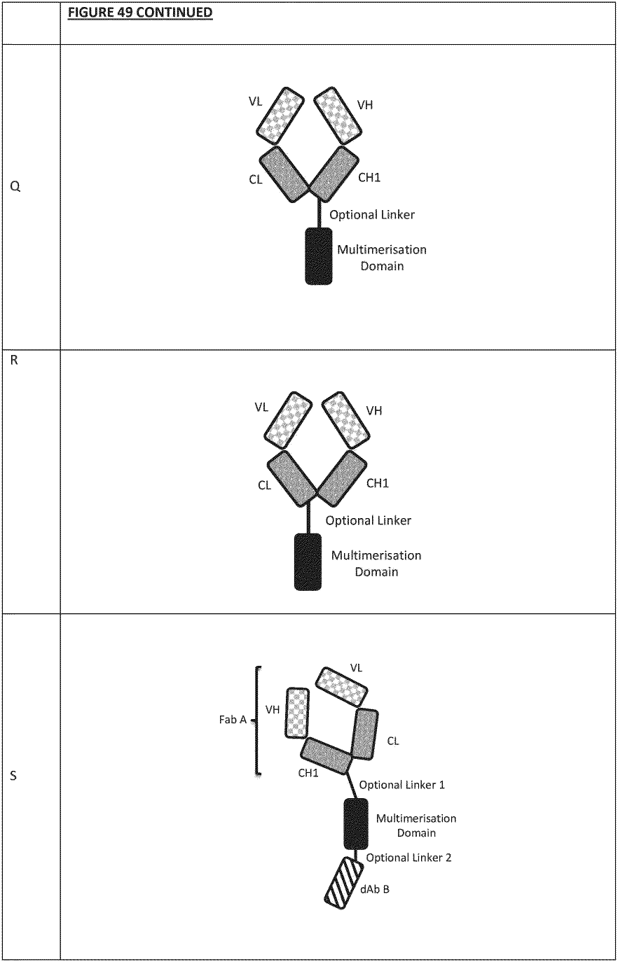

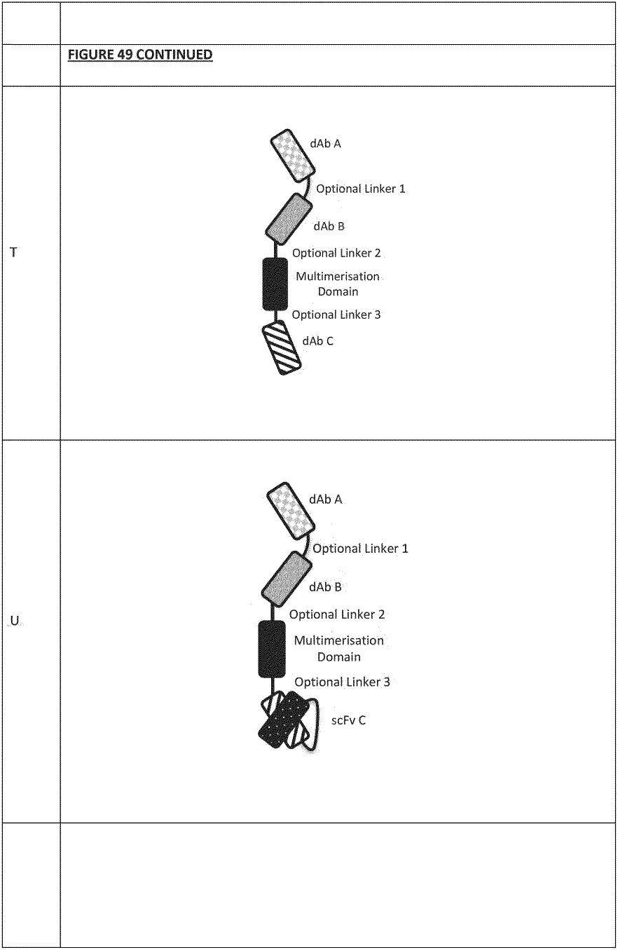

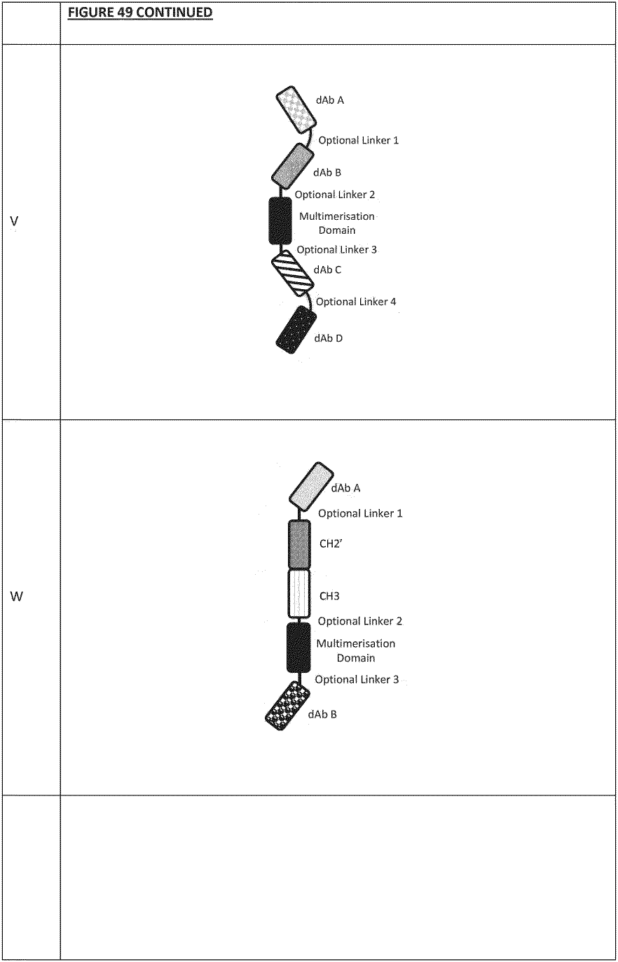

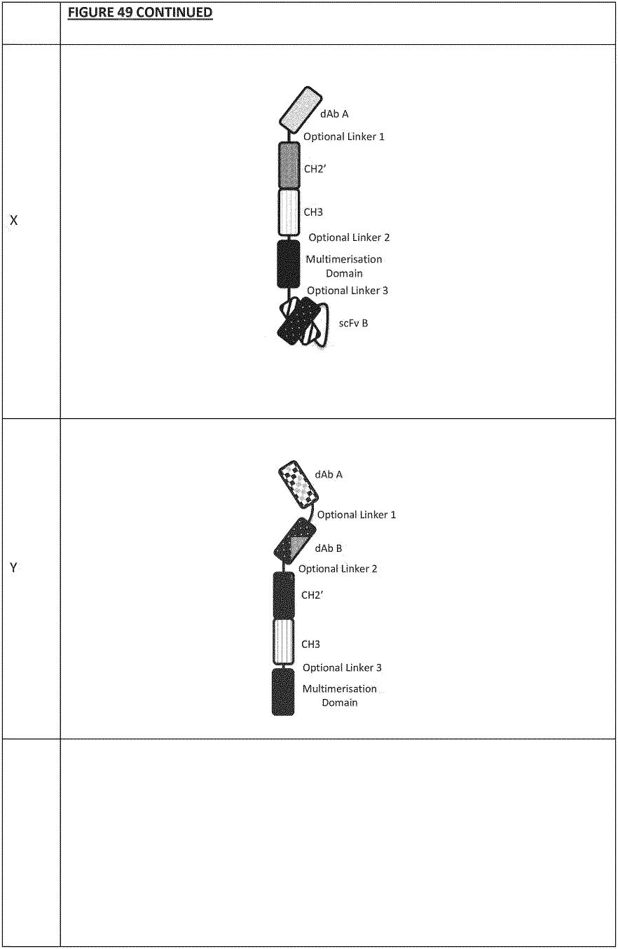

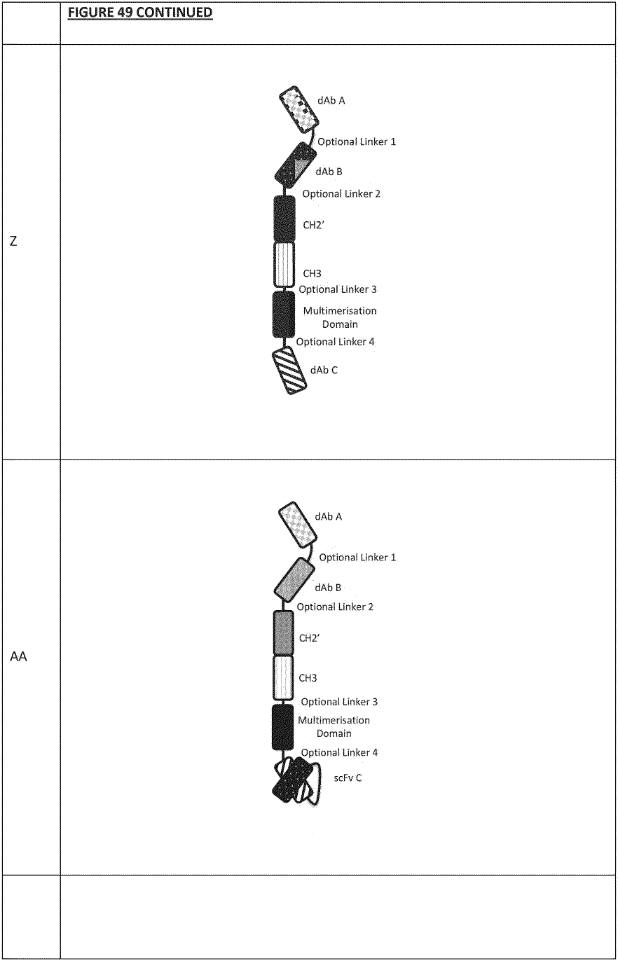

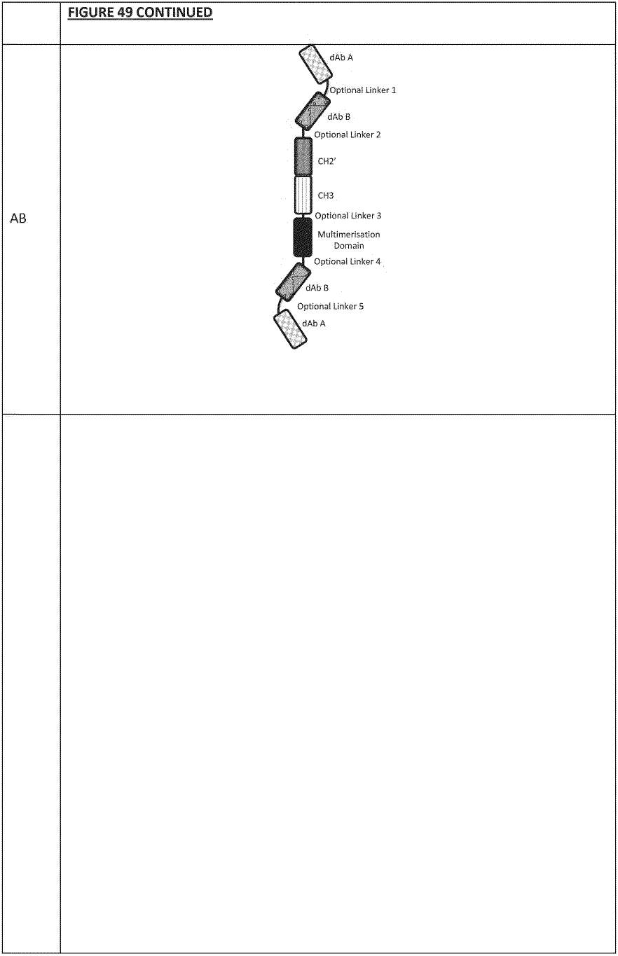

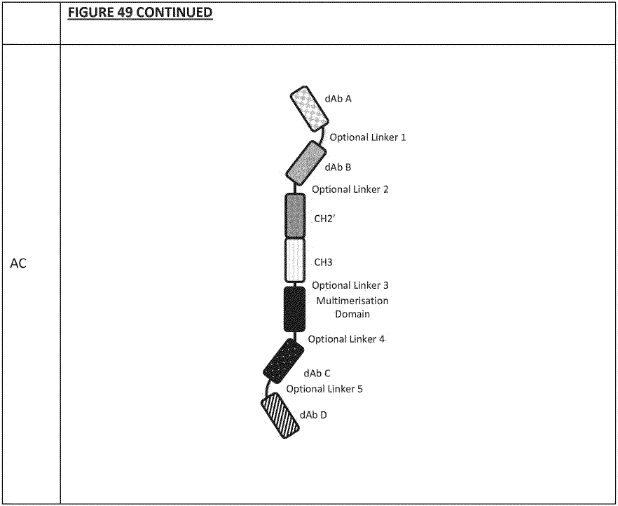

Table 8: Description of monomeric building block of Quad Formats A - AC and as outlined in FIG. 49

In the schematics, components of polypeptide chains are N- to C-terminally from top to the bottom of each chain as shown.

Herein the terms “SAM”, “self-associating multimerization domain” and “multimerisation domain” are used interchangeably. A SAM may, for example be called a TD herein. A TD may be a tetradimerisation domain as disclosed herein, eg, a p53, p63, p73 or NHR2 domain or homologue or orthologue thereof.

Valency indicates binding site number in the tetramer form when using a TD as a SAM.

| Quad Format (See FIG. 49 ) | Description |
|---|---|
| A | Tetravalent dAb Quad |
| | Monomeric building block of Quad format ‘A’ contains self-assembling multimerisation (SAM) domain linked to a dAb (an antibody single variable domain). The dAb can be linked at either the N- or C-terminus of the SAM domain via an optional peptide linker. The dAb binding domain can either be a VH, Vκ or Vλ. |
| B | Octavalent Monospecific dAb Quad |
| | Monomeric building block of Quad format ‘B’ contains SAM domain linked to dAb binding domains at both N- and C-terminus via optional peptide linkers 1 and/or 2. dAb binding domains can either be a VH, Vκ or Vλ. The dAbs have the same antigen specificity. |
| C | Octavalent Bispecific dAb Quad |
| | Monomeric building block of Quad format ‘C’ contains SAM domain linked to two different dAb binding domains (dAb A and dAb B) at the N- and C-terminus via optional peptide linkers 1 and/or 2. The dAb binding domains can either be a VH, Vκ or Vλ. dAb A has an antigen specificity that is different from the antigen specificity of dAbB. |
| D | Tetravalent Monospecific scFv Quad |
| | Monomeric building block of Quad format ‘D’ contains SAM domain linked to an scFv binding domain. The scFv binding domain can be linked at either the N- or C-terminus of the SAM domain via an optional peptide linker. |
| E | Octavalent Monospecific scFv Quad |
| | Monomeric building block of Quad format ‘E’ contains SAM domain linked to the same type of scFv binding domains at both N- and C-terminus via optional peptide linkers 1 and/or 2. |
| F | Octavalent Bispecific scFv Quad |
| | Monomeric building block of Quad format ‘F’ contains SAM domain linked to two different scFv binding domains (scFv A and scFv B) at the N- and C-terminus via optional peptide linkers 1 and/or 2. scFv A has an antigen specificity that is different from the antigen specificity of scFv B. |
| G | Octavalent Bispecific scFv x dAb Quad |
| | Monomeric building block of Quad format ‘G’ contains SAM domain linked to two different binding domain formats (i.e. scFv and a dAb). The scFv and dAb binding domains can be fused to SAM domain via the N- and C-terminus, respectively or via C-and N-terminus, respectively. Both scFv and dAb binding domains are fused to SAM domain via optional peptide linkers 1 and/or 2. |
| H | Tetravalent Monospecific Ig-dAb Quad v1 |
| | Monomeric building block of Quad format ‘H’ contains SAM domain linked to Ig Fc region (eg, an IgG1 Fc region) comprising of CH3 and CH2 (eg, CH2′) domains in addition to a dAb. dAb binding domain can be linked to N- or C-terminus of the Fc region via an optional peptide linker 1 or 2, respectively. Similarly SAM domain can be linked to N- or C-terminus of the Fc region via an optional peptide linker 1 or 2, respectively. CH2′ domain is devoid of core hinge region. The dAb binding domain can either be a VH, Vκ or Vλ. “Monomeric Ig” indicates that the Fc (such as comprising a CH2′) does not directly pair with another Fc. |
| I | Tetravalent Monospecific Ig-scFv Quad v1 |
| | Monomeric building block of Quad format′I′ contains SAM domain linked to Ig Fc region consisting of CH3 and CH2 (eg, CH2′) domains in addition to a scFv binding domain. scFv binding domain can be linked to N- or C-terminus of the Fc region via an optional peptide linker 1 or 2, respectively. Similarly SAM domain can be linked to Nor C-terminus of the Fc region via an optional peptide linker 1 or 2, respectively. CH2′ domain is devoid of core hinge region or a CXXC core region motif. |
| J | Tetravalent Monospecific Ig-Fab Quad v1 |
| | Monomeric building block of Quad format ‘J’ comprises or consists of two chains. The chain containing SAM domain is linked to Ig Fc region comprising CH3 and CH2 (eg, CH2′) domains in addition to Ig Fab connected via the CH1 domain. Ig Fab is linked to the N-terminus of the Fc region via an optional peptide linker 1. The SAM domain is linked to C-terminus of the Fc region via an optional peptide linker 2. CH2′ domain is devoid of core hinge region. The second chain is an Ig light chain comprising of CL and VL domains. |
| K | Tetravalent Monospecific Ig-Fab Quad v2 |
| | Monomeric building block of Quad format ‘K’ comprises or consists of two chains. The chain containing SAM domain is linked to Ig Fc region comprising CH3 and CH2 (eg, CH2′) domains in addition to Ig Fab connected via the CL domain. Ig Fab is linked to the N-terminus of the Fc region via an optional peptide linker 1. The SAM domain is linked to C-terminus of the Fc region via an optional peptide linker 2. CH2′ domain is devoid of core hinge region. The second chain is an Ig heavy chain comprising CH1 and VH domains. |
| L | Tetravalent Monospecific Ig-dAb Quad v2 |
| | Monomeric building block of Quad format ‘L’ contains SAM domain linked to Ig Fc region consisting of CH3 and CH2 (eg, CH2′) domains in addition to a dAb antigen binding domain. dAb is linked to the SAM domain with an optional peptide linker 1. The Fc region is linked to the SAM domain via the C-terminus with an optional linker 2. CH2′ domain is devoid of core hinge region. The dAb can either be a VH, Vκ or Vλ. |
| M | Tetravalent Monospecific Ig-scFv Quad v2 |
| | Monomeric building block of Quad format ‘M’ contains SAM domain linked to Ig Fc region consisting of CH3 and CH2 (eg, CH2′) domains in addition to a scFv antigen binding domain. scFv is linked to the SAM domain via the N-terminus with an optional peptide linker 1. The Fc region is linked to the SAM domain via the C-terminus with an optional linker 2. CH2′ domain is devoid of core hinge region. |
| N | Tetravalent Monospecific Ig-Fab Quad v3 |
| | Monomeric building block of Quad format ‘N’ comprises or consists of two chains. The chain containing SAM domain is linked to Ig Fc region comprising CH3 and CH2 (eg, CH2′) domains in addition to Ig Fab connected via the CH1 domain. The Ig Fab is linked to SAM domain via the N-terminus with an optional peptide linker 1. The Ig Fc region is linked to SAM domain via the C-terminus with an optional peptide linker 2. The second chain is an Ig light chain consisting of CL and VL domains. |
| O | Tetravalent Monospecific Ig-Fab Quad v4 |
| | Monomeric building block of Quad format ‘O’ comprises or consists of two chains. The chain containing SAM domain is linked to Ig Fc region consisting of CH3 and CH2 (eg, CH2′) domains in addition to Ig Fab connected via the CL domain. The Ig Fab is linked to SAM domain via the N-terminus with an optional peptide linker 1. The Ig Fc region is linked to SAM domain via the C-terminus with an optional peptide linker 2. The second chain is an Ig heavy chain consisting of CH1 and VH domains. |
| P | Octavalent Bispecific Tandem dAb Quad |
| | Monomeric building block of Quad format ‘P’ contains SAM domain linked to two different dAbs (dAb A and dAb B) connected in tandem. The dAbs are connected together via an optional peptide linker 1 and the tandem dAbs are connected to SAM domain at either the N- or C-terminus via an optional peptide linker 2. dAb binding domains can either be a VH, Vκ or Vλ,. |
| Q | Tetravalent Fab Quad v1 |
| | Monomeric building block of Quad format ‘Q’ comprises or consists of two chains. The chain containing SAM domain is linked to Ig Fab via CH1 domain. Ig Fab is linked to the N-terminus of the SAM domain via an optional peptide linker. The second chain is an Ig light chain comprising CL and VL domains. |
| R | Tetravalent Fab Quad v2 |
| | Monomeric building block of Quad format ‘R’ comprises or consists of two chains. The chain containing SAM domain is linked to Ig Fab via CL domain. Ig Fab is linked to the N-terminus of the SAM domain via an optional peptide linker. The second chain is an Ig heavy chain comprising of CH1 and VH domains. |
| S | Octavalent Bispecific Fab x dAb Quad |
| | Monomeric building block of Quad format ‘S’ comprises or consists of two chains. The chain containing SAM domain is linked to different binding domains. Binding domain ‘A’ is a Fab linked to SAM domain at the N-terminus via an optional peptide linker 1. Binding domain ‘B’ is a dAb linked to the SAM domain at the C-terminus via an optional linker 2. The dAb binding domain can either be a VH, Vκ or Vλ. The second chain is an Ig light chain comprising CL and VL domains. The Fab binding domain ‘A’ could also be optionally linked via the CL of the light chain. |
| T | 12-Valent Trispecific dAb Quad |
| | Monomeric building block of Quad format’T′ contains SAM domain linked to three different dAbs (dAbs A, B & C) with specificity for either three different epitopes of the same target protein or a respective epitope on three different target proteins. The tandem dAbs ‘A’ & ‘B’ is linked to the SAM domain at the N-terminus via an optional linker 1 where dAbs ‘A’ & ‘B’ are linked together via an optional linker 2. A single dAb (dAb ‘C’) is linked at the C-terminus of the SAM domain via an optional linker 3. The dAb binding domains can either be a VH, Vκ or Vλ. In an alternative, the configuration (in N- to C-terminal direction) is dAb A - optional linker 1 - SAM optional linker 2 - dAb B - optional linker 3 - dAb C. |
| U | 12-Valent Trispecific dAb x scFv Quad |
| | Monomeric building block of Quad format ‘U’ contains SAM domain linked to three different binding domains (Tandem dAb containing dAbs A & B and an scFv ‘C’) with specificity for either three different epitopes of the same target protein or a respective epitope on three different target proteins. The tandem dAbs ‘A’ & ‘B’ is linked to the SAM domain at the N-terminus via an optional linker 1 where dAbs ‘A’ & ‘B’ are linked together via an optional linker 2. An scFv ‘C’ is linked at the C-terminus of the SAM domain via an optional linker 3. The dAb binding domains can either be a VH, Vκ or Vλ. In an alternative, the configuration (in N- to C-terminal direction) is (i) dAb A - optional linker 1 - SAM optional linker 2 - dAb B - optional linker 3 - scFv C; (ii) scFv- optional linker 1 - SAM optional linker 2 - dAb A - optional linker 3 - dAb B; or (iii) scFv- optional linker 1 - dAb A -optional linker 2 - SAM - optional linker 3 - dAb B. |
| V | 16-Valent Tetraspecific dAb Quad |
| | Monomeric building block of Quad format ‘V’ contains SAM domain linked to two tandem |
| | dAbs (consisting of dAbs A-D) with specificity for either four different epitopes of the same target protein or a respective epitope on four different target proteins. Each tandem dAb comprises two different dAbs linked together via one or more optional peptide linkers (Linker 1 and 4). The two tandem dAbs are linked to the SAM domain via the N- and C-terminus via optional peptide linkers 2 and 3 respectively. The dAb binding domains can either be a VH, Vκ or Vλ. In an alternative, all dAbs are the same and have the same epitope specificity. |
| W | Octavalent Bispecific Monomeric Ig-dAb Quad |
| | Monomeric building block of Quad format ‘W’ contains SAM domain linked to Ig Fc region comprising or consisting of CH3 and (CH2 (eg, CH2′) domains plus two different dAb binding domains. The Ig Fc region is linked to the SAM domain at the N-terminus via an optional peptide linker 2. The first dAb binding domain ‘A’ is linked to the Ig Fc region at the N-terminus via an optional peptide linker 1. The second dAb binding domain ‘B’ is linked to the SAM domain at the C-terminus via an optional peptide linker 3. The CH2′ domain is devoid of core hinge region. The dAb binding domains can either be a VH, Vκ or Vλ. In an alternative, the dAbs have the same antigen binding specificity. |
| X | Octavalent Bispecific Monomeric Ig-dAb x scFv Quad |
| | Monomeric building block of Quad format ‘X’ contains SAM domain linked to Ig Fc region comprising or consisting ofcCH3 and CH2 (eg, CH2′) domains plus two different binding domains. The Ig Fc region is linked to the SAM domain at the N-terminus via an optional peptide linker 2. The first binding domain (dAb A′) is linked to the Ig Fc region at the N-terminus via an optional peptide linker 1. The second binding domain (scFv ‘B’) is linked to the SAM domain at the C-terminus via an optional peptide linker 3. The CH2′ domain is devoid of core hinge region. The dAb binding domain can either be a VH, Vκ or Vλ. In an alternative, the dAb and scFv have the same antigen binding specificity. |
| Y | Octavalent Bispecific Monomeric Ig-Tandem dAb Quad |
| | Monomeric building block of Quad format ‘Y’ contains SAM domain linked to Ig Fc region comprising or consisting of CH3 and CH2 (eg, CH2′) domains plus a tandem dAb containing dAbs ‘A’ & ‘B’ linked together via an optional peptide linker 1. The Ig Fc region is linked to the SAM domain at the N-terminus via an optional peptide linker 3. The tandem dAb is linked to the Ig Fc region at the N-terminus via an optional peptide linker 2. The CH2′ domain is devoid |
| | of core hinge region. The dAb binding domains can either be a VH, Vκ or Vλ. In an alternative, the dAbs have the same antigen binding specificity. |
| Z | 12-Valent Trispecific Monomeric Ig-dAb Quad |
| | Monomeric building block of Quad format ‘Z’ contains SAM domain linked to Ig Fc region comprising or consisting of CH3 and CH2 (eg, CH2′) domains plus a tandem dAb containing dAbs ‘A’ & ‘B’ linked together via an optional peptide linker 1 and a single dAb binding domain ‘C’. The Ig Fc region is linked to the SAM domain at the N-terminus via an optional peptide linker 3. The tandem dAb is linked to the Ig Fc region at the N-terminus via an optional peptide linker 2. The dAb binding domain ‘C’ is linked to the SAM domain at the C-terminus via an optional peptide linker 4. The CH2′ domain is devoid of core hinge region. The dAb binding domains can either be a VH, Vκ or Vλ. In an alternative, the dAbs have the same antigen binding specificity. |
| AA | 12-Valent Trispecific Monomeric Ig-dAb x scFv Quad |
| | Monomeric building block of Quad format ‘AA’ contains SAM domain linked to Ig Fc region comprising or consisting of CH3 and CH2′ domains plus a tandem dAb containing dAbs ‘A’ & ‘B’ linked together via an optional peptide linker 1 and a scFv containing binding domain ‘C’. The Ig Fc region is linked to the SAM domain at the N-terminus via an optional peptide linker 3. The tandem dAb is linked to the Ig Fc region at the N-terminus via an optional peptide linker 2. The scFv binding domain ‘C’ is linked to the SAM domain at the C-terminus via an optional peptide linker 4. The CH2′ domain is devoid of core hinge region. The dAb binding domains can either be a VH, Vκ or Vλ. In an alternative, the dAbs and scFv have the same antigen binding specificity. |
| AB | 16-Valent Bispecific Monomeric Ig-Tandem dAb Quad |
| | Monomeric building block of Quad format ‘AB’ contains SAM domain linked to Ig Fc region comprising or consisting of CH3 and CH2 (eg, CH2′) domains plus two identical tandem |
| | dAbs containing dAbs ‘A’ & ‘B’ linked together via optional peptide linkers 1 and 5, respectively. The Ig Fc region is linked to the SAM domain at the N-terminus via an optional peptide linker 3. The first tandem dAb is linked to the Ig Fc region at the N-terminus via an optional peptide linker 2. The second tandem dAb is linked to the SAM domain at the C-terminus via an optional peptide linker 4. The CH2′ domain is devoid of core hinge region. The dAb binding domains can either be a VH, Vκ or Vλ. In an alternative, the dAbs have the same antigen binding specificity. |
| AC | 16-Valent Tetraspecific Monomeric Ig-Tandem dAb Quad |
| | Monomeric building block of Quad format ‘AC’ contains SAM domain linked to Ig Fc region comprising or consisting of CH3 and CH2 (eg, CH2′) domains plus two different tandem dAbs containing dAbs ‘A’ & ‘B’ and dAbs ‘C’ & ‘D’ linked together via optional peptide linkers 1 and 5 respectively. The Ig Fc region is linked to the SAM domain at the N-terminus via an optional peptide linker 3. The first tandem dAb containing dAbs ‘A’ & ‘B’ is linked to the Ig Fc region at the N-terminus via an optional peptide linker 2. The second tandem dAb containing dAbs ‘C’ & ‘D’ is linked to the SAM domain at the C-terminus via an optional peptide linker 4. The CH2′ domain is devoid of core hinge region. The dAb binding domains can either be a VH, Vκ or Vλ. In an alternative, the dAbs AB or CD or AC or AD or BC or BD have the same antigen binding specificity. For example AD have a first antigen specificity and BC have a second antigen specificity. For example AB have a first antigen specificity and CD have a second antigen specificity |

TABLE 9

DNA sequences encoding Quad polypeptides

| | | |
|---|---|---|
| SEQ ID NO. | QUAD NO. | NUCLEOTIDE SEQUENCE |
| 139 | Quad 51 | ATGGGATGGTCTTGTATAATTCTGTTCCTGGTGGCAACAGCAA CAGGAGTGCATAGCGAGGTGCAGCTGGTGGAGTCTGGGGGA GGCTTGGTACAGCCCGGCAGGTCCCTGAGACTCTCCTGTGCGG CCTCTGGATTCACCTTTGATGATTATGCCATGCACTGGGTCCGG CAAGCTCCAGGGAAGGGCCTGGAATGGGTCTCAGCTATCACTT GGAATAGTGGTCACATAGACTATGCGGACTCTGTGGAGGGCC GATTCACCATCTCCAGAGACAACGCCAAGAACTCCCTGTATCTG CAAATGAACAGTCTGAGAGCTGAGGATACGGCCGTATATTACT GTGCGAAAGTCTCGTACCTTAGCACCGCGTCCTCCCTTGACTAT TGGGGCCAAGGTACCCTGGTCACCGTCTCGAGTGGCGGCGGA GGCAGCGGAGGCGGAGGTTCTGGAGGAGGGGGGAGTGACAT CCAGATGACCCAGTCTCCATCCTCCCTGTCTGCATCTGTAGGGG ACAGAGTCACCATCACTTGTCGGGCAAGTCAGGGCATCAGAAA TTACTTAGCCTGGTATCAGCAAAAACCAGGGAAAGCCCCTAAG CTCCTGATCTATGCTGCATCCACTTTGCAATCAGGGGTCCCATC TCGGTTCAGTGGCAGTGGATCTGGGACAGATTTCACTCTCACC ATCAGCAGCCTACAGCCTGAAGATGTTGCAACTTATTACTGTCA AAGGTATAACCGTGCACCGTATACTTTTGGCCAGGGGACCAAG GTGGAAATCAAAAAGAAGAAACCACTGGATGGAGAATATTTC ACCCTTCAGATCCGTGGGCGTGAGCGCTTCGAGATGTTCCGAG AGCTGAATGAGGCCTTGGAACTCAAGGATGCCCAGGCTGGGA AGGAGCCAGGG |
| 140 | Quad 53 Mon | ATGGGCTGGTCCTGCATCATCCTGTTTCTGGTGGCCACAGCCA CAGGCGTGCACAGCGATATTGTGCTGACACAGAGCCCCGCCAT CCTGAGTGCTTCTCCAGGCGAGAAAGTGACCATGACCTGCAGA GCCAGCAGCAGCGTGAACTACATGGACTGGTATCAGAAGAAG CCCGGCAGCAGCCCCAAGCCTTGGATCTACGCCACAAGCAATC TGGCCAGCGGAGTGCCTGCCAGATTTTCTGGCTCTGGCAGCGG CACAAGCTACAGCCTGACAATCAGCAGAGTGGAAGCCGAGGA TGCCGCCACCTACTACTGTCAGCAGTGGTCCTTCAATCCTCCTA |
| | | CCTTCGGCGGAGGCACCAAGCTGGAAATCAAGGGCTCTACATC TGGCGGAGGTGGAAGCGGAGGCGGAGGATCTGGTGGTGGTG GATCTTCTGAGGTCCAGCTGCAACAGTCTGGCGCCGAGCTTGT GAAACCTGGCGCCTCTGTGAAGATGAGCTGCAAGGCCAGCGG CTACACCTTCACCAGCTACAACATGCACTGGGTCAAGCAGACC CCTGGACAGGGACTCGAGTGGATCGGAGCCATCTATCCCGGC AATGGCGACACCTCCTACAACCAGAAGTTCAAGGGCAAAGCCA CACTGACCGCCGACAAGAGCAGCAGCACAGCCTACATGCAGCT GAGCAGCCTGACCAGCGAGGACAGCGCCGATTACTACTGCGC CAGAAGCAACTACTACGGCAGCTCCTACTGGTTCTTCGACGTG TGGGGAGCCGGCACCACAGTGACAGTGTCCAGC |
| 141 | Quad 53 Tet | ATGGGCTGGTCCTGCATCATCCTGTTTCTGGTGGCCACAGCCA CAGGCGTGCACAGCGATATTGTGCTGACACAGAGCCCCGCCAT CCTGAGTGCTTCTCCAGGCGAGAAAGTGACCATGACCTGCAGA GCCAGCAGCAGCGTGAACTACATGGACTGGTATCAGAAGAAG CCCGGCAGCAGCCCCAAGCCTTGGATCTACGCCACAAGCAATC TGGCCAGCGGAGTGCCTGCCAGATTTTCTGGCTCTGGCAGCGG CACAAGCTACAGCCTGACAATCAGCAGAGTGGAAGCCGAGGA TGCCGCCACCTACTACTGTCAGCAGTGGTCCTTCAATCCTCCTA CCTTCGGCGGAGGCACCAAGCTGGAAATCAAGGGCTCTACATC TGGCGGAGGTGGAAGCGGAGGCGGAGGATCTGGTGGTGGTG GATCTTCTGAGGTCCAGCTGCAACAGTCTGGCGCCGAGCTTGT GAAACCTGGCGCCTCTGTGAAGATGAGCTGCAAGGCCAGCGG CTACACCTTCACCAGCTACAACATGCACTGGGTCAAGCAGACC CCTGGACAGGGACTCGAGTGGATCGGAGCCATCTATCCCGGC AATGGCGACACCTCCTACAACCAGAAGTTCAAGGGCAAAGCCA CACTGACCGCCGACAAGAGCAGCAGCACAGCCTACATGCAGCT GAGCAGCCTGACCAGCGAGGACAGCGCCGATTACTACTGCGC CAGAAGCAACTACTACGGCAGCTCCTACTGGTTCTTCGACGTG TGGGGAGCCGGCACCACAGTGACAGTGTCCAGCAAGAAAAAG CCCCTGGACGGCGAGTACTTCACACTGCAGATCCGGGGCAGA GAACGCTTCGAGATGTTCAGAGAGCTGAACGAGGCCCTGGAA |
| | | CTGAAGGATGCCCAGGCCGGAAAAGAGCCCGGC |
| 142 | Quad 53 Oct | ATGGGCTGGTCCTGCATCATCCTGTTTCTGGTGGCCACAGCCA CAGGCGTGCACAGCGATATTGTGCTGACACAGAGCCCCGCCAT CCTGAGTGCTTCTCCAGGCGAGAAAGTGACCATGACCTGCAGA GCCAGCAGCAGCGTGAACTACATGGACTGGTATCAGAAGAAG CCCGGCAGCAGCCCCAAGCCTTGGATCTACGCCACAAGCAATC TGGCCAGCGGAGTGCCTGCCAGATTTTCTGGCTCTGGCAGCGG CACAAGCTACAGCCTGACAATCAGCAGAGTGGAAGCCGAGGA TGCCGCCACCTACTACTGTCAGCAGTGGTCCTTCAATCCTCCTA CCTTCGGCGGAGGCACCAAGCTGGAAATCAAGGGCTCTACATC TGGCGGAGGTGGAAGCGGAGGCGGAGGATCTGGTGGTGGTG GATCTTCTGAGGTCCAGCTGCAACAGTCTGGCGCCGAGCTTGT GAAACCTGGCGCCTCTGTGAAGATGAGCTGCAAGGCCAGCGG CTACACCTTCACCAGCTACAACATGCACTGGGTCAAGCAGACC CCTGGACAGGGACTCGAGTGGATCGGAGCCATCTATCCCGGC AATGGCGACACCTCCTACAACCAGAAGTTCAAGGGCAAAGCCA CACTGACCGCCGACAAGAGCAGCAGCACAGCCTACATGCAGCT GAGCAGCCTGACCAGCGAGGACAGCGCCGATTACTACTGCGC CAGAAGCAACTACTACGGCAGCTCCTACTGGTTCTTCGACGTG TGGGGAGCCGGCACCACAGTGACAGTGTCCAGCAAGAAAAAG CCCCTGGACGGCGAGTACTTCACACTGCAGATCCGGGGCAGA GAACGCTTCGAGATGTTCAGAGAGCTGAACGAGGCCCTGGAA CTGAAGGATGCCCAGGCCGGAAAAGAGCCCGGCGATATTGTG CTGACACAGAGCCCCGCCATCCTGAGTGCTTCTCCAGGCGAGA AAGTGACCATGACCTGCAGAGCCAGCAGCAGCGTGAACTACA TGGACTGGTATCAGAAGAAGCCCGGCAGCAGCCCCAAGCCTT GGATCTACGCCACAAGCAATCTGGCCAGCGGAGTGCCTGCCA GATTTTCTGGCTCTGGCAGCGGCACAAGCTACAGCCTGACAAT CAGCAGAGTGGAAGCCGAGGATGCCGCCACCTACTACTGTCA GCAGTGGTCCTTCAATCCTCCTACCTTCGGCGGAGGCACCAAG CTGGAAATCAAGGGCTCTACATCTGGCGGAGGTGGAAGCGGA |
| | | GGCGGAGGATCTGGTGGTGGTGGATCTTCTGAGGTCCAGCTG CAACAGTCTGGCGCCGAGCTTGTGAAACCTGGCGCCTCTGTGA AGATGAGCTGCAAGGCCAGCGGCTACACCTTCACCAGCTACAA CATGCACTGGGTCAAGCAGACCCCTGGACAGGGACTCGAGTG GATCGGAGCCATCTATCCCGGCAATGGCGACACCTCCTACAAC CAGAAGTTCAAGGGCAAAGCCACACTGACCGCCGACAAGAGC AGCAGCACAGCCTACATGCAGCTGAGCAGCCTGACCAGCGAG GACAGCGCCGATTACTACTGCGCCAGAAGCAACTACTACGGCA GCTCCTACTGGTTCTTCGACGTGTGGGGAGCCGGCACCACAGT GACAGTGTCCAGC |
| 143 | Quad 54 | atgggatggtcttgtataattctgttcctggtggcaacagcaacaggagtgcatagc GAGGTGCAGCTGGTTGAATCTGGCGGAGGACTGGTTCAGCCT GGCGGATCTCTGAGACTGTCTTGTGCCGCCAGCGGCTTCACCT TCAGCGACTACTGGATGTACTGGGTCCGACAGGCCCCTGGCAA AGGCCTTGAATGGGTGTCCGAGATCAACACCAACGGCCTGATC ACAAAGTACCCCGACAGCGTGAAGGGCAGATTCACCATCAGCC GGGACAACGCCAAGAACACCCTGTACCTGCAGATGAACAGCCT GCGGCCTGAGGATACCGCCGTGTACTACTGTGCCAGATCTCCC AGCGGATTCAACAGAGGCCAGGGCACACTGGTCACCGTGTCA TCTAAGAAGAAACCACTGGATGGAGAATATTTCACCCTTCAGA TCCGTGGGCGTGAGCGCTTCGAGATGTTCCGAGAGCTGAATG AGGCCTTGGAACTCAAGGATGCCCAGGCTGGGAAGGAGCCAG GGGAGGTGCAGCTGGTTGAATCTGGCGGAGGACTGGTTCAGG CTGGCGGATCTCTGAGACTGTCTTGTGCCGCCAGCGGCAGCAG CTTCAGATTCAGAGCTATGGCCTGGTACAGACAGGCCCCTGAG AAGCAGAGGGATTTCGTGGCCACCATCAACAGCCTGGGCGAG ACAACATATGCCACCGCCGTGGAAGGCCGGTTCACCATCAGCA GAGACAACGCCAAGAACACCGTGTACCTGCAGATGGACAGCC TGAAGCCTGAGGATACCGCCGTGTACTACTGCAACGAGCCCAG AGGCAATTACTGGGGCCAGGGCACACAAGTGACCGTGTCATCT CAC |
| 144 | Quad 55 | atgggatggtcttgtataattctgttcctggtggcaacagcaacaggagtgcatagc |
| | | GAGGTGCAGCTGGTGGAGTCTGGGGGAGGCTTGGTACAGCCC GGCAGGTCCCTGAGACTCTCCTGTGCGGCCTCTGGATTCACCTT TGATGATTATGCCATGCACTGGGTCCGGCAAGCTCCAGGGAAG GGCCTGGAATGGGTCTCAGCTATCACTTGGAATAGTGGTCACA TAGACTATGCGGACTCTGTGGAGGGCCGATTCACCATCTCCAG AGACAACGCCAAGAACTCCCTGTATCTGCAAATGAACAGTCTG AGAGCTGAGGATACGGCCGTATATTACTGTGCGAAAGTCTCGT ACCTTAGCACCGCGTCCTCCCTTGACTATTGGGGCCAAGGTACC CTGGTCACCGTCTCGAGTGGCGGCGGAGGCAGCGGAGGCGG AGGTTCTGGAGGAGGGGGGAGTGACATCCAGATGACCCAGTC TCCATCCTCCCTGTCTGCATCTGTAGGGGACAGAGTCACCATCA CTTGTCGGGCAAGTCAGGGCATCAGAAATTACTTAGCCTGGTA TCAGCAAAAACCAGGGAAAGCCCCTAAGCTCCTGATCTATGCT GCATCCACTTTGCAATCAGGGGTCCCATCTCGGTTCAGTGGCA GTGGATCTGGGACAGATTTCACTCTCACCATCAGCAGCCTACA GCCTGAAGATGTTGCAACTTATTACTGTCAAAGGTATAACCGT GCACCGTATACTTTTGGCCAGGGGACCAAGGTGGAAATCAAA AAGAAGAAACCACTGGATGGAGAATATTTCACCCTTCAGATCC GTGGGCGTGAGCGCTTCGAGATGTTCCGAGAGCTGAATGAGG CCTTGGAACTCAAGGATGCCCAGGCTGGGAAGGAGCCAGGGC AGGTTCAGCTGGTTCAGTCTGGCGCCGAAGTGAAGAAACCTG GCAGCAGCGTGAAGGTGTCCTGCAAGGCCAGCGGCTACAGCT TCACCGACTACCACATCCACTGGGTCCGACAGGCTCCAGGACA AGGCTTGGAATGGATGGGCGTGATCAACCCTATGTACGGCACC ACCGATTACAACCAGCGGTTCAAGGGCAGAGTGACCATCACCG CCGATGAGAGCACAAGCACCGCCTACATGGAACTGAGCAGCC TGAGAAGCGAGGACACCGCCGTGTACTACTGCGCCAGATACG ACTACTTTACCGGCACCGGGGTGTACTGGGGACAGGGAACAC TGGTCACAGTGTCCTCTGGCGGCGGAGGCAGCGGAGGCGGAG GTTCTGGAGGAGGGGGGAGTGACATCGTGATGACCCAGACAC CTCTGAGCCTGAGCGTGACACCTGGACAGCCTGCCAGCATCAG CTGCAGATCCAGCAGATCTCTGGTGCACAGCCGGGGCAATACC TACCTGCACTGGTATCTGCAGAAGCCCGGCCAGTCTCCTCAGCT |
| | | GCTGATCTACAAGGTGTCCAACCGGTTCATCGGCGTGCCCGAT AGATTTTCTGGCAGCGGCTCTGGCACCGACTTCACCCTGAAGA TCTCCAGAGTGGAAGCCGAGGACGTGGGCGTGTACTACTGTA GCCAGAGCACCCATCTGCCTTTCACCTTTGGCCAGGGCACCAA GCTGGAAATCAAGCAC |
| 145 | Quad 56 | atgggatggtcttgtataattctgttcctggtggcaacagcaacaggagtgcatagc GAGGTGCAGCTGGTGGAGTCTGGGGGAGGCTTGGTACAGCCC GGCAGGTCCCTGAGACTCTCCTGTGCGGCCTCTGGATTCACCTT TGATGATTATGCCATGCACTGGGTCCGGCAAGCTCCAGGGAAG GGCCTGGAATGGGTCTCAGCTATCACTTGGAATAGTGGTCACA TAGACTATGCGGACTCTGTGGAGGGCCGATTCACCATCTCCAG AGACAACGCCAAGAACTCCCTGTATCTGCAAATGAACAGTCTG AGAGCTGAGGATACGGCCGTATATTACTGTGCGAAAGTCTCGT ACCTTAGCACCGCGTCCTCCCTTGACTATTGGGGCCAAGGTACC CTGGTCACCGTCTCGAGTGGCGGCGGAGGCAGCGGAGGCGG AGGTTCTGGAGGAGGGGGGAGTGACATCCAGATGACCCAGTC TCCATCCTCCCTGTCTGCATCTGTAGGGGACAGAGTCACCATCA CTTGTCGGGCAAGTCAGGGCATCAGAAATTACTTAGCCTGGTA TCAGCAAAAACCAGGGAAAGCCCCTAAGCTCCTGATCTATGCT GCATCCACTTTGCAATCAGGGGTCCCATCTCGGTTCAGTGGCA GTGGATCTGGGACAGATTTCACTCTCACCATCAGCAGCCTACA GCCTGAAGATGTTGCAACTTATTACTGTCAAAGGTATAACCGT GCACCGTATACTTTTGGCCAGGGGACCAAGGTGGAAATCAAA AAGAAGAAACCACTGGATGGAGAATATTTCACCCTTCAGATCC GTGGGCGTGAGCGCTTCGAGATGTTCCGAGAGCTGAATGAGG CCTTGGAACTCAAGGATGCCCAGGCTGGGAAGGAGCCAGGGG AGGTGCAGCTGGTTGAATCTGGCGGAGGACTGGTTCAGGCTG GCGGATCTCTGAGACTGTCTTGTGCCGCCAGCGGCAGCAGCTT CAGATTCAGAGCTATGGCCTGGTACAGACAGGCCCCTGAGAA GCAGAGGGATTTCGTGGCCACCATCAACAGCCTGGGCGAGAC AACATATGCCACCGCCGTGGAAGGCCGGTTCACCATCAGCAGA GACAACGCCAAGAACACCGTGTACCTGCAGATGGACAGCCTG AAGCCTGAGGATACCGCCGTGTACTACTGCAACGAGCCCAGA |
| | | GGCAATTACTGGGGCCAGGGCACACAAGTGACCGTGTCATCTC AC |
| 146 | Quad 57 | atgggatggtcttgtataattctgttcctggtggcaacagcaacaggagtgcatagc GAGGTGCAGCTGGTTGAATCTGGCGGAGGACTGGTTCAGGCT GGCGGATCTCTGAGACTGTCTTGTGCCGCCAGCGGCAGCAGCT TCAGATTCAGAGCTATGGCCTGGTACAGACAGGCCCCTGAGAA GCAGAGGGATTTCGTGGCCACCATCAACAGCCTGGGCGAGAC AACATATGCCACCGCCGTGGAAGGCCGGTTCACCATCAGCAGA GACAACGCCAAGAACACCGTGTACCTGCAGATGGACAGCCTG AAGCCTGAGGATACCGCCGTGTACTACTGCAACGAGCCCAGA GGCAATTACTGGGGCCAGGGCACACAAGTGACCGTGTCATCTA AGAAGAAACCACTGGATGGAGAATATTTCACCCTTCAGATCCG TGGGCGTGAGCGCTTCGAGATGTTCCGAGAGCTGAATGAGGC CTTGGAACTCAAGGATGCCCAGGCTGGGAAGGAGCCAGGGCA C |
| 147 | Quad 63 | atgggatggtcttgtataattctgttcctggtggcaacagcaacaggagtgcatagc GAGGTGCAGCTGGTGGAGTCTGGGGGAGGCTTGGTACAGCCC GGCAGGTCCCTGAGACTCTCCTGTGCGGCCTCTGGATTCACCTT TGATGATTATGCCATGCACTGGGTCCGGCAAGCTCCAGGGAAG GGCCTGGAATGGGTCTCAGCTATCACTTGGAATAGTGGTCACA TAGACTATGCGGACTCTGTGGAGGGCCGATTCACCATCTCCAG AGACAACGCCAAGAACTCCCTGTATCTGCAAATGAACAGTCTG AGAGCTGAGGATACGGCCGTATATTACTGTGCGAAAGTCTCGT ACCTTAGCACCGCGTCCTCCCTTGACTATTGGGGCCAAGGTACC CTGGTCACCGTCTCGAGTGGCGGCGGAGGCAGCGGAGGCGG AGGTTCTGGAGGAGGGGGGAGTGACATCCAGATGACCCAGTC TCCATCCTCCCTGTCTGCATCTGTAGGGGACAGAGTCACCATCA CTTGTCGGGCAAGTCAGGGCATCAGAAATTACTTAGCCTGGTA TCAGCAAAAACCAGGGAAAGCCCCTAAGCTCCTGATCTATGCT GCATCCACTTTGCAATCAGGGGTCCCATCTCGGTTCAGTGGCA GTGGATCTGGGACAGATTTCACTCTCACCATCAGCAGCCTACA GCCTGAAGATGTTGCAACTTATTACTGTCAAAGGTATAACCGT |
| | | GCACCGTATACTTTTGGCCAGGGGACCAAGGTGGAAATCAAA GGTGGTGGTGGCTCCGGAGGCGGCGGCTCTGGTGGCGGTGG CAGCAAGAAGAAACCACTGGATGGAGAATATTTCACCCTTCAG ATCCGTGGGCGTGAGCGCTTCGAGATGTTCCGAGAGCTGAAT GAGGCCTTGGAACTCAAGGATGCCCAGGCTGGGAAGGAGCCA GGGCAC |
| 148 | Quad 64 | ATGGGCTGGTCCTGCATCATCCTGTTTCTGGTGGCCACAGCCA CAGGCGTGCACAGCGATATTGTGCTGACACAGAGCCCCGCCAT CCTGAGTGCTTCTCCAGGCGAGAAAGTGACCATGACCTGCAGA GCCAGCAGCAGCGTGAACTACATGGACTGGTATCAGAAGAAG CCCGGCAGCAGCCCCAAGCCTTGGATCTACGCCACAAGCAATC TGGCCAGCGGAGTGCCTGCCAGATTTTCTGGCTCTGGCAGCGG CACAAGCTACAGCCTGACAATCAGCAGAGTGGAAGCCGAGGA TGCCGCCACCTACTACTGTCAGCAGTGGTCCTTCAATCCTCCTA CCTTCGGCGGAGGCACCAAGCTGGAAATCAAGGGCTCTACATC TGGCGGAGGTGGAAGCGGAGGCGGAGGATCTGGTGGTGGTG GATCTTCTGAGGTCCAGCTGCAACAGTCTGGCGCCGAGCTTGT GAAACCTGGCGCCTCTGTGAAGATGAGCTGCAAGGCCAGCGG CTACACCTTCACCAGCTACAACATGCACTGGGTCAAGCAGACC CCTGGACAGGGACTCGAGTGGATCGGAGCCATCTATCCCGGC AATGGCGACACCTCCTACAACCAGAAGTTCAAGGGCAAAGCCA CACTGACCGCCGACAAGAGCAGCAGCACAGCCTACATGCAGCT GAGCAGCCTGACCAGCGAGGACAGCGCCGATTACTACTGCGC CAGAAGCAACTACTACGGCAGCTCCTACTGGTTCTTCGACGTG TGGGGAGCCGGCACCACAGTGACAGTGTCCAGCGGCGGAGGT GGAAGCGGAGGCGGAGGATCTGGTGGTGGTGGATCTGCCCCT GAACTGCTGGGCGGACCTTCCGTGTTCCTGTTCCCCCCAAAGCC CAAGGACACCCTGATGATCTCCCGGACCCCCGAAGTGACCTGC GTGGTGGTGGATGTGTCCCACGAGGACCCTGAAGTGAAGTTC AATTGGTACGTGGACGGCGTGGAAGTGCACAACGCCAAGACC AAGCCTAGAGAGGAACAGTACAACTCCACCTACCGGGTGGTG TCCGTGCTGACCGTGCTGCACCAGGATTGGCTGAACGGCAAAG AGTACAAGTGCAAGGTGTCCAACAAGGCCCTGCCTGCCCCCAT |
| | | CGAAAAGACCATCTCCAAGGCCAAGGGCCAGCCCCGGGAACC CCAGGTGTACACACTGCCCCCTAGCAGGGACGAGCTGACCAA GAACCAGGTGTCCCTGACCTGTCTCGTGAAAGGCTTCTACCCCT CCGATATCGCCGTGGAATGGGAGTCCAACGGCCAGCCTGAGA ACAACTACAAGACCACCCCCCCTGTGCTGGACTCCGACGGCTC ATTCTTCCTGTACAGCAAGCTGACAGTGGACAAGTCCCGGTGG CAGCAGGGCAACGTGTTCTCCTGCTCCGTGATGCACGAGGCCC TGCACAACCACTACACCCAGAAGTCCCTGTCCCTGAGCCCCGG CAAGAAGAAAAAGCCCCTGGACGGCGAGTACTTCACACTGCA GATCCGGGGCAGAGAACGCTTCGAGATGTTCAGAGAGCTGAA CGAGGCCCTGGAACTGAAGGATGCCCAGGCCGGAAAAGAGCC CGGC |
| 149 | Quad 65 | ATGGGCTGGTCCTGCATCATCCTGTTTCTGGTGGCCACAGCCA CAGGCGTGCACAGCGATATTGTGCTGACACAGAGCCCCGCCAT CCTGAGTGCTTCTCCAGGCGAGAAAGTGACCATGACCTGCAGA GCCAGCAGCAGCGTGAACTACATGGACTGGTATCAGAAGAAG CCCGGCAGCAGCCCCAAGCCTTGGATCTACGCCACAAGCAATC TGGCCAGCGGAGTGCCTGCCAGATTTTCTGGCTCTGGCAGCGG CACAAGCTACAGCCTGACAATCAGCAGAGTGGAAGCCGAGGA TGCCGCCACCTACTACTGTCAGCAGTGGTCCTTCAATCCTCCTA CCTTCGGCGGAGGCACCAAGCTGGAAATCAAGGGCTCTACATC TGGCGGAGGTGGAAGCGGAGGCGGAGGATCTGGTGGTGGTG GATCTTCTGAGGTCCAGCTGCAACAGTCTGGCGCCGAGCTTGT GAAACCTGGCGCCTCTGTGAAGATGAGCTGCAAGGCCAGCGG CTACACCTTCACCAGCTACAACATGCACTGGGTCAAGCAGACC CCTGGACAGGGACTCGAGTGGATCGGAGCCATCTATCCCGGC AATGGCGACACCTCCTACAACCAGAAGTTCAAGGGCAAAGCCA CACTGACCGCCGACAAGAGCAGCAGCACAGCCTACATGCAGCT GAGCAGCCTGACCAGCGAGGACAGCGCCGATTACTACTGCGC CAGAAGCAACTACTACGGCAGCTCCTACTGGTTCTTCGACGTG TGGGGAGCCGGCACCACAGTGACAGTGTCCAGCAAGAAAAAG CCCCTGGACGGCGAGTACTTCACACTGCAGATCCGGGGCAGA GAACGCTTCGAGATGTTCAGAGAGCTGAACGAGGCCCTGGAA |
| | | CTGAAGGATGCCCAGGCCGGAAAAGAGCCCGGCGCCCCTGAA CTGCTGGGCGGACCTTCCGTGTTCCTGTTCCCCCCAAAGCCCAA GGACACCCTGATGATCTCCCGGACCCCCGAAGTGACCTGCGTG GTGGTGGATGTGTCCCACGAGGACCCTGAAGTGAAGTTCAATT GGTACGTGGACGGCGTGGAAGTGCACAACGCCAAGACCAAGC CTAGAGAGGAACAGTACAACTCCACCTACCGGGTGGTGTCCGT GCTGACCGTGCTGCACCAGGATTGGCTGAACGGCAAAGAGTA CAAGTGCAAGGTGTCCAACAAGGCCCTGCCTGCCCCCATCGAA AAGACCATCTCCAAGGCCAAGGGCCAGCCCCGGGAACCCCAG GTGTACACACTGCCCCCTAGCAGGGACGAGCTGACCAAGAACC AGGTGTCCCTGACCTGTCTCGTGAAAGGCTTCTACCCCTCCGAT ATCGCCGTGGAATGGGAGTCCAACGGCCAGCCTGAGAACAAC TACAAGACCACCCCCCCTGTGCTGGACTCCGACGGCTCATTCTT CCTGTACAGCAAGCTGACAGTGGACAAGTCCCGGTGGCAGCA GGGCAACGTGTTCTCCTGCTCCGTGATGCACGAGGCCCTGCAC AACCACTACACCCAGAAGTCCCTGTCCCTGAGCCCCGGCAAG |
| 150 | W51ScFv | atgggatggtcttgtataattctgttcctggtggcaacagcaacaggagtgcatagc GAGGTGCAGCTGGTGGAGTCTGGGGGAGGCTTGGTACAGCCC GGCAGGTCCCTGAGACTCTCCTGTGCGGCCTCTGGATTCACCTT TGATGATTATGCCATGCACTGGGTCCGGCAAGCTCCAGGGAAG GGCCTGGAATGGGTCTCAGCTATCACTTGGAATAGTGGTCACA TAGACTATGCGGACTCTGTGGAGGGCCGATTCACCATCTCCAG AGACAACGCCAAGAACTCCCTGTATCTGCAAATGAACAGTCTG AGAGCTGAGGATACGGCCGTATATTACTGTGCGAAAGTCTCGT ACCTTAGCACCGCGTCCTCCCTTGACTATTGGGGCCAAGGTACC CTGGTCACCGTCTCGAGTGGCGGCGGAGGCAGCGGAGGCGG AGGTTCTGGAGGAGGGGGGAGTGACATCCAGATGACCCAGTC TCCATCCTCCCTGTCTGCATCTGTAGGGGACAGAGTCACCATCA CTTGTCGGGCAAGTCAGGGCATCAGAAATTACTTAGCCTGGTA TCAGCAAAAACCAGGGAAAGCCCCTAAGCTCCTGATCTATGCT GCATCCACTTTGCAATCAGGGGTCCCATCTCGGTTCAGTGGCA GTGGATCTGGGACAGATTTCACTCTCACCATCAGCAGCCTACA GCCTGAAGATGTTGCAACTTATTACTGTCAAAGGTATAACCGT |
| | | GCACCGTATACTTTTGGCCAGGGGACCAAGGTGGAAATCAAA |

TABLE 10

Amino acid sequences of mature Quad polypeptides

| | | |
|---|---|---|
| SEQ ID NO. | QUAD NO. | AMINO ACID SEQUENCE |
| 151 | Quad 51 | EVQLVESGGGLVQPGRSLRLSCAASGFTFDDYAMHWVRQA PGKGLEWVSAITWNSGHIDYADSVEGRFTISRDNAKNSLY LQMNSLRAEDTAVYYCAKVSYLSTASSLDYWGQGTLVTVS SGGGGSGGGGSGGGGSDIQMTQSPSSLSASVGDRVTITCR ASQGIRNYLAWYQQKPGKAPKLLIYAASTLQSGVPSRFSG SGSGTDFTLTISSLQPEDVATYYCQRYNRAPYTFGQGTKV EIKKKKPLDGEYFTLQIRGRERFEMFRELNEALELKDAQA GKEPG |
| 152 | Quad 53 Mon | DIVLTQSPAILSASPGEKVTMTCRASSSVNYMDWYQKKPG SSPKPWIYATSNLASGVPARFSGSGSGTSYSLTISRVEAE DAATYYCQQWSFNPPTFGGGTKLEIKGSTSGGGGSGGGGS |
| | | GGGGSSEVQLQQSGAELVKPGASVKMSCKASGYTFTSYNM HWVKQTPGQGLEWIGAIYPGNGDTSYNQKFKGKATLTADK SSSTAYMQLSSLTSEDSADYYCARSNYYGSSYWFFDVWGA |
| | | GTTVTVSS |
| 153 | Quad 53 Tet | DIVLTQSPAILSASPGEKVTMTCRASSSVNYMDWYQKKPG SSPKPWIYATSNLASGVPARFSGSGSGTSYSLTISRVEAE DAATYYCQQWSFNPPTFGGGTKLEIKGSTSGGGGSGGGGS |
| | | GGGGSSEVQLQQSGAELVKPGASVKMSCKASGYTFTSYNM HWVKQTPGQGLEWIGAIYPGNGDTSYNQKFKGKATLTADK SSSTAYMQLSSLTSEDSADYYCARSNYYGSSYWFFDVWGA |
| | | GTTVTVSSKKKPLDGEYFTLQIRGRERFEMFRELNEALEL KDAQAGKEPG |
| 154 | Quad 53 Oct | DIVLTQSPAILSASPGEKVTMTCRASSSVNYMDWYQKKPG SSPKPWIYATSNLASGVPARFSGSGSGTSYSLTISRVEAE |
| | | DAATYYCQQWSFNPPTFGGGTKLEIKGSTSGGGGSGGGGS |
| | | GGGGSSEVQLQQSGAELVKPGASVKMSCKASGYTFTSYNM HWVKQTPGQGLEWIGAIYPGNGDTSYNQKFKGKATLTADK SSSTAYMQLSSLTSEDSADYYCARSNYYGSSYWFFDVWGA |
| | | GTTVTVSSKKKPLDGEYFTLQIRGRERFEMFRELNEALEL KDAQAGKEPGDIVLTQSPAILSASPGEKVTMTCRASSSVN YMDWYQKKPGSSPKPWIYATSNLASGVPARFSGSGSGTSY |
| | | SLTISRVEAEDAATYYCQQWSFNPPTFGGGTKLEIKGSTS GGGGSGGGGSGGGGSSEVQLQQSGAELVKPGASVKMSCKA SGYTFTSYNMHWVKQTPGQGLEWIGAIYPGNGDTSYNQKF |
| | | KGKATLTADKSSSTAYMQLSSLTSEDSADYYCARSNYYGS SYWFFDVWGAGTTVTVSS |
| 155 | Quad 54 | EVQLVESGGGLVQPGGSLRLSCAASGFTFSDYWMYWVRQA PGKGLEWVSEINTNGLITKYPDSVKGRFTISRDNAKNTLY LQMNSLRPEDTAVYYCARSPSGFNRGQGTLVTVSSKKKPL |
| | | DGEYFTLQIRGRERFEMFRELNEALELKDAQAGKEPGEVQ LVESGGGLVQAGGSLRLSCAASGSSFRFRAMAWYRQAPEK QRDFVATINSLGETTYATAVEGRFTISRDNAKNTVYLQMD |
| | | SLKPEDTAVYYCNEPRGNYWGQGTQVTVSS |
| 156 | Quad 55 | EVQLVESGGGLVQPGRSLRLSCAASGFTFDDYAMHWVRQA PGKGLEWVSAITWNSGHIDYADSVEGRFTISRDNAKNSLY LQMNSLRAEDTAVYYCAKVSYLSTASSLDYWGQGTLVTVS |
| | | SGGGGSGGGGSGGGGSDIQMTQSPSSLSASVGDRVTITCR ASQGIRNYLAWYQQKPGKAPKLLIYAASTLQSGVPSRFSG SGSGTDFTLTISSLQPEDVATYYCQRYNRAPYTFGQGTKV |
| | | EIKKKKPLDGEYFTLQIRGRERFEMFRELNEALELKDAQA GKEPGQVQLVQSGAEVKKPGSSVKVSCKASGYSFTDYHIH WVRQAPGQGLEWMGVINPMYGTTDYNQRFKGRVTITADES |
| | | TSTAYMELSSLRSEDTAVYYCARYDYFTGTGVYWGQGTLV TVSSGGGGSGGGGSGGGGSDIVMTQTPLSLSVTPGQPASI |
| | | SCRSSRSLVHSRGNTYLHWYLQKPGQSPQLLIYKVSNRFI |
| | | GVPDRFSGSGSGTDFTLKISRVEAEDVGVYYCSQSTHLP |
| | | TFGQGTKLEIK |
| 157 | Quad 56 | EVQLVESGGGLVQPGRSLRLSCAASGFTFDDYAMHWVRQA PGKGLEWVSAITWNSGHIDYADSVEGRFTISRDNAKNSLY LQMNSLRAEDTAVYYCAKVSYLSTASSLDYWGQGTLVTVS |
| | | SGGGGSGGGGSGGGGSDIQMTQSPSSLSASVGDRVTITCR ASQGIRNYLAWYQQKPGKAPKLLIYAASTLQSGVPSRFSG SGSGTDFTLTISSLQPEDVATYYCQRYNRAPYTFGQGTKV |
| | | EIKKKKPLDGEYFTLQIRGRERFEMFRELNEALELKDAQA GKEPGEVQLVESGGGLVQAGGSLRLSCAASGSSFRFRAMA WYRQAPEKQRDFVATINSLGETTYATAVEGRFTISRDNAK |
| | | NTVYLQMDSLKPEDTAVYYCNEPRGNYWGQGTQVTVSS |
| 158 | Quad 57 | EVQLVESGGGLVQAGGSLRLSCAASGSSFRFRAMAWYRQA PEKQRDFVATINSLGETTYATAVEGRFTISRDNAKNTVYL QMDSLKPEDTAVYYCNEPRGNYWGQGTQVTVSSKKKPLDG |
| | | EYFTLQIRGRERFEMFRELNEALELKDAQAGKEPG |
| 159 | Quad 63 | EVQLVESGGGLVQPGRSLRLSCAASGFTFDDYAMHWVRQA PGKGLEWVSAITWNSGHIDYADSVEGRFTISRDNAKNSLY LQMNSLRAEDTAVYYCAKVSYLSTASSLDYWGQGTLVTVS |
| | | SGGGGSGGGGSGGGGSDIQMTQSPSSLSASVGDRVTITCR ASQGIRNYLAWYQQKPGKAPKLLIYAASTLQSGVPSRFSG SGSGTDFTLTISSLQPEDVATYYCQRYNRAPYTFGQGTKV |
| | | EIKGGGGSGGGGSGGGGSKKKPLDGEYFTLQIRGRERFEM FRELNEALELKDAQAGKEPG |
| 160 | Quad 64 | DIVLTQSPAILSASPGEKVTMTCRASSSVNYMDWYQKKPG SSPKPWIYATSNLASGVPARFSGSGSGTSYSLTISRVEAE DAATYYCQQWSFNPPTFGGGTKLEIKGSTSGGGGSGGGGS |
| | | GGGGSSEVQLQQSGAELVKPGASVKMSCKASGYTFTSYNM |
| | | HWVKQTPGQGLEWIGAIYPGNGDTSYNQKFKGKATLTADK SSSTAYMQLSSLTSEDSADYYCARSNYYGSSYWFFDVWGA |
| | | GTTVTVSSGGGGSGGGGSGGGGSAPELLGGPSVFLFPPKP KDTLMISRTPEVTCVVVDVSHEDPEVKFNWYVDGVEVHNA KTKPREEQYNSTYRVVSVLTVLHQDWLNGKEYKCKVSNKA |
| | | LPAPIEKTISKAKGQPREPQVYTLPPSRDELTKNQVSLTC LVKGFYPSDIAVEWESNGQPENNYKTTPPVLDSDGSFFLY SKLTVDKSRWQQGNVFSCSVMHEALHNHYTQKSLSLSPGK |
| | | KKKPLDGEYFTLQIRGRERFEMFRELNEALELKDAQAGKE PG |
| 161 | Quad 65 | DIVLTQSPAILSASPGEKVTMTCRASSSVNYMDWYQKKPG SSPKPWIYATSNLASGVPARFSGSGSGTSYSLTISRVEAE DAATYYCQQWSFNPPTFGGGTKLEIKGSTSGGGGSGGGGS |
| | | GGGGSSEVQLQQSGAELVKPGASVKMSCKASGYTFTSYNM HWVKQTPGQGLEWIGAIYPGNGDTSYNQKFKGKATLTADK SSSTAYMQLSSLTSEDSADYYCARSNYYGSSYWFFDVWGA |
| | | GTTVTVSSKKKPLDGEYFTLQIRGRERFEMFRELNEALEL KDAQAGKEPGAPELLGGPSVFLFPPKPKDTLMISRTPEVT CVVVDVSHEDPEVKFNWYVDGVEVHNAKTKPREEQYNSTY |
| | | RVVSVLTVLHQDWLNGKEYKCKVSNKALPAPIEKTISKAK GQPREPQVYTLPPSRDELTKNQVSLTCLVKGFYPSDIAVE WESNGQPENNYKTTPPVLDSDGSFFLYSKLTVDKSRWQQG |
| | | NVFSCSVMHEALHNHYTQKSLSLSPGK |
| 162 | W51ScFv | EVQLVESGGGLVQPGRSLRLSCAASGFTFDDYAMHWVRQA PGKGLEWVSAITWNSGHIDYADSVEGRFTISRDNAKNSLY LQMNSLRAEDTAVYYCAKVSYLSTASSLDYWGQGTLVTVS |
| | | SGGGGSGGGGSGGGGSDIQMTQSPSSLSASVGDRVTITCR ASQGIRNYLAWYQQKPGKAPKLLIYAASTLQSGVPSRFSG SGSGTDFTLTISSLQPEDVATYYCQRYNRAPYTFGQGTKV |
| | | EIK |

TABLE 11

ED50 of Anti-TNFa Molecules

| Anti-TNFa | ED50 (pM) |
|---|---|
| Quad 51 | 41 |
| Humira | 107 |
| W51ScFv | 246 |

TABLE 12(a)

Amino acid sequence of the hinge region of human IgG isotvpes & sequences

| | | | |
|---|---|---|---|
| Isotype | Upper Hinge | Core Hinge | Lower Hinge/CH2 Region |
| Human IgG1 | EPKSCDKTHT | CPPCP | APELLGGPSV |
| Human IgG2 | ERKCCVE | CPPCP | APPVAGPSV |
| Human IgG3 (exon 1) | ELKTPLGDTTHT | CPRCP | APELLGGPSV |
| Human IgG3 (exons 2, 3, 4) | EPKSCDTPPP | CPRCP | APELLGGPSV |
| Human IgG4 | ESKYGPP | CPSCP | APEFLGGPSV |

CXXC motif of core hinge is underlined

TABLE 12(b)

Further Hinge Sequences. Upper and Core Regions

| | |
|---|---|
| SEQ ID NO 163: | APELLGGPSV |
| SEQ ID NO 164: | PAPELLGGPSV |
| SEQ ID NO 165: | APPVAGPSV |
| SEQ ID NO 166: | PAPPVAGPSV |
| SEQ ID NO 167: | EPKSCDKTHTPAPELLGGPSV |
| SEQ ID NO 168: | EPKSCDKTHTAPELLGGPSV |
| SEQ ID NO 169: | ERKCCVEPAPPVAGPSV |
| SEQ ID NO 170: | ERKCCVEAPPVAGPSV |
| SEQ ID NO 171: | ELKTPLGDTTHTPAPELLGGPSV |
| SEQ ID NO 172: | ELKTPLGDTTHTPAPELLGGPSV |
| SEQ ID NO 173: | EPKSCDTPPPPAPELLGGPSV |
| SEQ ID NO 174: | EPKSCDTPPPAPELLGGPSV |
| SEQ ID NO 175: | APEFLGGPSV |
| SEQ ID NO 176: | PAPEFLGGPSV |
| SEQ ID NO 177: | ESKYGPPPAPEFLGGPSV |
| SEQ ID NO 178: | ESKYGPPAPEFLGGPSV |
| SEQ ID NO 179: | Humira light Chain (see Table 18) |
| SEQ ID NO 180: | CPPC |
| SEQ ID NO 181: | CPRC |
| SEQ ID NO 182: | CPSC |
| SEQ ID NO 183: | EPKSCDKTHT |
| SEQ ID NO 184: | ERKCCVE |
| SEQ ID NO 185: | ELKTPLGDTTHT |
| SEQ ID NO 186: | EPKSCDTPPP |
| SEQ ID NO 187: | ESKYGPP |

TABLE 13

UniProt accession numbers of proteins containing p53 tetramerisation domains (TD)

| | | |
|---|---|---|
| >A0A024R4C3/345-383 | >A0A2I3GNV1/391-430 | >F7FHP5/462-501 |
| >A0A059UCX8/312-350 | >A0A2I3GRR4/391-430 | >F7FN59/397-436 |
| >A0A060XH67/46-81 | >A0A2I3GX41/212-251 | >F7FN64/397-436 |
| >A0A060XN97/313-350 | >A0A2I3H7C7/391-430 | >F7FN69/212-251 |
| >A0A060Z608/94-130 | >A0A2I3HHX4/160-198 | >F7FQR2/76-114 |
| >A0A075BAE6/344-382 | >A0A2I3HR34/391-430 | >F7GA34/345-383 |
| >A0A087R7Z2/374-413 | >A0A2I3HZV6/292-331 | >F7GA47/345-383 |
| >A0A087VQ67/198-237 | >A0A2I3M190/296-334 | >F7GA51/296-334 |
| >A0A087WZU8/308-346 | >A0A2I3M2V8/212-251 | >F7GBG3/297-336 |
| >A0A087X1Q1/160-198 | >A0A2I3M6G1/345-383 | >F7GBG7/297-336 |
| >A0A087X5S1/385-424 | >A0A2I3M9J9/391-430 | >F7GBH1/374-413 |
| >A0A087XP53/284-317 | >A0A2I3MCI3/328-366 | >F7GEP9/271-310 |
| >A0A087XYP3/293-330 | >A0A2I3MEJ7/347-386 | >F7GNX0/321-359 |
| >A0A088DIC9/324-356 | >A0A2I3MFF3/298-336 | >F7GP14/311-349 |
| >A0A091CJU7/316-354 | >A0A2I3MKF2/391-430 | >F7HW67/292-335 |
| >A0A091DI49/487-525 | >A0A2I3MNM6/397-436 | >F7I720/352-391 |
| >A0A091DSC7/272-311 | >A0A2I3MNQ1/292-331 | >F7I9C6/352-391 |
| >A0A091EA06/374-413 | >A0A2I3N2E1/291-329 | >F7I9D3/297-336 |
| >A0A091G407/347-386 | >A0A2I3N5A2/345-383 | >F7I9E9/212-251 |
| >A0A091GU41/346-385 | >A0A2I3N736/391-430 | >F7IGK5/391-430 |
| >A0A091H015/371-410 | >A0A2I3N7F5/345-383 | >F8RKR1/315-351 |
| >A0A091H3D1/198-237 | >A0A2I3NGN2/242-280 | >G1K2L5/294-334 |
| >A0A091HZ84/371-410 | >A0A2I3RBD8/296-335 | >G1L1S8/391-430 |
| >A0A091IJF7/347-386 | >A0A2I3REG9/297-336 | >G1LRQ8/345-384 |
| >A0A091JY20/198-237 | >A0A2I3RJD2/345-383 | >G1MEP6/307-345 |
| >A0A091KM07/374-413 | >A0A2I3RLD5/297-336 | >G1MS14/371-410 |
| >A0A091LTD4/198-237 | >A0A2I3RMS1/296-334 | >G1MS29/207-246 |
| >A0A091LVH3/198-237 | >A0A2I3RNZ6/375-414 | >G1N6I1/293-332 |
| >A0A091M6B6/347-386 | >A0A2I3RVP4/212-251 | >G1PSQ3/371-410 |
| >A0A091MGV6/198-237 | >A0A2I3S1V8/314-352 | >G1PZ51/314-345 |
| >A0A091NGU6/286-325 | >A0A2I3SJI2/345-383 | >G1R4S5/297-336 |
| >A0A091PJY2/198-237 | >A0A2I3SPJ9/345-383 | >G1RF61/287-325 |
| >A0A091PKU4/198-237 | >A0A2I3SZA7/345-383 | >G1SEU0/317-355 |
| >A0A091QIR4/198-237 | >A0A2I3SZK4/391-430 | >G1TBP7/297-336 |
| >A0A091R450/198-237 | >A0A2I3T0Z7/391-430 | >G1U940/294-332 |
| >A0A091RK28/198-237 | >A0A2I4ANK8/293-330 | >G2HEX8/238-276 |
| >A0A091TQD5/198-237 | >A0A2I4ANL9/404-441 | >G3GT84/353-392 |
| >A0A091UUY0/347-386 | >A0A2I4ANN9/379-416 | >G3GY68/74-112 |
| >A0A091VBV4/374-413 | >A0A2I4ANQ2/291-328 | >G3IHF1/290-328 |
| >A0A091VW97/391-430 | >A0A2I4C4P9/303-338 | >G3NTK8/356-393 |
| >A0A093BPE2/104-143 | >A0A2I4C876/81-120 | >G3PK82/295-334 |
| >A0A093BXJ6/60-99 | >A0A2I4CET7/295-334 | >G3Q6V4/297-340 |
| >A0A093E9I7/198-237 | >A0A2I4CET8/386-425 | >G3Q6V7/284-327 |
| >A0A093FLV6/198-237 | >A0A2I4CEU0/291-330 | >G3QQY2/297-336 |
| >A0A093GLW0/374-413 | >A0A2I4CEU4/382-421 | >G3R2U9/319-357 |
| >A0A093HAC9/347-386 | >A0A2J8K8U5/345-383 | >G3RHQ4/345-383 |
| >A0A093HBT9/198-237 | >A0A2J8K8U6/345-383 | >G3RXL5/212-251 |
| >A0A093IHW1/104-143 | >A0A2J8K8U7/345-383 | >G3SZA7/318-357 |
| >A0A093JJ94/283-322 | >A0A2J8K8U9/296-334 | >G3T035/318-356 |
| >A0A093K3B8/391-430 | >A0A2J8K8V2/274-312 | >G3TS21/289-328 |
| >A0A093NBQ1/374-413 | >A0A2J8K8V5/296-334 | >G3TT62/376-415 |
| >A0A093PYK4/391-430 | >A0A2J8K8X6/345-383 | >G3TYZ8/391-430 |
| >A0A093R114/347-386 | >A0A2J8K8Z2/296-334 | >G3U6D1/290-329 |
| >A0A093T5L0/283-322 | >A0A2J8K8Z8/345-383 | >G3U6U6/293-332 |
| >A0A094L4V0/283-322 | >A0A2J8KPA8/308-346 | >G3UAZ0/290-329 |
| >A0A094LB61/374-413 | >A0A2J8KPB0/280-318 | >G3UDE4/288-327 |
| >A0A094MNK3/330-369 | >A0A2J8KPB3/319-357 | >G3UHE5/293-332 |
| >A0A096MBY3/303-335 | >A0A2J8KPB5/160-198 | >G3UI57/286-325 |
| >A0A096MTM8/391-430 | >A0A2J8M649/391-430 | >G3UJ00/286-325 |
| >A0A096N540/345-383 | >A0A2J8M650/391-430 | >G3UK14/298-332 |
| >A0A096P2R8/319-357 | >A0A2J8M651/293-332 | >G3ULT4/293-332 |
| >A0A099ZPQ3/374-413 | >A0A2J8M652/391-430 | >G3UYS2/296-334 |
| >A0A099ZQ65/347-386 | >A0A2J8M655/391-430 | >G3VTK8/202-241 |
| >A0A0A0AG46/374-413 | >A0A2J8M660/212-251 | >G3VZR1/347-386 |
| >A0A0A0AU13/347-386 | >A0A2J8M663/297-336 | >G3WS63/296-334 |
| >A0A0B4VEY6/209-249 | >A0A2J8M665/297-336 | >G3X6J7/346-385 |
| >A0A0B4VFS7/181-221 | >A0A2J8M666/297-336 | >G5B5D6/317-355 |
| >A0A0B7AXG3/73-113 | >A0A2J8M667/387-426 | >G5CA58/391-430 |
| >A0A0B7C164/5-45 | >A0A2J8RWD8/319-357 | >G5CBI6/345-383 |
| >A0A0C4DFW9/296-334 | >A0A2J8RWE9/308-346 | >G7MGI1/345-383 |
| >A0A0C5DEW5/307-345 | >A0A2J8RWG5/160-198 | >G7MK85/391-430 |
| >A0A0C5DID8/307-345 | >A0A2J8RWJ2/254-292 | >G7NTA7/345-383 |
| >A0A0C5E1H5/307-345 | >A0A2J8RWJ8/280-318 | >G7NYP4/391-430 |
| >A0A0D2X0F0/498-530 | >A0A2J8URM7/345-383 | >G7PTI9/319-357 |
| >A0A0D9R9K6/211-250 | >A0A2J8URN5/296-334 | >G9J1L8/307-346 |
| >A0A0D9RG12/319-357 | >A0A2J8URN6/345-383 | >G9J1L9/416-454 |
| >A0A0D9S8U4/345-383 | >A0A2J8URP0/296-334 | >G9J1M0/350-389 |
| >A0A0F6QMK2/371-410 | >A0A2J8URP1/345-383 | >G9KUQ2/301-339 |
| >A0A0F6QNH5/371-410 | >A0A2J8URP3/296-334 | >H0VHZ7/296-334 |
| >A0A0F6QPP8/371-410 | >A0A2J8URQ1/274-312 | >H0VRT2/391-430 |
| >A0A0F6QPQ1/287-326 | >A0A2J8URQ6/345-383 | >H0WNK7/294-333 |
| >A0A0F6T5N8/287-326 | >A0A2J8URQ8/345-383 | >H0WTD3/348-386 |
| >A0A0F6T5N9/287-326 | >A0A2J8WHZ2/391-430 | >H0XGB0/319-357 |
| >A0A0F7YYL7/283-323 | >A0A2J8WHZ4/391-430 | >H0YX04/347-386 |
| >A0A0F7YYL8/287-328 | >A0A2J8WHZ7/297-336 | >H0ZGH4/391-430 |
| >A0A0F7YYP8/275-315 | >A0A2J8WI03/391-430 | >H2EHT1/280-318 |
| >A0A0F7YYQ1/283-323 | >A0A2J8WI05/387-426 | >H2LHQ7/336-373 |
| >A0A0F7YYT3/270-310 | >A0A2J8WI12/297-336 | >H2LPP5/296-337 |
| >A0A0F7YYT4/350-391 | >A0A2J8WI13/391-430 | >H2LPP7/245-286 |
| >A0A0F7YYT5/288-328 | >A0A2J8WI16/212-251 | >H2MLN6/374-413 |
| >A0A0F7YYU0/270-310 | >A0A2J8WI18/297-336 | >H2MLN7/292-331 |
| >A0A0F7YYU1/287-328 | >A0A2J8WI20/297-336 | >H2MLN8/292-331 |
| >A0A0F7YYU2/288-328 | >A0A2J8WI22/293-332 | >H2MLP0/296-335 |
| >A0A0F7YYU7/275-315 | >A0A2K5DGQ7/303-331 | >H2N9D2/244-269 |
| >A0A0F7YYU8/350-391 | >A0A2K5DXG0/283-320 | >H2NSL2/319-357 |
| >A0A0F7YYU9/356-396 | >A0A2K5DXH0/149-176 | >H2PCB7/391-430 |
| >A0A0F8AIC7/289-331 | >A0A2K5E0Y4/345-383 | >H2PXV6/345-383 |
| >A0A0F8AX61/164-203 | >A0A2K5E0Y9/345-383 | >H2QC53/319-357 |
| >A0A0F8CAV8/414-453 | >A0A2K5E0Z8/296-334 | >H2QNY5/391-430 |
| >A0A0G2KB75/105-133 | >A0A2K5E105/345-383 | >H2S6K3/392-431 |
| >A0A0G2KBB2/102-133 | >A0A2K5ESD1/391-430 | >H2S6K4/298-337 |
| >A0A0H3U6U5/312-348 | >A0A2K5ESD6/217-256 | >H2S6K5/273-312 |
| >A0A0K1TP12/317-355 | >A0A2K5ESG6/297-336 | >H2S6K6/294-333 |
| >A0A0L0G1F4/366-392 | >A0A2K5ESH6/296-335 | >H2S6K7/308-347 |
| >A0A0L8G262/360-396 | >A0A2K5ESI5/391-430 | >H2U133/246-281 |
| >A0A0N7FDT7/312-348 | >A0A2K5ESI9/391-430 | >H2U 134/291-326 |
| >A0A0N8JWU8/295-331 | >A0A2K5ESJ1/391-430 | >H2U135/287-322 |
| >A0A0P6J3J0/297-336 | >A0A2K5ESJ8/377-416 | >H2UMJ4/356-393 |
| >A0A0P7UTD5/303-342 | >A0A2K5ESK8/212-251 | >H2UMJ5/351-388 |
| >A0A0P7Y6W7/139-178 | >A0A2K5F3V2/255-292 | >H2UMJ6/286-323 |
| >A0A0Q3M6G6/462-501 | >A0A2K5IAQ7/345-383 | >H2UMJ7/282-319 |
| >A0A0Q3T504/345-384 | >A0A2K5IAT1/296-334 | >H2ZGE3/352-384 |
| >A0A0R2XFU5/51-67 | >A0A2K5IAT6/299-337 | >H2ZH73/107-145 |
| >A0A0R4IHL8/385-424 | >A0A2K5IAV0/242-280 | >H2ZH74/405-443 |
| >A0A0R4IZ80/348-387 | >A0A2K5IAW1/291-329 | >H2ZH75/337-375 |
| >A0A0S2Z4N5/391-430 | >A0A2K5IB21/345-383 | >H3B1Z4/325-365 |
| >A0A0S2Z4N6/387-426 | >A0A2K5IB95/345-383 | >H3B2L6/290-329 |
| >A0A0S7IX91/41-80 | >A0A2K5JJ67/319-357 | >H3BII1/352-391 |
| >A0A0S7K6D3/282-314 | >A0A2K5JJ94/322-360 | >H3CXQ0/301-337 |
| >AOAOU1RQC9/280-318 | >A0A2K5K821/391-430 | >H3D350/357-394 |
| >A0A0U1XP71/182-219 | >A0A2K5K823/391-430 | >H3D8D5/375-414 |
| >A0A0U3KDC1/470-508 | >A0A2K5K834/297-336 | >H6U5S2/319-357 |
| >A0A0U4B546/314-351 | >A0A2K5K835/365-404 | >H6U5S3/319-357 |
| >A0A0U4D4F9/352-387 | >A0A2K5K845/296-335 | >H9FFS1/103-141 |
| >A0A141PNN3/293-332 | >A0A2K5K849/296-335 | >H9G3N4/212-237 |
| >A0A141PNN4/208-247 | >A0A2K5MIE5/212-251 | >H9G5L7/345-384 |
| >A0A142GRI7/315-351 | >A0A2K5MIF0/292-331 | >H9GVA1/99-126 |
| >A0A146NLN6/277-313 | >A0A2K5MIF3/347-386 | >H9N2D2/378-416 |
| >A0A146P3Z1/292-328 | >A0A2K5MIG1/392-431 | >H9N2D3/378-416 |
| >A0A146VRU0/254-290 | >A0A2K5MIH1/391-430 | >H9ZEQ2/293-332 |
| >A0A146VSL6/290-325 | >A0A2K5MIH4/391-430 | >I3J187/356-394 |
| >A0A146X8K2/291-330 | >A0A2K5MIH6/297-336 | >I3KRX9/307-343 |
| >A0A146X8M0/387-426 | >A0A2K5MIP6/391-430 | >I3KT80/392-431 |
| >A0A146X8S3/295-334 | >A0A2K5MWU1/345-383 | >I3LA53/311-348 |
| >A0A146X9W6/383-422 | >A0A2K5MWV4/345-383 | >I3LPD4/391-430 |
| >A0A146XA86/291-330 | >A0A2K5MWV7/345-383 | >I3LPE8/346-385 |
| >A0A146XAK7/291-330 | >A0A2K5MWW4/296-334 | >I3MLP5/391-430 |
| >A0A146XAZ2/383-422 | >A0A2K5MWX3/345-383 | >I3N5N2/317-355 |
| >A0A146XBF3/383-422 | >A0A2K5MX14/242-280 | >I3NDP5/343-381 |
| >A0A146Z8H7/322-359 | >A0A2K5MXC3/345-383 | >I7HIK9/313-350 |
| >A0A146Z8I4/293-330 | >A0A2K5MXD2/296-334 | >K1RC48/553-594 |
| >A0A146Z8Q2/224-261 | >A0A2K5N3I9/319-357 | >K7F8T8/345-384 |
| >A0A146Z9Z7/379-416 | >A0A2K5N3Z0/328-366 | >K7G3P4/312-351 |
| >A0A147ACK6/387-426 | >A0A2K5QQH4/391-430 | >K7GCN3/282-321 |
| >A0A147AT87/94-124 | >A0A2K5QQI1/382-421 | >K7PPA8/319-357 |
| >A0A147B4U1/313-350 | >A0A2K5QQI6/391-430 | >K7PPU4/319-357 |
| >A0A151MM54/441-480 | >A0A2K5QQJ5/297-336 | >K9KFA7/17-55 |
| >A0A151MMC3/437-476 | >A0A2K5QQM5/297-336 | >L5JRP0/371-410 |
| >A0A151MW63/306-341 | >A0A2K5QQM6/296-335 | >L5JZ91/308-342 |
| >A0A151N3K6/400-439 | >A0A2K5QQP2/391-430 | >L5KJ90/409-448 |
| >A0A151N3K8/361-400 | >A0A2K5QQU5/212-251 | >L5LRF0/352-391 |
| >A0A151N445/377-416 | >A0A2K5QR20/235-274 | >L5M0C7/325-357 |
| >A0A158SIS7/313-350 | >A0A2K5QYI7/345-383 | >L5MJN5/108-133 |
| >A0A167VDT2/391-430 | >A0A2K5QYL2/345-383 | >L7N1B2/317-349 |
| >A0A172Q425/316-353 | >A0A2K5QYN8/345-383 | >L7NCR5/316-355 |
| >A0A1A6HG68/222-260 | >A0A2K5QYP8/296-334 | >L7X0Y9/317-356 |
| >A0A1A7X7J4/307-348 | >A0A2K5RMZ8/321-359 | >L7X1P3/317-356 |
| >A0A1A7ZCT3/81-118 | >A0A2K5RN37/282-320 | >L7X447/317-354 |
| >A0A1A8AZS7/38-77 | >A0A2K5TIV2/415-453 | >L8IIU4/371-410 |
| >A0A1A8C8M1/255-293 | >A0A2K5VC74/297-336 | >L8IPI8/312-348 |
| >A0A1A8D5F3/314-349 | >A0A2K5VC97/292-331 | >L8IY21/346-385 |
| >A0A1A8I4T3/1-29 | >A0A2K5VCB4/212-251 | >L9JK63/422-461 |
| >A0A1A8IW64/291-330 | >A0A2K5VCD2/397-436 | >L9KM90/323-358 |
| >A0A1A8JIA0/314-349 | >A0A2K5VCE6/297-336 | >M0R497/209-247 |
| >A0A1A8JZF7/291-330 | >A0A2K5VCE8/391-430 | >M3VVE4/347-386 |
| >A0A1A8N999/312-347 | >A0A2K5VCF3/391-430 | >M3WFF4/461-500 |
| >A0A1A8P8L4/312-345 | >A0A2K5VIY5/345-383 | >M3XIT7/352-391 |
| >A0A1A8UK50/314-349 | >A0A2K5VJ29/242-280 | >M3XWF7/297-336 |
| >A0A1B1CUP8/290-331 | >A0A2K5VJ84/345-383 | >M3Y7P2/416-455 |
| >A0A1B2TT48/312-349 | >A0A2K5VJA4/345-383 | >M3YC88/301-339 |
| >A0A1B8Y052/1-27 | >A0A2K5VJA9/296-334 | >M4APR6/291-330 |
| >A0A1D5QH87/284-322 | >A0A2K5VJB1/345-383 | >M4ASF8/203-240 |
| >A0A1I7Q490/328-366 | >A0A2K5VJC2/291-329 | >M7AN48/275-314 |
| >A0A1L8FGI1/429-468 | >A0A2K5WN47/317-355 | >M7CAH2/325-364 |
| >A0A1L8FMK1/465-504 | >A0A2K5XAH3/306-345 | >O09185/319-357 |
| >A0A1L8G3W1/293-332 | >A0A2K5XAK1/306-345 | >O12946/301-329 |
| >A0A1L8G9P1/293-332 | >A0A2K5XAR2/306-345 | >O15350/345-383 |
| >A0A1L8H4Q1/128-170 | >A0A2K5XAR7/212-251 | >O36006/317-355 |
| >A0A1S2ZRH2/303-341 | >A0A2K5XAS2/306-345 | >O57538/285-316 |
| >A0A1S2ZWK7/405-444 | >A0A2K5XTB8/345-383 | >O70366/316-353 |
| >A0A1 S2ZWM0/405 -444 | >A0A2K5XTC3/296-334 | >O88898/391-430 |
| >A0A1S2ZWM2/297-336 | >A0A2K5XTC5/345-383 | >O93379/295-334 |
| >A0A1S2ZWM5/297-336 | >A0A2K5XTC7/291-329 | >P02340/313-350 |
| >A0A1 S2ZWM8/405 -444 | >A0A2K5XTF6/345-383 | >P04637/319-357 |
| >A0A1S2ZWM9/348-387 | >A0A2K5XTG5/345-383 | >P07193/290-333 |
| >A0A1S3A060/343-381 | >A0A2K6AAH4/319-357 | >P10360/300-335 |
| >A0A1S3A061/341-379 | >A0A2K6AAI9/328-366 | >P10361/317-354 |
| >A0A1S3A068/343-381 | >A0A2K6BU42/347-386 | >P13481/319-357 |
| >A0A1S3EYC1/340-378 | >A0A2K6BU55/391-430 | >P25035/320-355 |
| >A0A1S3FDV5/391-430 | >A0A2K6BU74/397-436 | >P41685/312-350 |
| >A0A1S3FDV9/391-430 | >A0A2K6BU80/212-251 | >P51664/308-345 |
| >A0A1S3FDW4/293-332 | >A0A2K6BUA9/292-331 | >P56423/319-357 |
| >A0A1S3FE88/293-332 | >A0A2K6BUB5/391-430 | >P56424/319-357 |
| >A0A1S3FE98/272-311 | >A0A2K6BUD7/391-430 | >P61260/319-357 |
| >A0A1S3FET2/297-336 | >A0A2K6BUE7/297-336 | >P67938/312-348 |
| >A0A1S3FET7/297-336 | >A0A2K6CLT3/345-383 | >P67939/312-348 |
| >A0A1S3FEU1/293-332 | >A0A2K6CLV3/345-383 | >P79734/293-333 |
| >A0A1S3FFH4/297-336 | >A0A2K6CLW2/298-336 | >P79820/291-334 |
| >A0A1S3FFH7/3 87-426 | >A0A2K6CLW4/345-383 | >P89002/304-341 |
| >A0A1S3FFI4/297-336 | >A0A2K6CLX0/296-334 | >P89003/212-249 |
| >A0A1S3FFS7/391-430 | >A0A2K6CLX4/291-329 | >P89004/212-229 |
| >A0A1S3FFT3/297-336 | >A0A2K6CLX8/345-383 | >P90332/212-249 |
| >A0A1S3GI57/345-383 | >A0A2K6CLY4/242-280 | >Q00366/322-359 |
| >A0A1S3GID5/296-334 | >A0A2K6FGK8/283-321 | >Q0GGT2/405-444 |
| >A0A1S3GJQ8/345-383 | >A0A2K6FGL2/296-334 | >Q0GMA7/311-349 |
| >A0A1S3GJR4/343-381 | >A0A2K6FGL6/283-321 | >Q0H3B6/346-384 |
| >A0A1S3II34/365-404 | >A0A2K6FGL7/283-321 | >Q0H3B7/346-384 |
| >A0A1S3IIC6/286-325 | >A0A2K6GXH2/391-430 | >Q0JRM9/357-397 |
| >A0A1S3IK29/371-410 | >A0A2K6GXH6/391-430 | >Q0Z9Z4/312-348 |
| >A0A1S3MEP3/276-315 | >A0A2K6GXJ5/391-430 | >Q1AMZ5/287-326 |
| >A0A1S3MEY9/375-414 | >A0A2K6GXK1/202-241 | >Q1AMZ6/409-448 |
| >A0A1S3MEZ2/352-391 | >A0A2K6GXL6/297-336 | >Q1AMZ7/287-326 |
| >A0A1S3MFE6/292-331 | >A0A2K6GXM1/291-330 | >Q1AMZ8/409-448 |
| >A0A1S3MQ52/298-337 | >A0A2K6GXQ4/212-251 | >Q1MSX0/308-346 |
| >A0A1S3MQ93/294-333 | >A0A2K6GXQ9/391-430 | >Q1XDZ3/350-391 |
| >A0A1S3MQE2/298-337 | >A0A2K6GZU4/311-349 | >Q27918/143-184 |
| >A0A1S3MQI7/294-333 | >A0A2K6H000/279-317 | >Q27937/344-382 |
| >A0A1S3MRC1/298-337 | >A0A2K6KG94/374-413 | >Q29475/212-250 |
| >AOA1S3N1A7/158-197 | >A0A2K6KGA5/391-430 | >Q29537/307-345 |
| >A0A1S3N1A8/164-203 | >A0A2K6KGA7/391-430 | >Q2XSC7/319-357 |
| >A0A1S3N1S0/164-203 | >A0A2K6KGB7/390-429 | >Q3UGQ1/316-353 |
| >A0A1S3N6N6/317-355 | >A0A2K6KGB9/297-336 | >Q3UVI3/293-332 |
| >A0A1S3N6V0/295-333 | >A0A2K6KGC8/297-336 | >Q4H2Z8/372-406 |
| >A0A1S3RGF1/354-389 | >A0A2K6KGD2/391-430 | >Q4H2Z9/104-147 |
| >A0A1S3RHN6/275-311 | >A0A2K6KGD4/212-251 | >Q4H300/332-375 |
| >A0A1S3SD25/319-356 | >A0A2K6KGE6/292-331 | >Q4H301/406-449 |
| >A0A1S3WGN9/343-381 | >A0A2K6KGG2/272-311 | >Q4S122/283-322 |
| >A0A1S6QMR1/317-356 | >A0A2K6L161/345-383 | >Q4S837/349-386 |
| >A0A1U7Q2G4/322-359 | >A0A2K6L165/345-383 | >Q4VR33/80-119 |
| >A0A1U7QGA5/3 87-426 | >A0A2K6L172/296-334 | >Q502Q9/294-334 |
| >A0A1U7QP43/391-430 | >A0A2K6L176/242-280 | >Q533U3/152-189 |
| >A0A1U7QRT0/391-430 | >A0A2K6L183/345-383 | >Q535D5/75-114 |
| >A0A1U7QUI2/168-206 | >A0A2K6L184/345-383 | >Q535D6/75-114 |
| >A0A1U7RA51/361-400 | >A0A2K6L191/291-329 | >Q539B9/350-389 |
| >A0A1U7RF06/348-387 | >A0A2K6LYM5/319-357 | >Q53CG6/350-389 |
| >A0A1U7RM18/352-391 | >A0A2K6NHJ2/370-409 | >Q53GA5/84-122 |
| >A0A1U7SJ11/303-338 | >A0A2K6NHJ7/297-336 | >Q549C9/316-353 |
| >A0A1U7U5H4/314-352 | >A0A2K6NHJ9/357-396 | >Q569E5/297-336 |
| >A0A1U7UA19/343-381 | >A0A2K6NHK0/212-251 | >Q5CZX0/293-332 |
| >A0A1U7UBS6/391-430 | >A0A2K6NHK3/292-331 | >Q5KQU6/352-391 |
| >A0A1U7UDX9/345-383 | >A0A2K6NHK6/370-409 | >Q5U0E4/319-357 |
| >A0A1U7UHU2/391-430 | >A0A2K6NHK7/370-409 | >Q5XHJ3/290-332 |
| >A0A1U7UHU7/297-336 | >A0A2K6NHL2/370-409 | >Q68VB0/317-355 |
| >A0A1U7ULZ4/391-430 | >A0A2K6QDI6/319-357 | >Q6DG24/292-331 |
| >A0A1U7UM51/345-383 | >A0A2K6RQ20/345-383 | >Q6NTF1/290-332 |
| >A0A1U7UNN1/345-383 | >A0A2K6RQ27/345-383 | >Q6PX73/87-126 |
| >A0A1U7UWH9/297-336 | >A0A2K6RQ35/345-383 | >Q6PX74/81-120 |
| >A0A1U8BI05/322-359 | >A0A2K6RQ45/299-337 | >Q6TDG9/40-76 |
| >A0A1U8C2W8/268-307 | >A0A2K6RQ46/296-334 | >Q6UNX2/352-391 |
| >A0A1U8DCG3/297-336 | >A0A2K6RQ50/242-280 | >Q6WG19/347-388 |
| >A0A1U9W5F4/312-349 | >A0A2K6RQ51/345-383 | >Q6WG20/347-388 |
| >A0A1V4KMC3/3 87-426 | >A0A2K6SLB6/160-198 | >Q71TU9/290-333 |
| >A0A1V4KMH⅔ 87-426 | >A0A2K6SLC5/282-320 | >Q7JP12/135-176 |
| >A0A1V4KNI0/361-400 | >A0A2K6ST65/345-383 | >Q7JP13/344-382 |
| >A0A1V9Y190/398-426 | >A0A2K6ST84/296-334 | >Q7T1D0/290-332 |
| >A0A1W4Y536/311-346 | >A0A2K6ST97/345-383 | >Q801Z7/352-391 |
| >A0A1W4YEN5/186-221 | >A0A2K6STA2/345-383 | >Q80ZA1/316-353 |
| >A0A1W4YF00/386-421 | >A0A2K6UR03/391-430 | >Q8HY32/191-229 |
| >A0A1W4YKL7/349-388 | >A0A2K6UR22/391-430 | >Q8JFE3/292-331 |
| >A0A1W4YLJ9/309-345 | >A0A2K6UR33/382-421 | >Q8JHZ5/296-335 |
| >A0A1W4YUN4/291-330 | >A0A2K6UR38/297-336 | >Q8JHZ6/292-331 |
| >A0A1W4YUY5/350-389 | >A0A2K6UR78/212-251 | >Q8SPZ3/313-351 |
| >A0A1W5AAA0/298-337 | >A0A2K6UR79/297-336 | >Q8T7V3/351-392 |
| >A0A1W5AAA4/294-333 | >A0A2K6UR80/391-430 | >Q91XH8/316-353 |
| >A0A1W5AH30/298-337 | >A0A2K6URF4/272-311 | >Q920Y0/316-353 |
| >A0A1W5AIR4/298-337 | >A5JSV4/301-336 | >Q92143/285-316 |
| >A0A1W5BGP0/491-534 | >A7S4C8/153-191 | >Q95330/317-355 |
| >A0A1W5BI93/406-449 | >A7YYJ7/292-331 | >Q98SW0/293-332 |
| >A0A1W5BJU4/491-534 | >A8DPD6/370-408 | >Q9D6A3/2-30 |
| >A0A1W5BMM1/491-534 | >A9XR54/314-345 | >Q9DEC7/293-332 |
| >A0A1W5BP67/400-443 | >B0R0M3/289-329 | >Q9EPP9/105-133 |
| >A0A1W5W828/297-335 | >B0S576/352-391 | >Q9H3D4/391-430 |
| >A0A1W6BQF7/287-324 | >B0S577/352-391 | >Q9I880/99-135 |
| >A0A1Z5LG07/266-293 | >B3GGC4/344-383 | >Q9I885/99-135 |
| >A0A210QTK4/425-465 | >B3GGC5/344-383 | >Q9JJP2/337-375 |
| >A0A212CRH3/183-222 | >B3GGC6/183-222 | >Q9JJP6/391-430 |
| >A0A212D9G7/220-245 | >B3RZS6/383-422 | >Q9N252/308-345 |
| >A0A218MZN0/291-333 | >B3TLB0/310-349 | >Q9NGC7/358-399 |
| >A0A218UKS0/168-207 | >B4DMH2/294-332 | >Q9NGC8/358-399 |
| >A0A226MN96/387-426 | >B4DNI2/309-347 | >Q9TTA1/319-357 |
| >A0A226PF87/352-391 | >B5TJK8/318-354 | >Q9TUB2/312-349 |
| >A0A250YHC8/325-363 | >B6E4X6/319-357 | >Q9TUX4/202-240 |
| >A0A286XAH3/297-336 | >B7Z8X6/297-336 | >Q9W664/353-392 |
| >A0A286XCS8/296-334 | >B8X347/358-399 | >Q9W678/289-330 |
| >A0A286XNY6/391-430 | >B8X348/358-399 | >Q9W679/304-340 |
| >A0A286XRB4/296-334 | >C0H8X1/320-355 | >Q9WUR6/317-355 |
| >A0A286XSJ9/347-385 | >C0PUM1/309-346 | >Q9XSK8/345-383 |
| >A0A286XW13/349-387 | >C3VC56/308-345 | >R0KB24/324-363 |
| >A0A286Y3E5/391-430 | >C3XPU2/393-433 | >R0KYI4/374-413 |
| >A0A287B3I9/368-405 | >C3YXH3/317-348 | >R4JHU7/351-390 |
| >A0A287BET2/370-409 | >C3ZIW1/75-106 | >R7UHV7/351-390 |
| >A0A287BN74/347-386 | >C6ERD9/314-349 | >R9TM96/107-146 |
| >A0A287D2W7/297-336 | >C9D7C9/297-336 | >R9XXS5/314-350 |
| >A0A287D527/387-426 | >C9D7D0/297-336 | >S4RR03/224-261 |
| >A0A287D7P9/391-430 | >D1LXA7/297-335 | >S7NG87/345-377 |
| >A0A293MX11/320-357 | >D2H062/371-410 | >S7NJH9/356-395 |
| >A0A2B4RUK1/425-463 | >D2HC09/337-376 | >S9WLV0/1-26 |
| >A0A2C9K4I3/338-378 | >D2HPX0/307-345 | >S9WR65/202-241 |
| >A0A2C9K4K6/482-522 | >D4AA88/390-428 | >S9YSN3/345-372 |
| >A0A2D0QVX7/291-330 | >D5KTJ0/307-344 | >T0MFN 1/322-360 |
| >A0A2D0QXV1/297-336 | >D7PQW0/201-241 | >T1EZJ4/247-278 |
| >A0A2D0QYJ0/372-411 | >E3U906/319-357 | >U3CI16/296-334 |
| >A0A2D0RBJ9/317-353 | >E5RMA8/319-357 | >U3D146/321-359 |
| >A0A2D0RXW5/294-333 | >E7FIY6/307-345 | >U3D342/293-332 |
| >A0A2D0RYT4/290-329 | >E9KN92/34-55 | >U3DZ58/345-383 |
| >A0A2D0RZF6/294-333 | >E9KN93/34-55 | >U3EAL5/387-426 |
| >A0A2D4NGD5/97-130 | >E9KN94/34-55 | >U3I844/347-386 |
| >A0A2F0B4V5/365-403 | >E9KN96/34-55 | >U3IZR9/371-410 |
| >A0A2F0BF71/1-26 | >E9KN98/34-55 | >U3JY64/391-430 |
| >A0A2F0BFK9/1-26 | >E9NME8/307-345 | >U3K4S7/359-398 |
| >A0A2G8L3N1/252-284 | >E9QG65/348-387 | >U6D8B7/305-343 |
| >A0A2G9RT86/18-57 | >F1MKA9/391-430 | >V4A869/356-396 |
| >A0A2I0LU30/391-430 | >F1NWD0/297-336 | >V8N2I9/1-18 |
| >A0A2I0LU33/293-332 | >F1P1U2/372-411 | >V8NA72/128-154 |
| >A0A2I0M380/275-314 | >F1PI27/309-347 | >V9I5U2/398-438 |
| >A0A2I0M383/297-336 | >F1PL57/391-430 | >W5KBK0/290-329 |
| >A0A2I0M3A5/297-336 | >F1PYN7/395-434 | >W5KI53/317-352 |
| >A0A2I0UNP3/391-430 | >F1QKD5/292-331 | >W5L3E6/353-392 |
| >A0A2I2U634/408-447 | >F1QLJ1/296-335 | >W5LNF4/309-348 |
| >A0A2I2UUR1/408-447 | >F1SY23/312-348 | >W5MCU3/391-430 |
| >A0A2I2Y444/391-430 | >F4YFP9/398-438 | >W5MCW2/294-333 |
| >A0A2I2Y5B0/391-430 | >F5A7P3/297-336 | >W5MD84/373-412 |
| >A0A2I2Y7Z8/280-318 | >F6MDM8/312-348 | >W5N8V4/315-352 |
| >A0A2I2YKP9/345-383 | >F6SSG7/372-406 | >W5N8V9/310-347 |
| >A0A2I2YQW0/208-247 | >F6TKT0/347-386 | >W5Q2R4/346-385 |
| >A0A2I2YW09/314-352 | >F6TL72/195-233 | >W5Q2R7/346-385 |
| >A0A2I2Z132/238-276 | >F6U7N6/202-241 | >W5QGZ8/386-425 |
| >A0A2I2Z434/391-430 | >F6VXD2/345-383 | >W5QGZ9/353-392 |
| >A0A2I2Z7P9/296-334 | >F6VXE7/242-280 | >W5U837/290-329 |
| >A0A2I2Z9N2/345-383 | >F6ZGN7/298-337 | >W5VJU6/268-305 |
| >A0A2I2ZDS0/375-414 | >F7A9M9/299-331 | >W8FSP6/309-347 |
| >A0A2I2ZIW0/288-326 | >F7A9U0/290-336 | >W8Q7P2/348-387 |
| >A0A2I2ZLA8/297-336 | >F7B2P6/352-391 | >W8Q8J6/385-424 |
| >A0A2I2ZMS7/345-383 | >F7DTE6/145-184 | >Z4YK94/344-382 |
| >A0A2I3GLU2/365-404 | >F7DUR2/389-428 | |

The amino acid sequence position of the TD domain for each protein is also indicated (ie, for the first entry, the TD sequence is amino acid residues 345 to (and including) 383 of the protein disclosed in UniProt with accession number A0A024R4C3).

### Table 14: Quad 68 & 69 Sequences

SEQ ID NO: 188 (Quad 68 nucleotide sequence)

ATGGGTTGGAGTTGTATAATTCTCTTCCTCGTCGCTACTGCTACTGGTGT
TCATTCTGAGGTCCAATTGTTGGAGTCCGGCGGCGGCGAGGTGCAACCAG
GTGGTTCACTCCGGTTGAGTTGCGCCGCGTCAGGCGGGATTTTCGCGATT
AAACCAATATCATGGTATAGGCAAGCGCCCGGGAAACAACGCGAATGGGT
GTCTACTACCACCAGTTCCGGGGCGACTAACTATGCGGAATCAGTAAAAG
GGCGCTTTACAATATCTCGCGATAATGCGAAGAATACTTTGTATTTGCAA
ATGTCATCTCTCAGGGCGGAAGACACTGCTGTTTATTATTGTAATGTCTT
TGAATACTGGGGTCAAGGTACGTTGGTGACTGTTAAGCCCGGTGGCAGTG
GCGGCTCAGAGGTTCAACTCCTTGAATCCGGAGGAGGTGAGGTCCAACCA
GGCGGAAGTCTCCGCCTTTCATGCGCAGCGTCCGGGTTTAGTTTCTCCAT
TAACGCAATGGGATGGTATCGCCAAGCACCGGGTAAAAGGCGCGAGTTCG
TTGCTGCTATTGAATCAGGTAGGAACACGGTTTACGCTGAATCCGTCAAA
GGGCGATTTACAATATCCCGTGATAATGCGAAGAATACAGTTTATTTGCA
AATGAGTTCACTCAGGGCAGAAGACACGGCGGTTTATTACTGTGGACTGC
TTAAAGGAAATCGGGTCGTCTCCCCCTCTGTCGCGTACTGGGGACAAGGA
ACCCTCGTGACCGTTAAACCCAAGAAGAAACCTCTCGATGGGGAGTACTT
CACTCTCCAAATTCGAGGTAGGGAGAGGTTTGAAATGTTTAGGGAACTCA
ATGAAGCTCTCGAGCTTAAAGACGCGCAAGCTGGTAAGGAACCAGGGCAT
CATCACCATCATCAT

SEQ ID NO: 189 (Quad 69 nucleotide sequence)

ATGGGATGGTCCTGTATTATACTCTTCTTGGTCGCAACCGCGACCGGGGT
ACATAGTGAGGTTCAACTCTTGGAGAGCGGCGGCGGCGAGGTCCAACCCG
GTGGTTCACTCAGGTTGTCCTGCGCAGCAAGTGGCGGAATTTTCGCGATT
AAACCTATTTCATGGTATAGGCAAGCTCCCGGGAAACAACGCGAATGGGT
CTCAACAACGACTTCATCTGGAGCTACAAACTATGCTGAATCAGTTAAAG
GTCGCTTTACAATTTCTCGTGATAATGCGAAGAATACTCTCTATCTGCAA
ATGTCATCATTGAGGGCTGAAGACACTGCTGTCTATTACTGTAATGTATT
TGAATACTGGGGACAAGGGACTCTCGTGACCGTTAAGCCCAAGAAGAAAC
CACTCGATGGAGAGTACTTCACTCTTCAAATACGCGGAAGGGAGCGGTTT
GAAATGTTTCGAGAATTGAATGAAGCGTTGGAGTTGAAAGACGCACAAGC
TGGCAAGGAACCAGGTGAGGTTCAATTGCTTGAGAGCGGCGGCGGGGAAG
TGCAACCAGGCGGTTCCCTCAGATTGAGCTGCGCTGCATCCGGATTTTCC
TTTTCAATTAACGCGATGGGTTGGTATCGACAAGCACCCGGGAAACGTCG
AGAGTTCGTTGCTGCGATAGAATCTGGAAGGAACACTGTTTACGCTGAAA
GTGTTAAAGGAAGGTTTACAATTTCCCGCGATAATGCAAAGAATACAGTC
TATCTTCAAATGTCCAGTCTTCGCGCGGAAGACACTGCAGTCTATTACTG
TGGTCTTCTCAAAGGGAATAGGGTTGTCTCCCCATCCGTCGCTTACTGGG
GACAAGGTACGCTCGTAACTGTCAAACCCCATCATCACCATCATCAT

SEQ ID NO: 190 (Quad 68 amino acid sequence)

MGWSCIILFLVATATGVHSEVQLLESGGGEVQPGGSLRLSCAASGGIFAI
KPISWYRQAPGKQREWVSTTTSSGATNYAESVKGRFTISRDNAKNTLYLQ
MSSLRAEDTAVYYCNVFEYWGQGTLVTVKPGGSGGSEVQLLESGGGEVQP
GGSLRLSCAASGFSFSINAMGWYRQAPGKRREFVAAIESGRNTVYAESVK
GRFTISRDNAKNTVYLQMSSLRAEDTAVYYCGLLKGNRVVSPSVAYWGQG
TLVTVKPKKKPLDGEYFTLQIRGRERFEMFRELNEALELKDAQAGKEPG

SEQ ID NO: 191 (Quad 69 amino acid sequence)

MGWSCIILFLVATATGVHSEVQLLESGGGEVQPGGSLRLSCAASGGIFAI
KPISWYRQAPGKQREWVSTTTSSGATNYAESVKGRFTISRDNAKNTLYLQ
MSSLRAEDTAVYYCNVFEYWGQGTLVTVKPKKKPLDGEYFTLQIRGRERF
EMFRELNEALELKDAQAGKEPGEVQLLESGGGEVQPGGSLRLSCAASGFS
FSINAMGWYRQAPGKRREFVAAIESGRNTVYAESVKGRFTISRDNAKNTV
YLQMSSLRAEDTAVYYCGLLKGNRVVSPSVAYWGQGTLVTVKP

TABLE 15

A table summarizing EC₅₀ values of tetravalent and octavalent Quads (non-Ig and Ig-like) anti-TNFα Quads to neutralize TNFα mediated cytotoxicity in WEHI cells.The fold enhancement in potency for the different Quads over the monovalent control is also shown

| Anti-TNFα dAb Quad | Valency | EC₅₀ (pM) | Fold enhancement of potency vs monovalent control |
|---|---|---|---|
| Anti-TNFα dAb (Monovalent Control) | 1 | 761.5 | - |
| Anti-TNFα dAb-TD | 4 | 96.6 | 8 |
| Anti-TNFα dAb-TD-dAb | 8 | 2.4 | 317 |
| Anti-TNFα dAb monomeric Ig-TD | 4 | 33.7 | 23 |
| Anti-TNFα dAb monomeric Ig-TD/dAb-Kappa | 8 | 2.8 | 272 |

TABLE 16

A table summarizing EC₅₀ values of non-Ig dodeca- and hexadeca-valent anti-TNFα Quads to neutralize TNFα mediated cytotoxicity in WEHI cells. The fold enhancement in potency for the different Quads over the monovalent control is also shown

| Anti-TNFα dAb Quad | Valency | EC₅₀ (pM) | Fold enhancement of potency vs monovalent control |
|---|---|---|---|
| Anti-TNFα dAb (Monovalent Control) | 1 | 1185 | - |
| Tandem anti-TNFα dAb-TD/ anti-TNFα-Kappa | 12 | 1.824 | 650 |
| Tandem anti-TNFα dAb-TD/ Tandem anti-TNFα-Kappa | 16 | 1.555 | 762 |
| Tandem anti-TNFα dAb-TD/ anti-TNFα- | 12 | 2.041 | 581 |
| Lambda | | | |
| Tandem anti-TNFα dAb-TD/ Tandem anti-TNFα-Lambda | 16 | 1.745 | 679 |

TABLE 17

DNA sequences encoding Quad polypeptides

| SEQ ID NO. | QUAD NO. / NAME | NUCLEOTIDE SEQUENCE |
|---|---|---|
| 192 | Q92 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTCTCGAGTGCCTCCACTAAGGGGCCGAGTGTTTTTC CACTTGCCCCATCCAGTAAGAGCACCTCTGGAGGAACTGCCGC CCTGGGTTGCCTTGTTAAGGATTACTTCCCTGAGCCAGTAACT GTTAGCTGGAACTCTGGCGCTCTGACCAGCGGAGTGCACACCT TCCCTGCTGTGCTGCAGTCCTCAGGGCTGTACTCCCTTTCTAG TGTCGTAACAGTGCCATCTTCTAGCCTGGGGACCCAGACGTAC ATCTGTAACGTGAATCATAAACCCAGTAACACAAAGGTAGATA AGAAGGTTGAACCTAAGTCCTGCGATAAGACACATACCGCCCC TGAACTGCTGGGCGGACCTTCCGTGTTCCTGTTCCCCCCAAAG CCCAAGGACACCCTGATGATCTCCCGGACCCCCGAAGTGACCT GCGTGGTGGTGGATGTGTCCCACGAGGACCCTGAAGTGAAGTT CAATTGGTACGTGGACGGCGTGGAAGTGCACAACGCCAAGACC AAGCCTAGAGAGGAACAGTACAACTCCACCTACCGGGTGGTGT CCGTGCTGACCGTGCTGCACCAGGATTGGCTGAACGGCAAAGA GTACAAGTGCAAGGTGTCCAACAAGGCCCTGCCTGCCCCCATC GAAAAGACCATCTCCAAGGCCAAGGGCCAGCCCCGGGAACCCC AGGTGTACACACTGCCCCCTAGCAGGGACGAGCTGACCAAGAA CCAGGTGTCCCTGACCTGTCTCGTGAAAGGCTTCTACCCCTCC GATATCGCCGTGGAATGGGAGTCCAACGGCCAGCCTGAGAACA |
| | | ACTACAAGACCACCCCCCCTGTGCTGGACTCCGACGGCTCATT CTTCCTGTACAGCAAGCTGACAGTGGACAAGTCCCGGTGGCAG CAGGGCAACGTGTTCTCCTGCTCCGTGATGCACGAGGCCCTGC ACAACCACTACACCCAGAAGTCCCTGTCCCTGAGCCCCGGCAA GAAGAAAAAGCCCCTGGACGGCGAGTACTTCACACTGCAGATC CGGGGCAGAGAACGCTTCGAGATGTTCAGAGAGCTGAACGAGG CCCTGGAACTGAAGGATGCCCAGGCCGGAAAAGAGCCCGGC |
| 193 | Q93 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTAAGCTCAAAACGTACGGTGGCCGCTCCCTCCGTGT TCATCTTCCCACCTTCCGACGAGCAGCTGAAGTCCGGCACCGC TTCTGTCGTGTGCCTGCTGAACAACTTCTACCCCCGCGAGGCC AAGGTGCAGTGGAAGGTGGACAACGCCCTGCAGTCCGGCAACT CCCAGGAATCCGTGACCGAGCAGGACTCCAAGGACAGCACCTA CTCCCTGTCCTCCACCCTGACCCTGTCCAAGGCCGACTACGAG AAGCACAAGGTGTACGCCTGCGAAGTGACCCACCAGGGCCTGT CTAGCCCCGTGACCAAGTCTTTCAACCGGGGCGAGTGT |
| 194 | Q113 | ATGGGTTGGTCTTGTATTATTCTTTTCCTCGTCGCAACCGCTA CCGGGGTCCATTCCGAGGTCCAATTGCTTGAATCCGGAGGAGG TGAAGTGCAACCCGGTGGGTCACTTCGGCTCTCCTGCGCCGCG AGTGGCGGGATTTTCGCTATTAAACCAATTTCCTGGTATCGTC AAGCACCAGGAAAACAACGAGAATGGGTGTCAACTACTACGTC TTCTGGGGCAACTAACTATGCAGAATCAGTCAAAGGACGCTTT ACGATTAGTCGAGATAATGCGAAGAATACTCTTTATCTCCAAA TGTCATCACTCAGGGCAGAAGACACTGCTGTCTATTATTGTAA TGTTTTCGAATACTGGGGTCAAGGAACTTTGGTGACCGTAAAG CCCGGCGGCAGCGGCGGTAGTGAAGTCCAACTCCTCGAGAGTG GAGGAGGGGAAGTGCAACCCGGCGGAAGTTTGCGGCTTAGTTG CGCAGCTTCCGGTTTTAGCTTTAGTATAAACGCAATGGGATGG |
| | | TATCGCCAAGCTCCGGGGAAAAGGCGAGAGTTCGTAGCTGCAA TTGAATCTGGACGTAACACGGTCTACGCAGAATCCGTCAAAGG GCGTTTTACTATTAGTCGCGATAATGCTAAGAATACGGTTTAT CTCCAAATGTCTTCACTCCGGGCAGAAGACACAGCTGTTTATT ACTGTGGATTGCTCAAAGGAAATCGGGTCGTCTCACCGTCAGT CGCGTACTGGGGACAAGGAACCCTTGTGACTGTTAAACCAGGT GGTGGTGGGGACAAGACCCACACCGCCCCTGAACTGCTGGGCG GACCTTCCGTGTTCCTGTTTCCTCCAAAGCCTAAGGACACCCT GATGATCAGCAGAACCCCTGAAGTGACCTGCGTGGTGGTGGAT GTGTCCCACGAGGATCCCGAAGTGAAGTTCAATTGGTACGTGG ACGGCGTGGAAGTGCACAACGCCAAGACCAAGCCTAGAGAGGA ACAGTACAACAGCACCTACAGAGTGGTGTCCGTGCTGACCGTG CTGCACCAGGATTGGCTGAACGGCAAAGAGTACAAGTGCAAGG TGTCCAACAAGGCCCTGCCTGCTCCTATCGAGAAAACCATCAG CAAGGCCAAGGGCCAGCCTAGGGAACCCCAGGTTTACACACTG CCTCCAAGCCGGGAAGAGATGACCAAGAACCAGGTGTCCCTGA CCTGCCTCGTGAAGGGCTTCTACCCTTCCGATATCGCCGTGGA ATGGGAGAGCAATGGCCAGCCAGAGAACAACTACAAGACAACC CCTCCTGTGCTGGACAGCGACGGCTCATTCTTCCTGTACAGCA AGCTGACAGTGGACAAGTCCAGATGGCAGCAGGGCAACGTGTT CTCCTGCTCTGTGATGCACGAGGCCCTGCACAACCACTACACC CAGAAGTCCCTGAGCCTGTCTCCTGGCAAAAAGAAAAAGCCCC TGGACGGCGAGTACTTCACACTGCAAATCCGGGGCAGAGAACG CTTCGAGATGTTCAGAGAGCTGAACGAGGCCCTGGAACTGAAG GATGCCCAGGCCGGAAAAGAGCCCGGC |
| 195 | Q114 | ATGGGTTGGTCTTGTATTATTCTTTTCCTCGTCGCAACCGCTA CCGGGGTCCATTCCGAGGTCCAATTGCTTGAATCCGGAGGAGG TGAAGTGCAACCCGGTGGGTCACTTCGGCTCTCCTGCGCCGCG AGTGGCGGGATTTTCGCTATTAAACCAATTTCCTGGTATCGTC AAGCACCAGGAAAACAACGAGAATGGGTGTCAACTACTACGTC TTCTGGGGCAACTAACTATGCAGAATCAGTCAAAGGACGCTTT ACGATTAGTCGAGATAATGCGAAGAATACTCTTTATCTCCAAA TGTCATCACTCAGGGCAGAAGACACTGCTGTCTATTATTGTAA TGTTTTCGAATACTGGGGTCAAGGAACTTTGGTGACCGTAAAG CCCGGCGGCAGCGGCGGTAGTGAAGTCCAACTCCTCGAGTCCG GAGGAGGTGAAGTGCAACCCGGTGGGTCACTTCGGCTCTCCTG CGCCGCGAGTGGCGGGATTTTCGCTATTAAACCAATTTCCTGG |
| | | TATCGTCAAGCACCAGGAAAACAACGAGAATGGGTGTCAACTA CTACGTCTTCTGGGGCAACTAACTATGCAGAATCAGTCAAAGG ACGCTTTACGATTAGTCGAGATAATGCGAAGAATACTCTTTAT CTCCAAATGTCATCACTCAGGGCAGAAGACACTGCTGTCTATT ATTGTAATGTTTTCGAATACTGGGGTCAAGGAACTTTGGTGAC CGTAAAGCCCGGTGGTGGTGGGGACAAGACCCACACCGCCCCT GAACTGCTGGGCGGACCTTCCGTGTTCCTGTTTCCTCCAAAGC CTAAGGACACCCTGATGATCAGCAGAACCCCTGAAGTGACCTG CGTGGTGGTGGATGTGTCCCACGAGGATCCCGAAGTGAAGTTC AATTGGTACGTGGACGGCGTGGAAGTGCACAACGCCAAGACCA AGCCTAGAGAGGAACAGTACAACAGCACCTACAGAGTGGTGTC CGTGCTGACCGTGCTGCACCAGGATTGGCTGAACGGCAAAGAG TACAAGTGCAAGGTGTCCAACAAGGCCCTGCCTGCTCCTATCG AGAAAACCATCAGCAAGGCCAAGGGCCAGCCTAGGGAACCCCA GGTTTACACACTGCCTCCAAGCCGGGAAGAGATGACCAAGAAC CAGGTGTCCCTGACCTGCCTCGTGAAGGGCTTCTACCCTTCCG ATATCGCCGTGGAATGGGAGAGCAATGGCCAGCCAGAGAACAA CTACAAGACAACCCCTCCTGTGCTGGACAGCGACGGCTCATTC TTCCTGTACAGCAAGCTGACAGTGGACAAGTCCAGATGGCAGC AGGGCAACGTGTTCTCCTGCTCTGTGATGCACGAGGCCCTGCA CAACCACTACACCCAGAAGTCCCTGAGCCTGTCTCCTGGCAAA AAGAAAAAGCCCCTGGACGGCGAGTACTTCACACTGCAAATCC GGGGCAGAGAACGCTTCGAGATGTTCAGAGAGCTGAACGAGGC CCTGGAACTGAAGGATGCCCAGGCCGGAAAAGAGCCCGGC |
| 196 | Q96 | ATGGGCTGGTCATGTATAATCCTCTTTCTTGTAGCCACAGCTA CCGGGGTACACTCAAGCGATACCGGCAGACCCTTCGTGGAAAT GTACAGCGAGATCCCCGAGATCATCCACATGACCGAGGGCAGA GAGCTGGTCATCCCCTGCAGAGTGACAAGCCCCAACATCACCG TGACTCTGAAGAAGTTCCCTCTGGACACACTGATCCCCGACGG CAAGAGAATCATCTGGGACAGCCGGAAGGGCTTCATCATCAGC AACGCCACCTACAAAGAGATCGGCCTGCTGACCTGTGAAGCCA CCGTGAATGGCCACCTGTACAAGACCAACTACCTGACACACAG ACAGACCAACACCATCATCGACGTGGTGCTGAGCCCTAGCCAC GGCATTGAACTGTCTGTGGGCGAGAAGCTGGTGCTGAACTGTA CCGCCAGAACCGAGCTGAACGTGGGCATCGACTTCAACTGGGA GTACCCCAGCAGCAAGCACCAGCACAAGAAACTGGTCAACCGG GACCTGAAAACCCAGAGCGGCAGCGAGATGAAGAAATTCCTGA |
| | | GCACCCTGACCATCGACGGCGTGACCAGATCTGACCAGGGCCT GTACACATGTGCCGCCAGCTCTGGCCTGATGACCAAGAAAAAC AGCACCTTCGTGCGGGTGCACGAGAAGGACAAGACCCACACCG CCCCTGAACTGCTGGGCGGACCTTCCGTGTTCCTGTTTCCTCC AAAGCCTAAGGACACCCTGATGATCAGCAGAACCCCTGAAGTG ACCTGCGTGGTGGTGGATGTGTCCCACGAGGATCCCGAAGTGA AGTTCAATTGGTACGTGGACGGCGTGGAAGTGCACAACGCCAA GACCAAGCCTAGAGAGGAACAGTACAATAGCACCTACAGAGTG GTGTCCGTGCTGACCGTGCTGCACCAGGATTGGCTGAACGGCA AAGAGTACAAGTGCAAGGTGTCCAACAAGGCCCTGCCTGCTCC TATCGAGAAAACCATCTCCAAGGCCAAGGGCCAGCCTAGGGAA CCCCAGGTTTACACACTGCCTCCAAGCAGGGACGAGCTGACAA AGAACCAGGTGTCCCTGACCTGCCTGGTCAAGGGCTTCTACCC TTCCGATATCGCCGTGGAATGGGAGAGCAATGGCCAGCCTGAG AACAACTACAAGACAACCCCTCCTGTGCTGGACAGCGACGGCT CATTCTTCCTGTACAGCAAGCTGACAGTGGACAAGAGCAGATG GCAGCAGGGCAACGTGTTCTCCTGCTCTGTGATGCACGAGGCC CTGCACAACCACTACACCCAGAAGTCCCTGAGCCTGTCTCCTG GAAAGAAAAAGCCCCTGGACGGCGAGTACTTCACACTGCAAAT CCGGGGCAGAGAACGCTTCGAGATGTTCAGAGAGCTGAACGAG GCCCTGGAACTGAAGGATGCCCAGGCCGGAAAAGAGCCCGGC |
| 197 | Q88 monovalent | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTAAGCTCACAC |
| 198 | Q88 tetravalent | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC |
| | | GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTAAGCTCAAAGAAGAAGCCCCTTGACGGCGAGTACT TCACACTGCAGATCCGGGGCAGAGAACGCTTCGAGATGTTCAG AGAGCTGAACGAGGCCCTGGAACTGAAGGATGCCCAGGCCGGA AAAGAGCCCGGC |
| 199 | Q88 octavalent | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTAAGCTCAAAGAAGAAGCCCCTTGACGGCGAGTACT TTACTTTGCAAATACGAGGCAGAGAAAGATTTGAAATGTTTCG GGAACTTAACGAAGCGCTGGAGCTGAAAGACGCGCAAGCCGGC AAAGAACCCGGAGAAGTTCAACTCGTCGAGAGTGGTGGCGGCT TGGTCCAACCAGGTGGGAGTCTCCGCCTTAGCTGCGCAGCAAG TGGGTTTACGTTTAGTGATTATTGGATGTATTGGGTGCGGCAA GCTCCGGGAAAGGGACTGGAGTGGGTCTCTGAAATTAACACGA ATGGTCTCATTACCAAATATCCTGATAGTGTAAAAGGACGCTT CACAATTTCTAGGGATAATGCAAAGAACACTCTCTACCTTCAA ATGAATTCCCTGCGTCCCGAAGACACGGCGGTTTATTATTGCG CTCGATCCCCTAGTGGGTTTAATCGAGGACAAGGAACCCTTGT GACAGTAAGCTCACAC |
| 200 | Q142 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC |
| | | AAATGAATTCCCTGCGTCCCGAAGACACGGCGGTTTATTATTG CGCTCGATCCCCTAGTGGGTTTAATCGAGGACAAGGAACCCTT GTGACAGTCTCGAGTGGCGGCAGCGGCGGTAGTGAAGTTCAAC TCGTCGAGAGTGGTGGCGGCTTGGTCCAACCAGGTGGGAGTCT CCGCCTTAGCTGCGCAGCAAGTGGGTTTACGTTTAGTGATTAT TGGATGTATTGGGTGCGGCAAGCTCCGGGAAAGGGACTGGAGT GGGTCTCTGAAATTAACACGAATGGTCTCATTACCAAATATCC TGATAGTGTAAAAGGACGCTTCACAATTTCTAGGGATAATGCA AAGAACACTCTCTACCTTCAAATGAATTCCCTGCGTCCCGAAG ACACGGCGGTTTATTATTGCGCTCGATCCCCTAGTGGGTTTAA TCGAGGACAAGGAACCCTTGTGACAGTCTCGTCAGGCGGAGGC GGTTCCGGAGGGGGAGGATCCGCCTCCACTAAGGGGCCGAGTG TTTTTCCACTTGCCCCATCCAGTAAGAGCACCTCTGGAGGAAC TGCCGCCCTGGGTTGCCTTGTTAAGGATTACTTCCCTGAGCCA GTAACTGTTAGCTGGAACTCTGGCGCTCTGACCAGCGGAGTGC ACACCTTCCCTGCTGTGCTGCAGTCCTCAGGGCTGTACTCCCT TTCTAGTGTCGTAACAGTGCCATCTTCTAGCCTGGGGACCCAG ACGTACATCTGTAACGTGAATCATAAACCCAGTAACACAAAGG TAGATAAGAAGGTTGAACCTAAGTCCTGCGATAAGACACATAC CAAGAAGAAACCACTGGATGGAGAATATTTCACCCTTCAGATC CGTGGGCGTGAGCGCTTCGAGATGTTCCGAGAGCTGAATGAGG CCTTGGAACTCAAGGATGCCCAGGCTGGGAAGGAGCCAGGGCA C |
| 201 | Q135 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTCTCGAGTGGCGGAGGCGGTTCCGGAGGGGGAGGAT CCGGACAGCCAAAAGCAGCCCCATCCGTAACTCTGTTCCCACC TAGTTCAGAGGAGCTTCAAGCAAACAAAGCCACACTTGTTTGC CTTATTAGTGATTTTTATCCCGGTGCCGTGACAGTTGCCTGGA AAGCTGATAGCTCACCAGTGAAAGCTGGCGTGGAGACAACCAC |
| | | ACCATCTAAACAAAGCAATAACAAGTATGCTGCCAGCTCATAT CTGAGTCTCACTCCAGAACAATGGAAGTCTCATCGGTCCTATA GCTGTCAAGTGACCCACGAAGGCAGTACCGTCGAGAAGACCGT GGCACCAACAGAGTGTAGC |
| 202 | Q136 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTCTCGAGTGGCGGAGGCGGTTCCGGAGGGGGAGGAT CCAAACGTACGGTGGCCGCTCCCTCCGTGTTCATCTTCCCACC TTCCGACGAGCAGCTGAAGTCCGGCACCGCTTCTGTCGTGTGC CTGCTGAACAACTTCTACCCCCGCGAGGCCAAGGTGCAGTGGA AGGTGGACAACGCCCTGCAATCCGGCAACTCCCAGGAATCCGT GACCGAGCAGGACTCCAAGGACAGCACCTACTCCCTGTCCTCC ACCCTGACCCTGTCCAAGGCCGACTACGAGAAGCACAAGGTGT ACGCCTGCGAAGTGACCCACCAGGGCCTGTCTAGCCCCGTGAC CAAGTCTTTCAACCGGGGCGAGTGT |
| 203 | Q144 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTCTCGAGTGGCGGCAGCGGCGGTAGTGAAGTTCAAC TCGTCGAGAGTGGTGGCGGCTTGGTCCAACCAGGTGGGAGTCT CCGCCTTAGCTGCGCAGCAAGTGGGTTTACGTTTAGTGATTAT TGGATGTATTGGGTGCGGCAAGCTCCGGGAAAGGGACTGGAGT GGGTCTCTGAAATTAACACGAATGGTCTCATTACCAAATATCC |
| | | TGATAGTGTAAAAGGACGCTTCACAATTTCTAGGGATAATGCA AAGAACACTCTCTACCTTCAAATGAATTCCCTGCGTCCCGAAG ACACGGCGGTTTATTATTGCGCTCGATCCCCTAGTGGGTTTAA TCGAGGACAAGGAACCCTTGTGACAGTCTCGTCAGGCGGAGGC GGTTCCGGAGGGGGAGGATCCGGACAGCCAAAAGCAGCCCCAT CCGTAACTCTGTTCCCACCTAGTTCAGAGGAGCTTCAAGCAAA CAAAGCCACACTTGTTTGCCTTATTAGTGATTTTTATCCCGGT GCCGTGACAGTTGCCTGGAAAGCTGATAGCTCACCAGTGAAAG CTGGCGTGGAGACAACCACACCATCTAAACAAAGCAATAACAA GTATGCTGCCAGCTCATATCTGAGTCTCACTCCAGAACAATGG AAGTCTCATCGGTCCTATAGCTGTCAAGTGACCCACGAAGGCA GTACCGTCGAGAAGACCGTGGCACCAACAGAGTGTAGC |
| 204 | Q145 | ATGGGCTGGTCATGTATAATCCTCTTTCTTGTAGCCACAGCTA CCGGGGTACACTCAGAAGTTCAACTCGTCGAGAGTGGTGGCGG CTTGGTCCAACCAGGTGGGAGTCTCCGCCTTAGCTGCGCAGCA AGTGGGTTTACGTTTAGTGATTATTGGATGTATTGGGTGCGGC AAGCTCCGGGAAAGGGACTGGAGTGGGTCTCTGAAATTAACAC GAATGGTCTCATTACCAAATATCCTGATAGTGTAAAAGGACGC TTCACAATTTCTAGGGATAATGCAAAGAACACTCTCTACCTTC AAATGAATTCCCTGCGTCCCGAAGACACGGCGGTTTATTATTG CGCTCGATCCCCTAGTGGGTTTAATCGAGGACAAGGAACCCTT GTGACAGTCTCGAGTGGCGGCAGCGGCGGTAGTGAAGTTCAAC TCGTCGAGAGTGGTGGCGGCTTGGTCCAACCAGGTGGGAGTCT CCGCCTTAGCTGCGCAGCAAGTGGGTTTACGTTTAGTGATTAT TGGATGTATTGGGTGCGGCAAGCTCCGGGAAAGGGACTGGAGT GGGTCTCTGAAATTAACACGAATGGTCTCATTACCAAATATCC TGATAGTGTAAAAGGACGCTTCACAATTTCTAGGGATAATGCA AAGAACACTCTCTACCTTCAAATGAATTCCCTGCGTCCCGAAG ACACGGCGGTTTATTATTGCGCTCGATCCCCTAGTGGGTTTAA TCGAGGACAAGGAACCCTTGTGACAGTCTCGTCAGGCGGAGGC GGTTCCGGAGGGGGAGGATCCAAACGTACGGTGGCCGCTCCCT CCGTGTTCATCTTCCCACCTTCCGACGAGCAGCTGAAGTCCGG CACCGCTTCTGTCGTGTGCCTGCTGAACAACTTCTACCCCCGC GAGGCCAAGGTGCAGTGGAAGGTGGACAACGCCCTGCAATCCG GCAACTCCCAGGAATCCGTGACCGAGCAGGACTCCAAGGACAG CACCTACTCCCTGTCCTCCACCCTGACCCTGTCCAAGGCCGAC TACGAGAAGCACAAGGTGTACGCCTGCGAAGTGACCCACCAGG |
| | | GCCTGTCTAGCCCCGTGACCAAGTCTTTCAACCGGGGCGAGTG T |
| 205 | Avelumab Fab monomeric Ig Quad (Heavy Chain) | ATGGGCTGGTCATGTATAATCCTCTTTCTTGTAGCCACAGCTA CCGGGGTACACTCAGAAGTGCAGCTGCTGGAATCTGGCGGAGG ACTGGTTCAACCTGGCGGCTCTCTGAGACTGTCTTGTGCCGCC AGCGGCTTCACCTTCAGCAGCTATATCATGATGTGGGTCCGAC AGGCCCCTGGCAAAGGCCTTGAATGGGTGTCCAGCATCTATCC CAGCGGCGGCATCACCTTTTACGCCGACACAGTGAAGGGCAGA TTCACCATCAGCCGGGACAACAGCAAGAACACCCTGTACCTGC AGATGAACAGCCTGAGAGCCGAGGACACCGCCGTGTACTACTG CGCCAGAATCAAGCTGGGCACCGTGACCACCGTGGATTATTGG GGACAGGGCACCCTGGTCACCGTCTCGAGTGCCTCCACTAAGG GGCCGAGTGTTTTTCCACTTGCCCCATCCAGTAAGAGCACCTC TGGAGGAACTGCCGCCCTGGGTTGCCTTGTTAAGGATTACTTC CCTGAGCCAGTAACTGTTAGCTGGAACTCTGGCGCTCTGACCA GCGGAGTGCACACCTTCCCTGCTGTGCTGCAGTCCTCAGGGCT GTACTCCCTTTCTAGTGTCGTAACAGTGCCATCTTCTAGCCTG GGGACCCAGACGTACATCTGTAACGTGAATCATAAACCCAGTA ACACAAAGGTAGATAAGAAGGTTGAACCTAAGTCCTGCGATAA GACACATACCGCCCCTGAACTGCTGGGCGGACCTTCCGTGTTC CTGTTCCCCCCAAAGCCCAAGGACACCCTGATGATCTCCCGGA CCCCCGAAGTGACCTGCGTGGTGGTGGATGTGTCCCACGAGGA CCCTGAAGTGAAGTTCAATTGGTACGTGGACGGCGTGGAAGTG CACAACGCCAAGACCAAGCCTAGAGAGGAACAGTACAACTCCA CCTACCGGGTGGTGTCCGTGCTGACCGTGCTGCACCAGGATTG GCTGAACGGCAAAGAGTACAAGTGCAAGGTGTCCAACAAGGCC CTGCCTGCCCCCATCGAAAAGACCATCTCCAAGGCCAAGGGCC AGCCCCGGGAACCCCAGGTGTACACACTGCCCCCTAGCAGGGA CGAGCTGACCAAGAACCAGGTGTCCCTGACCTGTCTCGTGAAA GGCTTCTACCCCTCCGATATCGCCGTGGAATGGGAGTCCAACG GCCAGCCTGAGAACAACTACAAGACCACCCCCCCTGTGCTGGA CTCCGACGGCTCATTCTTCCTGTACAGCAAGCTGACAGTGGAC AAGTCCCGGTGGCAGCAGGGCAACGTGTTCTCCTGCTCCGTGA TGCACGAGGCCCTGCACAACCACTACACCCAGAAGTCCCTGTC CCTGAGCCCCGGCAAGAAGAAAAAGCCCCTGGACGGCGAGTAC TTCACACTGCAGATCCGGGGCAGAGAACGCTTCGAGATGTTCA GAGAGCTGAACGAGGCCCTGGAACTGAAGGATGCCCAGGCCGG |
| | | AAAAGAGCCCGGC |
| 206 | Avelumab Light Chain | ATGGGCTGGTCATGTATAATCCTCTTTCTTGTAGCCACAGCTA CCGGGGTACACTCACAGTCTGCTCTGACACAGCCTGCCTCTGT GTCTGGCTCTCCTGGCCAGAGCATCACCATCAGCTGTACCGGC ACCAGCTCTGATGTCGGCGGCTACAATTACGTGTCCTGGTATC AGCAGCACCCCGGCAAGGCCCCTAAGCTGATGATCTACGACGT GTCCAACAGACCCAGCGGCGTGTCCAATAGATTCTCCGGCAGC AAGAGCGGCAACACCGCCAGCCTGACAATTAGCGGACTGCAGG CCGAGGACGAGGCCGATTACTACTGTAGCAGCTACACCAGCTC CAGCACCAGAGTGTTTGGCACCGGCACAAAAGTGACCGTGCTG GGCCAGCCTAAGGCCAATCCTACCGTGACACTGTTCCCTCCAA GCAGCGAGGAACTGCAGGCTAACAAGGCCACACTCGTGTGCCT GATCAGCGACTTTTATCCTGGCGCCGTGACCGTGGCCTGGAAG GCTGATGGATCTCCTGTGAAAGCCGGCGTGGAAACCACCAAGC CTAGCAAGCAGAGCAACAACAAATACGCCGCCAGCAGCTACCT GAGCCTGACACCTGAGCAGTGGAAGTCCCACAGATCCTACAGC TGCCAAGTGACCCACGAGGGCAGCACCGTGGAAAAAACAGTGG CCCCTACCGAGTGCAGC |
| 207 | Humira Fab monomeric Ig Quad (Heavy Chain) | ATGGGATGGTCTTGTATAATTCTGTTCCTGGTGGCAACAGCAA CAGGAGTGCATAGCGAGGTGCAGCTGGTGGAGTCTGGGGGAGG CTTGGTACAGCCCGGCAGGTCCCTGAGACTCTCCTGTGCGGCC TCTGGATTCACCTTTGATGATTATGCCATGCACTGGGTCCGGC AAGCTCCAGGGAAGGGCCTGGAATGGGTCTCAGCTATCACTTG GAATAGTGGTCACATAGACTATGCGGACTCTGTGGAGGGCCGA TTCACCATCTCCAGAGACAACGCCAAGAACTCCCTGTATCTGC AAATGAACAGTCTGAGAGCTGAGGATACGGCCGTATATTACTG TGCGAAAGTCTCGTACCTTAGCACCGCGTCCTCCCTTGACTAT TGGGGCCAAGGTACCCTGGTCACCGTCTCGAGTGCCTCCACTA AGGGGCCGAGTGTTTTTCCACTTGCCCCATCCAGTAAGAGCAC CTCTGGAGGAACTGCCGCCCTGGGTTGCCTTGTTAAGGATTAC TTCCCTGAGCCAGTAACTGTTAGCTGGAACTCTGGCGCTCTGA CCAGCGGAGTGCACACCTTCCCTGCTGTGCTGCAGTCCTCAGG GCTGTACTCCCTTTCTAGTGTCGTAACAGTGCCATCTTCTAGC CTGGGGACCCAGACGTACATCTGTAACGTGAATCATAAACCCA GTAACACAAAGGTAGATAAGAAGGTTGAACCTAAGTCCTGCGA TAAGACACATACCGCCCCTGAACTGCTGGGCGGACCTTCCGTG |
| | | TTCCTGTTCCCCCCAAAGCCCAAGGACACCCTGATGATCTCCC GGACCCCCGAAGTGACCTGCGTGGTGGTGGATGTGTCCCACGA GGACCCTGAAGTGAAGTTCAATTGGTACGTGGACGGCGTGGAA GTGCACAACGCCAAGACCAAGCCTAGAGAGGAACAGTACAACT CCACCTACCGGGTGGTGTCCGTGCTGACCGTGCTGCACCAGGA TTGGCTGAACGGCAAAGAGTACAAGTGCAAGGTGTCCAACAAG GCCCTGCCTGCCCCCATCGAAAAGACCATCTCCAAGGCCAAGG GCCAGCCCCGGGAACCCCAGGTGTACACACTGCCCCCTAGCAG GGACGAGCTGACCAAGAACCAGGTGTCCCTGACCTGTCTCGTG AAAGGCTTCTACCCCTCCGATATCGCCGTGGAATGGGAGTCCA ACGGCCAGCCTGAGAACAACTACAAGACCACCCCCCCTGTGCT GGACTCCGACGGCTCATTCTTCCTGTACAGCAAGCTGACAGTG GACAAGTCCCGGTGGCAGCAGGGCAACGTGTTCTCCTGCTCCG TGATGCACGAGGCCCTGCACAACCACTACACCCAGAAGTCCCT GTCCCTGAGCCCCGGCAAGAAGAAAAAGCCCCTGGACGGCGAG TACTTCACACTGCAGATCCGGGGCAGAGAACGCTTCGAGATGT TCAGAGAGCTGAACGAGGCCCTGGAACTGAAGGATGCCCAGGC CGGAAAAGAGCCCGGC |
| 208 | Humira Light Chain | ATGGGATGGTCTTGTATAATTCTGTTCCTGGTGGCAACAGCAA CAGGAGTGCATAGCGACATCCAGATGACCCAGTCTCCATCCTC CCTGTCTGCATCTGTAGGGGACAGAGTCACCATCACTTGTCGG GCAAGTCAGGGCATCAGAAATTACTTAGCCTGGTATCAGCAAA AACCAGGGAAAGCCCCTAAGCTCCTGATCTATGCTGCATCCAC TTTGCAATCAGGGGTCCCATCTCGGTTCAGTGGCAGTGGATCT GGGACAGATTTCACTCTCACCATCAGCAGCCTACAGCCTGAAG ATGTTGCAACTTATTACTGTCAAAGGTATAACCGTGCACCGTA TACTTTTGGCCAGGGGACCAAGGTGGAAATCAAACGTACGGTG GCCGCTCCCTCCGTGTTCATCTTCCCACCTTCCGACGAGCAGC TGAAGTCCGGCACCGCTTCTGTCGTGTGCCTGCTGAACAACTT CTACCCCCGCGAGGCCAAGGTGCAGTGGAAGGTGGACAACGCC CTGCAGTCCGGCAACTCCCAGGAATCCGTGACCGAGCAGGACT CCAAGGACAGCACCTACTCCCTGTCCTCCACCCTGACCCTGTC CAAGGCCGACTACGAGAAGCACAAGGTGTACGCCTGCGAAGTG ACCCACCAGGGCCTGTCTAGCCCCGTGACCAAGTCTTTCAACC GGGGCGAGTGT |

TABLE 18

Amino acid sequences of mature Quad polypeptides

| | | |
|---|---|---|
| SEQ ID NO. | QUAD NO. / NAME | AMINO ACID SEQUENCE |
| 209 | Q92 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSASTKGPSVFPLAPS SKSTSGGTAALGCLVKDYFPEPVTVSWNSGALTSGVHTFPAVL QSSGLYSLSSVVTVPSSSLGTQTYICNVNHKPSNTKVDKKVEP KSCDKTHTAPELLGGPSVFLFPPKPKDTLMISRTPEVTCVVVD VSHEDPEVKFNWYVDGVEVHNAKTKPREEQYNSTYRVVSVLTV LHQDWLNGKEYKCKVSNKALPAPIEKTISKAKGQPREPQVYTL PPSRDELTKNQVSLTCLVKGFYPSDIAVEWESNGQPENNYKTT PPVLDSDGSFFLYSKLTVDKSRWQQGNVFSCSVMHEALHNHYT QKSLSLSPGKKKKPLDGEYFTLQIRGRERFEMFRELNEALELK DAQAGKEPG |
| 210 | Q93 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSKRTVAAPSVFIFPP SDEQLKSGTASVVCLLNNFYPREAKVQWKVDNALQSGNSQESV TEQDSKDSTYSLSSTLTLSKADYEKHKVYACEVTHQGLSSPVT KSFNRGEC |
| 211 | Q113 | EVQLLESGGGEVQPGGSLRLSCAASGGIFAIKPISWYRQAPGK QREWVSTTTSSGATNYAESVKGRFTISRDNAKNTLYLQMSSLR AEDTAVYYCNVFEYWGQGTLVTVKPGGSGGSEVQLLESGGGEV QPGGSLRLSCAASGFSFSINAMGWYRQAPGKRREFVAAIESGR NTVYAESVKGRFTISRDNAKNTVYLQMSSLRAEDTAVYYCGLL KGNRVVSPSVAYWGQGTLVTVKPGGGGDKTHTAPELLGGPSVF LFPPKPKDTLMISRTPEVTCVVVDVSHEDPEVKFNWYVDGVEV HNAKTKPREEQYNSTYRVVSVLTVLHQDWLNGKEYKCKVSNKA LPAPIEKTISKAKGQPREPQVYTLPPSREEMTKNQVSLTCLVK |
| | | GFYPSDIAVEWESNGQPENNYKTTPPVLDSDGSFFLYSKLTVD KSRWQQGNVFSCSVMHEALHNHYTQKSLSLSPGKKKKPLDGEY FTLQIRGRERFEMFRELNEALELKDAQAGKEPG |
| 212 | Q114 | EVQLLESGGGEVQPGGSLRLSCAASGGIFAIKPISWYRQAPGK QREWVSTTTSSGATNYAESVKGRFTISRDNAKNTLYLQMSSLR AEDTAVYYCNVFEYWGQGTLVTVKPGGSGGSEVQLLESGGGEV QPGGSLRLSCAASGGIFAIKPISWYRQAPGKQREWVSTTTSSG ATNYAESVKGRFTISRDNAKNTLYLQMSSLRAEDTAVYYCNVF EYWGQGTLVTVKPGGGGDKTHTAPELLGGPSVFLFPPKPKDTL MISRTPEVTCVVVDVSHEDPEVKFNWYVDGVEVHNAKTKPREE QYNSTYRVVSVLTVLHQDWLNGKEYKCKVSNKALPAPIEKTIS KAKGQPREPQVYTLPPSREEMTKNQVSLTCLVKGFYPSDIAVE WESNGQPENNYKTTPPVLDSDGSFFLYSKLTVDKSRWQQGNVF SCSVMHEALHNHYTQKSLSLSPGKKKKPLDGEYFTLQIRGRER FEMFRELNEALELKDAQAGKEPG |
| 213 | Q96 | SDTGRPFVEMYSEIPEIIHMTEGRELVIPCRVTSPNITVTLKK FPLDTLIPDGKRIIWDSRKGFIISNATYKEIGLLTCEATVNGH LYKTNYLTHRQTNTIIDVVLSPSHGIELSVGEKLVLNCTARTE LNVGIDFNWEYPSSKHQHKKLVNRDLKTQSGSEMKKFLSTLTI DGVTRSDQGLYTCAASSGLMTKKNSTFVRVHEKDKTHTAPELL GGPSVFLFPPKPKDTLMISRTPEVTCVVVDVSHEDPEVKFNWY VDGVEVHNAKTKPREEQYNSTYRVVSVLTVLHQDWLNGKEYKC KVSNKALPAPIEKTISKAKGQPREPQVYTLPPSRDELTKNQVS LTCLVKGFYPSDIAVEWESNGQPENNYKTTPPVLDSDGSFFLY SKLTVDKSRWQQGNVFSCSVMHEALHNHYTQKSLSLSPGKKKP LDGEYFTLQIRGRERFEMFRELNEALELKDAQAGKEPG |
| 214 | Q88 monovalent | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSS |
| 215 | Q88 tetravalent | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSKKKPLDGEYFTLQI RGRERFEMFRELNEALELKDAQAGKEPG |
| 216 | Q88 octavalent | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSKKKPLDGEYFTLQI RGRERFEMFRELNEALELKDAQAGKEPGEVQLVESGGGLVQPG GSLRLSCAASGFTFSDYWMYWVRQAPGKGLEWVSEINTNGLIT KYPDSVKGRFTISRDNAKNTLYLQMNSLRPEDTAVYYCARSPS GFNRGQGTLVTVSS |
| 217 | Q142 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSGGSGGSEVQLVESG GGLVQPGGSLRLSCAASGFTFSDYWMYWVRQAPGKGLEWVSEI NTNGLITKYPDSVKGRFTISRDNAKNTLYLQMNSLRPEDTAVY YCARSPSGFNRGQGTLVTVSSGGGGSGGGGSASTKGPSVFPLA PSSKSTSGGTAALGCLVKDYFPEPVTVSWNSGALTSGVHTFPA VLQSSGLYSLSSVVTVPSSSLGTQTYICNVNHKPSNTKVDKKV EPKSCDKTHTKKKPLDGEYFTLQIRGRERFEMFRELNEALELK DAQAGKEPG |
| 218 | Q135 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSGGGGSGGGGSGQPK AAPSVTLFPPSSEELQANKATLVCLISDFYPGAVTVAWKADSS PVKAGVETTTPSKQSNNKYAASSYLSLTPEQWKSHRSYSCQVT HEGSTVEKTVAPTECS |
| 219 | Q136 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSGGGGSGGGGSKRTV AAPSVFIFPPSDEQLKSGTASVVCLLNNFYPREAKVQWKVDNA LQSGNSQESVTEQDSKDSTYSLSSTLTLSKADYEKHKVYACEV THQGLSSPVTKSFNRGEC |
| 220 | Q144 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSGGSGGSEVQLVESG GGLVQPGGSLRLSCAASGFTFSDYWMYWVRQAPGKGLEWVSEI NTNGLITKYPDSVKGRFTISRDNAKNTLYLQMNSLRPEDTAVY YCARSPSGFNRGQGTLVTVSSGGGGSGGGGSGQPKAAPSVTLF |
| | | PPSSEELQANKATLVCLISDFYPGAVTVAWKADSSPVKAGVET TTPSKQSNNKYAASSYLSLTPEQWKSHRSYSCQVTHEGSTVEK TVAPTECS |
| 221 | Q145 | EVQLVESGGGLVQPGGSLRLSCAASGFTFSDYWMYWVRQAPGK GLEWVSEINTNGLITKYPDSVKGRFTISRDNAKNTLYLQMNSL RPEDTAVYYCARSPSGFNRGQGTLVTVSSGGSGGSEVQLVESG GGLVQPGGSLRLSCAASGFTFSDYWMYWVRQAPGKGLEWVSEI NTNGLITKYPDSVKGRFTISRDNAKNTLYLQMNSLRPEDTAVY YCARSPSGFNRGQGTLVTVSSGGGGSGGGGSKRTVAAPSVFIF PPSDEQLKSGTASVVCLLNNFYPREAKVQWKVDNALQSGNSQE SVTEQDSKDSTYSLSSTLTLSKADYEKHKVYACEVTHQGLSSP VTKSFNRGEC |
| 222 | Avelumab Fab monomeric Ig Quad (Heavy Chain) | EVQLLESGGGLVQPGGSLRLSCAASGFTFSSYIMMWVRQAPGK GLEWVSSIYPSGGITFYADTVKGRFTISRDNSKNTLYLQMNSL RAEDTAVYYCARIKLGTVTTVDYWGQGTLVTVSSASTKGPSVF PLAPSSKSTSGGTAALGCLVKDYFPEPVTVSWNSGALTSGVHT FPAVLQSSGLYSLSSVVTVPSSSLGTQTYICNVNHKPSNTKVD KKVEPKSCDKTHTAPELLGGPSVFLFPPKPKDTLMISRTPEVT CVVVDVSHEDPEVKFNWYVDGVEVHNAKTKPREEQYNSTYRVV SVLTVLHQDWLNGKEYKCKVSNKALPAPIEKTISKAKGQPREP QVYTLPPSRDELTKNQVSLTCLVKGFYPSDIAVEWESNGQPEN NYKTTPPVLDSDGSFFLYSKLTVDKSRWQQGNVFSCSVMHEAL HNHYTQKSLSLSPGKKKKPLDGEYFTLQIRGRERFEMFRELNE ALELKDAQAGKEPG |
| 223 | Avelumab Light Chain | QSALTQPASVSGSPGQSITISCTGTSSDVGGYNYVSWYQQHPG KAPKLMIYDVSNRPSGVSNRFSGSKSGNTASLTISGLQAEDEA DYYCSSYTSSSTRVFGTGTKVTVLGQPKANPTVTLFPPSSEEL QANKATLVCLISDFYPGAVTVAWKADGSPVKAGVETTKPSKQS NNKYAASSYLSLTPEQWKSHRSYSCQVTHEGSTVEKTVAPTEC S |
| 224 | Humira Fab monomeric Ig Quad (Heavy | EVQLVESGGGLVQPGRSLRLSCAASGFTFDDYAMHWVRQAPGK GLEWVSAITWNSGHIDYADSVEGRFTISRDNAKNSLYLQMNSL RAEDTAVYYCAKVSYLSTASSLDYWGQGTLVTVSSASTKGPSV FPLAPSSKSTSGGTAALGCLVKDYFPEPVTVSWNSGALTSGVH TFPAVLQSSGLYSLSSVVTVPSSSLGTQTYICNVNHKPSNTKV |
| | Chain) | DKKVEPKSCDKTHTAPELLGGPSVFLFPPKPKDTLMISRTPEV TCVVVDVSHEDPEVKFNWYVDGVEVHNAKTKPREEQYNSTYRV VSVLTVLHQDWLNGKEYKCKVSNKALPAPIEKTISKAKGQPRE PQVYTLPPSRDELTKNQVSLTCLVKGFYPSDIAVEWESNGQPE NNYKTTPPVLDSDGSFFLYSKLTVDKSRWQQGNVFSCSVMHEA LHNHYTQKSLSLSPGKKKKPLDGEYFTLQIRGRERFEMFRELN EALELKDAQAGKEPG |
| 179 | Humira Light Chain | DIQMTQSPSSLSASVGDRVTITCRASQGIRNYLAWYQQKPGKA PKLLIYAASTLQSGVPSRFSGSGSGTDFTLTISSLQPEDVATY YCQRYNRAPYTFGQGTKVEIKRTVAAPSVFIFPPSDEQLKSGT ASVVCLLNNFYPREAKVQWKVDNALQSGNSQESVTEQDSKDST YSLSSTLTLSKADYEKHKVYACEVTHQGLSSPVTKSFNRGEC |
| 225 | CR3022 VH-TD Quad heavy chain (Q146A) VH is underlined; p53 TD is in bold | QMQLVQSGTEVKKPGESLKISCKGSGYGFITYWIGWVRQMPGK GLEWMGIIYPGDSETRYSPSFQGQVTISADKSINTAYLQWSSL KASDTAIYYCAGGSGISTPMDVWGQGTTVTVASTKGPSVFPLA PSSKSTSGGTAALGCLVKDYFPEPVTVSWNSGALTSGVHTFPA VLQSSGLYSLSSVVTVPSSSLGTQTYICNVNHKPSNTKVDKKV EPKSCDKTHTKKKPLDGEYFTLQIRGRERFEMFRELNEALELK DAQAGKEPG |
| 226 | >CR3022 VH monomeric Ig-TD Quad heavy chain (Q146B) VH is underlined; p53 TD is in bold | QMQLVQSGTEVKKPGESLKISCKGSGYGFITYWIGWVRQMPGK GLEWMGIIYPGDSETRYSPSFQGQVTISADKSINTAYLQWSSL KASDTAIYYCAGGSGISTPMDVWGQGTTVTVASTKGPSVFPLA PSSKSTSGGTAALGCLVKDYFPEPVTVSWNSGALTSGVHTFPA VLQSSGLYSLSSVVTVPSSSLGTQTYICNVNHKPSNTKVDKKV EPKSCDKTHTAPELLGGPSVFLFPPKPKDTLMISRTPEVTCVV VDVSHEDPEVKFNWYVDGVEVHNAKTKPREEQYNSTYRVVSVL TVLHQDWLNGKEYKCKVSNKALPAPIEKTISKAKGQPREPQVY TLPPSRDELTKNQVSLTCLVKGFYPSDIAVEWESNGQPENNYK TTPPVLDSDGSFFLYSKLTVDKSRWQQGNVFSCSVMHEALHNH YTQKSLSLSPGKKKKPLDGEYFTLQIRGRERFEMFRELNEALE LKDAQAGKEPG |
| 227 | >CR3022 VH Ig-TD Quad heavy chain (Q146C) VH is underlined; p53 TD is in bold | QMQLVQSGTEVKKPGESLKISCKGSGYGFITYWIGWVRQMPGK GLEWMGIIYPGDSETRYSPSFQGQVTISADKSINTAYLQWSSL KASDTAIYYCAGGSGISTPMDVWGQGTTVTVASTKGPSVFPLA PSSKSTSGGTAALGCLVKDYFPEPVTVSWNSGALTSGVHTFPA VLQSSGLYSLSSVVTVPSSSLGTQTYICNVNHKPSNTKVDKKV EPKSCDKTHTCPPCPAPELLGGPSVFLFPPKPKDTLMISRTPE VTCVVVDVSHEDPEVKFNWYVDGVEVHNAKTKPREEQYNSTYR VVSVLTVLHQDWLNGKEYKCKVSNKALPAPIEKTISKAKGQPR EPQVYTLPPSRDELTKNQVSLTCLVKGFYPSDIAVEWESNGQP ENNYKTTPPVLDSDGSFFLYSKLTVDKSRWQQGNVFSCSVMHE ALHNHYTQKSLSLSPGKKKKPLDGEYFTLQIRGRERFEMFREL NEALELKDAQAGKEPG |
| 228 | >CR3022 VL Quad light chain (Q147) VL is underlined | DIQLTQSPDSLAVSLGERATINCKSSQSVLYSSINKNYLAWYQ QKPGQPPKLLIYWASTRESGVPDRFSGSGSGTDFTLTISSLQA EDVAVYYCQQYYSTPYTFGQGTKVEIKRTVAAPSVFIFPPSDE QLKSGTASVVCLLNNFYPREAKVQWKVDNALQSGNSQESVTEQ DSKDSTYSLSSTLTLSKADYEKHKVYACEVTHQGLSSPVTKSF NRGEC |
| 229 | >ACE2-TD (Q154A) ACE2 peptide is underlined; p53 TD is in bold | QSTIEEQAKTFLDKFNHEAEDLFYQSSLASWNYNTNITEENVQ NMNNAGDKWSAFLKEQSTLAQMYPLQEIQNLTVKLQLQALQQN GSSVLSEDKSKRLNTILNTMSTIYSTGKVCNPDNPQECLLLEP GLNEIMANSLDYNERLWAWESWRSEVGKQLRPLYEEYVVLKNE MARANHYEDYGDYWRGDYEVNGVDGYDYSRGQLIEDVEHTFEE IKPLYEHLHAYVRAKLMNAYPSYISPIGCLPAHLLGDMWGRFW TNLYSLTVPFGQKPNIDVTDAMVDQAWDAQRIFKEAEKFFVSV GLPNMTQGFWENSMLTDPGNVQKAVCHPTAWDLGKGDFRILMC TKVTMDDFLTAHHEMGHIQYDMAYAAQPFLLRNGANEGFHEAV GEIMSLSAATPKHLKSIGLLSPDFQEDNETEINFLLKQALTIV GTLPFTYMLEKWRWMVFKGEIPKDQWMKKWWEMKREIVGVVEP VPHDETYCDPASLFHVSNDYSFIRYYTRTLYQFQFQEALCQAA KHEGPLHKCDISNSTEAGQKLFNMLRLGKSEPWTLALENVVGA |
| | | KNMNVRPLLNYFEPLFTWLKDQNKNSFVGWSTDWSPYAD KKKP LDGEYFTLQIRGRERFEMFRELNEALELKDAQAGKEPG |
| 230 | > ACE2 monomeric Ig-TD (Q154A) ACE2 peptide is underlined; p53 TD is in bold | QSTIEEQAKTFLDKFNHEAEDLFYQSSLASWNYNTNITEENVQ NMNNAGDKWSAFLKEQSTLAQMYPLQEIQNLTVKLQLQALQQN GSSVLSEDKSKRLNTILNTMSTIYSTGKVCNPDNPQECLLLEP GLNEIMANSLDYNERLWAWESWRSEVGKQLRPLYEEYVVLKNE MARANHYEDYGDYWRGDYEVNGVDGYDYSRGQLIEDVEHTFEE IKPLYEHLHAYVRAKLMNAYPSYISPIGCLPAHLLGDMWGRFW TNLYSLTVPFGQKPNIDVTDAMVDQAWDAQRIFKEAEKFFVSV GLPNMTQGFWENSMLTDPGNVQKAVCHPTAWDLGKGDFRILMC TKVTMDDFLTAHHEMGHIQYDMAYAAQPFLLRNGANEGFHEAV GEIMSLSAATPKHLKSIGLLSPDFQEDNETEINFLLKQALTIV GTLPFTYMLEKWRWMVFKGEIPKDQWMKKWWEMKREIVGVVEP VPHDETYCDPASLFHVSNDYSFIRYYTRTLYQFQFQEALCQAA KHEGPLHKCDISNSTEAGQKLFNMLRLGKSEPWTLALENVVGA KNMNVRPLLNYFEPLFTWLKDQNKNSFVGWSTDWSPYADEPKS CDKTHTAPELLGGPSVFLFPPKPKDTLMISRTPEVTCVVVDVS HEDPEVKFNWYVDGVEVHNAKTKPREEQYNSTYRVVSVLTVLH QDWLNGKEYKCKVSNKALPAPIEKTISKAKGQPREPQVYTLPP SRDELTKNQVSLTCLVKGFYPSDIAVEWESNGQPENNYKTTPP VLDSDGSFFLYSKLTVDKSRWQQGNVFSCSVMHEALHNHYTQK SLSLSPGKKKKPLDGEYFTLQIRGRERFEMFRELNEALELKDA QAGKEPG |
| 231 | ACE2 Ig-TD (Q 154C) ACE2 peptide is underlined; p53 TD is in | QSTIEEQAKTFLDKFNHEAEDLFYQSSLASWNYNTNITEENVQ NMNNAGDKWSAFLKEQSTLAQMYPLQEIQNLTVKLQLQALQQN GSSVLSEDKSKRLNTILNTMSTIYSTGKVCNPDNPQECLLLEP GLNEIMANSLDYNERLWAWESWRSEVGKQLRPLYEEYVVLKNE MARANHYEDYGDYWRGDYEVNGVDGYDYSRGQLIEDVEHTFEE IKPLYEHLHAYVRAKLMNAYPSYISPIGCLPAHLLGDMWGRFW TNLYSLTVPFGQKPNIDVTDAMVDQAWDAQRIFKEAEKFFVSV GLPNMTQGFWENSMLTDPGNVQKAVCHPTAWDLGKGDFRILMC TKVTMDDFLTAHHEMGHIQYDMAYAAQPFLLRNGANEGFHEAV GEIMSLSAATPKHLKSIGLLSPDFQEDNETEINFLLKQALTIV |
| | bold | GTLPFTYMLEKWRWMVFKGEIPKDQWMKKWWEMKREIVGVVEP VPHDETYCDPASLFHVSNDYSFIRYYTRTLYQFQFQEALCQAA KHEGPLHKCDISNSTEAGQKLFNMLRLGKSEPWTLALENVVGA KNMNVRPLLNYFEPLFTWLKDQNKNSFVGWSTDWSPYADASTK GPSVFPLAPSSKSTSGGTAALGCLVKDYFPEPVTVSWNSGALT SGVHTFPAVLQSSGLYSLSSVVTVPSSSLGTQTYICNVNHKPS NTKVDKKVEPKSCDKTHTCPPCPAPELLGGPSVFLFPPKPKDT LMISRTPEVTCVVVDVSHEDPEVKFNWYVDGVEVHNAKTKPRE EQYNSTYRVVSVLTVLHQDWLNGKEYKCKVSNKALPAPIEKTI SKAKGQPREPQVYTLPPSRDELTKNQVSLTCLVKGFYPSDIAV EWESNGQPENNYKTTPPVLDSDGSFFLYSKLTVDKSRWQQGNV FSCSVMHEALHNHYTQKSLSLSPGKKKKPLDGEYFTLQIRGRE RFEMFRELNEALELKDAQAGKEPG |

TABLE 19

List of viruses that can be bound by a multimer of the invention
Virus
Adeno-associated virus
Adenovirus
African swine fever virus
Autographa californica multiple nucleopolyhedrovirus
Barmah Forest virus (BFV)
Bombyx mori nucleopolyhedrovirus
Chickenpox
Chikungunya alphavirus
Chikungunya Virus Infection
COVID-19 (Coronavirus SARS-Cov-2)
Coronavirus SARS-Cov-1
Coxsackieviruses
Cytomegalovirus (CMV)
Dengue virus (DENV)
Ebola
Enterovirus A & B
Epstein-Barr virus infection (EBV)
Hanta virus
Hantavirus Pulmonary Syndrome
Hepatitis virus (A, B, C, D or E)
Human herpesvirus 6 (HHV-6)
Human immunodeficiency virus (HIV)
Human papillomavirus
Human T- cell leukemia virus, type 1 (HTLV-1)
Infectious spleen and kidney necrosis virus
Influenza virus
Japanese encephalitis virus (JEV)
Kaposi’s sarcoma-associated herpesvirus KSHV (HHV-8)
Mayaro virus (MAYV)
Measles
Merkel cell polyomavirus
Mumps
Norovirus
O′nyong-nyong virus (ONNV)
Orthmyxoviruse s
Paramyxoviruses
Poliovirus
Rrabies
Respiratory syncytial virus
Retroviruses
Rhabdoviruses
Ross River virus (RRV)
Rubella
Severe acute respiratory syndrome (SARS)
Shingles
Simian immunodeficiency virus (SIV)
Simian varicella virus (SVV)
Simian Virus 40
Sindbis virus (SINV)
Singapore grouper iridovirus
Smallpox
Vaccinia virus
Varicella-zoster virus (VZV)
Vesicular stomatitis virus
West Nile virus (WNV)
Zika virus (ZIKV)

TABLE 20

List of example cell surface antigens on host cells or the surface of viruses that could be specifically bound by a multimer of the invention, eg, to block attachment onto and/or entry into host cells
Targets to block virus attachment/entry into host cells
CCR5
CCR5
CD21
CD4
CD46
CD80
CD86
Coxsackie-adenovirus receptor
CR2
CXCR4
DC-SIGN
DC-SIGNR
EGFR
G protein to cell surface receptors
GD1a glycan
Glycoproteins gB, gC, gD, gH and gL
Glycosaminoglycan
gp120
Hemagglutinin (HA1 and HA2)
Heparan sulphate
Heparan sulphate proteoglycans
Human mannose receptor C-type 1
Integrin α3β1
Integrins
Mannose-6-phosphate receptor
MHC1-α2
Myelin-associated glycoprotein
N-acetilneuraminic acid receptor
Nucleolin
Paired immunoglobulin-like receptor alpha
Phosphatidylserine
Proteoglycans
RIG-I
Sialic acid sugars and glycolipids
Surface heparan sulphate
TLR-2
TLR-3
TLR-7
TLR9
VCAM-1
xCT
αvβ3- and αvβ5-integrins
β1 and β3 integrins

### Table 21: Example Sources of Binding Sites for Multimers

(a) Taken from Trends Immunol. 2020 Apr 24, doi: 10.1016/j.it.2020.04.008, “Neutralizing Antibodies against SARS-CoV-2 and Other Human Coronaviruses”, Trends in Immunology 41, 355-359; 2020, Shibo Jiang, Christopher Hillyer, and Lanying Du, which is incorporated herein in its entirety by reference (including the references cited therein (to which the numbers in the last column of this table refers) and antibody and V domain sequences therein).

| Ab name | Source | Neutralizing activity | Neutralizing | Protective efficacy | Refs^{b} |
|---|---|---|---|---|---|
| S230.15 m396 mAbs | Human | Neutralize human (strains GD03, Urbani, Tor2) and palm civet (strains SZ3, 5Z16) SARS-CoV infection | Recognize epitopes (residues 408, 442, 443, 460, 475) on SARS-CoV S1 protein, interfering with RBD-ACE2 | Protect mice against challenge of SARS-CoV (strains Urbani, rGD03, or rSZ16) | [9] |
| S109.8 S227.14 S230.15 mAbs | Human | Neutralize human (Urbani, GZ02, CUHK-W1), palm civet (HC/SZ/61/03), and raccoon dog (A031G) SARS-CoV infectious clones containing S variants | Inhibit the binding of SARS-CoV RBD-ACE2 receptor | Protect mice against challenge of SARS-CoV infectious clones (Urbani, GZ02, HC/SZ/61/03) or mouse-adapted strain (MA15) | [10] |
| 80R scFv, mAb | Human | Neutralize live SARS-CoV (strain Urbani) infection | Recognize epitopes on SARS-CoV S1 (residues 261-672), blocking RBD-ACE2 binding and inhibiting syncytium formation | NA | [11] |
| CR3022 CR3014 scFv, mAb | Human | Neutralize live SARS-CoV (strain HKU-39849) infection; CR3022 could neutralize CR3014 escape variants | Recognize epitopes on SARS-CoV RBD (residues 318-510); CR3022 binds SARS-CoV-2 RBD with high affinity | CR3014 protects ferrets against SARS-CoV (strain HKU-39849) infection | [13] |
| 33G4 35B5 30F9 mAbs | Mouse | Neutralize human (strains GD03, Tor2) and palm civet (SZ3) pseudotyped SARS-CoV infection | Recognize epitopes on SARS-CoV RBD, blocking RBD-ACE2 receptor binding | NA | [12] |
| MERS-27 m336 MERS-GD27 MCA1 mAbs, Fabs | Human | Neutralize divergent strains ofpseudotyped and live (strain EMC2012) MERS-CoV infection | Recognize a number of key epitopes on MERS-CoV RBD protein, blocking RBD-DPP4 receptor binding | Prophylactically and therapeutically prevent and treat MERS-CoV (strain EMC2012) challenge in hDPP4-Tg mice, rabbits, or common marmosets | [3,8] |

### Table 21 Continued

(b) DNA sequences encoding the VH and VL of VH/VL binding sites for Anti-SARS-CoV-2 antigen (eg, spike); VH-IHI pairs with VL-IHI; VH-IHG pairs with VL-IHG, etc. VH-IHI (SEQ ID NO: 295):

CAGGTGCAGCTGGTGCAGTCTGGGGCTGAGGTGAAGAAGCCTGGGGCCTC
AGTGAAGGTCTCCTGCAAGGCTTCTGGATACATCTTCACCGGCTACTATA
TGCACTGGGTGCGACAGGCCCCTGGACAGGGGCTTGAGTGGATGGGATGG
ATCAACCCTAACAGTGGTGGCGCAAACTATGCACAGAAGTTTCAGGGCAG
GGTCACCCTGACCAGGGACACGTCCATCACCACAGTCTACATGGAACTGA
GCAGGCTGAGATTTGACGACACGGCCGTGTATTACTGTGCGAGAGGATCC
CGGTATGACTGGAACCAGAACAACTGGTTCGACCCCTGGGGCCAGGGAAC
CCTGGTCACCGTCTCCTCA

VL-IHI (SEQ ID NO: 296):

CAGTCTGCCCTGACTCAGCCTGCCTCCGTGTCTGGGTCTCCTGGACAGTC
GATCACCATCTCCTGCACTGGAACCAGCAGTGACGTTGGTACTTATAACT
ATGTCTCCTGGTACCAACAACACCCAGGCAAAGCCCCCAAACTCATGATT
TTTGATGTCAGTAATCGGCCCTCAGGGGTTTCTGATCGCTTCTCTGGCTC
CAAGTCTGGCAACACGGCCTCCCTGACCATCTCTGGGCTCCAGGCTGAGG
ACGAGGCTGATTATTACTGCAGCTCATTTACAACCAGCAGCACTGTGGTT
TTCGGCGGAGGGACCAAGCTGACCGTCCTA

VH-IHG (SEQ ID NO: 297):

CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTATGCTA
TGTACTGGGTCCGCCAGGCTCCAGGCAAGGGGCTGGAGTGGGTGGCAGTT
ATATCATATGATGGAAGTAATAAATACTATGCAGACTCCGTGAAGGGCCG
ATTCACCATCTCCAGAGACAATTCCAAGAACACGCTGTATCTGCAAATGA
ACAGCCTGAGAACTGAGGACACGGCTGTGTATTACTGTGCGAGTGGCTCC
GACTACGGTGACTACTTATTGGTTTACTGGGGCCAGGGAACCCTGGTCAC
CGTCTCCTCA

VL-IHG (SEQ ID NO: 298):

CAGTCTGCCCTGACTCAGCCTGCCTCCGTGTCTGGGTCTCCTGGACAGTC
GATCACCATCTCCTGCACTGGAACCAGCAGTGACGTTGGTGGTTATAACT
ATGTCTCCTGGTACCAACAACACCCAGGCAAAGCCCCCAAACTCATGATT
TATGATGTCAGTAAGCGGCCCTCAGGGGTTTCTAATCGCTTCTCTGGCTC
CAAGTCTGGCAACACGGCCTCCCTGACCATCTCTGGGCTCCAGTCTGAGG
ACGAGGCTGATTATTACTGCAACTCTTTGACAAGCATCAGCACTTGGGTG
TTCGGCGGAGGGACCAAGCTGACCGTCCTA

VH-ICC (SEQ ID NO: 299):

CAGGTGCAGCTGGTGGAGTCTGGGGGAGGCTTGGTCAAGCCTGGAGGGTC
CCTGAGACTCTCCTGTGCAGCCTCTGGATTCACCTTCAGTGACTACTACA
TGAGCTGGATCCGCCAGGCTCCAGGGAAGGGGCTGGAGTGGGTTTCATAC
ATTACTTATAGTGGTAGTACCATATACTACGCAGACTCTGTGAAGGGCCG
ATTCACCATCTCCAGGGACAACGCCAAGAGCTCACTGTATCTGCAAATGA
ACAGCCTGAGAGCCGAGGACACGGCCGTGTATTACTGTGCGAGAGATCGC
GGTACAACTATGGTCCCCTTTGACTACTGGGGCCAGGGAACCCTGGTCAC
CGTCTCCTCA

VL-ICC (SEQ ID NO: 300):

GACATCCAGATGACCCAGTCTCCATCCTCCCTGTCTGCATCTGTAGGAGA
CAGAGTCACCATCACTTGCCAGGCGAGTCAGGACATTACCAACTATTTAA
ATTGGTATCAGCAGAAACCAGGGAAAGCCCCTAAGCTCCTGATCTACGCT
GCATCCAATTTGGAAACAGGGGTCCCATCAAGGTTCAGTGGAAGTGGATC
TGGGACAGATTTTACTTTCACCATCAGCGGCCTGCAGCCTGAAGATATTG
CAACATATTACTGTCAACAGTATGATAATCTCCCTCTCACTTTCGGCGGA
GGGACCAAGGTGGAGATCAAA

VH-ICD (SEQ ID NO: 301):

GAGGTGCAGCTGGTGGAGTCTGGGGGAGGCTTGGTAAAGCCTGGGGGGTC
CCTTAGACTCTCCTGTGCAGCCTCTGGAATCACTTTCAGTAACGCCTGGA
TGAGTTGGGTCCGCCAGGCTCCAGGGAAGGGGCTGGAGTGGGTTGGCCGT
ATTAAAAGCAAAACTGATGGTGGGACAACAGACTACGCCGCACCCGTGAA
AGGCAGATTCACCATCTCAAGAGATGATTCAAAAAACACGCTGTATCTAC
AAATGAACAGCCTGAAAACCGAGGACACAGCCGTGTATTACTGTACCACA
GCGAGGTGGGACTGGTACTTCGATCTCTGGGGCCGTGGCACCCTGGTCAC
TGTCTCCTCA

VL-ICD (SEQ ID NO: 302):

GACATCCAGATGACCCAGTCTCCATCCTCCCTGTCTGCATCTGTAGGAGA
CAGAGTCACCATCACTTGCCAGGCGAGTCAGGACATTTGGAATTATATAA
ATTGGTATCAGCAGAAACCAGGGAAGGCCCCTAAGCTCCTGATCTACGAT
GCATCCAATTTGAAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGCATGATGATCTCCCTCCGACCTTCGGCCAA
GGGACCAAGGTGGAAATCAAA

VH-IGG (SEQ ID NO: 303):

CAGGTGCAGCTGGTGCAGTCTGGGGCTGAGGTGAAGAAGCCTGGGTCCTC
GGTGAAGGTCTCCTGCAAGGCTTCTGGAGGCACCTTCAGCAGCTATGCTA
TCACCTGGGTGCGACAGGCCCCTGGACAAGGGCTTGAGTGGATGGGAAGG
ATCATCCCTATGTTTGGTATAGCAAACTACGCACAGAAGTTCCAGGGCAG
AGTCACGATTACCGCGGACAAATCCACGAGCACAGCCTACATGGAGGTGA
GCAGCCTGAGATCTGAGGACACGGCCGTGTATTACTGTGCGAGAACACCG
TTTTACTATGATAGTAGTGGTTATTACCTTGACTACTGGGGCCAGGGAAC
CCTGGTCACCGTCTCCTCA

VL-IGG (SEQ ID NO: 304):

GAAATTGTGTTGACGCAGTCTCCAGGCACCCTGTCTTTGTCTCCAGGGGA
AAGAGCCACCCTCTCTTGCAGGGCCAGTCAGAGTGTTAGCAGCAACTACT
TAGCCTGGTACCAGCAGAGTCCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTTTATTACTGTCAGCAGTATGTTAGGTCACCCCGGACGTTCGGC
CAAGGGACCAAGGTGGAAATCAAA

VH-IFD (SEQ ID NO: 305):

CAGGTGCAGCTGCAGGAGTCGGGCCCAGGACTGGTGAAGCCTTCGGAGAC
CCTGTCCCTCACCTGCACTGTCTCTGGTGGCTCCATCAATAATTACTACT
GGATCTGGATCCGGCAGCCCCCAGGGAAGGGACTGGAGTGGATTGGGTAT
ATCTATCACAGTGGGAGCACCAACTACAACCCCTCCCTCAAGAGTCGAGT
CACCATATCAGTAGACACGTCCAAGAACCAATTCTCCCTGAAGGTGAGCT
CTGTGACCGCTGCGGACACGGCCGTGTATTACTGTGCGAATATGGTTCGG
GGAGTTTATGAAGATGACTACTGGGGCCAGGGAACCCTGGTCACCGTCTC
CTCA

VL-IFD (SEQ ID NO: 306):

GACATCCAGATGACCCAGTCTCCATCCTCCCTGTCTGCATCTGTAGGAGA
CAGAGTCACCATCACTTGCCGGGCAAGTCAGAGCATTAGCAACTATTTAA
ATTGGTATCAGCAGAAACCAGGGAAAGCCCCTAAGCTCCTGATCTTTGCT
GCCTCCAGTTTGCGAAGTGGGGTCCCATCAAGGTTCAGTGGCAGTGGATC
TGGGACAGATTTCACTCTCACCATCAGCAGTCTGCACCCTGAAGATTTTG
CAACTTACTACTGTCAACAGAATTACAATACCCCCCTCACTTTCGGCCCT
GGGACCAAAGTGGATATCAAA

VH-IED (SEQ ID NO: 308):

GAGGTGCAGCTGGTGGAGTCTGGGGGAGGCTTGGTCCAGCCTGGGGGGTC
ACTGAGACTCTCCTGTGCAGCCTCTGGAATCACCGTCAGTAGCAACTACA
TGAGCTGGGTCCGCCAGGCTCCAGGGAAGGGGCTGGAGTGGGTCTCACTT
ATTTATAGTGGTGGTAGCACATACTACGCAGACTCCGTGAAGGGCAGATT
CACCATCTCCAGAGACAATTCCAAGAACACGCTGTATCTTCAAATGAACA
GCCTGAGAGTCGAGGACACGGCTGTGTATTACTGTGCGAGAGATCACGGT
ATGGCAGCAGCGGGGTATAATTACTGGGGCCAGGGAACCCTGGTCACCGT
CTCCTCA

VL-IED (SEQ ID NO: 309):

GACATCCAGATGACCCAGTCTCCATCCTCCCTGTCTGCGTCTGTAGGAGA
CAGAGTCACCATCACTTGCCAGGCGAGTCAGGACATTAACAAATATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAACAGCCTGCAGCCTGAAGATTTTG
CAACATATTTCTGTCAACAGTATGATAATCTCCCTCCAGCGTTCGGCCAA
GGGACCAAGGTGGAAATCAAA

VH-IHD (SEQ ID NO: 310):

GAGGTGCAGCTGGTGGAGTCTGGGGGAGGCTTGGTCCAGCCTGGGGGGTC
CCTGAGACTCTCCTGTGCAGCCTCTGGACTCACCGTCAATCGCAACTACA
TGAGCTGGGTCCGCCAGGCTCCAGGGAAGGGGCTGGAGTGGGTCTCACTT
ATTTATAGCGGTGGTAGCACATACTACGCAGACTCCGTTAAGGGCCGATT
CACCATCTCCAGAGACAATTCCAAGAACACGCTGTATCTTCAAATGAACA
GCCTGAGACTTGAGGACACGGCTGTGTATTACTGTGCGAGAGGTGAACTG
GGGATCCCCTACGGTATGGACGTCTGGGGCCAAGGGACCACGGTCACCGT
CTCCTCA

VL-IHD (SEQ ID NO: 311):

CAGTCTGTGTTGACGCAGCCGCCCTCAGTGTCTGCGGCCCCAGGACAGAA
GGTCACCATCTCCTGCTCTGGAAGCAGCTCCAACATTGGGAATAATTATG
TATCCTGGTACCAGCAGCTCCCAGGAACAGCCCCCAAACTCCTCATTTAT
GACAATAATAAGCGACCCTCAGGGATTCCTGACCGCTTCTCTGGCTCCAA
GTCTGGCACGTCAGCCACCCTGGGCATCACCGGACTCCAGACTGGGGACG
AGGCCGATTATTACTGCGGAACATGGGATAGCAGCCTGAGTGCTGGGGTG
TTCGGCGGAGGGACCAAGCTGACCGTCCTA

VH-IHF (SEQ ID NO: 312):

GAGGTGCAGCTGGTGGAGTCTGGGGGAGGCTTGGTCCAGCCTGGGGGGTC
CCTGAGACTCTCCTGTGCAGCCTCTGAATTCACCGTCAGTAGCAACTACA
TGAGCTGGGTCCGCCAGGCTCCAGGGAAGGGGCTAGAGTGGGTCTCAGTT
ATTTATAGCGGTGGTAGCACATTTTACGCAGACTCCGTGAAGGGCAGATT
CACCATCTCCAGAGACAATTCCAAGAACACGTTGTATCTTCAAATGAACA
GTCTGAGAGCCGAGGACACGGCTGTATATTACTGTGCGAGAGCTCTTCCC
TACGGTGACTTGCATTTTGACTACTGGGGCCAGGGAACCCTGGTCACCGT
CTCCTCA

VL-IHF (SEQ ID NO: 313):

CAGTCTGTGCTGACGCAGCCGCCCTCAGTGTCTGGGGCCCCAGGGCAGAG
GGTCACCATCTCCTGCACTGGGAGCAGCTCCAACATCGGGACAGGTTATG
ATGTACACTGGTACCAGCAGCTTCCAGGAACAGCCCCCAAACTCCTCATC
TATGGTAACACCAATCGGCCCTCAGGGGTCCCTGACCGATTCTCTGGCTC
CAAGTCTGGCACCTCAGCCTCCCTGGCCATCACTGGGCTCCAGGCTGAGG
ATGAGGCTGATTATTACTGCCAGTCCTATGACAGCAGCCTGAGTGACTCT
TATGTCTTCGGAACTGGGACCAAGGTCACCGTCCTA

TABLE 22

Examples of affinity tags and their amino acid sequence and ligand

| | | |
|---|---|---|
| Affinity Tag | Tag Sequence | Ligand |
| Poly His (6xHis) (SEQ ID NO: 314) | HHHHHH | Ni²⁺ NTA, CO²⁺CMA |
| Poly His (8xHis) (SEQ ID NO: 315) | HHHHHHHH | |
| Poly His (10xHis) (SEQ ID NO: 316) | HHHHHHHHHH | |
| HAT (SEQ ID NO: 317) | KDHLIHVHLEEHAHAHNK | CO²⁺ CMA |
| FLAG (SEQ ID NO: 318) | DYKDDDDK | mAb M1, M2 |
| c-myc (SEQ ID NO: 319) | EQKLISEEDL | mAb 9E10 |
| T7 (SEQ ID NO: 320) | MASMTGGQQMG | Anti-T7 9E10 |
| S-tag (SEQ ID NO: 321) | KETAAAKFERGHMDS | S-protein |
| Calmodulin-binding protein (SEQ ID NO: 322) | KRRWKKNFIAVSAANRFKKISSSGAL | S-protein Calmodulin |
| Bio tag (SEQ ID NO: 323) | LGIFEAMKMEWR | Streptavidin/Avidin |
| Strep-tag (SEQ ID NO: 324) | SAWRHPQFGG | Streptavidin |
| Strep-tag II (SEQ ID NO: 325) | WHPQFEK | Streptavidin-Tactin |
| Avi tag (SEQ ID NO: 326) | GLNDIFEAQKIEWHE | Streptavidin/Avidin |
| Nanotag (SEQ ID NO: 327) | DVEAWLGAR | Streptavidin/Avidin |
| Poly-Arg (R) | 5-16 amino acids | Anionic resins |
| Poly-Asp (D) | 5-16 amino acids | Cationic resins |
| Poly-Cys (C) | 2-10 amino acids | Thiopropyl Sepharose |
| Poly-Phe (F) | 5-16 amino acids | IEC |
| Glu (E) | 1 amino acid | Cationic resins |

Table 23. Amino acid sequences of anti-SARS-CoV/anti-SARS-CoV-2 in various formats for capturing and/or neutralizing SARS-CoV, SARS-CoV-2 and other related coronavirus strains. Each of the sequences outlined in this table can be further generated with or without a N- or C-terminus affinity tag as outlined in Table 22. The tetramerization domain (TD) is preferably TD from p53 but not limited by it. CR3006, CR3013, CR3014 and CR3022 are example antibody sequences for anti-SARS-CoV and CoV-2 strains. Other anti-SARS-CoV/anti-SARS-CoV-2 could be composed of single domain antibodies (dAb) such as those from camelid. Examples of anti-SARS-CoV/anti-SARS-CoV-2 dAb include QB-DD and QB-GB formatted into different Quad formats as shown in FIGS. 6A-D). Additional examples of anti-SARS-CoV/anti-SARS-CoV-2 Quads could include peptides that target and/or inhibit virus-host membrane fusion such as EK1C4 formatted into different Quad formats as shown in FIGS. 6A-D. In further examples, other anti-SARS-CoV and CoV-2 IgG antibodies can be formatted into the different formats exemplified this table or schematically shown in FIGS. 4A-C.

The sequences of binding domains (eg, VH and VL) for use in the present invention are underlined and are intended to be read as written (ie, as part of the full sequence including non-underlined part) or alternatively without the futher underlined sequence.

In an embodiment, the polypeptide of the invention comprises a sequence that is shown in bold in this Table 23. In an embodiment, the polypeptide of the invention comprises, in N- to C-terminal direction, a binding domain or peptide; and a sequence that is shown in bold in this Table 23. The binding domain may be any binding domain disclosed herein, eg, a VH, VL, VHH, dAb or scFv.

The present disclosure further comprises each underlined sequence without the following bold sequence, eg, to provide a binding site or domain for use in any polypeptide or mutimer of the invention herein. The present disclosure further comprises each bold sequence without the immediately preceding underlined sequence, eg, to for use in any polypeptide or mutimer of the invention herein (such as as part of a polypeptide comprising a binding domain or peptide immediately preceding the bold sequence).

| | |
|---|---|
| Name | Sequence |
| CR3006 VH IgG (SEQ ID NO: 328) | EVQLVESGGGLVQPGGSLRLSCAASGFTFSGYPMHWVRQAPGKGLEWVA VISYDGSNKYYADSVKGRFTISRDNSKNTLYLQMNSLRAEDTAVYYCAK DGSPRTPSFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKS LSLSPG |
| CR3006 VL kappa (SEQ ID NO: 329) | DIQMTQSPHSLSASVGDRVTITCRASQGISNYLAWYQQKPGKVPKLLIY AASTLQSGVPSRFSGSGSGTDFTLTISSLQPEDVGVYYCQQRFRTPVTF GQGTKLEIK RTVAAPSVFIFPPSDEQLKSGTASWCLLNNFYPREAKVQ WKVDNALQSGNSQESVTEQDSKDSTYSLSSTLTLSKADYEKHKVYACEV THQGLSSPVTKSFNRGEC |
| CR3006 VH-Fab-TD (SEQ ID NO: 330) | EVQLVESGGGLVQPGGSLRLSCAASGFTFSGYPMHWVRQAPGKGLEWVA VISYDGSNKYYADSVKGRFTISRDNSKNTLYLQMNSLRAEDTAVYYCAK DGSPRTPSFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTKKKPLDGEYFTLQIRGRER FEMFRELNEALELKDAQAGKEPG |
| CR3006 VH Fab monomeric Ig TD (SEQ ID NO: 331) | EVQLVESGGGLVQPGGSLRLSCAASGFTFSGYPMHWVRQAPGKGLEWVA VISYDGSNKYYADSVKGRFTISRDNSKNTLYLQMNSLRAEDTAVYYCAK DGSPRTPSFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTAPELLGGPSVFLFPPKPKD TLMISRTPEVTCVWDVSHEDPEVKFNWYVDGVEVHNAKTKPREEQYNS TYRVVSVLTVLHQDWLNGKEYKCKVSNKALPAPIEKTISKAKGQPREPQ VYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSP GKKKKPLDGEYFTLQIRGRERFEMFRELNEALELKDAQAGKEPG |
| CR3006 VH Fab Ig TD (SEQ ID NO: 332) | EVQLVESGGGLVQPGGSLRLSCAASGFTFSGYPMHWVRQAPGKGLEWVA VISYDGSNKYYADSVKGRFTISRDNSKNTLYLQMNSLRAEDTAVYYCAK DGSPRTPSFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKS LSLSPGKKKKPLDGEYFTLQIRGRERFEMFRELNEALELKDAQAGKEPG |
| CR3006 scFv | EVQLVESGGGLVQPGGSLRLSCAASGFTFSGYPMHWVRQAPGKGLEWVA VISYDGSNKYYADSVKGRFTISRDNSKNTLYLQMNSLRAEDTAVYYCAK DGSPRTPSFDYWGQGTLVTVGGGGSGGGGSGGGGSDIQMTQSPHSLSAS VGDRVTITCRASQGISNYLAWYQQKPGKVPKLLIYAASTLQSGVPSRFS |
| | GSGSGTDFTLTISSLQPEDVGVYYCQQRFRTPVTFGQGTKLEIK |
| CR3006 scFv-TD (SEQ ID NO: 333) | EVQLVESGGGLVQPGGSLRLSCAASGFTFSGYPMHWVRQAPGKGLEWVAVISYDGSNKYYADSVKGRFTISRDNSKNTLYLQMNSLRAEDTAVYYCAKDGSPRTPSFDYWGQGTLVTVGGGGSGGGGSGGGGSDIQMTQSPHSLSAS VGDRVTITCRASQGISNYLAWYQQKPGKVPKLLIYAASTLQSGVPSRFS GSGSGTDFTLTISSLQPEDVGVYYCQQRFRTPVTFGQGTKLEIK KKKPL DGEYFTLQIRGRERFEMFRELNEALELKDAQAGKEPG |
| CR3006 scFv monomeric Ig-TD (SEQ ID NO: 334) | EVQLVESGGGLVQPGGSLRLSCAASGFTFSGYPMHWVRQAPGKGLEWVA VISYDGSNKYYADSVKGRFTISRDNSKNTLYLQMNSLRAEDTAVYYCAK DGSPRTPSFDYWGQGTLVTV GGGGSGGGGSGGGGSDIQMTQSPHSLSAS VGDRVTITCRASQGISNYLAWYQQKPGKVPKLLIYAASTLQSGVPSRFS GSGSGTDFTLTISSLQPEDVGVYYCQQRFRTPVTFGQGTKLEIKEPKSC DKTHTAPELLGGPSVFLFPPKPKDTLMISRTPEVTCVWDVSHEDPEVK FNWYVDGVEVHNAKTKPREEQYNSTYRWSVLTVLHQDWLNGKEYKCKV SNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGN VFSCSVMHEALHNHYTQKSLSLSPGKKKKPLDGEYFTLQIRGRERFEMF RELNEALELKDAQAGKEPG |
| CR3013 VH IgG (SEQ ID NO: 335) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKS LSLSPG |
| CR3013 VL kappa (SEQ ID NO: 336) | ELTQSPSSLSASVGDRVTITCRASQSISSYLNWYQQKPGKAPKLLIYAA SSLQSGVPSRFSGSGSGTDFTLTISSLQPEDFATYYCQQSYSTPPTFGQ GTKVEIK RTVAAPSVFIFPPSDEQLKSGTASVVCLLNNFYPREAKVQWK VDNALQSGNSQESVTEQDSKDSTYSLSSTLTLSKADYEKHKVYACEVTH QGLSSPVTKSFNRGEC |
| CR3013 VH-Fab-TD (SEQ ID NO: 337) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTKKKPLDGEYFTLQIRGRER |
| | FEMFRELNEALELKDAQAGKEPG |
| CR3013 VH Fab monomeric Ig TD (SEQ ID NO: 338) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTAPELLGGPSVFLFPPKPKD TLMISRTPEVTCVWDVSHEDPEVKFNWYVDGVEVHNAKTKPREEQYNS TYRVVSVLTVLHQDWLNGKEYKCKVSNKALPAPIEKTISKAKGQPREPQ VYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSP GKKKKPLDGEYFTLQIRGRERFEMFRELNEALELKDAQAGKEPG |
| CR3013 VH Fab Ig TD (SEQ ID NO: 339) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKS LSLSPGKKKKPLDGEYFTLQIRGRERFEMFRELNEALELKDAQAGKEPG |
| CR3013 scFv (SEQ ID NO: 340) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTVGGGGSGGGGSGGGGSELTQSPSSLSASVG DRVTITCRASQSISSYLNWYQQKPGKAPKLLIYAASSLQSGVPSRFSGS GSGTDFTLTISSLQPEDFATYYCQQSYSTPPTFGQGTKVEIK |
| CR3013 scFv-TD (SEQ ID NO: 341) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTVGGGGSGGGGSGGGGSELTQSPSSLSASVG DRVTITCRASQSISSYLNWYQQKPGKAPKLLIYAASSLQSGVPSRFSGS GSGTDFTLTISSLQPEDFATYYCQQSYSTPPTFGQGTKVEIKKKKPLDG EYFTLQIRGRERFEMFRELNEALELKDAQAGKEPG |
| CR3013 scFv monomeric | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC AKGLTPLYFDYWGQGTLVTVGGGGSGGGGSGGGGSELTQSPSSLSASVG |
| Ig-TD (SEQ ID NO: 342) | DRVTITCRASQSISSYLNWYQQKPGKAPKLLIYAASSLQSGVPSRFSGS GSGTDFTLTISSLQPEDFATYYCQQSYSTPPTFGQGTKVEIK EPKSCDK THTAPELLGGPSVFLFPPKPKDTLMISRTPEVTCVWDVSHEDPEVKFN WYVDGVEVHNAKTKPREEQYNSTYRVVSVLTVLHQDWLNGKEYKCKVSN KALPAPIEKTISKAKGQPREPQVYTLPPSRDELTKNQVSLTCLVKGFYP SDIAVEWESNGQPENNYKTTPPVLDSDGSFFLYSKLTVDKSRWQQGNVF SCSVMHEALHNHYTQKSLSLSPGKKKKPLDGEYFTLQIRGRERFEMFRE LNEALELKDAQAGKEPG |
| CR3014 VH IgG (SEQ ID NO: 343) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKS LSLSPG |
| CR3014 VL kappa (SEQ ID NO: 344) | ELTQSPSSLSASVGDRVTITCRASQSISSYLNWYQQKPGKAPKLLIYAA SSLQSGVPSRFSGSGSGTDFTLTISSLQPEDFATYYCQQSYSTPPTFGQ GTKVEIK RTVAAPSVFIFPPSDEQLKSGTASWCLLNNFYPREAKVQWK VDNALQSGNSQESVTEQDSKDSTYSLSSTLTLSKADYEKHKVYACEVTH QGLSSPVTKSFNRGEC |
| CR3014 VH-Fab-TD (SEQ ID NO: 345) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTKKKPLDGEYFTLQIRGRER FEMFRELNEALELKDAQAGKEPG |
| CR3014 VH Fab monomeric Ig TD (SEQ ID NO: 346) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTAPELLGGPSVFLFPPKPKD TLMISRTPEVTCVWDVSHEDPEVKFNWYVDGVEVHNAKTKPREEQYNS TYRVVSVLTVLHQDWLNGKEYKCKVSNKALPAPIEKTISKAKGQPREPQ VYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSP |
| | GKKKKPLDGEYFTLQIRGRERFEMFRELNEALELKDAQAGKEPG |
| CR3014 VH Fab Ig TD (SEQ ID NO: 347) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTV ASTKGPSVFPLAPSSKSTSGGTAALGCLV KDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKS LSLSPGKKKKPLDGEYFTLQIRGRERFEMFRELNEALELKDAQAGKEPG |
| CR3014 scFv (SEQ ID NO: 348) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTVGGGGSGGGGSGGGGSELTQSPSSLSASVG DRVTITCRASQSISSYLNWYQQKPGKAPKLLIYAASSLQSGVPSRFSGS GSGTDFTLTISSLQPEDFATYYCQQSYSTPPTFGQGTKVEIK |
| CR3014 scFv-TD (SEQ ID NO: 349) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTVGGGGSGGGGSGGGGSELTQSPSSLSASVG DRVTITCRASQSISSYLNWYQQKPGKAPKLLIYAASSLQSGVPSRFSGS GSGTDFTLTISSLQPEDFATYYCQQSYSTPPTFGQGTKVEIK KKKPLDG EYFTLQIRGRERFEMFRELNEALELKDAQAGKEPG |
| CR3014 scFv monomeric Ig-TD (SEQ ID NO: 350) | EVQLVESGGGLVQPGGSLRLSCAASGFTFSDHYMDWVRQAPGKGLEWVG RTRNKANSYTTEYAASVKGRFTISRDDSKNSLYLQMNSLKTEDTAVYYC ARGISPFYFDYWGQGTLVTVGGGGSGGGGSGGGGSELTQSPSSLSASVG DRVTITCRASQSISSYLNWYQQKPGKAPKLLIYAASSLQSGVPSRFSGS GSGTDFTLTISSLQPEDFATYYCQQSYSTPPTFGQGTKVEIK EPKSCDK THTAPELLGGPSVFLFPPKPKDTLMISRTPEVTCVWDVSHEDPEVKFN WYVDGVEVHNAKTKPREEQYNSTYRVVSVLTVLHQDWLNGKEYKCKVSN KALPAPIEKTISKAKGQPREPQVYTLPPSRDELTKNQVSLTCLVKGFYP SDIAVEWESNGQPENNYKTTPPVLDSDGSFFLYSKLTVDKSRWQQGNVF SCSVMHEALHNHYTQKSLSLSPGKKKKPLDGEYFTLQIRGRERFEMFRE LNEALELKDAQAGKEPG |
| CR3022 VH IgG (SEQ ID NO: 351) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTV ASTKGPSVFPLAPSSKSTSGGTAALGCLVK DYFPEPVTVSWNSGALTSGVHTFPAVLQSSGLYSLSSVVTVPSSSLGTQ TYICNVNHKPSNTKVDKKVEPKSCDKTHTCPPCPAPELLGGPSVFLFPP KPKDTLMISRTPEVTCVWDVSHEDPEVKFNWYVDGVEVHNAKTKPREE QYNSTYRVVSVLTVLHQDWLNGKEYKCKVSNKALPAPIEKTISKAKGQP REPQVYTLPPSRDELTKNQVSLTCLVKGFYPSDIAVEWESNGQPENNYK TTPPVLDSDGSFFLYSKLTVDKSRWQQGNVFSCSVMHEALHNHYTQKSL SLSPG |
| CR3022 VL kappa (SEQ ID NO: 352) | DIQLTQSPDSLAVSLGERATINCKSSQSVLYSSINKNYLAWYQQKPGQP PKLLIYWASTRESGVPDRFSGSGSGTDFTLTISSLQAEDVAVYYCQQYY STPYTFGQGTKVEIK RTVAAPSVFIFPPSDEQLKSGTASWCLLNNFYP REAKVQWKVDNALQSGNSQESVTEQDSKDSTYSLSSTLTLSKADYEKHK VYACEVTHQGLSSPVTKSFNRGEC |
| CR3022 VH-Fab-TD (SEQ ID NO: 353) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTV ASTKGPSVFPLAPSSKSTSGGTAALGCLVK DYFPEPVTVSWNSGALTSGVHTFPAVLQSSGLYSLSSVVTVPSSSLGTQ TYICNVNHKPSNTKVDKKVEPKSCDKTHTKKKPLDGEYFTLQIRGRERF EMFRELNEALELKDAQAGKEPG |
| CR3022 VH Fab monomeric Ig TD (SEQ ID NO: 354) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTV ASTKGPSVFPLAPSSKSTSGGTAALGCLVK DYFPEPVTVSWNSGALTSGVHTFPAVLQSSGLYSLSSVVTVPSSSLGTQ TYICNVNHKPSNTKVDKKVEPKSCDKTHTAPELLGGPSVFLFPPKPKDT LMISRTPEVTCVVVDVSHEDPEVKFNWYVDGVEVHNAKTKPREEQYNST YRVVSVLTVLHQDWLNGKEYKCKVSNKALPAPIEKTISKAKGQPREPQV YTLPPSRDELTKNQVSLTCLVKGFYPSDIAVEWESNGQPENNYKTTPPV LDSDGSFFLYSKLTVDKSRWQQGNVFSCSVMHEALHNHYTQKSLSLSPG KKKKPLDGEYFTLQIRGRERFEMFRELNEALELKDAQAGKEPG |
| CR3022 VH Fab Ig TD (SEQ ID NO: 355) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTV ASTKGPSVFPLAPSSKSTSGGTAALGCLVK DYFPEPVTVSWNSGALTSGVHTFPAVLQSSGLYSLSSVVTVPSSSLGTQ TYICNVNHKPSNTKVDKKVEPKSCDKTHTCPPCPAPELLGGPSVFLFPP KPKDTLMISRTPEVTCVWDVSHEDPEVKFNWYVDGVEVHNAKTKPREE QYNSTYRVVSVLTVLHQDWLNGKEYKCKVSNKALPAPIEKTISKAKGQP REPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSL SLSPGKKKKPLDGEYFTLQIRGRERFEMFRELNEALELKDAQAGKEPG |
| CR3022 scFv (SEQ ID NO: 356) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTVGGGGSGGGGSGGGGSDIQLTQSPDSLAVSL GERATINCKSSQSVLYSSINKNYLAWYQQKPGQPPKLLIYWASTRESGV PDRFSGSGSGTDFTLTISSLQAEDVAVYYCQQYSTPYTFGQGTKVEIK |
| CR3022 scFv-TD (SEQ ID NO: 357) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTVGGGGSGGGGSGGGGSDIQLTQSPDSLAVSL GERATINCKSSQSVLYSSINKNYLAWYQQKPGQPPKLLIYWASTRESGV PDRFSGSGSGTDFTLTISSLQAEDVAVYYCQQYYSTPYTFGQGTKVEIK KKKPLDGEYFTLQIRGRERFEMFRELNEALELKDAQAGKEPG |
| CR3022 scFv monomeric Ig-TD (SEQ ID NO: 358) | QMQLVQSGTEVKKPGESLKISCKGSGYGFITYWIGWVRQMPGKGLEWMG IIYPGDSETRYSPSFQGQVTISADKSINTAYLQWSSLKASDTAIYYCAG GSGISTPMDVWGQGTTVTVGGGGSGGGGSGGGGSDIQLTQSPDSLAVSL GERATINCKSSQSVLYSSINKNYLAWYQQKPGQPPKLLIYWASTRESGV PDRFSGSGSGTDFTLTISSLQAEDVAVYYCQQYSTPYTFGQGTKVEIK E PKSCDKTHTAPELLGGPSVFLFPPKPKDTLMISRTPEVTCVVVDVSHED PEVKFNWYVDGVEVHNAKTKPREEQYNSTYRWSVLTVLHQDWLNGKEY KCKVSNKALPAPIEKTISKAKGQPREPQVYTLPPSRDELTKNQVSLTCL VKGFYPSDIAVEWESNGQPENNYKTTPPVLDSDGSFFLYSKLTVDKSRW QQGNVFSCSVMHEALHNHYTQKSLSLSPGKKKKPLDGEYFTLQIRGRER FEMFRELNEALELKDAQAGKEPG |
| QB-DD (SEQ ID NO: 359) | QVQLQESGGGLVQTGGSLRLSCAASGSDFSSYAMAWFRQAPG |
| | KEREFVASISRRSTNTYYRNSVKGRFTISRDNAKNTAWLQMN |
| | SLKPEDTAVYYCAADRARYGSSWYESLAYLEVWGQGTQVTVSS |
| QB-GB (SEQ ID NO: 307) | QVQLQESGGGLVQAGGSLRLSCAASGRTFSEYAMGWFRQAPG |
| | KEREFVATISWSGGSTYYTDSVKGRFTISRDNAKNTVYLQMN |
| | SLKPDDTAVYYCAAAGLGTVVSEWDYDYDYWGQGTQVTVSS |
| EK1C4 (Peptide) | SLDQINVTFLDLEYEMKKLEEAIKKLEESYIDLKEL |
| (SEQ ID NO: 360) | |

TABLE 23 Continued

Amino acid sequence of Quad polypeptide monomers in different formats, all containing VHH QB-GB (QB-GB is the sequence that is not in bold in Q177 to 186)

| | |
|---|---|
| Name | Sequence |
| Q177 (SEQ ID NO: 361) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSS GGGGSGGGGSKKKPLDGEYFTLQIRGRE RFEMFRELNEALELKDAQAGKEPG |
| Q178 (SEQ ID NO: 362) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSEPKSCDKTHTCPPCPAPE LLGGPSVFLFPPKPKDTLMISRTPEVTCVWDVSHEDPEVKFNWYVDGVEV HNAKTKPREEQYNSTYRVVSVLTVLHQDWLNGKEYKCKVSNKALPAPIEKT ISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYT QKSLSLSPGK |
| Q179 (SEQ ID NO: 363) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSEPKSCDKTHTCPPCPAPE LLGGPSVFLFPPKPKDTLMISRTPEVTCVWDVSHEDPEVKFNWYVDGVEV HNAKTKPREEQYNSTYRVVSVLTVLHQDWLNGKEYKCKVSNKALPAPIEKT ISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYT QKSLSLSPGKKKKPLDGEYFTLQIRGRERFEMFRELNEALELKDAQAGKEP G |
| Q180 (SEQ ID NO: 364) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSASTKGPSVFPLAPSSKST SGGTAALGCLVKDYFPEPVTVSWNSGALTSGVHTFPAVLQSSGLYSLSSW TVPSSSLGTQTYICNVNHKPSNTKVDKKVEPKSCDKTHTCPPCPAPELLGG PSVFLFPPKPKDTLMISRTPEVTCVWDVSHEDPEVKFNWYVDGVEVHNAK TKPREEQYNSTYRVVSVLTVLHQDWLNGKEYKCKVSNKALPAPIEKTISKA KGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSL SLSPGK |
| Q181 (SEQ ID NO: | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSASTKGPSVFPLAPSSKST |
| 365) | SGGTAALGCLVKDYFPEPVTVSWNSGALTSGVHTFPAVLQSSGLYSLSSW TVPSSSLGTQTYICNVNHKPSNTKVDKKVEPKSCDKTHTCPPCPAPELLGG PSVFLFPPKPKDTLMISRTPEVTCVWDVSHEDPEVKFNWYVDGVEVHNAK TKPREEQYNSTYRVVSVLTVLHQDWLNGKEYKCKVSNKALPAPIEKTISKA KGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSL SLSPGKKKKPLDGEYFTLQIRGRERFEMFRELNEALELKDAQAGKEPG |
| Q182 (SEQ ID NO: 286) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSKRTVAAPSVFIFPPSDEQ LKSGTASVVCLLNNFYPREAKVQWKVDNALQSGNSQESVTEQDSKDSTYSL SSTLTLSKADYEKHKVYACEVTHQGLSSPVTKSFNRGEC |
| Q183 (SEQ ID NO: 366) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSASTKGPSVFPLAPSSKST SGGTAALGCLVKDYFPEPVTVSWNSGALTSGVHTFPAVLQSSGLYSLSSW TVPSSSLGTQTYICNVNHKPSNTKVDKKVEPKSCDKTHTKKKPLDGEYFTL QIRGRERFEMFRELNEALELKDAQAGKEPG |
| Q184 (SEQ ID NO: 367) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSASTKGPSVFPLAPSSKST SGGTAALGCLVKDYFPEPVTVSWNSGALTSGVHTFPAVLQSSGLYSLSSW TVPSSSLGTQTYICNVNHKPSNTKVDKKVEPKSCDKTHTAPELLGGPSVFL FPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSLSPG KKKKPWGEYFTLQIRGRERFEMFRELNEALELKDAQAGKEPG |
| Q185 (SEQ ID NO: 368) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSEPKSCDKTHTCPPCPAPE LLGGKKKPLDGEYFTLQIRGRERFEMFRELNEALELKDAQAGKEPG |
| Q186 (SEQ ID NO: 369) | QVQLQESGGGLVQAGGSLRLSCAASGRTFSEYAMGWFRQAPGKEREFVATI SWSGGSTYYTDSVKGRFTISRDNAKNTVYLQMNSLKPDDTAVYYCAAAGLG TVVSEWDYDYDYWGQGTQVTVSSGGGGSGGGGSASTKGPSVFPLAPSSKST SGGTAALGCLVKDYFPEPVTVSWNSGALTSGVHTFPAVLQSSGLYSLSSW TVPSSSLGTQTYICNVNHKPSNTKVDKKVEPKSCDKTHTCPPCPAPELLGG KKKPLDGEYFTLQIRGRERFEMFRELNEALELKDAQAGKEPG |

TABLE 23 Continued

Amino acid sequence of single variable domain binding sites QB-1 to 71, FE & BG; and Quad polypeptide monimers in different formats, all containing single variable domain binding sites (QB-3, 10, FE or BG)

| | |
|---|---|
| QB-1 (SEQ ID NO: 370) | QVQLVESGGGLVQAGGSLRLSCAASGFPVRKANMHWYRQAPGKEREW VAAIMSKGEQTVYADSVEGRFTISRDNAKNTVYLQMNSLKPEDTAVY YCRVFVGWHYFGQGTQVTVS |
| QB-2 (SEQ ID NO: 371) | QVQLVESGGGLVQAGGSLRLSCATSGFPVYQANMHWYRQAPGKEREW VAAIQSYGDGTHYADSVKGRFTISRDNAKNTVYLQMNSLKPEDTAVY YCRAVYVGMHYFGQGTQVTVS |
| QB-0C (SEQ ID NO: 372) | QVQLVESGGGLVQAGGSLRLSCAASGFPVNYKTMWWYRQAPGKEREW VAAIWSYGHTTHYADSVKGRFTISRDNAKNTVYLQMNSLKPEDTAVY YCWWVGHNYEGQGTQVTVS |
| QB-4 (SEQ ID NO: 373) | QVQLVESGGGLVQAGGSLRLSCAASGFPVYAQNMHWYRQAPGKEREW VAAIYSHGYWTLYADSVKGRFTISRDNAKNTVYLQMNSLKPEDTAVY YCEVQVGAWYTGQGTQVTVS |
| QB-5 (SEQ ID NO: 374) | QVQLVESGGGLVQAGGSLRLSCAASGFPVFSGHMHWYRQAPGKEREW VAAILSNGDSTHYADSVKGRFTISRDNAKNTVYLQMNSLKPEDTAVY YCRVHVGAHYFGQGTQVTVS |
| QB-6 (SEQ ID NO: 375) | QVQLVESGGGLVQAGGSLRLSCAASGFPVEQGRMYWYRQAPGKEREW VAAIISHGTVTVYADSVKGRFTISRDNAKNTVYLQMNSLKPEDTAVY YCYVYVGAQYWGQGTQVTVS |
| QB-7 (SEQ ID NO: 376) | QVQLVESGGGLVQAGGSLRLSCAASGFPVLFTYMHWYRQAPGKEREW VAAIWSSGNSTWYADSVKGRFTISRDNAKNTVYLQMNSLKPEDTAVY YCFVKVGNWYAGQGTQVTVS |
| QB-8 (SEQ ID NO: 377) | QVQLVESGGGLVQAGGSLRLSCAASGFPVNAGNMHWYRQAPGKEREW VAAIQSYGRTTYYADSVKGRFTISRDNAKNTVYLQMNSLKPEDTAVY YCRVFVGMHYFGQGTQVTVS |
| QB-9 (SEQ ID NO: 378) | QVQLVESGGGLVQAGGSLRLSCAASGFPVSSSTMTWYRQAPGKEREW VAAINSYGWETHYADSVKGRFTISRDNAKNTVYLQMNSLKPEDTAVY YCYVYVGGSYIGQGTQVTVS |
| QB-A0 | QVQLVESGGGLVQAGGSLRLSCAASGFPVQSHYMRWYRQAPGKEREW VAAIESTGHHTAYADSVKGRFTISRDNAKNTVYLQMNSLKPEDTAVY |
| (SEQ ID NO: 379) | YCTVYVGYEYHGQGTQVTVS |
| QB-11 (SEQ ID NO: 380) | QVQLVESGGGLVQAGGSLRLSCAASGFPVETENMHWYRQAPGKEREW VAAIYSHGMWTAYADSVKGRFTISRDNTKNTVYLQMNSLKPEDTAVY YCEVEVGKWYFGQGTQVTVS |
| QB-12 (SEQ ID NO: 381) | QVQLVESGGGLVQAGGSLRLSCAASGFPVKASRMYWYRQAPGKEREW VAAIQSFGEVTWYADSVKGRFTISRDNAKNTVYLQMNSLKPEDTAVY YCYVWVGQEYWGQGTQVTVS |
| QB-13 (SEQ ID NO: 382) | QVQLVESGGGLVQAGGSLRLSCAASGFPVYASNMHWYRQAPGKEREW VAAIESQGYMTAYADSVKGRFTISRDNAKNTVYLQMNSLKPEDTAVY YCWVIVGEYYVGQGTQVTVS |
| QB-14 (SEQ ID NO: 383) | QVQLVESGGGLVQAGGSLRLSCAASGFPVQAREMEWYRQAPGKEREW VAAIKSTGTYTAYAYSVKGRFTISRDNAKNTVYLQMNSLKPEDTAVY YCYVYVGSSYIGQGTQVTVS |
| QB-15 (SEQ ID NO: 384) | QVQLVESGGGLVQAGGSLRLSCAASGFPVKNFEMEWYRKAPGKEREW VAAIQSGGVETYYADSVKGRFTISRDNAKNTVYLQMNSLKPEDTAVY YCFVYVGRSYIGQGTQVTVS |
| QB-16 (SEQ ID NO: 385) | QVQLVESGGGLVQAGGSLRLSCAASGFPVAYKTMWWYRQAPGKEREW VAAIESYGIKWTRYADSVKGRFTISRDNAKNTVYLQMNSLKPEDTAV YYCIVWVGAQYHGQGTQVTVS |
| QB-17 (SEQ ID NO: 386) | QVQLVESGGGLVQAGGSLRLSCAASGFPVAGRNMWWYRQAPGKEREW VAAIYSSGTYTEYADSVKGRFTISRDNAKNTVYLQMNSLKPEDTAVY YCHVWVGSLYKGQGTQVTVS |
| QB-18 (SEQ ID NO: 387) | QVQLVESGGGLVQAGGSLRLSCAASGFPVKHARMWWYRQAPGKEREW VAAIDSHGDTTWYADSVKGRFTISRDNAKNTVYLQMNSLKPEDTAVY YCYVYVGASYWGQGTQVTVS |
| QB-19 (SEQ ID NO: 388) | QVQLVESGGGLVQAGGSLRLSCAASGFPVNSHEMTWYRQAPGKEREW VAAIQSTGTVTEYADSVKGRFTISRDNAKNTVYLQMNSLKPEDTAVY YCYVYVGSSYLGQGTQVTVS |
| QB-20 (SEQ ID | QVQLVESGGGLVQAGGSLRLSCAASGFPVEQREMEWYRQAPGKEREW VAAIDSNGNYTFYADSVKGRFTISRDNAKNTVYLQMNSLKPEDTAVY |
| NO: 389) | YCYVYVGKSYIGQGTQVTVS |
| QB-21 (SEQ ID NO: 390) | QVQLVESGGGLVQAGGSLRLSCAASGFPVKHHWMFWYRQAPGKEREW VAAIKSYGYGTEYADSVKGRFTISRDNAKNTVYLQMNSLKPEDTAVY YCFVGVGTHYAGQGTQVTVS |
| QB-23 (SEQ ID NO: 391) | QVQLVESGGGLVQAGGSLRLSCAASGFPVYAAEMEWYRQAPGKEREW VAAISSQGTITYYADSVKGRFTISRDNAKNTVYLQMNSLKPEDTAVY YCFVYVGKSYIGQGTQVSVS |
| QB-25 (SEQ ID NO: 392) | QVQLVESGGGLVQAGGSLRLSCAASGFPVHAWEMAWYRQAPGKEREW VAAIRSFGSSTHYADSVKGRFTISRDNAKNTVYLQMNSLKPEDTAVY YCNVKDFGTHHYAYDYWGQGTQVTVS |
| QB-26 (SEQ ID NO: 393) | QVQLVESGGGLVQAGGSLRLSCAASGFPVNTWWMHWYRQAPGKEREW VAAITSWGFRTYYADSVKGRFTISRDNAKNTVYLQMNSLKPEDTAVY YCNVKDKGMAVQWYDYWGQGTQVTVS |
| QB-28 (SEQ ID NO: 394) | QVQLVESGGGLVQAGGSLRLSCAASGFPVYHSTMFWYRQAPGKEREW VAAIYSSGQHTYYADSVKGRFTISRDNAKNTVYLQMNSLKPEDTAVY YCNVKDSGQWRQEYDYWGQGTQVTVS |
| QB-29 (SEQ ID NO: 395) | QVQLVESGGGLVQAGGSLRLSCAASGFPVEHEMAWYRQAPGKEREWV AAIRSMGRKTIYADSVKGRFTISRDNAKNTVYIQMNSIKPEDTAVYY CNVKDFGYTWHEYDYWGQGTQVTVS |
| QB-30 (SEQ ID NO: 396) | QVQLVESGGGLVQAGGSLRLSCAASGFPVTMAWMWWYRQAPGKEREW VAAIRSEGVRTYYADSVKGRFTISRDNAKNTVYLQMNSLKPEDTAVY YCNVKDYGQAHAYYDYWGQGTQVTVS |
| QB-31 (SEQ ID NO: 397) | QVQLVESGGGLVQAGGSLRLSCAASGFPVNSHFMEWYRQAPGKEREW VAAIQHSSGFHTYYADSVKGRFTISRDNAKNTVYLQMNSLKPEDTAV YYCNVKDTGTTEDYDYWGQGTQVTVS |
| QB-32 (SEQ ID NO: 398) | QVQLDESGGGLVQAGGSLRLSCAASGFPVYHAWMEWYRQAPGKEREW VAAITSSGRHTYYADSVKGRFTISRDNAKNTVYLQMNSLKPEDTAVY YCNVKDAGRVYNSYDYWGQGTQVTVS |
| QB-33 (SEQ ID NO: 399) | QVQLVESGGGLVQAGGSLRLSCAASGFPVAHAWMEWYRQAPGKEREW VAAITSYGYKTYYADSVKGRFTISRDNAKNTVYLQMNSLKPEDTAVY YCNVKDTGTYRFYYDYWGQGTQVTVS |
| QB-34 (SEQ ID NO: 400) | QVQLVESGGGLVQAGGSLRLSCAASGFPVWNQTMVWYRQAPGKEREW VAAIWSMGHTYYADSVKGRFTISRDNAKNTVYLQMNSLKPEDTAVYY CNVKDAGVYNRYYDYWGQGTQVTVS |
| QB-35 (SEQ ID NO: 401) | QVQLVESGGGLVQAGGSLRLSCAASGFPVEHYWMEWYRQAPGKEREW VAAITSFGYRTYYADSVKGRFTISRDNAKNTVYLQMNSLKPEDTAVY YCNVKDWGFASHAYDYWGQGIQVTVS |
| QB-36 (SEQ ID NO: 402) | QVQLVESGGGLVQAGGSLRLSCAASGFPEIAWEMAWYRQAPGKEREW VAAIRSFGERTLYADSVKGRFTISRDNAKNTVYLQMNSLKPEDTAVY YCNVKDFGWQHQEYDYWGQGTQVTVS |
| QB-37 (SEQ ID NO: 403) | QVQLVESGGGLVQAGGSLRLSCAASGFPVYHAYMEWYRQAPGKEREW VAAIYSNGEHTYYADSVKGRFTISRDNAKNTVYLQMNSLKPEDTAVY YCNVKDSGSFNQAYDYWGQGTQVTVS |
| QB-38 (SEQ ID NO: 404) | QVQLVESGGGLVQAGGSLRLSCAASGFPVEWSHMHWYRQAPGKEREW VAAIVSKGGYTLYADSVKGRFTISRDNAKNTVYLQMNSLKPEDTAVY YCNVKDYGVHFKRYDYWGQGTQVTVI |
| QB-39 (SEQ ID NO: 405) | QVQLVESGGGLVQAGGSLRLSCAASGFPVFHVWMEWYRQAPGKEREW VAAIDSAGWHTYYADSVKGRFTISRDNAKNTVYLQMNSLKPEDTAVY YCNVKDAGNTTSAYDYWGQGTQVTVS |
| QB-40 (SEQ ID NO: 406) | QVQLVESGGGLVQAGGSLRLSCAASGFPVYYNWMEWYRQAPGKEREW VAAIHSNGDETFYADSVKGRFTISRDNAKNTVYLQMNSLKPEDTAVY YCNVKDIDAEAYAYDYWGQGTQVTVS |
| QB-41 (SEQ ID NO: 407) | QVQLVESGGGLVQAGGSLRLSCAASGFPVYHVWMEWYRQAPGKEREW VAAITSSGSHTYYADSVKGRFTISRDNAKNTVYLQMNSLKPEDTAVY YCNVKDSGQWRVQYDYWGQGTQVTVS |
| QB-42 (SEQ ID NO: 408) | QVQLVESGGGLVQAGGSLRLSCAASGFPVYWHHMHWYRQAPGKEREW VAAIISWGWYTTYADSVKGRFTISRDNAKNTVYLQMNSLKPEDTAVY YCNVKDHGAQNQMYDYWGQGTQVTVS |
| QB-45 (SEQ ID NO: 409) | QVQLVESGGGLVQAGGSLRLSCAASGFPVYRDRMAWYRQAPGKEREW VAAIYSAGQQTRYADSVKGRFTISRDNAKNTVYLQMNSLKPEDTAVY YCNVKDVGHHYEYYDYWGQGTQVTVS |
| QB-46 | QVQLVESGGGLVQAGGSLRLSCAASGFPVDNGYMHWYRQAPGKEREW |
| (SEQ ID NO: 410) | VAAIDSYGWHTIYADSVKGRFTISRDNAKNTVYLQMNSLKPEDTAVY YCNVKDKGQMRAAYDYWGQGTQVTVS |
| QB-47 (SEQ ID NO: 411) | QVQLVESGGGLVQAGGSLRLSCAASGFPVSWHSMYWYRQAPGKEREW VAAIFSEGDWTYYADSVKGRFTISRDNAKNTVYLQMNSLKPEDTAVY YCNVKDYGSSYYKYDYWGQGTQVTVS |
| QB-48 (SEQ ID NO: 412) | QVQLVESGGGLVQAGGSLRLSCAASGFPVSQSVMAWYRQAPGKEREW VAAIYSKGQYTHYADSVKGRFTISRDNAKNTVYLQMNSLKPEDTAVY YCNVKDAGSSYWDYDYWGQGTQVTVS |
| QB-49 (SEQ ID NO: 413) | QVQLVESGGGSVQAGGSLRLSCAASGSIGQIEYLGWFRQAPGKEREG VAALNTWTGRTYYADSVKGRFTVSLDNAKNTVYLQMNSLKPEDTALY YCAAARWGRTKPLNTYYYSYWGQGTPVTVS |
| QB-50 (SEQ ID NO: 414) | QVQLVESGGGSVQAGGSLRLSCAASGYIDKIVYLGWFRQAPGKEREG VAALYTLSGHTYYADSVKGRFTVSLDNAKNTVYLQMNSLKPEDTALY YCAAATEGHAHALYRLHYYWGQGTQVTVS |
| QB-51 (SEQ ID NO: 415) | QVQLVESGGGLVQAGGSLRLSCAASGFPVYQGEMHWYRQAPGKEREW VAAIRSTGVQTWYADSVKGRFTISRDNAKNTVYLQMNSLKPEDTAVY YCRVWVGTHYFGQGTQVTVS |
| QB-52 (SEQ ID NO: 416) | QVQLVESGGGSVQAGGSLRLSCAASGNIQRIYYLGWFRQAPGKEREG VAALMTYTGHTYYADSVKGRFTVSLDNAKNTVYLQMNSLKPEDTALY YCAAAYVGAENPLPYSMYGYWGQGTQVTVS |
| QB-53 (SEQ ID NO: 417) | QVQLVESGGGSVQAGGSLRLSCAASGQISHIKYLGWFRQAPGKEREG VAALITRWGQTYYADSVKGRFTVSLDNAKNTVYLQMNSLKPEDTALY YCAAADYGASDPLWFIHYLYWGQGTQVTVS |
| QB-55 (SEQ ID NO: 418) | QVQLVESGGGSVQAGGSLRLSCAASGKIWTIKYLGWFRQAPGKEREG VAALMTRWGYTYYADSVKGRFTVSLDNAKNTVYLQMNSLKPEDTALY YCAAANYGSNFPLAEEDYWYWGQGTQVTVS |
| QB-56 (SEQ ID NO: 419) | QVQLVESGGGSVQAGGSLRLSCAASGNISQIHYLGWFRQAPGKEREG VAALNTDYGYTYYADSVKGRFTVSLDNAKNTVYLQMNSLKPEDTALY YCAAAYYFGDDIPLWWEAYSYWGQGTQVTVS |
| QB-58 (SEQ ID | QVQLVESGGGSVQAGGSLRLSCAASGNISTIEYLGWFRQAPGKEREG VAALYTWHGQTYYADSVKGRFTVSLDNAKNTVYLQMNSLKPEDTALY |
| NO: 420) | YCAAARWGRHMPLSATEYSYWGQGTQVTVS |
| QB-59 (SEQ ID NO: 421) | QVQLVESGGGSVQAGGSLRLSCAASGNIESIYYLGWFRQAPGKEREG VAALWTGDGETYYADSVKGRFTVSLDNAKNTVYLQMNSLKPEDTALY YCAAAAWGNSAPLTTYRYYYWGQGTQVTVS |
| QB-61 (SEQ ID NO: 422) | QVQLVESGGGSVQAGGSLRLSCAASGFIYGITYLGWFRQAPGKEREG VAALVTWNGQTYYADSVKGRFTVSLDNAKNTVYLQMNSLKPEDTALY YCAAADWGYDWPLWDEWYWYWGQGTQVTVS |
| QB-62 (SEQ ID NO: 423) | QVQLVESGGGSVQAGGSLRLSCAASGTIADIKYLGWFRQAPGKEREG VAALMTRWGSTYYADSVKGRFTVSLDNAKNTVYLQMNSLKPEDTALY YCAAANYGANYPLYSQQYSYWGQGTQVTVS |
| QB-63 (SEQ ID NO: 424) | QVQLVESGGGSVQAGGSLRLSCAASGSISSIKYLGWFRQAPGKEREG VAALMTRWGMTYYADSVKGRFTVSLDNAKNTVYLQMNSLKPEDTALY YCAAANYGANEPLQYTHYNYWGQGTQVTVS |
| QB-64 (SEQ ID NO: 425) | QVQLVESGGGSVQAGGSLRLSCAASGEIESIFYLGWFRQAPGKEREG VAALYTYVGQTYYADSVKGRFTVSLDNAKNTVYLQMNSLKPEDTALY YCAAASYGAAHPLSIMRYYYWGQGTQVTVS |
| QB-66 (SEQ ID NO: 426) | QVQLVESGGGSVQAGGSLRLSCAASGSIQAITYLGWFRQAPGKEREG VAALVTWNGQTYYADSVKGRFTVSLDNAKNTVYLQMNSLKPEDTALY YCAAADWGYDWPLWDEWYWYWGQGTQVTVS |
| QB-67 (SEQ ID NO: 427) | QVQLVESGGGSVQAGGSLRLSCAASGSISSITYLGWFRQAPGKEREG VAALVTYSGNTYYADSVKGRFTVSLDNAKNTVYLQMNSLKPEDTALY YCAAATWGHSWPLYNDEYWYWGQGSQVTVS |
| QB-68 (SEQ ID NO: 428) | QVQLVESGGGSVQAGGSLRLSCAASGSISSITYLGWFRQAPGKEREG VAALITVNGHTYYADSVKGRFTVSLDNAKNTVYLQMNSLKPEDTALY YCAAAAWGYAWPLHQDDYWYWGQGTQVTVS |
| QB-69 (SEQ ID NO: 429) | QVQLVESGGGSVQAGGSLRLSCAASGSISSITYLGWFRQAPGKEREG VAALNTFNGTTYYADSVKGRFTVSLDNAKNTVYLQMNSLKPEDTALY YCAAATWGYSWPLIAEYNWYWGQGTQVTVS |
| QB-71 (SEQ ID NO: 430) | QVQLVESGGGSVQAGGSLRLSCAASGSISSITYLGWFRQAPGKEREG VAALKTQAGFTYYADSVKGRFTVSLDNAKNTVYLQMNSLKPEDTALY YCAAANWGYSWPLYEADDWYWGQGTQVTVS |
| QB-BG (SEQ ID NO: 431) | QVQLVESGGGLVQAGGSLRLSCAASGFPVYNTWMEWYRQAPGKEREW VAAITSHGYKTYYADSVKGRFTISRDNAKNTVYLQMNSLKPEDTAVY YCNVKDEGDMFTAYDYWGQGTQVTVS |
| QB-FE (SEQ ID NO: 432) | QVQLVESGGGSVQAGGSLRLSCAASGTIAHIKYLGWFRQAPGKEREG VAALMTKWGQTYYADSVKGRFTVSLDNAKNTVYLQMNSLKPEDTALY YCAAASYGANFPLKASDYSYWGQGTQVTVS |
| QB-0C mIg-TD (SEQ ID NO: 433) (QB-3 is the sequence not in bold) | QVQLVESGGGLVQAGGSLRLSCAASGFPVNYKTMWWYRQAPGKEREW VAAIWSYGHTTHYADSVKGRFTISRDNAKNTVYLQMNSLKPEDTAVY YCWWVGHNYEGQGTQVTVSSGGGGSGGGGSEPKSCDKTHTCPPCPA PELLGGPSVFLFPPKPKDTLMISRTPEVTCVVVDVSHEDPEVKFNWY VDGVEVHNAKTKPREEQYNSTYRWSVLTVLHQDWLNGKEYKCKVSN KALPAPIEKTISKAKGQPREPQVYTLPPSRDELTKNQVSLTCLVKGF YPSDIAVEWESNGQPENNYKTTPPVLDSDGSFFLYSKLTVDKSRWQQ GNVFSCSVMHEALHNHYTQKSLSLSPGKKKKPLDGEYFTLQIRGRER FEMFRELNEALELKDAQAGKEPG |
| QB-A0 mIg-TD (SEQ ID NO: 434) (QB-10 is the sequence not in bold) | QVQLVESGGGLVQAGGSLRLSCAASGFPVQSHYMRWYRQAPGKEREW VAAIESTGHHTAYADSVKGRFTISRDNAKNTVYLQMNSLKPEDTAVY YCTVYVGYEYHGQGTQVTVSSGGGGSGGGGSEPKSCDKTHTCPPCPA PELLGGPSVFLFPPKPKDTLMISRTPEVTCVVVDVSHEDPEVKFNWY VDGVEVHNAKTKPREEQYNSTYRWSVLTVLHQDWLNGKEYKCKVSN KALPAPIEKTISKAKGQPREPQVYTLPPSRDELTKNQVSLTCLVKGF YPSDIAVEWESNGQPENNYKTTPPVLDSDGSFFLYSKLTVDKSRWQQ GNVFSCSVMHEALHNHYTQKSLSLSPGKKKKPLDGEYFTLQIRGRER FEMFRELNEALELKDAQAGKEPG |
| QB-BG mIg-TD (SEQ ID NO: 435) QB-G B is the sequ- | QVQLVESGGGLVQAGGSLRLSCAASGFPVYNTWMEWYRQAPGKEREW VAAITSHGYKTYYADSVKGRFTISRDNAKNTVYLQMNSLKPEDTAVY YCNVKDEGDMFTAYDYWGQGTQVTVSSGGGGSGGGGSEPKSCDKTHT CPPCPAPELLGGPSVFLFPPKPKDTLMISRTPEVTCVVVDVSHEDPE VKFNWYVDGVEVHNAKTKPREEQYNSTYRVVSVLTVLHQDWLNGKEY KCKVSNKALPAPIEKTISKAKGQPREPQVYTLPPSRDELTKNQVSLT CLVKGFYPSDIAVEWESNGQPENNYKTTPPVLDSDGSFFLYSKLTVD KSRWQQGNVFSCSVMHEALHNHYTQKSLSLSPGKKKKPLDGEYFTLQ |
| ence not in bold) | IRGRERFEMFRELNEALELKDAQAGKEPG |
| QB-FE mIg-TD (SEQ ID NO: 436) (QB-FE is the sequenc e not in bold) | QVQLVESGGGSVQAGGSLRLSCAASGTIAHIKYLGWFRQAPGKEREG VAALMTKWGQTYYADSVKGRFTVSLDNAKNTVYLQMNSLKPEDTALY YCAAASYGANFPLKASDYSYWGQGTQVTVSSGGGGSGGGGSEPKSCD KTHTCPPCPAPELLGGPSVFLFPPKPKDTLMISRTPEVTCVVVDVSH EDPEVKFNWYVDGVEVHNAKTKPREEQYNSTYRVVSVLTVLHQDWLN GKEYKCKVSNKALPAPIEKTISKAKGQPREPQVYTLPPSRDELTKNQ VSLTCLVKGFYPSDIAVEWESNGQPENNYKTTPPVLDSDGSFFLYSK LTVDKSRWQQGNVFSCSVMHEALHNHYTQKSLSLSPGKKKKPLDGEY FTLQIRGRERFEMFRELNEALELKDAQAGKEPG |

Table 24. Amino acid sequences of ACE2 in different formats for capturing or neutralizing SARS-CoV or SARS-CoV-2 or other related coronavirus strains. Each of the sequences outlined in this table can be further generated with or without a N- or C-terminus affinity tag as outlined in Table 22 and shown schematically in FIGS. 5A-C. ACE2 extracellular domain can be composed of either amino acids 18-615 or amino acids 18-740. The tetramerization domain (TD) is preferably TD from p53 but not limited by it.

The ACE2 sequences for use in the present invention are underlined and are intended to be read as written (ie, as part of the full sequence including non-underlined part) or alternatively without the futher underlined sequence.

In an embodiment, the polypeptide of the invention comprises a sequence that is shown in bold in this Table 24. In an embodiment, the polypeptide of the invention comprises, in N- to C-terminal direction, a binding domain or peptide; and a sequence that is shown in bold in this Table 24. The binding domain may be any binding domain disclosed herein, eg, a VH, VL, VHH, dAb or scFv.

The present disclosure further comprises each underlined sequence without the following bold sequence, eg, to provide a binding site or domain for use in any polypeptide or mutimer of the invention herein. The present disclosure further comprises each bold sequence without the immediately preceding underlined sequence, eg, to for use in any polypeptide or mutimer of the invention herein (such as as part of a polypeptide comprising a binding domain or peptide immediately preceding the bold sequence).

| Name | Sequence |
|---|---|
| ACE2(18-615) (SEQ ID NO: 437) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYAD |
| ACE2(18-615)-TD (SEQ ID NO: 438) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYADKKKPLDGEYFTLQIRGRERFEMFRELNEALELKDAQAGK EPG |
| ACE2(18-615) monomeric Ig-TD (SEQ ID NO: 439) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS |
| | NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYAD EPKSCDKTHTAPELLGGPSVFLFPPKPKDTLMISRTPEV TCVWDVSHEDPEVKFNWYVDGVEVHNAKTKPREEQYNSTYRWSVLTV LHQDWLNGKEYKCKVSNKALPAPIEKTISKAKGQPREPQVYTLPPSRDE LTKNQVSLTCLVKGFYPSDIAVEWESNGQPENNYKTTPPVLDSDGSFFL YSKLTVDKSRWQQGNVFSCSVMHEALHNHYTQKSLSLSPGKKKKPWGE YFTLQIRGRERFEMFRELNEALELKDAQAGKEPG |
| ACE2(18-615) Ig-TD (SEQ ID NO: 440) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYAD EPKSCDKTHTAPELLGGPSVFLFPPKPKDTLMISRTPEV TCVWDVSHEDPEVKFNWYVDGVEVHNAKTKPREEQYNSTYRWSVLTV LHQDWLNGKEYKCKVSNKALPAPIEKTISKAKGQPREPQVYTLPPSRDE LTKNQVSLTCLVKGFYPSDIAVEWESNGQPENNYKTTPPVLDSDGSFFL YSKLTVDKSRWQQGNVFSCSVMHEALHNHYTQKSLSLSPGKASTKGPSV FPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKT HTCPPCPAPELLGGPSVFLFPPKPKDTLMISRTPEVTCVWDVSHEDPE VKFNWYVDGVEVHNAKTKPREEQYNSTYRWSVLTVLHQDWLNGKEYKC KVSNKALPAPIEKTISKAKGQPREPQVYTLPPSRDELTKNQVSLTCLVK GFYPSDIAVEWESNGQPENNYKTTPPVLDSDGSFFLYSKLTVDKSRWQQ GNVFSCSVMHEALHNHYTQKSLSLSPGKKKKPLDGEYFTLQIRGRERFE MFRELNEALELKDAQAGKEPG |
| ACE2(18-740) (SEQ ID NO: 441) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN |
| | MLRLGKSEPWTLALENVVGAKNMNVRPLLNYFEPLFTWLKDQNKNSFVG WSTDWSPYADQSIKVRISLKSALGDKAYEWNDNEMYLFRSSVAYAMRQY FLKVKNQMILFGEEDVRVANLKPRISFNFFVTAPKNVSDIIPRTEVEKA IRMSRSRINDAFRLNDNSLEFLGIQPTLGPPNQPPVS |
| ACE2(18-740)-TD (SEQ ID NO: 442) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYADQSIKVRISLKSALGDKAYEWNDNEMYLFRSSVAYAMRQY FLKVKNQMILFGEEDVRVANLKPRISFNFFVTAPKNVSDIIPRTEVEKA IRMSRSRINDAFRLNDNSLEFLGIQPTLGPPNQPPVSKKKPLDGEYFTL QIRGRERFEMFRELNEALELKDAQAGKEPG |
| ACE2(18-740) monomeric Ig-TD (SEQ ID NO: 443) | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYADQSIKVRISLKSALGDKAYEWNDNEMYLFRSSVAYAMRQY FLKVKNQMILFGEEDVRVANLKPRISFNFFVTAPKNVSDIIPRTEVEKA IRMSRSRINDAFRLNDNSLEFLGIQPTLGPPNQPPVS EPKSCDKTHTAP ELLGGPSVFLFPPKPKDTLMISRTPEVTCVWDVSHEDPEVKFNWYVDG VEVHNAKTKPREEQYNSTYRVVSVLTVLHQDWLNGKEYKCKVSNKALPA PIEKTISKAKGQPREPQVYTLPPSRDELTKNQVSLTCLVKGFYPSDIAV EWESNGQPENNYKTTPPVLDSDGSFFLYSKLTVDKSRWQQGNVFSCSVM HEALHNHYTQKSLSLSPGKKKKPLDGEYFTLQIRGRERFEMFRELNEAL ELKDAQAGKEPG |
| ACE2(18-740) Ig-TD (SEQ ID NO: | QSTIEEQAKTFLDKFNHEAEDLFYQSSLASWNYNTNITEENVQNMNNAG DKWSAFLKEQSTLAQMYPLQEIQNLTVKLQLQALQQNGSSVLSEDKSKR LNTILNTMSTIYSTGKVCNPDNPQECLLLEPGLNEIMANSLDYNERLWA WESWRSEVGKQLRPLYEEYVVLKNEMARANHYEDYGDYWRGDYEVNGVD |
| 444) | GYDYSRGQLIEDVEHTFEEIKPLYEHLHAYVRAKLMNAYPSYISPIGCL PAHLLGDMWGRFWTNLYSLTVPFGQKPNIDVTDAMVDQAWDAQRIFKEA EKFFVSVGLPNMTQGFWENSMLTDPGNVQKAVCHPTAWDLGKGDFRILM CTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEK WRWMVFKGEIPKDQWMKKWWEMKREIVGVVEPVPHDETYCDPASLFHVS NDYSFIRYYTRTLYQFQFQEALCQAAKHEGPLHKCDISNSTEAGQKLFN MLRLGKSEPWTLALENVVGAKNMNVRPLLNYFEPLFTWLKDQNKNSFVG WSTDWSPYADQSIKVRISLKSALGDKAYEWNDNEMYLFRSSVAYAMRQY FLKVKNQMILFGEEDVRVANLKPRISFNFFVTAPKNVSDIIPRTEVEKA IRMSRSRINDAFRLNDNSLEFLGIQPTLGPPNQPPVS ASTKGPSVFPLA PSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCP PCPAPELLGGPSVFLFPPKPKDTLMISRTPEVTCVWDVSHEDPEVKFN WYVDGVEVHNAKTKPREEQYNSTYRVVSVLTVLHQDWLNGKEYKCKVSN KALPAPIEKTISKAKGQPREPQVYTLPPSRDELTKNQVSLTCLVKGFYP SDIAVEWESNGQPENNYKTTPPVLDSDGSFFLYSKLTVDKSRWQQGNVF SCSVMHEALHNHYTQKSLSLSPGKKKKPLDGEYFTLQIRGRERFEMFRE LNEALELKDAQAGKEPG |

Table 25. Amino acid sequences of immunoglobulin (Ig) binding domains. Sequences are shown without affinity tag but any of the affinity tags outlined in Table 22 can be included at the N- or C-terminus of the Ig binding domain. Each of the Ig binding domains can be linked together without flexible linker as shown in this table or optionally be linked together via a flexible linker. Examples of flexible linker sequences are shown in Table 26. C1-C3 domains are Ig binding domains from Protein G. EDABC domains are Ig binding domains from Protein A. B1-B5 domains are Ig binding domains from Protein L. Each Ig binding domain combination shown in this table can be formatted into different Quad formats by linking them to sequences shown in Table 27 or as outlined schematically in FIGS. 6A-D.

Each binding domain in this table may be used as a binding domain comprised by a polypeptide or multimer of the invention.

| SEQ ID | Ig binding domains | Sequence |
|---|---|---|
| 445 | C1-C3 | TYKLILNGKTLKGETTTEAVDAATAEKVFKQYANDNGVDG EWTYDDATKTFTVTETYKLVINGKTLKGETTTEAVDAATA EKVFKQYANDNGVDGEWTYDDATKTFTVTETYKLVINGKT LKGETTTKAVDAETAEKAFKQYANDNGVDGVWTYDDATKT FTVTE |
| 446 | C1 | TYKLILNGKTLKGETTTEAVDAATAEKVFKQYANDNGVDG EWTYDDATKTFTVTE |
| 447 | (C1)₂ | TYKLILNGKTLKGETTTEAVDAATAEKVFKQYANDNGVDG EWTYDDATKTFTVTETYKLILNGKTLKGETTTEAVDAATA EKVFKQYANDNGVDGEWTYDDATKTFTVTE |
| 448 | (C1)₃ | TYKLILNGKTLKGETTTEAVDAATAEKVFKQYANDNGVDG EWTYDDATKTFTVTETYKLILNGKTLKGETTTEAVDAATA EKVFKQYANDNGVDGEWTYDDATKTFTVTETYKLILNGKT LKGETTTEAVDAATAEKVFKQYANDNGVDGEWTYDDATKT FTVTE |
| 449 | C2 | TYKLVINGKTLKGETTTEAVDAATAEKVFKQYANDNGVDG EWTYDDATKTFTVTE |
| 450 | (C2)₂ | TYKLVINGKTLKGETTTEAVDAATAEKVFKQYANDNGVDG EWTYDDATKTFTVTETYKLVINGKTLKGETTTEAVDAATA EKVFKQYANDNGVDGEWTYDDATKTFTVTE |
| 451 | (C2)₃ | TYKLVINGKTLKGETTTEAVDAATAEKVFKQYANDNGVDG EWTYDDATKTFTVTETYKLVINGKTLKGETTTEAVDAATA EKVFKQYANDNGVDGEWTYDDATKTFTVTETYKLVINGKT LKGETTTEAVDAATAEKVFKQYANDNGVDGEWTYDDATKT FTVTE |
| 452 | EDABC | AQHDEAQQNAFYQVLNMPNLNADQRNGFIQSLKDDPSQSA NVLGEAQKLNDSQAPKADAQQNKFNKDQQSAFYEILNMPN LNEEQRNGFIQSLKDDPSQSTNVLGEAKKLNESQAPKADN NFNKEQQNAFYEILNMPNLNEEQRNGFIQSLKDDPSQSAN LLAEAKKLNESQAPKADNKFNKEQQNAFYEILHLPNLNEE QRNGFIQSLKDDPSQSANLLAEAKKLNDAQAPKADNKFNK EQQNAFYEILHLPNLTEEQRNGFIQSLKDDPSVSKEILAE AKKLNDAQAPK |
| 453 | D | ADAQQNKFNKDQQSAFYEILNMPNLNEEQRNGFIQSLKDD PSQSTNVLGEAKKLNESQAPK |
| 454 | (D)₂ | ADAQQNKFNKDQQSAFYEILNMPNLNEEQRNGFIQSLKDD PSQSTNVLGEAKKLNESQAPKADAQQNKFNKDQQSAFYEI LNMPNLNEEQRNGFIQSLKDDPSQSTNVLGEAKKLNESQA PK |
| 455 | (D)₃ | ADAQQNKFNKDQQSAFYEILNMPNLNEEQRNGFIQSLKDD PSQSTNVLGEAKKLNESQAPKADAQQNKFNKDQQSAFYEI LNMPNLNEEQRNGFIQSLKDDPSQSTNVLGEAKKLNESQA PKADAQQNKFNKDQQSAFYEILNMPNLNEEQRNGFIQSLK DDPSQSTNVLGEAKKLNESQAPKADAQQNKFNKDQQSAFY EILNMPNLNEEQRNGFIQSLKDDPSQSTNVLGEAKKLNES QAPK |
| 456 | B1-B5 | KEETPETPETDSEEEVTIKANLIFANGSTQTAEFKGTFEK ATSEAYAYADTLKKDNGEYTVDVADKGYTLNIKFAGKEKT PEEPKEEVTIKANLIYADGKTQTAEFKGTFEEATAEAYRY ADALKKDNGEYTVDVADKGYTLNIKFAGKEKTPEEPKEEV TIKANLIYADGKTQTAEFKGTFEEATAEAYRYADLLAKEN GKYTVDVADKGYTLNIKFAGKEKTPEEPKEEVTIKANLIY ADGKTQTAEFKGTFAEATAEAYRYADLLAKENGKYTADLE DGGYTINIRFAGKKVDEKPEEKEQVTIKENIYFEDGTVQT ATFKGTFAEATAEAYRYADLLSKEHGKYTADLEDGGYTIN IRFAG |
| 457 | B4 | KEKTPEEPKEEVTIKANLIYADGKTQTAEFKGTFAEATAE AYRYADLLAKENGKYTADLEDGGYTINIRFAG |
| 458 | (B4)₂ | KEKTPEEPKEEVTIKANLIYADGKTQTAEFKGTFAEATAE AYRYADLLAKENGKYTADLEDGGYTINIRFAGKEKTPEEP KEEVTIKANLIYADGKTQTAEFKGTFAEATAEAYRYADLL AKENGKYTADLEDGGYTINIRFAG |
| 459 | (B4)₃ | KEKTPEEPKEEVTIKANLIYADGKTQTAEFKGTFAEATAE AYRYADLLAKENGKYTADLEDGGYTINIRFAGKEKTPEEP KEEVTIKANLIYADGKTQTAEFKGTFAEATAEAYRYADLL AKENGKYTADLEDGGYTINIRFAGKEKTPEEPKEEVTIKA NLIYADGKTQTAEFKGTFAEATAEAYRYADLLAKENGKYT ADLEDGGYTINIRFAG |
| 460 | C1-D | TYKLILNGKTLKGETTTEAVDAATAEKVFKQYANDNGVDG EWTYDDATKTFTVTEADAQQNKFNKDQQSAFYEILNMPNL NEEQRNGFIQSLKDDPSQSTNVLGEAKKLNESQAPK |
| 461 | C1-B4 | TYKLILNGKTLKGETTTEAVDAATAEKVFKQYANDNGVDG EWTYDDATKTFTVTEKEKTPEEPKEEVTIKANLIYADGKT QTAEFKGTFAEATAEAYRYADLLAKENGKYTADLEDGGYT INIRFAG |
| 462 | C2-D | TYKLVINGKTLKGETTTEAVDAATAEKVFKQYANDNGVDG EWTYDDATKTFTVTEADAQQNKFNKDQQSAFYEILNMPNL NEEQRNGFIQSLKDDPSQSTNVLGEAKKLNESQAPK |
| 463 | C2-B4 | TYKLVINGKTLKGETTTEAVDAATAEKVFKQYANDNGVDG EWTYDDATKTFTVTEKEKTPEEPKEEVTIKANLIYADGKT QTAEFKGTFAEATAEAYRYADLLAKENGKYTADLEDGGYT INIRFAG |
| 464 | B4-D | KEKTPEEPKEEVTIKANLIYADGKTQTAEFKGTFAEATAE AYRYADLLAKENGKYTADLEDGGYTINIRFAGADAQQNKF |

TABLE 26

Examples of flexible peptide linkers

| SEQ ID | Linker | Sequence |
|---|---|---|
| 469 | D1 | KPEVIDASELTPAVT |
| 470 | DDAKK | DDAKK |
| 471 | (DDAKK)₂ | DDAKKDDAKK |
| 472 | (DDAKK)₃ | DDAKKDDAKKDDAKK |
| 473 | (DDAKK)₄ | DDAKKDDAKKDDAKKDDAKK |
| 474 | (DDAKK)₅ | DDAKKDDAKKDDAKKDDAKKDDAKK |
| 475 | (DDAKK)₆ | DDAKKDDAKKDDAKKDDAKKDDAKKDDAKK |
| 476 | GS | GS |
| 477 | GGGS | GGGS |
| 478 | SGGGGS | SGGGGS |
| 479 | GGSGGGGS | GGSGGGGS |
| 480 | GG(G4S)₂ | GGGGGGSGGGGS |
| 481 | G4S | GGGGS |
| 482 | (G4S)₂ | GGGGSGGGGS |
| 483 | (G4S)₃ | GGGGSGGGGSGGGGS |
| 484 | VPGV | VPGV |
| 485 | (VPGV)G | VPGVG |
| 486 | G(VPGV)G | GVPGVG |
| 487 | GVG(VPGV)G | GVGVPGVG |
| 488 | (VPGV)G(VPGV)G | VPGVGVPGVG |

TABLE 27

Amino acid sequences of different antibody and Quad backbone including tetramerization domain (TD). Any of the Ig binding domain sequence listed in Table 25 can be linked to any of these backbone to generate multimeric Ig capture proteins with multiple Ig binding domains. Inclusion of optional flexible linker/s are not included in the sequences in this table but can be included in additional iterations as shown schematically in FIGS. 6A-D

| SEQ ID | Name | Sequence |
|---|---|---|
| 489 | Heavy chain only Fc | EPKSCDKTHTCPPCPAPELLGGPSVFLFPPKPKDTLMISR TPEVTCVVVDVSHEDPEVKFNWYVDGVEVHNAKTKPREEQ YNSTYRVVSVLTVLHQDWLNGKEYKCKVSNKALPAPIEKT ISKAKGQPREPQVYTLPPSRDELTKNQVSLTCLVKGFYPS DIAVEWESNGQPENNYKTTPPVLDSDGSFFLYSKLTVDKS RWQQGNVFSCSVMHEALHNHYTQKSLSLSPGK |
| 490 | Heavy chain only Fc-TD | EPKSCDKTHTCPPCPAPELLGGPSVFLFPPKPKDTLMISR TPEVTCVVVDVSHEDPEVKFNWYVDGVEVHNAKTKPREEQ YNSTYRVVSVLTVLHQDWLNGKEYKCKVSNKALPAPIEKT ISKAKGQPREPQVYTLPPSRDELTKNQVSLTCLVKGFYPS DIAVEWESNGQPENNYKTTPPVLDSDGSFFLYSKLTVDKS RWQQGNVFSCSVMHEALHNHYTQKSLSLSPGKKKKPLDGE YFTLQIRGRERFEMFRELNEALELKDAQAGKEPG |
| 491 | IgG | EPKSCDKTHTCPPCPAPELLGGPSVFLFPPKPKDTLMISR TPEVTCVVVDVSHEDPEVKFNWYVDGVEVHNAKTKPREEQ YNSTYRVVSVLTVLHQDWLNGKEYKCKVSNKALPAPIEKT ISKAKGQPREPQVYTLPPSRDELTKNQVSLTCLVKGFYPS DIAVEWESNGQPENNYKTTPPVLDSDGSFFLYSKLTVDKS RWQQGNVFSCSVMHEALHNHYTQKSLSLSPGK |
| 492 | IgG1-TD | ASTKGPSVFPLAPSSKSTSGGTAALGCLVKDYFPEPVTVS WNSGALTSGVHTFPAVLQSSGLYSLSSVVTVPSSSLGTQT YICNVNHKPSNTKVDKKVEPKSCDKTHTCPPCPAPELLGG PSVFLFPPKPKDTLMISRTPEVTCVVVDVSHEDPEVKFNW YVDGVEVHNAKTKPREEQYNSTYRVVSVLTVLHQDWLNGK EYKCKVSNKALPAPIEKTISKAKGQPREPQVYTLPPSRDE LTKNQVSLTCLVKGFYPSDIAVEWESNGQPENNYKTTPPV LDSDGSFFLYSKLTVDKSRWQQGNVFSCSVMHEALHNHYT QKSLSLSPGKKKKPLDGEYFTLQIRGRERFEMFRELNEAL ELKDAQAGKEPG |
| 493 | Kappa constant | RTVAAPSVFIFPPSDEQLKSGTASVVCLLNNFYPREAKVQ WKVDNALQSGNSQESVTEQDSKDSTYSLSSTLTLSKADYE KHKVYACEVTHQGLSSPVTKSFNRGEC |
| 494 | Lambda | GQPKAAPSVTLFPPSSEELQANKATLVCLISDFYPGAVTV |
| | constant | AWKADSSPVKAGVETTTPSKQSNNKYAASSYLSLTPEQWK SHRSYSCQVTHEGSTVEKTVAPTECS |
| 495 | Fab-like-TD | ASTKGPSVFPLAPSSKSTSGGTAALGCLVKDYFPEPVTVS WNSGALTSGVHTFPAVLQSSGLYSLSSVVTVPSSSLGTQT YICNVNHKPSNTKVDKKVEPKSCDKTHTKKKPLDGEYFTL QIRGRERFEMFRELNEALELKDAQAGKEPG |
| 496 | Fab-like monomeric Ig-TD | ASTKGPSVFPLAPSSKSTSGGTAALGCLVKDYFPEPVTVS WNSGALTSGVHTFPAVLQSSGLYSLSSVVTVPSSSLGTQT YICNVNHKPSNTKVDKKVEPKSCDKTHTAPELLGGPSVFL FPPKPKDTLMISRTPEVTCVVVDVSHEDPEVKFNWYVDGV EVHNAKTKPREEQYNSTYRVVSVLTVLHQDWLNGKEYKCK VSNKALPAPIEKTISKAKGQPREPQVYTLPPSRDELTKNQ VSLTCLVKGFYPSDIAVEWESNGQPENNYKTTPPVLDSDG SFFLYSKLTVDKSRWQQGNVFSCSVMHEALHNHYTQKSLS LSPGKKKKPLDGEYFTLQIRGRERFEMFRELNEALELKDA QAGKEPG |
| 497 | Dimeric-TD | EPKSCDKTHTCPPCPAPELLGGKKKPLDGEYFTLQIRGRE RFEMFRELNEALELKDAQAGKEPG |
| 498 | Fab′like-TD | ASTKGPSVFPLAPSSKSTSGGTAALGCLVKDYFPEPVTVS WNSGALTSGVHTFPAVLQSSGLYSLSSVVTVPSSSLGTQT YICNVNHKPSNTKVDKKVEPKSCDKTHTCPPCPAPELLGG KKKPLDGEYFTLQIRGRERFEMFRELNEALELKDAQAGKE PG |
| 499 | monomeric Ig-TD | EPKSCDKTHTAPELLGGPSVFLFPPKPKDTLMISRTPEVT CVVVDVSHEDPEVKFNWYVDGVEVHNAKTKPREEQYNSTY RVVSVLTVLHQDWLNGKEYKCKVSNKALPAPIEKTISKAK GQPREPQVYTLPPSRDELTKNQVSLTCLVKGFYPSDIAVE WESNGQPENNYKTTPPVLDSDGSFFLYSKLTVDKSRWQQG NVFSCSVMHEALHNHYTQKSLSLSPGKKKKPLDGEYFTLQ IRGRERFEMFRELNEALELKDAQAGKEPG |

TABLE 28

Summary of IC₅₀ from In vitro neutralization assays

| SARS-CoV-2 Inhibitor | IC₅₀ (nM) | IC₅₀ (pM) |
|---|---|---|
| Q146/Q147 | 0.39 | 390.3 |
| Q146C/Q147 | 0.18 | 175.8 |
| Q158new | 0.45 | 449.4 |

TABLE 29

Summary of IC₅₀ from In vitro neutralization assay

| SARS-CoV-2 Inhibitor | IC₅₀ (nM) | IC₅₀ (pM) | % Inhibition nCoV RBD:huACE2 Fc |
|---|---|---|---|
| Q176 | Poor neutralizer | | 26.0 |
| Q177 | 0.0649 | 64.9 | 99.9 |
| Q178 | 0.1853 | 185.3 | 96.7 |
| Q179 | 0.0328 | 32.8 | 99.9 |
| Q180/Q182 | 0.0778 | 77.8 | 99.9 |
| Q181/Q182 | 0.0154 | 15.4 | 99.9 |
| Q183/Q182 | 0.0312 | 31.2 | 99.9 |
| Q184/Q182 | 0.0342 | 34.2 | 99.9 |
| Q185 | 0.0237 | 23.7 | 99.9 |
| Q186/Q182 | 0.0148 | 14.8 | 99.9 |
| BG mlg-TD | 0.0887 | 88.7 | 99.9 |
| FE mlg-TD | 0.0800 | 80.0 | 69.1 |

TABLE 30

Comparison of IC₅₀ values of Quads with lead SARS CoV-2 neutralizing antibodies reported by Hansen et al., 2020

| Antibody | ELISA Blocking | % Inhibition nCoV RBD:huACE2 Fc |
|---|---|---|
| | IC₅₀ (pM) Blocking Cov-2 RBD | |
| Q185 | 14.7 | 98.7 |
| Q186/Q182 | 8.3 | 97.8 |
| REGN10989 | 43.3 | 98 |
| REGN10987 | 231 | 95 |
| REGN10933 | 69.3 | 99 |
| REGN10934 | 63.6 | 96 |

TABLE 31

Figure/Quad Correlation Table

| Quads | Description | Figure No. |
|---|---|---|
| Q146/Q147 | CR3022-TD | 11-A |
| Q146A/Q147 | CR3022 mlg-TD | 11-B |
| Q146C/Q147 | CR3022 Ig-TD | 11-C |
| Q158new | ACE2 Ig-TD | 11-D |
| Q176 | VHH QB-GB | 12-A |
| Q177 | VHH QB-GB-TD | |
| Q178 | VHH QB-GB IgG | 12-B |
| Q179 | VHH QB-GB Ig-TD | 12-C |
| Q180/Q182 | VHH QB-GB Fab-like Ig | 12-D |
| Q181/Q182 | VHH QB-GB Fab-like Ig-TD | 12-E |
| Q183/Q183 | VHH QB-GB Fab-like TD | 12-F |
| Q184/Q182 | VHH QB-GB Fab-like mlg-TD | 12-G |
| Q185 | VHH QB-GB dimeric TD | 12-H |
| Q186/Q182 | VHH QB-GB Fab′-like TD | 12-I |
| QB-BG VHH | VHH | 12-0 |
| QB-BG mlg-TD | VHH mlg-TD | |
| QB-FE VHH | VHH | 12-P |
| QB-FE mlg-TD | VHH mlg-TD | |

[Q182 is the chain containing a CL]

Table 32: Amino acid sequences of further engineered tandem VHH multivalent Quads and Quad formats of clinical stage mAbs targeting SARS-CoV-2.

The sequences of binding domains (eg, VH and VL) for use in the present invention are underlined and are intended to be read as written (ie, as part of the full sequence including non-underlined part) or alternatively without the futher underlined sequence.

In an embodiment, the polypeptide of the invention comprises a sequence that is shown in bold in this Table 32. In an embodiment, the polypeptide of the invention comprises, in N- to C-terminal direction, a binding domain or peptide; and a sequence that is shown in bold in this Table 32. The binding domain may be any binding domain disclosed herein, eg, a VH, VL, VHH, dAb or scFv.

The present disclosure further comprises each underlined sequence without the following bold sequence, eg, to provide a binding site or domain for use in any polypeptide or mutimer of the invention herein. The present disclosure further comprises each bold sequence without the immediately preceding underlined sequence, eg, to for use in any polypeptide or mutimer of the invention herein (such as as part of a polypeptide comprising a binding domain or peptide immediately preceding the bold sequence).

| SEQ ID No: | Quad Name | AMINO ACID SEQUENCE |
|---|---|---|
| 232 | Q177B | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD |
| | | DTAVYYCAAAGLGTVVSEWDYDYDYWGQGTQVTVSS GGGGSGGG GSKKKPLDGEYFTLQIRGRERFEMFRELNEALELKDAQAGKEPG |
| 233 | Q177C | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCVASGSIFSINAMDWYRQAPGKQR ELVAGITSGGSTNYGDFVKGRFTISRDNAKNTVYLQMDSLKPED TAVYYCAAEVGGWGPPRPDYWGHGTQVTVSSGGGGSGGGGSKKK PLDGEYFTLQIRGRERFEMFRELNEALELKDAQAGKEPG |
| 234 | Q179B | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSEPKSCDKTHTCPPCPAPELLGGPSVFLFPPKPKDTLMISRTP EVTCVWDVSHEDPEVKFNWYVDGVEVHNAKTKPREEQYNSTYR WSVLTVLHQDWLNGKEYKCKVSNKALPAPIEKTISKAKGQPRE PQVYTLPPSRDELTKNQVSLTCLVKGFYPSDIAVEWESNGQPEN NYKTTPPVLDSDGSFFLYSKLTVDKSRWQQGNVFSCSVMHEALH NHYTQKSLSLSPGKKKKPLDGEYFTLQIRGRERFEMFRELNEAL ELKDAQAGKEPG |
| 235 | Q179C | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCVASGSIFSINAMDWYRQAPGKQR ELVAGITSGGSTNYGDFVKGRFTISRDNAKNTVYLQMDSLKPED TAVYYCAAEVGGWGPPRPDYWGHGTQVTVSSGGGGSGGGGSEPK SCDKTHTCPPCPAPELLGGPSVFLFPPKPKDTLMISRTPEVTCV VVDVSHEDPEVKFNWYVDGVEVHNAKTKPREEQYNSTYRVVSVL TVLHQDWLNGKEYKCKVSNKALPAPIEKTISKAKGQPREPQVYT LPPSRDELTKNQVSLTCLVKGFYPSDIAVEWESNGQPENNYKTT PPVLDSDGSFFLYSKLTVDKSRWQQGNVFSCSVMHEALHNHYTQ KSLSLSPGKKKKPLDGEYFTLQIRGRERFEMFRELNEALELKDA |
| | | QAGKEPG |
| 236 | Q185B | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSEPKSCDKTHTCPPCPAPELLGGKKKPLDGEYFTLQIRGRERF EMFRELNEALELKDAQAGKEPG |
| 237 | Q185C | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCVASGSIFSINAMDWYRQAPGKQR ELVAGITSGGSTNYGDFVKGRFTISRDNAKNTVYLQMDSLKPED TAVYYCAAEVGGWGPPRPDYWGHGTQVTVSSGGGGSGGGGSEPK SCDKTHTCPPCPAPELLGGKKKPLDGEYFTLQIRGRERFEMFRE LNEALELKDAQAGKEPG |
| 238 | Q203 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSASTKGPSVFPLAPSSKSTSGGTAALGCLVKDYFPEPVTVSWN SGALTSGVHTFPAVLQSSGLYSLSSVVTVPSSSLGTQTYICNVN HKPSNTKVDKKVEPKSCDKTHTKKKPLDGEYFTLQIRGRERFEM FRELNEALELKDAQAGKEPG |
| 239 | Q204 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCVASGSIFSINAMDWYRQAPGKQR ELVAGITSGGSTNYGDFVKGRFTISRDNAKNTVYLQMDSLKPED TAVYYCAAEVGGWGPPRPDYWGHGTQVTVSSGGGGSGGGGSAST KGPSVFPLAPSSKSTSGGTAALGCLVKDYFPEPVTVSWNSGALT |
| | | SGVHTFPAVLQSSGLYSLSSVVTVPSSSLGTQTYICNVNHKPSN TKVDKKVEPKSCDKTHTKKKPLDGEYFTLQIRGRERFEMFRELN EALELKDAQAGKEPG |
| 240 | Q205 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSKRTVAAPSVFIFPPSDEQLKSGTASWCLLNNFYPREAKVQW KVDNALQSGNSQESVTEQDSKDSTYSLSSTLTLSKADYEKHKVY ACEVTHQGLSSPVTKSFNRGEC |
| 241 | Q177D | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSKKKPLDGEYFTLQIRGRERFEMFRELNEALELKDAQAGKEPG GGGGSGGGGSQVQLQESGGGLVQAGGSLRLSCAASGRTFSEYAM GWFRQAPGKEREFVATISWSGGSTYYTDSVKGRFTISRDNAKNT VYLQMNSLKPDDTAVYYCAAAGLGTVVSEWDYDYDYWGQGTQVT VSS |
| 242 | Q177E | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSKKKPLDGEYFTLQIRGRERFEMFRELNEALELKDAQAGKEPG GGGGSGGGGSQVQLQESGGGLVQAGGSLRLSCAASGRTFSEYAM GWFRQAPGKEREFVATISWSGGSTYYTDSVKGRFTISRDNAKNT VYLQMNSLKPDDTAVYYCAAAGLGTVVSEWDYDYDYWGQGTQVT VSSGGSGGSQVQLQESGGGLVQAGGSLRLSCAASGRTFSEYAMG |
| | | WFRQAPGKEREFVATISWSGGSTYYTDSVKGRFTISRDNAKNTV YLQMNSLKPDDTAVYYCAAAGLGTVVSEWDYDYDYWGQGTQVTV SS |
| 243 | Q185D | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSEPKSCDKTHTCPPCPAPELLGGKKKPLDGEYFTLQIRGRERF EMFRELNEALELKDAQAGKEPGGGGGSGGGGSQVQLQESGGGLV QAGGSLRLSCAASGRTFSEYAMGWFRQAPGKEREFVATISWSGG STYYTDSVKGRFTISRDNAKNTVYLQMNSLKPDDTAVYYCAAAG LGTVVSEWDYDYDYWGQGTQVTVSS |
| 244 | Q185E | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSEPKSCDKTHTCPPCPAPELLGGKKKPLDGEYFTLQIRGRERF EMFRELNEALELKDAQAGKEPGGGGGSGGGGSQVQLQESGGGLV QAGGSLRLSCAASGRTFSEYAMGWFRQAPGKEREFVATISWSGG STYYTDSVKGRFTISRDNAKNTVYLQMNSLKPDDTAVYYCAAAG LGTVVSEWDYDYDYWGQGTQVTVSSGGSGGSQVQLQESGGGLVQ AGGSLRLSCAASGRTFSEYAMGWFRQAPGKEREFVATISWSGGS TYYTDSVKGRFTISRDNAKNTVYLQMNSLKPDDTAVYYCAAAGL GTVVSEWDYDYDYWGQGTQVTVSS |
| 245 | Q209 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG |
| | | GSASTKGPSVFPLAPSSKSTSGGTAALGCLVKDYFPEPVTVSWN SGALTSGVHTFPAVLQSSGLYSLSSVVTVPSSSLGTQTYICNVN HKPSNTKVDKKVEPKSCDKTHTKKKPLDGEYFTLQIRGRERFEM FRELNEALELKDAQAGKEPGGGGGSGGGGSQVQLQESGGGLVQA GGSLRLSCAASGRTFSEYAMGWFRQAPGKEREFVATISWSGGST YYTDSVKGRFTISRDNAKNTVYLQMNSLKPDDTAVYYCAAAGLG TVVSEWDYDYDYWGQGTQVTVSSGGSGGSQVQLQESGGGLVQAG GSLRLSCAASGRTFSEYAMGWFRQAPGKEREFVATISWSGGSTY YTDSVKGRFTISRDNAKNTVYLQMNSLKPDDTAVYYCAAAGLGT VVSEWDYDYDYWGQGTQVTVSS |
| 246 | Q210 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSASTKGPSVFPLAPSSKSTSGGTAALGCLVKDYFPEPVTVSWN SGALTSGVHTFPAVLQSSGLYSLSSVVTVPSSSLGTQTYICNVN HKPSNTKVDKKVEPKSCDKTHTCPPCPAPELLGGPSVFLFPPKP KDTLMISRTPEVTCVWDVSHEDPEVKFNWYVDGVEVHNAKTKP REEQYNSTYRWSVLTVLHQDWLNGKEYKCKVSNKALPAPIEKT ISKAKGQPREPQVYTLPPSRDELTKNQVSLTCLVKGFYPSDIAV EWESNGQPENNYKTTPPVLDSDGSFFLYSKLTVDKSRWQQGNVF SCSVMHEALHNHYTQKSLSLSPGKKKKPLDGEYFTLQIRGRERF EMFRELNEALELKDAQAGKE PG |
| 247 | Q211 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCVASGSIFSINAMDWYRQAPGKQR ELVAGITSGGSTNYGDFVKGRFTISRDNAKNTVYLQMDSLKPED TAVYYCAAEVGGWGPPRPDYWGHGTQVTVSSGGGGSGGGGSAST KGPSVFPLAPSSKSTSGGTAALGCLVKDYFPEPVTVSWNSGALT SGVHTFPAVLQSSGLYSLSSVVTVPSSSLGTQTYICNVNHKPSN TKVDKKVEPKSCDKTHTCPPCPAPELLGGPSVFLFPPKPKDTLM ISRTPEVTCVWDVSHEDPEVKFNWYVDGVEVHNAKTKPREEQY |
| | | NSTYRVVSVLTVLHQDWLNGKEYKCKVSNKALPAPIEKTISKAK GQPREPQVYTLPPSRDELTKNQVSLTCLVKGFYPSDIAVEWESN GQPENNYKTTPPVLDSDGSFFLYSKLTVDKSRWQQGNVFSCSVM HEALHNHYTQKSLSLSPGKKKKPLDGEYFTLQIRGRERFEMFRE LNEALELKDAQAGKEPG |
| 248 | Q212 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCAASGRTFSEYAMGWFRQAPGKER EFVATISWSGGSTYYTDSVKGRFTISRDNAKNTVYLQMNSLKPD DTAVYYCAAAGLGTVVSEWDYDYDYWGQGTQVTVSSGGGGSGGG GSASTKGPSVFPLAPSSKSTSGGTAALGCLVKDYFPEPVTVSWN SGALTSGVHTFPAVLQSSGLYSLSSVVTVPSSSLGTQTYICNVN HKPSNTKVDKKVEPKSCDKTHTCPPCPAPELLGGKKKPLDGEYF TLQIRGRERFEMFRELNEALELKDAQAGKEPG |
| 249 | Q213 | QVQLQESGGGLVQAGGSLRLSCAASGRTFSEYAMGWFRQAPGKE REFVATISWSGGSTYYTDSVKGRFTISRDNAKNTVYLQMNSLKP DDTAVYYCAAAGLGTVVSEWDYDYDYWGQGTQVTVSSGGSGGSQ VQLQESGGGLVQAGGSLRLSCVASGSIFSINAMDWYRQAPGKQR ELVAGITSGGSTNYGDFVKGRFTISRDNAKNTVYLQMDSLKPED TAVYYCAAEVGGWGPPRPDYWGHGTQVTVSSGGGGSGGGGSAST KGPSVFPLAPSSKSTSGGTAALGCLVKDYFPEPVTVSWNSGALT SGVHTFPAVLQSSGLYSLSSVVTVPSSSLGTQTYICNVNHKPSN TKVDKKVEPKSCDKTHTCPPCPAPELLGGKKKPLDGEYFTLQIR GRERFEMFRELNEALELKDAQAGKEPG |
| 250 | REGN1098 7 Ig-TD | QVQLVESGGGVVQPGRSLRLSCAASGFTFSNYAMYWVRQAPGKG LEWVAVISYDGSNKYYADSVKGRFTISRDNSKNTLYLQMNSLRT EDTAVYYCASGSDYGDYLLVYWGQGTLVTVSS ASTKGPSVFPLA PSSKSTSGGTAALGCLVKDYFPEPVTVSWNSGALTSGVHTFPAV LQSSGLYSLSSVVTVPSSSLGTQTYICNVNHKPSNTKVDKKVEP KSCDKTHTCPPCPAPELLGGPSVFLFPPKPKDTLMISRTPEVTC VWDVSHEDPEVKFNWYVDGVEVHNAKTKPREEQYNSTYRWSV LTVLHQDWLNGKEYKCKVSNKALPAPIEKTISKAKGQPREPQVY TLPPSREEMTKNQVSLTCLVKGFYPSDIAVEWESNGQPENNYKT |
| | | TPPVLDSDGSFFLYSKLTVDKSRWQQGNVFSCSVMHEALHNHYT QKSLSLSPGKKKKPLDGEYFTLQIRGRERFEMFRELNEALELKD AQAGKEPG |
| 251 | REGN1098 7 mIg-TD | QVQLVESGGGVVQPGRSLRLSCAASGFTFSNYAMYWVRQAPGKG LEWVAVISYDGSNKYYADSVKGRFTISRDNSKNTLYLQMNSLRT EDTAVYYCASGSDYGDYLLVYWGQGTLVTVSS ASTKGPSVFPLA PSSKSTSGGTAALGCLVKDYFPEPVTVSWNSGALTSGVHTFPAV LQSSGLYSLSSVVTVPSSSLGTQTYICNVNHKPSNTKVDKKVEP KSCDKTHTAPELLGGPSVFLFPPKPKDTLMISRTPEVTCVVVDV SHEDPEVKFNWYVDGVEVHNAKTKPREEQYNSTYRWSVLTVLH QDWLNGKEYKCKVSNKALPAPIEKTISKAKGQPREPQVYTLPPS RDELTKNQVSLTCLVKGFYPSDIAVEWESNGQPENNYKTTPPVL DSDGSFFLYSKLTVDKSRWQQGNVFSCSVMHEALHNHYTQKSLS LSPGKKKKPLDGEYFTLQIRGRERFEMFRELNEALELKDAQAGK EPG |
| 252 | REGN1098 7 Fab-TD | QVQLVESGGGVVQPGRSLRLSCAASGFTFSNYAMYWVRQAPGKG LEWVAVISYDGSNKYYADSVKGRFTISRDNSKNTLYLQMNSLRT EDTAVYYCASGSDYGDYLLVYWGQGTLVTVSS ASTKGPSVFPLA PSSKSTSGGTAALGCLVKDYFPEPVTVSWNSGALTSGVHTFPAV LQSSGLYSLSSVVTVPSSSLGTQTYICNVNHKPSNTKVDKKVEP KSCDKTHTKKKPLDGEYFTLQIRGRERFEMFRELNEALELKDAQ AGKEPG |
| 253 | REGN1098 7 Lambda LC | QSALTQPASVSGSPGQSITISCTGTSSDVGGYNYVSWYQQHPGK APKLMIYDVSKRPSGVSNRFSGSKSGNTASLTISGLQSEDEADY YCNSLTSISTWVFGGGTKLTVL GQPKAAPSVTLFPPSSEELQAN KATLVCLISDFYPGAVTVAWKADSSPVKAGVETTTPSKQSNNKY AASSYLSLTPEQWKSHRSYSCQVTHEGSTVEKTVAPTECS |
| 254 | REGN1093 3 Ig-TD | QVQLVESGGGLVKPGGSLRLSCAASGFTFSDYYMSWIRQAPGKG LEWVSYITYSGSTIYYADSVKGRFTISRDNAKSSLYLQMNSLRA EDTAVYYCARDRGTTMVPFDYWGQGTLVTVSS ASTKGPSVFPLA PSSKSTSGGTAALGCLVKDYFPEPVTVSWNSGALTSGVHTFPAV LQSSGLYSLSSVVTVPSSSLGTQTYICNVNHKPSNTKVDKKVEP KSCDKTHTCPPCPAPELLGGPSVFLFPPKPKDTLMISRTPEVTC VWDVSHEDPEVKFNWYVDGVEVHNAKTKPREEQYNSTYRWSV |
| | | LTVLHQDWLNGKEYKCKVSNKALPAPIEKTISKAKGQPREPQVY TLPPSREEMTKNQVSLTCLVKGFYPSDIAVEWESNGQPENNYKT TPPVLDSDGSFFLYSKLTVDKSRWQQGNVFSCSVMHEALHNHYT QKSLSLSPGKKKKPLDGEYFTLQIRGRERFEMFRELNEALELKD AQAGKEPG |
| 255 | REGN1093 3 mIg-TD | QVQLVESGGGLVKPGGSLRLSCAASGFTFSDYYMSWIRQAPGKG LEWVSYITYSGSTIYYADSVKGRFTISRDNAKSSLYLQMNSLRA EDTAVYYCARDRGTTMVPFDYWGQGTLVTVSS ASTKGPSVFPLA PSSKSTSGGTAALGCLVKDYFPEPVTVSWNSGALTSGVHTFPAV LQSSGLYSLSSVVTVPSSSLGTQTYICNVNHKPSNTKVDKKVEP KSCDKTHTAPELLGGPSVFLFPPKPKDTLMISRTPEVTCVVVDV SHEDPEVKFNWYVDGVEVHNAKTKPREEQYNSTYRWSVLTVLH QDWLNGKEYKCKVSNKALPAPIEKTISKAKGQPREPQVYTLPPS RDELTKNQVSLTCLVKGFYPSDIAVEWESNGQPENNYKTTPPVL DSDGSFFLYSKLTVDKSRWQQGNVFSCSVMHEALHNHYTQKSLS LSPGKKKKPLDGEYFTLQIRGRERFEMFRELNEALELKDAQAGK EPG |
| 256 | REGN1093 3 Fab-TD | QVQLVESGGGLVKPGGSLRLSCAASGFTFSDYYMSWIRQAPGKG LEWVSYITYSGSTIYYADSVKGRFTISRDNAKSSLYLQMNSLRA EDTAVYYCARDRGTTMVPFDYWGQGTLVTVSS ASTKGPSVFPLA PSSKSTSGGTAALGCLVKDYFPEPVTVSWNSGALTSGVHTFPAV LQSSGLYSLSSVVTVPSSSLGTQTYICNVNHKPSNTKVDKKVEP KSCDKTHTKKKPLDGEYFTLQIRGRERFEMFRELNEALELKDAQ AGKEPG |
| 257 | REGN1093 3 Kappa LC | DIQMTQSPSSLSASVGDRVTITCQASQDITNYLNWYQQKPGKAP KLLIYAASNLETGVPSRFSGSGSGTDFTFTISGLQPEDIATYYC QQYDNLPLTFGGGTKVEIK RTVAAPSVFIFPPSDEQLKSGTASV VCLLNNFYPREAKVQWKVDNALQSGNSQESVTEQDSKDSTYSLS STLTLSKADYEKHKVYACEVTHQGLSSPVTKSFNRGEC |
| 258 | CB6 Ig-TD | EVQLVESGGGLVQPGGSLRLSCAASGFTVSSNYMSWVRQAPGKG LEWVSVIYSGGSTFYADSVKGRFTISRDNSMNTLFLQMNSLRAE DTAVYYCARVLPMYGDYLDYWGQGTLVTVSS ASTKGPSVFPLAP SSKSTSGGTAALGCLVKDYFPEPVTVSWNSGALTSGVHTFPAVL QSSGLYSLSSVVTVPSSSLGTQTYICNVNHKPSNTKVDKKVEPK |
| | | SCDKTHTCPPCPAPELLGGPSVFLFPPKPKDTLMISRTPEVTCV VVDVSHEDPEVKFNWYVDGVEVHNAKTKPREEQYNSTYRVVSVL TVLHQDWLNGKEYKCKVSNKALPAPIEKTISKAKGQPREPQVYT LPPSREEMTKNQVSLTCLVKGFYPSDIAVEWESNGQPENNYKTT PPVLDSDGSFFLYSKLTVDKSRWQQGNVFSCSVMHEALHNHYTQ KSLSLSPGKKKKPLDGEYFTLQIRGRERFEMFRELNEALELKDA QAGKEPG |
| 259 | CB6 mIg-TD | EVQLVESGGGLVQPGGSLRLSCAASGFTVSSNYMSWVRQAPGKG LEWVSVIYSGGSTFYADSVKGRFTISRDNSMNTLFLQMNSLRAE DTAVYYCARVLPMYGDYLDYWGQGTLVTVSS ASTKGPSVFPLAP SSKSTSGGTAALGCLVKDYFPEPVTVSWNSGALTSGVHTFPAVL QSSGLYSLSSVVTVPSSSLGTQTYICNVNHKPSNTKVDKKVEPK SCDKTHTAPELLGGPSVFLFPPKPKDTLMISRTPEVTCVVVDVS HEDPEVKFNWYVDGVEVHNAKTKPREEQYNSTYRWSVLTVLHQ DWLNGKEYKCKVSNKALPAPIEKTISKAKGQPREPQVYTLPPSR DELTKNQVSLTCLVKGFYPSDIAVEWESNGQPENNYKTTPPVLD SDGSFFLYSKLTVDKSRWQQGNVFSCSVMHEALHNHYTQKSLSL SPGKKKKPLDGEYFTLQIRGRERFEMFRELNEALELKDAQAGKE PG |
| 260 | CB6 Fab-TD | EVQLVESGGGLVQPGGSLRLSCAASGFTVSSNYMSWVRQAPGKG LEWVSVIYSGGSTFYADSVKGRFTISRDNSMNTLFLQMNSLRAE DTAVYYCARVLPMYGDYLDYWGQGTLVTVSS ASTKGPSVFPLAP SSKSTSGGTAALGCLVKDYFPEPVTVSWNSGALTSGVHTFPAVL QSSGLYSLSSVVTVPSSSLGTQTYICNVNHKPSNTKVDKKVEPK SCDKTHTKKKPLDGEYFTLQIRGRERFEMFRELNEALELKDAQA GKEPG |
| 261 | CB6 Kappa LC | DIVMTQSPSSLSASVGDRVTITCRASQSISRYLNWYQQKPGKAP KLLIYAASSLQSGVPSRFSGSGSGTDFTLTISSLQPEDFATYYC QQSYSTPPEYTFGQGTKLEIK RTVAAPSVFIFPPSDEQLKSGTA SVVCLLNNFYPREAKVQWKVDNALQSGNSQESVTEQDSKDSTYS LSSTLTLSKADYEKHKVYACEVTHQGLSSPVTKSFNRGEC |
| 262 | >REGN109 87 VH | QVQLVESGGGVVQPGRSLRLSCAASGFTFSNYAMYWVRQAPGKG LEWVAVISYDGSNKYYADSVKGRFTISRDNSKNTLYLQMNSLRT |
| | | EDTAVYYCASGSDYGDYLLVYWGQGTLVTVSS |
| 263 | >REGN109 87 VL | QSALTQPASVSGSPGQSITISCTGTSSDVGGYNYVSWYQQHPGK APKLMIYDVSKRPSGVSNRFSGSKSGNTASLTISGLQSEDEADY YCNSLTSISTWVFGGGTKLTVL |
| 264 | >REGN109 33 VH | QVQLVESGGGLVKPGGSLRLSCAASGFTFSDYYMSWIRQAPGKG LEWVSYITYSGSTIYYADSVKGRFTISRDNAKSSLYLQMNSLRA EDTAVYYCARDRGTTMVPFDYWGQGTLVTVSS |
| 265 | >REGN109 33 VL | DIQMTQSPSSLSASVGDRVTITCQASQDITNYLNWYQQKPGKAP KLLIYAASNLETGVPSRFSGSGSGTDFTFTISGLQPEDIATYYC QQYDNLPLTFGGGTKVEIK |
| 266 | >CB6 VH | EVQLVESGGGLVQPGGSLRLSCAASGFTVSSNYMSWVRQAPGKG LEWVSVIYSGGSTFYADSVKGRFTISRDNSMNTLFLQMNSLRAE DTAVYYCARVLPMYGDYLDYWGQGTLVTVSS |
| 267 | >CB6 VL | DIVMTQSPSSLSASVGDRVTITCRASQSISRYLNWYQQKPGKAP KLLIYAASSLQSGVPSRFSGSGSGTDFTLTISSLQPEDFATYYC QQSYSTPPEYTFGQGTKLEIK |
| 287 | Q206 | QVQLQESGGGLVQAGGSLRLSCAASGRTFSEYAMGWFRQAP GKEREFVATISWSGGSTYYTDSVKGRFTISRDNAKNTVYLQ MNSLKPDDTAVYYCAAAGLGTVVSEWDYDYDYWGQGTQVTV SSGGSGGSQVQLQESGGGLVQAGGSLRLSCVASGSIFSINA MDWYRQAPGKQRELVAGITSGGSTNYGDFVKGRFTISRDNA KNTVYLQMDSLKPEDTAVYYCAAEVGGWGPPRPDYWGHGTQ VTVSSGGGGSGGGGSKRTVAAPSVFIFPPSDEQLKSGTASV VCLLNNFYPREAKVQWKVDNALQSGNSQESVTEQDSKDSTY SLSSTLTLSKADYEKHKVYACEVTHQGLSSPVTKSFNRGEC |

### Table 33: Nucleic Acid Sequences Encoding Binding Domains (VH and VL) for Use in the Present Invention

SEQ ID NO:268 [Homo sapiens] REGN10989_VH .

CAGGTGCAGCTGGTGCAGTCTGGGGCTGAGGTGAAGAAGCCTGGGGCCTC
AGTGAAGGTCTCCTGCAAGGCTTCTGGATACATCTTCACCGGCTACTATA
TGCACTGGGTGCGACAGGCCCCTGGACAGGGGCTTGAGTGGATGGGATGG
ATCAACCCTAACAGTGGTGGCGCAAACTATGCACAGAAGTTTCAGGGCAG
GGTCACCCTGACCAGGGACACGTCCATCACCACAGTCTACATGGAACTGA
GCAGGCTGAGATTTGACGACACGGCCGTGTATTACTGTGCGAGAGGATCC
CGGTATGACTGGAACCAGAACAACTGGTTCGACCCCTGGGGCCAGGGAAC
CCTGGTCACCGTCTCCTCA

SEQ ID NO:269 [Homo sapiens] REGN10989_VL .

CAGTCTGCCCTGACTCAGCCTGCCTCCGTGTCTGGGTCTCCTGGACAGTC
GATCACCATCTCCTGCACTGGAACCAGCAGTGACGTTGGTACTTATAACT
ATGTCTCCTGGTACCAACAACACCCAGGCAAAGCCCCCAAACTCATGATT
TTTGATGTCAGTAATCGGCCCTCAGGGGTTTCTGATCGCTTCTCTGGCTC
CAAGTCTGGCAACACGGCCTCCCTGACCATCTCTGGGCTCCAGGCTGAGG
ACGAGGCTGATTATTACTGCAGCTCATTTACAACCAGCAGCACTGTGGTT
TTCGGCGGAGGGACCAAGCTGACCGTCCTA

SEQ ID NO:270 [Homo sapiens] RENG10987_VH .

CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTATGCTA
TGTACTGGGTCCGCCAGGCTCCAGGCAAGGGGCTGGAGTGGGTGGCAGTT
ATATCATATGATGGAAGTAATAAATACTATGCAGACTCCGTGAAGGGCCG
ATTCACCATCTCCAGAGACAATTCCAAGAACACGCTGTATCTGCAAATGA
ACAGCCTGAGAACTGAGGACACGGCTGTGTATTACTGTGCGAGTGGCTCC
GACTACGGTGACTACTTATTGGTTTACTGGGGCCAGGGAACCCTGGTCAC
CGTCTCCTCA

SEQ ID NO:271 [Homo sapiens] REGN10987_VL .

CAGTCTGCCCTGACTCAGCCTGCCTCCGTGTCTGGGTCTCCTGGACAGTC
GATCACCATCTCCTGCACTGGAACCAGCAGTGACGTTGGTGGTTATAACT
ATGTCTCCTGGTACCAACAACACCCAGGCAAAGCCCCCAAACTCATGATT
TATGATGTCAGTAAGCGGCCCTCAGGGGTTTCTAATCGCTTCTCTGGCTC
CAAGTCTGGCAACACGGCCTCCCTGACCATCTCTGGGCTCCAGTCTGAGG
ACGAGGCTGATTATTACTGCAACTCTTTGACAAGCATCAGCACTTGGGTG
TTCGGCGGAGGGACCAAGCTGACCGTCCTA

SEQ ID NO:272 [Homo sapiens] REGN10933_VH .

CAGGTGCAGCTGGTGGAGTCTGGGGGAGGCTTGGTCAAGCCTGGAGGGTC
CCTGAGACTCTCCTGTGCAGCCTCTGGATTCACCTTCAGTGACTACTACA
TGAGCTGGATCCGCCAGGCTCCAGGGAAGGGGCTGGAGTGGGTTTCATAC
ATTACTTATAGTGGTAGTACCATATACTACGCAGACTCTGTGAAGGGCCG
ATTCACCATCTCCAGGGACAACGCCAAGAGCTCACTGTATCTGCAAATGA
ACAGCCTGAGAGCCGAGGACACGGCCGTGTATTACTGTGCGAGAGATCGC
GGTACAACTATGGTCCCCTTTGACTACTGGGGCCAGGGAACCCTGGTCAC
CGTCTCCTCA

SEQ ID NO:273 [Homo sapiens] REGN10933_VK .

GACATCCAGATGACCCAGTCTCCATCCTCCCTGTCTGCATCTGTAGGAGA
CAGAGTCACCATCACTTGCCAGGCGAGTCAGGACATTACCAACTATTTAA
ATTGGTATCAGCAGAAACCAGGGAAAGCCCCTAAGCTCCTGATCTACGCT
GCATCCAATTTGGAAACAGGGGTCCCATCAAGGTTCAGTGGAAGTGGATC
TGGGACAGATTTTACTTTCACCATCAGCGGCCTGCAGCCTGAAGATATTG
CAACATATTACTGTCAACAGTATGATAATCTCCCTCTCACTTTCGGCGGA
GGGACCAAGGTGGAGATCAAA

SEQ ID NO:274 [Homo sapiens] REGN10934_VH .

GAGGTGCAGCTGGTGGAGTCTGGGGGAGGCTTGGTAAAGCCTGGGGGGTC
CCTTAGACTCTCCTGTGCAGCCTCTGGAATCACTTTCAGTAACGCCTGGA
TGAGTTGGGTCCGCCAGGCTCCAGGGAAGGGGCTGGAGTGGGTTGGCCGT
ATTAAAAGCAAAACTGATGGTGGGACAACAGACTACGCCGCACCCGTGAA
AGGCAGATTCACCATCTCAAGAGATGATTCAAAAAACACGCTGTATCTAC
AAATGAACAGCCTGAAAACCGAGGACACAGCCGTGTATTACTGTACCACA
GCGAGGTGGGACTGGTACTTCGATCTCTGGGGCCGTGGCACCCTGGTCAC
TGTCTCCTCA

SEQ ID NO:275 [Homo sapiens] REGN10934_VK .

GACATCCAGATGACCCAGTCTCCATCCTCCCTGTCTGCATCTGTAGGAGA
CAGAGTCACCATCACTTGCCAGGCGAGTCAGGACATTTGGAATTATATAA
ATTGGTATCAGCAGAAACCAGGGAAGGCCCCTAAGCTCCTGATCTACGAT
GCATCCAATTTGAAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGCATGATGATCTCCCTCCGACCTTCGGCCAA
GGGACCAAGGTGGAAATCAAA

SEQ ID NO:276 [Homo sapiens] REGN10977_VH .

CAGGTGCAGCTGGTGCAGTCTGGGGCTGAGGTGAAGAAGCCTGGGTCCTC
GGTGAAGGTCTCCTGCAAGGCTTCTGGAGGCACCTTCAGCAGCTATGCTA
TCACCTGGGTGCGACAGGCCCCTGGACAAGGGCTTGAGTGGATGGGAAGG
ATCATCCCTATGTTTGGTATAGCAAACTACGCACAGAAGTTCCAGGGCAG
AGTCACGATTACCGCGGACAAATCCACGAGCACAGCCTACATGGAGGTGA
GCAGCCTGAGATCTGAGGACACGGCCGTGTATTACTGTGCGAGAACACCG
TTTTACTATGATAGTAGTGGTTATTACCTTGACTACTGGGGCCAGGGAAC
CCTGGTCACCGTCTCCTCA

SEQ ID NO:277 [Homo sapiens] REGN10977_VK .

GAAATTGTGTTGACGCAGTCTCCAGGCACCCTGTCTTTGTCTCCAGGGGA
AAGAGCCACCCTCTCTTGCAGGGCCAGTCAGAGTGTTAGCAGCAACTACT
TAGCCTGGTACCAGCAGAGTCCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTTTATTACTGTCAGCAGTATGTTAGGTCACCCCGGACGTTCGGC
CAAGGGACCAAGGTGGAAATCAAA

SEQ ID NO:278 [Homo sapiens] REGN10964_VH .

CAGGTGCAGCTGCAGGAGTCGGGCCCAGGACTGGTGAAGCCTTCGGAGAC
CCTGTCCCTCACCTGCACTGTCTCTGGTGGCTCCATCAATAATTACTACT
GGATCTGGATCCGGCAGCCCCCAGGGAAGGGACTGGAGTGGATTGGGTAT
ATCTATCACAGTGGGAGCACCAACTACAACCCCTCCCTCAAGAGTCGAGT
CACCATATCAGTAGACACGTCCAAGAACCAATTCTCCCTGAAGGTGAGCT
CTGTGACCGCTGCGGACACGGCCGTGTATTACTGTGCGAATATGGTTCGG
GGAGTTTATGAAGATGACTACTGGGGCCAGGGAACCCTGGTCACCGTCTC
CTCA

SEQ ID NO:279 [Homo sapiens] REGN10964_VK .

GACATCCAGATGACCCAGTCTCCATCCTCCCTGTCTGCATCTGTAGGAGA
CAGAGTCACCATCACTTGCCGGGCAAGTCAGAGCATTAGCAACTATTTAA
ATTGGTATCAGCAGAAACCAGGGAAAGCCCCTAAGCTCCTGATCTTTGCT
GCCTCCAGTTTGCGAAGTGGGGTCCCATCAAGGTTCAGTGGCAGTGGATC
TGGGACAGATTTCACTCTCACCATCAGCAGTCTGCACCCTGAAGATTTTG
CAACTTACTACTGTCAACAGAATTACAATACCCCCCTCACTTTCGGCCCT
GGGACCAAAGTGGATATCAAA

SEQ ID NO:280 [Homo sapiens] REGN10954_VH .

GAGGTGCAGCTGGTGGAGTCTGGGGGAGGCTTGGTCCAGCCTGGGGGGTC
ACTGAGACTCTCCTGTGCAGCCTCTGGAATCACCGTCAGTAGCAACTACA
TGAGCTGGGTCCGCCAGGCTCCAGGGAAGGGGCTGGAGTGGGTCTCACTT
ATTTATAGTGGTGGTAGCACATACTACGCAGACTCCGTGAAGGGCAGATT
CACCATCTCCAGAGACAATTCCAAGAACACGCTGTATCTTCAAATGAACA
GCCTGAGAGTCGAGGACACGGCTGTGTATTACTGTGCGAGAGATCACGGT
ATGGCAGCAGCGGGGTATAATTACTGGGGCCAGGGAACCCTGGTCACCGT
CTCCTCA

SEQ ID NO:281 [Homo sapiens] REGN10954_VK .

GACATCCAGATGACCCAGTCTCCATCCTCCCTGTCTGCGTCTGTAGGAGA
CAGAGTCACCATCACTTGCCAGGCGAGTCAGGACATTAACAAATATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAACAGCCTGCAGCCTGAAGATTTTG
CAACATATTTCTGTCAACAGTATGATAATCTCCCTCCAGCGTTCGGCCAA
GGGACCAAGGTGGAAATCAAA

SEQ ID NO:282 [Homo sapiens] REGN10984_VH .

GAGGTGCAGCTGGTGGAGTCTGGGGGAGGCTTGGTCCAGCCTGGGGGGTC
CCTGAGACTCTCCTGTGCAGCCTCTGGACTCACCGTCAATCGCAACTACA
TGAGCTGGGTCCGCCAGGCTCCAGGGAAGGGGCTGGAGTGGGTCTCACTT
ATTTATAGCGGTGGTAGCACATACTACGCAGACTCCGTTAAGGGCCGATT
CACCATCTCCAGAGACAATTCCAAGAACACGCTGTATCTTCAAATGAACA
GCCTGAGACTTGAGGACACGGCTGTGTATTACTGTGCGAGAGGTGAACTG
GGGATCCCCTACGGTATGGACGTCTGGGGCCAAGGGACCACGGTCACCGT
CTCCTCA

SEQ ID NO:283 [Homo sapiens] REGN10984_VL .

CAGTCTGTGTTGACGCAGCCGCCCTCAGTGTCTGCGGCCCCAGGACAGAA
GGTCACCATCTCCTGCTCTGGAAGCAGCTCCAACATTGGGAATAATTATG
TATCCTGGTACCAGCAGCTCCCAGGAACAGCCCCCAAACTCCTCATTTAT
GACAATAATAAGCGACCCTCAGGGATTCCTGACCGCTTCTCTGGCTCCAA
GTCTGGCACGTCAGCCACCCTGGGCATCACCGGACTCCAGACTGGGGACG
AGGCCGATTATTACTGCGGAACATGGGATAGCAGCCTGAGTGCTGGGGTG
TTCGGCGGAGGGACCAAGCTGACCGTCCTA

SEQ ID NO:284 [Homo sapiens] REGN10986_VH .

GAGGTGCAGCTGGTGGAGTCTGGGGGAGGCTTGGTCCAGCCTGGGGGGTC
CCTGAGACTCTCCTGTGCAGCCTCTGAATTCACCGTCAGTAGCAACTACA
TGAGCTGGGTCCGCCAGGCTCCAGGGAAGGGGCTAGAGTGGGTCTCAGTT
ATTTATAGCGGTGGTAGCACATTTTACGCAGACTCCGTGAAGGGCAGATT
CACCATCTCCAGAGACAATTCCAAGAACACGTTGTATCTTCAAATGAACA
GTCTGAGAGCCGAGGACACGGCTGTATATTACTGTGCGAGAGCTCTTCCC
TACGGTGACTTGCATTTTGACTACTGGGGCCAGGGAACCCTGGTCACCGT
CTCCTCA

SEQ ID NO:285 [Homo sapiens] REGN10986_VL .

CAGTCTGTGCTGACGCAGCCGCCCTCAGTGTCTGGGGCCCCAGGGCAGAG
GGTCACCATCTCCTGCACTGGGAGCAGCTCCAACATCGGGACAGGTTATG
ATGTACACTGGTACCAGCAGCTTCCAGGAACAGCCCCCAAACTCCTCATC
TATGGTAACACCAATCGGCCCTCAGGGGTCCCTGACCGATTCTCTGGCTC
CAAGTCTGGCACCTCAGCCTCCCTGGCCATCACTGGGCTCCAGGCTGAGG
ATGAGGCTGATTATTACTGCCAGTCCTATGACAGCAGCCTGAGTGACTCT
TATGTCTTCGGAACTGGGACCAAGGTCACCGTCCTA

TABLE 34

Amino acid sequences of a binding domain (Nb-112, SEQ ID NO: 288) and polypeptides (SEQ ID NOs: 289 & 290) for use in the present invention

| SEQ ID No: | Quad Name | AMINO ACID SEQUENCE |
|---|---|---|
| 288 | Q231 | DVQLQESGGGLVQPGGSLRLSCAASGLTLDYYAIGWFRQA PGKEREGVSCISSSDGSTYYADSVKGRFTTSRDNAKNTVY LQMNSLKPEDTAVYYCAAVPSTYYSGTYYYTCHPGGMDYW GKGTQVTVSS |
| 289 | Q232 | DVQLQESGGGLVQPGGSLRLSCAASGLTLDYYAIGWFRQA PGKEREGVSCISSSDGSTYYADSVKGRFTTSRDNAKNTVY LQMNSLKPEDTAVYYCAAVPSTYYSGTYYYTCHPGGMDYW GKGTQVTVSSGGGGSGGGGSKKKPLDGEYFTLQIRGRERF EMFRELNEALELKDAQAGKEPG |
| 290 | Q233 | DVQLQESGGGLVQPGGSLRLSCAASGLTLDYYAIGWFRQA PGKEREGVSCISSSDGSTYYADSVKGRFTTSRDNAKNTVY LQMNSLKPEDTAVYYCAAVPSTYYSGTYYYTCHPGGMDYW GKGTQVTVSSGGSGGSDVQLQESGGGLVQPGGSLRLSCAA SGLTLDYYAIGWFRQAPGKEREGVSCISSSDGSTYYADSV KGRFTTSRDNAKNTVYLQMNSLKPEDTAVYYCAAVPSTYY SGTYYYTCHPGGMDYWGKGTQVTVSSGGGGSGGGGSKKKP LDGEYFTLQIRGRERFEMFRELNEALELKDAQAGKEPG |

TABLE 35

Amino acid sequences of Nb-112 based Quad polypeptides with an intact hinge region

| SEQ ID No: | Quad Name | AMINO ACID SEQUENCE |
|---|---|---|
| 291 | Q246 | DVQLQESGGGLVQPGGSLRLSCAASGLTLDYYAIGWFRQA PGKEREGVSCISSSDGSTYYADSVKGRFTTSRDNAKNTVY LQMNSLKPEDTAVYYCAAVPSTYYSGTYYYTCHPGGMDYW GKGTQVTVSSGGGGSGGGGSEPKSCDKTHTCPPCPAPELL GGKKKPLDGEYFTLQIRGRERFEMFRELNEALELKDAQAG KEPG |
| 292 | Q247 | DVQLQESGGGLVQPGGSLRLSCAASGLTLDYYAIGWFRQA PGKEREGVSCISSSDGSTYYADSVKGRFTTSRDNAKNTVY LQMNSLKPEDTAVYYCAAVPSTYYSGTYYYTCHPGGMDYW GKGTQVTVSSGGSGGSDVQLQESGGGLVQPGGSLRLSCAA SGLTLDYYAIGWFRQAPGKEREGVSCISSSDGSTYYADSV KGRFTTSRDNAKNTVYLQMNSLKPEDTAVYYCAAVPSTYY SGTYYYTCHPGGMDYWGKGTQVTVSSGGGGSGGGGSEPKS CDKTHTCPPCPAPELLGGKKKPLDGEYFTLQIRGRERFEM FRELNEALELKDAQAGKEPG |

### Table 36: Antibody Single Variable Domains

(a) The amino acid sequence of an anti-hRSV protein F nanobody for use as a binding domain in the present invention); there is provided a polypeptide or multimer as described herein comprising at least 4 copies of the nanobody (eg, for administration to a human or animal subject for treating or preventing RSV infection or a symptom thereof in the subject; also provided is a composition as described herein which comprises the multimer). SEQ ID NO: 293

EVQLVESGGGLVQAGGSLSISCAASGGSLSNYVLGWFRQAPGKEREFVAA
INWRGDITIGPPNVEGRFTISRDNAKNTGYLQMNSLAPDDTAVYYCGAGT
PLNPGAYIYDWSYDYWGRGTQVTVSS

(b) An antibody single variable domain (binding domain) herein may comprise a HCDR3 that comprises SEQ ID NO: 294 (which is the HCDR3 sequence of Nb1 1-59); there is provided a polypeptide or multimer as described herein comprising at least 4 copies of the domain (eg, for administration to a human or animal subject for treating or preventing SARS-CoV-2 infection or a symptom thereof in the subject; also provided is a composition as described herein which comprises the multimer). SEQ ID NO: 294

APSQTYGGSWYWDPIGD

TABLE 37

Exemplary Inhalable Compositions

| | | | |
|---|---|---|---|
| | Composition 1 (1 mg/ml of Quad) | Composition 2 (0.25 mg/ml of Quad) | Composition 3 (0.5 mg/ml of Quad) |
| Particle Size (µm) | 2.90 - 3.23 | 3.07 - 3.42 | 3.45 - 3.79 |
| Respirable Component | 67.66 - 73.50 | 71.78 - 76.69 | 62.32 - 66.90 |
| (0.5 - 5.0 µm) | | | |
| (% of composition) | | | |
| Coarse particles | 27.00 - 31.11 | 23.62 - 28.21 | 32.31 - 36.12 |
| (>4.7 µm) | | | |
| % of composition) | | | |
| Fine particles | 66.33 - 72.07 | 68.58 - 73.84 | 59.36 - 64.17 |
| (<4.7 µm) | | | |
| % of composition) | | | |
| Ultra-fiine Particles | 5.91 - 9.93 | 1.85 - 4.19 | 2.36 - 4.51 |
| (<1.0 m) | | | |
| (% of cocomposition) | | | |

1. A protein multimer comprising 4 copies of a binding site, wherein the binding site is capable of binding to a virus spike protein of a coronavirus.

2. The multimer of claim 1, wherein said virus is a first virus and the multimer is capable of binding to a second coronavirus, wherein the first and second viruses are different virus strains; optionally wherein the viruses are different SARS-CoV-2 strains.

3. The multimer of claim 2, wherein the viruses have different forms of virus spike proteins and the multimer is capable of binding to the spike proteins.

4. The multimer of any preceding claim, wherein the binding site is an antibody single variable domain.

5. The multimer of any preceding claim,wherein the binding site is
a) an antigen binding site that is capable of binding to a SARS-CoV-2 spike glycoprotein; or
b) an ACE2 or TMPRSS2 peptide.

6. The multimer of any preceding claim, wherein the binding site is capable of binding to the inner face of the RBD (receptor-binding domain) of SARS-CoV-2 spike.

7. The multimer of any preceding claim
a) wherein the binding site comprises (i) an antibody VH domain comprising the SEQ ID NO: 307 or (ii) an antibody VH domain that comprises an amino acid sequence that is at least 70% identical to SEQ ID NO: 307 or that competes in an in vitro competition assay with the antibody VH domain of (i) for binding to SARS-CoV-2 spike;
b) comprising 4 copies of an antigen binding site of an antibody, wherein the antibody is selected from a VH domain comprising the amino acid sequence of SEQ ID NO: 288 (or an antibody variable domain comprising an amino acid sequence that is at least 80% identical to SEQ ID NO: 288), REGN10987, REGN10933 and CB6; or
c) comprising 4 copies of an antigen binding site of an antibody, wherein the multimer comprises a dimer of the antibody, wherein the antibody is selected from REGN10987, REGN10933 and CB6.

8. The multimer of any preceding claim, wherein the multimer comprises at least 4 copies of a polypeptide, wherein the polypeptide comprises a self-assembly multimerization domain (SAM) (optionally a tetramerization domain, TD) and one or more copies of the antigen binding site.

9. The multimer of any one of claims 1-7, wherein the multimer is a protein dimer comprising 2 copies of
a) the polypeptide defined in claim 8, optionally wherein the polypeptide comprises (in N- to C-terminal direction) BD-BD-SAM, wherein BD is the binding site; or
b) a polypeptide comprising one or more copies of the binding site and an antibody Fc region, wherein the multimer is a dimer and the Fc regions of the 2 polypeptide copies are associated together in the dimer.

10. The multimer of claim 9, wherein the multimer comprises
a) a first polypeptide comprising, in N- to C-terminal direction, BD-hinge-TD; BD-optional linker-BD-Hinge-TD; or BD-optional linker-CH1-Hinge-TD; or
b) a TD and one or more copies of BD, the polypeptide comprising, in N- to C-terminal direction
(i) BD-TD;
(ii) TD-BD;
(iii) BD-BD-TD;
(iv) TD-BD-BD;
(v) BD-TD-BD-BD;
(vi) BD-BD-TD-BD; or
(vii) BD-BD-TD-BD-BD.

11. The multimer of any preceding claim, wherein the binding of the multimer (first multimer) to the first virus spike protein is stronger than the binding of a second multimer to the first virus spike protein, wherein the second multimer comprises 2 (but no more than 2) copies of said binding site and wherein
a) the first multimer binds to the first virus spike protein with an OD₄₅₀ from 1 to 3 in an ELISA assay in which the spike protein is at a concentration of 1 nM in the assay (and optionally the second multimer binds to the first virus spike protein with an OD₄₅₀ less than 0.5 in an ELISA assay in which the spike protein is at a concentration of 1 nM in the assay);
b) the first multimer (i) binds to a first virus spike protein trimer with an OD₄₅₀ from 2 to 3 in an ELISA assay in which the spike protein is at a concentration of 1 nM in the assay and (ii) binds to a first virus spike protein monomer with an OD₄₅₀ from 1 to 2 in an ELISA assay in which the spike protein is at a concentration of 1 nM in the assay; and/or
c) the first multimer binds to the first virus spike protein with an OD₄₅₀ from 2 to 3 in an ELISA assay in which the spike protein is at a concentration of 10 nM in the assay (and optionally the second multimer binds to the first virus spike protein with an OD₄₅₀ from 1 to 2 in an ELISA assay in which the spike protein is at a concentration of 10 nM in the assay).

12. The multimer of any preceding claim, wherein the multimer comprises 6 copies of the binding site.

13. An inhalable pharmaceutical composition comprising a multimer of any preceding claim, optionally wherein the composition is a nebulised composition.

14. The composition of claim 13, wherein
(i) the composition comprises particles of the multimer and
(a) at least 20% of multimer particles are in the size range >4.7 µm, and optionally no more than 5.0 µm; and
(b) at least 50% of multimer particles are in the size range <4.7 µm, and optionally at least 15% of multimer particles are in the size range <1.0 um; or
(ii) the composition comprises particles of the multimer and the median mass aerodynamic particle diameter (MMAD) is 3 to 3.5 µm.

15. The composition of claim 13 or 14 further comprising an anti-inflammatory medicament.

16. An inhalation device (optionally a nebuliser or inhaler) comprising the composition of any of claims 13 to 15.

17. A mixture of at least 2 different multimers as recited in any one of claims 1-12, wherein a first of said multimers comprises 4 copies of an antigen binding site that is capable of binding to a first spike antigen; and a second of said multimers comprises 4 copies of an antigen binding site that is capable of binding to a second spike antigen, and the antigens are different.

18. A method of expanding the antigen binding specificity of a binding site, wherein the binding site binds or neutralises a first antigen, but not a second antigen when the binding site is comprised in monovalent form by a protein that specifically binds to the first antigen, the method comprising providing a plurality of copies of a polypeptide, and multimerising at least 4 of the polypeptides to produce a multimer comprising at least 4 copies of the polypeptide, wherein the polypeptide comprises one, two or more copies of the binding site, whereby binding sites of the multimer are capable of binding the first and second antigens; optionally wherein the multimer is according to any one of claims 1-12.

19. A multimer obtainable by the method of any one of claims 18 wherein the multimer is for targeting a virus whose antigens evolve through mutation during viral infection of a human subject, optionally for treating a coronavirus infection.

20. A pharmaceutical composition comprising a multimer of any one of claims 1-12 and 19, optionally wherein the composition is comprised by a sterile medical container or device, such as a syringe, vial, inhaler or injection device.

21. The multimer or composition of any one of claims 1-12, 19 and 20 for use as a medicament, optionally for treating or preventing viral pneumonia in a human or animal subject, such as wherein the subject is suffering from or is at risk of suffering from a coronavirus infection.

22. A method of binding multiple copies of an antigen, the method comprising combining the copies with a multimer or composition of claims 1-12, 19 and 21, wherein the copies are bound by the multimer, and optionally the method comprising isolating the multimer bound to the antigen copies.

23. A method of detecting the presence of anti-first antigen antibodies in a bodily fluid sample of a human or animal, the method comprising carrying out an ELISA assay, wherein the assay comprises
a) Optionally diluting the serum sample from 10 to 10⁶-fold;
b) contacting the first antigen with the sample (optionally which has been diluted in step (a)) whereby anti-first antigen antibodies present in the sample bind to the first antigen (eg, spike protein) to produce antigen/antibody complexes; and
c) contacting and binding the first antigen or anti-first antigen antibodies with copies of the multimer defined in one of claims 1-12, 19 and 21; and
d) detecting multimer bound to antigen/antibody complexes, such as by determining optical density;
wherein the steps can be carried out in the order (a) (b) (c) and (d) or (a) (c) (b) and (d), or wherein steps (b) and (c) are carried out simultaneously and between steps (a) and (d).

24. A method for detecting the presence of an antigen in a sample, the method comprising combining the sample with a multimer as defined in one of claims 1-12, 19 and 21, allowing antigen in the sample to bind multimers to form antigen/multimer complexes and detecting antigen/multimer complexes.

25. A method of expanding a utility of an antigen binding site, the method comprising producing a multimer as defined in one of claims 1-12, 19 and 21, wherein the multimer comprises at least 4 copies of the binding site.

26. A method for the treatment or prevention of a disease or condition in a human or animal subject), the method comprising administering to the subject a plurality of multimers as defined in one of claims 1-12, 19 and 21.

27. A multimer according to any one of claims 1-12, 19 and 21 that is capable of binding to different forms of a virus spike protein for treating, preventing or reducing in a human or animal infection by a virus comprising a first form of spike protein, and for treating, preventing or reducing infection by a virus comprising a second form of the spike protein.

28. A multimer according to any one of claims 1-12, 19 and 21 that is capable of binding to different forms of a virus spike protein for treating or preventing or reducing a seasonal viral infection in a human or animal.

29. A medicament for administration to a human or animal subject for treating or preventing a seasonal virus, wherein the medicament comprises a plurality of multimers according to any one of claims 1-12, 19 and 21, wherein the medicament comprises a pharmaceutically acceptable diluent, carrier or excipient.

30. The medicament of claim 29, wherein the medicament is a multi-seasonal anti-viral medicament comprising a plurality of said multimers, wherein the multimers are capable of binding to first and second strains of the virus, wherein the strains differ in a surface-exposed antigen (optionally spike) to which the multimers can bind.

31. A multimer according to any one of claims 1-12, 19 and 21 for use in a method for reducing the first virus in an animal to reduce a zoonotic population of viruses that are transmissible to humans, wherein the viruses are capable of causing a disease or condition (or death) in humans.

32. The multimer or medicament of any one of claims 27-31, wherein each virus is a SARS virus, optionally SARS-Cov, SARS-Cov-2 or MERS-Cov.
